SEQUENCE LISTING (110 sequences)

SEQ ID NO: 1 - DNA, 447 bp, Homo sapiens
atgtcgagtccgcagaggaggaaggctatgccctgggcactgtcactgcttctcatgggcttccagctcctggtgacttatgcctggtgttctgaagaggaaatgggtggtaataataaaatagtccaggatcctatgttcctcgccacagtggagtttgccttgaacactttcaacgtgcagagcaaggaggagcatgcctacaggctgttgcgcgtcctgagttcatggagggaggatagcatggacagaaagatggtgttctccatgaatctgcaactgcgccaaaccgtatgtaggaaatttgaagatgacattgacaactgcccttttcaagaaagcctggagctgaacaacaccttcagctgcttcttcactgttgaaaccatgccctggaagacatattttgaactcctgaacaagacctgctcagaggggctctcctga

SEQ ID NO: 2 - DNA, 480 bp, Homo sapiens
atgtcgagtccgcagaggaggaaggctatgccctgggcactgtcactgcttctcatgggcttccagctcctggtgacttatgcctggtgttctgaagaggaaatgggtggtaataataaaatagtccaggatcctatgttcctcgccacagtggagtttgccttgaacactttcaacgtgcagagcaaggaggagcatgcctacaggctgttgcgcgtcctgagttcatggagggaggatagcatggacagaaagtggcgaggtaagatggtgttctccatgaatctgcaactgcgccaaaccgtatgtaggaaatttgaagatgacattgacaactgcccttttcaagaaagcctggagctgaacaacgtaagacagggcatcagctttcctcaggtccacagctgtggatgctgcatggggtgtggtgtgggcacaggagcagctgacaaagccattccgagggacaaagggaagtga

SEQ ID NO: 3 - DNA, 2349 bp, Homo sapiens
atggcccgggccagggccggggcgctgctggcgctttgggtgctcggggccgccgcgcatccgcagtgcctggacttcaggccgcccttccggccgacgcagccgctgcgcctctgcgcgcagtactcggacttcggctgctgcgatgaggggcgcgacgccgagctgacccgccgcttctgggccctggcgagccgcgtggacgccgccgagtgggccgcgtgcgccggctacgcgagggacctgctgtgccaggaatgctcgccgtatgcagcccacctctatgacgccgaggacccattcacgcccctgcgcacggtgcccgggctctgccaggattactgcctggacatgtggcataagtgccgggggctgttccgtcacctgtcaactgaccaggagctctgggcgctggagggcaaccttgccaggttctgccgctacctgtccctggatgacacggactactgcttcccttacctgctggtcaacaagaacctcaactcaaacctgggccacgtggtagccgatgccaagggctgcctgcagctgtgcctggaggaggtggccaacgggctgcgcaaccccgtggccatggtccatgccagggatggcacccaccgcttcttcgtggccgagcaggtggggctggtgtgggcctacctgcccgaccgctcgaggctggggaagcctttcctgaacatcagccgggtggtgctcacctcgccctgggagggtgacgagcgtggcttcctgggcattgccttccaccccagcttccagcacaaccgcaggctctacgtctactactcagtgggtatccgcagcagtgagtggatccgcatcagcgagttcagagtctccgaggatgacgagaacgccgtggaccacagctctgagaggataatcctggaggtcaaagaaccagcctcaaaccacaacgggggccagctgcttttcggggatgacgggtacctctacatcttcactggagatggcgggatggccggagacccctttgggacatttggaaatgcccaaaacaagtcggcgctgctgggcaaggtgctgcgcatcgacgtggaccgtaaggagcgcggcctgccctacggcatcccgcccgacaacccgttcgtgggcgaccccgcggcgcagcccgaggtctacgccctaggcgtgcgcaacatgtggcgctgctccttcgaccgtggcgacccctcctcgggcactggccgcgggcgcctcttctgcggcgacgtgggccagaacaagttcgaggaggtggacgtggtggagcgcggcggcaactatggctggcgcgcgcgcgaagggttcgagtgctacgaccgcagcctgtgcgccaacacctctctcaatgacttgctgccgattttcgcctacccgcacacggttggcaagtcggtcacagggggctacgtgtaccggggctgcgagtaccccaacctgaacggcctctacatttttggggatttcatgagcgggcgtctgatgtccctccaagagaacccagggacaggccagtggcagtacagtgagatctgcatgggccacggccagacctgtgagttcccaggcctcatcaacaactactacccgtacatcatctccttcggggaggacgaggccggggagctgtacttcatgtcgacaggggagccgagtgccacagctccacgcggagttgtctacaaaataattgacgcatccaggcgggcaccacctggcaaatgtcagatccagcctgctcaggtgaagatcagaagccgtctcatcccctttgtgcccaaagaaaagttcatcccgaagacacggagcaccccgcggcctacagcgcgggcgcccacgcgggcgccccgccgagggcgccccacggccgctccccccgcgccaaccccgcggccagcgcggcccacccagcagccagggagccggaggggcggcgggcggcggcgggggcggctgaactcggcgagccgggcgttccgggatggcgaggtgcgcctggtgcggcccgcgggcctgagctctggcagcgggcgcgtggaggtgttcgtgggcggacgctggggcaccgtgtgcgacgactcctggaacatcagcggcgccgccgtcgtgtgtcgccagctggggtttgcctacgccgtgcgcgccgtcaagagagccgagttcggccagggcggctcgctgcccattctgctggacgatgtgcgctgcgcgggctgggagcggaacctgctggagtgccagcacaacggcgtgggcacccacaactgcgagcacgacgaggatgcgggcgtcgtgtgcagccaccagaaccccgacctgtag

SEQ ID NO: 4 - DNA, 2301 bp, Homo sapiens
atggcccgggccagggccggggcgctgctggcgctttgggtgctcggggccgccgcgcatccgcagtgcctggacttcaggccgcccttccggccgacgcagccgctgcgcctctgcgcgcagtactcggacttcggctgctgcgatgaggggcgcgacgccgagctgacccgccgcttctgggccctggcgagccgcgtggacgccgccgagtgggccgcgtgcgccggctacgcgagggacctgctgtgccagtccgtggagtggacagacatgcaaagagataatgaagtcctagccaagctgactggctggagcgcccctggcgacggagcagtgactgctgtagagaactcaccctctctggactaccctggtctgggcaccacatttacgtcgtgtgaatgctcgccgtatgcagcccacctctatgacgccgaggacccattcacgcccctgcgcacggtgcccgggctctgccaggattactgcctggacatgtggcataagtgccgggggctgttccgtcacctgtcaactgaccaggagctctgggcgctggagggcaaccttgccaggttctgccgctacctgtccctggatgacacggactactgcttcccttacctgctggtcaacaagaacctcaactcaaacctgggccacgtggtagccgatgccaagggctgcctgcagctgtgcctggaggaggtggccaacgggctgcgcaaccccgtggccatggtccatgccagggatggcacccaccgcttcttcgtggccgagcaggtggggctggtgtgggcctacctgcccgaccgctcgaggctggggaagcctttcctgaacatcagccgggtggtgctcacctcgccctgggagggtgacgagcgtggcttcctgggcattgccttccaccccagcttccagcacaaccgcaggctctacgtctactactcagtgggtatccgcagcagtgagtggatccgcatcagcgagttcagagtctccgaggatgacgagaacgccgtggaccacagctctgagaggataatcctggaggtcaaagaaccagcctcaaaccacaacgggggccagctgcttttcggggatgacgggtacctctacatcttcactggagatggcgggatggccggagacccctttgggacatttggaaatgcccaaaacaagtatgttcagcttttgattggcttgtgggttggtctccatatccctgggcttctcatactcttccagaggtcggcgctgctgggcaaggtgctgcgcatcgacgtggaccgtaagggagcgcggcctgccctacggcatcccgcccgacaacccgttcgtgggcgaccccgcggcgcagccgaggtctacgccctaggcgtgcgcaacatgtggcgctgctccttcgaccgtggcgacccctcctcgggcactggccgcgggcgcctcttctgcggcgacgtgggccagaacaagttcgaggaggtggacgtggtggagcgcggcggcaactatggctggcgcgcgcgcgaagggttcgagtgctacgaccgcagcctgtgcgccaacacctctctcaatgacttgctgccgattttcgcctacccgcacacggttggcaagtcggtcacagggggctacgtgtaccggggctgcgagtaccccaacctgaacggcctctacatttttggggatttcatgagcgggcgtctgatgtccctccaagagaacccagggacaggccagtggcagtacagtgagatctgcatgggccacggccagacctgtgagttcccaggcctcatcaacaactactacccgtacatcatctccttcggggaggacgaggccggggagctgtacttcatgtcgacaggggagccgagtgccacagctccacgcggagttgtctacaaaataattgacgcatccagccgggcgttccgggatggcgaggtgcgcctggtgcggcccgcgggcctgagctctggcagcgggcgcgtggaggtgttcgtgggcggacgctggggcaccgtgtgcgacgactcctggaacatcagcggcgccgccgtcgtgtgtcgccagctggggtttgcctacgccgtgcgcgccgtcaagagagccgagttcggccagggcggctcgctgcccattctgctggacgatgtgcgctgcgcgggctgggagcggaacctgctggagtgccagcacaacggcgtgggcacccacaactgcgagcacgacgaggatgcgggcgtcgtgtgcagccaccagaaccccgacctgtag

SEQ ID NO: 5 - DNA, 1821 bp, Homo sapiens
atgcttcacacggccatatcatgctggcagccattcctgggtctggctgtggtgttaatcttcatgggatccaccattggctgccccgctcgctgtgagtgctctgcccagaacaaatctgttagctgtcacagaaggcgattgatcgccatcccagagggcattcccatcgaaaccaaaatcttggacctcagtaaaaacaggctaaaaagcgtcaaccctgaagaattcatatcatatcctctgctggaagagatagacttgagtgacaacatcattgccaatgtggaaccaggagcattcaacaatctctttaacctgcgttccctccgcctaaaaggcaatcgtctaaagctggtccctttgggagtattcacggggctgtccaatctcactaagcttgacattagtgagaataagattgtcattttactagactacatgttccaagatctacataacctgaagtctctagaagtgggggacaatgatttggtttatatatcacacagggcattcagtgggcttcttagcttggagcagctcaccctggagaaatgcaacttaacagcagtaccaacagaagccctctcccacctccgcagcctcatcagcctgcatctgaagcatctcaatatcaacaatatgcctgtgtatgcctttaaaagattgttccacctgaaacacctagagattgactattggcctttactggatatgatgcctgccaatagcctctacggtctcaacctcacatccctttcagtcaccaacaccaatctgtctactgtacccttccttgcctttaaacacctggtatacctgactcaccttaacctctcctacaatcccatcagcactattgaagcaggcatgttctctgacctgatccgccttcaggagcttcatatagtgggggcccagcttcgcaccattgagcctcactccttccaagggctccgcttcctacgcgtgctcaatgtgtctcagaacctgctggaaactttggaagagaatgtcttctcctcccctagggctctggaggtcttgagcattaacaacaaccctctggcctgtgactgccgccttctctggatcttgcagcgacagcccaccctgcagtttggtggccagcaacctatgtgtgctggcccagacaccatccgtgagaggtctttcaaggatttccatagcactgccctttctttttactttacctgcaaaaaacccaaaatccgtgaaaagaagttgcagcatctgctagtagatgaagggcagacagtccagctagaatgcagtgcagatggagacccgcagcctgtgatttcctgggtgacaccccgaaggcgtttcatcaccaccaagtccaatggaagagccaccgtgttgggtgatggcaccttggaaatccgctttgcccaggatcaagacagcgggatgtatgtttgcatcgctagcaatgctgctgggaatgataccttcacagcctccttaactgtgaaaggattcgcttcagatcgttttctttatgcgaacaggacccctatgtacatgaccgactccaatgacaccatttccaatggcaccaatgccaatactttttccctggaccttaaaacaatactggtgtctacagctatgggctgcttcacattcctgggagtggttttattttgttttcttctcctttttgtgtggagccgagggaaaggcaagcacaaaaacagcattgaccttgagtatgtgcccagaaaaaacaatggtgctgttgtggaaggggaggtagctggacccaggaggttcaacatgaaaatgatttga

SEQ ID NO: 6 - DNA, 1821 bp, Homo sapiens; identical to SEQ ID NO: 5

SEQ ID NO: 7 - DNA, 324 bp, Homo sapiens
atgcccagtccaatggaacacaccatggaaaccgtgttgtttacgtttcacagatttgccggggataaaggctacttaatgaaggagggcctgaaagtactcatgggaaaggagttccttggatttttggagaatcaaaaagaccctctggctgcagacataacaatgaaggacatggaccagtgccaagacagcacactgaacttccagaacttgttttcactcactgcggggctcaccactgtggacaacaactattttgtagtacctatgaagcagaagggaacgaagcaggcagaactaagcaattactcagccctgtga

SEQ ID NO: 8 - DNA, 300 bp, Homo sapiens
atggaaaccgtgttgtttacgtttcacagatttgccggggataaaggctacttaatgaaggagggcctgaaagtactcatgggaaaggagttccttggatttttggagaatcaaaaagaccctctggctgcagacataacaatgaaggacatggaccagtgccaagacagcacactgaacttccagaacttgttttcactcactgcggggctcaccactgtggacaacaactattttgtagtacctatgaagcagaagggaacgaagcaggcagaactaagcaattactcagccctgtga

SEQ ID NO: 9 - DNA, 1143 bp, Homo sapiens
atgtcgctcatggtcgtcagcatggcgtgtgttgggttcttcttgctgcagggggcctggccacatgagggagtccacagaaaaccttccctcctggcccacccaggtcccctggtgaaatcagaagagacagtcatcctgcaatgttggtcagatgtcatgtttgagcacttccttctgcacagagaggggaagtttaatgacactttgcgcctcactggagagctccatgatggggtctccaaggccaacttctccatcggtcgcatgacgcaagaccttgcagggacctacagatgctacggttctgttcctcattccccctatcagttgtcagctcccagtgaccctctggacatcgtgattacaggtctatgtgggaaaccttctctctcagcccagccgcgccccatggttaaggcaggagagagcgtgaccttgtcctgcagctcccggagctcctatgacatctaccatctatcaagggacggggaggctcatgaacttaggttccctgcagtgcccaaggtcaatggaaccttccaggccaactttcctctgggccctgccacccacggagggacctacagatgcttcggctctttccgtgactctccctacgagtggtcagaccttagtgacccactgcttgtttctgtcacaggaaacccttctagtagttggccttcacccactgaaccaagcttcaaaactggtatccgcagacacctgcacattctgattgggacctcagtggctatcatcctcttcatcatcctcttcttctttctccttcattgctgctgctccaacaaaaagaatgctgctgtaatggaccaagagcctgccggggacagaacagtgaacagggaggactctgatgatcaagaccctcaggaggtgacatatgcacagttggatcactgcgttttcacacagacaaaaatcacttccccttctcagaggcccaagacacctccaacagataccaccatgtacatggaacttccaaatgctaagccaagatcattgtctcctgcccataagcaccacagtcaggccttgaggggatcttctagggagacaacagccctgtctcaaaaccgggttgctagctcccatgtaccagcagctggaatctga

SEQ ID NO: 10 - DNA, 1143 bp, Homo sapiens
atgtcgctcatggtcgtcagcatggcgtgtgttgggttcttcttgctgcagggggcctggccacatgagggagtccacagaaaaccttccctcctggcccacccaggtcccctggtgaaatcagaagagacagtcatcctgcaatgttggtcagatgtcatgtttgagcacttccttctgcacagagaggggaagtttaatgacactttgcgcctcactggagagctccatgatggggtctccaaggccaacttctccatcggtcgcatgacgcaagaccttgcagggacctacagatgctacggttctgttcctcattccccctatcagttgtcagctcccagtgaccctctggacatcgtgattacaggtctatgtgggaaaccttctctctcagcccagccgcgccccatggttaaggcaggagagagcgtgaccttgtcctgcagctcccggagctcctatgacatctaccatctatcaagggacggggaggctcatgaacttaggttccctgcagtgcccaaggtcaatggaaccttccaggccaactttcctctgggccctgccacccacggagggacctacagatgcttcggctctttccgtgactctccctacgagtggtcagaccttagtgacccactgcttgtttctgtcacaggaaacccttctagtagttggccttcacccactgaaccaagcttcaaaactggtatcgccagacacctgcatgctgtgattaggtactcagtggccatcatcctcttcaccatccttcccttctttctccttcatcgctggtgctccaaaaaaaaaaatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttgccagctctaatgtaccagcagctggaatctga

SEQ ID NO: 11 - DNA, 1056 bp, Homo sapiens
atggggaacctgtttatgctctgggcagctctgggcatatgctgtgctgcattcagtgcctctgcctggtcagtgaacaatttcctgataacaggtcccaaggcctatctgacctacacgactagtgtggccttgggtgcccagagtggcatcgaggagtgcaagttccagtttgcttgggaacgctggaactgccctgaaaatgctcttcagctctccacccacaacaggctgagaagtgctaccagagagacttccttcatacatgctatcagctctgctggagtcatgtacatcatcaccaagaactgtagcatgggtgacttcgaaaactgtggctgtgatgggtcaaacaatggaaaaacaggaggccatggctggatctggggaggctgcagcgacaatgtggaatttggggaaaggatctccaaactctttgtggacagtttggagaaggggaaggatgccagagccctgatgaatcttcacaacaacagggccggcagactggtggtgagagccaccatgaaaaggacatgcaaatgtcatggcatctctgggagctgcagcatacagacatgctggctgcagctggctgaattccgggagatgggagactacctaaaggccaagtatgaccaggcgctgaaaattgaaatggataagcggcagctgagagctgggaacagcgccgagggccactgggtgcccgctgaggccttccttcctagcgcagaggcggaactgatctttttagaggaatcaccagattactgtacctgcaattccagcctgggcatctatggcacagagggtcgtgagtgcctacagaacagccacaacacatccaggtgggagcgacgtagctgtgggcgcctgtgcactgagtgtgggctgcaggtggaagagaggaaaactgaggtcataagcagctgtaactgcaaattccagtggtgctgtacggtcaagtgtgaccagtgtaggcatgtggtgagcaagtattactgcgcacgctccccaggcagtgcccagtccctgggtaagggcagtgcctga

SEQ ID NO: 12 - DNA, 1089 bp, Homo sapiens
atggatccggtggcggccgaggccccgggcgaggccttcctggcgcggcgacggcctgagggcggtggcgggtccgcgcggccgcgttacagcctgttggcggagatcgggcgcggcagctacggcgtggtttatgaggcagtggccgggcgcagcggggcccgggtggcggtcaagaagatccgctgcgacgcccccgagaacgtggagctggcgctggctgaattctgggccctcaccagcctcaagcggcgccaccagaacgtcgtgcagtttgaggagtgcgtcctgcagcgcaatgggttagcccagcgcatgagtcacggcaacaagagctcgcagctttacctgcgcctggtggagacctcgctgaaaggagaaaggatcctgggttatgctgaggagccctgctatctctggtttgtcatggagttctgtgaaggtggagacctgaatcagtatgtcctgtcccggaggccagacccagccaccaacaaaagtttcatgctacagctgacgagcgccattgccttcctgcacaaaaaccatattgtgcacagggacctgaagccagacaacatcctcatcacagagcggtctggcacccccatcctcaaagtggccgactttggactaagcaaggtctgtgctgggctggcaccccgaggcaaagagggcaatcaagacaacaaaaatgtgaatgtgaataagtactggctgtcctcagcctgcggttcggacttctacatggctcctgaagtctgggagggacactacacagccaaggcggacatctttgccctgggcattatcatctgggcaatgatagaaagaatcacttttattgactctgagaccaagaaggagctcctggggacctacattaaacaggggactgagatcgtccctgttggtgaggcgctgctagaaaacccaaagatggagttgcacatcccccaaaaacgcaggacttccatgtctgaggggatcaagcagctcttgaaagatatgttagctgctaacccacaggaccggcctgatgcctttgaacttgaaaccagaatggaccaggtcacatgtgctgcttaa

SEQ ID NO: 13 - DNA, 813 bp, Homo sapiens
atgtgtggcaggttcctgcggcggctgctggcggaggagagccggcgctccacccccgtggggcgcctcttgcttcccgtgctcctgggattccgccttgtgctgctggctgccagtgggcctggagtctatggtgatgagcagagtgaattcgtgtgtcacacccagcagccgggctgcaaggctgcctgcttcgatgccttccaccccctctccccgctgcgtttctgggtcttccaggtcatcttggtggctgtacccagcgccctctatatgggtttcactctgtatcacgtgatctggcactgggaattatcaggaaaggggaaggaggaggagaccctgatccagggacgggagggcaacacagatgtcccaggggctggaagcctcaggctgctctgggcttatgtggctcagctgggggctcggcttgtcctggagggggcagccctggggttgcagtaccacctgtatgggttccagatgcccagctcctttgcatgtcgccgagaaccttgccttggtagtataacctgcaatctgtcccgcccctctgagaagaccattttcctaaagaccatgtttggagtcagcggtttctgtctcttgtttacttttttggagcttgtgcttctgggtttggggagatggtggaggacctggaagcacaaatcttcctcttctaaatacttcctaacttcagagagcaccagaagacacaagaaagcaaccgatagcctcccagtggtggaaaccaaagagcaatttcaagaagcaggtgagaaggacacgctctcttcctgtcactga

SEQ ID NO: 14 - DNA, 687 bp, Homo sapiens
atgtgtggcaggttcctgcggcggctgctggcggaggagagccggcgctccacccccgtggggcgcctcttgcttcccgtgctcctgggattccgccttgtgctgctggctgccagtgggcctggagtctatggtgatgagcagagtgaattcgtgtgtcacacccagcagccgggctgcaaggctgcctgcttcgatgccttccaccccctctccccgctgcgtttctgggtcttccaggtcatcttggtggctgtacccagcgccctctatatgggtttcactctgtatcacctgggggctcggcttgtcctggagggggcagccctggggttgcagtaccacctgtatgggttccagatgcccagctcctttgcatgtcgccgagaaccttgccttggtagtataacctgcaatctgtcccgcccctctgagaagaccattttcctaaagaccatgtttggagtcagcggtttctgtctcttgtttacttttttggagcttgtgcttctgggtttggggagatggtggaggacctggaagcacaaatcttcctcttctaaatacttcctaacttcagagagcaccagaagacacaagaaagcaaccgatagcctcccagtggtggaaaccaaagagcaatttcaagaagcaggtgagaaggacacgctctcttcctgtcactga

SEQ ID NO: 15 - DNA, 2061 bp, Homo sapiens
atgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctatgcccccaaagaccttattatcagcatttcacatgacaacacgtcagccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactaccctccacagctgctgggcccctcctgctcctgggaggctgagggtctgcactgcagctgctcctcccaggccagcccggccccctctctgcgctggtggcttggggaggagctgctggaggggaacagcagtcagggctccttcgaggtcacccccagctcagccgggccctgggccaacagctccctgagcctccatggagggctcagctccggcctcaggctccgctgtaaggcctggaacgtccacggggcccagagtggctctgtcttccagctgctaccagggaagctggagcatgggggaggacttggcctgggggctgccctgggagctggcgtcgctgccctgctcgctttctgttcctgccttgtcgtcttcagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcggagatcaagatccacacaggacagcccctgaggggcccaggctttgggcttcaattggagagggagatgtcagggatggttccaaagtga

SEQ ID NO: 16 - DNA, 2004 bp, Homo sapiens
atgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctgtgagtgtggcctgggagggtggggcgtgcagacagccccggtgggtggggaggtggaggagcccagcgggacaggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactaccctccacagctgctgggcccctcctgctcctgggaggctgagggtctgcactgcagctgctcctcccaggccagcccggccccctctctgcgctggtggcttggggaggagctgctggaggggaacagcagtcagggctccttcgaggtcacccccagctcagccgggccctgggccaacagctccctgagcctccatggagggctcagctccggcctcaggctccgctgtaaggcctggaacgtccacggggcccagagtggctctgtcttccagctgctaccagttggggatgaagaatccgagagctctagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcggagatcaagatccacacaggacagcccctgaggggcccaggctttgggcttcaattggagagggagatgtcagggatggttccaaagtga

SEQ ID NO: 17 - DNA, 300 bp, Homo sapiens
atgaagcctgaatgggctcacttttctctatggctcctcatcttgtccatgcttctccacacaccttcagggaacatatttcctccacaactcagaaaacagttacgtgaaagtggacatcttggagaatactgcaactatcctctgaagaagggcacctgtaattcatatttgaccagattctactacaacactttgacatttttatgtgagccctttgtcttcagtggctgtggaggcaacagaaacaactttaaacagaaatatttctgtgaaaaaatgtgtattactgaaaagtaa

SEQ ID NO: 18 - DNA, 774 bp, Homo sapiens
atgggcaccgtgcgcccacctcgcccctcgctcctgctggtctccacccgggagtcttgtctcttcctcctcttctgcctgcacctgggcgccgcctgcccacagccctgccggtgccctgaccacgcaggggctgtggctgtcttctgcagcttgcggggccttcaggaggtccccgaggacatcccggccaacaccgtgctcctgaagctcgatgccaacaagatctcccacctcccggacggggccttccagcacctgcaccggctcagggagctggatctgtctcacaacgccatcgaggccatcggctccgccaccttcgcgggcctggccgggggcctgcggctgctggacctgtcttacaaccgcatccagaggatccccaaggacgccctgggcaaactcagcgccaagatacgcctgtcccacaaccccctgcactgcgagtgcgccctgcaggaggccctgtgggagctgaagctggaccccgactctgtggacgagatcgcctgccacacctcagtgcaggaggagtttgtggggaagcctctggttcaggctctggatgcgggtgccagcctctgcagcgtcccccacaggaccacagacgtggccatgctggtcaccatgttcggctggttcgccatggtgatcgcctacgtcgtgtactatgtgcgccacaaccaggaggatgcccggaggcacctggagtacctgaagtctctgcccagcgcccccgcctccaaggaccccatcggcccggggccctag

SEQ ID NO: 19 - DNA, 1104 bp, Homo sapiens
atggatgacgctgctgtcctcaagcgacgaggctacctcctggggataaatttaggagagggctcctatgcaaaagtaaaatctgcttactctgagcgcctgaagttcaatgtggcgatcaagatcatcgaccgcaagaaggcccccgcagacttcttggagaaattccttccccgggaaattgagattctggccatgttaaaccactgctccatcattaagacctacgagatctttgagacatcacatggcaaggtctacatcgtcatggagctcgcagtccagggcgacctcctcgagttaatcaaaacccggggagccctgcatgaggacgaagctcgcaagaagttccaccagctttccttggccatcaagtactgccacgacctggacgtcgtccaccgggacctcaagtgtgacaaccttctccttgacaaggacttcaacatcaagctgtccgacttcagcttctccaagcgctgcctgcgggatgacagtggtcgaatggccttaagcaagaccttctgtgggtcaccagcgtatgcggccccagaggtgctgcagggcattccctaccagcccaaggtgtacgacatctggagcctaggcgtgatcctctacatcatggtctgcggctccatgccctacgacgactccaacatcaagaagatgctgcgtatccagaaggagcaccgcgtcaacttcccacgctccaagcacctgacaggcgagtgcaaggacctcatctaccacatgctgcagcccgacgtcaaccggcggctccacatcgacgagatcctcagccactgctggatgcagcccaaggcacggggatctccctctgtggccatcaacaaggagggggagagttcccggggaactgaacccttgtggacccccgaacctggctctgacaagaagtctgccaccaagctggagcctgagggagaggcacagccccaggcacagcctgagacaaaacccgaggggacagcaatgcaaatgtccaggcagtcggagatcctgggtttccccagcaagccgtcgactatggagacagaggaagggcccccccaacagcctccagagacgcgggcccagtga

SEQ ID NO: 20 - DNA, 1629 bp, Homo sapiens
atgataaattataagtcactattggcactgttgtttatattagcctcctggatcatttttacagttttccagaactccacaaaggtttggtctgctctaaacttatccatctccctccattactggaacaactccacaaagtccttattccctaaaacaccactgatatcattaaagccactaacagagactgaactcagaataaaggaaatcatagagaaactagatcagcagatcccacccagacctttcacccacgtgaacaccaccaccagcgccacacatagcacagccaccatcctcaaccctcgagatacgtactgcaggggagaccagctgcacatcctgctggaggtgagggaccacttgggacgcaggaagcaatatggcggggatttcctgagggccaggatgtcttccccagcgctgatggcaggtgcttcaggaaaggtgactgacttcaacaacggcacctacctggtcagcttcactctgttctgggagggccaggtctctctgtctctgctgctcatccaccccagtgaaggggtgtcagctctctggagtgcaaggaaccaaggctatgacagggtgatcttcactggccagtttgtcaatggcacttcccaagtccactctgaatgtggcctgatcctaaacacaaatgctgaattgtgccagtacctggacaacagagaccaagaaggcttctactgtgtgaggcctcaacacatgccctgtgctgcactcactcacatgtattctaagaacaagaaagtttcttatcttagcaaacaagaaaagagcctctttgaaaggtcaaatgtgggtgtagagattatggaaaaattcaatacaattagtgtctccaaatgcaacaagagagaaaaaatagaagagacatgccaagttggaatgaagcctcctgtccctggtggttatactttacaaggaaaatggataacaacattttgcaaccaggttcagttagacacaattaagataaatggctgtttgaaaggcaaactcatttacctcctgggagactctacactacgtcagtggatctactacttccccaaagttgtaaaaacactgaagttttttgatcttcatgaaactggaatctttaagaaacatttgcttctggatgcagaaagacacactcagattcaatggaaaaaacatagctatcccttcgtcactttccagctctactctctgatagatcatgattatatccctcgggaaattgaccggctatcaggtgacaaaaacacagccatcgtcatcacctttggccagcactttagaccatttcccattgacatttttattcgcagggccatcggtgttcaaaaggctattgaaagactgttcctaagaagcccagccactaaagtgattattaagacagaaaacatcagggagatgcacatagagacagagaggtttggagacttccatggttatattcactatcttatcatgaaggatattttcaaagacctcaacgtgggcatcattgatgcctgggacatgaccattgcatatggcactgacactatccacccacctgatcatgtgattggaaatcagattaacatgttcttaaactacatttgctaa

SEQ ID NO: 21 - DNA, 1662 bp, Homo sapiens
atgtttaggtggtttgggggaattcaggggatattcagcaatccaggaagaggagaaagtattagtaaagagcaagagctgaaagacaggaagatatcacaatccttgtcatttcagctttggtctgctctaaacttatccatctctgtccattactggaacaactccgcaaagtccttattccctaaaacatcactgataccattaaagccactaacagagactgaactcagaataaaggaaatcatagagaaactagatcagcagatcccacccagacctttcacccatgtgaacaccaccaccagtgccacacacagcacagccaccatcctcaaccctcgagatacatactgcaggggagaccagctggacatcctactggaggtgagggaccacttgggacagaggaagcaatatggtggggatttcctgagggccaggatgtcctccccagcactgacggcaggtgcttcaggaaaggtgatggacttcaacaatggcacctacctggtcagcttcactctgttctgggagggccaggtctccctgtctctgctgctcatccaccccagtgaaggggcgtcggctctctggagggcaaggaaccaaggctatgataaaattattttcaaaggcaaatttgttaatggcacctctcatgtcttcactgaatgtggcctgaccctaaactcaaatgctgaactctgtgaatatctggatgacagagaccaagaagccttctattgtatgaagcctcaacacatgccctgtgaggctctgacctacatgaccacccggaatagagaggtatcttatcttacagacaaggaaaacagccttttccacaggtccaaagtgggagttgaaatgatgaaggatcgtaaacacattgatgtcactaattgtaacaagagagaaaaaatagaagagacatgccaagttggaatgaagcctcctgtccctggtggttatactttacaaggaaaatggataacaacattttgcaaccaggttcagttagacacaattaagataaatggctgtttgaaaggcaaactcatttacctcctgggagactctacactacgtcagtggatctactacttccccaaagttgtaaaaacactgaagttttttgatcttcatgaaactggaatctttaagaaacatttgcttctggatgcagaaagacacactcagattcaatggaaaaaacatagctatcccttcgtcactttccagctctactctctgatagatcatgattatatccctcgggaaattgaccggctatcaggtgacaaaaacacagccatcgtcatcacctttggccagcactttagaccatttcccattgacatttttattcgcagggccatcggtgttcaaaaggctattgaaagactgttcctaagaagcccagccactaaagtgattattaagacagaaaacatcagggagatgcacatagagacagagaggtttggagacttccatggttatattcactatcttatcatgaaggatattttcaaagacctcaacgtgggcatcattgatgcctgggacatgaccattgcatatggcactgacactatccacccacctgatcatgtgattggaaatcagattaacatgttcttaaactacatttgctaa

SEQ ID NO: 22 - DNA, 1113 bp, Homo sapiens
atggaaggcgtggacttgctagggtttctcatcatcacattaaactgcaacgtgaccatggtgggaaagctctggttcgtcctcacgatgctgctgcggatgctggtgattgtcttggcggggcgacccgtctaccaggacgagcaggagaggtttgtctgcaacacgctgcagccgggatgcgccaatgtttgctacgacgtcttctcccccgtgtctcacctgcggttctggctgatccagggcgtgtgcgtcctcctcccctccgccgtcttcagcgtctatgtcctgcaccgaggagccacgctcgccgcgctgggcccccgccgctgccccgacccccgggagccggcctccgggcagagacgctgcccgcggccattcggggagcgcggcggcctccaggtgcccgacttttcggccggctacatcatccacctcctcctccggaccctgctggaggcagccttcggggccttgcactactttctctttggattcctggccccgaagaagttcccttgcacgcgccctccgtgcacgggcgtggtggactgctacgtgtcgcggcccacagagaagtccctgctgatgctgttcctctgggcggtcagcgcgctgtcttttctgctgggcctcgccgacctggtctgcagcctgcggcggcggatgcgcaggaggccgggaccccccacaagcccctccatccggaagcagagcggagcctcaggccacgcggagggacgccggactgacgaggagggtgggcgggaggaagagggggcaccggcgcccccgggtgcacgcgccggaggggagggggctggcagccccaggcgtacatccagggtgtcagggcacacgaagattccggatgaggatgagagtgaggtgacatcctccgccagcgaaaagctgggcagacagccccggggcaggccccaccgagaggccgcccaggaccccaggggctcaggatccgaggagcagccctcagcagcccccagccgcctggccgcgcccccttcctgcagcagcctgcagccccctgacccgcctgccagctccagtggtgctccccacctgagagccaggaagtctgagtgggtgtga

SEQ ID NO: 23 - DNA, 1071 bp, Homo sapiens
atgcccgcttcctctcttccaggaaagctctggttcgtcctcacgatgctgctgcggatgctggtgattgtcttggcggggcgacccgtctaccaggacgagcaggagaggtttgtctgcaacacgctgcagccgggatgcgccaatgtttgctacgacgtcttctcccccgtgtctcacctgcggttctggctgatccagggcgtgtgcgtcctcctcccctccgccgtcttcagcgtctatgtcctgcaccgaggagccacgctcgccgcgctgggcccccgccgctgccccgacccccgggagccggcctccgggcagagacgctgcccgcggccattcggggagcgcggcggcctccaggtgcccgacttttcggccggctacatcatccacctcctcctccggaccctgctggaggcagccttcggggccttgcactactttctctttggattcctggccccgaagaagttcccttgcacgcgccctccgtgcacgggcgtggtggactgctacgtgtcgcggcccacagagaagtccctgctgatgctgttcctctgggcggtcagcgcgctgtcttttctgctgggcctcgccgacctggtctgcagcctgcggcggcggatgcgcaggaggccgggaccccccacaagcccctccatccggaagcagagcggagcctcaggccacgcggagggacgccggactgacgaggagggtgggcgggaggaagagggggcaccggcgcccccgggtgcacgcgccggaggggagggggctggcagccccaggcgtacatccagggtgtcagggcacacgaagattccggatgaggatgagagtgaggtgacatcctccgccagcgaaaagctgggcagacagccccggggcaggccccaccgagaggccgcccaggaccccaggggctcaggatccgaggagcagccctcagcagcccccagccgcctggccgcgcccccttcctgcagcagcctgcagccccctgacccgcctgccagctccagtggtgctccccacctgagagccaggaagtctgagtgggtgtga

SEQ ID NO: 24 - DNA, 2586 bp, Homo sapiens
atggccatgagatccgggaggcacccctcgctgctgctgcttctagtgctgctgctgtggctgctgcaggtaagctctccttttgttgagacagggtctggcttcatcatccaggctggagtgcagtggcatgatcaaggctcacgtgcacgccaccatgcccggctagtttttgtatttttattagagacagggtttcaccatgttggccaggctggtctcaaactcctgacctcaagtgatctgcctgcctcagcctcccaaagtgttaggatgacaggcttgagacactgcgccctgccaagttttataatgatatgtcactataagttccaggaaaagatctaccagcctctacggcgatccaagagaagatgggttatcaccaccttggagctggaggaggaagacccgggaccctttcccaaactcattggtgagctgttcaataatatgtcttataacatgtcactaatgtatctaatcagtggacctggtgtggatgaatatccagagattggtttgttttctctagaagatcatgagaacggaaggatatatgttcaccgccctgtcgatcgagaaatgacaccatctttcacggtttattttgatgttgtggagcgctcaacaggaaaaattgtggatacatccttgattttcaacattaggatcagtgatgtgaatgatcatgcaccccagtttccagagaaggaatttaacatcactgtgcaagaaaaccaatctgcagggcaacctatttttcagatgttagcagtcgatttggatgaagaaaacactccaaattctcaagtcctttacttcctcatttctcaaacaccattactgaaagaaagtggtttccgggttgatcgccttagtggagaaatacgactctctggctgcttagattatgagaccgctcctcagtttacactgctaatcagagccagggactgtggagaaccgtcactgtcatccacgaccaccgttcacgtggatgtgcaagaaggcaacaaccacaggcctgcatttacccaggagaactataaggttcagattcctgaaggccgagccagccagggcgtgttgcgtctcctggttcaagatcgagattctccatttacatcagcttggagagcaaaattcaacatattgcatggcaatgaagaggggcattttgacatttcgactgaccctgagaccaacgaagggatattaaatgttatcaagcctttggattatgagactcgcccagcgcaaagcctcatcattgtcgtggagaatgaggagaggctcgtcttctgtgagagaggaaagcttcagccgccaaggaaggcagcagccagcgccactgtgagtgtgcaggtgacagacgccaacgacccaccagcctttcacccccagagcttcattgtcaataaagaggagggcgccaggcctgggaccctgttgggaacttttaatgccatggatccagacagccagataagatatgaactggttcatgacccagcaaattgggtcagcgtcgacaaaaactccggagtggtcatcaccgtggagccaattgaccgagaatcccctcatgtaaataacagtttttatgtaatcatcattcacgctgttgatgatggcttcccaccgcagactgctacagggaccctaatgctcttcctgtctgacatcaatgacaacgtcccgactctccggccacgttcccgctacatggaggtctgtgagtctgctgtgcatgagcccctccacatcgaggcagaggatccggacctggagccgttctctgacccatttacatttgaattggacaatacctggggaaatgcggaggacacatggaagttggggagaaattggggtcaatcagttgaacttttaaccttgagaagcctgccacgtggtaattacttggtgccactcttcattggagacaaacagggactttcccagaagcaaactgtccatgtaaggatctgcccctgtgccagtgggctcacatgtgtggagcttgcagatgcagaagtggggcttcatgtgggggccctgttccctgtctgtgcagcatttgtggctctggcagtggctctgctttttctgttgcgatgctattttgtgcttgaacctaagaggcatggatgctctgtatccaatgatgaaggccaccaaacactggtcatgtataatgcggagagcaaaggcacttcagcccagacatggtcagatgttgaaggccagaggccggctctgctcatctgcacagctgcagcaggacccacgcagggagttaaggcttacccagatgccacaatgcacagacaactcctggctccggtggaaggaaggatggcagagacattgaatcagaaactccatgttgccaatgtgctggaagatgaccccggctacctacctcacgtctacagcgaggaaggggagtgtggaggggccccatccctcagctctctggccagcttggaacaggagttgcaacctgatttgctggactctttgggttcaaaagcgactccgtttgaggaaatatattcagagtcaggtgttccttcctaa

SEQ ID NO: 25 - DNA, 351 bp, Homo sapiens
atgagagaatgcaggccaggtgtcagcacagggcctttacatgaagtaaggtgtcactataagttccaggaaaagatctaccagcctctacggcgatccaagagaagatgggttatcaccaccttggagctggaggaggaagacccgggaccctttcccaaactcattggtgagctgttcaataatatgtcttataacatgtcactaatgtatctaatcagtggacctggtgtggatgaatatccagagattggtttgttttctctagaagatcatgagaacggaaggatatatgttcaccgccctgtcgatcgagaaatgacaccatctttcacggtatctaaaacttga

SEQ ID NO: 26 - DNA, 2769 bp, Homo sapiens
atggccatgagatccgggaggcacccctcgctgctgctgcttctagtgctgctgctgtggctgctgcaggtaagctctccttttgttgagacagggtctggcttcatcatccaggctggagtgcagtggcatgatcaaggctcacgtgcacgccaccatgcccggctagtttttgtatttttattagagacagggtttcaccatgttggccaggctggtctcaaactcctgacctcaagtgatctgcctgcctcagcctcccaaagtgttaggatgacaggcttgagacactgcgccctgccaagttttataatgatatgtcactataagttccaggaaaagatctaccagcctctacggcgatccaagagaagatgggttatcaccaccttggagctggaggaggaagacccgggaccctttcccaaactcattggtgagctgttcaataatatgtcttataacatgtcactaatgtatctaatcagtggacctggtgtggatgaatatccagagattggtttgttttctctagaagatcatgagaacggaaggatatatgttcaccgccctgtcgatcgagaaatgacaccatctttcacggtttattttgatgttgtggagcgctcaacaggaaaaattgtggatacatccttgattttcaacattaggatcagtgatgtgaatgatcatgcaccccagtttccagagaaggaatttaacatcactgtgcaagaaaaccaatctgcagggcaacctatttttcagatgttagcagtcgatttggatgaagaaaacactccaaattctcaagtcctttacttcctcatttctcaaacaccattactgaaagaaagtggtttccgggttgatcgccttagtggagaaatacgactctctggctgcttagattatgagaccgctcctcagtttacactgctaatcagagccagggactgtggagaaccgtcactgtcatccacgaccaccgttcacgtggatgtgcaagaaggcaacaaccacaggcctgcatttacccaggagaactataaggttcagattcctgaaggccgagccagccagggcgtgttgcgtctcctggttcaagatcgagattctccatttacatcagcttggagagcaaaattcaacatattgcatggcaatgaagaggggcattttgacatttcgactgaccctgagaccaacgaagggatattaaatgttatcaagcctttggattatgagactcgcccagcgcaaagcctcatcattgtcgtggagaatgaggagaggctcgtcttctgtgagagaggaaagcttcagccgccaaggaaggcagcagccagcgccactgtgagtgtgcaggtgacagacgccaacgacccaccagcctttcacccccagagcttcattgtcaataaagaggagggcgccaggcctgggaccctgttgggaacttttaatgccatggatccagacagccagataagatatgaactggttcatgacccagcaaattgggtcagcgtcgacaaaaactccggagtggtcatcaccgtggagccaattgaccgagaatcccctcatgtaaataacagtttttatgtaatcatcattcacgctgttgatgatggcttcccaccgcagactgctacagggaccctaatgctcttcctgtctgacatcaatgacaacgtcccgactctccggccacgttcccgctacatggaggtctgtgagtctgctgtgcatgagcccctccacatcgaggcagaggatccggacctggagccgttctctgacccatttacatttgaattggacaatacctggggaaatgcggaggacacatggaagttggggagaaattggggtcaatcagttgaacttttaaccttgagaagcctgccacgtggtaattacttggtgccactcttcattggagacaaacagggactttcccagaagcaaactgtccatgtaaggatctgcccctgtgccagtgggctcacatgtgtggagcttgcagatgcagaagtggggcttcatgtgggggccctgttccctgtctgtgcagcatttgtggctctggcagtggctctgctttttctgttgcgatgctattttgtgcttgaacctaagaggcatggatgctctgtatccaatgatgaaggccaccaaacactggtcatgtataatgcggagagcaaaggcacttcagcccagacatggtcagatgttgaaggccagaggccggctctgctcatctgcacagctgcagcaggacccacgcagggagttaaggcttacccagatgccacaatgcacagacaactcctggctccggtggaaggaaggatggcagagacattgaatcagtctaaagagagaaatcgcttcagcctcagcaggggctgcatcatcccccagggaagagccacagctgggcgaggattgccacaagacatttacaaggagatgatgccacggagactaacgcagactggtaaacggaaacacggggctttggctcgaacaccctctttcaagaaagttgtttatgaccacaaggaagatgaggagaacaaggctgggagaaagcaaaggagccacttgtttaaggttatgcagctgagaaatgaacaaggtggggttcgagtccagagtgcgcattccccatcaccccttaacaaaaaggcatgtttcccaggagactacagaggagaatcggcaggtggtcacaattgcagggctgtctcaggctga

SEQ ID NO: 27 - DNA, 16908 bp, Homo sapiens
atgatttcctgggaagttgtccatacagtattcctgtttgctcttctttattcttccctagctcaagatgcgagcccccagtcagagatcagagctgaggaaattcccgagggggcctccacgttggcttttgtgtttgatgtgactggttctatgtatgatgatttagttcaggtgattgaaggggcttccaaaattttggagacgtctttgaaaagacctaaaagacctcttttcaactttgcgttggtgcctttccatgatccagaaattggcccagtgacaattaccacagatcccaagaaatttcaatatgaactcagagaactgtatgttcagggtggtggtgattgcccagaaatgagtattggagctataaaaattgccttggaaatttctcttcctggttctttcatctatgttttcactgatgctcggtccaaagattaccggctcacccatgaggtgctgcaacttatccaacagaaacagtcacaagtcgtatttgttctgactggagattgtgatgacaggacccatattggatataaagtctatgaagaaattgcctctacaagttctggtcaagtgttccatctggacaaaaaacaagttaatgaggtattaaaatgggtagaagaagcagtacaggcctccaaagttcaccttttatccacagatcatttggaacaggctgtaaatacttggagaattccttttgatcccagcctgaaagaggtcactgtgtctttgagtgggccttctccaatgattgaaattcgcaatcctttagggaagctgataaaaaagggatttggcctgcatgagctattaaatatccataactctgccaaagtagtgaatgtgaaagagccagaggctggaatgtggacagtgaagacctcaagcagtggaaggcactctgttcgcattactggcctcagtactattgatttccgagctggcttttctcgaaagcccaccctggacttcaaaaaaacagtcagcagaccagtgcaaggaatacctacctatgtactgctcaatacttctggaatttccactccagctagaatagatcttcttgaacttttgagtatctcaggaagttctcttaagactattcctgttaaatattacccacatcgaaaaccttatggcatatggaatatttctgactttgtaccaccaaatgaagctttctttctcaaagtaacaggctatgataaagatgattacctcttccagagagtatcaagtgtttccttttctagtattgtcccagatgctcccaaagttacgatgcctgagaaaaccccaggatactatctgcagccgggccaaattccctgctctgttgacagtcttttgccctttaccttgagctttgtcagaaatggagttacacttggagtagaccagtatttgaaagaatctgccagtgtgaacttagatattgcaaaggtcactttgtctgacgaaggtttctatgaatgcattgctgtcagcagtgcaggtactggacgggcacagacattttttgacgtatcagagccccctccggtcatccaagtgcctaacaatgttacagtcactcctggagagagagcagttttaacatgtctcatcatcagtgcggtggattacaatctaacctggcagaggaatgacagagatgtcagactggcagagccagcgagaattaggaccttggctaatctgtcattggagctaaagagtgtgaaattcaacgatgctggagagtatcattgtatggtttctagtgaaggtggatcatcagccgcttcagttttcctcacagtgcaagaaccacccaaagtcactgtgatgcccaagaatcagtctttcacaggagggtctgaggtctccatcatgtgttctgcaacaggttatcccaaaccaaagattgcctggaccgttaacgatatgtttatcgtgggttcacacaggtataggatgacctcagatggtaccttatttatcaaaaatgcagctcccaaagatgcagggatctatggttgcctagcaagtaattcagctggaacagataaacagaattctactctcagatacattgaagcccctaagttgatggtagttcagagtgagctcttggttgcccttggggatataaccgttatggaatgcaaaacctctggtattcctccacctcaagttaaatggttcaaaggagatcttgagttgaggccctcaacattcctcattattgaccctctcttgggacttttgaagattcaagaaacacaagatctggatgctggcgattatacctgtgtagccatcaatgaggctggaagagcaactggcaagataactctggatgttggctcacctccagttttcatacaagaacctgctgatgtgtctatggaaattggctcaaatgtgacattaccttgttatgttcagggttatccagaaccaacaatcaaatggcgaagattagacaacatgccaattttctcaagacctttttcagttagttccatcagccaactaagaacaggagctctctttattttaaacttatgggcaagtgataaaggaacctatatttgtgaagctgaaaaccagtttggaaagatccagtcagagacaacagtaacagtgaccggacttgttgctccacttattggaatcagcccttcagtggccaatgttattgaaggacagcagcttactttgccctgtactctgttagctggaaatcccattccagaacgtcggtggattaagaattcagctatgttgctccaaaatccttacatcactgtgcgcagtgatgggagcctccatattgaaagagttcagcttcaggatggtggtgaatatacttgtgtggccagtaacgttgctgggaccaataacaaaactacctctgtggttgtgcatgttctgccaaccattcagcatgggcagcagatactcagtacaattgaaggcattccagtaactttaccatgcaaagcaagtggaaatcccaaaccgtctgtcatctggtccaagaaaggagagctgatttcaaccagcagtgctaagttttcagcaggagctgatggtagtctgtatgtggtatcacctggaggagaggagagtggggagtatgtctgcactgccaccaatacagccggctacgccaaaaggaaagtgcagctaacagtctatgtaaggcccagagtgtttggagatcaacgaggactgtcccaggataagcctgttgagatctccgtccttgcaggggaagaggtaacacttccatgtgaagtgaagagcttacctccacccataattacttgggccaaagaaacccagctcatctcaccgttctctccaagacacacattcctcccttctggttcaatgaagatcactgaaacccgcacttcagatagtgggatgtatctttgtgttgccacaaatattgctgggaatgtgactcaggctgtcaaattaaatgtccatgttcctccaaagatacagcgtggacctaaacatctcaaagtccaagttggtcaaagagtggatattccatgtaatgctcaagggactcctcttcctgtaatcacctggtccaaaggtggaagcactatgctggttgatggagagcaccatgttagcaatccagacggaactttaagcatcgaccaagccacgccctcagatgctggcatatatacatgtgttgctactaacatagcaggcactgatgaaacagagataacgctacatgtccaagaaccacccacagtggaagatctagaacctccatataacactactttccaagaaagagtggccaatcaacgcattgaatttccatgtcctgcaaaaggtacccctaaaccaaccatcaaatggttacacaatggtagagagttgacaggcagagagcctggcatttctatcttggaagatggcacattgctggttattgcttctgttacaccctatgacaatggggagtacatctgtgtggcagtcaatgaagctggaaccacagaaagaaaatataacctcaaagtccatgttcctccagtaattaaagataaagaacaagttacaaatgtgtcggtgttgttaaatcagctgaccaatctcttctgtgaagtggaaggcactccatctcccatcattatgtggtataaagataatgtccaggtgactgaaagcagcactattcagactgtgaacaatgggaagatactgaagctcttcagagccactccagaggatgcaggaagatattcctgcaaagcaattaatattgcaggcacttctcagaagtactttaacattgatgtgctagttccacccaccataataggtaccaacttcccaaatgaagtctcagttgtcctcaaccgtgacgtcgcccttgaatgccaggtcaaaggcactccctttcctgatattcattggttcaaagatggcaagcctttatttttgggcgatcctaatgttgaacttctagacagaggacaagtcttacatttaaagaatgcacggagaaatgacaaggggcgctaccaatgtactgtgtctaatgcagctggcaaacaagccaaggatataaaactgactatctataatcctcctagtattaaaggaggaaatgtcaccacagacatatcagtattgatcaacagccttattaaactggaatgtgaaacacggggacttccaatgcctgccattacttggtataaggacgggcagccaatcatgtccagctcacaagcactttatattgataaaggacaatatcttcatattcctcgagcacaggtctctgattcagcaacatatacgtgtcacgtagccaatgttgctggaactgctgaaaaatcattccatgtggatgtctatgttcctccaatgattgaaggcaacttggccacgcctttgaataagcaagtagttattgctcattctctgacactggagtgcaaagctgctggaaacccttctcccattctcacctggttgaaagatggtgtacctgtgaaagctaatgacaatatccgcatagaagctggtgggaagaaactcgaaatcatgagtgcccaagaaattgatcgaggacagtacatatgcgtggctaccagtgtggcaggagaaaaggaaatcaaatatgaagttgatgtcttggtgccaccagctatagaaggaggagatgaaacatcttacttcattgtgatggttaataacttactggagctagattgtcatgtgacaggctctcccccaccaactatcatgtggctgaaggatggccagttaattgatgaaagggatggattcaagattttattaaatggacgcaaactggttattgctcaggctcaagtgtcaaacacaggcctttatcggtgcatggcagcaaatactgctggagaccacaagaaggaatttgaagtgactgttcatgttcctccaacaatcaagtcctcaggcctttctgagagagttgtggtaaaatacaagcctgtcgccttgcagtgcatagccaatgggattccaaatccttccattacatggttaaaagatgaccagcctgtgaacactgcccaaggaaaccttaaaatacagtcttctggtcgagttctacaaattgccaaaaccctgttggaagatgctggcagatacacatgtgtggctaccaacgcagctggagaaacacaacagcacattcaactgcatgttcatgaaccacctagtctggaagatgctggaaaaatgctgaatgagactgtgttggtgagcaaccctgtacagctggagtgtaaggcagctggaaatcctgtgcctgttattacatggtacaaagataatcgtctactctcaggttccaccagcatgactttcttgaacagaggacagatcattgatattgaaagtgcccagatctcagatgctggcatatataaatgcgtggccatcaactcagctggagctacagagttattttacagtctgcaagttcatgtggccccatcaatttctggcagcaataacatggtggcagtggtggttaataacccggtgaggttagaatgtgaagccagaggtattcctgccccaagtctgacctggttgaaagatgggagtcctgtttctagtttttctaatggattacaggttctctctggtggtcgaatcctagcattgaccagtgcacaaatcagcgacacaggaaggtacacctgcgtggcagtgaatgctgctggagaaaagcaaagggacattgacctccgagtatatgttccgccaaatattatgggagaagaacagaatgtctctgtcctcattagccaagctgtggaattactatgtcaaagtgatgctattcccccacctactcttacttggttaaaagacggccaccccttgctgaagaaaccaggcctcagtatatctgaaaatagaagtgtgttaaagattgaagatgctcaggttcaagacactggtcgttacacttgtgaagcaacaaatgttgctggaaaaactgaaaaaaactacaatgtcaacatttgggtccccccaaatattggtggttctgatgaacttactcaacttacagtcattgaagggaatctcattagtctgttgtgtgaatcaagtggtattccacccccaaatctcatctggaagaagaaaggctctccagtgctgactgattccatggggcgagttagaattttatctgggggcaggcaattacaaatttcaattgctgaaaagtctgatgcagcactctattcatgtgtggcgtcgaatgttgctgggactgcaaagaaagaatacaatctgcaagtttacattagaccaaccataaccaacagtggcagccaccctactgaaattattgtgacccgagggaagagtatctccttggagtgtgaggtgcagggtattccaccaccaacagtgacctggatgaaagatggccaccccttgatcaaggcaaagggagtagaaatactggatgaaggtcacatccttcagctgaagaacattcatgtatctgacacaggccgttatgtgtgtgttgctgtgaatgtagcaggaatgactgacaaaaaatatgacttaagtgtccatgctcctccaagcatcataggaaaccacaggtcacctgaaaatattagtgtggtagaaaagaactcagtatctttgacttgtgaagcttctggaattcccctgccttccataacctggttcaaagatgggtggcctgtcagccttagcaattctgtgaggattctttcaggaggcaggatgctacggctgatgcagaccacaatggaagatgctggccaatatacttgcgttgtaaggaatgcagctggtgaagaaagaaaaatctttgggctttcagtattagtaccacctcatattgtgggtgaaaatacattggaagatgtgaaggtaaaagagaaacagagtgttacgctgacttgtgaagtgacagggaatccagtgccagaaattacatggcacaaagatgggcagcccctccaagaagatgaagcccatcacattatatctggtggccgttttcttcaaattaccaatgtccaggtgccacacactggaagatatacatgtttggcttccagtccagctggccacaagagcaggagcttcagtcttaatgtatttgtatctcctacaattgctggtgtaggtagtgatggcaaccctgaagatgtcactgtcatccttaacagccctacatctttggtctgtgaagcttattcatatcctccagctaccatcacctggtttaaggatggcactcctttagaatctaaccgaaatattcgtattcttccaggaggcagaactctgcagatcctcaatgcacaggaggacaatgctggaagatactcttgtgtagccacgaatgaggctggagaaatgataaagcactatgaagtgaaggtgtacattccacccataatcaataaaggggacctttgggggccaggtctttcccctaaagaagtgaagatcaaagtaaacaacactctgaccttggaatgtgaagcgtatgcaattccttctgcctccctcagctggtacaaggatggacagccccttaaatccgatgatcatgttaatattgctgcgaatggacacacacttcaaataaaggaggctcaaatatcagacaccggacgatatacttgtgtagcatctaacattgcaggtgaagatgagttggattttgatgtgaatattcaagttcctccaagttttcagaaactctgggaaataggaaacatgctagatactggcaggaatggtgaagccaaagatgtgatcatcaacaatcccatttctctttactgtgagacaaatgctgctccccctcctacactgacatggtacaaagatggccaccctctgacctcaagtgataaagtattgattttgccaggagggcgagtgttgcagattcctcgggctaaagtagaagatgctgggagatacacatgtgtggctgtgaatgaggctggagaagattcccttcaatatgatgtccgtgtactcgtgccgccaattatcaagggagcaaatagtgatctccctgaagaggtcaccgtgctggtgaacaagagtgcactgatagagtgtttatccagtggcagcccagcaccaaggaattcctggcagaaagatggacagcccttgctagaagatgaccatcataaatttctatctaatggacgaattctgcagattctgaatactcaaataacagatatcggcaggtatgtgtgtgttgctgagaacacagctgggagtgccaaaaaatattttaacctcaatgttcatgttcctccaagtgtcattggtcctaaatctgaaaatcttaccgtcgtggtgaacaatttcatctctttgacctgtgaggtctctggttttccacctcctgacctcagctggctcaagaatgaacagcccatcaaactgaacacaaatactctcattgtgcctggtggtcgaactctacagattattcgggccaaggtatcagatggtggtgaatacacttgtatagctatcaatcaagctggcgaaagcaagaaaaagttttccctgactgtttatgtgcccccaagcattaaagaccatgacagtgaatctctttctgtagttaatgtaagagagggaacttctgtgtctttggagtgtgagtcgaacgctgtgccacctccagtcatcacttggtataagaatgggcggatgataacagagtctactcatgtggagattttagctgatggacaaatgctacacattaagaaagctgaggtatctgacacaggccagtatgtatgtagagctataaatgtagcaggacgggatgataaaaatttccacctcaatgtatatgtgccacccagtattgaaggacctgaaagagaagtgattgtggagacgatcagcaatcctgtgacattaacatgtgatgccactgggatcccacctcccacgatagcatggttaaagaaccacaagcgcatagaaaattctgactcactggaagttcgtattttgtctggaggtagcaaactccagattgcccggtctcagcattcagatagtggaaactatacatgtattgcttcaaatatggagggaaaagcccagaaatattactttctttcaattcaagttcctccaagtgttgctggtgctgaaattccaagtgatgtcagtgtccttctaggagaaaatgttgagctggtctgcaatgcaaatggcattcctactccacttattcaatggcttaaagatggaaagcccatagctagtggtgaaacagaaagaatccgagtgagtgcaaatggcagcacattaaacatttatggagctcttacatctgacacggggaaatacacatgtgttgctactaatcccgctggagaagaagaccgaatttttaacttgaatgtctatgttacacctacaattaggggtaataaagatgaagcagagaaactaatgactttagtggatacttcaataaatattgaatgcagagccacagggacgcctccaccacagataaactggctgaagaatggacttcctctgcctctctcctcccatatccggttactggcagcaggacaagttatcaggattgtgagagctcaggtgtctgatgtcgctgtgtatacttgtgtggcctccaacagagctggggtggataataagcattacaatcttcaagtgtttgcaccaccaaatatggacaattcaatggggacagaggaaatcacagttctcaaaggtagttccacctctatggcatgcattactgatggaaccccagctcccagtatggcctggcttagagatggccagcctctggggcttgatgcccatctgacagtcagcacccatggaatggtcctgcagctcctcaaagcagagactgaagattcgggaaagtacacctgcattgcctcaaatgaagctggagaagtcagcaagcactttatcctcaaggtcctagaaccacctcacattaatggatctgaagaacatgaagagatatcagtaattgttaataacccacttgaacttacctgcattgcttctggaatcccagcccctaaaatgacctggatgaaagatggccggccccttccacagacggatcaagtgcaaactctaggaggaggagaggttcttcgaatttctactgctcaggtggaggatacaggaagatatacatgtctggcatccagtcctgcaggagatgatgataaggaatatctagtgagagtgcatgtacctcctaatattgctggaactgatgagccccgggatatcactgtgttacggaacagacaagtgacattggaatgcaagtcagatgcagtgcccccacctgtaattacttggctcagaaatggagaacggttacaggcaacacctcgagtgcgaatcctatctggagggagatacttgcaaatcaacaatgctgacctaggtgatacagccaattatacctgtgttgccagcaacattgcaggaaagactacaagagaatttattctcactgtaaatgttcctccaaacataaaggggggcccccagagccttgtaattcttttaaataagtcaactgtattggaatgcatcgctgaaggtgtgccaactccaaggataacatggagaaaggatggagctgttctagctgggaatcatgcaagatattccatcttggaaaatggattccttcatattcaatcagcacatgtcactgacactggacggtatttgtgtatggccaccaatgctgctggaacagatcgcaggcgaatagatttacaggtccatgttcctccatctattgctccgggtcctaccaacatgactgtaatagtaaatgttcaaactactctggcttgtgaggctactgggataccaaaaccatcaatcaattggagaaaaaatgggcatcttcttaatgtggatcaaaatcagaactcatacaggctcctttcttcaggttcactagtaattatttccccttctgtggatgacactgcaacctatgaatgtactgtgacaaacggtgctggagatgataaaagaactgtggatctcactgtccaagttccaccttccatagctgatgagcctacagatttcctagtaaccaaacatgccccagcagtaattacctgcactgcttcgggagttccatttccctcaattcactggaccaaaaatggtataagactgcttcccaggggagatggctatagaattctgtcctcaggagcaattgaaatacttgccacccaattaaaccatgctggaagatacacttgtgtcgctaggaatgcggctggctctgcacatcgacacgtgacccttcatgttcatgagcctccagtcattcagccccaaccaagtgaactacacgtcattctgaacaatcctattttattaccatgtgaagcaacagggacacccagtcctttcattacttggcaaaaagaaggcatcaatgttaacacttcaggcagaaaccatgcagttcttcctagtggcggcttacagatctccagagctgtccgagaggatgctggcacttacatgtgtgtggcccagaacccggctggtacagccttgggcaaaatcaagttaaatgtccaagttcctccagtcattagccctcatctaaaggaatatgttattgctgtggacaagcccatcacgttatcctgtgaagcagatggcctccctccgcctgacattacatggcataaagatgggcgtgcaattgtggaatctatccgccagcgcgtcctcagctctggctctctgcaaatagcatttgtccagcctggtgatgctggccattacacgtgcatggcagccaatgtagcaggatcaagcagcacaagcaccaagctcaccgtccatgtaccacccaggatcagaagtacagaaggacactacacggtcaatgagaattcacaagccattcttccatgcgtagctgatggaatccccacaccagcaattaactggaaaaaagacaatgttcttttagctaacttgttaggaaaatacactgctgaaccatatggagaactcattttagaaaatgttgtgctggaggattctggcttctatacctgtgttgctaacaatgctgcaggtgaagatacacacactgtcagcctgactgtgcatgttctccccacttttactgaacttcctggagacgtgtcattaaataaaggagaacagctacgattaagctgtaaagctactggtattccattgcccaaattaacatggaccttcaataacaatattattccagcccactttgacagtgtgaatggacacagtgaacttgttattgaaagagtgtcaaaagaggattcaggtacttatgtgtgcaccgcagagaacagcgttggctttgtgaaggcaattggatttgtttatgtgaaagaacctccagtcttcaaaggtgattatccttctaactggattgaaccacttggtgggaatgcaatcctgaattgtgaggtgaaaggagaccccaccccaaccatccagtggaacagaaagggagtggatattgaaattagccacagaatccggcaactgggcaatggctccctggccatctatggcactgttaatgaagatgccggtgactatacatgtgtagctaccaatgaagctggggtggtggagcgcagcatgagtctgactctgcaaagtcctcctattatcactcttgagccagtggaaactgttattaatgctggtggcaaaatcatattgaattgtcaggcaactggagagcctcaaccaaccattacatggtcccgtcaagggcactctatttcctgggatgaccgggttaacgtgttgtccaacaactcattatatattgctgatgctcagaaagaagatacctctgaatttgaatgtgttgctcgaaacttaatgggttctgtccttgtcagagtgccagtcatagtccaggttcatggtggattttcccagtggtctgcatggagagcctgcagtgtcacctgtggaaaaggcatccaaaagaggagtcgtctgtgcaaccagccccttccagccaatggtgggaagccctgccaaggttcagatttggaaatgcgaaactgtcaaaataagccttgtccagtggatggtagctggtcggaatggagtctttgggaagaatgcacaaggagctgtggacgcggcaaccaaaccaggaccaggacttgcaataatccatcagttcagcatggtgggcggccatgtgaagggaatgctgtggaaataattatgtgcaacattaggccttgcccagttcatggagcatggagcgcttggcagccttggggaacatgcagcgaaagttgtgggaaaggtactcagacaagagcaagactttgtaataacccaccaccagcgtttggtgggtcctactgtgatggagcagaaacacagatgcaagtttgcaatgaaagaaattgtccaattcatggcaagtgggcgacttgggccagttggagtgcctgttctgtgtcatgtggaggaggtgccagacagagaacaaggggctgctccgaccctgtgccccagtatggaggaaggaaatgcgaagggagtgatgtccagagtgatttttgcaacagtgacccttgcccaacccatggtaactggagtccttggagtggctggggaacatgcagccggacgtgtaacggagggcagatgcggcggtaccgcacatgtgataaccctcctccctccaatgggggaagagcttgtgggggaccagactcccagatccagaggtgcaacactgacatgtgtcctgtggatggaagttggggaagctggcatagttggagccagtgctctgcctcctgtggaggaggtgaaaagactcggaagcggctgtgcgaccatcctgtgccagttaaaggtggccgtccctgtcccggagacactactcaggtgaccaggtgcaatgtacaagcatgtccaggtgggccccagcgagccagaggaagtgttattggaaatattaatgatgttgaatttggaattgctttccttaatgccacaataactgatagccctaactctgatactagaataatacgtgccaaaattaccaatgtacctcgtagtcttggttcagcaatgagaaagatagtttctattctaaatcccatttattggacaacagcaaaggaaataggagaagcagtcaatggctttaccctcaccaatgcagtcttcaaaagagaaactcaagtggaatttgcaactggagaaatcttgcagatgagtcatattgcccggggcttggattccgatggttctttgctgctagatatcgttgtgagtggctatgtcctacagcttcagtcacctgctgaagtcactgtaaaggattacacagaggactacattcaaacaggtcctgggcagctgtacgcctactcaacccggctgttcaccattgatggcatcagcatcccatacacatggaaccacaccgttttctatgatcaggcacagggaagaatgcctttcttggttgaaacacttcatgcatcctctgtggaatctgactataaccagatagaagagacactgggttttaaaattcatgcttcaatatccaaaggagatcgcagtaatcagtgcccctccgggtttaccttagactcagttggacctttttgtgctgatgaggatgaatgtgcagcagggaatccctgctcccatagctgccacaatgccatggggacttactactgctcctgccctaaaggcctcaccatagctgcagatggaagaacttgtcaagatattgatgagtgtgctttgggtaggcatacctgccacgctggtcaggactgtgacaatacgattggatcttatcgctgtgtggtccgttgtggaagtggctttcgaagaacctctgatgggctgagttgtcaagatattaatgaatgtcaagaatccagcccctgtcaccagcgctgtttcaatgccataggaagtttccattgtggatgtgaacctgggtatcagctcaaaggcagaaaatgcatggatgtgaacgagtgtagacaaaatgtatgcagaccagatcagcactgtaagaacacccgtggtggctataagtgcattgatctttgtccaaatggaatgaccaaggcagaaaatggaacctgtattgatattgatgaatgtaaagatgggacccatcagtgcagatataaccagatatgtgagaatacaagaggcagctatcgttgtgtatgcccaagaggttatcggtctcaaggagttggaagaccctgcatggacattaatgaatgtgaacaagtgcctaaaccttgtgcacatcagtgctccaacacccccggcagcttcaagtgtatctgtccaccaggacaacatttattaggggacgggaaatcttgcgctggattggagaggctgccaaattatggcactcaatacagtagctataaccttgcacggttctcccctgtgagaaacaactatcaacctcaacagcattacagacagtactcacatctctacagctcctactcagagtatagaaacagcagaacatctctctccaggactagaaggactattaggaaaacttgccctgaaggctctgaggcaagccatgacacatgtgtagatattgatgaatgtgaaaatacagatgcctgccagcatgagtgtaagaatacctttggaagttatcagtgcatctgcccacctggctatcaactcacacacaatggaaagacatgccaagatatcgatgaatgtctggagcagaatgtgcactgtggacccaatcgcatgtgcttcaacatgagaggaagctaccagtgcatcgatacaccctgtccacccaactaccaacgggatcctgtttcagggttctgcctcaagaactgtccacccaatgatttggaatgtgccttgagcccatatgccttggaatacaaactcgtctccctcccatttggaatagccaccaatcaagatttaatccggctggttgcatacacacaggatggagtgatgcatcccaggacaactttcctcatggtagatgaggaacagactgttccttttgccttgagggatgaaaacctgaaaggagtggtgtatacaacacgaccactacgagaagcagagacctaccgcatgagggtccgagcctcatcctacagtgccaatgggaccattgaatatcagaccacattcatagtttatatagctgtgtccgcctatccatactaa

SEQ ID NO: 28 - DNA, 2889 bp, Homo sapiens
atggagcatggagcgcttggcagccttggggaacatgcagcgaaagttgtgggaaaggtactcagacaagagcaagactttgtaataacccaccaccagcgtttggtgggtcctactgtgatggagcagaaacacagatgcaagtttgcaatgaaagaaattgtccaattcatggcaagtgggcgacttgggccagttggagtgcctgttctgtgtcatgtggaggaggtgccagacagagaacaaggggctgctccgaccctgtgccccagtatggaggaaggaaatgcgaagggagtgatgtccagagtgatttttgcaacagtgacccttgcccaagtgagtgttggaaatacccatggtaactggagtccttggagtggctggggaacatgcagccggacgtgtaacggagggcagatgcggcggtaccgcacatgtgataaccctcctccctccaatgggggaagagcttgtgggggaccagactcccagatccagaggtgcaacactgacatgtgtcctgtggatggaagttggggaagctggcatagttggagccagtgctctgcctcctgtggaggaggtgaaaagactcggaagcggctgtgcgaccatcctgtgccagttaaaggtggccgtccctgtcccggagacactactcaggtgaccaggtgcaatgtacaagcatgtccaggtgggccccagcgagccagaggaagtgttattggaaatattaatgatgttgaatttggaattgctttccttaatgccacaataactgatagccctaactctgatactagaataatacgtgccaaaattaccaatgtacctcgtagtcttggttcagcaatgagaaagatagtttctattctaaatcccatttattggacaacagcaaaggaaataggagaagcagtcaatggctttaccctcaccaatgcagtcttcaaaagagaaactcaagtggaatttgcaactggagaaatcttgcagatgagtcatattgcccggggcttggattccgatggttctttgctgctagatatcgttgtgagtggctatgtcctacagcttcagtcacctgctgaagtcactgtaaaggattacacagaggactacattcaaacaggtcctgggcagctgtacgcctactcaacccggctgttcaccattgatggcatcagcatcccatacacatggaaccacaccgttttctatgatcaggcacagggaagaatgcctttcttggttgaaacacttcatgcatcctctgtggaatctgactataaccagatagaagagacactgggttttaaaattcatgcttcaatatccaaaggagatcgcagtaatcagtgcccctccgggtttaccttagactcagttggacctttttgtgctgatgaggatgaatgtgcagcagggaatccctgctcccatagctgccacaatgccatggggacttactactgctcctgccctaaaggcctcaccatagctgcagatggaagaacttgtcaagatattgatgagtgtgctttgggtaggcatacctgccacgctggtcaggactgtgacaatacgattggatcttatcgctgtgtggtccgttgtggaagtggctttcgaagaacctctgatgggctgagttgtcaagatattaatgaatgtcaagaatccagcccctgtcaccagcgctgtttcaatgccataggaagtttccattgtggatgtgaacctgggtatcagctcaaaggcagaaaatgcatggatgtgaacgagtgtagacaaaatgtatgcagaccagatcagcactgtaagaacacccgtggtggctataagtgcattgatctttgtccaaatggaatgaccaaggcagaaaatggaacctgtattgatattgatgaatgtaaagatgggacccatcagtgcagatataaccagatatgtgagaatacaagaggcagctatcgttgtgtatgcccaagaggttatcggtctcaaggagttggaagaccctgcatggacattaatgaatgtgaacaagtgcctaaaccttgtgcacatcagtgctccaacacccccggcagcttcaagtgtatctgtccaccaggacaacatttattaggggacgggaaatcttgcgctggattggagaggctgccaaattatggcactcaatacagtagctataaccttgcacggactagaaggactattaggaaaacttgccctgaaggctctgaggcaagccatgacacatgtgtagatattgatgaatgtgaaaatacagatgcctgccagcatgagtgtaagaatacctttggaagttatcagtgcatctgcccacctggctatcaactcacacacaatggaaagacatgccaagatatcgatgaatgtctggagcagaatgtgcactgtggacccaatcgcatgtgcttcaacatgagaggaagctaccagtgcatcgatacaccctgtccacccaactaccaacgggatcctgtttcagggttctgcctcaagaactgtccacccaatgatttggaatgtgccttgagcccatatgccttggaatacaaactcgtctccctcccatttggaatagccaccaatcaagatttaatccggctggttgcatacacacaggatggagtgatgcatcccaggacaactttcctcatggtagatgaggaacagactgttccttttgccttgagggatgaaaacctgaaaggagtggtgtatacaacacgaccactacgagaagcagagacctaccgcatgagggtccgagcctcatcctacagtgccaatgggaccattgaatatcagaccacattcatagtttatatagctgtgtccgcctatccatactaa

SEQ ID NO: 29 - DNA, 3096 bp, Homo sapiens
atgctcttctacacaggactgaagtgtggaattcgcatggtcaacatgaaaagtaaggaacctgccgtgggatctagattcttctctagaattagtagttggagaaattcaacagtgactggacatccatggcaggtctccctaaaatcagatgagcaccacttctgtggaggaagcttgattcaagaagatcgggttgttacagcagcacactgcctggacagcctcagtgagaagcagctgaagaatataactgtgacttctggggagtacagcctctttcagaaggataagcaagaacagaatattcctgtctcaaaaattattacccatcctgaatacaacagccgtgaatatatgagtcctgatattgcactgctgtatctaaaacacaaagtcaagtttggaaatgctgttcagccaatctgtcttcctgacagcgatgataaagttgaaccaggaattctttgcttatccagtggatggggcaagatttccaaaacatcagaatattcaaatgtcctacaagaaatggaacttcccatcatggatgacagagcgtgtaatactgtgctcaagagcatgaacctccctcccctgggaaggaccatgctgtgtgctggcttccctgattggggaatggacgcctgccagggggactctggaggaccactggtttgtagaagaggtggtggaatctggattcttgctgggataacttcctgggtagctggttgtgctggaggttcagttcccgtaagaaacaaccatgtgaaggcatcacttggcattttctccaaagtgtctgagttgatggattttatcactcaaaacctgttcacagattgtaaacctcaggggacagtgttatttggagaaagtgggaagatttgttacccccattccaaaggagactactattctcataattgcttatatgtttggaaaataatggtaccagaagataaaataatcctgataaaatttacaagtttagacatggaaaagcaagttggatgtgatcatgactatgtatctttacgatcaagcagtggagtgctttttagtaaggtctgtggaaaaatattgccttcaccattgctggcagagaccagtgaggccatggttccatttgtttctgatacagaagacagtggcagtggctttgagcttaccgttactgctgtacagaagtcagaagcagggtcaggttgtgggagtctggctatattggtagaagaagggacaaatcactctgccaagtatcctgatttgtatcccagtaacacaaggtgtcattggttcatttgtgctccagagaagcacattataaagttgacatttgaggactttgctgtcaaatttagtccaaactgtatttatgatgctgttgtgatttacggtgattctgaagaaaagcacaagttagctaaactttgtggaatgttgaccatcacttcaatattcagttctagtaacatgacggtgatatactttaaaagtgatggtaaaaatcgtttacaaggcttcaaggccagatttaccattttgccctcagagtctttaaacaaatttgaaccaaagttacctccccaaaacaatcctgtatctaccgtaaaagctattctgcatgatgtctgtggcatccctccatttagtccccagtggctttccagaagaatcgcaggaggggaagaagcctgcccccactgttggccatggcaggtgggtctgaggtttctaggcgattaccaatgtggaggtgccatcatcaacccagtgtggattctgaccgcagcccactgtgtgcaattgaagaataatccactctcctggactattattgctggggaccatgacagaaacctgaaggaatcaacagagcaggtgagaagggccaaacacataatagtgcatgaagactttaacacactaagttatgactctgacattgccctaatacaactaagctctcctctggagtacaactcggtggtgaggccagtatgtctcccacacagcgcagagcctctattttcctcggagatctgtgctgtgaccggatggggaagcatcagtgcagatggtggcctagcaagtcgcctacagcagattcaagtgcatgtgttagaaagagaggtctgtgaacacacttactattctgcccatccaggagggatcacagagaagatgatctgtgctggctttgcagcatctggagagaaagatttctgccagggagactctggtgggccactagtatgtagacatgaaaatggtccctttgtcctctatggcattgtcagctggggagctggctgtgtccagccatggaagccgggtgtatttgccagagtgatgatcttcttggactggatccaatcaaaaatcaatggtaaattgttttcaaatgttattaaaacaataacctctttctttagagtgggtttgggaacagtgagttgttgctctgaagcagagctagaaaagcctagaggcttttttcccacaccacggtatctactggattatagaggaagactggaatgttcttgggtgctcagagtttcagcaagcagtatggcaaaatttaccattgagtatctgtcactcctggggtctcctgtgtgtcaagactcagttctaattatttatgaagaaagacacagtaagagaaagacggcaggtggattacatggaagaagactttactcaatgactttcatgagtcctggaccgctggtgagggtgacattccatgcccttgtacgaggtgcatttggtataagctatattgtcttgaaagtcctaggtccaaaggacagtaaaataaccagactttcccaaagttcaaacagagagcacttggtcccttgtgaggatgttcttctgaccaagccagaagggatcatgcagatcccaagaaattctcacagaactactatgggttgccaatggagattagtagcccctttaaatcacatcattcagcttaatattattaacttcccgatgaagccaacaacttttgtctgtcatggtcatctgcgtgtttacgaaggatttggaccaggaaaaaaattaataggtaactga

SEQ ID NO: 30 - DNA, 135 bp, Homo sapiens
atgtctgacaaacccgatatggctgagattgagaaattcgataagtcgaaactgaagaagacagagatgcaagagaaaaatccactgccttccaaagaaacgattgaacaggagaagcaagcaagcgaatcgtaa

SEQ ID NO: 31 - DNA, 1323 bp, Homo sapiens
atggcagaaaataatgaaaatattagtaaaaatgtagatgtaaggcccaaaactagtcggagcagaagtgccgacagaaaagacggttatgtgtggagtggaaagaagttatcttggtcaaaaaagagtgagagttattcagatgctgagacagtgaatggtatagagaaaaccgaagtgtctttaaggaaccaagaaaggaagcacagctgttcatccattgagttggacttagatcattcctgtgggcatcgatttttaggccgatctcttaaacagaaactgcaagatgccgtggggcagtgttttccaataaagaattgtagtagtcggcactcttcagggcttccgtctaaaaggaaaattcatatcagtgaactcatgttagataagtgtcctttcccacctcgatcagatttagcctttaggtggcattttattaaacgacacactgctcctataaattccaaatcagatgaatgggtaagcacagacttgtctcagactgaattgagggatggtcagctaaaacgaagaaatatggaagaaaatataaactgtttctcacataccaatgttcagccctgtgtcataaccaccgacaatgctttgtgtagagaaggtcctatgactggctctgtgatgaacctggtttcaaataacagtatagaagatagtgatatggattccgatgatgaaattctaacactttgcacaagttccagaaaaagaaacaaacccaaatgggatttggatgatgaaatcctgcagttggaaacacctcctaaataccacacgcagattgattatgtccactgtcttgtaccagacctccttcagatcaataacaacccatgttactggggagtgatggataaatacgcagccgaagcactactggaaggaaaaccagagggtacctttttacttcgagactcagcacaggaagactatttattctctgttagttttagacgctatagtcgttctcttcatgctagaattgaacagtggaatcacaactttagctttgatgcacatgacccctgtgtcttccattctcctgacattactgggctcctagaacattataaggacccaagcgcctgtatgttctttgaaccacttctatccactcccttaattcggactttccctttttccctgcagcatatatgcagaacagttatttgtaactgtacaacttatgatggcatcgatgcccttccaattccttcttctatgaaattatatctgaaggaatatcattataaatcaaaagttagagtactcaggattgatgcaccagaacagcaatgctag

SEQ ID NO: 32 - DNA, 1779 bp, Homo sapiens
atgacctgctggctgtgcgtcctgagcctgcccctgctcctgctgcccgcggcgccgcccccggctggaggctgcccggcccgctgcgagtgcaccgtgcagacccgcgcggtggcctgcacgcgccgccgcctgaccgccgtgcccgacggcatcccggccgagacccgcctgctggagctcagccgcaaccgcatccgctgcctgaacccgggcgacctggccgcgctgcccgcgctggaggagctggacctgagcgagaacgccatcgcgcacgtggagcccggcgccttcgccaacctgccgcgcctgcgcgtcctgcgtctccgtggcaaccagctgaagctcatcccgcccggggtcttcacgcgcctggacaacctcacgctgctggacctgagcgagaacaagctggtaatcctgctggactacactttccaggacctgcacagcctgcgccggctggaagtgggcgacaacgacctggtattcgtctcgcgccgcgccttcgcggggctgctggccctggaggagctgaccctggagcgctgcaacctcacggctctgtccggggagtcgctgggccatctgcgcagcctgggcgccctgcggctgcgccacctggccatcgcctccctggaggaccagaacttccgcaggctgcccgggctgctgcacctggagattgacaactggccgctgctggaggaggtggcggcgggcagcctgcggggcctgaacctgacctcgctgtcggtcacccacaccaacatcaccgccgtgccggccgccgcgctgcggcaccaggcgcacctcacctgcctcaatctgtcgcacaaccccatcagcacggtgccgcgggggtcgttccgggacctggtccgcctgcgcgagctgcacctggccggggccctgctggctgtggtggagccgcaggccttcctgggcctgcgccagatccgcctgctcaacctctccaacaacctgctctccacgttggaggagagcaccttccactcggtgaacacgctagagacgctgcgcgtggacgggaacccgctggcctgcgactgtcgcctgctgtggatcgtgcagcgtcgcaagaccctcaacttcgacgggcggctgccggcctgcgccaccccggccgaggtgcgcggcgacgcgctgcgaaacctgccggactccgtgctgttcgagtacttcgtgtgccgcaaacccaagatccgggagcggcggctgcagcgcgtcacggccaccgcgggcgaagacgtccgcttcctctgccgcgccgagggcgagccggcgcccaccgtggcctgggtgaccccccagcaccggccggtgacggccaccagcgcgggccgggcgcgcgtgctccccggggggacgctggagatccaggacgcgcggccgcaggacagcggcacctacacgtgcgtggccagcaacgcgggcggcaacgacacctacttcgccacgctgaccgtgcgccccgagccggccgccaaccggaccccgggcgaggcccacaacgagacgctggcggccctgcgcgcgccgctcgacctcaccaccatcctggtgtccaccgccatgggctgcatcaccttcctgggcgtggtcctcttctgcttcgtgctgctgttcgtgtggagccgcggccgcgggcagcacaaaaacaacttctcggtggagtactccttccgcaaggtggatgggccggccgccgcggcgggccagggaggcgcgcgcaagttcaacatgaagatgatctga

SEQ ID NO: 33 - DNA, 1710 bp, Homo sapiens
atgaagaagaagttagtggtgctgggcctgctggccgtggtcctggtgctggtcattgtcagcctctgtctctggctgccctcggcctccaaggaacctgacaaccatgtgtacaccagggctgccgtggccgcggatgccaagcagtgcttggagattgggagggacacactgcgggacggtggctctgcagtggatgcagccattgcagccctgttgtgtgtggggctcatgaatgcccacagcatgggcatcggggttggcctcttcctcaccatctacaacagcaccactcgaaaagctgaggtcatcaatgcccgcgaggtggcccccaggctggcctttgccagcatgttcaacagctcggagcagtcccagaagggagggctgtcggtggcggtgcctggggagatccgaggctatgagctggcacaccagcggcatgggcggctgccctgggctcgcctcttccagcccagcatccagctggcccgccagggcttccccgtgggcaagggcttggcggcagtcctggaaaacaagcggaccgtcatcgagcagcagcctgtcttgtgtgaggtgttctgccgggatagaaaggtgctttgggagggggagagactgaccctgccgcggctggctgacacctacgagatgctggccatcgagggtgcccaggccttctacaacggcagcctcatggcccagattgtgaaggacatccaggcggctgggggcattgtgacagctgaggacctgaacaactactgtgctgagctgatcgagcacccgctgaacatcagcctgggagacgcggtgctgtacatgcccagtgcgcggctcagcgggcccgtgctggccctcatcctcaacatcctcaaagggtacaacttctcccgggagagcgtggagacccccgagcagaagggcctgacgtactaccgcatcgtagaggctttccggtttgcctacgccaagaggaccctgcttggggaccccaagtttgtggatgtgactgaggtggtccgcaacatgacctctgagttcttcactgcccagctccggtcccagatctctgaccacaccactcacccgatctcctactacaagcccgagttctacacgccggatgacgggggcactgctcacctgtctgtcgtcgcagaggacggcagtgctgtgtccgccaccagcaccatcaacctctactttggctccaaggtctgctccccggtcagtgggatcctgttcaataatgaatggacgacttcagctctcccagcattcaccaatgagtttggggcacccccctcacctgccaatttcatccagccagggaagcagccgctcttgtccatgtgcccgacgatcatggtgggccaggacggccaggtccggatggtggtgggagctgctgggggcacgcagatcaccacagacactgcactggccatcatctacaacctctggttcggctatgacgtgaagagggccgtggaggagccccggctgcacaacaagcttctgcccaacgtcacgacagtggagagaaacattgaccaggcagtgactgcagccctggagacccggcaccatcacacccagatcgcgtccaccttcatcgctgtggtgcaagccatcgtccgcacggctggtggctgggcagctgcctcggactccaggaaaggcggggaacctgctggctactga

SEQ ID NO: 34 - DNA, 468 bp, Homo sapiens
atgtttgagcctcagtgggtggatagggaccaggctgggccaggcaaggtcgggtgctgtctgacctggctgggcggtagctttggctccaaggtctgctccccggtcagtgggatcctgttcaataatgaatggacgacttcagctctcccagcattcaccaatgagtttggggcacccccctcacctgccaatttcatccagccagggaagcagccgctcttgtccatgtgcccgacgatcatggtgggccaggacggccaggtccggatggtggtgggagctgctgggggcacgcagatcaccacagacactgcactggcagtgactgcagccctggagacccggcaccatcacacccagatcgcatccaccttcatcgctgtggtgcaagccatcgtccgcacggctggtggctgggcagctgcctcggactccaggaaaggcggggaacctgctggctactga

SEQ ID NO: 35 - DNA, 1155 bp, Homo sapiens
atggtggctgaggctggttcaatgccggctgcctcctctgtgaagaagccatttggtctcagaagcaagatgggcaagtggtgccgccactgcttcccctggtgcagggggagcggcaagagcaacgtgggcacttctggagaccacgacgattctgctatgaagacactcaggagcaagatgggcaagtggtgccgccactgcttcccctggtgcagggggagcagcaagagcaacgtgggcacttctggagaccacgacgactctgctatgaagacactcaggagcaagatgggcaagtggtgctgccactgcttcccctgctgcagggggagcggcaagagcaaagtgggcccttggggagactacgacgacagcgctttcatggagccgaggtaccacgtccgtcgagaagatctggacaagctccacagagctgcctggtggggtaaagtccccagaaaggatctcatcgtcatgctcaaggacactgacatgaacaagaaggacaagcaaaagaggactgctctacatctggcctctgccaatggaaattcagaagtagtaaaactcctgctggacagacgatgtcaacttaatatccttgacaacaaaaagaggacagctctgacaaaggccgtacaatgccgggaagatgaatgtgcgttaatgttgctggaacatggcactgatccgaatattccagatgagtatggaaataccgctctacactatgctatctacaatgaagataaattaatggccaaagcactgctcttatacggtgctgatatcgaatcaaaaaacaagcatggcctcacaccactgttacttggtgtacatgagcaaaaacagcaagtggtgaaattcttaatcaagaaaaaagcaaatttaaatgcactggatagatatggaagaactgctctcatacttgctgtatgttgtggatcggcaagtatagtcagccttctacttgagcaaaacattgatgtatcttctcaagatctatctggacagacggccagagagtatgctgtttctagtcatcataatgtaatttgccagttactttctgactacaaagaaaaacagatgctaaaagtctcttctgaaaacagcaatccaggaaatgtctcaagaaccagaaataaataa

SEQ ID NO: 36 - DNA, 3066 bp, Homo sapiens
atggctttgcggggcgccgcgggagcgaccgacaccccggtgtcctcggccgggggagcccccggcggctcagcgtcctcgtcgtccacctcctcgggcggctcggcctcggcgggcgcggggctgtgggccgcgctctatgactacgaggctcgcggcgaggacgagctgagcctgcggcgcggccagctggtggaggtgctgtcgcaggacgccgccgtgtcgggcgacgagggctggtgggcaggccaggtgcagcggcgcctcggcatcttccccgccaactacgtggctccctgccgcccggccgccagccccgcgccgccgccctcgcggcccagctccccggtacacgtcgccttcgagcggctggagctgaaggagctcatcggcgctgggggcttcgggcaggtgtaccgcgccacctggcagggccaggaggtggccgtgaaggcggcgcgccaggacccggagcaggacgcggcggcggctgccgagagcgtgcggcgcgaggctcggctcttcgccatgctgcggcaccccaacatcatcgagctgcgcggcgtgtgcctgcagcagccgcacctctgcctggtgctggagttcgcccgcggcggagcgctcaaccgagcgctggccgctcgccgcatccctccgcacgtgctggtcaactgggccgtgcagatagcgcggggcatgctctacctgcatgaggaggccttcgtgcccatcctgcaccgggacctcaagtccagcaacattttgctacttgagaagatagaacatgatgacatctgcaataaaactttgaagattacagattttgggttggcgagggaatggcacaggaccaccaaaatgagcacagcaggcacctatgcctggatggcccccgaagtgatcaagtcttccttgttttctaagggaagcgacatctggagctatggagtgctgctgtgggaactgctcaccggagaagtcccctatcggggcattgatggcctcgccgtggcttatggggtagcagtcaataaactcactttgcccattccatccacctgccctgagccgtttgccaagctcatgaaagaatgctggcaacaagaccctcatattcgtccatcgtttgccttaattctcgaacagttgactgctattgaaggggcagtgatgactgagatgcctcaagaatcttttcattccatgcaagatgactggaaactagaaattcaacaaatgtttgatgagttgagaacaaaggaaaaggagctgcgatcccgggaagaggagctgactcgggcggctctgcagcagaagtctcaggaggagctgctaaagcggcgtgagcagcagctggcagagcgcgagatcgacgtgctggagcgggaacttaacattctgatattccagctaaaccaggagaagcccaaggtaaagaagaggaagggcaagtttaagagaagtcgtttaaagctcaaagatggacatcgaatcagtttaccttcagatttccagcacaagataaccgtgcaggcctctcccaacttggacaaacggcggagcctgaacagcagcagttccagtcccccgagcagccccacaatgatgccccgactccgagccatacagttgacttcagatgaaagcaataaaacttggggaaggaacacagtctttcgacaagaagaatttgaggatgtaaaaaggaattttaagaaaaaaggttgtacctggggaccaaattccattcaaatgaaagatagaacagattgcaaagaaaggataagacctctctccgatggcaacagtccttggtcaactatcttaataaaaaatcagaaaaccatgcccttggcttcattgtttgtggaccagccagggtcctgtgaagagccaaaactttcccctgatggattagaacacagaaaaccaaaacaaataaaattgcctagtcaggcctacattgatctacctcttgggaaagatgctcagagagagaatcctgcagaagctgaaagctgggaggaggcagcctctgcgaatgctgccacagtctccattgagatgactcctacgaatagtctgagtagatccccccagagaaagaaaacggagtcagctctgtatgggtgcaccgtccttctggcatcggtggctctgggactggacctcagagagcttcataaagcacaggctgctgaagaaccgttgcccaaggaagagaagaagaaacgagagggaatcttccagcgggcttccaagtcccgcagaagcgccagtcctcccacaagcctgccatccacctgtggggaggccagcagcccaccctccctgccactgtcaagtgccctgggcatcctctccacaccttctttctccacaaagtgcctgctgcagatggacagtgaagatccactggtggacagtgcacctgtcacttgtgactctgagatgctcactccggatttttgtcccactgccccaggaagtggtcgtgagccagccctcatgccaagacttgacactgattgtagtgtatcaagaaacttgccgtcttccttcctacagcagacatgtgggaatgtaccttactgtgcttcttcaaaacatagaccgtcacatcacagacggaccatgtctgatggaaatccgaccccaactggtgcaactattatctcagccactggagcctctgcactgccactctgcccctcacctgctcctcacagtcatctgccaagggaggtctcacccaagaagcacagcactgtccacatcgtgcctcagcgtcgccctgcctccctgagaagccgctcagatctgcctcaggcttacccacagacagcagtgtctcagctggcacagactgcctgtgtagtgggtcgcccaggaccacatcccacccaattcctcgctgccaaggagagaactaaatcccatgtgccttcattactggatgctgacgtggaaggtcagagcagggactacactgtgccactgtgcagaatgaggagcaaaaccagccggccatctatatatgaactggagaaagaattcctgtcttaa

SEQ ID NO: 37 - DNA, 1155 bp, Homo sapiens
atggtggctgaggtttgttcaatgcccgctgcctctgctgtgaagaagccatttgatctcaggagcaagatgggcaagtggtgccaccaccgcttcccctgctgcagggggagcggcaagagcaacatgggcacttctggagaccacgacgactcctttatgaagacgctcaggagcaagatgggcaagtgttgccaccactgcttcccctgctgcagggggagcggcacgagcaatgtgggcacttctggagaccatgacaactcctttatgaagacactcaggagcaagatgggcaagtggtgctgtcactgcttcccctgctgcagggggagcggcaagagcaacgtgggcacttggggagactacgacgacagcgccttcatggagccgaggtaccacgtccgtcgagaagatctggacaagctccacagagctgcctggtggggtaaagtccccagaaaggatctcatcgtcatgctcagggacactgacatgaacaagagggacaagcaaaagaggactgctctacatttggcctctgccaatggaaattcagaagtagtacaactcctgctggacagacgatgtcaacttaacgtccttgacaacaaaaaaaggacagctctgataaaggccgtacaatgccaggaagatgaatgtgtgttaatgttgctggaacatggcgctgatggaaatattcaagatgagtatggaaataccgctctacactatgctatctacaatgaagataaattaatggccaaagcactgctcttatatggtgctgatattgaatcaaaaaacaagtgtggcctcacaccacttttgcttggcgtacatgaacaaaaacagcaagtggtgaaatttttaatcaagaaaaaagctaatttaaatgcacttgatagatatggaagaactgccctcatacttgctgtatgttgtggatcagcaagtatagtcaatcttctacttgagcaaaatgttgatgtatcttctcaagatctatctggacagacggccagagagtatgctgtttctagtcatcatcatgtaatttgtgaattactttctgactataaagaaaaacagatgctaaaaatctcttctgaaaacagcaatccagaaaatgtctcaagaaccagaaataaataa

SEQ ID NO: 38 - DNA, 1011 bp, Homo sapiens
atgtgggcaacctgctgcaactggttctgcctggatggacagcctgaggaggtcccaccaccccagggagccaggatgcaggcctattccaaccctgggtacagctccttcccttccccaacaggcttggaaccaagctgcaagtcctgtggggctcactttgcaaacacggccaggaagcagcagacctgcttggactgtaagaaaaatttttgcatgacctgttcgagccaagtagggaatgggccccgcctctgccttctctgccaacggtttcgagctacagcctttcagcgagaggagctcatgaagatgaaggtgaaggacttgagggactatctcagcctccatgacatctctaccgaaatgtgccgggagaaagaagagctggtgctcttggtccttggccagcagcctgtaatctcccaggaggacaggactcgtgcctccaccttgtccccagactttcctgagcagcaggccttcctgacccagcctcactccagcatggttccacctacctcacccaacctcccctcttcatctgcacaagccacctctgttcccccagcccaggttcaggagaatcagcagtctattgactcagaggacagctttgtcccaggccgaagggcctctctgtctgacctgactgacctggaggacattgaaggcctgacagtgcggcagctgaaagagatcttggctcgcaactttgtcaactacaagggctgctgtgagaagtgggagctgatggagagagtgacccggctatacaaggatcagaaaggactccagcacctggtcagtggtgccgaagaccaaaacgggggagcagtaccatcaggcttggaggagaacctgtgtaagatctgcatggactcacccattgactgtgttcttctggagtgtggccacatggtaacctgtaccaagtgtggcaagcgcatgaatgaatgtcccatctgccggcagtatgtaatccgagctgtgcatgtcttccggtcctga

SEQ ID NO: 39 - DNA, 1071 bp, Homo sapiens
atgtgggcaacctgctgcaactggttctgcctggatggacagcctgaggaggtcccaccaccccagggagccaggatgcaggcctattccaaccctgggtacagctccttcccttccccaacaggcttggaaccaagctgcaagtcctgtggggctcactttgcaaacacggccaggaagcagcagacctgcttggactgtaagaaaaatttttgcatgacctgttcgagccaagtagggaatgggccccgcctctgccttctctgccaacggtttcgagctacagcctttcagcgagaggagctcatgaagatgaaggtgaaggacttgagggactatctcagcctccatgacatctctaccgaaatgtgccgggagaaagaagagctggtgctcttggtccttggccagcagcctgtaatctcccaggaggacaggactcgtgcctccaccttgtccccagactttcctgagcagcaggccttcctgacccagcctcactccagcatggttccacctacctcacccaacctcccctcttcatctgcacaagccacctctgttcccccagcccaggttcaggagaatcagcaggccaatggccatgtgtctcaggatcaagaggaacccgtctacctggagagcgtggccagagtacctgctgaggatgagacccagtctattgactcagaggacagctttgtcccaggccgaagggcctctctgtctgacctgactgacctggaggacattgaaggcctgacagtgcggcagctgaaagagatcttggctcgcaactttgtcaactacaagggctgctgtgagaagtgggagctgatggagagagtgacccggctatacaaggatcagaaaggactccagcacctggggggagcagtaccatcaggcttggaggagaacctgtgtaagatctgcatggactcacccattgactgtgttcttctggagtgtggccacatggtaacctgtaccaagtgtggcaagcgcatgaatgaatgtcccatctgccggcagtatgtaatccgagctgtgcatgtcttccggtcctga

SEQ ID NO: 40 - DNA, 1728 bp, Homo sapiens
atgagtgggaattgggtgcacccaggccagatcctaatttgggctatctgggtccttgcagcccccaccaaagggccttctgctgaagggccacagaggaacaccaggctgggatggattcagggcaagcaagtcactgtgctgggaagccctgtgcctgtgaacgtgttcctcggagtcccctttgctgctcccccgctgggatccctgcgatttacgaacccgcagcctgcatcgccctgggataacttgcgagaagccacctcctaccctaatttgtgcctccagaactcagagtggctgctcttagatcaacatatgctcaaggtgcattacccgaaattcggagtgtcagaagactgcctctacctgaacatctatgcgcctgcccacgccgatacaggctccaagctccccgtcttggtgtggttcccaggaggtgccttcaagactggctcagcctccatctttgatgggtccgccctggctgcctatgaggacgtgctggttgtggtcgtccagtaccggctaggaatatttggtttcttcaccacatgggatcagcatgctccggggaactgggccttcaaggaccaggtggctgctctgtcctgggtccagaagaacatcgagttcttcggtggggaccccagctctgtgaccatctttggcgagtccgcgggagccataagtgtttctagtcttatactgtctcccatggccaaaggcttattccacaaagccatcatggagagtggggtggccatcatcccttacctggaggcccatgattatgagaagagtgaggacctgcaggtggttgcacatttctgtggtaacaatgcgtcagactctgaggccctgctgaggtgcctgaggacaaaaccctccaaggagctgctgaccctcagccagaaaacaaagtctttcactcgagtggttgatggtgctttctttcctaatgagcctctagatctattgtctcagaaagcatttaaagcaattccttccatcatcggagtcaataaccacgagtgtggcttcctgctgcctatgaaggaggctcctgagatcctcagtggctccaacaagtcccttgccctccatctgatacaaaacatcctgcacatcccgcctcagtatttgcaccttgtggctaatgaatacttccatgacaagcactccctgactgaaatccgagacagtcttctggacttgcttggagatgtgttctttgtggtccctgcactgatcacagctcgatatcacagagatgctggtgcacctgtctacttctatgagtttcggcaccggcctcagtgctttgaagacacgaagccagcttttgtcaaagccgaccacgctgatgaagtccgctttgtgttcggtggtgccttcctgaagggggacattgttatgttcgaaggagccacggaggaggagaagttactgagccggaagatgatgaaatactgggctacctttgctcgaaccgggaatcctaatgggaacgacctgtctctgtggccagcttataatctgactgagcagtacctccagctggacttgaacatgagcctcggacagagactcaaagaaccgcgggtggatttttggaccagcaccatccccctgatcctgtctgcctccgacatgctccacagtcctctttcttccttaactttcctctctctcctccagcctttctttttcttttgtgctccttga

SEQ ID NO: 41 - DNA, 1746 bp, Homo sapiens
atgagtgggaattgggtgcacccaggccagatcctaatttgggctatctgggtccttgcagcccccaccaaagggccttctgctgaagggccacagaggaacaccaggctgggatggattcagggcaagcaagtcactgtgctgggaagccctgtgcctgtgaacgtgttcctcggagtcccctttgctgctcccccgctgggatccctgcgatttacgaacccgcagcctgcatcgccctgggataacttgcgagaagccacctcctaccctaatttgtgcctccagaactcagagtggctgctcttagatcaacatatgctcaaggtgcattacccgaaattcggagtgtcagaagactgcctctacctgaacatctatgcgcctgcccacgccgatacaggctccaagctccccgtcttggtgtggttcccaggaggtgccttcaagactggctcagcctccatctttgatgggtccgccctggctgcctatgaggacgtgctggttgtggtcgtccagtaccggctaggaatatttggtttcttcaccacatgggatcagcatgctccggggaactgggccttcaaggaccaggtggctgctctgtcctgggtccagaagaacatcgagttcttcggtggggaccccagctctgtgaccatctttggcgagtccgcgggagccataagtgtttctagtcttatactgtctcccatggccaaaggcttattccacaaagccatcatggagagtggggtggccatcatcccttacctggaggcccatgattatgagaagagtgaggacctgcaggtggttgcacatttctgtggtaacaatgcgtcagactctgaggccctgctgaggtgcctgaggacaaaaccctccaaggagctgctgaccctcagccagaaaacaaagtctttcactcgagtggttgatggtgctttctttcctaatgagcctctagatctattgtctcagaaagcatttaaagcaattccttccatcatcggagtcaataaccacgagtgtggcttcctgctgcctatggtaagaattctggctgtccatactgccactccctcaaaccgtgatgcagctttggcttcaacagctgggcatttccacagaagacatcagcacatcccgcctcagtatttgcaccttgtggctaatgaatacttccatgacaagcactccctgactgaaatccgagacagtcttctggacttgcttggagatgtgttctttgtggtccctgcactgatcacagctcgatatcacagagatgctggtgcacctgtctacttctatgagtttcggcaccggcctcagtgctttgaagacacgaagccagcttttgtcaaagccgaccacgctgatgaagtccgctttgtgttcggtggtgccttcctgaagggggacattgttatgttcgaaggagccacggaggaggagaagttactgagccggaagatgatgaaatactgggctacctttgctcgaaccgggaatcctaatgggaacgacctgtctctgtggccagcttataatctgactgagcagtacctccagctggacttgaacatgagcctcggacagagactcaaagaaccgcggagagatgtgtgggtgacggggtatcctcagccatggaaagctgccatcatccagaataaaaaacctagaagtcaaattctaggcatcaagggtcggatcagcaatgccaagaagaaatga

SEQ ID NO: 42 - DNA, 1071 bp, Homo sapiens
atggaagatgatttatcctggaggtcttggtcacttctctgcatggagatactgtctcccatggccaaaggcttattccacaaagccatcatggagagtggggtggccatcatcccttacctggaggcccatgattatgagaagagtgaggacctgcaggtggttgcacatttctgtggtaacaatgcgtcagactctgaggccctgctgaggtgcctgaggacaaaaccctccaaggagctgctgaccctcagccagaaaacaaagtctttcactcgagtggttgatggtgctttctttcctaatgagcctctagatctattgtctcagaaagcatttaaagcaattccttccatcatcggagtcaataaccacgagtgtggcttcctgctgcctatgaaggaggctcctgagatcctcagtggctccaacaagtcccttgccctccatctgatacaaaacatcctgcacatcccgcctcagtatttgcaccttgtggctaatgaatacttccatgacaagcactccctgactgaaatccgagacagtcttctggacttgcttggagatgtgttctttgtggtccctgcactgatcacagctcgatatcacagagatgctggtgcacctgtctacttctatgagtttcggcaccggcctcagtgctttgaagacacgaagccagcttttgtcaaagccgaccacgctgatgaagtccgctttgtgttcggtggtgccttcctgaagggggacattgttatgttcgaaggagccacggaggaggagaagttactgagccggaagatgatgaaatactgggctacctttgctcgaaccgggaatcctaatgggaacgacctgtctctgtggccagcttataatctgactgagcagtacctccagctggacttgaacatgagcctcggacagagactcaaagaaccgcgggtggatttttggaccagcaccatccccctgatcctgtctgcctccgacatgctccacagtcctctttcttccttaactttcctctctctcctccagcctttctttttcttttgtgctccttga

SEQ ID NO: 43 - DNA, 1776 bp, Homo sapiens
atggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgagtatttagaacttcgatttaacaaatgtgttcgtctctgtggaacagtcctcttcattgttcaaacaattctgtatactggaattgttatttatgcccctgccctggctttgaatcaagtcacaggatttgatctgtggggcgcggtagtggcaacgggggtggtctgcacattctactgcacactgggtggtcttaaagcagttatctggacagatgtttttcaaattgggatcatggtggctggatttgcatccgtgattatacaggctgtggtgatgcaaggtggaatcagcactattttaaatgatgcctatgatggtggaagattaaatttctggaattttaatcctaaccctttgcaaagacacaccttctggacaattattataggagggaccttcacatggaccagcatctacggtgtcaaccaatcccaggtgcagagatatatttcttgtaaaagcagattccaggcaaaactgtctctctacatcaatcttgtgggactctgggcaatcctcacatgctcagtgttttgtgggctcgccctatattccaggtaccatgactgtgatccttggacagccaagaaagtgtctgcaccagaccagctcatgccttatttggtactggacattctgcaagattatccaggacttcctggactttttgtggcctgtgcttacagtgggacattaagcacagtgtcctccagtattaatgccttagcagcagtaactgtggaagatctaatcaaaccttacttcagatcgctctcagaaaggtctctgtcttggatttcccaaggaatgagtgtggtgtatggagccctgtgtattggaatggctgcgctggcgtcacttatgggagctttgttgcaggcagcactcagcgtatttggtatggttggtggaccacttatgggcctgttcgctttgggcattttggttccctttgccaactcaattggagcacttgttggtctgatggctggatttgccatttctctatgggttggaattggagctcaaatatatcctccacttcctgagagaacattgccattgcaccttgatatccaaggctgtaacagcacctacaatgagacaaatttgattacaaccacagaaatgccatttactactagtgtttttcaaatatacaatgttcaaaggactccactgatggataactggtattctttatcatatctgtacttcagcactgttggaactttggtaacattattagtggggatacttgtcagtttatcaacaggtaactatctaaacattagtattaagaagcatgttttgagctataaatcacatccagtggaagatggtggaactgataatcctgctttcaaccacattgaattgaactcagatcagagtggcaagagcaatgggactcgtttgtga

SEQ ID NO: 44 - DNA, 1785 bp, Homo sapiens
atggacacgccacggggcatcggcaccttcgtggtgtgggactacgtggtgttcgcgggcatgctggtcatctcggccgccatcggcatctactacgccttcgctgggggcggccagcagacctccaaggacttcctgatgggcggccgcagaatgaccgcagtgcccgtggcgctgtccctcaccgctagcttcatgtcagccgtcactgtcctgggcaccccctccgaggtctaccgttttggggccatttttagcatctttgccttcacctacttctttgtggtggtcatcagcgcggaggtcttcctcccggtgttctacaaactgggaattaccagcacctacgaggtaaggggcagggtgggctgggaccatgcagggcgcgggggaaggggactctgcagacctctggaggcgttttcttgggggcagactgtcactgccacatcgaaatctctccccgtccatctcacaggatttgatctgtggggcgcggtagtggcaacgggggtggtctgcacattctactgcacactgggtggtcttaaagcagttatctggacagatgtttttcaaattgggatcatggtggctggatttgcatccgtgattatacaggctgtggtgatgcaaggtggaatcagcactattttaaatgatgcctatgatggtggaagattaaatttctggaattttaatcctaaccctttgcaaagacacaccttctggacaattattataggagggaccttcacatggaccagcatctacggtgtcaaccaatcccaggtgcagagatatatttcttgtaaaagcagattccaggcaaaactgtctctctacatcaatcttgtgggactctgggcaatcctcacatgctcagtgttttgtgggctcgccctatattccaggtaccatgactgtgatccttggacagccaagaaagtgtctgcaccagaccagctcatgccttatttggtactggacattctgcaagattatccaggacttcctggactttttgtggcctgtgcttacagtgggacattaagcacagtgtcctccagtattaatgccttagcagcagtaactgtggaagatctaatcaaaccttacttcagatcgctctcagaaaggtctctgtcttggatttcccaaggaatgagtgtggtgtatggagccctgtgtattggaatggctgcgctggcgtcacttatgggagctttgttgcaggcagcactcagcgtatttggtatggttggtggaccacttatgggcctgttcgctttgggcattttggttccctttgccaactcaattggagcacttgttggtctgatggctggatttgccatttctctatgggttggaattggagctcaaatatatcctccacttcctgagagaacattgccattgcaccttgatatccaaggctgtaacagcacctacaatgagacaaatttgattacaaccacagaaatgccatttactactagtgtttttcaaatatacaatgttcaaaggactccactgatggataactggtattctttatcatatctgtacttcagcactgttggaactttggtaacattattagtggggatacttgtcagtttatcaacaggatctgagcagccaagtaaggggcagtccactgtgtcaagatgccataccgttctggtgcactgtgtgtctctccattctgggtctgcctctgccttccacttcaccgtgcttctgcctcagtggtcctggtgccagtggtcttctaagtag

SEQ ID NO: 45 - DNA, 1155 bp, Homo sapiens
atggtagctgaggttgattcaatgccggctgcctcttctgtgaagaagccatttgttctcaggagcaagatgggcaagtggtgccgccactgcttcccctgctgcagggggagcggcaagagcaacgtgggcacttctggagaccaggacgactccactatgaagacactcaggagcaagatgggcaagtggtgctgccactgcttcccctgctgcagggggagtggcaagagcaacgtgggcacttctggagaccacgacgactccgctatgaagacgctcaggagcaagatgggcaagtggtgctgccactgcttcccctgctgcagggggagcggcaagagcaacgtgggtgcttggggagactacgacgacagcgccttcgtggagccgagataccacgtccgtcgagaagatctggacaagctccacagagctgcctggtggggtaaagtcgccagaaaggatctgatcgtcatgctcagggacactgacgtgaacaagcaggacaagcaaaagaggactgctctacatctggcctctgccaatgggaattcaggagtagtaaaactcctgctggacagacgatgtcaacttaatgtccttgacaacaaaaagaggacagctctgacaaaggccgtacaatgccaggaagatgaatgtgcgttaatgttgctggaacatggcactgatccaaacattccagatgagtatggaaataccactctacactacgctatctacaatgaagataaattaatggccaaagcactgctcttatacggtgctgatatcgaatcaaaaaacaagcatggcctcacaccactgctacttggtgtacatgagcaaaaacagcaagtggtgaaatttttaatcaagaaaaaagcgaatttaaatgcgctggatagatatggaagaactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 46 - DNA, 843 bp, Homo sapiens
atgagaggggtttcctgtctccaggtcctgctccttctggtgctgggagctgctgggactcagggaaggaagtctgcagcctgcgggcagccccgcatgtccagtcggatcgttgggggccgggatggccgggacggagagtggccgtggcaggcgagcatccagcatcgtggggcacacgtgtgcggggggtcgctcatcgccccccagtgggtgctgacagcggcgcactgcttccccaggagggcactgccagctgagtaccgcgtgcgcctgggggcgctgcgtctgggctccacctcgccccgcacgctctcggtgcccgtgcgacgggtgctgctgcccccggactactccgaggacggggcccgcggcgacctggcactgctgcagctgcgtcgcccggtgcccctgagcgctcgcgtccaacccgtctgcctgcccgtgcccggcgcccgcccgccgcccggcacaccatgccgggtcaccggctggggcagcctccgcccaggagtgcccctcccagagtggcgaccgctacaaggagtaagggtgccgctgctggactcgcgcacctgcgacggcctctaccacgtgggcgcggacgtgccccaggctgagcgcattgtgctgcctgggagtctgtgtgccggctacccccagggccacaaggacgcctgccagggtgattctgggggacctctgacctgcctgcagtctgggagctgggtcctggtgggcgtggtgagctggggcaagggttgtgccctgcccaaccgtccaggggtctacaccagtgtggccacatatagcccctggattcaggctcgcgtcagcttctaa

SEQ ID NO: 47 - DNA, 849 bp, Homo sapiens
atgagaggggtttcctgtctccaggtcctgctccttctggtgctgggtcctcagccaaccctcaccgcccctggtccttttctagcctgcgggcagccccgcatgtccagtcggatcgttgggggccgggatggccgggacggagagtggccgtggcaggcgagcatccagcatcgtggggcacacgtgtgcggggggtcgctcatcgccccccagtgggtgctgacagcggcgcactgcttccccaggagggcactgccagctgagtaccgcgtgcgcctgggggcgctgcgtctgggctccacctcgccccgcacgctctcggtgcccgtgcgacgggtgctgctgcccccggactactccgaggacggggcccgcggcgacctggcactgctgcagctgcgtcgcccggtgcccctgagcgctcgcgtccaacccgtctgcctgcccgtgcccggcgcccgcccgccgcccggcacaccatgccgggtcaccggctggggcagcctccgcccaggagtgcccctcccagagtggcgaccgctacaaggagtaagggtgccgctgctggactcgcgcacctgcgacggcctctaccacgtgggcgcggacgtgccccaggctgagcgcattgtgctgcctgggagtctgtgtgccggctacccccagggccacaaggacgcctgccagggtgattctgggggacctctgacctgcctgcagtctgggagctgggtcctggtgggcgtggtgagctggggcaagggttgtgccctgcccaaccgtccaggggtctacaccagtgtggccacatatagcccctggattcaggctcgcgtcagcttctaa

SEQ ID NO: 48 - DNA, 840 bp, Homo sapiens
atgagaggggtttcctgtctccaggtcctgctccttctggtgctgggagctgctgggactcagggaaggaagtctgcagcctgcgggcagccccgcatgtccagtcggatcgttgggggccgggatggccgggacggagagtggccgtggcaggcgagcatccagcatcgtggggcacacgtgtgcggggggtcgctcatcgccccccagtgggtgctgacagcggcgcactgcttccccaggagggcactgccagctgagtaccgcgtgcgcctgggggcgctgcgtctgggctccacctcgccccgcacgctctcggtgcccgtgcgacgggtgctgctgcccccggactactccgaggacggggcccgcggcgacctggcactgctgcagctgcgtcgcccggtgcccctgagcgctcgcgtccaacccgtctgcctgcccgtgcccggcgcccgcccgccgcccggcacaccatgccgggtcaccggctggggcagcctccgcccaggagtgcccctcccagagtggcgaccgctacaaggagtaagggtgccgctgctggactcgcgcacctgcgacggcctctaccacgtgggcgcggacgtgccccaggctgagcgcattgtgctgcctgggagtctgtgtgccggctacccccagggccacaaggacgcctgccaggtgtgcacccagcctccccagcctccggagtcccctccctgtgcccagcaccctccctccctgaactccaggacccaggacatcccaactcaggctcaggatcctggcctccaacctagaggcaccacgccaggggtctggaaccctgagaactga

SEQ ID NO: 49 - DNA, 1662 bp, Homo sapiens
atggtggacatgggggccctggacaacctgatcgccaacaccgcctacctgcaggcccggaagccctcggactgcgacagcaaagagctgcagcggcggcggcgtagcctggccctgcccgggctgcagggctgcgcggagctccgccagaagctgtccctgaacttccacagcctgtgtgagcagcagcccatcggtcgccgcctcttccgtgacttcctagccacagtgcccacgttccgcaaggcggcaaccttcctagaggacgtgcagaactgggagctggccgaggagggacccaccaaagacagcgcgctgcaggggctggtggccacttgtgcgagtgcccctgccccggggaacccgcaacccttcctcagccaggccgtggccaccaagtgccaagcagccaccactgaggaagagcgagtggctgcagtgacgctggccaaggctgaggccatggctttcttgcaagagcagccctttaaggatttcgtgaccagcgccttctacgacaagtttctgcagtggaaactcttcgagatgcaaccagtgtcagacaagtacttcactgagttcagagtgctggggaaaggtggttttggggaggtatgtgccgtccaggtgaaaaacactgggaagatgtatgcctgtaagaaactggacaagaagcggctgaagaagaaaggtggcgagaagatggctctcttggaaaaggaaatcttggagaaggtcagcagccctttcattgtctctctggcctatgcctttgagagcaagacccatctctgccttgtcatgagcctgatgaatgggggagacctcaagttccacatctacaacgtgggcacgcgtggcctggacatgagccgggtgatcttttactcggcccagatagcctgtgggatgctgcacctccatgaactcggcatcgtctatcgggacatgaagcctgagaatgtgcttctggatgacctcggcaactgcaggttatctgacctggggctggccgtggagatgaagggtggcaagcccatcacccagagggctggaaccaatggttacatggctcctgagatcctaatggaaaaggtaagttattcctatcctgtggactggtttgccatgggatgcagcatttatgaaatggttgctggacgaacaccattcaaagattacaaggaaaaggtcagtaaagaggatctgaagcaaagaactctgcaagacgaggtcaaattccagcatgataacttcacagaggaagcaaaagatatttgcaggctcttcttggctaagaaaccagagcaacgcttaggaagcagagaaaagtctgatgatcccaggaaacatcatttctttaaaacgatcaactttcctcgcctggaagctggcctaattgaacccccatttgtgccagacccttcagtggtttatgccaaagacatcgctgaaattgatgatttctctgaggttcggggggtggaatttgatgacaaagataagcagttcttcaaaaactttgcgacaggtgctgttcctatagcatggcaggaagaaattatagaaacgggactgtttgaggaactgaatgaccccaacagacctacgggttgtgaggagggtaattcatccaagtctggcgtgtgtttgttattgtaa

SEQ ID NO: 50 - DNA, 1329 bp, Homo sapiens
atgcagtgggccctgaaggtgggacattttgcccaaggaaccctcccagcctccatgccgcctttcctgatcaccctcttcctctttcactcttgctgcctccgagcaaatggccacctccgtgaaggaatgacattgctgaagactgagtttgcacttcacctctaccagagtgtggccgcgtgtagaaatgagacgaactttgtcatctctcctgctggtgtgtccctccccctggagatcctgcagtttggagcagaagggagcactggtcagcagctggcagatgccctggggtacactgtccatgacaaaagggtgaaagatttcttgcatgctgtttatgccacactacccacctccagccaaggcaccgagatggagctggcctgcagcctttttgtgcaagtgggaacgccactgtccccctgctttgtggagcacgtctcctggtgggctaacagcagcctggaaccagccgacctcagtgagcccaatagcaccgccatccagactagcgaaggggcctccagagagactgcaggtgggggccccagtgagggccctggtggctggccgtgggagcaagtcagtgcagcatttgctcagcttgtgcttgtgagcaccatgtccttccaaggcacttggcgaaagagattctcctccacagacacacagatcctgcctttcacctgtgcctatggcctcgtccttcaggtccccatgatgcaccaaacgaccgaggtcaactacggtcagttccaggacactgcaggccatcaggtgggggtgctggagcttccttacctgggaagtgcagtgagtctgttcctggtgctgccccgtgacaaagacacccccctgagccacatcgagccacacctcacagccagcaccatccacctctggaccaccagcctgaggagagccaggatggatgtgttcctgcccaggtttaggatccaaaatcaattcaacttaaaaagcattttaaattcttggggagtcaccgatctttttgatccactcaaagctaacttgaaaggaatttcaggccaagatggcttttatgtttctgaagcaatccacaaggccaagattgaagttttggaggaaggcaccaaggcatctggagccacagctctgttgttattgaaaaggtctcggattcctatttttaaagcagatcggccattcatctatttcctgagagaacctaacacaggtattacagtattttttgacaggattcagataatttatcagtgtctctctagcaacaagggctcatttgtccactatcccctcaaaaataagcattctttctag

SEQ ID NO: 51 - DNA, 3081 bp, Homo sapiens
atgctggtggtggaaagagtaatggttcttcccattgggttcccccttggtgtgagtgatgattccacactgcatggcccgatttttattcaagaaccaagtcctgtaatgttccctttggattctgaggagaaaaaagtgaagctcaattgtgaagttaaaggaaatccaaaacctcatatcaggtggaagttaaatggaacagatgttgacactggtatggatttccgctacagtgttgttgaagggagcttgttgatcaataaccccaataaaacccaagatgctggaacgtaccagtgcacagcgacaaactcgtttggaacaattgttagcagagaagcaaagcttcagtttgcttatcttgacaactttaaaacaagaacaagaagcactgtgtctgtccgtcgaggtcaaggaatggtgctactgtgtggcccgccaccccattctggagagctgagttatgcctggatcttcaatgaatacccttcctatcaggataatcgccgctttgtttctcaagagactgggaatctgtatattgccaaagtagaaaaatcagatgttgggaattatacctgtgtggttaccaataccgtgacaaaccacaaggtcctggggccacctacaccactaatattgagaaatgatggagtgatgggtgaatatgagcccaaaatagaagtgcagttcccagaaacagttccgactgcaaaaggagcaacggtgaagctggaatgctttgctttaggaaatccagtaccaactattatctggcgaagagctgatggaaagccaatagcaaggaaagccagaagacacaagtcaaatggaattcttgagatccctaattttcagcaggaggatgctggtttatatgaatgtgtagctgaaaattccagagggaaaaatgtagcaaggggacagctaactttctatgctcaacctaattggattcaaaaaataaatgatattcacgtggccatggaagaaaatgtcttttgggaatgtaaagcaaatggaaggcctaagcctacatacaagtggctaaaaaatggcgaacctctgctaactcgggatagaattcaaattgagcaaggaacactcaacataacaatagtgaacctctcagatgctggcatgtatcagtgtttggcagagaataaacatggagttatcttttccaacgcagagcttagtgttatagctgtaggtccagatttttcaagaacactcttgaaaagagtaactcttgtcaaagtgggaggtgaagttgtcattgagtgtaagccaaaagcgtctccaaaacctgtttacacctggaagaaaggaagggatatattaaaagaaaatgaaagaattaccatttctgaagatggaaacctcagaatcatcaacgttactaaatcagacgctgggagttatacctgtatagccactaaccattttggaactgctagcagtactggaaacttggtagtgaaagatccaacaagggtaatggtacccccttccagtatggatgtcactgttggagagagtattgttttaccgtgccaggtaacgcatgatcactcgctagacatcgtgtttacttggtcatttaatggacacctgatagactttgacagagatggggaccactttgaaagagttggagggcaggattcagctggtgatttgatgatccgaaacatccaactgaagcatgctgggaaatatgtctgcatggtccaaacaagtgtggacaggctatctgctgctgcagacctgattgtaagaggtcctccaggtcccccagaggctgtgacaatagacgaaatcacagataccactgctcagctctcctggagacccgggcctgacaaccacagccccatcaccatgtatgtcattcaagccaggactccattctccgtgggctggcaagcagtcagtacagtcccagaactcattgatgggaagacattcacagcgaccgtggtgggtttgaacccttgggttgaatatgaattccgcacagttgcagccaacgtgattgggattggggagcccagccgcccctcagagaaacggagaacagaagaagctctccccgaagtcacaccagcgaatgtcagtggtggcggaggcagcaaatctgaactggttataacctgggagacggtccctgaggaattacagaatggtcgaggctttggttatgtggtggccttccggccctacggtaaaatgatctggatgctgacagtgctggcctcagctgatgcctctagatacgtgttcaggaatgagagcgtgcaccccttctctccctttgaggttaaagtaggtgtcttcaacaacaaaggagaaggccctttcagtcccaccacggtggtgtattctgcagaagaagaacccaccaaaccaccagccagtatctttgccagaagtctttctgccacagatattgaagttttctgggcctccccactggagaagaatagaggacgaatacaaggttatgaggttaaatattggagacatgaagacaaagaagaaaatgctagaaaaatacgaacagttggaaatcagacatcaacaaaaatcacgaacttaaaaggcagtgtgctgtatcacttagctgtcaaggcatataattctgctgggacaggcccctctagtgcaacagtcaatgtgacaacccgaaagccaccaccaagtcaaccccccggaaacatcatatggaattcatcagactccaaaattatcctgaattgggatcaagtgaaggccctggataatgagtcggaagtaaaaggatacaaagtcttgtacagatggaacagacaaagcagcacatctgtcattgaaacaaataaaacatcggtggagctttctttgcctttcgatgaagattatataatagaaattaagccattcagcgacggaggagatggcagcagcagtgaacaaattcgaattccaaagatatcaaatgcctacgcgagaggatctggggcttccacttcgaatgcatgtacgctgtcagccatcagtacaataatgatttccctcacagctaggtccagttta

SEQ ID NO: 52 - protein, 148 aa, Homo sapiens
MSSPQRRKAMPWALSLLLMGFQLLVTYAWCSEEEMGGNNKIVQDPMFLATVEFALNTFNVQSKEEHAYRLLRVLSSWREDSMDRKMVFSMNLQLRQTVCRKFEDDIDNCPFQESLELNNTFSCFFTVETMPWKTYFELLNKTCSEGLS

SEQ ID NO: 53 - protein, 159 aa, Homo sapiens
MSSPQRRKAMPWALSLLLMGFQLLVTYAWCSEEEMGGNNKIVQDPMFLATVEFALNTFNVQSKEEHAYRLLRVLSSWREDSMDRKWRGKMVFSMNLQLRQTVCRKFEDDIDNCPFQESLELNNVRQGISFPQVHSCGCCMGCGVGTGAADKAIPRDKGK

SEQ ID NO: 54 - protein, 782 aa, Homo sapiens
MARARAGALLALWVLGAAAHPQCLDFRPPFRPTQPLRLCAQYSDFGCCDEGRDAELTRRFWALASRVDAAEWAACAGYARDLLCQECSPYAAHLYDAEDPFTPLRTVPGLCQDYCLDMWHKCRGLFRHLSTDQELWALEGNLARFCRYLSLDDTDYCFPYLLVNKNLNSNLGHVVADAKGCLQLCLEEVANGLRNPVAMVHARDGTHRFFVAEQVGLVWAYLPDRSRLGKPFLNISRVVLTSPWEGDERGFLGIAFHPSFQHNRRLYVYYSVGIRSSEWIRISEFRVSEDDENAVDHSSERIILEVKEPASNHNGGQLLFGDDGYLYIFTGDGGMAGDPFGTFGNAQNKSALLGKVLRIDVDRKERGLPYGIPPDNPFVGDPAAQPEVYALGVRNMWRCSFDRGDPSSGTGRGRLFCGDVGQNKFEEVDVVERGGNYGWRAREGFECYDRSLCANTSLNDLLPIFAYPHTVGKSVTGGYVYRGCEYPNLNGLYIFGDFMSGRLMSLQENPGTGQWQYSEICMGHGQTCEFPGLINNYYPYIISFGEDEAGELYFMSTGEPSATAPRGVVYKIIDASRRAPPGKCQIQPAQVKIRSRLIPFVPKEKFIPKTRSTPRPTARAPTRAPRRGRPTAAPPAPTPRPARPTQQPGSRRGGGRRRGRLNSASRAFRDGEVRLVRPAGLSSGSGRVEVFVGGRWGTVCDDSWNISGAAVVCRQLGFAYAVRAVKRAEFGQGGSLPILLDDVRCAGWERNLLECQHNGVGTHNCEHDEDAGVVCSHQNPDL

SEQ ID NO: 55 - protein, 766 aa, Homo sapiens
MARARAGALLALWVLGAAAHPQCLDFRPPFRPTQPLRLCAQYSDFGCCDEGRDAELTRRFWALASRVDAAEWAACAGYARDLLCQSVEWTDMQRDNEVLAKLTGWSAPGDGAVTAVENSPSLDYPGLGTTFTSCECSPYAAHLYDAEDPFTPLRTVPGLCQDYCLDMWHKCRGLFRHLSTDQELWALEGNLARFCRYLSLDDTDYCFPYLLVNKNLNSNLGHVVADAKGCLQLCLEEVANGLRNPVAMVHARDGTHRFFVAEQVGLVWAYLPDRSRLGKPFLNISRVVLTSPWEGDERGFLGIAFHPSFQHNRRLYVYYSVGIRSSEWIRISEFRVSEDDENAVDHSSERIILEVKEPASNHNGGQLLFGDDGYLYIFTGDGGMAGDPFGTFGNAQNKYVQLLIGLWVGLHIPGLLILFQRSALLGKVLRIDVDRKGARPALRHPARQPVRGRPRGAAEVYALGVRNMWRCSFDRGDPSSGTGRGRLFCGDVGQNKFEEVDVVERGGNYGWRAREGFECYDRSLCANTSLNDLLPIFAYPHTVGKSVTGGYVYRGCEYPNLNGLYIFGDFMSGRLMSLQENPGTGQWQYSEICMGHGQTCEFPGLINNYYPYIISFGEDEAGELYFMSTGEPSATAPRGVVYKIIDASSRAFRDGEVRLVRPAGLSSGSGRVEVFVGGRWGTVCDDSWNISGAAVVCRQLGFAYAVRAVKRAEFGQGGSLPILLDDVRCAGWERNLLECQHNGVGTHNCEHDEDAGVVCSHQNPDL

SEQ ID NO: 56 - protein, 606 aa, Homo sapiens
MLHTAISCWQPFLGLAVVLIFMGSTIGCPARCECSAQNKSVSCHRRRLIAIPEGIPIETKILDLSKNRLKSVNPEEFISYPLLEEIDLSDNIIANVEPGAFNNLFNLRSLRLKGNRLKLVPLGVFTGLSNLTKLDISENKIVILLDYMFQDLHNLKSLEVGDNDLVYISHRAFSGLLSLEQLTLEKCNLTAVPTEALSHLRSLISLHLKHLNINNMPVYAFKRLFHLKHLEIDYWPLLDMMPANSLYGLNLTSLSVTNTNLSTVPFLAFKHLVYLTHLNLSYNPISTIEAGMFSDLIRLQELHIVGAQLRTIEPHSFQGLRFLRVLNVSQNLLETLEENVFSSPRALEVLSINNNPLACDCRLLWILQRQPTLQFGGQQPMCAGPDTIRERSFKDFHSTALSFYFTCKKPKIREKKLQHLLVDEGQTVQLECSADGDPQPVISWVTPRRRFITTKSNGRATVLGDGTLEIRFAQDQDSGMYVCIASNAAGNDTFTASLTVKGFASDRFLYANRTPMYMTDSNDTISNGTNANTFSLDLKTILVSTAMGCFTFLGVVLFCFLLLFVWSRGKGKHKNSIDLEYVPRKNNGAVVEGEVAGPRRFNMKMI

SEQ ID NO: 57 - protein, 606 aa, Homo sapiens; identical to SEQ ID NO: 56

SEQ ID NO: 58 - protein, 107 aa, Homo sapiens
MPSPMEHTMETVLFTFHRFAGDKGYLMKEGLKVLMGKEFLGFLENQKDPLAADITMKDMDQCQDSTLNFQNLFSLTAGLTTVDNNYFVVPMKQKGTKQAELSNYSAL

SEQ ID NO: 59 - protein, 99 aa, Homo sapiens [sequence omitted]

SEQ ID NO: 60 - protein, 380 aa, Homo sapiens
MSLMVVSMACVGFFLLQGAWPHEGVHRKPSLLAHPGPLVKSEETVILQCWSDVMFEHFLLHREGKFNDTLRLTGELHDGVSKANFSIGRMTQDLAGTYRCYGSVPHSPYQLSAPSDPLDIVITGLCGKPSLSAQPRPMVKAGESVTLSCSSRSSYDIYHLSRDGEAHELRFPAVPKVNGTFQANFPLGPATHGGTYRCFGSFRDSPYEWSDLSDPLLVSVTGNPSSSWPSPTEPSFKTGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 61 - protein, 380 aa, Homo sapiens
MSLMVVSMACVGFFLLQGAWPHEGVHRKPSLLAHPGPLVKSEETVILQCWSDVMFEHFLLHREGKFNDTLRLTGELHDGVSKANFSIGRMTQDLAGTYRCYGSVPHSPYQLSAPSDPLDIVITGLCGKPSLSAQPRPMVKAGESVTLSCSSRSSYDIYHLSRDGEAHELRFPAVPKVNGTFQANFPLGPATHGGTYRCFGSFRDSPYEWSDLSDPLLVSVTGNPSSSWPSPTEPSFKTGIARHLHAVIRYSVAIILFTILPFFLLHRWCSKKKNAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 62 - protein, 351 aa, Homo sapiens [sequence omitted]

SEQ ID NO: 63 - protein, 362 aa, Homo sapiens [sequence omitted]

SEQ ID NO: 64 - protein, 270 aa, Homo sapiens
MCGRFLRRLLAEESRRSTPVGRLLLPVLLGFRLVLLAASGPGVYGDEQSEFVCHTQQPGCKAACFDAFHPLSPLRFWVFQVILVAVPSALYMGFTLYHVIWHWELSGKGKEEETLIQGREGNTDVPGAGSLRLLWAYVAQLGARLVLEGAALGLQYHLYGFQMPSSFACRREPCLGSITCNLSRPSEKTIFLKTMFGVSGFCLLFTFLELVLLGLGRWWRTWKHKSSSSKYFLTSESTRRHKKATDSLPVVETKEQFQEAGEKDTLSSCH

SEQ ID NO: 65 - protein, 228 aa, Homo sapiens
MCGRFLRRLLAEESRRSTPVGRLLLPVLLGFRLVLLAASGPGVYGDEQSEFVCHTQQPGCKAACFDAFHPLSPLRFWVFQVILVAVPSALYMGFTLYHLGARLVLEGAALGLQYHLYGFQMPSSFACRREPCLGSITCNLSRPSEKTIFLKTMFGVSGFCLLFTFLELVLLGLGRWWRTWKHKSSSSKYFLTSESTRRHKKATDSLPVVETKEQFQEAGEKDTLSSCH

SEQ ID NO: 66 - protein, 686 aa, Homo sapiens
MLLLPLLLPVLGAGSLNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGAALGAGVAALLAFCSCLVVFRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 67 - protein, 667 aa, Homo sapiens
MLLLPLLLPVLGAGSLNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVACECGLGGWGVQTAPVGGEVEEPSGTGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPVGDEESESSRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 68 - protein, 99 aa, Homo sapiens
MKPEWAHFSLWLLILSMLLHTPSGNIFPPQLRKQLRESGHLGEYCNYPLKKGTCNSYLTRFYYNTLTFLCEPFVFSGCGGNRNNFKQKYFCEKMCITEK

SEQ ID NO: 69 - protein, 257 aa, Homo sapiens
MGTVRPPRPSLLLVSTRESCLFLLFCLHLGAACPQPCRCPDHAGAVAVFCSLRGLQEVPEDIPANTVLLKLDANKISHLPDGAFQHLHRLRELDLSHNAIEAIGSATFAGLAGGLRLLDLSYNRIQRIPKDALGKLSAKIRLSHNPLHCECALQEALWELKLDPDSVDEIACHTSVQEEFVGKPLVQALDAGASLCSVPHRTTDVAMLVTMFGWFAMVIAYVVYYVRHNQEDARRHLEYLKSLPSAPASKDPIGPGP

SEQ ID NO: 70 - protein, 367 aa, Homo sapiens
MDDAAVLKRRGYLLGINLGEGSYAKVKSAYSERLKFNVAIKIIDRKKAPADFLEKFLPREIEILAMLNHCSIIKTYEIFETSHGKVYIVMELAVQGDLLELIKTRGALHEDEARKKFHQLSLAIKYCHDLDVVHRDLKCDNLLLDKDFNIKLSDFSFSKRCLRDDSGRMALSKTFCGSPAYAAPEVLQGIPYQPKVYDIWSLGVILYIMVCGSMPYDDSNIKKMLRIQKEHRVNFPRSKHLTGECKDLIYHMLQPDVNRRLHIDEILSHCWMQPKARGSPSVAINKEGESSRGTEPLWTPEPGSDKKSATKLEPEGEAQPQAQPETKPEGTAMQMSRQSEILGFPSKPSTMETEEGPPQQPPETRAQ

SEQ ID NO: 71 - protein, 542 aa, Homo sapiens
MINYKSLLALLFILASWIIFTVFQNSTKVWSALNLSISLHYWNNSTKSLFPKTPLISLKPLTETELRIKEIIEKLDQQIPPRPFTHVNTTTSATHSTATILNPRDTYCRGDQLHILLEVRDHLGRRKQYGGDFLRARMSSPALMAGASGKVTDFNNGTYLVSFTLFWEGQVSLSLLLIHPSEGVSALWSARNQGYDRVIFTGQFVNGTSQVHSECGLILNTNAELCQYLDNRDQEGFYCVRPQHMPCAALTHMYSKNKKVSYLSKQEKSLFERSNVGVEIMEKFNTISVSKCNKREKIEETCQVGMKPPVPGGYTLQGKWITTFCNQVQLDTIKINGCLKGKLIYLLGDSTLRQWIYYFPKVVKTLKFFDLHETGIFKKHLLLDAERHTQIQWKKHSYPFVTFQLYSLIDHDYIPREIDRLSGDKNTAIVITFGQHFRPFPIDIFIRRAIGVQKAIERLFLRSPATKVIIKTENIREMHIETERFGDFHGYIHYLIMKDIFKDLNVGIIDAWDMTIAYGTDTIHPPDHVIGNQINMFLNYIC

SEQ ID NO: 72 - protein, 553 aa, Homo sapiens
MFRWFGGIQGIFSNPGRGESISKEQELKDRKISQSLSFQLWSALNLSISVHYWNNSAKSLFPKTSLIPLKPLTETELRIKEIIEKLDQQIPPRPFTHVNTTTSATHSTATILNPRDTYCRGDQLDILLEVRDHLGQRKQYGGDFLRARMSSPALTAGASGKVMDFNNGTYLVSFTLFWEGQVSLSLLLIHPSEGASALWRARNQGYDKIIFKGKFVNGTSHVFTECGLTLNSNAELCEYLDDRDQEAFYCMKPQHMPCEALTYMTTRNREVSYLTDKENSLFHRSKVGVEMMKDRKHIDVTNCNKREKIEETCQVGMKPPVPGGYTLQGKWITTFCNQVQLDTIKINGCLKGKLIYLLGDSTLRQWIYYFPKVVKTLKFFDLHETGIFKKHLLLDAERHTQIQWKKHSYPFVTFQLYSLIDHDYIPREIDRLSGDKNTAIVITFGQHFRPFPIDIFIRRAIGVQKAIERLFLRSPATKVIIKTENIREMHIETERFGDFHGYIHYLIMKDIFKDLNVGIIDAWDMTIAYGTDTIHPPDHVIGNQINMFLNYIC

SEQ ID NO: 73 - protein, 370 aa, Homo sapiens
MEGVDLLGFLIITLNCNVTMVGKLWFVLTMLLRMLVIVLAGRPVYQDEQERFVCNTLQPGCANVCYDVFSPVSHLRFWLIQGVCVLLPSAVFSVYVLHRGATLAALGPRRCPDPREPASGQRRCPRPFGERGGLQVPDFSAGYIIHLLLRTLLEAAFGALHYFLFGFLAPKKFPCTRPPCTGVVDCYVSRPTEKSLLMLFLWAVSALSFLLGLADLVCSLRRRMRRRPGPPTSPSIRKQSGASGHAEGRRTDEEGGREEEGAPAPPGARAGGEGAGSPRRTSRVSGHTKIPDEDESEVTSSASEKLGRQPRGRPHREAAQDPRGSGSEEQPSAAPSRLAAPPSCSSLQPPDPPASSSGAPHLRARKSEWV

SEQ ID NO: 74 - protein, 356 aa, Homo sapiens
MPASSLPGKLWFVLTMLLRMLVIVLAGRPVYQDEQERFVCNTLQPGCANVCYDVFSPVSHLRFWLIQGVCVLLPSAVFSVYVLHRGATLAALGPRRCPDPREPASGQRRCPRPFGERGGLQVPDFSAGYIIHLLLRTLLEAAFGALHYFLFGFLAPKKFPCTRPPCTGVVDCYVSRPTEKSLLMLFLWAVSALSFLLGLADLVCSLRRRMRRRPGPPTSPSIRKQSGASGHAEGRRTDEEGGREEEGAPAPPGARAGGEGAGSPRRTSRVSGHTKIPDEDESEVTSSASEKLGRQPRGRPHREAAQDPRGSGSEEQPSAAPSRLAAPPSCSSLQPPDPPASSSGAPHLRARKSEWV

SEQ ID NO: 75 - protein, 861 aa, Homo sapiens
MAMRSGRHPSLLLLLVLLLWLLQVSSPFVETGSGFIIQAGVQWHDQGSRARHHARLVFVFLLETGFHHVGQAGLKLLTSSDLPASASQSVRMTGLRHCALPSFIMICHYKFQEKIYQPLRRSKRRWVITTLELEEEDPGPFPKLIGELFNNMSYNMSLMYLISGPGVDEYPEIGLFSLEDHENGRIYVHRPVDREMTPSFTVYFDVVERSTGKIVDTSLIFNIRISDVNDHAPQFPEKEFNITVQENQSAGQPIFQMLAVDLDEENTPNSQVLYFLISQTPLLKESGFRVDRLSGEIRLSGCLDYETAPQFTLLIRARDCGEPSLSSTTTVHVDVQEGNNHRPAFTQENYKVQIPEGRASQGVLRLLVQDRDSPFTSAWRAKFNILHGNEEGHFDISTDPETNEGILNVIKPLDYETRPAQSLIIVVENEERLVFCERGKLQPPRKAAASATVSVQVTDANDPPAFHPQSFIVNKEEGARPGTLLGTFNAMDPDSQIRYELVHDPANWVSVDKNSGVVITVEPIDRESPHVNNSFYVIIIHAVDDGFPPQTATGTLMLFLSDINDNVPTLRPRSRYMEVCESAVHEPLHIEAEDPDLEPFSDPFTFELDNTWGNAEDTWKLGRNWGQSVELLTLRSLPRGNYLVPLFIGDKQGLSQKQTVHVRICPCASGLTCVELADAEVGLHVGALFPVCAAFVALAVALLFLLRCYFVLEPKRHGCSVSNDEGHQTLVMYNAESKGTSAQTWSDVEGQRPALLICTAAAGPTQGVKAYPDATMHRQLLAPVEGRMAETLNQKLHVANVLEDDPGYLPHVYSEEGECGGAPSLSSLASLEQELQPDLLDSLGSKATPFEEIYSESGVPS

SEQ ID NO: 76 - protein, 116 aa, Homo sapiens
MRECRPGVSTGPLHEVRCHYKFQEKIYQPLRRSKRRWVITTLELEEEDPGPFPKLIGELFNNMSYNMSLMYLISGPGVDEYPEIGLFSLEDHENGRIYVHRPVDREMTPSFTVSKT

SEQ ID NO: 77 - protein, 922 aa, Homo sapiens
MAMRSGRHPSLLLLLVLLLWLLQVSSPFVETGSGFIIQAGVQWHDQGSRARHHARLVFVFLLETGFHHVGQAGLKLLTSSDLPASASQSVRMTGLRHCALPSFIMICHYKFQEKIYQPLRRSKRRWVITTLELEEEDPGPFPKLIGELFNNMSYNMSLMYLISGPGVDEYPEIGLFSLEDHENGRIYVHRPVDREMTPSFTVYFDVVERSTGKIVDTSLIFNIRISDVNDHAPQFPEKEFNITVQENQSAGQPIFQMLAVDLDEENTPNSQVLYFLISQTPLLKESGFRVDRLSGEIRLSGCLDYETAPQFTLLIRARDCGEPSLSSTTTVHVDVQEGNNHRPAFTQENYKVQIPEGRASQGVLRLLVQDRDSPFTSAWRAKFNILHGNEEGHFDISTDPETNEGILNVIKPLDYETRPAQSLIIVVENEERLVFCERGKLQPPRKAAASATVSVQVTDANDPPAFHPQSFIVNKEEGARPGTLLGTFNAMDPDSQIRYELVHDPANWVSVDKNSGVVITVEPIDRESPHVNNSFYVIIIHAVDDGFPPQTATGTLMLFLSDINDNVPTLRPRSRYMEVCESAVHEPLHIEAEDPDLEPFSDPFTFELDNTWGNAEDTWKLGRNWGQSVELLTLRSLPRGNYLVPLFIGDKQGLSQKQTVHVRICPCASGLTCVELADAEVGLHVGALFPVCAAFVALAVALLFLLRCYFVLEPKRHGCSVSNDEGHQTLVMYNAESKGTSAQTWSDVEGQRPALLICTAAAGPTQGVKAYPDATMHRQLLAPVEGRMAETLNQSKERNRFSLSRGCIIPQGRATAGRGLPQDIYKEMMPRRLTQTGKRKHGALARTPSFKKVVYDHKEDEENKAGRKQRSHLFKVMQLRNEQGGVRVQSAHSPSPLNKKACFPGDYRGESAGGHNCRAVSG

SEQ ID NO: 78 - protein, 5635 aa, Homo sapiens
MISWEVVHTVFLFALLYSSLAQDASPQSEIRAEEIPEGASTLAFVFDVTGSMYDDLVQVIEGASKILETSLKRPKRPLFNFALVPFHDPEIGPVTITTDPKKFQYELRELYVQGGGDCPEMSIGAIKIALEISLPGSFIYVFTDARSKDYRLTHEVLQLIQQKQSQVVFVLTGDCDDRTHIGYKVYEEIASTSSGQVFHLDKKQVNEVLKWVEEAVQASKVHLLSTDHLEQAVNTWRIPFDPSLKEVTVSLSGPSPMIEIRNPLGKLIKKGFGLHELLNIHNSAKVVNVKEPEAGMWTVKTSSSGRHSVRITGLSTIDFRAGFSRKPTLDFKKTVSRPVQGIPTYVLLNTSGISTPARIDLLELLSISGSSLKTIPVKYYPHRKPYGIWNISDFVPPNEAFFLKVTGYDKDDYLFQRVSSVSFSSIVPDAPKVTMPEKTPGYYLQPGQIPCSVDSLLPFTLSFVRNGVTLGVDQYLKESASVNLDIAKVTLSDEGFYECIAVSSAGTGRAQTFFDVSEPPPVIQVPNNVTVTPGERAVLTCLIISAVDYNLTWQRNDRDVRLAEPARIRTLANLSLELKSVKFNDAGEYHCMVSSEGGSSAASVFLTVQEPPKVTVMPKNQSFTGGSEVSIMCSATGYPKPKIAWTVNDMFIVGSHRYRMTSDGTLFIKNAAPKDAGIYGCLASNSAGTDKQNSTLRYIEAPKLMVVQSELLVALGDITVMECKTSGIPPPQVKWFKGDLELRPSTFLIIDPLLGLLKIQETQDLDAGDYTCVAINEAGRATGKITLDVGSPPVFIQEPADVSMEIGSNVTLPCYVQGYPEPTIKWRRLDNMPIFSRPFSVSSISQLRTGALFILNLWASDKGTYICEAENQFGKIQSETTVTVTGLVAPLIGISPSVANVIEGQQLTLPCTLLAGNPIPERRWIKNSAMLLQNPYITVRSDGSLHIERVQLQDGGEYTCVASNVAGTNNKTTSVVVHVLPTIQHGQQILSTIEGIPVTLPCKASGNPKPSVIWSKKGELISTSSAKFSAGADGSLYVVSPGGEESGEYVCTATNTAGYAKRKVQLTVYVRPRVFGDQRGLSQDKPVEISVLAGEEVTLPCEVKSLPPPIITWAKETQLISPFSPRHTFLPSGSMKITETRTSDSGMYLCVATNIAGNVTQAVKLNVHVPPKIQRGPKHLKVQVGQRVDIPCNAQGTPLPVITWSKGGSTMLVDGEHHVSNPDGTLSIDQATPSDAGIYTCVATNIAGTDETEITLHVQEPPTVEDLEPPYNTTFQERVANQRIEFPCPAKGTPKPTIKWLHNGRELTGREPGISILEDGTLLVIASVTPYDNGEYICVAVNEAGTTERKYNLKVHVPPVIKDKEQVTNVSVLLNQLTNLFCEVEGTPSPIIMWYKDNVQVTESSTIQTVNNGKILKLFRATPEDAGRYSCKAINIAGTSQKYFNIDVLVPPTIIGTNFPNEVSVVLNRDVALECQVKGTPFPDIHWFKDGKPLFLGDPNVELLDRGQVLHLKNARRNDKGRYQCTVSNAAGKQAKDIKLTIYNPPSIKGGNVTTDISVLINSLIKLECETRGLPMPAITWYKDGQPIMSSSQALYIDKGQYLHIPRAQVSDSATYTCHVANVAGTAEKSFHVDVYVPPMIEGNLATPLNKQVVIAHSLTLECKAAGNPSPILTWLKDGVPVKANDNIRIEAGGKKLEIMSAQEIDRGQYICVATSVAGEKEIKYEVDVLVPPAIEGGDETSYFIVMVNNLLELDCHVTGSPPPTIMWLKDGQLIDERDGFKILLNGRKLVIAQAQVSNTGLYRCMAANTAGDHKKEFEVTVHVPPTIKSSGLSERVVVKYKPVALQCIANGIPNPSITWLKDDQPVNTAQGNLKIQSSGRVLQIAKTLLEDAGRYTCVATNAAGETQQHIQLHVHEPPSLEDAGKMLNETVLVSNPVQLECKAAGNPVPVITWYKDNRLLSGSTSMTFLNRGQIIDIESAQISDAGIYKCVAINSAGATELFYSLQVHVAPSISGSNNMVAVVVNNPVRLECEARGIPAPSLTWLKDGSPVSSFSNGLQVLSGGRILALTSAQISDTGRYTCVAVNAAGEKQRDIDLRVYVPPNIMGEEQNVSVLISQAVELLCQSDAIPPPTLTWLKDGHPLLKKPGLSISENRSVLKIEDAQVQDTGRYTCEATNVAGKTEKNYNVNIWVPPNIGGSDELTQLTVIEGNLISLLCESSGIPPPNLIWKKKGSPVLTDSMGRVRILSGGRQLQISIAEKSDAALYSCVASNVAGTAKKEYNLQVYIRPTITNSGSHPTEIIVTRGKSISLECEVQGIPPPTVTWMKDGHPLIKAKGVEILDEGHILQLKNIHVSDTGRYVCVAVNVAGMTDKKYDLSVHAPPSIIGNHRSPENISVVEKNSVSLTCEASGIPLPSITWFKDGWPVSLSNSVRILSGGRMLRLMQTTMEDAGQYTCVVRNAAGEERKIFGLSVLVPPHIVGENTLEDVKVKEKQSVTLTCEVTGNPVPEITWHKDGQPLQEDEAHHIISGGRFLQITNVQVPHTGRYTCLASSPAGHKSRSFSLNVFVSPTIAGVGSDGNPEDVTVILNSPTSLVCEAYSYPPATITWFKDGTPLESNRNIRILPGGRTLQILNAQEDNAGRYSCVATNEAGEMIKHYEVKVYIPPIINKGDLWGPGLSPKEVKIKVNNTLTLECEAYAIPSASLSWYKDGQPLKSDDHVNIAANGHTLQIKEAQISDTGRYTCVASNIAGEDELDFDVNIQVPPSFQKLWEIGNMLDTGRNGEAKDVIINNPISLYCETNAAPPPTLTWYKDGHPLTSSDKVLILPGGRVLQIPRAKVEDAGRYTCVAVNEAGEDSLQYDVRVLVPPIIKGANSDLPEEVTVLVNKSALIECLSSGSPAPRNSWQKDGQPLLEDDHHKFLSNGRILQILNTQITDIGRYVCVAENTAGSAKKYFNLNVHVPPSVIGPKSENLTVVVNNFISLTCEVSGFPPPDLSWLKNEQPIKLNTNTLIVPGGRTLQIIRAKVSDGGEYTCIAINQAGESKKKFSLTVYVPPSIKDHDSESLSVVNVREGTSVSLECESNAVPPPVITWYKNGRMITESTHVEILADGQMLHIKKAEVSDTGQYVCRAINVAGRDDKNFHLNVYVPPSIEGPEREVIVETISNPVTLTCDATGIPPPTIAWLKNHKRIENSDSLEVRILSGGSKLQIARSQHSDSGNYTCIASNMEGKAQKYYFLSIQVPPSVAGAEIPSDVSVLLGENVELVCNANGIPTPLIQWLKDGKPIASGETERIRVSANGSTLNIYGALTSDTGKYTCVATNPAGEEDRIFNLNVYVTPTIRGNKDEAEKLMTLVDTSINIECRATGTPPPQINWLKNGLPLPLSSHIRLLAAGQVIRIVRAQVSDVAVYTCVASNRAGVDNKHYNLQVFAPPNMDNSMGTEEITVLKGSSTSMACITDGTPAPSMAWLRDGQPLGLDAHLTVSTHGMVLQLLKAETEDSGKYTCIASNEAGEVSKHFILKVLEPPHINGSEEHEEISVIVNNPLELTCIASGIPAPKMTWMKDGRPLPQTDQVQTLGGGEVLRISTAQVEDTGRYTCLASSPAGDDDKEYLVRVHVPPNIAGTDEPRDITVLRNRQVTLECKSDAVPPPVITWLRNGERLQATPRVRILSGGRYLQINNADLGDTANYTCVASNIAGKTTREFILTVNVPPNIKGGPQSLVILLNKSTVLECIAEGVPTPRITWRKDGAVLAGNHARYSILENGFLHIQSAHVTDTGRYLCMATNAAGTDRRRIDLQVHVPPSIAPGPTNMTVIVNVQTTLACEATGIPKPSINWRKNGHLLNVDQNQNSYRLLSSGSLVIISPSVDDTATYECTVTNGAGDDKRTVDLTVQVPPSIADEPTDFLVTKHAPAVITCTASGVPFPSIHWTKNGIRLLPRGDGYRILSSGAIEILATQLNHAGRYTCVARNAAGSAHRHVTLHVHEPPVIQPQPSELHVILNNPILLPCEATGTPSPFITWQKEGINVNTSGRNHAVLPSGGLQISRAVREDAGTYMCVAQNPAGTALGKIKLNVQVPPVISPHLKEYVIAVDKPITLSCEADGLPPPDITWHKDGRAIVESIRQRVLSSGSLQIAFVQPGDAGHYTCMAANVAGSSSTSTKLTVHVPPRIRSTEGHYTVNENSQAILPCVADGIPTPAINWKKDNVLLANLLGKYTAEPYGELILENVVLEDSGFYTCVANNAAGEDTHTVSLTVHVLPTFTELPGDVSLNKGEQLRLSCKATGIPLPKLTWTFNNNIIPAHFDSVNGHSELVIERVSKEDSGTYVCTAENSVGFVKAIGFVYVKEPPVFKGDYPSNWIEPLGGNAILNCEVKGDPTPTIQWNRKGVDIEISHRIRQLGNGSLAIYGTVNEDAGDYTCVATNEAGVVERSMSLTLQSPPIITLEPVETVINAGGKIILNCQATGEPQPTITWSRQGHSISWDDRVNVLSNNSLYIADAQKEDTSEFECVARNLMGSVLVRVPVIVQVHGGFSQWSAWRACSVTCGKGIQKRSRLCNQPLPANGGKPCQGSDLEMRNCQNKPCPVDGSWSEWSLWEECTRSCGRGNQTRTRTCNNPSVQHGGRPCEGNAVEIIMCNIRPCPVHGAWSAWQPWGTCSESCGKGTQTRARLCNNPPPAFGGSYCDGAETQMQVCNERNCPIHGKWATWASWSACSVSCGGGARQRTRGCSDPVPQYGGRKCEGSDVQSDFCNSDPCPTHGNWSPWSGWGTCSRTCNGGQMRRYRTCDNPPPSNGGRACGGPDSQIQRCNTDMCPVDGSWGSWHSWSQCSASCGGGEKTRKRLCDHPVPVKGGRPCPGDTTQVTRCNVQACPGGPQRARGSVIGNINDVEFGIAFLNATITDSPNSDTRIIRAKITNVPRSLGSAMRKIVSILNPIYWTTAKEIGEAVNGFTLTNAVFKRETQVEFATGEILQMSHIARGLDSDGSLLLDIVVSGYVLQLQSPAEVTVKDYTEDYIQTGPGQLYAYSTRLFTIDGISIPYTWNHTVFYDQAQGRMPFLVETLHASSVESDYNQIEETLGFKIHASISKGDRSNQCPSGFTLDSVGPFCADEDECAAGNPCSHSCHNAMGTYYCSCPKGLTIAADGRTCQDIDECALGRHTCHAGQDCDNTIGSYRCVVRCGSGFRRTSDGLSCQDINECQESSPCHQRCFNAIGSFHCGCEPGYQLKGRKCMDVNECRQNVCRPDQHCKNTRGGYKCIDLCPNGMTKAENGTCIDIDECKDGTHQCRYNQICENTRGSYRCVCPRGYRSQGVGRPCMDINECEQVPKPCAHQCSNTPGSFKCICPPGQHLLGDGKSCAGLERLPNYGTQYSSYNLARFSPVRNNYQPQQHYRQYSHLYSSYSEYRNSRTSLSRTRRTIRKTCPEGSEASHDTCVDIDECENTDACQHECKNTFGSYQCICPPGYQLTHNGKTCQDIDECLEQNVHCGPNRMCFNMRGSYQCIDTPCPPNYQRDPVSGFCLKNCPPNDLECALSPYALEYKLVSLPFGIATNQDLIRLVAYTQDGVMHPRTTFLMVDEEQTVPFALRDENLKGVVYTTRPLREAETYRMRVRASSYSANGTIEYQTTFIVYIAVSAYPY

SEQ ID NO: 79 - protein, 962 aa, Homo sapiens
MEHGALGSLGEHAAKVVGKVLRQEQDFVITHHQRLVGPTVMEQKHRCKFAMKEIVQFMASGRLGPVGVPVLCHVEEVPDREQGAAPTLCPSMEEGNAKGVMSRVIFATVTLAQVSVGNTHGNWSPWSGWGTCSRTCNGGQMRRYRTCDNPPPSNGGRACGGPDSQIQRCNTDMCPVDGSWGSWHSWSQCSASCGGGEKTRKRLCDHPVPVKGGRPCPGDTTQVTRCNVQACPGGPQRARGSVIGNINDVEFGIAFLNATITDSPNSDTRIIRAKITNVPRSLGSAMRKIVSILNPIYWTTAKEIGEAVNGFTLTNAVFKRETQVEFATGEILQMSHIARGLDSDGSLLLDIVVSGYVLQLQSPAEVTVKDYTEDYIQTGPGQLYAYSTRLFTIDGISIPYTWNHTVFYDQAQGRMPFLVETLHASSVESDYNQIEETLGFKIHASISKGDRSNQCPSGFTLDSVGPFCADEDECAAGNPCSHSCHNAMGTYYCSCPKGLTIAADGRTCQDIDECALGRHTCHAGQDCDNTIGSYRCVVRCGSGFRRTSDGLSCQDINECQESSPCHQRCFNAIGSFHCGCEPGYQLKGRKCMDVNECRQNVCRPDQHCKNTRGGYKCIDLCPNGMTKAENGTCIDIDECKDGTHQCRYNQICENTRGSYRCVCPRGYRSQGVGRPCMDINECEQVPKPCAHQCSNTPGSFKCICPPGQHLLGDGKSCAGLERLPNYGTQYSSYNLARTRRTIRKTCPEGSEASHDTCVDIDECENTDACQHECKNTFGSYQCICPPGYQLTHNGKTCQDIDECLEQNVHCGPNRMCFNMRGSYQCIDTPCPPNYQRDPVSGFCLKNCPPNDLECALSPYALEYKLVSLPFGIATNQDLIRLVAYTQDGVMHPRTTFLMVDEEQTVPFALRDENLKGVVYTTRPLREAETYRMRVRASSYSANGTIEYQTTFIVYIAVSAYPY

SEQ ID NO: 80 - protein, 1031 aa, Homo sapiens
MLFYTGLKCGIRMVNMKSKEPAVGSRFFSRISSWRNSTVTGHPWQVSLKSDEHHFCGGSLIQEDRVVTAAHCLDSLSEKQLKNITVTSGEYSLFQKDKQEQNIPVSKIITHPEYNSREYMSPDIALLYLKHKVKFGNAVQPICLPDSDDKVEPGILCLSSGWGKISKTSEYSNVLQEMELPIMDDRACNTVLKSMNLPPLGRTMLCAGFPDWGMDACQGDSGGPLVCRRGGGIWILAGITSWVAGCAGGSVPVRNNHVKASLGIFSKVSELMDFITQNLFTDCKPQGTVLFGESGKICYPHSKGDYYSHNCLYVWKIMVPEDKIILIKFTSLDMEKQVGCDHDYVSLRSSSGVLFSKVCGKILPSPLLAETSEAMVPFVSDTEDSGSGFELTVTAVQKSEAGSGCGSLAILVEEGTNHSAKYPDLYPSNTRCHWFICAPEKHIIKLTFEDFAVKFSPNCIYDAVVIYGDSEEKHKLAKLCGMLTITSIFSSSNMTVIYFKSDGKNRLQGFKARFTILPSESLNKFEPKLPPQNNPVSTVKAILHDVCGIPPFSPQWLSRRIAGGEEACPHCWPWQVGLRFLGDYQCGGAIINPVWILTAAHCVQLKNNPLSWTIIAGDHDRNLKESTEQVRRAKHIIVHEDFNTLSYDSDIALIQLSSPLEYNSVVRPVCLPHSAEPLFSSEICAVTGWGSISADGGLASRLQQIQVHVLEREVCEHTYYSAHPGGITEKMICAGFAASGEKDFCQGDSGGPLVCRHENGPFVLYGIVSWGAGCVQPWKPGVFARVMIFLDWIQSKINGKLFSNVIKTITSFFRVGLGTVSCCSEAELEKPRGFFPTPRYLLDYRGRLECSWVLRVSASSMAKFTIEYLSLLGSPVCQDSVLIIYEERHSKRKTAGGLHGRRLYSMTFMSPGPLVRVTFHALVRGAFGISYIVLKVLGPKDSKITRLSQSSNREHLVPCEDVLLTKPEGIMQIPRNSHRTTMGCQWRLVAPLNHIIQLNIINFPMKPTTFVCHGHLRVYEGFGPGKKLIGN

SEQ ID NO: 81 - protein, 44 aa, Homo sapiens
MSDKPDMAEIEKFDKSKLKKTEMQEKNPLPSKETIEQEKQASES

SEQ ID NO: 82 - protein, 440 aa, Homo sapiens
MAENNENISKNVDVRPKTSRSRSADRKDGYVWSGKKLSWSKKSESYSDAETVNGIEKTEVSLRNQERKHSCSSIELDLDHSCGHRFLGRSLKQKLQDAVGQCFPIKNCSSRHSSGLPSKRKIHISELMLDKCPFPPRSDLAFRWHFIKRHTAPINSKSDEWVSTDLSQTELRDGQLKRRNMEENINCFSHTNVQPCVITTDNALCREGPMTGSVMNLVSNNSIEDSDMDSDDEILTLCTSSRKRNKPKWDLDDEILQLETPPKYHTQIDYVHCLVPDLLQINNNPCYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPLIRTFPFSLQHICRTVICNCTTYDGIDALPIPSSMKLYLKEYHYKSKVRVLRIDAPEQQC

SEQ ID NO: 83 - protein, 592 aa, Homo sapiens
MTCWLCVLSLPLLLLPAAPPPAGGCPARCECTVQTRAVACTRRRLTAVPDGIPAETRLLELSRNRIRCLNPGDLAALPALEELDLSENAIAHVEPGAFANLPRLRVLRLRGNQLKLIPPGVFTRLDNLTLLDLSENKLVILLDYTFQDLHSLRRLEVGDNDLVFVSRRAFAGLLALEELTLERCNLTALSGESLGHLRSLGALRLRHLAIASLEDQNFRRLPGLLHLEIDNWPLLEEVAAGSLRGLNLTSLSVTHTNITAVPAAALRHQAHLTCLNLSHNPISTVPRGSFRDLVRLRELHLAGALLAVVEPQAFLGLRQIRLLNLSNNLLSTLEESTFHSVNTLETLRVDGNPLACDCRLLWIVQRRKTLNFDGRLPACATPAEVRGDALRNLPDSVLFEYFVCRKPKIRERRLQRVTATAGEDVRFLCRAEGEPAPTVAWVTPQHRPVTATSAGRARVLPGGTLEIQDARPQDSGTYTCVASNAGGNDTYFATLTVRPEPAANRTPGEAHNETLAALRAPLDLTTILVSTAMGCITFLGVVLFCFVLLFVWSRGRGQHKNNFSVEYSFRKVDGPAAAAGQGGARKFNMKMI

SEQ ID NO: 84 - protein, 569 aa, Homo sapiens
MKKKLVVLGLLAVVLVLVIVSLCLWLPSASKEPDNHVYTRAAVAADAKQCLEIGRDTLRDGGSAVDAAIAALLCVGLMNAHSMGIGVGLFLTIYNSTTRKAEVINAREVAPRLAFASMFNSSEQSQKGGLSVAVPGEIRGYELAHQRHGRLPWARLFQPSIQLARQGFPVGKGLAAVLENKRTVIEQQPVLCEVFCRDRKVLWEGERLTLPRLADTYEMLAIEGAQAFYNGSLMAQIVKDIQAAGGIVTAEDLNNYCAELIEHPLNISLGDAVLYMPSARLSGPVLALILNILKGYNFSRESVETPEQKGLTYYRIVEAFRFAYAKRTLLGDPKFVDVTEVVRNMTSEFFTAQLRSQISDHTTHPISYYKPEFYTPDDGGTAHLSVVAEDGSAVSATSTINLYFGSKVCSPVSGILFNNEWTTSALPAFTNEFGAPPSPANFIQPGKQPLLSMCPTIMVGQDGQVRMVVGAAGGTQITTDTALAIIYNLWFGYDVKRAVEEPRLHNKLLPNVTTVERNIDQAVTAALETRHHHTQIASTFIAVVQAIVRTAGGWAAASDSRKGGEPAGY

SEQ ID NO: 85 - protein, 155 aa, Homo sapiens
MFEPQWVDRDQAGPGKVGCCLTWLGGSFGSKVCSPVSGILFNNEWTTSALPAFTNEFGAPPSPANFIQPGKQPLLSMCPTIMVGQDGQVRMVVGAAGGTQITTDTALAVTAALETRHHHTQIASTFIAVVQAIVRTAGGWAAASDSRKGGEPAGY

SEQ ID NO: 86 - protein, 384 aa, Homo sapiens
MVAEAGSMPAASSVKKPFGLRSKMGKWCRHCFPWCRGSGKSNVGTSGDHDDSAMKTLRSKMGKWCRHCFPWCRGSSKSNVGTSGDHDDSAMKTLRSKMGKWCCHCFPCCRGSGKSKVGPWGDYDDSAFMEPRYHVRREDLDKLHRAAWWGKVPRKDLIVMLKDTDMNKKDKQKRTALHLASANGNSEVVKLLLDRRCQLNILDNKKRTALTKAVQCREDECALMLLEHGTDPNIPDEYGNTALHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHNVICQLLSDYKEKQMLKVSSENSNPGNVSRTRNK

SEQ ID NO: 87 - protein, 1021 aa, Homo sapiens
MALRGAAGATDTPVSSAGGAPGGSASSSSTSSGGSASAGAGLWAALYDYEARGEDELSLRRGQLVEVLSQDAAVSGDEGWWAGQVQRRLGIFPANYVAPCRPAASPAPPPSRPSSPVHVAFERLELKELIGAGGFGQVYRATWQGQEVAVKAARQDPEQDAAAAAESVRREARLFAMLRHPNIIELRGVCLQQPHLCLVLEFARGGALNRALAARRIPPHVLVNWAVQIARGMLYLHEEAFVPILHRDLKSSNILLLEKIEHDDICNKTLKITDFGLAREWHRTTKMSTAGTYAWMAPEVIKSSLFSKGSDIWSYGVLLWELLTGEVPYRGIDGLAVAYGVAVNKLTLPIPSTCPEPFAKLMKECWQQDPHIRPSFALILEQLTAIEGAVMTEMPQESFHSMQDDWKLEIQQMFDELRTKEKELRSREEELTRAALQQKSQEELLKRREQQLAEREIDVLERELNILIFQLNQEKPKVKKRKGKFKRSRLKLKDGHRISLPSDFQHKITVQASPNLDKRRSLNSSSSSPPSSPTMMPRLRAIQLTSDESNKTWGRNTVFRQEEFEDVKRNFKKKGCTWGPNSIQMKDRTDCKERIRPLSDGNSPWSTILIKNQKTMPLASLFVDQPGSCEEPKLSPDGLEHRKPKQIKLPSQAYIDLPLGKDAQRENPAEAESWEEAASANAATVSIEMTPTNSLSRSPQRKKTESALYGCTVLLASVALGLDLRELHKAQAAEEPLPKEEKKKREGIFQRASKSRRSASPPTSLPSTCGEASSPPSLPLSSALGILSTPSFSTKCLLQMDSEDPLVDSAPVTCDSEMLTPDFCPTAPGSGREPALMPRLDTDCSVSRNLPSSFLQQTCGNVPYCASSKHRPSHHRRTMSDGNPTPTGATIISATGASALPLCPSPAPHSHLPREVSPKKHSTVHIVPQRRPASLRSRSDLPQAYPQTAVSQLAQTACVVGRPGPHPTQFLAAKERTKSHVPSLLDADVEGQSRDYTVPLCRMRSKTSRPSIYELEKEFLS

SEQ ID NO: 88 - protein, 384 aa, Homo sapiens
MVAEVCSMPAASAVKKPFDLRSKMGKWCHHRFPCCRGSGKSNMGTSGDHDDSFMKTLRSKMGKCCHHCFPCCRGSGTSNVGTSGDHDNSFMKTLRSKMGKWCCHCFPCCRGSGKSNVGTWGDYDDSAFMEPRYHVRREDLDKLHRAAWWGKVPRKDLIVMLRDTDMNKRDKQKRTALHLASANGNSEVVQLLLDRRCQLNVLDNKKRTALIKAVQCQEDECVLMLLEHGADGNIQDEYGNTALHYAIYNEDKLMAKALLLYGADIESKNKCGLTPLLLGVHEQKQQVVKFLIKKKANLNALDRYGRTALILAVCCGSASIVNLLLEQNVDVSSQDLSGQTAREYAVSSHHHVICELLSDYKEKQMLKISSENSNPENVSRTRNK

SEQ ID NO: 89 - protein, 336 aa, Homo sapiens
MWATCCNWFCLDGQPEEVPPPQGARMQAYSNPGYSSFPSPTGLEPSCKSCGAHFANTARKQQTCLDCKKNFCMTCSSQVGNGPRLCLLCQRFRATAFQREELMKMKVKDLRDYLSLHDISTEMCREKEELVLLVLGQQPVISQEDRTRASTLSPDFPEQQAFLTQPHSSMVPPTSPNLPSSSAQATSVPPAQVQENQQSIDSEDSFVPGRRASLSDLTDLEDIEGLTVRQLKEILARNFVNYKGCCEKWELMERVTRLYKDQKGLQHLVSGAEDQNGGAVPSGLEENLCKICMDSPIDCVLLECGHMVTCTKCGKRMNECPICRQYVIRAVHVFRS

SEQ ID NO: 90 - protein, 356 aa, Homo sapiens
MWATCCNWFCLDGQPEEVPPPQGARMQAYSNPGYSSFPSPTGLEPSCKSCGAHFANTARKQQTCLDCKKNFCMTCSSQVGNGPRLCLLCQRFRATAFQREELMKMKVKDLRDYLSLHDISTEMCREKEELVLLVLGQQPVISQEDRTRASTLSPDFPEQQAFLTQPHSSMVPPTSPNLPSSSAQATSVPPAQVQENQQANGHVSQDQEEPVYLESVARVPAEDETQSIDSEDSFVPGRRASLSDLTDLEDIEGLTVRQLKEILARNFVNYKGCCEKWELMERVTRLYKDQKGLQHLGGAVPSGLEENLCKICMDSPIDCVLLECGHMVTCTKCGKRMNECPICRQYVIRAVHVFRS

SEQ ID NO: 91 - protein, 575 aa, Homo sapiens
MSGNWVHPGQILIWAIWVLAAPTKGPSAEGPQRNTRLGWIQGKQVTVLGSPVPVNVFLGVPFAAPPLGSLRFTNPQPASPWDNLREATSYPNLCLQNSEWLLLDQHMLKVHYPKFGVSEDCLYLNIYAPAHADTGSKLPVLVWFPGGAFKTGSASIFDGSALAAYEDVLVVVVQYRLGIFGFFTTWDQHAPGNWAFKDQVAALSWVQKNIEFFGGDPSSVTIFGESAGAISVSSLILSPMAKGLFHKAIMESGVAIIPYLEAHDYEKSEDLQVVAHFCGNNASDSEALLRCLRTKPSKELLTLSQKTKSFTRVVDGAFFPNEPLDLLSQKAFKAIPSIIGVNNHECGFLLPMKEAPEILSGSNKSLALHLIQNILHIPPQYLHLVANEYFHDKHSLTEIRDSLLDLLGDVFFVVPALITARYHRDAGAPVYFYEFRHRPQCFEDTKPAFVKADHADEVRFVFGGAFLKGDIVMFEGATEEEKLLSRKMMKYWATFARTGNPNGNDLSLWPAYNLTEQYLQLDLNMSLGQRLKEPRVDFWTSTIPLILSASDMLHSPLSSLTFLSLLQPFFFFCAP

SEQ ID NO: 92 - protein, 581 aa, Homo sapiens
MSGNWVHPGQILIWAIWVLAAPTKGPSAEGPQRNTRLGWIQGKQVTVLGSPVPVNVFLGVPFAAPPLGSLRFTNPQPASPWDNLREATSYPNLCLQNSEWLLLDQHMLKVHYPKFGVSEDCLYLNIYAPAHADTGSKLPVLVWFPGGAFKTGSASIFDGSALAAYEDVLVVVVQYRLGIFGFFTTWDQHAPGNWAFKDQVAALSWVQKNIEFFGGDPSSVTIFGESAGAISVSSLILSPMAKGLFHKAIMESGVAIIPYLEAHDYEKSEDLQVVAHFCGNNASDSEALLRCLRTKPSKELLTLSQKTKSFTRVVDGAFFPNEPLDLLSQKAFKAIPSIIGVNNHECGFLLPMVRILAVHTATPSNRDAALASTAGHFHRRHQHIPPQYLHLVANEYFHDKHSLTEIRDSLLDLLGDVFFVVPALITARYHRDAGAPVYFYEFRHRPQCFEDTKPAFVKADHADEVRFVFGGAFLKGDIVMFEGATEEEKLLSRKMMKYWATFARTGNPNGNDLSLWPAYNLTEQYLQLDLNMSLGQRLKEPRRDVWVTGYPQPWKAAIIQNKKPRSQILGIKGRISNAKKK

SEQ ID NO: 93 - protein, 356 aa, Homo sapiens
MEDDLSWRSWSLLCMEILSPMAKGLFHKAIMESGVAIIPYLEAHDYEKSEDLQVVAHFCGNNASDSEALLRCLRTKPSKELLTLSQKTKSFTRVVDGAFFPNEPLDLLSQKAFKAIPSIIGVNNHECGFLLPMKEAPEILSGSNKSLALHLIQNILHIPPQYLHLVANEYFHDKHSLTEIRDSLLDLLGDVFFVVPALITARYHRDAGAPVYFYEFRHRPQCFEDTKPAFVKADHADEVRFVFGGAFLKGDIVMFEGATEEEKLLSRKMMKYWATFARTGNPNGNDLSLWPAYNLTEQYLQLDLNMSLGQRLKEPRVDFWTSTIPLILSASDMLHSPLSSLTFLSLLQPFFFFCAP

SEQ ID NO: 94 - protein, 591 aa, Homo sapiens
MDTPRGIGTFVVWDYVVFAGMLVISAAIGIYYAFAGGGQQTSKDFLMGGRRMTAVPVALSLTASFMSAVTVLGTPSEVYRFGAIFSIFAFTYFFVVVISAEVFLPVFYKLGITSTYEYLELRFNKCVRLCGTVLFIVQTILYTGIVIYAPALALNQVTGFDLWGAVVATGVVCTFYCTLGGLKAVIWTDVFQIGIMVAGFASVIIQAVVMQGGISTILNDAYDGGRLNFWNFNPNPLQRHTFWTIIIGGTFTWTSIYGVNQSQVQRYISCKSRFQAKLSLYINLVGLWAILTCSVFCGLALYSRYHDCDPWTAKKVSAPDQLMPYLVLDILQDYPGLPGLFVACAYSGTLSTVSSSINALAAVTVEDLIKPYFRSLSERSLSWISQGMSVVYGALCIGMAALASLMGALLQAALSVFGMVGGPLMGLFALGILVPFANSIGALVGLMAGFAISLWVGIGAQIYPPLPERTLPLHLDIQGCNSTYNETNLITTTEMPFTTSVFQIYNVQRTPLMDNWYSLSYLYFSTVGTLVTLLVGILVSLSTGNYLNISIKKHVLSYKSHPVEDGGTDNPAFNHIELNSDQSGKSNGTRL

SEQ ID NO: 95 - protein, 594 aa, Homo sapiens
MDTPRGIGTFVVWDYVVFAGMLVISAAIGIYYAFAGGGQQTSKDFLMGGRRMTAVPVALSLTASFMSAVTVLGTPSEVYRFGAIFSIFAFTYFFVVVISAEVFLPVFYKLGITSTYEVRGRVGWDHAGRGGRGLCRPLEAFSWGQTVTATSKSLPVHLTGFDLWGAVVATGVVCTFYCTLGGLKAVIWTDVFQIGIMVAGFASVIIQAVVMQGGISTILNDAYDGGRLNFWNFNPNPLQRHTFWTIIIGGTFTWTSIYGVNQSQVQRYISCKSRFQAKLSLYINLVGLWAILTCSVFCGLALYSRYHDCDPWTAKKVSAPDQLMPYLVLDILQDYPGLPGLFVACAYSGTLSTVSSSINALAAVTVEDLIKPYFRSLSERSLSWISQGMSVVYGALCIGMAALASLMGALLQAALSVFGMVGGPLMGLFALGILVPFANSIGALVGLMAGFAISLWVGIGAQIYPPLPERTLPLHLDIQGCNSTYNETNLITTTEMPFTTSVFQIYNVQRTPLMDNWYSLSYLYFSTVGTLVTLLVGILVSLSTGSEQPSKGQSTVSRCHTVLVHCVSLHSGSASAFHFTVLLPQWSWCQWSSK

SEQ ID NO: 96 - protein, 384 aa, Homo sapiens
MVAEVDSMPAASSVKKPFVLRSKMGKWCRHCFPCCRGSGKSNVGTSGDQDDSTMKTLRSKMGKWCCHCFPCCRGSGKSNVGTSGDHDDSAMKTLRSKMGKWCCHCFPCCRGSGKSNVGAWGDYDDSAFVEPRYHVRREDLDKLHRAAWWGKVARKDLIVMLRDTDVNKQDKQKRTALHLASANGNSGVVKLLLDRRCQLNVLDNKKRTALTKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNK

SEQ ID NO: 97 - protein, 280 aa, Homo sapiens
MRGVSCLQVLLLLVLGAAGTQGRKSAACGQPRMSSRIVGGRDGRDGEWPWQASIQHRGAHVCGGSLIAPQWVLTAAHCFPRRALPAEYRVRLGALRLGSTSPRTLSVPVRRVLLPPDYSEDGARGDLALLQLRRPVPLSARVQPVCLPVPGARPPPGTPCRVTGWGSLRPGVPLPEWRPLQGVRVPLLDSRTCDGLYHVGADVPQAERIVLPGSLCAGYPQGHKDACQGDSGGPLTCLQSGSWVLVGVVSWGKGCALPNRPGVYTSVATYSPWIQARVSF

SEQ ID NO: 98 - protein, 282 aa, Homo sapiens
MRGVSCLQVLLLLVLGPQPTLTAPGPFLACGQPRMSSRIVGGRDGRDGEWPWQASIQHRGAHVCGGSLIAPQWVLTAAHCFPRRALPAEYRVRLGALRLGSTSPRTLSVPVRRVLLPPDYSEDGARGDLALLQLRRPVPLSARVQPVCLPVPGARPPPGTPCRVTGWGSLRPGVPLPEWRPLQGVRVPLLDSRTCDGLYHVGADVPQAERIVLPGSLCAGYPQGHKDACQGDSGGPLTCLQSGSWVLVGVVSWGKGCALPNRPGVYTSVATYSPWIQARVSF

SEQ ID NO: 99 - protein, 279 aa, Homo sapiens
MRGVSCLQVLLLLVLGAAGTQGRKSAACGQPRMSSRIVGGRDGRDGEWPWQASIQHRGAHVCGGSLIAPQWVLTAAHCFPRRALPAEYRVRLGALRLGSTSPRTLSVPVRRVLLPPDYSEDGARGDLALLQLRRPVPLSARVQPVCLPVPGARPPPGTPCRVTGWGSLRPGVPLPEWRPLQGVRVPLLDSRTCDGLYHVGADVPQAERIVLPGSLCAGYPQGHKDACQVCTQPPQPPESPPCAQHPPSLNSRTQDIPTQAQDPGLQPRGTTPGVWNPEN

SEQ ID NO: 100 - protein, 553 aa, Homo sapiens
MVDMGALDNLIANTAYLQARKPSDCDSKELQRRRRSLALPGLQGCAELRQKLSLNFHSLCEQQPIGRRLFRDFLATVPTFRKAATFLEDVQNWELAEEGPTKDSALQGLVATCASAPAPGNPQPFLSQAVATKCQAATTEEERVAAVTLAKAEAMAFLQEQPFKDFVTSAFYDKFLQWKLFEMQPVSDKYFTEFRVLGKGGFGEVCAVQVKNTGKMYACKKLDKKRLKKKGGEKMALLEKEILEKVSSPFIVSLAYAFESKTHLCLVMSLMNGGDLKFHIYNVGTRGLDMSRVIFYSAQIACGMLHLHELGIVYRDMKPENVLLDDLGNCRLSDLGLAVEMKGGKPITQRAGTNGYMAPEILMEKVSYSYPVDWFAMGCSIYEMVAGRTPFKDYKEKVSKEDLKQRTLQDEVKFQHDNFTEEAKDICRLFLAKKPEQRLGSREKSDDPRKHHFFKTINFPRLEAGLIEPPFVPDPSVVYAKDIAEIDDFSEVRGVEFDDKDKQFFKNFATGAVPIAWQEEIIETGLFEELNDPNRPTGCEEGNSSKSGVCLLL

SEQ ID NO: 101 - protein, 442 aa, Homo sapiens
MQWALKVGHFAQGTLPASMPPFLITLFLFHSCCLRANGHLREGMTLLKTEFALHLYQSVAACRNETNFVISPAGVSLPLEILQFGAEGSTGQQLADALGYTVHDKRVKDFLHAVYATLPTSSQGTEMELACSLFVQVGTPLSPCFVEHVSWWANSSLEPADLSEPNSTAIQTSEGASRETAGGGPSEGPGGWPWEQVSAAFAQLVLVSTMSFQGTWRKRFSSTDTQILPFTCAYGLVLQVPMMHQTTEVNYGQFQDTAGHQVGVLELPYLGSAVSLFLVLPRDKDTPLSHIEPHLTASTIHLWTTSLRRARMDVFLPRFRIQNQFNLKSILNSWGVTDLFDPLKANLKGISGQDGFYVSEAIHKAKIEVLEEGTKASGATALLLLKRSRIPIFKADRPFIYFLREPNTGITVFFDRIQIIYQCLSSNKGSFVHYPLKNKHSF

SEQ ID NO: 102 - protein, 1027 aa, Homo sapiens
MLVVERVMVLPIGFPLGVSDDSTLHGPIFIQEPSPVMFPLDSEEKKVKLNCEVKGNPKPHIRWKLNGTDVDTGMDFRYSVVEGSLLINNPNKTQDAGTYQCTATNSFGTIVSREAKLQFAYLDNFKTRTRSTVSVRRGQGMVLLCGPPPHSGELSYAWIFNEYPSYQDNRRFVSQETGNLYIAKVEKSDVGNYTCVVTNTVTNHKVLGPPTPLILRNDGVMGEYEPKIEVQFPETVPTAKGATVKLECFALGNPVPTIIWRRADGKPIARKARRHKSNGILEIPNFQQEDAGLYECVAENSRGKNVARGQLTFYAQPNWIQKINDIHVAMEENVFWECKANGRPKPTYKWLKNGEPLLTRDRIQIEQGTLNITIVNLSDAGMYQCLAENKHGVIFSNAELSVIAVGPDFSRTLLKRVTLVKVGGEVVIECKPKASPKPVYTWKKGRDILKENERITISEDGNLRIINVTKSDAGSYTCIATNHFGTASSTGNLVVKDPTRVMVPPSSMDVTVGESIVLPCQVTHDHSLDIVFTWSFNGHLIDFDRDGDHFERVGGQDSAGDLMIRNIQLKHAGKYVCMVQTSVDRLSAAADLIVRGPPGPPEAVTIDEITDTTAQLSWRPGPDNHSPITMYVIQARTPFSVGWQAVSTVPELIDGKTFTATVVGLNPWVEYEFRTVAANVIGIGEPSRPSEKRRTEEALPEVTPANVSGGGGSKSELVITWETVPEELQNGRGFGYVVAFRPYGKMIWMLTVLASADASRYVFRNESVHPFSPFEVKVGVFNNKGEGPFSPTTVVYSAEEEPTKPPASIFARSLSATDIEVFWASPLEKNRGRIQGYEVKYWRHEDKEENARKIRTVGNQTSTKITNLKGSVLYHLAVKAYNSAGTGPSSATVNVTTRKPPPSQPPGNIIWNSSDSKIILNWDQVKALDNESEVKGYKVLYRWNRQSSTSVIETNKTSVELSLPFDEDYIIEIKPFSDGGDGSSSEQIRIPKISNAYARGSGASTSNACTLSAISTIMISLTARSSL

SEQ ID NO: 103 - DNA, 1569 bp, Homo sapiens
atggatttcctgctgctcggtctctgtctatactggctgctgaggaggccctcgggggtggtcttgtgtctgctgggggcctgctttcagatgctgcccgccgcccccagcgggtgcccgcagctgtgccggtgcgaggggcggctgctgtactgcgaggcgctcaacctcaccgaggcgccccacaacctgtccggcctgctgggcttgtccctgcgctacaacagcctctcggagctgcgcgccggccagttcacggggttaatgcagctcacgtggctctatctggatcacaatcacatctgctccgtgcagggggacgcctttcagaaactgcgccgagttaaggaactcacgctgagttccaaccagatcacccaactgcccaacaccaccttccggcccatgcccaacctgcgcagcgtggacctctcgtacaacaagctgcaggcgctcgcgcccgacctcttccacgggctgcggaagctcaccacgctgcatatgcgggccaacgccatccagtttgtgcccgtgcgcatcttccaggactgccgcagcctcaagtttctcgacatcggatacaatcagctcaagagtctggcgcgcaactctttcgccggcttgtttaagctcaccgagctgcacctcgagcacaacgacttggtcaaggtgaacttcgcccacttcccgcgcctcatctccctgcactcgctctgcctgcggaggaacaaggtggccattgtggtcagctcgctggactgggtttggaacctggagaaaatggacttgtcgggcaacgagatcgagtacatggagccccatgtgttcgagaccgtgccgcacctgcagtccctgcagctggactccaaccgcctcacctacatcgagccccggatcctcaactcttggaagtccctgacaagcatcaccctggccgggaacctgtgggattgcgggcgcaacgtgtgtgccctagcctcgtggctcaacaacttccaggggcgctacgatggcaacttgcagtgcgccagcccggagtacgcacagggcgaggacgtcctggacgccgtgtacgccttccacctgtgcgaggatggggccgagcccaccagcggccacctgctctcggccgtcaccaaccgcagtgatctggggccccctgccagctcggccaccacgctcgcggacggcggggaggggcagcacgacggcacattcgagcctgccaccgtggctcttccaggcggcgagcacgccgagaacgccgtgcagatccacaaggtggtcacgggcaccatggccctcatcttctccttcctcatcgtggtcctggtgctctacgtgtcctggaagtgtttcccagccagcctcaggcagctcagacagtgctttgtcacgcagcgcaggaagcaaaagcagaaacagaccatgcatcagatggctgccatgtctgcccaggaatactacgttgattacaaaccgaaccacattgagggagccctggtgatcatcaacgagtatggctcgtgtacctgccaccagcagcccgcgagggaatgcgaggtgtga

SEQ ID NO: 104 - protein, 522 aa, Homo sapiens
MDFLLLGLCLYWLLRRPSGVVLCLLGACFQMLPAAPSGCPQLCRCEGRLLYCEALNLTEAPHNLSGLLGLSLRYNSLSELRAGQFTGLMQLTWLYLDHNHICSVQGDAFQKLRRVKELTLSSNQITQLPNTTFRPMPNLRSVDLSYNKLQALAPDLFHGLRKLTTLHMRANAIQFVPVRIFQDCRSLKFLDIGYNQLKSLARNSFAGLFKLTELHLEHNDLVKVNFAHFPRLISLHSLCLRRNKVAIVVSSLDWVWNLEKMDLSGNEIEYMEPHVFETVPHLQSLQLDSNRLTYIEPRILNSWKSLTSITLAGNLWDCGRNVCALASWLNNFQGRYDGNLQCASPEYAQGEDVLDAVYAFHLCEDGAEPTSGHLLSAVTNRSDLGPPASSATTLADGGEGQHDGTFEPATVALPGGEHAENAVQIHKVVTGTMALIFSFLIVVLVLYVSWKCFPASLRQLRQCFVTQRRKQKQKQTMHQMAAMSAQEYYVDYKPNHIEGALVIINEYGSCTCHQQPARECEV

SEQ ID NO: 105 - DNA, 870 bp, Homo sapiens
atgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 106 - protein, 289 aa, Homo sapiens
MFVLLYVTSFAICASGQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 107 - DNA, 1047 bp, Homo sapiens
atgtcccttgcttcaggccctggccctgggtggttactcttttcctttggaatggggctggtatcagggtcaaagtgtccaaataattgtctgtgtcaagcccaagaagtaatctgcacagggaagcagttaaccgaatacccccttgacatacccctgaacacccggaggctgttcctgaacgagaacagaatcactagtttgccagcaatgcatctaggactcctcagtgaccttgtttatttggactgtcagaacaaccggattcgagaggtgatggattataccttcatcggggtcttcaaactcatctaccttgacctcagctccaacaacctaacctcgatctccccattcactttctcggtgctcagcaacctggtgcagctgaacattgccaacaaccctcacctgttatcgcttcacaagttcacctttgccaacaccacctctttgaggtacctggacctcagaaataccggcttgcagaccctggacagtgctgccttataccacctcactactctggagaccctgtttctgagtggaaacccctggaagtgcaactgctctttcctggacttcgccatcttcttaatagtgttccatatggacccctcagatgatctaaatgccacatgtgtggagcccacagagctgacagggtggcccatcacccgggtggggaacccactccgatacatgtgcatcacgcacctggaccacaaagactacatcttcctgctgctcatcggcttctgcatcttcgccgcgggaactgtggctgcctggctcacaggtgtgtgtgctgtgctctaccagaacacccgccacaagtcgagtgaagaagatgaggacgaggccgggactagggtggaagtcagcaggcggatttttcaacccagacgagctcggtccaggagttccctcagcttatttagttgccagagaccactatcttatgtgcctcccccaggctccctgctttctctcttgccctccccatcccaccaccttggagctgtcatagagattgaaaccttctag

SEQ ID NO: 108 - protein, 348 aa, Homo sapiens
MSLASGPGPGWLLFSFGMGLVSGSKCPNNCLCQAQEVICTGKQLTEYPLDIPLNTRRLFLNENRITSLPAMHLGLLSDLVYLDCQNNRIREVMDYTFIGVFKLIYLDLSSNNLTSISPFTFSVLSNLVQLNIANNPHLLSLHKFTFANTTSLRYLDLRNTGLQTLDSAALYHLTTLETLFLSGNPWKCNCSFLDFAIFLIVFHMDPSDDLNATCVEPTELTGWPITRVGNPLRYMCITHLDHKDYIFLLLIGFCIFAAGTVAAWLTGVCAVLYQNTRHKSSEEDEDEAGTRVEVSRRIFQPRRARSRSSLSLFSCQRPLSYVPPPGSLLSLLPSPSHHLGAVIEIETF

SEQ ID NO: 109 - DNA, 1281 bp, Homo sapiens
atggcgcgcaggacagagccccccgacgggggctggggatgggtggtggtgctctcagcgttcttccagtcggcgcttgtgtttggggtgctccgctcctttggggtcttcttcgtggagtttgtggcggcgtttgaggagcaggcagcgcgcgtctcctggatcgcctccataggaatcgcggtgcagcagtttgggagcccggtaggcagtgccctgagcacgaagttcgggcccaggcccgtggtgatgactggaggcatcttggctgcgctggggatgctgctcgcctcttttgctacttccttgacccacctatacctgagtattgggttgctgtcaggctctggctgggctttgaccttcgctccgaccctggcctgcctgtcctgttatttctctcgccgacgatccctggccaccgggctggcactgacaggcgtgggcctctcctccttcacatttgccccctttttccagtggctgctcagccactacgcctggagggggtccctgctgctggtgtctgccctctccctccacctagtggcctgtggtgctctcctccgcccaccctccctggctgaggaccctgctgtgggtggtcccagggcccaactcacctctctcctccatcatggccccttcctccgttacactgttgccctcaccctgatcaacactggctacttcattccctacctccacctggtggcccatctccaggacctggattgggacccactacctgctgccttcctactctcagttgttgctatttctgacctcgtggggcgtgtggtctccggatggctgggagatgcagtcccagggcctgtgacacgactcctgatgctctggaccaccttgactggggtgtcactagccctgttccctgtagctcaggctcccacagccctggtggctctggctgtggcctacggcttcacatcaggggctctggccccactggccttctccgtgctgcctgaactaatagggactagaaggatttactgtggcctgggactgttgcagatgatagagagcatcggggggctgctggggcctcctctctcaggctacctccgggatgtgtcaggcaactacacggcttcttttgtggtggctggggccttccttctttcagggagtggcattctcctcaccctgccccacttcttctgcttctcaactactacctccgggcctcaggaccttgtaacagaagcactagatactaaagttcccctacccaaggaggggctggaagaggactga

SEQ ID NO: 110 - protein, 426 aa, Homo sapiens
MARRTEPPDGGWGWVVVLSAFFQSALVFGVLRSFGVFFVEFVAAFEEQAARVSWIASIGIAVQQFGSPVGSALSTKFGPRPVVMTGGILAALGMLLASFATSLTHLYLSIGLLSGSGWALTFAPTLACLSCYFSRRRSLATGLALTGVGLSSFTFAPFFQWLLSHYAWRGSLLLVSALSLHLVACGALLRPPSLAEDPAVGGPRAQLTSLLHHGPFLRYTVALTLINTGYFIPYLHLVAHLQDLDWDPLPAAFLLSVVAISDLVGRVVSGWLGDAVPGPVTRLLMLWTTLTGVSLALFPVAQAPTALVALAVAYGFTSGALAPLAFSVLPELIGTRRIYCGLGLLQMIESIGGLLGPPLSGYLRDVSGNYTASFVVAGAFLLSGSGILLTLPHFFCFSTTTSGPQDLVTEALDTKVPLPKEGLEED

(19) United States
(12) Patent Application Publication
(10) Pub. No.: US 2004/0091969 A1
(43) Pub. Date: May 13, 2004

(54) Novel compounds

(76) Inventors: Pankaj Agarwal, Audubon, PA (US); Marian Birkeland, King of Prussia, PA (US); John P Cogswell, Research Triangle Park, NC (US); Karen S Kabnick, Lafayette Hill, PA (US); Ying-Ta Lai, Wugu Shiang Taipei (TW); Safia K Rivzi, Philadelphia, PA (US); Shelby A Martensen, Research Triangle Park, NC (US); Randall F Smith, Lafayette Hill, PA (US); Jay C Strum, Research Triangle Park, NC (US); Qing Xie, Horsham, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/451,168

(22) PCT Filed: Dec. 17, 2001
(86) PCT No.: PCT/US01/49232

(51) Int. Cl. C07H 21/04; C12N 1/21; C07K 14/00

(52) U.S. Cl. 435/69.3; 530/350; 435/320.1; 435/252.3; 536/23.7

(57) ABSTRACT

Polypeptides and polynucleotides of the genes set forth in Table I and methods for producing such polypeptides by recombinant techniques are disclosed. Also disclosed are methods for utilizing polypeptides and polynucleotides of the genes set forth in Table I in diagnostic assays.

### FIELD OF INVENTION

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides. The polynucleotides and polypeptides of the present invention also relate to proteins with signal sequences which allow them to be secreted extracellularly or membrane-associated (hereinafter often referred collectively as secreted proteins or secreted polypeptides).

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superseding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

Proteins and polypeptides that are naturally secreted into blood, lymph and other body fluids, or secreted into the cellular membrane are of primary interest for pharmaceutical research and development. The reason for this interest is the relative ease to target protein therapeutics into their place of action (body fluids or the cellular membrane). The natural pathway for protein secretion into extracellular space is the endoplasmic reticulum in eukaryotes and the inner membrane in prokaryotes (Palade, 1975, Science, 189, 347; Milstein, Brownlee, Harrison, and Mathews, 1972, Nature New Biol., 239, 117; Blobel, and Dobberstein, 1975, J. Cell. Biol., 67, 835). On the other hand, there is no known natural pathway for exporting a protein from the exterior of the cells into the cytosol (with the exception of pinocytosis, a mechanism of snake venom toxin intrusion into cells). Therefore targeting protein therapeutics into cells poses extreme difficulties.

The secreted and membrane-associated proteins include but are not limited to all peptide hormones and their receptors (including but not limited to insulin, growth hormones, chemokines, cytokines, neuropeptides, integrins, kallikreins, lamins, melanins, natriuretic hormones, neuropsin, neurotropins, pituitiary hormones, pleiotropins, prostaglandins, secretogranins, selectins, thromboglobulins, thymosins), the breast and colon cancer gene products, leptin, the obesity gene protein and its receptors, serum albumin, superoxide dismutase, spliceosome proteins, 7TM (transmembrane) proteins also called as G-protein coupled receptors, immunoglobulins, several families of serine proteinases (including but not limited to proteins of the blood coagulation cascade, digestive enzymes), deoxyribonuclease I, etc.

Therapeutics based on secreted or membrane-associated proteins approved by FDA or foreign agencies include but are not limited to insulin, glucagon, growth hormone, chorionic gonadotropin, follicle stimulating hormone, luteinizing hormone, calcitonin, adrenocorticotropic hormone (ACTH), vasopressin, interleukines, interferones, immunoglobulins, lactoferrin (diverse products marketed by several companies), tissue-type plasminogen activator (Alteplase by Genentech), hyaulorindase (Wydase by Wyeth-Ayerst), dornase alpha (Pulmozyme\ by Genentech), Chymodiactin (chymopapain by Knoll), alglucerase (Ceredase by Genzyme), streptokinase (Kabikinase by Pharmacia) (Streptase by Astra), etc. This indicates that secreted and membrane-associated proteins have an established, proven history as therapeutic targets. Clearly, there is a need for identification and characterization of further secreted and membrane-associated proteins which can play a role in preventing, ameliorating or correcting dysfunction or disease, including but not limited to diabetes, breast-, prostate-, colon cancer and other malignant tumors, hyper- and hypotension, obesity, bulimia, anorexia, growth abnormalities, asthma, manic depression, dementia, delirium, mental retardation, Huntington's disease, Tourette's syndrome, schizophrenia, growth, mental or sexual development disorders, and dysfunctions of the blood cascade system including those leading to stroke. The proteins of the present invention which include the signal sequences are also useful to further elucidate the mechanism of protein transport which at present is not entirely understood, and thus can be used as research tools.

### SUMMARY OF THE INVENTION

The present invention relates to particular polypeptides and polynucleotides of the genes set forth in Table I, including recombinant materials and methods for their production. Such polypeptides and polynucleotides are of interest in relation to methods of treatment of certain diseases, including, but not limited to, the diseases set forth in Tables m and V, hereinafter referred to as “diseases of the invention”. In a further aspect, the invention relates to methods for identifying agonists and antagonists (e.g., inhibitors) using the materials provided by the invention, and treating conditions associated with imbalance of polypeptides and/or polynucleotides of the genes set forth in Table I with the identified compounds. In still a further aspect, the invention relates to diagnostic assays for detecting diseases associated with inappropriate activity or levels the genes set forth in Table I. Another aspect of the invention concerns a polynucleotide comprising any of the nucleotide sequences set forth in the Sequence Listing and a polypeptide comprising a polypeptide encoded by the nucleotide sequence. In another aspect, the invention relates to a polypeptide comprising any of the polypeptide sequences set forth in the Sequence Listing and recombinant materials and methods for their production. Another aspect of the invention relates to methods for using such polypeptides and polynucleotides. Such uses include the treatment of diseases, abnormalities and disorders (hereinafter simply referred to as diseases) caused by abnormal expression, production, function and or metabolism of the genes of this invention, and such diseases are readily apparent by those skilled in the art from the homology to other proteins disclosed for each attached sequence. In still another aspect, the invention relates to methods to identify agonists and antagonists using the materials provided by the invention, and treating conditions associated with the imbalance with the identified compounds. Yet another aspect of the invention relates to diagnostic assays for detecting diseases associated with inappropriate activity or levels of the secreted proteins of the present invention.

### DESCRIPTION OF THE INVENTION

In a first aspect, the present invention relates to polypeptides the genes set forth in Table I. Such polypeptides include:

(a) an isolated polypeptide encoded by a polynucleotide comprising a sequence set forth in the Sequence Listing, herein when referring to polynucleotides or polypeptides of the Sequence Listing, a reference is also made to the Sequence Listing referred to in the Sequence Listing;

(b) an isolated polypeptide comprising a polypeptide sequence having at least 95%, 96%, 97%, 98%, or 99% identity to a polypeptide sequence set forth in the Sequence Listing;

(c) an isolated polypeptide comprising a polypeptide sequence set forth in the Sequence Listing;

(d) an isolated polypeptide having at least 95%, 96%, 97%, 98%, or 99% identity to a polypeptide sequence set forth in the Sequence Listing;

(e) a polypeptide sequence set forth in the Sequence Listing; and

(f) an isolated polypeptide having or comprising a polypeptide sequence that has an Identity Index of 0.95, 0.96, 0.97, 0.98, or 0.99 compared to a polypeptide sequence set forth in the Sequence Listing;

(g) fragments and variants of such polypeptides in (a) to (f).

Polypeptides of the present invention are believed to be members of the gene families set forth in Table II. They are therefore of therapeutic and diagnostic interest for the reasons set forth in Tables III and V. The biological properties of the polypeptides and polynucleotides of the genes set forth in Table I are hereinafter referred to as “the biological activity” of polypeptides and polynucleotides of the genes set forth in Table I. Preferably, a polypeptide of the present invention exhibits at least one biological activity of the genes set forth in Table I.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from an amino acid sequence set forth in the Sequence Listing, or an isolated polypeptide comprising an amino acid sequence having at least 30, 50 or 100 contiguous amino acids truncated or deleted from an amino acid sequence set forth in the Sequence Listing. Preferred fragments are biologically active fragments that mediate the biological activity of polypeptides and polynucleotides of the genes set forth in Table I, including those with a similar activity or an improved activity, or with a decreased undesirable activity. Also preferred are those fragments that are antigenic or immunogenic in an animal, especially in a human.

Fragments of a polypeptide of the invention may be employed for producing the corresponding full-length polypeptide by peptide synthesis; therefore, these variants may be employed as intermediates for producing the full-length polypeptides of the invention. A polypeptide of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to polynucleotides of the genes set forth in Table I. Such polynucleotides include:

(a) an isolated polynucleotide comprising a polynucleotide sequence having at least 95%, 96%, 97%, 98%, or 99% identity to a polynucleotide sequence set forth in the Sequence Listing;

(b) an isolated polynucleotide comprising a polynucleotide set forth in the Sequence Listing;

(c) an isolated polynucleotide having at least 95%, 96%, 97%, 98%, or 99% identity to a polynucleotide set forth in the Sequence Listing;

(d) an isolated polynucleotide set forth in the Sequence Listing;

(e) an isolated polynucleotide comprising a polynucleotide sequence encoding a polypeptide sequence having at least 95%, 96%, 97%, 98%, or 99% identity to a polypeptide sequence set forth in the Sequence Listing;

(f) an isolated polynucleotide comprising a polynucleotide sequence encoding a polypeptide set forth in the Sequence Listing;

(g) an isolated polynucleotide having a polynucleotide sequence encoding a polypeptide sequence having at least 95%, 96%, 97%, 98%, or 99% identity to a polypeptide sequence set forth in the Sequence Listing;

(h) an isolated polynucleotide encoding a polypeptide set forth in the Sequence Listing;

(i) an isolated polynucleotide having or comprising a polynucleotide sequence that has an Identity Index of 0.95, 0.96, 0.97, 0.98, or 0.99 compared to a polynucleotide sequence set forth in the Sequence Listing;

(j) an isolated polynucleotide having or comprising a polynucleotide sequence encoding a polypeptide sequence that has an Identity Index of 0.95, 0.96, 0.97, 0.98, or 0.99 compared to a polypeptide sequence set forth in the Sequence Listing; and polynucleotides that are fragments and variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from a sequence set forth in the Sequence Listing, or an isolated polynucleotide comprising a sequence having at least 30, 50 or 100 contiguous nucleotides truncated or deleted from a sequence set forth in the Sequence Listing.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise an amino acid sequence set forth in the Sequence Listing and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides that are RNA transcripts of the DNA sequences of the present invention. Accordingly, there is provided an RNA polynucleotide that:

(a) comprises an RNA transcript of the DNA sequence encoding a polypeptide set forth in the Sequence Listing;

(b) is a RNA transcript of a DNA sequence encoding a polypeptide set forth in the Sequence Listing;

(c) comprises an RNA transcript of a DNA sequence set forth in the Sequence Listing; or

(d) is a RNA transcript of a DNA sequence set forth in the Sequence Listing; and RNA polynucleotides that are complementary thereto.

The polynucleotide sequences set forth in the Sequence Listing show homology with the polynucleotide sequences set forth in Table II. A polynucleotide sequence set forth in the Sequence Listing is a cDNA sequence that encodes a polypeptide set forth in the Sequence Listing. A polynucleotide sequence encoding a polypeptide set forth in the Sequence Listing may be identical to a polypeptide encoding a sequence set forth in the Sequence Listing or it may be a sequence other than a sequence set forth in the Sequence Listing, which, as a result of the redundancy (degeneracy) of the genetic code, also encodes a polypeptide set forth in the Sequence Listing. A polypeptide of a sequence set forth in the Sequence Listing is related to other proteins of the gene families set forth in Table II, having homology and/or structural similarity with the polypeptides set forth in Table II. Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of the genes set forth in Table I.

Polynucleotides of the present invention may be obtained using standard cloning and screening techniques from a cDNA library derived from mRNA from the tissues set forth in Table IV (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. A polynucleotide may also contain non-coding 5′ and 3′ sequences, such as transcribed, non-translated sequences, splicing and polyadenylation signals, ribosome binding sites and sequences, that stabilize mRNA.

Polynucleotides that are identical, or have sufficient identity to a polynucleotide sequence set forth in the Sequence Listing, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from other species) that have a high sequence similarity to sequences set forth in the Sequence Listing, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from other species, may be obtained by a process comprising the steps of screening a library under stringent hybridization conditions with a labeled probe having a sequence set forth in the Sequence Listing or a fragment thereof, preferably of at least 15 nucleotides; and isolating full-length cDNA and genomic clones containing the polynucleotide sequence set forth in the Sequence Listing. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence set forth in the Sequence Listing or a fragment thereof, preferably of at least 15 nucleotides.

The skilled artisan will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the Marathon (trade mark) technology (Clontech Laboratories Inc.) for example, have significantly simplified the search for longer cDNAs. In the Marathon (trade mark) technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al.(ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction or infection.

Representative examples of appropriate hosts include bacterial cells, such as Streptococci, Staplrylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of a gene is characterized by the polynucleotides set forth in the Sequence Listing in the cDNA or genomic sequence and which is associated with a dysfunction. Will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences of the genes set forth in Table I. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85:4397-4401).

An array of oligonucleotides probes comprising polynucleotide sequences or fragments thereof of the genes set forth in Table I can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising:

(a) a polynucleotide of the present invention, preferably the nucleotide sequence set forth in the Sequence Listing, or a fragment or an RNA transcript thereof;

(b) a nucleotide sequence complementary to that of (a);

(c) a polypeptide of the present invention, preferably the polypeptide set forth in the Sequence Listing or a fragment thereof; or

(d) an antibody to a polypeptide of the present invention, preferably to the polypeptide set forth in the Sequence Listing.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localisation studies. The sequences set forth in the Sequence Listing are specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localisations for a genomic sequence (gene fragment etc.) can be determined using Radiation Hybrid (RH) Mapping (Walter, M. Spillett, D., Thomas, P., Weissenbach, J., and Goodfellow, P., (1994) A method for constructing radiation hybrid maps of whole genomes, Nature Genetics 7, 22-28). A number of RH panels are available from Research Genetics (Huntsville, Ala., USA) e.g. the GeneBridge4 RH panel (Hum Mol Genet 1996 March;5(3):339-46 A radiation hybrid map of the human genome. Gyapay G, Schmitt K, Fizames C, Jones H, Vega-Czarny N, Spillett D, Muselet D, Prud'Homme J F, Dib C, Auffray C, Morissette J, Weissenbach J, Goodfellow P N). To determine the chromosomal location of a gene using this panel, 93 PCRs are performed using primers designed from the gene of interest on RH DNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at http://www.genome.wi.mit.edu/.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hydridization techniques to clones arrayed on a grid, such as cDNA microarray hybridization (Schena et al, Science, 270, 467-470, 1995 and Shalon et al, Genome Res, 6, 639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intramuscular, intravenous, or intra-dermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2):Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring an activity of the genes set forth in Table I in the mixture, and comparing activity of the mixture of the genes set forth in Table I to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and polypeptide of the genes set forth in Table I, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and the genes set forth in Table I. The art of constructing transgenic animals is well established. For example, the genes set forth in Table I may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention

Screening kits for use in the above described methods form a further aspect of the present invention. Such screening kits comprise:

(a) a polypeptide of the present invention;

(b) a recombinant cell expressing a polypeptide of the present invention;

(c) a cell membrane expressing a polypeptide of the present invention; or

(d) an antibody to a polypeptide of the present invention;

which polypeptide is preferably that set forth in the Sequence Listing.

It will be appreciated that in any such kit, (a), (b), (c) or (d) may comprise a substantial component.

Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an

Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Secreted protein activity or secreted polypeptide activity” or “biological activity of the secreted protein or secreted polypeptide” refers to the metabolic or physiologic function of said secreted protein including similar activities or improved activities or these activities with decreased undesirable side-effects. Also included are antigenic and immunogenic activities of said secreted protein.

“Secreted protein gene” refers to a polynucleotide comprising any of the attached nucleotide sequences or allelic variants thereof and/or their complements.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663,48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence set forth in the Sequence Listing.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448,1988, available as part of the Wisconsin Sequence Analysis Package).

Preferably, the BLOSUM62 amino acid substitution matrix (Henikoff S and Henikoff J G, Proc. Nat. Acad. Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis nutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutantdis for other values of the Identity Index, for instance 0.96, 0.97, 0.98 and 0.99.

The relationship between the number of nucleotide or amino acid differences and the Identity Index may be expressed in the following equation:

nₐ<xₐ−(xₐ•I),

in which:

nₐ is the number of nucleotide or amino acid differences,

xₐ is the total number of nucleotides or amino acids in a sequence set forth in the Sequence Listing,

I is the Identity Index,

• is the symbol for the multiplication operator, and

in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

TABLE I

| | | | Corresponding |
|---|---|---|---|
| | GSK | Nucleic Acid | Protein |
| Gene Name | Gene ID | SEQ ID NO's | SEQ ID NO's |
| sbg1571549- | 1571549 | SEQ ID NO: 1 | SEQ ID NO: 56 |
| cystatin-re | | SEQ ID NO: 2 | SEQ ID NO: 57 |
| sbg1558434SRCR | 1558434 | SEQ ID NO: 3 | SEQ ID NO: 58 |
| | | SEQ ID NO: 4 | SEQ ID NO: 59 |
| sbg1546354LRR | 1546354 | SEQ ID NO: 5 | SEQ ID NO: 60 |
| | | SEQ ID NO: 6 | SEQ ID NO: 61 |
| sbg1555869CABP | 1555869 | SEQ ID NO: 7 | SEQ ID NO: 62 |
| | | SEQ ID NO: 8 | SEQ ID NO: 63 |
| sbg1518024NKR | 1518024 | SEQ ID NO: 9 | SEQ ID NO: 64 |
| | | SEQ ID NO: 10 | SEQ ID NO: 65 |
| sbg1525809WNT8 | 1525809 | SEQ ID NO: 11 | SEQ ID NO: 66 |
| sbg1519904cdk | 1519904 | SEQ ID NO: 12 | SEQ ID NO: 67 |
| sbg1521449- | 1521449 | SEQ ID NO: 13 | SEQ ID NO: 68 |
| connexin | | SEQ ID NO: 14 | SEQ ID NO: 69 |
| sbg1522659- | 1522659 | SEQ ID NO: 15 | SEQ ID NO: 70 |
| Siglec | | SEQ ID NO: 16 | SEQ ID NO: 71 |
| sbg1528389ITI | 1528389 | SEQ ID NO: 17 | SEQ ID NO: 72 |
| sbg1526964ISLR | 1526964 | SEQ ID NO: 18 | SEQ ID NO: 73 |
| sbg1539439TSPK | 1539439 | SEQ ID NO: 19 | SEQ ID NO: 74 |
| sbg1571349lipase | 1571349 | SEQ ID NO: 20 | SEQ ID NO: 75 |
| | | SEQ ID NO: 21 | SEQ ID NO: 76 |
| sbg1565539- | 1565539 | SEQ ID NO: 22 | SEQ ID NO: 77 |
| aCONNEXIN | | | |
| sbg1565539- | 1565539 | SEQ ID NO: 23 | SEQ ID NO: 78 |
| bCONNEXIN | | | |
| sbg1563294- | 1563294 | SEQ ID NO: 24 | SEQ ID N0: 79 |
| acadherin | | SEQ ID NO: 25 | SEQ ID NO: 80 |
| sbg1563294- | 1563294 | SEQ ID NO: 26 | SEQ ID NO: 81 |
| bcadherin | | | |
| sbg1563509ECM | 1563509 | SEQ ID NO: 27 | SEQ ID NO: 82 |
| | | SEQ ID NO: 28 | SEQ ID NO: 83 |
| sbg1552159- | 1552159 | SEQ ID NO: 29 | SEQ ID NO: 84 |
| Serprotease | | | |
| sbg1551159- | 1551159 | SEQ ID NO: 30 | SEQ ID NO: 85 |
| thymosinb4 | | | |
| SBh1686954.SOCS | 1686954 | SEQ ID NO: 31 | SEQ ID NO: 86 |
| sbg1548844slit | 1548844 | SEQ ID NO: 32 | SEQ ID NO: 87 |
| sbg1545249GGT | 1545249 | SEQ ID NO: 33 | SEQ ID NO: 88 |
| | | SEQ ID NO: 34 | SEQ ID NO: 89 |
| SBh1813899.Y82017 | 1813899 | SEQ ID NO: 35 | SEQ ID NO: 90 |
| sbg1536324mapkkk | 1536324 | SEQ ID NO: 36 | SEQ ID NO: 91 |
| gsk1810944BrCaAg | 1810944 | SEQ ID NO: 37 | SEQ ID NO: 92 |
| sbg1535914AIP | 1535914 | SEQ ID NO: 38 | SEQ ID NO: 93 |
| | | SEQ ID NO: 39 | SEQ ID NO: 94 |
| sbg1530894aCE | 1530894 | SEQ ID NO: 40 | SEQ ID NO: 95 |
| | | SEQ ID NO: 41 | SEQ ID NO: 96 |
| sbg1530894bCE | 1530894 | SEQ ID NO: 42 | SEQ ID NO: 97 |
| gsk.1813134.NaI | 1813134 | SEQ ID NO: 43 | SEQ ID NO: 98 |
| | | SEQ ID NO: 44 | SEQ ID NO: 99 |
| gsk1811484BrCaAg | 1811484 | SEQ ID NO: 45 | SEQ ID NO: 100 |
| sbg1529984- | 1529984 | SEQ ID NO: 46 | SEQ ID NO: 101 |
| aSproteinase | | SEQ ID NO: 47 | SEQ ID NO: 102 |
| sbg1529984- | 1529984 | SEQ ID NO: 48 | SEQ ID NO: 103 |
| bSproteinase | | | |
| SBGKIN113 | 575482 | SEQ ID NO: 49 | SEQ ID NO: 104 |
| gsk305961GDNa | 305961 | SEQ ID NO: 50 | SEQ ID NO: 105 |
| gsk2402719_{—} | 2402719 | SEQ ID NO: 51 | SEQ ID NO: 106 |
| 2395124BIG2 | and | | |
| | 2395124 | | |
| sbg18525LRRb | 18525 | SEQ ID NO: 52 | SEQ ID NO: 107 |
| SBhACRP30c | 34718 | SEQ ID NO: 53 | SEQ ID NO: 108 |
| sbg123493SLITc | 123493 | SEQ ID NO: 54 | SEQ ID NO: 109 |
| sbg102200MCTc | 102200 | SEQ ID NO: 55 | SEQ ID NO: 110 |

TABLE II

| | | Closest | | Cell |
|---|---|---|---|---|
| | | Polynuclotide by | Closest Polypeptide by | Localization |
| Gene Name | Gene Family | homology | homology | (by homology) |
| sbg1571549cystatin-re | Testatin | SC: AL121894 | Human cystatin-related | Secreted |
| | | Direct submitted | protein, designated | |
| | | (25 OCT. 2000) by | testatin, | |
| | | Sanger Centre, | geneseqp: Y53771 | |
| | | Hinxton, | Filed by (KARO-) | |
| | | Cambridgeshire, | KAROLINSKA | |
| | | CB10 1SA, UK. | INNOVATIONS AB | |
| | | | Publication number and | |
| | | | filing date: | |
| | | | WO9958565-A1, 18 | |
| | | | NOV. 1999 | |
| sbg1558434SRCR | Scavenger | GB: AL160313 | Human KIAA1822 | Membrane- |
| | receptor with | Directly submitted | protein, gi: 14017860 | bound |
| | cysteine-rich | (23 MAY 2000) by | Submitted (27 MAR. | |
| | domain | Genoscope to the | 2001) by Osamu Ohara, | |
| | | EMBL/GenBank/ | Kazusa DNA Rsearch | |
| | | DDBJ databases | Institute, Department of | |
| | | | Human Gene Research; | |
| | | | 1532-3, Yana, | |
| | | | Kisarazu, Chiba 292- | |
| | | | 0812, Japan | |
| sbg1546354LRR | Leucine-rich | SC: AL353746 | Human neuronal | Membrane- |
| | repeat (LRR) | Directly submitted | leucine-rich repeat | bound |
| | protein | (07 SEP. 2000) by | protein, gi: 12309630 | |
| | | Sanger Centre, | Submitted (17 JAN. | |
| | | Hinxton, | 2001) by Sanger | |
| | | Cambridgeshire, | Centre, Hinxton, | |
| | | CB10 1SA, UK | Cambridgeshire, CB10 | |
| | | | 1SA, UK. | |
| sbg1555869CABP | S-100 CABP | GB: AC005921 | Human S100 calcium- | Cytosolic |
| | | Direct submitted | binding protein A10, | |
| | | (03 NOV. 1998) | gi: 4506761 | |
| | | Whitehead | Kube, E., Weber, K. and | |
| | | Institute/MIT Center | Gerke, V. 1991. Gene | |
| | | for Genome | 102:255-259. | |
| | | Research, 320 | | |
| | | Charles Street, | | |
| | | Cambridge, MA | | |
| | | 02141, USA | | |
| sbg1518024NKR | NK-receptor | GB: AL133414 | Human natural killer | Membrane- |
| | | Direct submitted (23 | cell inhibitory receptor | bound |
| | | MAR. 2000) Sanger | KIR2DL3, gi: 2760897 | |
| | | Centre, Hinxton, | Direct submitted (02 | |
| | | Cambridgeshire, | SEP. 1997) Structural | |
| | | CB10 1SA, UK | Biology, Stanford | |
| | | | University, Sherman | |
| | | | Fairchild Building, | |
| | | | Stanford University | |
| | | | School of Medicine, | |
| | | | Stanford, CA 94305- | |
| | | | 5400, USA. | |
| sbg1525809WNT8 | WNT-8 | GB: AC004826 | Human WNT8A, | Secreted |
| | protein | Direct submitted | gi: 14495176 | |
| | | (07 OCT. 2000) | Saitoh, T. and Katoh, M. | |
| | | Department of | Int. J. Oncol. 19 (1), | |
| | | Genetics, | 123-127 (2001) | |
| | | Washington | | |
| | | University, 4444 | | |
| | | Forest Park Avenue, | | |
| | | St. Louis, Missouri | | |
| | | 63108, USA. | | |
| sbg1519904cdk | Cyclin- | EMBL: AL359916 | Human cell division | Cytosolic |
| | dependent | Directly submitted | protein kinase4 | |
| | kinase | (29 SEP. 2000) by | (CDK4), gi: 12830335 | |
| | | Sanger Centre, | Directly submitted (08 | |
| | | Hinxton, | FEB. 2001) by Sanger | |
| | | Cambridgeshire, | Centre, Hinxton, | |
| | | CB10 1SA, UK | Cambridgeshire, CB10 | |
| | | | 1SA, UK | |
| sbg1521449connexin | Connexin | EMBL: AC004977 | Human transport | Membrane- |
| | protein | Submitted (09 | protein TPPT-31, | bound |
| | | MAY 2001) | geneseqp: B60111 | |
| | | Department of | Filed by (INCY-) | |
| | | Genetics, | INCYTE GENOMICS | |
| | | Washington | INC | |
| | | University, 4444 | Publication number and | |
| | | Forest Park Avenue, | filing date: | |
| | | St. Louis, Missouri | WO200078953-A2, | |
| | | 63108, USA | 28 DEC. 2000 | |
| sbg1522659Siglec | Sialic acid- | GB: AC011452 | Human sialic acid- | Membrane- |
| | binding Ig- | Directly submitted | binding lectin 11, gi: | bound |
| | like lectin | (07 OCT. 1999) by | 14718451 | |
| | protein | DOE Joint Genome | Submitted (17 JAN. | |
| | | Institute, 2800 | 2001) Glycobiology | |
| | | Mitchell Drive, | Research and Training | |
| | | Walnut Creek, | Center, University of | |
| | | CA 94598, USA | California, San Diego, | |
| | | | 9500 Gilman Drive, La | |
| | | | Jolla, CA 92093-0687, | |
| | | | USA | |
| sbg1528389ITI | Kunitz-type | SC: AL050348 | Human novel protein | Secreted |
| | protease | Directly submitted | with Kunitz/Bovine | |
| | inhibitor | (27 NOV. 2000) by | pancreatic trypsin | |
| | | Sanger Centre, | inhibitor domain, | |
| | | Hinxton, | gi: 13559983, | |
| | | Cambridgeshire, | Submitted (04 APR. | |
| | | CB10 1SA, UK | 2001) by Sanger | |
| | | | Centre, Hinxton, | |
| | | | Cambridgeshire, CB10 | |
| | | | 1SA, UK. | |
| sbg1526964ISLR | Immunoglobulin | GB: AP001065 | Human leucine-rich | Secreted |
| | superfamily | Submitted (12 JAN. | repeat-containing 3, gi: | |
| | containing | 2000) to the | 13562088 | |
| | leucine-rich | DDBJ/EMBL/GenBank | Submitted (27 MAR. | |
| | repeat | databases by | 2001) by Yoonsoo | |
| | | Nobuyoshi Shimizu, | Hahn, Korea Research | |
| | | Keio University, | Institute of Bioscience | |
| | | School of Medicine, | & Biotechnology, | |
| | | Molecular Biology; | Genome Research | |
| | | 35 Shinanomachi, | Center; 52 Eoeun-dong, | |
| | | Shinjuku-ku, Tokyo | Yuseong-gu, Taejon | |
| | | 160-8582, Japan | 305-333, South Korea | |
| sbg1539439TSPK | Ser/Thr | EMBL: AC008476 | Human serine/threonine | Cytosolic |
| | protein kinase | Directly submitted | kinase FKSGS1, gi: | |
| | | (03 AUG. 1999) by | 14042947 | |
| | | Production | Submitted (07 FEB. | |
| | | Sequencing Facility, | 2001) Beijing | |
| | | DOE Joint Genome | Fengkesheng Function | |
| | | Institute, 2800 | Gene Technology Ltd., | |
| | | Mitchell Drive, | 4 Tou Tiao Lu Chang | |
| | | Walnut Creek, CA | Street, Xuanwu | |
| | | 94598, USA. | District, Beijing | |
| | | | 100050, P. R. China | |
| sbg1571349lipase | Esterase and | EMBL: AC020549 | Human membrane | Membrane- |
| | phospholipase | Directly submitted | associated protein | bound |
| | | (08 NOV. 2000) by | MEMAP-15. | |
| | | Department of | geneseqp: B74709 | |
| | | Genetics, | Filed by (INCY-) | |
| | | Washington | INCYTE GENOMICS | |
| | | University, 4444 | INC | |
| | | Forest Park Avenue, | Publication number and | |
| | | St. Louis, Missouri | filing date: | |
| | | 63108, USA | WO200112662-A2, | |
| | | | 22 FEB. 2001 | |
| sbg1565539aCONNEXIN | Connexin | SC: AL121749 | Human FZD8 gene | Membrane- |
| | | Directly submitted | product (frizzled | bound |
| | | (27 APR. 2000) by | (Drosophila) homolog | |
| | | Sanger Centre, | 8), gi: 10334641 | |
| | | Hinxton, | Directly submitted (27 | |
| | | Cambridgeshire, | APR. 2000) by Sanger | |
| | | CB10 1SA, UK | Centre, Hinxton, | |
| | | | Cambridgeshire, CB10 | |
| | | | 1SA, UK | |
| sbg1565539bCONNEXIN | Connexin | SC: AL121749 | Human FZD8 gene | Membrane- |
| | | Directly submitted | product (frizzled | bound |
| | | (27 APR. 2000) by | (Drosophila) homolog | |
| | | Sanger Centre, | 8), gi: 10334641 | |
| | | Hinxton, | Directly submitted (27 | |
| | | Cambridgeshire, | APR. 2000) by Sanger | |
| | | CB10 1SA, UK | Centre, Hinxton, | |
| | | | Cambridgeshire, CB10 | |
| | | | 1SA, UK | |
| sbg1563294acadherin | Cadherin | SC: AL109928 | Human cadherin-like | Secreted |
| | | Submitted (28 | protein VR20, isoform | |
| | | MAR. 2001) Sanger | 1, gi: 7981305 | |
| | | Centre, Hinxton, | Submitted (28 MAR. | |
| | | Cambridgeshire, | 2001) Sanger Centre, | |
| | | CB10 1SA, UK. | Hinxton, | |
| | | | Cambridgeshire, CB10 | |
| | | | 1SA, UK. | |
| sbg1563294bcadherin | Cadherin | SC: AL109928 | Human cadherin-like | Secreted |
| | | Submitted (28 | protein VR20, isoform | |
| | | MAR. 2001) Sanger | 3, gi: 12743872 | |
| | | Centre, Hinxton, | Submitted (28 MAR. | |
| | | Cambridgeshire, | 2001) Sanger Centre, | |
| | | CB10 1SA, UK. | Hinxton, | |
| | | | Cambridgeshire, CB10 | |
| | | | 1SA, UK. | |
| sbg1563509ECM | Hemicentin | SC: AL133553, | Human hemicentin, | Secreted |
| | | SC: AL135796, | gi: 14575679 | |
| | | SC: AL121996, | Submitted (02 JUN. | |
| | | EMBL: AL135797, | 1999) by National | |
| | | GB: AL391827 | Human Genome | |
| | | Submitted by | Research Institute, | |
| | | Sanger Centre, | National Institutes of | |
| | | Hinxton, | Health, 49 Convent | |
| | | Cambridgeshire, | Drive, Building 49 | |
| | | CB10 1SA, UK on | Room 4A22, Bethesda, | |
| | | 26 JUL. 2000, 18 | MD 20892, USA | |
| | | JUL. 2000, 15 | | |
| | | MAY 2001, 31 | | |
| | | OCT. 2000 and 09 | | |
| | | MAY 2001 | | |
| | | respectively. | | |
| | | SC: AL118582, | | |
| | | Ressourcenzentrum, | | |
| | | Heubnerweg 6, | | |
| | | 14059 Berlin- | | |
| | | Charlottenburg, | | |
| | | GERMANY | | |
| sbg1552159Serprotease | Serine | EMBL: AC012151 | Xenopus laevis | Membrane- |
| | protease | submitted (19 SEP. | polyprotein, gi: | bound |
| | | 2000) by Human | 7512178 | |
| | | Genome Sequencing | Submitted (?? MAR. | |
| | | Center, Department | 1998) to the EMBL | |
| | | of Molecular and | Data Library by Yang, | |
| | | Human Genetics, | J. C., Lindsay, L. L. and | |
| | | Baylor College of | Hedrick, J. L. | |
| | | Medicine, One | | |
| | | Baylor Plaza, | | |
| | | Houston, TX 77030. | | |
| | | USA | | |
| sbg1551159thymosinb4 | Thymosin | GB: AC006312 | Mouse thymosin beta 4, | Secreted |
| | beta4 | Submitted (13 APR. | gi: 10946578 | |
| | | 1999) by Whitehead | Rudin CM, Engler P, | |
| | | Institute/MIT Center | Storb U. J Immunol | |
| | | for Genome | 1990 Jun. | |
| | | Research, 320 | 15; 144(12):4857-62 | |
| | | Charles Street, | | |
| | | Cambridge, MA | | |
| | | 02141, USA | | |
| SBH1686954.SOCS | Cytokine | GS: R-661F4 found | Mouse SOCS14 protein | Cytosolic |
| | signalling | at Genoscope | geneseqp: W62625 | |
| | protein | | Filed by (HALL-) | |
| | | | HALL INST | |
| | | | MEDICAL RES | |
| | | | WALTER & ELIZA | |
| | | | Publication number and | |
| | | | filing date: | |
| | | | WO9820023-A1, | |
| | | | 14 MAY 1998 | |
| sbg1548844slit | Slit | GB: AC004152 | Human membrane | Membrane- |
| | | Submitted (18 FEB. | associated protein | bound |
| | | 1998) by Joint | MEMAP-11. | |
| | | Genome Institute, | geneseqp: B74705 | |
| | | Lawrence Livermore | Filed by (INCY-) | |
| | | National Laboratory, | Incyte Genomics Inc. | |
| | | 7000 East Ave., | Publication number and | |
| | | Livermore, CA | filing date: | |
| | | 94551, USA | WO200112662-A2 | |
| | | | 22 FEB. 2001 | |
| sbg1545249GGT | Gamma- | EMBL: AC023491 | Human glutamyl | Membrane- |
| | glutamyltrans- | found at European | transpeptidase, | bound |
| | peptidase | Molecular Biology | gi: 4885271 | |
| | | Laboratory | Hum. Genet. 76 (3), | |
| | | | 283-286 (1987). | |
| SBh1813899.Y82017 | Ankyrin | GB: AL512624 | Human immunogenic | Cytosolic |
| | motif protein | Submitted (12 FEB. | prostate tumour protein, | |
| | | 2001) by | geneseqp: Y82017 | |
| | | Genoscope - Centre | Filed by (CORI-) | |
| | | National de | CORIXA CORP | |
| | | Sequencage: BP | Publication number and | |
| | | 191 91006 EVRY | filing date: | |
| | | cedex - FRANCE | WO200004149-A2, | |
| | | | 27 JAN. 2000 | |
| sbg1536324mapkkk | Mitogen- | SC: AL133380 | Human mitogen- | Possibly |
| | activated | found at Sanger | activated protein kinase | cytosolic |
| | protein kinase | Centre | kinase kinase 10, | |
| | | | gi: 4505263 | |
| | | | Eur. J. Biochem. 213 | |
| | | | (2), 701-710 (1993) | |
| gsk1810944BrCaAg | Ankyrin | GB: AC060814 | Human immunogenic | Possibly |
| | repeat/breast | Submitted (20 APR. | prostate tumour protein, | secreted |
| | cancer | 2000) Whitehead | geneseqp: Y82017 | |
| | antigen | Institute/MIT Center | Filed by (CORI-) | |
| | | for Genome | CORIXA CORP | |
| | | Research, 320 | Publication number and | |
| | | Charles Street, | filing date: | |
| | | Cambridge, MA | WO200004149-A2, | |
| | | 02141, USA | 27 JAN. 2000 | |
| sbg1535914AIP | Inhibitor of | GB: AC004223 | Mouse putative protein, | Secreted |
| | apoptosis | Submitted (25 FEB. | gi: 12840594 | |
| | protein | 1998) by Whitehead | Methods in | |
| | | Institute/MIT Center | enzymology. 303, 19- | |
| | | for Genome | 44 (1999) | |
| | | Research, 320 | | |
| | | Charles Street, | | |
| | | Cambridge, MA | | |
| | | 02141. | | |
| sbg1530894aCE | Carboxylesterase/ | EMBL: AC007335 | Macaca fascicularis | Secreted |
| | lipase | Submitted (03 | hypothetical protein, | |
| | | NOV. 2000) by | gi: 13874532 | |
| | | Production | Submitted (27 APR. | |
| | | Sequencing Facility, | 2001) by Katsuyuki | |
| | | DOE Joint Genome | Hashimoto, National | |
| | | Institute, 2800 | Institute of Infectious | |
| | | Mitchell Drive, | Diseases, Division of | |
| | | Walnut Creek, CA | Genetic Resources; 23- | |
| | | 94598, USA | 1, Toyama 1-chome, | |
| | | | Shinjuku-ku, Tokyo | |
| | | | 162-8640, Japan | |
| sbg1530894bCE | Carboxylesterase/ | EMBL: AC007335 | Macaca fascicularis | Cytosolic |
| | lipase | Submitted (03 | hypothetical protein, | |
| | | NOV. 2000) by | gi: 13874532 | |
| | | Production | Submitted (27 APR. | |
| | | Sequencing Facility, | 2001) by Katsuyuki | |
| | | DOE Joint Genome | Hashimoto, National | |
| | | Institute, 2800 | Institute of Infectious | |
| | | Mitchell Drive, | Diseases, Division of | |
| | | Walnut Creek, CA | Genetic Resources; 23- | |
| | | 94598, USA | 1, Toyama 1-chome, | |
| | | | Shinjuku-ku, Tokyo | |
| | | | 162-8640, Japan | |
| gsk.1813134.NaI | Sodium | GB: AC063951 | Human solute carrier | Cytosolic |
| | iodide | Submitted (22 APR. | family 5 (sodium iodide | |
| | symporter | 2000) by Human | symporter), | |
| | | Genome Sequencing | gi: 4507035 | |
| | | Center, Department | Biochem. Biophys. Res. | |
| | | of Molecular and | Commun. 226 (2), 339- | |
| | | Human Genetics, | 345 (1996) | |
| | | Baylor College of | | |
| | | Medicine, One | | |
| | | Baylor Plaza, | | |
| | | Houston, TX 77030, | | |
| | | USA | | |
| gsk1811484BrCaAg | Ankyrin | GB: AC013269 | Human immunogenic | Cytosolic |
| | repeat/breat | Submitted (05 | prostate tumour protein, | |
| | cancer | NOV. 1999) | geneseqp: Y82017 | |
| | antigen | Genome Sequencing | Filed by (CORI-) | |
| | | Center, Washington | CORIXA CORP | |
| | | University School of | Publication number and | |
| | | Medicine, 4444 | filing date: | |
| | | Forest Park | WO200004149-A2, | |
| | | Parkway, St. Louis, | 27 JAN. 2000 | |
| | | MO 63108, USA | | |
| sbg1529984aSproteinase | Serine | GB: AC005570 | Human plasminogen- | Secreted |
| | proteinase | Submitted (01 SEP. | like protein, geneseqp: | |
| | | 1998) by Center for | AAE00290 | |
| | | Human Genome | Filed by (HUMA-) | |
| | | Studies, DOE Joint | HUMAN GENOME | |
| | | Genome Institute, | SCI INC | |
| | | Los Alamos National | Publication number and | |
| | | Laboratory, MS | filing date: | |
| | | M888, Los Almos, | WO200124815-A1 | |
| | | NM 87545, USA | 12 APR. 2001 | |
| sbg1529984bSproteinase | Serine | GB: AC005570 | Human plasminogen- | Secreted |
| | proteinase | Submitted (01 SEP. | like protein, geneseqp: | |
| | | 1998) by Center for | AAE00290 | |
| | | Human Genome | Filed by (HUMA-) | |
| | | Studies, DOE Joint | HUMAN GENOME | |
| | | Genome Institute, | SCI INC | |
| | | Los Alamos National | Publication number and | |
| | | Laboratory, MS | filing | |
| | | M888, Los Almos, | date: WO200124815-A1 | |
| | | NM 87545, USA | 12 APR. 2001 | |
| SBGKIN113 | G protein- | GB: AC068693 | Squirrel cone opsin | Cytosolic |
| | coupled | Submitted (06 | kinase GRK7, | |
| | receptor | MAY 2000) by | gi: 4001826 | |
| | kinase | Whitehead | Weiss E R, Raman D, | |
| | | Institute/MIT Center | Shirakawa S, Ducceschi | |
| | | for Genome | M H, Bertram P T, | |
| | | Research, 320 | Wong F, Kraft T W, | |
| | | Charles Street, | Osawa S. | |
| | | Cambridge, MA | Mol Vis 1998 Dec. | |
| | | 02141, USA | 8; 4:27 | |
| gsk305961GDNa | Serpin | GB: AL137780 | Human glia-derived | Secreted |
| | | Sanger Centre, | nexin I alpha precursor, | |
| | | Hinxton, | gi: 87514 | |
| | | Cambridgeshire, | Biochemistry. 26 (20), | |
| | | CB10 1SA, UK | 6407-6410 (1987) | |
| gsk2402719_2395124BIG2 | Neural cell | AC066608, | Rat neural cell adhesion | Secreted |
| | adhesion | AC087084, | protein BIG-2 | |
| | molecule | AC087094 (Clones: | precursor, gi: 1016012 | |
| | | RP11-785A7, RP11- | Yoshihara, Y., | |
| | | 198p17, 94m9) | Kawasaki, M., | |
| | | Human Genomic | Tamada, A., Nagata, S., | |
| | | Center, Institute of | Kagamiyama, H. and | |
| | | Genetics, Chinese | Mori. K. J. Neurobiol. | |
| | | Academy of | 28 (1), 51-69 (1995) | |
| | | Sciences, Datun | | |
| | | Road, Beijing, | | |
| | | Beijing 100101, | | |
| | | P.R. China | | |
| sbg18525LRR b | Leucine-rich | GB: AC016030 | Macaca fascicularis | Membrane- |
| | repeat (LLR) | Direct submitted | hypothetical protein, | bound |
| | | (19 NOV. 1999) by | gi: 13358942 | |
| | | Whitehead | Submitted (24 FEB. | |
| | | Institute/MIT Center | 2001) by Katsuyuki | |
| | | for Genome | Hashimoto, National | |
| | | Research, 320 | Institute of Infectious | |
| | | Charles Street, | Diseases, Division of | |
| | | Cambridge, MA | Genetic Resources; 23- | |
| | | 02141, USA | 1, Toyama 1-chome, | |
| | | | Shinjuku-ku, Tokyo | |
| | | | 162-8640, Japan | |
| SBhACRP30c | Complement | GB: AC007016 | Human complement- | secreted |
| | C1q/TNF | Submitted (08 May | c1q tumor necrosis | |
| | | 1999) by Department | factor-related protein, | |
| | | of Genetics, | gi: 14734515 | |
| | | Stanford Human | Submitted (20 DEC. | |
| | | Genome Center, 855 | 2000) by | |
| | | Miranda Avenue, | Bioinformatics, | |
| | | Palo, CA 94304 | ZymoGenetics, Inc., | |
| | | | 1201 Eastlake Ave. | |
| | | | East, Seattle, WA | |
| | | | 98102, USA | |
| sbg123493SLITc | Slit-like | SC: AL157714 | Macaca fascicularis | Membrane- |
| | protein | Submitted (20 JAN. | hypothetical protein, | bound |
| | | 2001) by Sanger | gi: 15208206 | |
| | | Centre, Hinxton, | Submitted (14 AUG. | |
| | | Cambridgeshire, | 2001) by Katsuyuki | |
| | | CB10 1SA, UK. | Hashimoto, National | |
| | | | Institute of Infectious | |
| | | | Diseases, Division of | |
| | | | Genetic Resources; | |
| | | | 23-1, Toyama 1- | |
| | | | chome, Shinjuku-ku, | |
| | | | Tokyo 162-8640, | |
| | | | Japan | |
| sbg102200MCTc | Monocarb | GB: AC015918 | Mouse riken cDNA | Membrane- |
| | oxylate | Directly submitted | ORF, AK005699 | bound |
| | cotransporter | (17 NOV. 1999) | The RIKEN Genome | |
| | (MCT1) | by Whitehead | Exploration Research | |
| | | Institute/MIT | Group Phase II Team | |
| | | Center for Genome | and FANTOM | |
| | | Research, 320 | Consortium. | |
| | | Charles Street, | Nature 409, 685-690 | |
| | | Cambridge, MA | (2001) | |
| | | 02141, USA. | | |

TABLE III

| | | Associated |
|---|---|---|
| Gene Name | Uses | Diseases |
| sbg1571549cystatin-re | An embodiment of the invention | Cancer and wound |
| | is the use of sbg1571549cystatin-re | healing disorders |
| | in bone resorption and in a variety | |
| | of pathological conditions, including | |
| | cancer progression and metastasis, | |
| | stroke, inflammatory diseases, and | |
| | infections. A close homologue of | |
| | sbg1571549cystatin-re is a cystatin- | |
| | related protein, known as testatin, | |
| | a family of small secreted protease | |
| | inhibitors. Cystatins and their target | |
| | proteases are likely to play important | |
| | roles in normal processes such as bone | |
| | resorption (Lerner, U. H., Johansson, | |
| | L., Ranjso, M., Rosenquist, J. B., | |
| | Reinholt, F. P. & Grubb, A. 1997. Acta | |
| | Physiol. Scand. 161, 8192) and in a | |
| | variety of pathological conditions, | |
| | including cancer progression and meta- | |
| | stasis, stroke, inflammatory diseases, | |
| | and infections (Henskens, Y. M., | |
| | Veerman, E. C. & Nieuw Amerongen, A. V. | |
| | 1996. Biol. Chem. Hoppe-Seyler 377, | |
| | 7186). | |
| sbg1558434SRCR | An embodiment of the invention is the | Atherosclerosis |
| | use of sbg1558434SRCR in atherogenesis. | |
| | Close homologues of sbg1558434SRCR are | |
| | scavenger receptor cysteine-rich | |
| | domains. The scavenger receptors were | |
| | detected in the macrophages of lipid- | |
| | rich atherosclerotic lesions | |
| | (Matsumoto A, Naito M, Itakura H, | |
| | Ikemoto S, Asaoka H, Hayakawa I, | |
| | Kanamori H, Aburatani H, Takaku F, | |
| | Suzuki H, et al. Proc Natl Acad Sci. | |
| | USA 1990 Dec.; 87(23):9133-7). | |
| | Recently, Gough et al. also demonstrated | |
| | that in human and rabbit atherosclerotic | |
| | lesions, the scavenger receptor class A | |
| | was mainly expressed by macrophage- | |
| | derived foam cells, whereas only a low | |
| | level of expression was found in the | |
| | aortic endothelium (Gough, P. J., Greaves, | |
| | D. R., Suzuki, H., Hakkinen, T., Hiltunen, | |
| | M. O., Turunen, M., Ylä Herttuala, | |
| | S., Kodama, T., Gordon, S. 1999. | |
| | Arterioscler. Thromb. Vasc. Biol. | |
| | 19:461-471). | |
| sbg1546354LRR | An embodiment of the invention is the | Wound healing, |
| | use of sbg1546354LRR in wound healing | Parkinson's |
| | and tissue repair and in neural develop- | disease, and |
| | ment and the adult nervous system dys- | Alzheimer's |
| | functions including Parkinson's disease | disease |
| | and Alzheimer's disease. Close homologues | |
| | of sbg1546354LRR are leucine-rich repeat | |
| | (LRR) proteins such as connectin, slit, | |
| | chaoptin, and Toll. Mouse brain cDNAs | |
| | which encode three new independent LRR | |
| | proteins (neuronal leucine-rich repeat: | |
| | NLRR-1, NLRR-2, NLRR-3) were reported | |
| | (Taguchi A, Wanaka A, Mori T, Matsumoto | |
| | K, Imai Y, Tagaki T, Tohyama M. 1996. | |
| | Brain Res Mol Brain Res Jan.; 35(1-2): | |
| | 31-40, Taniguchi H, Tohyama M, Takagi | |
| | T. 1996 Brain Res Mol Brain Res Feb.; | |
| | 36(1):45-52). Both NLRR-1 and NLRR-2 | |
| | mRNAs were expressed primarily in the | |
| | central nervous system (CNS); NLRR-1 | |
| | mRNA was also detected in the non- | |
| | neuronal tissues such as cartilage, | |
| | while NLRR-2 mRNA expression was con- | |
| | fined to the CNS at all developmental | |
| | stages. NLRR-3 mRNA increased in | |
| | layers 2-3 in the injured cerebral | |
| | cortex (Ishii N, Wanaka A, Tohyama M. | |
| | 1996. Brain Res Mol Brain Res Aug.; | |
| | 40(1):148-52). These results suggest | |
| | that NLRR-1 and NLRR-2 may play sig- | |
| | nificant but distinct roles in neural | |
| | development and the adult nervous | |
| | system. While NLRR-3 may be an | |
| | important component of the patho- | |
| | physiological response to brain | |
| | injury. | |
| sbg1555869CABP | An embodiment of the invention is the | Cancer, infection, |
| | use of sbg1555869CABP in cell growth | autoimmune disorder, |
| | and differentiation, cell cycle | hematopoietic |
| | regulation and metabolic control. Close | disorder, wound |
| | homologues of sbg1555869CABP are S-100, | healing disorders, |
| | a member of the calcium and zinc- | inflammation, |
| | binding protein family. Calcium ions | Alzheimer's disease, |
| | act as a second messenger to interact | neoplastic diseases, |
| | with calcium binding proteins, and thus | nervous system |
| | control biological processes. Many | related disorders, and |
| | diseases, including Alzheimer's disease | cardiovascular |
| | and neoplastic diseases, are associated | disorders |
| | with abnormal calcium ion levels | |
| | (Schafer B W, Heizmann C W. 1996. Trends | |
| | Biochem Sci 21:134-40). Therefore, | |
| | intracellular calcium ion levels and | |
| | calcium ion signaling must be tightly | |
| | regulated. For example, S100 A10, serves | |
| | as a ligand of the tyrosine kinase | |
| | substrate, annexin II (Kube E, Weber K, | |
| | Gerke V. 1991. Gene 102:255-9). | |
| | Evidence support S-100 involvement in | |
| | cell growth and differentiation, cell | |
| | cycle regulation and metabolic control | |
| | (Lackmann M., Cornish C. J., Simpson | |
| | R. J., Moritz R. L., Geczy C. L. 1992. | |
| | J. Biol. Chem. 267:7499-7504). | |
| sbg1518024NKR | An embodiment of the invention is the | Cancer, infection, and |
| | use of sbg1518024NKR in the recognition | autoimmune disorder |
| | of target cells by natural killer (herein | |
| | “NK”) cells and subsequent cell lysis. | |
| | Close homologues of sbg1518024NKR are a | |
| | family of cell surface molecules, the | |
| | receptors of NK cells. This lysis is a | |
| | defense mechanism against pathogens and | |
| | tumor cells that downregulate MHC | |
| | expression to avoid T-cell immune response. | |
| | Normal cells can escape lysis because | |
| | their MHC molecules are recognized by | |
| | NK-cell inhibitory receptors (Fan Q R, | |
| | Mosyak L, Winter C C, Wagtmann N, Long | |
| | E O, Wiley D C. 1997. Nature 389:96-100). | |
| sbg1525809WNT8 | An embodiment of the invention is the | Cancer, infection, |
| | use of sbg1525809WNT8 in the regulation | autoimmune disorder, |
| | of axis formation and hindbrain | hematopoietic |
| | patterning. Close homologues of | disorder, wound |
| | sbg1525809WNT8 include mouse and | healing disorders, and |
| | chicken WNT-8. WNT-8 is only expressed | inflammation |
| | during embryogenesis (Bouillet P, | |
| | Oulad-Abdelghani M, Ward S J, Bronner | |
| | S, Chambon P, Dolle P. 1996 Mech Dev | |
| | 58:141-52, Hume C R, Dodd J. 1993 | |
| | Development 119:1147-60). | |
| sbg1519904cdk | An embodiment of the invention is the | Cancer |
| | use of sbg1519904cdk, an important | |
| | component in cell cycle control, as a | |
| | potential target for novel anticancer | |
| | drug discovery. Close homologues of | |
| | sbg1519904cdk are Cdk4 and its close | |
| | relative Cdk6. In mammalian cells, | |
| | there are two classes of Cdks that | |
| | function at the G1/S phase transition | |
| | (Ekholm S V, Reed S I, Curr Opin Cell | |
| | Biol 2000 Dec.; 12(6):676-84). | |
| | Cdk4 and Cdk6 are driven by three D- | |
| | type cyclins: D1, D2 and D3. The | |
| | primary target of these activities | |
| | is pRb and related protiens (Grana X, | |
| | Garriga J, Mayol X. Oncogene 1998, | |
| | 17:3365-3383). The interaction of the | |
| | pRb-E2F complexes with cell-cycle | |
| | control genes has been shown to be | |
| | important for pRb-mediated cell- | |
| | cycle arrest (Zhang, P., Wong, C., | |
| | Liu, D., Finegold, M., Harper, J. | |
| | W. and Elledge, S. J., 1999. Genes | |
| | Dev. 13, pp. 213-224.). The E2F | |
| | target genes induced as a consequence | |
| | of the disruption of pRb-E2F complexes | |
| | include some of the enzymes involved | |
| | in DNA metabolism, some protooncogenes, | |
| | and some cell-cycle regulatory pro- | |
| | teins (Dyson, N. 1998. Genes Dev. 12, | |
| | pp. 2245-2262). Cdk4 is also | |
| | reported mis-regulated in 60-70% of | |
| | human cancers (Soni R, Muller L, Furet | |
| | P, Schoepfer J, Stephan C, Zumstein- | |
| | Mecker S, Fretz H, Chaudhuri B. | |
| | Biochem Biophys Res Commun 2000 Sep. | |
| | 7; 275(3):877-84). | |
| sbg1521449connexin | An embodiment of the invention is | Cardiovascular |
| | the use of sbg1521449connexin in | disease, osteogenesis, |
| | the propagation of the action | and wound healing |
| | potential from the conduction | disorders |
| | system to the working myocardium. | |
| | Close homologues of | |
| | sbg1521449connexin include connexin | |
| | 43 and connexin 45. Two members of | |
| | the connexin family of gap junction | |
| | proteins, connexin 43 (Cx43; Gj alpha | |
| | 1) and connexin 45 (Cx45; Gj alpha | |
| | 6) have been associated with osteo- | |
| | genesis (Civitelli R, Beyer E C, | |
| | Warlow P M, Robertson A J, Geist S | |
| | T, Steinberg T H. 1993. J Clin | |
| | Invest 91:1888-1896). Human | |
| | connexin 45 was cloned and, like | |
| | Cx43, is expressed throughout the | |
| | heart. Expression of Cx45 in heart | |
| | is regional and associated with | |
| | the atrioventricular node, the | |
| | bundle and peripheral ventricular | |
| | conduction system where it is | |
| | located at the borderzone between | |
| | the purkinje-fibers and the working | |
| | ventricular myocardium. In this | |
| | position, Cx45 may play a signifi- | |
| | cant role in the propagation of | |
| | the action potential from the con- | |
| | duction system to the working | |
| | myocardium (Kanter H L, Saffitz | |
| | J E, Beyer E C. J Mol Cell Cardiol | |
| | 1994 Jul.; 26(7):861-8. S. R. | |
| | Coppen, E. Dupont, S. Rothery and | |
| | N. J. Severs. Circ. Res. 82 | |
| | (1998), pp. 232243). Cx45-deficient | |
| | mouse embryos exhibited striking | |
| | abnormalities in vascular development | |
| | and died between embryonic day (E) | |
| | 9.5 and 10.5 (Kruger O, Plum A, Kim | |
| | J, Winterhager E, Maxeiner S, | |
| | Hallas G, Kirchhoff S, Traub O, | |
| | Lamers W H, Willecke K. Development | |
| | 2000 Oct.; 127(19):4179-93). | |
| sbg1522659Siglec | An embodiment of the invention is | Cancer, infection, |
| | the use of sbg1522659Siglec to help | autoimmune disorder, |
| | prevent undesirable self- | hematopoietic |
| | reactivity and tissue damage and | disorder, wound |
| | permit effective killing of non- | healing disorders, and |
| | sialylated pathogens. Close homo- | inflammation diseases |
| | logues of sbg1522659Siglec are | |
| | the five proteins Siglec-5 to | |
| | Siglec-9. Despite their sequence | |
| | similarity, all novel Siglecs | |
| | characterized to date are expressed | |
| | on distinct subsets of hemopoietic | |
| | cells, such as neutrophils (Siglec- | |
| | 5), B-cells (Siglec-6), natural | |
| | killer (NK) cells (Siglec7), eosino- | |
| | phils (Siglec-8) and neutrophils and | |
| | monocytes (Siglec-9). Each of these | |
| | Siglecs also exhibits distinct carbo- | |
| | hydrate binding properties (Zhang | |
| | J Q, Nicoll G, Jones C, Crocker P R. | |
| | J Biol Chem 2000 Jul. 21; 275(29): | |
| | 22121-6, Crocker, P. R., Clark, | |
| | E. A., Filbin, M., Gordon, S., Jones, | |
| | Y., Kehrl, J. H., Kelm, S., Le | |
| | Douarin, N., Powell, L., Roder, J., | |
| | Schnaar, R. L., Sgroi, D. C., | |
| | Stamenkovic, K., Schauer, R., | |
| | Schachner, M., van den Berg, T. K., | |
| | van der Merwe, P. A., Watt, S. M., | |
| | and Varki, A. (1998) Glycobiology 8, | |
| | v). CD22 (Siglec-6) was shown to | |
| | downregulate the B cell receptor- | |
| | mediated cell triggering. In | |
| | addition, cross-linking of Siglec-7 | |
| | in human NK cells inhibited signal | |
| | transduction of spontaneous NK cell- | |
| | mediated cytotoxicity (Falco, M., | |
| | Biassoni, R., Bottino, C., Vitale, | |
| | M., Sivori, S., Augugliaro, R., | |
| | Moretta, L., and Moretta, A. (1999) | |
| | J. Exp. Med. 190, 793-802). It | |
| | is possible that the immune receptor | |
| | tyrosine-based inhibitory motifs | |
| | (ITIM)-containing Siglecs mediate | |
| | negative regulation of cellular | |
| | activation events via sialic acid | |
| | recognition. | |
| sbg1528389ITI | An embodiment of the invention is | Cancer, pancreatitis, |
| | the use of sbg1528389ITI to | inflammation, |
| | modulate premature activation of | respiratory and wound |
| | pancreatic proteases and decrease | healing disorder |
| | the rate of mucus digestion, | |
| | possibly by lowering the threshold | |
| | for pancreatitis. Close homologues | |
| | of sbg1528389ITI include a pan- | |
| | creatic secretory trypsin inhibitor | |
| | (PSTI). sbg1528389ITI has a function- | |
| | al domain similiar to pancreatic | |
| | trypsin inhibitor and/or neutrophil | |
| | elastase inhibitors. Based on | |
| | genetic-linkage studies, PSTI muta- | |
| | tions are associated with | |
| | pancreatitis and are disease modi- | |
| | fying, possibly by lowering the | |
| | threshold for pancreatitis from | |
| | other genetic or environmental | |
| | factors (Pfutzer R H, Barmada M M, | |
| | Brunskill A P, Finch R, Hart P S, | |
| | Neoptolemos J, Furey W F, Whitcomb | |
| | D C. Gastroenterology 2000 Sep.; | |
| | 119(3):615-23). PSTI was also | |
| | shownd to prevent premature acti- | |
| | vation of pancreatic proteases and | |
| | to decrease the rate of mucus | |
| | digestion by luminal proteases within | |
| | the stomach and colon. In addition, | |
| | it was suggested that PSTI may be | |
| | involved in both the early and late | |
| | phases of the healing response follow- | |
| | ing injury by increasing the proli- | |
| | feration of a variety of cell lines | |
| | and stimulating cell migration | |
| | (Marchbank T, Freeman T C, Playford | |
| | R J. Digestion 1998; 59(3):167-74). | |
| | Finally, neutrophil elastase in- | |
| | hibitors and other members of Kunitz- | |
| | type protease inhibitor family are | |
| | suspected of playing an important | |
| | role in the extra-cellular matrix | |
| | biology which is a key part of | |
| | tumoral invasion and chronic broncho- | |
| | pulmonary diseases (Cuvelier A, Muir | |
| | J F, Martin J P, Sesboue R. Rev Mal | |
| | Respir 2000 Apr.; 17(2):437-46). | |
| sbg1526964ISLR | An embodiment of the invention is | Cancer, infection, |
| | the use of sbg1526964ISLR, a | autoimmune disorder, |
| | secreted protein, for the inhibition | hematopoietic |
| | of neoplastic cell growth and | disorder, wound |
| | regulation of the orderly assembly | healing disorders, and |
| | of extracellular matrices. Close | inflammation disease |
| | homologues of sbg1526964ISLR are | |
| | decorin D and slit, members of | |
| | the immunoglobulin superfamily | |
| | containing leucine-rich repeat | |
| | (ISLR) and human PRO221 antitumour | |
| | protein. Decorin has been shown | |
| | to mediate fundamental cellular | |
| | processes, including regulation | |
| | of the orderly assembly of extra- | |
| | cellular matrices, corneal trans- | |
| | parency, tensile strength of skin | |
| | and tendon, viscoelasticity of | |
| | blood vessels, and tumor cell | |
| | proliferation (Kresse, H., Hausser, | |
| | H., and Schönherr, E. (1993) Ex- | |
| | perientia (Basel) 49, 403-416. | |
| | Iozzo, R. V. (1998) Annu. Rev. | |
| | Biochem. 67, 609-652. Iozzo, | |
| | R. V. (1999) J. Biol. Chem. 274, | |
| | 18843-18846). Slit protein | |
| | participates in the formation and | |
| | maintenance of the nervous and | |
| | endocrine systems by protein- | |
| | protein interactions (Itoh A, | |
| | Miyabayashi T, Ohno M, Sakano S. | |
| | Brain Res Mol Brain Res 1998 | |
| | Nov. 20; 62(2):175-86). | |
| sbg1539439TSPK | An embodiment of the invention is | Cancer, infection, |
| | the use of sbg1539439TSPK in | autoimmune disorder, |
| | spermiogenesis, and signaling | hematopoietic |
| | mechanisms including cell pro- | disorder, wound |
| | liferation and transformation. | healing disorders, and |
| | Close homologues of | inflammation disease |
| | sbg1539439TSPK include mouse | |
| | spermiogenesis assosciated | |
| | serine/threonine kinase and | |
| | Ser/Thr kinase Akt/PKB, | |
| | and Ser/Thr kinase CK2. | |
| | Some protein kinases are | |
| | related to carcinogenesis | |
| | and tumor progression (Maemura | |
| | M, Iino Y, Koibuchi Y, Yokoe T, | |
| | and Morishita Y; 1999; Oncology | |
| | 57. Arnaud Besson, Stephen M. | |
| | Robbins and V. Wee Yong. European | |
| | Journal of Biochemistry 263 (3), | |
| | 605-611. Suppl 2:37-44). Ser/Thr | |
| | kinase Akt/PKB has been implicated in | |
| | antiapoptosis and neuronal survival | |
| | (Kauffmann-Zeh A, Rodriguez-Viciana | |
| | P, Ulrich E, Gilbert C, Coffer P, | |
| | Downward J, Evan G. Nature 385, | |
| | 544-548 (1997). Kennedy S G, | |
| | Wagner A J, Conzen S D, Jordan J, | |
| | Bellacosa A, Tsichlis P N, Hay N | |
| | Genes Dev. 11, 701-713 (1997). | |
| | Ikeuchi T, Shimoke K, Kubo T, | |
| | Yamada M, Hatanaka H, Hum Cell 1998 | |
| | Sep.; 11(3):125-40). Another Ser/Thr | |
| | kinase CK2 was shown to be rapid | |
| | modulated in the in nuclear matrix | |
| | response to hormonal and growth | |
| | factor signals and may serve as a | |
| | factor in promoting the transition | |
| | of inactive to active nucleosome | |
| | (Ahmed K. Crit Rev Eukaryot Gene | |
| | Expr 1999; 9(3-4):329-36). | |
| | Another Ser/Thr kinase CK2 was | |
| | shown to be rapid modulated in the | |
| | in nuclear matrix response to hor- | |
| | monal and growth factor signals and | |
| | it may serve as a factor in pro- | |
| | moting the transition of inactive | |
| | to active nucleosome (Ahmed K. Crit | |
| | Rev Eukaryot Gene Expr 1999; | |
| | 9(3-4):329-36). | |
| sbg1571349lipase | An embodiment of the invention is | Cancer, infection, |
| | the use of sbg1571349lipase in | autoimmune disorder, |
| | modulating esterase-mediated | hematopoietic |
| | degradation. Close homologues of | disorder, wound |
| | sbg1571349lipase are rabbit entero- | healing disorders, and |
| | cyte brush border protein and | inflammation diseases |
| | Phospholipases A2. Rabbit entero- | |
| | cyte brush border protein which | |
| | was found to have esterase and | |
| | phospholipase activity (Boll W, | |
| | Schmid-Chanda T, Semenza G, Mantei | |
| | N. J Biol Chem 1993 Jun. 15; | |
| | 268(17):12901-11). Esterase- | |
| | mediated degradation during | |
| | transepithelial transport might | |
| | be a limiting factor for its oral | |
| | absorption, targeting the pro-drug | |
| | to a region of the intestine with | |
| | lower esterase activity could lead | |
| | to an increase in oral absorption | |
| | of the pro-drug (Van Gelder J, | |
| | Shafiee M, De Clercq E, Penninckx | |
| | F, Van den Mooter G, Kinget R, | |
| | Augustijns P. Int J Pharm 2000 Sep. | |
| | 15; 205(1-2):93-100). Phos- | |
| | pholipases A2 has contributed to | |
| | the production of proinflammatory | |
| | lipid mediators, moreover, it | |
| | may be involved in cell responses | |
| | such as apoptosis or tumor cell | |
| | metastatic potential (Chaminade B, | |
| | Le Balle F, Fourcade O, Nauze M, | |
| | Delagebeaudeuf C, Gassama-Diagne A, | |
| | Simon M F, Fauvel J, Chap H. | |
| | Lipids 1999; 34 Suppl: S49-55). | |
| sbg1565539aCONNEXIN | An embodiment of the invention is | Cardiovascular |
| | the use of sbg1565539aCONNEXIN in | diseases, osteogenesis, |
| | the propagation of the action | and wound healing |
| | potential from the conduction system | disorders disease |
| | to the working myocardium. Close | |
| | homologues of sbg1565539aCONNEXIN | |
| | are connexin 43 and connexin 45. | |
| | Two members of the connexin family | |
| | of gap junction proteins, connexin | |
| | 43 (Cx43; Gj alpha 1) and connexin | |
| | 45 (Cx45; Gj alpha 6) have been | |
| | associated with osteogenesis | |
| | (Civitelli R, Beyer E C, Warlow P | |
| | M, Robertson A J, Geist S T, | |
| | Steinberg T H. 1993. J Clin Invest | |
| | 91:1888-1896). Human connexin 45 | |
| | was cloned and, like Cx43, is | |
| | expressed throughout the heart. | |
| | Expression of Cx45 in heart is | |
| | regional and associated with the | |
| | atrioventricular node, the bundle and | |
| | peripheral ventricular conduction | |
| | system where it is located at the | |
| | borderzone between the purkinje- | |
| | fibers and the working ventricular | |
| | myocardium. In this position, Cx45 | |
| | may play a significant role in the | |
| | propagation of the action potential | |
| | from the conduction system to the | |
| | working myocardium (Kanter H L, Saffitz | |
| | J E, Beyer E C. J Mol Cell Cardiol | |
| | 1994 Jul.; 26(7):861-8. S. R. | |
| | Coppen, E. Dupont, S. Rothery and N. | |
| | J. Severs. Circ. Res. 82 (1998), pp. | |
| | 232243). Cx45-deficient mouse embryos | |
| | exhibited striking abnormalities in | |
| | vascular development and died between | |
| | embryonic day (E) 9.5 and 10.5 (Kruger | |
| | O, Plum A, Kirn J, Winterhager E, | |
| | Maxeiner S, Hallas G, Kirchhoff S, | |
| | Traub O, Lamers W H, Willecke K. | |
| | Development 2000 Oct.; 127(19):4179- | |
| | 93). | |
| sbg1565539bCONNEXIN | An embodiment of the invention is the | Cardiovascular |
| | use of sbg1565539bCONNEXIN in the | diseases, osteogenesis, |
| | propagation of the action potential | and wound healing |
| | from the conduction system to the | disorders disease |
| | working myocardium. Close homologues | |
| | of sbg1565539bCONNEXIN are connexin | |
| | 43 and connexin 45. Two members of | |
| | the connexin family of gap junction | |
| | proteins, connexin 43 (Cx43; Gj alpha | |
| | 1) and connexin 45 (Cx45; Gj alpha 6) | |
| | have been associated with osteo- | |
| | genesis (Civitelli R, Beyer E C, | |
| | Warlow P M, Robertson A J, Geist S T, | |
| | Steinberg T H. 1993. J Clin Invest | |
| | 91:1888-1896). Human connexin 45 | |
| | was cloned and, like Cx43, is expressed | |
| | throughout the heart. Expression of | |
| | Cx45 in heart is regional and | |
| | associated with the atrioventricular | |
| | node, the bundle and peripheral | |
| | ventricular conduction system where | |
| | it is located at the borderzone | |
| | between the purkinje-fibers and the | |
| | working ventricular myocardium. In | |
| | this position, Cx45 may play a | |
| | significant role in the propagation | |
| | of the action potential from the | |
| | conduction system to the working | |
| | myocardium (Kanter H L, Saffitz | |
| | J E, Beyer E C. J Mol Cell Cardiol | |
| | 1994 Jul.; 26(7):861-8. S. R. Coppen, | |
| | E. Dupont, S. Rothery and N. J. | |
| | Severs. Circ. Res. 82 (1998), pp. | |
| | 232243). Cx45-deficient mouse | |
| | embryos exhibited striking | |
| | abnormalities in vascular develop- | |
| | ment and died between embryonic | |
| | day (E) 9.5 and 10.5 (Kruger O, | |
| | Plum A, Kirn J, Winterhager E, | |
| | Maxeiner S, Hallas G, Kirchhoff | |
| | S, Traub O, Lamers W H, Willecke | |
| | K. Development 2000 Oct.; 127(19): | |
| | 4179-93). | |
| sbg1563294acadherin | An embodiment of the invention is | Cancer, infection, |
| | the use of sbg1563294acadherin in | autoimmune |
| | epithelial polarization, epithelial | disorder, |
| | sheet or tube fusion, cell | hematopoietic |
| | migration, cell sorting, and axonal | disorder, wound |
| | patterning. A close homologue of | healing disorders and |
| | sbg1563294acadherin is desmocollin, | inflammation |
| | a memeber of the cadherin super- | diseases |
| | family. Antisense expression of a | |
| | desmocollin gene in MDCK cells has | |
| | showed the alteration in desmosome | |
| | plaque assembly (Roberts G A, | |
| | Burdett I D, Pidsley S C, King I A, | |
| | Magee A I, Buxton R S. Eur J Cell | |
| | Biol 1998 Jul.; 76(3):192-203). | |
| | Fat-like cadherins were involved | |
| | in epithelial morphogenesis, | |
| | proliferation control, and epithelial | |
| | planar polarization (Tepass U. Curr | |
| | Opin Cell Biol 1999 Oct.; 11(5): | |
| | 540-8, Takeshi Yagi and Takeichi. | |
| | Genes & Dev 2000. 14(10) 1169-1180). | |
| | Moreover, cadherin has been implicated | |
| | in tumor progression in carcinomas | |
| | and cutaneous melanoma (Johnson J P. | |
| | Cancer Metastasis Rev 1999; 18(3): | |
| | 345-57). | |
| sbg1563294bcadherin | An embodiment of the invention is | Cancer, infection, |
| | the use of sbg1563294bcadherin in | autoimmune |
| | epithelial polarization, epithelial | disorder, |
| | sheet or tube fusion, cell migration, | hematopoietic |
| | cell sorting, and axonal patterning. | disorder, wound |
| | A close homologue of | healing disorders and |
| | sbg1563294bcadherin is desmocollin, | inflammation |
| | a memeber of the cadherin super- | diseases |
| | family. Antisense expression of a | |
| | desmocollin gene in MDCK cells has | |
| | showed the alteration in desmosome | |
| | plaque assembly (Roberts G A, Burdett | |
| | I D, Pidsley S C, King I A, Magee A I, | |
| | Buxton R S. Eur J Cell Biol 1998 Jul.; | |
| | 76(3):192-203). Fat-like cadherins | |
| | were involved in epithelial morpho- | |
| | genesis, proliferation control, and | |
| | epithelial planar polarization (Tepass | |
| | U. Curr Opin Cell Biol 1999 Oct.; | |
| | 11(5):540-8, Takeshi Yagi and | |
| | Takeichi. Genes & Dev 2000. 14(10) | |
| | 1169-1180). Moreover, cadherin has | |
| | been implicated in tumor progression | |
| | in carcinomas and cutaneous melanoma | |
| | (Johnson J P. Cancer Metastasis Rev | |
| | 1999; 18(3):345-57). | |
| sbg1563509ECM | An embodiment of the invention is the | Cancer, infection, |
| | use of sbg1563509ECM to modulate the | autoimmune |
| | regulation of cell proliferation, | disorder, |
| | adhesion, migration, defective cell | hematopoietic |
| | migration, and chromosome instability. | disorder, wound |
| | Close homologues of sbg1563509ECM are | healing disorders, |
| | human fibulin-1, fibulin-2, chicken | tissue fragility, |
| | thrombospondin (TSP), mouse TSP-1, | abnormal escape |
| | and C. elegans hemicentin. The C- | reflex, |
| | terminal segment of human fibulin | mispositioning of |
| | contains eight consecutive calcium- | the vas deferens and |
| | binding EGF-like domains (Zhang R | uterus |
| | Z, Pan T C, Zhang Z Y, Mattei M G, | and inflammation |
| | Timpl R, Chu M L Genomics 1994 Jul. | diseases |
| | 15; 22(2):425-30). Fibulins | |
| | have been identified as basement | |
| | membrane and microfibrillar pro- | |
| | teins with a broad binding | |
| | repertoire for other extracellular | |
| | ligands (Giltay R, Timpl R, Kostka | |
| | G. Matrix Biol 1999 Oct.; 18(5): | |
| | 469-80). It was suggested that | |
| | the functional role of fibulin-1 | |
| | and fibulin-2 in bone marrow was | |
| | related to binding to the major | |
| | cell adhesion protein fibronectin | |
| | (Gu Y C, Nilsson K, Eng H, Ekblom | |
| | M. Br J Haematol 2000 May; 109(2): | |
| | 305-13). TSP contains VWFC | |
| | domains and EGF-like domains at | |
| | its N-terminal segment (Lawler J, | |
| | Duquette M, Ferro P. J Biol Chem | |
| | 1991 May 5; 266(13):8039-43). | |
| | TSP is a multifunction adhesive | |
| | protein with the ability to bind | |
| | proteoglycans, cell surface re- | |
| | ceptors, other proteins, and | |
| | calcium ions and is involved in the | |
| | regulation of cell proliferation, | |
| | adhesion and migration (Bornstein | |
| | P. J Cell Biol 1995 Aug.; 130 (3): | |
| | 503-6). TSP, as a endogenous | |
| | angiogenesis inhibitor, has been | |
| | shown to inhibit proliferation and | |
| | migration of vascular endothelial | |
| | cells in vitro and inhibit neovas- | |
| | cularization and tumor growth in | |
| | vivo (Streit, M., Riccardi L., | |
| | Velasco, P., Brown, L. F., | |
| | Hawighorst, T., Bornstein, P. and | |
| | Detmar, M. 1999. Proc. Natl Acad. | |
| | Sci. USA, 96, 14888-14893). TSP-1 | |
| | was found to suppresses wound | |
| | healing and granulation tissue | |
| | formation in the skin of transgenic | |
| | mice (Michael Streit, Paula | |
| | Velasco, Lucia Riccardi, Lisa | |
| | Spencer, Lawrence F. Brown, | |
| | Lauren Janes, Bernhard Lange- | |
| | Asschenfeldt, Kiichiro Yano, | |
| | Thomas Hawighorst, Luisa | |
| | Iruela-Arispe and Michael Detmar. | |
| | the EMBO Journal, Vol. 19, No. | |
| | 13 pp. 3272-3282, 2000). Mutants | |
| | of hemicentin, a conserved extra- | |
| | cellular member of the immuno- | |
| | globulin superfamily in | |
| | Caenorhabditis elegans, are shown | |
| | to cause a syndrome of tissue | |
| | fragility, defective cell | |
| | migration, chromosome insta- | |
| | bility, abnormal escape reflex, | |
| | mispositioning of the vas | |
| | deferens, uterus, mitotic chromo- | |
| | some loss, and multinucleate | |
| | cells in the germline (Vogel | |
| | B E, Hedgecock E M. Development | |
| | 2001 Mar.; 128 (6):883-894). | |
| sbg1552159Serprotease | An embodiment of the invention is | Cancer, infection, |
| | the use of sbg1552159Serprotease | autoimmune |
| | in modulating the various | disorder, |
| | enzyme activities as discussed | hematopoietic |
| | herein. Close homologues of | disorder, wound |
| | sbg1552159Serprotease are rat | healing disorders, |
| | serine protease, and Xenopus | and inflammation |
| | laevis ovochymase and oviductin | diseases |
| | which are both serine protease. | |
| | Serine proteases exist as pre- | |
| | cursors that are activated by | |
| | specific and limited proteo- | |
| | lysis, allowing regulation of | |
| | enzyme activity. Examples of | |
| | this type of regulation include | |
| | blood coagulation, fibrinolysis, | |
| | complement activation, and | |
| | trypsinogen activation by entero- | |
| | peptidase in digestion. The | |
| | precise control of these activa- | |
| | tion processes is crucial for | |
| | normal physiological enzymatic | |
| | function; misregulation of these | |
| | enzymes can lead to pathological | |
| | conditions (Takeuchi T, Shuman | |
| | M A, Craik C S. Proc Natl Acad | |
| | Sci USA 1999 Sep. 28; 96(20): | |
| | 11054-61). Evidence supports | |
| | a role for serine proteases, such | |
| | as blood-derived proteases throm- | |
| | bin, tissue plasminogen activator | |
| | and plasmin by entering into brain | |
| | tissue during cerebrovascular | |
| | insult, and causing the sequence | |
| | of events that can lead to glial | |
| | scarring, edema, seizure and | |
| | neuronal death (Gingrich M B, | |
| | Traynelis S F. Trends Neurosci | |
| | 2000 Sep.; 23(9):399-407). | |
| | Ovochymase, with chymotrypsin- | |
| | like (Phe-X) substrate specificity, | |
| | was translated as part of an | |
| | unusual polyprotein proenzyme | |
| | and released during egg activation. | |
| | Lindsay L L, Yang J C, Hedrick J L. | |
| | Proc Natl Acad Sci USA 1999 Sep. | |
| | 28; 96(20):11253-8). Oviductin, | |
| | the Xenopus laevis oviductal pro- | |
| | tease was observed to process egg | |
| | envelope glycoprotein gp43 to a | |
| | sperm-penetrable form, increases | |
| | sperm binding to envelopes | |
| | (Lindsay L L, Wieduwilt M J, | |
| | Hedrick J L. Biol Reprod 1999 | |
| | Apr.; 60(4):989-95). | |
| sbg1551159thymosinb4 | An embodiment of the invention is | Cancer, infection, |
| | the use of sbg1551159thymosinb4 | autoimmune |
| | in regulating rapid actin polymer- | disorder, |
| | ization/depolymerization processes | hematopoietic |
| | implicated in cell motility. A | disorder, wound |
| | close homologue of | healing disorders, |
| | sbg1551159thymosinb4 is thymosin | and inflammation |
| | b4. Thymosin b4 is a small actin- | diseases |
| | binding protein of 43 residues | |
| | whose role consists of sequester- | |
| | ing a large pool of unpolymerized | |
| | actin in cells (Fechheimer, M. & | |
| | Zigmond, S. H. J. Cell Biol. 1993. | |
| | 123, 1-5). In mammalian tissues, | |
| | it forms a 1:1 complex with ATP-G- | |
| | actin (Safer, D., Elzinga, M. & | |
| | Nachmias, V. T. J. Biol. Chem. | |
| | 1991. 266, 4029-4032). In | |
| | Xenopus laevis, thymosin beta 4 | |
| | mRNA was maternally present at a | |
| | low level and the transcript | |
| | became abundant after gastrula- | |
| | tion (Yamamoto M, Shoda A, | |
| | Minamino N, Matsuo H, Nishimatsu | |
| | S, Ueno N, Murakami K. Biochem | |
| | Biophys Res Commun 1992 Apr. 15; | |
| | 184(1):93-9). The oxidized | |
| | thymosin beta4 (thymosin beta 4 | |
| | sulfoxide) generated by monocytes | |
| | in the presence of glucocorti- | |
| | coids was demonstrated to contain | |
| | attenuated intracellular G-actin | |
| | sequestering activity but greatly | |
| | enhanced extracellular signal to | |
| | inhibit an inflammatory response | |
| | (Young J D, Lawrence A J, MacLean | |
| | A G, Leung B P, McInnes I B, Canas | |
| | B, Pappin D J, Stevenson R D. Nat | |
| | Med 1999 Dec.; 5(12):1424-7). | |
| | Further more, thymosin beta 4 | |
| | stimulated keratinocyte migration | |
| | in the Boyden chamber assay and | |
| | enhanced wound healing in a rat | |
| | full thickness wound model | |
| | (Malinda K M, Sidhu G S, Mani H, | |
| | Banaudha K, Maheshwari R K, | |
| | Goldstein A L, Kleinman H K. J | |
| | Invest Dermatol 1999 Sep.; | |
| | 113(3):364-8). | |
| SBh1686954.SOCS | An embodiment of the invention | Hyperproliferation, |
| | is the use of SBh1686954.SOCS | systemic |
| | in potential treatment for | autoimmune |
| | disease, injury, or abnormality | disease, |
| | involving cytokine mediated | hyperimmunity, |
| | cellular responsivenes, such as, | developmental |
| | hyperimmunity, immunosuppression, | abnormality |
| | allergies and hypertension. Close | |
| | homologues of SBh1686954.SOCS are | |
| | SOCS. SOCS can be used for | |
| | naturally occuring antibodies in | |
| | autoimmune diseases as well as | |
| | helpful for diagnosis of cancer | |
| | (W09820023-A1, submitted by Walter | |
| | and Eliza institute of Medical | |
| | Research). | |
| sbg1548844slit | An embodiment of the invention | Alzheimer's disease, |
| | is the use of sbg1548844slit to | cancer, |
| | regulate the formation and | gastrointestinal |
| | maintenance of the nervous and | ulceration, |
| | endocrine systems by protein- | neuropathy, wound |
| | protein interactions. Close | healing disorders and |
| | homologues of sbg1548844slit | inflammation |
| | are human Slit-1, -2, and -3, | diseases |
| | a leucine-rich repeat super- | |
| | family. In Drosophila embryo- | |
| | genesis, the slit gene had been | |
| | shown to play a critical role | |
| | in CNS midline formation. Human | |
| | Slit-1, -2, and -3 mRNAs were | |
| | exclusively expressed in the | |
| | brain, spinal cord, and thyroid, | |
| | respectively (Itoh A, Miyabayashi | |
| | T, Ohno M, Sakano S. Brain Res | |
| | Mol Brain Res 1998 Nov. 20; | |
| | 62(2):175-86). Similiar | |
| | leucine-rich protein GAC1 | |
| | was also found to be amplified | |
| | and overexpressed in malignant | |
| | gliomas (Almeida A, Zhu X X, Vogt | |
| | N, Tyagi R, Muleris M, Dutrillaux | |
| | A M, Dutrillaux B, Ross D, Malfoy | |
| | B, Hanash S. Oncogene 1998 Jun. | |
| | 11; 16(23):2997-3002). Slit | |
| | proteins were recently identified | |
| | as important regulators of axon | |
| | guidance and cell migration in | |
| | Drosophila and vertebrates, they | |
| | were simultaneously identified | |
| | as negative regulators, repell- | |
| | ing various axonal and cell | |
| | migrations in both invertebrates | |
| | and vertebrates, and as positive | |
| | regulators, stimulating branching | |
| | and extension of at least one | |
| | class of axons in vertebrates | |
| | (Brose K, Tessier-Lavigne M. | |
| | Curr Opin Neurobiol 2000 Feb.; | |
| | 10(1):95-102). | |
| sbg1545249GGT | An embodiment of the invention | Cancer, infection, |
| | is the use of sbg1545249GGTto | autoimmune |
| | regulate the gamma-glutamyl | disorder, |
| | cycle and the conjugation of | hematopoietic |
| | carcinogens and toxins. A close | disorder, wound |
| | homologue of sbg1545249GGT is | healing disorders, |
| | Gamma-glutamyltranspeptidase. | and inflammation |
| | Gamma-glutamyltranspeptidase | |
| | (GGT) catalyzes the first step | |
| | in the extracellular breakdown | |
| | of Glutathione (GSH) into | |
| | its constituent amino acids, | |
| | which can then be transported | |
| | into the cell and used to main- | |
| | tain the level of intracellular | |
| | GSH. GSH, the most abundant | |
| | intracellular nonprotein thiol, | |
| | is the major reductant of | |
| | cellular thiol groups and parti- | |
| | cipates in many important bio- | |
| | logical processes, including | |
| | the gamma-glutamyl cycle and | |
| | the conjugation of carcinogens | |
| | and toxins (Potdar P D, Andrews | |
| | K L, Nettesheim P, Ostrowski L | |
| | E. Am J Physiol 1997 Nov.; 273(5 | |
| | Pt 1):L1082-9). Mice deficient | |
| | in GGT appear normal at birth | |
| | but grow slowly and suffer pre- | |
| | mature death, demonstrating the | |
| | importance of GGT to normal | |
| | homeostasis (Lieberman, M. W., | |
| | A. L. Wiseman, Z. -Z. Shi, B. Z. | |
| | Carter, R. Barrios, C. -N. Ou, | |
| | P. Chevez-Barrios, Y. Wang, G. M. | |
| | Habib, J. C. Goodman, S. L. | |
| | Huang, R. M. Lebovitz, and M. M. | |
| | Matzuk. Proc. Natl. Acad. Sci. | |
| | USA 93:7923-7926, 1996). | |
| | Increased GGT activity is also | |
| | widely used as a marker of | |
| | preneoplastic lesions in the | |
| | liver during chemical carcino- | |
| | genesis (Hanigan, M. H., and H. | |
| | C. Pitot. Carcinogenesis 6: | |
| | 165-172, 1985). | |
| SBh1813899.Y82017 | An embodiment of the invention | Hyperproliferation, |
| | is the use of SBh1813899.Y82017 | Breast and Prostate |
| | to inhibit the development of | cancer |
| | prostate cancer. Close homologues | |
| | of SBh1813899.Y82017 contain | |
| | cytostatic activity and have | |
| | been identified as potential | |
| | vaccines and gene therapies | |
| | (WO200004149-A2, Human immuno- | |
| | genic prostate tumour protein | |
| | sequence SEQ ID NO: 378 | |
| | HARLOCKER S L, MITCHAM J L, | |
| | XU J, YUQIU J, DILLON D C: | |
| | CORIXA CORP,). | |
| sbg1536324mapkkk | An embodiment of the invention | Cancer, infection, |
| | is the use of sbg1536324mapkkk | autoimmune |
| | in mediating cell proliferation, | disorder, |
| | cell differentiation, and cell | hematopoietic |
| | survival by regulating signaling | disorder, wound |
| | pathways activated by receptor | healing disorders, |
| | protein tyrosine kinases. Close | and inflammation |
| | homologues of sbg1536324mapkkk | diseases |
| | are mitogen-activated protein | |
| | (MAP) kinases. Examples include: | |
| | Mitogen-activated protein kinase | |
| | kinase kinase 10, MLK2, a mixed | |
| | lineage kinase 2 (tyr and ser/thr | |
| | specificity) with a Src homology | |
| | 3 (SH3) domain at the N-terminus | |
| | followed by leucine zipper domains | |
| | and a proline rich domain (Dorow | |
| | D S, Devereux L, Tu G F, Price G, | |
| | Nicholl J K, Sutherland G R, | |
| | Simpson R J. Eur J Biochem 1995 | |
| | Dec. 1; 234(2):492-500). It has | |
| | been reported that MLK2 can | |
| | directly bind and mediate activa- | |
| | tion of MKK7 and SEK1, which in | |
| | turn induces JNK activation (Syu- | |
| | ichi Hirai, Kumi Noda, Tetsuo | |
| | Moriguchi, Eisuke Nishida, Akio | |
| | Yamashita, Tetsuya Deyama, Keiko | |
| | Fukuyama, and Shigeo Ohno. J Biol | |
| | Chem, Vol. 273, Issue 13, 7406- | |
| | 7412, Mar. 27, 1998). The JNK | |
| | pathway is required for the normal | |
| | regulation of AP-1 transcriptional | |
| | activity (Yang, D., Tournier, C., | |
| | Wysk, M., Lu, H. T., Xu, J., | |
| | Davis, R. J. & Flavell, R. A. | |
| | 1997. Proc. Natl. Acad. Sci. USA | |
| | 94, 3004-3009) and has been impli- | |
| | cated in the stress-induced apop- | |
| | tosis of neurons (Riesgo-Escovar, | |
| | J. R., Jenni, M., Fritz, A. & | |
| | Hafen, E. (1996) Genes Dev. 10, | |
| | 2759-2768), the malignant | |
| | transformation of pre-B cells | |
| | (Xia, Z., Dickens, M., Raingeaud, | |
| | J., Davis, R. J. & Greenberg, M. | |
| | E. (1995) Science 270, 1326- | |
| | 133137), and the expression of | |
| | E-selectin by endothelial cells | |
| | (Read, M. A., Whitely, M. Z., | |
| | Gupta, S., Pierce, J. W., Best, | |
| | J., Davis, R. J. & Collins, T. | |
| | (1997) J. Biol. Chem. 272, 2753- | |
| | 2761). | |
| gsk1810944BrCaAg | An embodiment of the invention | Breast and prostate |
| | is the use of gsk1810944BrCaAg | cancer |
| | to inhibit the development of | |
| | prostate cancer. Close homo- | |
| | logues of gsk1810944BrCaAg | |
| | contain cytostatic activity | |
| | and have been identified as | |
| | potential vaccines and gene | |
| | therapies (WO200004149-A2, | |
| | Human immunogenic prostate | |
| | tumour protein sequence SEQ | |
| | ID NO: 378 HARLOCKER S L, | |
| | MITCHAM J L, XU J, YUQIU J, | |
| | DILLON D C: CORIXA CORP,). | |
| sbg1535914AIP | An embodiment of the inven- | Cancer, neurological |
| | tion is the use of | diseases, |
| | sbg1535914AIP in preventing | autoimmune |
| | and/or treating neuronal | disorders, viral |
| | loss in animal models having | infections, and |
| | head injury, stroke, spinal | hematological |
| | cord trauma, and Alzheimer's | diseases |
| | disease. Close homologues of | |
| | sbg1535914AIP are mouse cas- | |
| | pase-3 and -7. The central | |
| | mechanisms of IAP apoptotic | |
| | suppression appeared to be | |
| | through direct caspase and | |
| | pro-caspase inhibition (pri- | |
| | marily caspase 3 and 7) and | |
| | modulation of and by the | |
| | transcription factor NF- | |
| | kappaB. It has been demon- | |
| | strated that X-linked | |
| | inhibitor of apoptosis pro- | |
| | tein (Xiap) was an important | |
| | element in the control of | |
| | ovarian tumor growth and | |
| | might be a point of regula- | |
| | tion for cisplatin in the | |
| | induction of apoptosis (Li | |
| | J, Feng Q, Kim J M, | |
| | Schneiderman D, Liston P, | |
| | Li M, Vanderhyden B, Faught | |
| | W, Fung M F, Senterman M, | |
| | Korneluk R G, Tsang B K. | |
| | Endocrinology 2001 Jan. 1; | |
| | 142(1):370-380). The strong- | |
| | est evidence for IAP involve- | |
| | ment in cancer was observed | |
| | in the IAP called survivin. | |
| | Although not observed in | |
| | adult differentiated tissue, | |
| | survivin was present in most | |
| | transformed cell lines and | |
| | cancers tested to date. Sur- | |
| | vivin was shown to inhibit | |
| | caspase directly and apop- | |
| | tosis in general. Moreover, | |
| | survivin protein levels corre- | |
| | lated inversely with 5 year | |
| | survival rates in colorectal | |
| | cancer. Recent data has also | |
| | implicated survivin in cell | |
| | cycle control. The IAPs have | |
| | been shown to be induced by | |
| | NF-kappaB or v-Rel in mul- | |
| | tiple cell lines and con- | |
| | versely, HIAP1 and HIAP2 have | |
| | been shown to activate NF- | |
| | kappaB possibly forming a | |
| | positive feed-back loop | |
| | (LaCasse E C, Baird S, | |
| | Korneluk R G, MacKenzie A E. | |
| | Oncogene 1998 Dec. 24; | |
| | 17(25):3247-59). Mice null | |
| | for caspase-3 displayed con- | |
| | siderable neuronal expansion | |
| | usually resulting in death | |
| | by the second week of life. | |
| | Consistent with the observa- | |
| | tion that apoptosis plays a | |
| | central role in human neuro- | |
| | degenerative disease, cas- | |
| | pase-3 activation has recent- | |
| | ly been observed in stroke, | |
| | spinal cord trauma, head in- | |
| | jury and Alzheimer's disease. | |
| | In severe spinal muscular | |
| | atrophy (SMA), the neuronal | |
| | specific inhibitor of apop- | |
| | tosis (IAP) family member | |
| | known as NAIF was often | |
| | dysfunctional due to missense | |
| | and truncation mutations | |
| | (Robertson G S, Crocker S J, | |
| | Nicholson D W, Schulz J B. | |
| | Brain Pathol 2000 Apr.; | |
| | 10(2):283-92). Taken to- | |
| | gether, these findings | |
| | suggest that anti-apop- | |
| | totic strategies may some | |
| | day have utility in the | |
| | treatment of cancer and | |
| | neurodegenerative diseases. | |
| sbg1530894aCE | An embodiment of the inven- | Cancer, infection, |
| | tion is the use of | metabolic diseases, |
| | sbg1530894aCE in modulating | and sperm |
| | cancerous cells and alter- | disfunction |
| | ing sperm differentiation | |
| | and maturation. Close | |
| | homologues of sbg1530894aCE | |
| | are mammalian carboxyles- | |
| | terases. Carboxylesterase | |
| | overexpression was observed | |
| | in the male reproductive | |
| | tract of different species. | |
| | Based on a well-known role | |
| | of carboxylesterases in de- | |
| | toxification of environmental | |
| | chemicals such as organophos- | |
| | phate pesticides, it was pro- | |
| | posed that various male | |
| | genital tract carboxyles- | |
| | terases was able to protect | |
| | the male reproductive system | |
| | against xenobiotic influences | |
| | that could provoke its dys- | |
| | function, thus altering sperm | |
| | differentiation and maturation | |
| | (Mikhailov A T, Torrado M. | |
| | Reprod Fertil Dev 1999; | |
| | 11(3):133-45). Mammalian | |
| | carboxylesterases represent a | |
| | multigene family, the products | |
| | of which are localized in the | |
| | endoplasmic reticulum of many | |
| | tissues. Multiple carboxyles- | |
| | terases are major determinants | |
| | of the pharmacokinetic behavior | |
| | of most therapeutic agents con- | |
| | taining ester or amide bonds. | |
| | Toxic metabolites converted | |
| | from systemically administered | |
| | prodrugs by recombinant adeno- | |
| | viruses expressing carboxyles- | |
| | terase (CE) in the local tumor | |
| | milieu has been shown to sup- | |
| | press tumor growth and attain | |
| | regional control in a single | |
| | organ (Crystal R G. Cancer | |
| | Chemother Pharmacol 1999; | |
| | 43 Suppl: S90-9). It was | |
| | proven to be a useful strategy | |
| | for achieving high local con- | |
| | centrations of the toxic | |
| | product while avoiding the | |
| | systemic toxicity that limits | |
| | the use of chemotherapy agents. | |
| | Thus, altering the activity | |
| | of CE class has important | |
| | clinical implications. | |
| sbg1530894bCE | An embodiment of the invention | Cancer, infection, |
| | is the us of sbg1530894bCE in | metabolic diseases, and sperm |
| | modulating cancerous cells and | disfunction |
| | altering sperm differentiation | |
| | and maturation. Close homo- | |
| | logues of sbg1530894bCE are | |
| | mammalian carboxylesterases. | |
| | Carboxylesterase overexpres- | |
| | sion was observed in the male | |
| | reproductive tract of differ- | |
| | ent species. Based on a well- | |
| | known role of carboxyles- | |
| | terases in detoxification of | |
| | environmental chemicals such | |
| | as organophosphate pesticides, | |
| | it was proposed that various | |
| | male genital tract carboxyles- | |
| | terases was able to protect | |
| | the male reproductive system | |
| | against xenobiotic influences | |
| | that could provoke its dys- | |
| | function, thus altering sperm | |
| | differentiation and maturation | |
| | (Mikhailov A T, Torrado M. | |
| | Reprod Fertil Dev 1999; 11(3): | |
| | 133-45). Mammalian carboxyl- | |
| | esterases represent a multigene | |
| | family, the products of which | |
| | are localized in the endoplas- | |
| | mic reticulum of many tissues. | |
| | Multiple carboxylesterases are | |
| | major determinants of the | |
| | pharmacokinetic behavior of | |
| | most therapeutic agents con- | |
| | taining ester or amide bonds. | |
| | Toxic metabolites converted | |
| | from systemically administered | |
| | prodrugs by recombinant adeno- | |
| | viruses expressing carboxyles- | |
| | terase (CE) in the local tumor | |
| | milieu has been shown to sup- | |
| | press tumor growth and attain | |
| | regional control in a single | |
| | organ (Crystal R G. Cancer | |
| | Chemother Pharmacol 1999; | |
| | 43 Suppl: S90-9). It was | |
| | proven to be a useful strategy | |
| | for achieving high local | |
| | concentrations of the toxic | |
| | product while avoiding the | |
| | systemic toxicity that limits | |
| | the use of chemotherapy | |
| | agents. Thus, altering the | |
| | activity of CE class has | |
| | important clinical impli- | |
| | cations. | |
| gsk.1813134.NaI | An embodiment of the inven- | Breast cancer, |
| | tion is the use of | thyroid disorders, |
| | gsk.1813134.NaI in the diag- | e.g. hypo- |
| | nosis and treatment of | thyroidism, hyper- |
| | thyroid disorders, e.g. | thyroidis, immune/ |
| | hypo-thyroidism and hyper- | inflammatory |
| | thyroidis. In addition, | disorders, |
| | gsk.1813134.NaI may be | hyperproliferation, |
| | useful in the prevention | and Prostate cancer |
| | and treatment of membrane | |
| | transport disorders and | |
| | immune/inflammatory dis- | |
| | orders. Close homologues | |
| | of gsk.1813134.NaI are | |
| | known to be specifically | |
| | expressed in breast cancer | |
| | cells. (W09728175-A1, Dai, | |
| | G., Levy, O., Carrasco, | |
| | N.; University of Yeshiva | |
| | Einstein College, | |
| | W0200026245, Azimzai, Y., | |
| | Corley, N. C., Guegler, K. | |
| | J., et. al. Incyte | |
| | Pharmaceutical Inc. | |
| gsk1811484BrCaAg | An embodiment of the inven- | Breast and prostate |
| | tion is the use of | cancer |
| | gsk1811484BrCaAg to inhibit | |
| | the development of prostate | |
| | cancer. Close homologues of | |
| | gsk1811484BrCaAg contain | |
| | cytostatic activity and | |
| | have been identified as | |
| | potential vaccines and gene | |
| | therapies (WO200004149-A2, | |
| | Human immunogenic prostate | |
| | tumour protein sequence SEQ | |
| | ID NO: 378 HARLOCKER S L, | |
| | MITCHAM J L, XU J, YUQIU J, | |
| | DILLON D C: CORIXA CORP,). | |
| sbg1529984aSproteinase | An embodiment of the inven- | Cancer, infection, |
| | tion is the use of | autoimmune |
| | sbg1529984aSproteinase, a | disorder, |
| | proteinase, in mediating | hematopoietic |
| | cellular infiltration, | disorder, wound |
| | cytokine activation, tissue | healing disorders, |
| | damage, remodeling, and re- | inflammation, brain |
| | pair. Close homologues of | damages, colitis |
| | sbg1529984aSproteinase are | diseases |
| | testisin and serine pro- | |
| | teases. Testisin, a human | |
| | serine proteinase, was found | |
| | abundantly expressed only in | |
| | the testis, and was lost in | |
| | testicular tumors. It was | |
| | also aberrantly expressed in | |
| | some tumor cell lines of | |
| | non-testis origin (Hooper J | |
| | D, Bowen N, Marshall H, | |
| | Cullen L M, Sood R, Daniels | |
| | R, Stuttgen M A, Normyle J | |
| | F, Higgs D R, Kastner D L, | |
| | Ogbourne S M, Pera M F, | |
| | Jazwinska E C, Antalis T M. | |
| | Biochim Biophys Acta 2000 | |
| | Jun. 21; 1492(1):63-71). | |
| | Serine proteases were also | |
| | suggested to be involved the | |
| | sequence of events that lead | |
| | to glial scarring, edema, | |
| | seizure and neuronal death | |
| | (Gingrich M B, Traynelis S F. | |
| | Trends Neurosci 2000 Sep.; | |
| | 23(9):399-407). A clear ele- | |
| | vation in the levels and | |
| | activation of serine pro- | |
| | tease was also found in | |
| | colitis (Tarlton J F, | |
| | Whiting C V, Tunmore D, | |
| | Bregenholt S, Reimann J, | |
| | Claesson M H, Bland P W. | |
| | Am J Pathol 2000 Dec.; | |
| | 157(6):1927-35). | |
| sbg1529984bSproteinase | An embodiment of the inven- | Cancer, infection, |
| | tion is the use of | autoimmune |
| | sbg1529984bSproteinase, a | disorder, |
| | proteinase, in mediating | hematopoietic |
| | cellular infiltration, | disorder, wound |
| | cytokine activation, tissue | healing disorders, |
| | damage, remodeling, and re- | inflammation, brain |
| | pair. Close homologues of | damages, colitis |
| | sbg1529984bSproteinase are | diseases |
| | testisin and serine pro- | |
| | teases. Testisin, a human | |
| | serine proteinase, was | |
| | found abundantly expressed | |
| | only in the testis, and was | |
| | lost in testicular tumors. | |
| | It was also aberrantly ex- | |
| | pressed in some tumor cell | |
| | lines of non-testis origin | |
| | (Hooper J D, Bowen N, | |
| | Marshall H, Cullen L M, | |
| | Sood R, Daniels R, Stuttgen | |
| | M A, Normyle J F, Higgs D R, | |
| | Kastner D L, Ogbourne S M, | |
| | Pera M F, Jazwinska E C, | |
| | Antalis T M. Biochim Biophys | |
| | Acta 2000 Jun. 21; 1492(1): | |
| | 63-71). Serine proteases | |
| | were also suggested to be | |
| | involved the sequence of | |
| | events that lead to glial | |
| | scarring, edema, seizure and | |
| | neuronal death (Gingrich M B, | |
| | Traynelis S F. Trends Neurosci | |
| | 2000 Sep.; 23(9):399-407). A | |
| | clear elevation in the levels | |
| | and activation of serine pro- | |
| | tease was also found in | |
| | colitis (Tarlton J F, | |
| | Whiting C V, Tunmore D, | |
| | Bregenholt S, Reimann J, | |
| | Claesson M H, Bland P W. | |
| | Am J Pathol 2000 Dec.; | |
| | 157(6):1927-35). | |
| SBGKIN113 | An embodiment of the inven- | Cone |
| | tion is the use of | phototransduction |
| | SBGKIN113 in regulating | deficiency |
| | vertebrate cone phototrans- | |
| | duction. Close homologues | |
| | of SBGKIN113 are mouse GRK1 | |
| | and squirrel GRK7. The G | |
| | protein-coupled receptor | |
| | kinases (GRKs) are critical | |
| | enzymes in the desensitiza- | |
| | tion of activated G protein- | |
| | coupled receptors. Mice | |
| | lacking GRK1 (rhodopsin kin- | |
| | ase) was found recently to | |
| | have profoundly slowed re- | |
| | covery of cone-driven re- | |
| | tinal responses suggesting | |
| | a major role of a specific | |
| | GRK in the inactivation of | |
| | vertebrate cone phototrans- | |
| | duction (Lyubarsky A L, Chen | |
| | C, Simon M I, Pugh E N. J | |
| | Neurosci 2000 Mar. 15; 20(6): | |
| | 2209-17). It was observed | |
| | that null mutation in GRK1 | |
| | slowed recovery kinetics of | |
| | rod and cone phototrans- | |
| | duction in man (Cideciyan A | |
| | V, Zhao X, Nielsen L, Khani | |
| | S C, Jacobson S G, Palczewski | |
| | K. Proc Natl Acad Sci USA | |
| | 1998 Jan. 6; 95(1):328-33). | |
| | Squirrel GRK7 was cloned | |
| | from retina and was shown to | |
| | phosphorylate bovine | |
| | rhodopsin in a light- | |
| | dependent manner similar to | |
| | bovine GRK1. The presence of | |
| | this kinase in cones suggests | |
| | that it could function in | |
| | cone cells as a cone opsin | |
| | kinase (Weiss E R, Raman D, | |
| | Shirakawa S, Ducceschi M H, | |
| | Bertram P T, Wong F, Kraft | |
| | T W, Osawa S. Mol Vis 1998 | |
| | Dec. 8; 4:27). | |
| gsk305961GDNa | An embodiment of the inven- | Cancer, infection, |
| | tion is the use of | autoimmune |
| | gsk305961GDNa, a member of | disorder, |
| | the serpin family, in | hematopoietic |
| | regulating neurite outgrowth. | disorder, wound |
| | gsk305961GDNa contains a | healing disorder, |
| | perfect representative of | inflammation, |
| | the serpin characteristic | neurological diseases |
| | pattern (pdoc00256). A close | (neuroinflammatory, |
| | homologue of gsk305961GDNa | neurodegenerative, |
| | is a glial derived nexin. | memory impairment, |
| | Serpins (SERine Proteinase | epilepsy, learning |
| | INhibitors) are specific | impairment, brain |
| | extracellular protease in- | trauma, stroke, |
| | hibitors with activity in | multiple sclerosis, |
| | the Cterminus. Glial derived | amyleotrophic lateral |
| | nexin (GDN, protease nexin I, | sclerosis, |
| | protease inhibitor 7), with | encephalitis, |
| | activity against thrombin, | Huntington's |
| | trypsin and urokinase, is | Disease, Down's |
| | known to promote neurite | Syndrome, |
| | outgrowth. (R W Scott, B L | Kallmann's disease), |
| | Bergman, A Bajpai, R T Hersh, | angiogenesis, cellular |
| | H Rodriguez, B N Jones, C | migration, ovulation |
| | Barreda, S Watts and J B | disorders |
| | Baker. Protease nexin. | |
| | Properties and a modified | |
| | purification procedure. J. | |
| | Biol. Chem., Vol. | |
| | 260, Issue 11, 7029-7034, | |
| | Jun., 1985; Monard, D., | |
| | NOday, E., Limat, A. & | |
| | Solomon, F. Prog. Brain | |
| | Res. 58, 359-364, 1983). | |
| gsk2402719_2395124BIG2 | An embodiment of the inven- | Cancer, |
| | tion is the use of | infection, autoimmune |
| | gsk2402719_2395124BIG2 a | disorder, |
| | member of the axon- | hematopoietic |
| | associated cell adhesion | disorder, wound |
| | molecule subfamily, in the | healing disorder, |
| | formation, maintenance and | inflammation, |
| | plasticity of functional | neurological diseases |
| | neuronal networks. A close | (neuroinflammatory, |
| | homologue of | neurodegenerative, |
| | gsk2402719_2395124BIG2 is a | memory impairment, |
| | rat subfamily member BIG2, | epilepsy, learning |
| | which is been shown to pro- | impairment, brain |
| | mote neurite outgrowth when | trauma, stroke, |
| | used as a substrate for | multiple sclerosis, |
| | neurons in vitro. (Yoshihara | amyleotrophic lateral |
| | Y, Kawasaki M, Tamada A, | sclerosis, |
| | Nagata S, Kagamiyama H, Mori | encephalitis, |
| | K. Overlapping and differen- | Huntington's |
| | tial expression of BIG-2, | Disease, Down's |
| | BIG-1, TAG-1, and F3: four | Syndrome, |
| | members of an axon-associ- | Kallmann's disease), |
| | ated cell adhesion molecule | angiogenesis, cellular |
| | subgroup of the immunoglobu- | migration, ovulation |
| | lin superfamily. J Neurobiol | disorders |
| | 1995 Sep.; 28(1):51-69). | |
| sbg18525LRRb | An embodiment of the inven- | Cancer, infection, |
| | tion is the use of | autoimmune |
| | sbg18525LRRb, a leucine- | disorder, |
| | rich repeat protein, in | hematopoietic |
| | regulating protein-protein | disorder, wound |
| | interactions, such as cell | healing disorder, |
| | adhesion or receptor-ligand | inflammation, |
| | binding. Close homologues | gastrointestinal |
| | of sbg18525LRRb are | ulceration, and |
| | connectin, slit, chaoptin, | diseases in spinal |
| | and toll. It is likely that | cord, thyroid gland, |
| | leucine-rich repeat (LRR) | heart, trachea, |
| | proteins such as connectin, | thymus, lymph |
| | slit, chaoptin, and toll | node, muscular |
| | have important roles in | system, and nervous |
| | neuronal development and | system |
| | the adult nervous system as | |
| | cell adhesion molecules | |
| | (Taguchi A, Wanaka A, Mori | |
| | T, Matsumoto K, Imai Y, | |
| | Tagaki T, Tohyama M, 1996, | |
| | Brain Res Mol Brain Res; | |
| | 35:31-4). At least one LRR | |
| | was shown to be specifi- | |
| | cally expressed on B cells, | |
| | suggesting its role in | |
| | immunization (Miyake K, | |
| | Yamashita Y, Ogata M, | |
| | Sudo T, Kimoto M, 1995. | |
| | J Immunol 154:3333-40). | |
| | Some studies have shown | |
| | that brain injury can | |
| | cause overexepression | |
| | of neuronal LRR, sug- | |
| | gesting that neuronal | |
| | LRR may be an important | |
| | component of the patho- | |
| | physiological response | |
| | to brain injury (Ishii | |
| | N, Wanaka A, Tohyama M, | |
| | 1996, Brain Res Mol | |
| | Brain Res 40:148-52). | |
| SBhACRP30c | An embodiment of the inven- | Cancer, obesity, |
| | tion is the use of | anorexia, |
| | SBhACRP30c in regulating | inflammation, |
| | inflammation, cell pro- | cardiovadcular |
| | liferation, cell death, | disease, growth |
| | immunity, and/or energy | abnormalities |
| | homeostatis. Close homo- | |
| | logues of SBhACRP30c are | |
| | ACRP30, chipmunk Hib27, | |
| | C1q complement proteins, | |
| | TNF, and other members | |
| | of the TNF superfamily. | |
| | ACRP30 (Adipocyte Comple- | |
| | ment-Related Protein of 30 | |
| | kDa) is made exclusively | |
| | in adipocytes, and its | |
| | expression is dysregulated | |
| | in various forms of obesity | |
| | (Hu, E, Liang, P and | |
| | Spiegelman, B M. J. Biol. | |
| | Chem 271, 10697-10703, | |
| | 1996). ACRP30 secretion is | |
| | acutely stimulated by in- | |
| | sulin (Scherer, P E, Williams | |
| | S., Fogliano, M., Baldini, | |
| | G. and Lodish, J Biol. Chem. | |
| | 270, 26746-26749, 1995) | |
| | and is repressed by chroni- | |
| | cally elevated levels of | |
| | insulin. A related molecule, | |
| | the Hib27 protein from Si- | |
| | berian chipmunks, seems also | |
| | to be involved in energy | |
| | homeostasis, as its expres- | |
| | sion is specifically ex- | |
| | tinguished during hiber- | |
| | nation (Takamatsu, N., | |
| | Ohba, K., Kondo, J., Kondo, | |
| | N., and Shiba, T. Mol. Cell | |
| | Biol. 13 1516-1521, 1993). | |
| | Recently, it has been shown | |
| | that the three dimensional | |
| | structure of ACRP30 is | |
| | superimposible with that of | |
| | the TNF's, suggesting that | |
| | these proteins may have a | |
| | similar function and mode | |
| | of action (Shapiro, L and | |
| | Scherer PE., Current Biology | |
| | 8, 335-338, 1997). TNF's | |
| | are known to play a role in | |
| | energy homeostasis, where | |
| | they are implicated in | |
| | cachexia, obesity and in | |
| | insulin resistance | |
| | (Hotamisligil G S., and | |
| | Spiegelman B M. Diabetes | |
| | (1994) 43, 1271-1278; | |
| | Teoman Uysal K., Wiesbrock | |
| | S M, Marina M W and | |
| | Hotamisligil G S., Nature | |
| | 389, 610-614, 1997). | |
| | Based on EST expression | |
| | data, SBHACRP30a is pri- | |
| | marily or exclusively | |
| | expressed in heart. | |
| sbg123493SLITc | An embodiment of the inven- | Diseases in spinal |
| | tion is the use of | cord, thyroid gland, |
| | sbg123493SLITc, a secreted | ovary, prostate, |
| | protein, in preventing and | renal gland, small |
| | treatment of diseases in | intestine, heart, |
| | spinal cord, thyroid gland, | trachea, thymus, |
| | ovary, prostate, renal | lymph node, |
| | gland, small intestine, | muscular system |
| | heart, trachea, thymus, | and colon, pineal |
| | lymph node, muscular system | tumors and |
| | and colon. Close homologues | alleviation of |
| | of sbg123493SLITc are rat | precocious puberty |
| | slit protein and pineal | |
| | gland specific gene-1 pro- | |
| | tein. This slit protein | |
| | binds Robo rec ptors and | |
| | has an evolutionarily con- | |
| | served role in repulsive | |
| | axon guidance. Pineal | |
| | gland specific gene-1 | |
| | protein, is used in the | |
| | treatment of pineal tumors | |
| | and alleviation of pre- | |
| | cocious puberty. (Brose, | |
| | K., Bland, K. S., Wang, K. | |
| | H., Arnott, D., Henzel, W. | |
| | Goodman, C. S., Tessier- | |
| | Lavigne, M. and Kidd, T. | |
| | Slit proteins bind Robo | |
| | receptors and have an | |
| | evolutionarily conserved | |
| | role in repulsive axon | |
| | guidance Cell 96 (6), | |
| | 795-806 (1999); | |
| | Application number | |
| | AAW09405, publication | |
| | date, 12 Dec. 1996, sub- | |
| | mitted by Human Genome | |
| | Science Inc) | |
| sbg102200MCTc | An embodiment of the inven- | Cancer, infection, |
| | tion is the use of | autoimmune |
| | sbg102200MCTc in regulating | disorder, |
| | pyruvate uptake. Close homo- | hematopoietic |
| | logues of sbg102200MCTc are | disorder, wound |
| | Chinese hamster and human | healing disorders, |
| | MCT1. Mouse H+-mono- | and inflammation |
| | carboxylate cotransporter | |
| | (MCT1) was cloned and se- | |
| | quenced from Ehrlich | |
| | Lettre tumour cells, the | |
| | sequence of MCT1 is 93% | |
| | and 87% identical to MCT1 | |
| | from Chinese hamster and | |
| | human, respectively. N- | |
| | glycanase-F treatment and | |
| | an in vitro translation | |
| | experiments demonstrated | |
| | that glycosylation was not | |
| | required for MCT1 function | |
| | (Carpenter L, Poole R C, | |
| | Halestrap A P. 1996. Biochim | |
| | Biophys Acta Mar. 13; | |
| | 1279(2):157-63). Chick | |
| | monocarboxylate transporter | |
| | MCT3 cloned from retinal | |
| | pigment epithelium (RPE) | |
| | cDNA library was found only | |
| | expressed in RPE cells. A | |
| | rat thyroid epithelial cell | |
| | line FRTL transfected with | |
| | pCl-neo/MCT3 showed enhanced | |
| | pyruvate uptake suggesting | |
| | that MCT3 may regulate lac- | |
| | tate levels in the inter- | |
| | photoreceptor space (Yoon H, | |
| | Fanelli A, Grollman E F, | |
| | Philp N J. 1997. Biochem | |
| | Biophys Res Commun May 8; | |
| | 234 (1):90-4). In human, | |
| | MCT2 has been implicated as | |
| | a primary pyruvate trans- | |
| | porter. The mRNAs of MCT1 | |
| | and MCT2 were found co- | |
| | expressed in various human | |
| | cancer cell lines, including | |
| | the hematopoietic lineages | |
| | HL60, K562, MOLT-4, | |
| | Burkitt's lymphoma Raji, and | |
| | solid tumor cells such as | |
| | SW480, A549, and G361. These | |
| | findings suggested that | |
| | human MCT1 and MCT2 may have | |
| | distinct biological roles | |
| | (Lin R Y, Vera J C, Chaganti | |
| | R S, Golde D W. 1998. J Biol | |
| | Chem Oct. 30; 273(44):28959- | |
| | 65). | |

Table IV. Quantitative, Tissue-Specific mRNA Expression Detected Using SybrMan

Quantitative, tissue-specific, mRNA expression patterns of the genes were measured using SYBR-Green Quantitative PCR (Applied Biosystems, Foster City, Calif.; see Schmittgen T. D. et al., Analytical Biochemistry 285:194-204, 2000) and human cDNAs prepared from various human tissues. Gene-specific PCR primers were designed using the first nucleic acid sequence listed in the Sequence List for each gene. The threshold cycle (Cₜ) is defined as the fractional cycle number at which the reporter fluorescence generated by cleavage of the probe reaches a threshold defined as 10 times the background. In cases sequence detection system software predicted more than one PCR product, Taqman was used for the specific PCR amplification as indicated under the specific genes.

In each gene's first subset table, two replicate measurements of gene of identification (GOI) mRNA were measured from various human tissues (column 3 and 4). The average GOI mRNA copies of the two replicates were made from each tissue RNA (column 5). The average amount of 18S rRNA from each tissue RNA was measured (column 6) and used for normalization. To make each tissue with the same amount of 50 ng of 18S rRNA, the normalization factor (column 7) was calculated by dividing 50 ng with the amount of 18S rRNA measured from each tissue (column 6). The mRNA copies per 50 ng of total RNA were obtained by multipling each GOI normalization factor and the average mRNA copies (column 8).

Fold changes shown in each gene's second subset table were only calculated for disease tissues which have a normal counterpart. There are blanks in the fold change column for all samples that do not have counterparts. In addition, the fold change calculations are the fold change in the disease sample as compared to the normal sample. Accordingly, there will not be a fold change calculation next to any of the normal samples. For patient matched cancer pairs (colon, lung, and breast), each tumor is compared to its specific normal counterpart. When patient-matched normal/disease pairs do not exist, each disease sample was compared back to the average of all the normal samples of that same tissue type. For example, normal brain from the same patient that provided Alzheimer's brain is not applicable. Three normal brain samples and 4 Alzheimer's brain samples are used in the fold change. Three normal samples were averaged, and each of the Alzheimer's samples was compared back to that average.

Abbreviations

ALZ Alzheimer's Disease

CT CLONTECH (1020 East Meadow Circle Palo Alto, Calif. 94303-4230, USA)

KC Sample prepared by GSK investigator

COPD chronic obstructive pulmonary disease

endo endothelial

VEGF vascular endothelial growth factor

bFGF basic fibroblast growth factor

BM bone marrow

osteo osteoblast

OA osteoarthritis

RA rheumatoid arthritis

PBL peripheral blood lymphocytes

PBMNC peripheral blood mononuclear cells

HIV human immunodeficiency virus

HSV Herpes simplex virus

HPV human papilloma virus

Gene Name sbg1571549cystatin-re

Moderate to low overall expression. Highest normal expression is seen in the whole brain, hypothalamus, liver, head of pancreas, small intestine, and uterus. This pattern of expression suggests that this gene may be involved in diabetes or other metabolic diseases. Highest disease expression is seen in the normal/tumor breast samples as well as in the RA and OA synovium samples. Upregulation in 2 of 4 breast tumor samples implicates this gene in breast cancer. Upregulation in 2 of 4 Alzheimer's brain samples suggests involvement in Alzheimer's disease. Downregulation in 3 of 3 COPD samples and in 2 of 4 asthmatic lung samples suggests a potential role for this gene in chronic obstructive pulmonary disorder and asthma. Upregulation in 3 of 3 disease heart samples implies an involvement in cardiovascular diseases such as non-obstructive and obstructive DCM and ischemia. Downregulation in the HIV-infected PBL cells and in the HSV-infected MRC5 cells suggests a role for this gene in HIV and HSV.

| | | | | | | | copies |
|---|---|---|---|---|---|---|---|
| | | Mean | Mean | | | | of mRNA |
| | | GOI | GOI | | | 50 ng/ | detected/ |
| Sample | Ct | copies | copies | Average | 18S | 18 S | 50 ng |
| sbg1571549- | (sample 1 | (sample | (sample | GOI | rRNA | rRNA | total |
| cystatin-re | and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 0.18 | 0.19 | 0.19 | 0.96 | 52.36 | 9.69 |
| Zenbio | | | | | | | |
| Adrenal Gland Clontech | 38.08, 38.33 | 0.8 | 0.7 | 0.75 | 0.61 | 81.97 | 61.48 |
| Whole Brain Clontech | 30.32, 30.44 | 59.6 | 55.83 | 57.72 | 7.24 | 6.91 | 398.58 |
| Fetal Brain Clontech | 39.19, 38.01 | 0.43 | 0.84 | 0.64 | 0.48 | 103.95 | 66.01 |
| Cerebellum Clontech | 39.49, 38.62 | 0.37 | 0.59 | 0.48 | 2.17 | 23.04 | 11.06 |
| Cervix | 38.35, 40 | 0.69 | 0.22 | 0.46 | 2.42 | 20.66 | 9.40 |
| Colon | 37.24, 33.17 | 1.28 | 12.27 | 6.78 | 2.71 | 18.45 | 125.00 |
| Endometrium | 38.93, 37.89 | 0.5 | 0.89 | 0.70 | 0.73 | 68.21 | 47.41 |
| Esophagus | 38.16, 36.9 | 0.77 | 1.55 | 1.16 | 1.37 | 36.50 | 42.34 |
| Heart Clontech | 38.51, 36.41 | 0.63 | 2.03 | 1.33 | 1.32 | 37.88 | 50.38 |
| Hypothalamus | 36.58, 37.03 | 1.85 | 1.44 | 1.65 | 0.32 | 155.28 | 255.43 |
| Ileum | 38.36, 33.12 | 0.69 | 12.61 | 6.65 | 2.58 | 19.38 | 128.88 |
| Jejunum | 32.5, 39.56 | 17.8 | 0.35 | 9.08 | 6.60 | 7.58 | 68.75 |
| Kidney | 35.43, 36.28 | 3.49 | 2.18 | 2.84 | 2.12 | 23.58 | 66.86 |
| Liver | 38.57, 32.25 | 0.61 | 20.46 | 10.54 | 1.50 | 33.33 | 351.17 |
| Fetal Liver Clontech | 38.06, 37.94 | 0.81 | 0.87 | 0.84 | 10.40 | 4.81 | 4.04 |
| Lung | 37.01, 34.41 | 1.45 | 6.15 | 3.80 | 2.57 | 19.46 | 73.93 |
| Mammary Gland | 33.49, 34 | 10.24 | 7.74 | 8.99 | 13.00 | 3.85 | 34.58 |
| Clontech | | | | | | | |
| Myometrium | 36.23, 38.44 | 2.24 | 0.66 | 1.45 | 2.34 | 21.37 | 30.98 |
| Omentum | 37.67, 37.81 | 1.01 | 0.93 | 0.97 | 3.94 | 12.69 | 12.31 |
| Ovary | 40, 34.28 | 0 | 6.63 | 3.32 | 4.34 | 11.52 | 38.19 |
| Pancreas | 36.22, 40 | 2.26 | 0 | 1.13 | 0.81 | 61.80 | 69.84 |
| Head of Pancreas | 32.37, 36.38 | 19.09 | 2.06 | 10.58 | 1.57 | 31.85 | 336.78 |
| Parotid Gland | 33.64, 39.31 | 9.46 | 0.41 | 4.94 | 5.48 | 9.12 | 45.03 |
| Placenta Clontech | 36.31, 33.17 | 2.14 | 12.29 | 7.22 | 5.26 | 9.51 | 68.58 |
| Prostate | 38.64, 38.17 | 0.59 | 0.76 | 0.68 | 3.00 | 16.67 | 11.25 |
| Rectum | 38.55, 38.15 | 0.62 | 0.77 | 0.70 | 1.23 | 40.65 | 28.25 |
| Salivary Gland Clontech | 40, 40 | 0.23 | 0 | 0.12 | 7.31 | 6.84 | 0.79 |
| Skeletal Muscle | 36.02, 39.17 | 2.52 | 0.44 | 1.48 | 1.26 | 39.68 | 58.73 |
| Clontech | | | | | | | |
| Skin | 35.55, 35.44 | 3.28 | 3.49 | 3.39 | 1.21 | 41.32 | 139.88 |
| Small Intestine Clontech | 37.51, 33.94 | 1.1 | 8.01 | 4.56 | 0.98 | 51.07 | 232.64 |
| Spleen | 36.06, 38.75 | 2.47 | 0.55 | 1.51 | 4.92 | 10.16 | 15.35 |
| Stomach | 34.58, 33.81 | 5.61 | 8.59 | 7.10 | 2.73 | 18.32 | 130.04 |
| Testis Clontech | 37.39, 37.34 | 1.18 | 1.21 | 1.20 | 0.57 | 87.87 | 105.01 |
| Thymus Clontech | 33.94, 37.28 | 8.01 | 1.25 | 4.63 | 9.89 | 5.06 | 23.41 |
| Thyroid | 35.69, 37.08 | 3.02 | 1.4 | 2.21 | 2.77 | 18.05 | 39.89 |
| Trachea Clontech | 37.53, 38.01 | 1.09 | 0.83 | 0.96 | 9.71 | 5.15 | 4.94 |
| Urinary Bladder | 38.1, 37.26 | 0.79 | 1.26 | 1.03 | 5.47 | 9.14 | 9.37 |
| Uterus | 31.16, 34.07 | 37.51 | 7.42 | 22.47 | 5.34 | 9.36 | 210.35 |
| genomic | 24.9 | 1208.23 | | | | | |
| b-actin | 26.07 | 630.69 | | | | | |
| 1.00E+05 | 17.45 | 100000 | | | | | |
| 1.00E+05 | 17.57 | 100000 | | | | | |
| 1.00E+04 | 21.26 | 10000 | | | | | |
| 1.00E+04 | 20.74 | 10000 | | | | | |
| 1.00E+03 | 24.49 | 1000 | | | | | |
| 1.00E+03 | 24.38 | 1000 | | | | | |
| 1.00E+02 | 29.46 | 100 | | | | | |
| 1.00E+02 | 28.37 | 100 | | | | | |
| 1.00E+01 | 36.37 | 10 | | | | | |
| 1.00E+01 | 32.51 | 10 | | | | | |
| 1.00E+00 | 37.91 | 1 | | | | | |
| 1.00E+00 | 37.27 | 1 | | | | | |
| NTC | 35.86 | −1 | | | | | |
| NTC | 39.15 | −1 | | | | | |

| | | | | copies of | | |
|---|---|---|---|---|---|---|
| | | | | mRNA | | Fold |
| | Reg | | | detected / | | Change |
| Sample | number | | Mean | 50 ng | | in |
| sbg1571549- | (GSK | | GOI | total | | Disease |
| cystatin-re | identifier) | Ct | copies | RNA | Sample | Population |
| colon normal GW98-167 | 21941 | 32.53 | 18.72 | 37.44 | colon normal | |
| colon tumor GW98-166 | 21940 | 32.71 | 16.83 | 33.66 | colon tumor | −1.11 |
| colon normal GW98-178 | 22080 | 35.41 | 3.3 | 6.60 | colon normal | |
| colon tumor GW98-177 | 22060 | 33.45 | 10.79 | 21.58 | colon tumor | 3.27 |
| colon normal GW98-561 | 23514 | 32.21 | 22.71 | 45.42 | colon normal | |
| colon tumor GW98-560 | 23513 | 35.14 | 3.89 | 7.78 | colon tumor | −5.84 |
| colon normal GW98-894 | 24691 | 31.7 | 30.93 | 61.86 | colon normal | |
| colon tumor GW98-893 | 24690 | 32.11 | 24.11 | 48.22 | colon tumor | −1.28 |
| lung normal GW98-3 | 20742 | 30.69 | 56.9 | 113.80 | lung normal | |
| lung tumor GW98-2 | 20741 | 34.24 | 6.67 | 13.34 | lung tumor | −8.53 |
| lung normal GW97-179 | 20677 | 33.59 | 9.89 | 19.78 | lung normal | |
| lung tumor GW97-178 | 20676 | 31.55 | 33.83 | 67.66 | lung tumor | 3.42 |
| lung normal GW98-165 | 21922 | 31.6 | 32.73 | 65.46 | lung normal | |
| lung tumor GW98-164 | 21921 | 32.03 | 25.34 | 50.68 | lung tumor | −1.29 |
| lung normal GW98-282 | 22584 | 35.05 | 4.11 | 8.22 | lung normal | |
| lung tumor GW98-281 | 22583 | 32.79 | 16.06 | 32.12 | lung tumor | 3.91 |
| breast normal GW00-392 | 28750 | 27.24 | 452.07 | 452.07 | breast normal | |
| breast tumor GW00-391 | 28746 | 28.37 | 229.53 | 459.06 | breast tumor | 1.02 |
| breast normal GW00-413 | 28798 | 26.81 | 585.37 | 585.37 | breast normal | |
| breast tumor GW00-412 | 28797 | 25.15 | 1596.82 | 3193.64 | breast tumor | 5.46 |
| breast normal GW00- | 27592-95 | 35.56 | 3.02 | 3.02 | breast normal | |
| 235:238 | | | | | | |
| breast tumor GW00- | 27588-91 | 29.85 | 93.86 | 93.86 | breast tumor | 31.08 |
| 231:234 | | | | | | |
| breast normal GW98-621 | 23656 | 32.1 | 24.26 | 48.52 | breast normal | |
| breast tumor GW98-620 | 23655 | 35.55 | 3.04 | 6.08 | breast tumor | −7.98 |
| brain normal BB99-542 | 25507 | 35.27 | 3.61 | 7.22 | brain normal | |
| brain normal BB99-406 | 25509 | 33.99 | 7.79 | 15.58 | brain normal | |
| brain normal BB99-904 | 25546 | 34.34 | 6.31 | 12.62 | brain normal | |
| brain stage 5 ALZ BB99- | 25502 | 33.03 | 13.84 | 27.68 | brain stage 5 ALZ | 2.34 |
| 874 | | | | | | |
| brain stage 5 ALZ BB99- | 25503 | 31.1 | 44.41 | 88.82 | brain stage 5 ALZ | 7.52 |
| 887 | | | | | | |
| brain stage 5 ALZ BB99- | 25504 | 32.56 | 18.44 | 36.88 | brain stage 5 ALZ | 3.12 |
| 862 | | | | | | |
| brain stage 5 ALZ BB99- | 25542 | 33.24 | 12.2 | 24.40 | brain stage 5 ALZ | 2.07 |
| 927 | | | | | | |
| CT lung | normal | 32.12 | 24.04 | 48.08 | CT lung Nml | |
| lung 26 | normal | 26.05 | 928.5 | | lung 26 Nml | |
| lung 27 | normal | 40 | 0 | 0.00 | lung 27 Nml | |
| lung 24 | COPD | 38.83 | 0.42 | 0.42 | lung 24 COPD | −38.16 |
| lung 28 | COPD | 40 | 0 | 0.00 | lung 28 COPD | −16.03 |
| lung 23 | COPD | 40 | 0 | 0.00 | lung 23 COPD | −16.03 |
| lung 25 | normal | 40 | 0 | 0.00 | lung 25 Nml | |
| asthmatic lung ODO3112 | 29321 | 40 | 0 | 0.00 | asthmatic lung | −16.03 |
| asthmatic lung ODO3433 | 29323 | 40 | 0.2 | 0.40 | asthmatic lung | −40.07 |
| asthmatic lung ODO3397 | 29322 | 33.96 | 7.93 | 15.86 | asthmatic lung | −1.01 |
| asthmatic lung ODO4928 | 29325 | 35.14 | 3.9 | 7.80 | asthmatic lung | −2.05 |
| endo cells | control | 34.25 | 6.65 | 6.65 | endo cells | |
| endo VEGF | | 36.2 | 2.05 | 2.05 | endo VEGF | −3.24 |
| endo bFGF | | 36.19 | 2.06 | 2.06 | endo bFGF | −3.23 |
| heart Clontech | normal | 38.63 | 0.48 | 0.96 | heart | |
| heart (T-1) ischemic | 29417 | 36.32 | 1.91 | 3.82 | heart (T-1) ischemic | 3.98 |
| heart (T-14) non- | 29422 | 34.07 | 7.41 | 14.82 | heart (T-14) non- | 15.44 |
| obstructive DCM | | | | | obstructive DCM | |
| heart (T-3399) DCM | 29426 | 34.43 | 5.97 | 11.94 | heart (T-3399) DCM | 12.44 |
| adenoid GW99-269 | 26162 | 34.29 | 6.47 | 12.94 | adenoid | |
| tonsil GW98-280 | 22582 | 37.3 | 1.06 | 2.12 | tonsil | |
| T cells PC00314 | 28453 | 36.43 | 1.79 | 3.58 | T cells | |
| PBMNC | | 40 | 0 | 0.00 | PBMNC | |
| monocyte | | 35.28 | 3.58 | 7.16 | monocyte | |
| B cells PC00665 | 28455 | 38.29 | 0.58 | 1.16 | B cells | |
| dendritic cells | 28441 | 34.09 | 7.31 | 14.62 | dendritic cells | |
| neutrophils | 28440 | 35.65 | 2.86 | 2.86 | neutrophils | |
| eosinophils | 28446 | 37.31 | 1.05 | 2.10 | eosinophils | |
| BM unstim | | 38.46 | 0.53 | 0.53 | BM unstim | |
| BM stim | treated | 40 | 0.2 | 0.20 | BM stim | −2.65 |
| osteo dif | treated | 37.81 | 0.78 | 0.78 | osteo dif | 0.78 |
| osteo undif | | 40 | 0 | 0.00 | osteo undif | |
| chondrocytes | | 32.19 | 22.93 | 57.33 | chondrocytes | |
| OA Synovium IP 12/01 | 29462 | 36.01 | 2.3 | 2.30 | OA Synovium | |
| OA Synovium NP10/01 | 29461 | 32.01 | 25.56 | 51.12 | OA Synovium | |
| OA Synovium NP57/00 | 28464 | 30.61 | 59.45 | 118.90 | OA Synovium | |
| RA Synovium | 28466 | 28.04 | 279.01 | 558.02 | RA Synovium | |
| NP03/01 | | | | | | |
| RA Synovium | 28467 | 30.26 | 73.55 | 147.10 | RA Synovium | |
| NP71/00 | | | | | | |
| RA Synovium | 28475 | 30.62 | 59.33 | 118.66 | RA Synovium | |
| NP45/00 | | | | | | |
| OA bone (biobank) | 29217 | 40 | 0 | 0.00 | OA bone (biobank) | |
| OA bone Sample 1 | J. Emory | 34.61 | 5.36 | 10.72 | OA bone | |
| OA bone Sample 2 | J. Emory | 35.81 | 2.6 | 5.20 | OA bone | |
| Cartilage (pool) | Normal | 31.89 | 27.57 | 55.14 | Nml Cartilage (pool) | |
| Cartilage (pool) | OA | 32.68 | 17.14 | 34.28 | OA Cartilage (pool) | −1.61 |
| PEL unifected | 28441 | 35.33 | 3.47 | 6.94 | PBL unifected | |
| PBL HTV IIIB | 28442 | 39.75 | 0.24 | 0.48 | PBL HIV IIIB | −14.46 |
| MRC5 uninfected | 29158 | 29.55 | 112.51 | 225.02 | MRC5 uninfected | |
| (100%) | | | | | (100%) | |
| MRC5 HSV strain F | 29178 | 33.51 | 10.36 | 20.72 | MRC5 HSV strain F | −10.86 |
| W12 cells | 29179 | 32.16 | 23.43 | 46.86 | W12 cells | |
| Keratinocytes | 29180 | 32.05 | 24.96 | 49.92 | Keratinocytes | |
| B-actin control | | 26.37 | 765.93 | | | |
| genomic | | 25.7 | 1146.09 | | | |
| 1.00E+05 | | 18.02 | 100000 | | | |
| 1.00E+05 | | 18.16 | 100000 | | | |
| 1.00E+04 | | 21.71 | 10000 | | | |
| 1.00E+04 | | 21.87 | 10000 | | | |
| 1.00E+03 | | 25.65 | 1000 | | | |
| 1.00E+03 | | 25.51 | 1000 | | | |
| 1.00E+02 | | 30.97 | 100 | | | |
| 1.00E+02 | | 30.1 | 100 | | | |
| 1.00E+01 | | 32.32 | 10 | | | |
| 1.00E+01 | | 38.11 | 10 | | | |
| 1.00E+00 | | 40 | 0 | | | |
| 1.00E+00 | | 34.22 | 1 | | | |
| NTC | | 38.19 | −1 | | | |

*lung 26 Normal has been omitted due to multiple amplification failures from that sample

| | Fold Change in Disease |
|---|---|
| | Population Relative to |
| Disease tissues | Normal |
| colon tumor | −1.11 |
| colon tumor | 3.27 |
| colon tumor | −5.84 |
| colon tumor | −1.28 |
| lung tumor | −8.53 |
| lung tumor | 3.42 |
| lung tumor | −1.29 |
| lung tumor | 3.91 |
| breast tumor | 1.02 |
| breast tumor | 5.46 |
| breast tumor | 31.08 |
| breast tumor | −7.98 |
| brain stage 5 ALZ | 2.34 |
| brain stage 5 ALZ | 7.52 |
| brain stage 5 ALZ | 3.12 |
| brain stage 5 ALZ | 2.07 |
| lung 24 COPD | −38.16 |
| lung 28 COPD | −16.03 |
| lung 23 COPD | −16.03 |
| asthmatic lung | −16.03 |
| asthmatic lung | −40.07 |
| asthmatic lung | −1.01 |
| asthmatic lung | −2.05 |
| endo VEGF | −3.24 |
| endo bFGF | −3.23 |
| heart (T-1) ischemic | 3.98 |
| heart (T-14) non-obstructive DCM | 15.44 |
| heart (T-3399) DCM | 12.44 |
| BM stim | −2.65 |
| osteo dif | 0.78 |
| OA Cartilage (pool) | −1.61 |
| PBL HTV IIIB | −14.46 |
| MRC5 HSV strain F | −10.86 |

Gene Name sbg1571549cystatin-re

Gene Name sbg1558434SRCR

Moderate to low overall expression. This gene is expressed fairly ubiquitously in all normal samples analyzed with the highest levels of expression seen in the whole brain, fetal brain, endometrium, and parotid gland. Highest disease expression is seen in the normal and Alzheimer's brain samples and in the RA and OA bone samples. Upregulation in 2 of 4 breast tumor samples implicates this gene in breast cancer. Downregulation in 1 of 3 COPD samples and in 1 of 4 asthmatic lung samples suggests a potential role for this gene in chronic obstructive pulmonary disorder and asthma. Downregulation in the HIV-infected PBL cells and in the HSV-infected MRC5 cells suggests a role for this gene in HIV and HSV.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample 1 | (sample | (sample | GOI | rRNA | 18 S | total |
| sbg1558434SRCR | and 2) | 1) | 2) | Copies | (ng) | rRNA(ng) | RNA |
| Subcutaneous | 38.36, 38.46 | 5.73 | 5.4 | 5.57 | 3.06 | 16.34 | 90.93 |
| Adipocytes Zenbio | | | | | | | |
| Subcutaneous Adipose | 39.06, 39.58 | 3.66 | 2.63 | 3.15 | 0.96 | 52.36 | 164.66 |
| Zenbio | | | | | | | |
| Adrenal Gland | 39.97, 38.01 | 1.44 | 7.19 | 4.32 | 0.61 | 81.97 | 353.69 |
| Clontech | | | | | | | |
| Whole Brain Clontech | 29.24, 29.36 | 2025.47 | 1877.04 | 1951.26 | 7.24 | 6.91 | 13475.52 |
| Fetal Brain Clontech | 37.04, 38.12 | 13.44 | 6.69 | 10.07 | 0.48 | 103.95 | 1046.26 |
| Cerebellum Clontech | 35.93, 36.29 | 27.41 | 21.74 | 24.58 | 2.17 | 23.04 | 566.24 |
| Cervix | 38.03, 40 | 7.11 | 1.83 | 4.47 | 2.42 | 20.66 | 92.36 |
| Colon | 37.31, 38.11 | 11.27 | 6.73 | 9.00 | 2.71 | 18.45 | 166.05 |
| Endometrium | 36.72, 38.11 | 16.48 | 6.76 | 11.62 | 0.73 | 68.21 | 792.63 |
| Esophagus | 40, 38.98 | 1.7 | 3.87 | 2.79 | 1.37 | 36.50 | 101.64 |
| Heart Clontech | 40, 40 | 0 | 0 | 0.00 | 1.32 | 37.88 | 0.00 |
| Hypothalamus | 40, 39.1 | 0 | 3.56 | 1.78 | 0.32 | 155.28 | 276.40 |
| Ileum | 36.38, 34.9 | 20.49 | 53.26 | 36.88 | 2.58 | 19.38 | 714.63 |
| Jejunum | 35.3, 34.3 | 41.09 | 78.41 | 59.75 | 6.60 | 7.58 | 452.65 |
| Kidney | 37.19, 40 | 12.16 | 0 | 6.08 | 2.12 | 23.58 | 143.40 |
| Liver | 38.75, 36.46 | 4.47 | 19.51 | 11.99 | 1.50 | 33.33 | 399.67 |
| Fetal Liver Clontech | 33.75, 34.3 | 111.17 | 78.04 | 94.61 | 10.40 | 4.81 | 454.83 |
| Lung | 36.52, 36.62 | 18.74 | 17.61 | 18.18 | 2.57 | 19.46 | 353.60 |
| Mammary Gland | 33.65, 32.72 | 118.53 | 215.49 | 167.01 | 13.00 | 3.85 | 642.35 |
| Clontech | | | | | | | |
| Myometrium | 36.02, 35.39 | 25.8 | 38.86 | 32.33 | 2.34 | 21.37 | 690.81 |
| Omentum | 36.03, 36.27 | 25.7 | 22.05 | 23.88 | 3.94 | 12.69 | 302.98 |
| Ovary | 35.07, 35.58 | 47.55 | 34.44 | 41.00 | 4.34 | 11.52 | 472.29 |
| Pancreas | 39.37, 40 | 3.01 | 0 | 1.51 | 0.81 | 61.80 | 93.02 |
| Head of Pancreas | 36.64, 36.61 | 17.38 | 17.7 | 17.54 | 1.57 | 31.85 | 558.60 |
| Parotid Gland | 32.39, 31.96 | 267.75 | 353.03 | 310.39 | 5.48 | 9.12 | 2832.03 |
| Placenta Clontech | 36.81, 36.33 | 15.55 | 21.26 | 18.41 | 5.26 | 9.51 | 174.95 |
| Prostate | 37.41, 40 | 10.56 | 0 | 5.28 | 3.00 | 16.67 | 88.00 |
| Rectum | 36.41, 37.24 | 20.1 | 11.83 | 15.97 | 1.23 | 40.65 | 648.98 |
| Salivary Gland | 35.89, 37.94 | 28.06 | 7.54 | 17.80 | 7.31 | 6.84 | 121.75 |
| Clontech | | | | | | | |
| Skeletal Muscle | 38.79, 40 | 4.37 | 0 | 2.19 | 1.26 | 39.68 | 86.71 |
| Clontech | | | | | | | |
| Skin | 37.85, 40 | 7.95 | 0 | 3.98 | 1.21 | 41.32 | 164.26 |
| Small Intestine | 40, 39.26 | 0 | 3.23 | 1.62 | 0.98 | 51.07 | 82.48 |
| Clontech | | | | | | | |
| Spleen | 36.13, 37.51 | 24.07 | 9.94 | 17.01 | 4.92 | 10.16 | 172.82 |
| Stomach | 37.71, 37.79 | 8.75 | 8.27 | 8.51 | 2.73 | 18.32 | 155.86 |
| Testis Clontech | 38.78, 37.36 | 4.39 | 10.94 | 7.67 | 0.57 | 87.87 | 673.55 |
| Thymus Clontech | 35.3, 35.43 | 41.14 | 37.7 | 39.42 | 9.89 | 5.06 | 199.29 |
| Thyroid | 38.37, 37.54 | 5.7 | 9.75 | 7.73 | 2.77 | 18.05 | 139.44 |
| Trachea Clontech | 37.12, 36.12 | 12.77 | 24.18 | 18.48 | 9.71 | 5.15 | 95.13 |
| Urinary Bladder | 36.06, 36.07 | 25.22 | 25.12 | 25.17 | 5.47 | 9.14 | 230.07 |
| Uterus | 36.75, 36.59 | 16.22 | 17.9 | 17.06 | 5.34 | 9.36 | 159.74 |
| genomic | 30.97 | 665.14 | | | | | |
| b-actin | 26.24 | 13919.08 | | | | | |
| 1.00E+05 | 21.78 | 100000 | | | | | |
| 1.00E+05 | 21.42 | 100000 | | | | | |
| 1.00E+04 | 26.73 | 10000 | | | | | |
| 1.00E+04 | 26.98 | 10000 | | | | | |
| 1.00E+03 | 30.72 | 1000 | | | | | |
| 1.00E+03 | 31.14 | 1000 | | | | | |
| 1.00E+02 | 35.32 | 100 | | | | | |
| 1.00E+02 | 37.01 | 100 | | | | | |
| 1.00E+01 | 38.96 | 10 | | | | | |
| 1.00E+01 | 38.41 | 10 | | | | | |
| 1.00E+00 | 39.65 | 1 | | | | | |
| 1.00E+00 | 37.39 | 1 | | | | | |
| NTC | 40 | −1 | | | | | |
| NTC | 37.08 | −1 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1558434SRCR | identifier) | Ct | copies | RNA | Sample | Population |
| colon normal GW98-167 | 21941 | 30.6 | 1522.06 | 3044.12 | colon normal | |
| colon tumor GW98-166 | 21940 | 28.65 | 4895.5 | 9791.00 | colon tumor | 3.22 |
| colon normal GW98-178 | 22080 | 32.98 | 366.41 | 732.82 | colon normal | |
| colon tumor GW98-177 | 22060 | 33.13 | 335.61 | 671.22 | colon tumor | −1.09 |
| colon normal GW98-561 | 23514 | 32.96 | 370.41 | 740.82 | colon normal | |
| colon tumor GW98-560 | 23513 | 32.32 | 545.47 | 1090.94 | colon tumor | 1.47 |
| colon normal GW98-894 | 24691 | 32.23 | 574.3 | 1148.60 | colon normal | |
| colon tumor GW98-893 | 24690 | 32.82 | 403.39 | 806.78 | colon tumor | −1.42 |
| lung normal GW98-3 | 20742 | 30 | 2180.92 | 4361.84 | lung normal | |
| lung tumor GW98-2 | 20741 | 29.78 | 2492.04 | 4984.08 | lung tumor | 1.14 |
| lung normal GW97-179 | 20677 | 29.53 | 2888.4 | 5776.80 | lung normal | |
| lung tumor GW97-178 | 20676 | 30.84 | 1317.26 | 2634.52 | lung tumor | −2.19 |
| lung normal GW98-165 | 21922 | 30.08 | 2083.87 | 4167.74 | lung normal | |
| lung tumor GW98-164 | 21921 | 28.84 | 4367.14 | 8734.28 | lung tumor | 2.10 |
| lung normal GW98-282 | 22584 | 30.99 | 1208.68 | 2417.36 | lung normal | |
| lung tumor GW98-281 | 22583 | 31.14 | 1104.34 | 2208.68 | lung tumor | −1.09 |
| breast normal GW00-392 | 28750 | 31.07 | 1150.42 | 1150.42 | breast normal | |
| breast tumor GW00-391 | 28746 | 31.19 | 1068.66 | 2137.32 | breast tumor | 1.86 |
| breast normal GW00-413 | 28798 | 37.01 | 32.76 | 32.76 | breast normal | |
| breast tumor GW00-412 | 28797 | 33.02 | 357.67 | 715.34 | breast tumor | 21.84 |
| breast normal GW00- | 27592-95 | 35.62 | 75.44 | 75.44 | breast normal | |
| 235:238 | | | | | | |
| breast tumor GW00- | 27588-91 | 31.32 | 989.78 | 989.78 | breast tumor | 13.12 |
| 231:234 | | | | | | |
| breast normal GW98-621 | 23656 | 28.72 | 4703.97 | 9407.94 | breast normal | |
| breast tumor GW98-620 | 23655 | 28.41 | 5654.66 | 11309.32 | breast tumor | 1.20 |
| brain normal BB99-542 | 25507 | 27.9 | 7676.77 | 15353.54 | brain normal | |
| brain normal BB99-406 | 25509 | 28.57 | 5145.99 | 10291.98 | brain normal | |
| brain normal BB99-904 | 25546 | 29.05 | 3866.67 | 7733.34 | brain normal | |
| brain stage 5 ALZ BB99- | 25502 | 28.31 | 6015.38 | 12030.76 | brain stage 5 ALZ | 1.08 |
| 874 | | | | | | |
| brain stage 5 ALZ BB99- | 25503 | 26.48 | 17975.27 | 35950.54 | brain stage 5 ALZ | 3.23 |
| 887 | | | | | | |
| brain stage 5 ALZ BB99- | 25504 | 27.66 | 8852.07 | 17704.14 | brain stage 5 ALZ | 1.59 |
| 862 | | | | | | |
| brain stage 5 ALZ BB99- | 25542 | 28.89 | 4235.74 | 8471.48 | brain stage 5 ALZ | −1.31 |
| 927 | | | | | | |
| CT lung | normal | 31.88 | 709.49 | 1418.98 | CT lung Nml | |
| lung 26 | normal | 24.63 | 54579.32 | | lung 26 Nml | |
| lung 27 | normal | 35.73 | 70.39 | 70.39 | lung 27 Nml | |
| lung 24 | COPD | 36.36 | 48.37 | 48.37 | lung 24 COPD | −12.03 |
| lung 28 | COPD | 32.44 | 506.79 | 506.79 | lung 28 COPD | −1.15 |
| lung 23 | COPD | 33.62 | 249.93 | 249.93 | lung 23 COPD | −2.33 |
| lung 25 | normal | 33.58 | 255.95 | 255.95 | lung 25 Nml | |
| asthmatic lung ODO3112 | 29321 | 35.26 | 93.39 | 93.39 | asthmatic lung | −6.23 |
| asthmatic lung ODO3433 | 29323 | 32.79 | 409.82 | 819.64 | asthmatic lung | 1.41 |
| asthmatic lung ODO3397 | 29322 | 33.36 | 291.41 | 582.82 | asthmatic lung | 1.00 |
| asthmatic lung ODO4928 | 29325 | 32.07 | 633.54 | 1267.08 | asthmatic lung | 2.18 |
| endo cells | control | 34.24 | 172.28 | 172.28 | endo cells | |
| endo VEGF | | 35.49 | 81.66 | 81.66 | endo VEGF | −2.11 |
| endo bFGF | | 34.74 | 128.01 | 128.01 | endo bFGF | −1.35 |
| heart Clontech | normal | 32.35 | 535.93 | 1071.86 | heart | |
| heart (T-1) ischemic | 29417 | 31.25 | 1030.24 | 2060.48 | heart (T-1) | 1.92 |
| | | | | | ischemic | |
| heart (T-14) non- | 29422 | 30.72 | 1417.06 | 2834.12 | heart (T-14) non- | 2.64 |
| obstructive DCM | | | | | obstructive DCM | |
| heart (T-3399) DCM | 29426 | 30.34 | 1778.61 | 3557.22 | heart (T-3399) | 3.32 |
| | | | | | DCM | |
| adenoid GW99-269 | 26162 | 30.66 | 1468.08 | 2936.16 | adenoid | |
| tonsil GW98-280 | 22582 | 30.26 | 1874.15 | 3748.30 | tonsil | |
| T cells PC00314 | 28453 | 38.46 | 13.71 | 27.42 | T cells | |
| PBMNC | | 40 | 0 | 0.00 | PBMNC | |
| monocyte | | 39.15 | 9.12 | 18.24 | monocyte | |
| B cells PC00665 | 28455 | 37.38 | 26.22 | 52.44 | B cells | |
| dendritic cells | 28441 | 33.71 | 237.32 | 474.64 | dendritic cells | |
| neutrophils | 28440 | 37.27 | 28.07 | 28.07 | neutrophils | |
| eosinophils | 28446 | 39.45 | 7.59 | 15.18 | eosinophils | |
| BM unstim | | 40 | 0 | 0.00 | BM unstim | |
| BM stim | treated | 40 | 0 | 0.00 | BM stim | 0.00 |
| osteo dif | treated | 31.2 | 1062.37 | 1062.37 | osteo dif | 2.11 |
| osteo undif | | 32.45 | 503.88 | 503.88 | osteo undif | |
| chondrocytes | | 30.75 | 1397.9 | 3494.75 | chondrocytes | |
| OA Synovium IP12/01 | 29462 | 29.3 | 3319.59 | 3319.59 | OA Synovium | |
| OA Synovium NP10/01 | 29461 | 30.09 | 2067.27 | 4134.54 | OA Synovium | |
| OA Synovium NP57/00 | 28464 | 29.57 | 2832.91 | 5665.82 | OA Synovium | |
| RA Synovium | 28466 | 29.37 | 3184.64 | 6369.28 | RA Synovium | |
| NP03/01 | | | | | | |
| RA Synovium | 28467 | 28.32 | 5987.67 | 11975.34 | RA Synovium | |
| NP71/00 | | | | | | |
| RA Synovium | 28475 | 28.28 | 6138.69 | 12277.38 | RA Synovium | |
| NP45/00 | | | | | | |
| OA bone (biobank) | 29217 | 32.72 | 428.55 | 428.55 | OA bone | |
| | | | | | (biobank) | |
| OA bone Sample 1 | J. Emory | 30.28 | 1850.45 | 3700.90 | OA bone | |
| OA bone Sample 2 | J. Emory | 31.23 | 1047.56 | 2095.12 | OA bone | |
| Cartilage (pool) | Normal | 28.39 | 5721.72 | 11443.44 | Nml Cartilage | |
| | | | | | (pool) | |
| Cartilage (pool) | OA | 29.25 | 3432.68 | 6865.36 | OA Cartilage | −1.67 |
| | | | | | (pool) | |
| PBL unifected | 28441 | 38.72 | 11.74 | 23.48 | PBL unifected | |
| PBL HIV IIIB | 28442 | 40 | 0 | 0.00 | PBL HIV IIIB | −23.48 |
| MRC5 uninfected (100%) | 29158 | 31.52 | 878.24 | 1756.48 | MRC5 | |
| | | | | | uninfected | |
| | | | | | (100%) | |
| MRC5 HSV strain F | 29178 | 37.92 | 19.03 | 38.06 | MRC5 HSV | −46.15 |
| | | | | | strain F | |
| W12 cells | 29179 | 36.14 | 55.05 | 110.10 | W12 cells | |
| Keratinocytes | 29180 | 35.36 | 88.09 | 176.18 | Keratinocytes | |
| B-actin control | | 25.47 | 32999.55 | | | |
| genomic | | 31.99 | 663.96 | | | |
| 1.00E+05 | | 22 | 100000 | | | |
| 1.00E+05 | | 22.24 | 100000 | | | |
| 1.00E+04 | | 27.59 | 10000 | | | |
| 1.00E+04 | | 27.53 | 10000 | | | |
| 1.00E+03 | | 32.07 | 1000 | | | |
| 1.00E+03 | | 32.22 | 1000 | | | |
| 1.00E+02 | | 39.16 | 100 | | | |
| 1.00E+02 | | 36.16 | 100 | | | |
| 1.00E+01 | | 39.45 | 10 | | | |
| 1.00E+01 | | 37.64 | 10 | | | |
| 1.00E+00 | | 40 | 0 | | | |
| 1.00E+00 | | 39.82 | 1 | | | |
| NTC | | 36.91 | −1 | | | |

*lung 26 Normal has been omitted due to multiple amplification failures from that sample

Gene Name sbg1558434SRCR

| | Fold Change in Disease |
|---|---|
| | Population Relative to |
| Disease tissues | Normal |
| colon tumor | 3.22 |
| colon tumor | −1.09 |
| colon tumor | 1.47 |
| colon tumor | −1.42 |
| lung tumor | 1.14 |
| lung tumor | −2.19 |
| lung tumor | 2.10 |
| lung tumor | −1.09 |
| breast tumor | 1.86 |
| breast tumor | 21.84 |
| breast tumor | 13.12 |
| breast tumor | 1.20 |
| brain stage 5 ALZ | 1.08 |
| brain stage 5 ALZ | 3.23 |
| brain stage 5 ALZ | 1.59 |
| brain stage 5 ALZ | −1.31 |
| lung 24 COPD | −12.03 |
| lung 28 COPD | −1.15 |
| lung 23 COPD | −2.33 |
| asthmatic lung | −6.23 |
| asthmatic lung | 1.41 |
| asthmatic lung | 1.00 |
| asthmatic lung | 2.18 |
| endo VEGF | −2.11 |
| endo bFGF | −1.35 |
| heart (T-1) ischemic | 1.92 |
| heart (T-14) non-obstructive DCM | 2.64 |
| heart (T-3399) DCM | 3.32 |
| BM stim | 0.00 |
| osteo dif | 2.11 |
| OA Cartilage (pool) | −1.67 |
| PBL HIV IIIB | −23.48 |
| MRC5 HSV strain F | −46.15 |

Gene Name sbg1546354LRR

This primer set was shown to amplify two products of approximately equal proportions by the dissociation curve program. Moderate to low overall expression. Highest normal expression is seen in the whole brain, endometrium, and uterus. Good levels of expression are seen in all of the samples representing the female reproductive system. Highest disease expression is seen in the normal and Alzheimer's brain samples. Downregulation in 2 of 4 lung tumors and upregulation in 3 of 4 breast tumors implicates this gene in cancers of the lung and breast. Downregulation in 1 of 4 Alzheimer's brain samples may suggest a role in Alzheimer's disease. Downregulation in 3 of 3 COPD samples and in 3 of 4 asthmatic lung samples suggests a potential role for this gene in chronic obstructive pulmonary disorder and asthma. Upregulation in 3 of 3 disease heart samples implies an involvement in cardiovascular diseases such as non-obstructive and obstructive DCM and ischemia. Upregulation in the OA cartilage sample suggests a possible role for this gene in osteoarthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample 1 | (sample | (sample | GOI | rRNA | rRNA | total |
| sbg1546354LRR | and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous | 40, 40 | 0.45 | 0.39 | 0.42 | 0.96 | 52.36 | 21.99 |
| Adipose Zenbio | | | | | | | |
| Adrenal Gland | 38.55, 39.05 | 1.15 | 0.88 | 1.02 | 0.61 | 81.97 | 83.20 |
| Clontech | | | | | | | |
| Whole Brain | 22.45, 22.92 | 7165.24 | 5554.84 | 6360.04 | 7.24 | 6.91 | 43922.93 |
| Clontech | | | | | | | |
| Fetal Brain Clontech | 40, 39.61 | 0.46 | 0.65 | 0.56 | 0.48 | 103.95 | 57.69 |
| Cerebellum Clontech | 40, 34.21 | 0 | 12.15 | 6.08 | 2.17 | 23.04 | 139.98 |
| Cervix | 40, 36.23 | 0 | 4.05 | 2.03 | 2.42 | 20.66 | 41.84 |
| Colon | 40, 40 | 0.39 | 0 | 0.20 | 2.71 | 18.45 | 3.60 |
| Endometrium | 34.32, 32.75 | 11.42 | 26.84 | 19.13 | 0.73 | 68.21 | 1304.91 |
| Esophagus | 37.29, 37.76 | 2.28 | 1.77 | 2.03 | 1.37 | 36.50 | 73.91 |
| Heart Clontech | 39.67, 38.45 | 0.63 | 1.21 | 0.92 | 1.32 | 37.88 | 34.85 |
| Hypothalamus | 40, 40 | 0 | 0 | 0.00 | 0.32 | 155.28 | 0.00 |
| Ileum | 37.74, 38.72 | 1.78 | 1.05 | 1.42 | 2.58 | 19.38 | 27.42 |
| Jejunum | 34.06, 34.2 | 13.18 | 12.16 | 12.67 | 6.60 | 7.58 | 95.98 |
| Kidney | 39.29, 40 | 0.77 | 0 | 0.39 | 2.12 | 23.58 | 9.08 |
| Liver | 34, 37.64 | 13.6 | 1.89 | 7.75 | 1.50 | 33.33 | 258.17 |
| Fetal Liver Clontech | 39.07, 36.21 | 0.87 | 4.1 | 2.49 | 10.40 | 4.81 | 11.95 |
| Lung | 39.45, 39.92 | 0.71 | 0.57 | 0.64 | 2.57 | 19.46 | 12.45 |
| Mammary Gland | 38.43, 35.9 | 1.23 | 4.85 | 3.04 | 13.00 | 3.85 | 11.69 |
| Clontech | | | | | | | |
| Myometrium | 38.88, 34.92 | 0.96 | 8.24 | 4.60 | 2.34 | 21.37 | 98.29 |
| Omentum | 37.96, 38.78 | 1.58 | 1.02 | 1.30 | 3.94 | 12.69 | 16.50 |
| Ovary | 33.4, 32.97 | 18.8 | 23.7 | 21.25 | 4.34 | 11.52 | 244.82 |
| Pancreas | 40, 39.83 | 0 | 0.58 | 0.29 | 0.81 | 61.80 | 17.92 |
| Head of Pancreas | 40, 40 | 0.5 | 0 | 0.25 | 1.57 | 31.85 | 7.96 |
| Parotid Gland | 37.46, 33.84 | 2.08 | 14.78 | 8.43 | 5.48 | 9.12 | 76.92 |
| Placenta Clontech | 36.58, 33.33 | 3.35 | 19.54 | 11.45 | 5.26 | 9.51 | 108.79 |
| Prostate | 39.19, 40 | 0.81 | 0.58 | 0.70 | 3.00 | 16.67 | 11.58 |
| Rectum | 40, 37.44 | 0 | 2.11 | 1.06 | 1.23 | 40.65 | 42.89 |
| Salivary Gland | 38.17, 40 | 1.41 | 0.42 | 0.92 | 7.31 | 6.84 | 6.26 |
| Clontech | | | | | | | |
| Skeletal Muscle | 37.17, 40 | 2.43 | 0 | 1.22 | 1.26 | 39.68 | 48.21 |
| Clontech | | | | | | | |
| Skin | 39.11, 39.87 | 0.85 | 0.56 | 0.71 | 1.21 | 41.32 | 29.13 |
| Small Intestine | 39.14, 38.66 | 0.83 | 1.08 | 0.96 | 0.98 | 51.07 | 48.77 |
| Clontech | | | | | | | |
| Spleen | 38.75, 35.13 | 1.03 | 7.35 | 4.19 | 4.92 | 10.16 | 42.58 |
| Stomach | 39.38, 39.17 | 0.73 | 0.82 | 0.78 | 2.73 | 18.32 | 14.19 |
| Testis Clontech | 40, 39.46 | 0 | 0.7 | 0.35 | 0.57 | 87.87 | 30.76 |
| Thymus Clontech | 31.76, 31.99 | 45.85 | 40.36 | 43.11 | 9.89 | 5.06 | 217.92 |
| Thyroid | 37.48, 33.8 | 2.06 | 15.17 | 8.62 | 2.77 | 18.05 | 155.51 |
| Trachea Clontech | 34.12, 34.14 | 12.73 | 12.58 | 12.66 | 9.71 | 5.15 | 65.16 |
| Urinary Bladder | 33.97, 38.52 | 13.82 | 1.17 | 7.50 | 5.47 | 9.14 | 68.51 |
| Uterus | 30.75, 31.24 | 79.28 | 60.67 | 69.98 | 5.34 | 9.36 | 655.20 |
| genomic | 25.24 | 1579.11 | | | | | |
| b-actin | 26 | 1042.55 | | | | | |
| 1.00E+05 | 18 | 100000 | | | | | |
| 1.00E+05 | 18.21 | 100000 | | | | | |
| 1.00E+04 | 21.29 | 10000 | | | | | |
| 1.00E+04 | 21.33 | 10000 | | | | | |
| 1.00E+03 | 25.09 | 1000 | | | | | |
| 1.00E+03 | 24.96 | 1000 | | | | | |
| 1.00E+02 | 32.46 | 100 | | | | | |
| 1.00E+02 | 29.76 | 100 | | | | | |
| 1.00E+01 | 33.68 | 10 | | | | | |
| 1.00E+01 | 37.73 | 10 | | | | | |
| 1.00E+00 | 38.01 | 1 | | | | | |
| 1.00E+00 | 37.89 | 1 | | | | | |
| NTC | 40 | 0 | | | | | |
| NTC | 39.22 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1546354LRR | identifier) | Ct | copies | RNA | Sample | Population |
| colon normal GW98-167 | 21941 | 32.92 | 25.83 | 51.66 | colon normal | |
| colon tumor GW98-166 | 21940 | 33.26 | 21.39 | 42.78 | colon tumor | −1.21 |
| colon normal GW98-178 | 22080 | 32.74 | 28.67 | 57.34 | colon normal | |
| colon tumor GW98-177 | 22060 | 35.12 | 7.61 | 15.22 | colon tumor | −3.77 |
| colon normal GW98-561 | 23514 | 32.71 | 29.09 | 58.18 | colon normal | |
| colon tumor GW98-560 | 23513 | 33.09 | 23.59 | 47.18 | colon tumor | −1.23 |
| colon normal GW98-894 | 24691 | 32.44 | 33.79 | 67.58 | colon normal | |
| colon tumor GW98-893 | 24690 | 32.14 | 40.07 | 80.14 | colon tumor | 1.19 |
| lung normal GW98-3 | 20742 | 32.37 | 35.14 | 70.28 | lung normal | |
| lung tumor GW98-2 | 20741 | 33.74 | 16.37 | 32.74 | lung tumor | −2.15 |
| lung normal GW97-179 | 20677 | 30.45 | 102.87 | 205.74 | lung normal | |
| lung tumor GW97-178 | 20676 | 34.06 | 13.68 | 27.36 | lung tumor | −7.52 |
| lung normal GW98-165 | 21922 | 30.69 | 89.75 | 179.50 | lung normal | |
| lung tumor GW98-164 | 21921 | 28.52 | 300.37 | 600.74 | lung tumor | 3.35 |
| lung normal GW98-282 | 22584 | 30.49 | 100.24 | 200.48 | lung normal | |
| lung tumor GW98-281 | 22583 | 34.72 | 9.48 | 18.96 | lung tumor | −10.57 |
| breast normal GW00-392 | 28750 | 31.86 | 46.72 | 46.72 | breast normal | |
| breast tumor GW00-391 | 28746 | 29.77 | 150.15 | 300.30 | breast tumor | 6.43 |
| breast normal GW00-413 | 28798 | 39.07 | 0.84 | 0.84 | breast normal | |
| breast tumor GW00-412 | 28797 | 34.47 | 10.94 | 21.88 | breast tumor | 26.05 |
| breast normal GW00- | 27592-95 | 38.66 | 1.05 | 1.05 | breast normal | |
| 235:238 | | | | | | |
| breast tumor GW00- | 27588-91 | 34.58 | 10.25 | 10.25 | breast tumor | 9.76 |
| 231:234 | | | | | | |
| breast normal GW98-621 | 23656 | 34.45 | 11.01 | 22.02 | breast normal | |
| breast tumor GW98-620 | 23655 | 35.28 | 6.93 | 13.86 | breast tumor | −1.59 |
| brain normal BB99-542 | 25507 | 24.64 | 2625.08 | 5250.16 | brain normal | |
| brain normal BB99-406 | 25509 | 27.93 | 418.16 | 836.32 | brain normal | |
| brain normal BB99-904 | 25546 | 26.28 | 1048.78 | 2097.56 | brain normal | |
| brain stage 5 ALZ BB99- | 25502 | 29.47 | 177.72 | 355.44 | brain stage 5 ALZ | −7.68 |
| 874 | | | | | | |
| brain stage 5 ALZ BB99- | 25503 | 25.5 | 1620.02 | 3240.04 | brain stage 5 ALZ | 1.19 |
| 887 | | | | | | |
| brain stage 5 ALZ BB99- | 25504 | 25.96 | 1251.61 | 2503.22 | brain stage 5 ALZ | −1.09 |
| 862 | | | | | | |
| brain stage 5 ALZ BB99- | 25542 | 25.83 | 1348.09 | 2696.18 | brain stage 5 ALZ | −1.01 |
| 927 | | | | | | |
| CT lung | normal | 32.74 | 28.6 | 57.20 | CT lung Nml | |
| lung 26 | normal | 29.2 | 205.78 | | lung 26 Nml | |
| lung 27 | normal | 40 | 0 | 0.00 | lung 27 Nml | |
| lung 24 | COPD | 40 | 0 | 0.00 | lung 24 COPD | −19.69 |
| lung 28 | COPD | 40 | 0 | 0.00 | lung 28 COPD | −19.69 |
| lung 23 | COPD | 40 | 0 | 0.00 | lung 23 COPD | −19.69 |
| lung 25 | normal | 37.63 | 1.87 | 1.87 | lung 25 Nml | |
| asthmatic lung ODO3112 | 29321 | 37.11 | 2.5 | 2.50 | asthmatic lung | −7.88 |
| asthmatic lung ODO3433 | 29323 | 35.13 | 7.54 | 15.08 | asthmatic lung | −1.31 |
| asthmatic lung ODO3397 | 29322 | 36.76 | 3.03 | 6.06 | asthmatic lung | −3.25 |
| asthmatic lung ODO4928 | 29325 | 37.12 | 2.48 | 4.96 | asthmatic lung | −3.97 |
| endo cells | control | 35.13 | 7.56 | 7.56 | endo cells | |
| endo VEGF | | 36.92 | 2.79 | 2.79 | endo VEGF | −2.71 |
| endo bFGF | | 34.52 | 10.6 | 10.60 | endo bFGF | 1.40 |
| heart Clontech | normal | 37.63 | 1.87 | 3.74 | heart | |
| heart (T-1) ischemic | 29417 | 35.2 | 7.28 | 14.56 | heart (T-1) | 3.89 |
| | | | | | ischemic | |
| heart (T-14) non- | 29422 | 33.92 | 14.83 | 29.66 | heart (T-14) non- | 7.93 |
| obstructive DCM | | | | | obstructive DCM | |
| heart (T-3399) DCM | 29426 | 33.91 | 14.89 | 29.78 | heart (T-3399) | 7.96 |
| | | | | | DCM | |
| adenoid GW99-269 | 26162 | 36.84 | 2.91 | 5.82 | adenoid | |
| tonsil GW98-280 | 22582 | 38.06 | 1.47 | 2.94 | tonsil | |
| T cells PC00314 | 28453 | 34.48 | 10.87 | 21.74 | T cells | |
| PBMNC | | 40 | 0 | 0.00 | PBMNC | |
| monocyte | | 40 | 0 | 0.00 | monocyte | |
| B cells PC00665 | 28455 | 37.71 | 1.8 | 3.60 | B cells | |
| dendritic cells | 28441 | 34.1 | 13.44 | 26.88 | dendritic cells | |
| neutrophils | 28440 | 33.53 | 18.38 | 18.38 | neutrophils | |
| eosinophils | 28446 | 36.39 | 3.75 | 7.50 | eosinophils | |
| BM unstim | | 40 | 0 | 0.00 | BM unstim | |
| BM stim | treated | 37.26 | 2.31 | 2.31 | BM stim | 2.31 |
| osteo dif | treated | 39.27 | 0.75 | 0.75 | osteo dif | 0.75 |
| osteo undif | | 40 | 0 | 0.00 | osteo undif | |
| chondrocytes | | 35.46 | 6.27 | 15.68 | chondrocytes | |
| OA Synovium IP12/01 | 29462 | 34.8 | 9.09 | 9.09 | OA Synovium | |
| OA Synovium NP10/01 | 29461 | 37.51 | 2 | 4.00 | OA Synovium | |
| OA Synovium NP57/00 | 28464 | 31.86 | 46.85 | 93.70 | OA Synovium | |
| RA Synovium | 28466 | 32.26 | 37.32 | 74.64 | RA Synovium | |
| NP03/01 | | | | | | |
| RA Synovium | 28467 | 33.22 | 21.88 | 43.76 | RA Synovium | |
| NP71/00 | | | | | | |
| RA Synovium | 28475 | 31.52 | 56.4 | 112.80 | RA Synovium | |
| NP45/00 | | | | | | |
| OA bone (biobank) | 29217 | 40 | 0 | 0.00 | OA bone (biobank) | |
| OA bone Sample 1 | J. Emory | 39.56 | 0.64 | 1.28 | OA bone | |
| OA bone Sample 2 | J. Emory | 36.53 | 3.46 | 6.92 | OA bone | |
| Cartilage (pool) | Normal | 38.31 | 1.28 | 2.56 | Nml Cartilage | |
| | | | | | (pool) | |
| Cartilage (pool) | OA | 33.77 | 16.11 | 32.22 | OA Cartilage | 12.59 |
| | | | | | (pool) | |
| PBL unifected | 28441 | 34.27 | 12.18 | 24.36 | PBL unifected | |
| PBL HIV IIIB | 28442 | 34.68 | 9.69 | 19.38 | PBL HIV IIIB | −1.26 |
| MRC5 uninfected (100%) | 29158 | 35.57 | 5.9 | 11.80 | MRC5 uninfected | |
| | | | | | (100%) | |
| MRC5 HSV strain F | 29178 | 38.1 | 1.44 | 2.88 | MRC5 HSV strain F | −4.10 |
| W12 cells | 29179 | 36.18 | 4.2 | 8.40 | W12 cells | |
| Keratinocytes | 29180 | 32.92 | 25.89 | 51.78 | Keratinocytes | |
| B-actin control | | 26.2 | 1097.83 | | | |
| genomic | | 25.78 | 1386.79 | | | |
| 1.00E+05 | | 18.35 | 100000 | | | |
| 1.00E+05 | | 18.44 | 100000 | | | |
| 1.00E+04 | | 21.88 | 10000 | | | |
| 1.00E+04 | | 21.87 | 10000 | | | |
| 1.00E+03 | | 25.52 | 1000 | | | |
| 1.00E+03 | | 26.11 | 1000 | | | |
| 1.00E+02 | | 33.25 | 100 | | | |
| 1.00E+02 | | 29.64 | 100 | | | |
| 1.00E+01 | | 39.97 | 10 | | | |
| 1.00E+01 | | 34.19 | 10 | | | |
| 1.00E+00 | | 38.21 | 1 | | | |
| 1.00E+00 | | 39.11 | 1 | | | |
| NTC | | 38.2 | −1 | | | |

*lung 26 Normal has been omitted due to multiple amplification failures from that sample

Gene Name sbg1546354LRR

| | Fold Change in Disease |
|---|---|
| | Population Relative to |
| Disease tissues | Normal |
| colon tumor | −1.21 |
| colon tumor | −3.77 |
| colon tumor | −1.23 |
| colon tumor | 1.19 |
| lung tumor | −2.15 |
| lung tumor | −7.52 |
| lung tumor | 3.35 |
| lung tumor | −10.57 |
| breast tumor | 6.43 |
| breast tumor | 26.05 |
| breast tumor | 9.76 |
| breast tumor | −1.59 |
| brain stage 5 ALZ | −7.68 |
| brain stage 5 ALZ | 1.19 |
| brain stage 5 ALZ | −1.09 |
| brain stage 5 ALZ | −1.01 |
| lung 24 COPD | −19.69 |
| lung 28 COPD | −19.69 |
| lung 23 COPD | −19.69 |
| asthmatic lung | −7.88 |
| asthmatic lung | −1.31 |
| asthmatic lung | −3.25 |
| asthmatic lung | −3.97 |
| endo VEGF | −2.71 |
| endo bFGF | 1.40 |
| heart (T-1) ischemic | 3.89 |
| heart (T-14) non-obstructive DCM | 7.93 |
| heart (T-3399) DCM | 7.96 |
| BM stim | 2.31 |
| osteo dif | 0.75 |
| OA Cartilage (pool) | 12.59 |
| PBL HIV IIIB | −1.26 |
| MRC5 HSV strain F | −4.10 |

Gene Name sbg1555869CABP

Failed.

Gene Name sbg1518024NKR

Moderate to low overall expression. The highest normal expression is seen in the liver with lower levels of expression in the tissues specific to the female reproductive system including the uterus, cervix, endometrium, myometrium, and the ovary. The highest disease expression is seen in the HIV-infected and uninfected peripheral blood lymphocytes. Upregulation in 2 of 4 colon tumor samples implicates this gene in colon cancer. Downregulation in 2 of 4 lung tumor samples suggests a potential role in cancer of the lung. Upregulation in 1 of 4 breast tumor samples implicates this gene in breast cancer. Upregulation in the VEGF-treated endothelial cell line implicates a possible role for this gene in angiogenesis. Upregulation in the HSV-infected MRC5 cells suggests that this gene may be a host factor in HSV. Moderate to high expression in the RA and OA synovium samples, the OA bone samples, and the chondrocytes with corroborating high expression in the PBMNCs, B cells, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1518024NKR | 1 and 2) | 1) | 2) | Copies | (ng) | (ng) | RNA |
| Subcutaneous | 40, 37.18 | 0 | 0.53 | 0.27 | 3.06 | 16.34 | 4.33 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 39.59 | 0 | 0.12 | 0.06 | 0.61 | 81.97 | 4.92 |
| Whole Brain Clontech | 34.25, 37.04 | 3.37 | 0.58 | 1.98 | 7.24 | 6.91 | 13.64 |
| Fetal Brain Clontech | 39.3, 40 | 0.14 | 0 | 0.07 | 0.48 | 103.95 | 7.28 |
| Cerebellum Clontech | 35, 40 | 2.1 | 0 | 1.05 | 2.17 | 23.04 | 24.19 |
| Cervix | 33.92, 35.2 | 4.13 | 1.84 | 2.99 | 2.42 | 20.66 | 61.67 |
| Colon | 40, 37.27 | 0 | 0.5 | 0.25 | 2.71 | 18.45 | 4.61 |
| Endometrium | 37.16, 34.98 | 0.54 | 2.12 | 1.33 | 0.73 | 68.21 | 90.72 |
| Esophagus | 39.19, 40 | 0.15 | 0 | 0.08 | 1.37 | 36.50 | 2.74 |
| Heart Clontech | 40, 40 | 0 | 0 | 0.00 | 1.32 | 37.88 | 0.00 |
| Hypothalamus | 40, 39.13 | 0 | 0.15 | 0.08 | 0.32 | 155.28 | 11.65 |
| Ileum | 40, 37.58 | 0 | 0.41 | 0.21 | 2.58 | 19.38 | 3.97 |
| Jejunum | 34.43, 33.67 | 3 | 4.84 | 3.92 | 6.60 | 7.58 | 29.70 |
| Kidney | 40, 37.82 | 0 | 0.35 | 0.18 | 2.12 | 23.58 | 4.13 |
| Liver | 32.48, 32.79 | 10.26 | 8.41 | 9.34 | 1.50 | 33.33 | 311.17 |
| Fetal Liver Clontech | 35.88, 40 | 1.2 | 0 | 0.60 | 10.40 | 4.81 | 2.88 |
| Lung | 34.3, 35.15 | 3.26 | 1.9 | 2.58 | 2.57 | 19.46 | 50.19 |
| Mammary Gland | 37.99, 35.57 | 0.32 | 1.46 | 0.89 | 13.00 | 3.85 | 3.42 |
| Clontech | | | | | | | |
| Myometrium | 36.21, 35.15 | 0.98 | 1.9 | 1.44 | 2.34 | 21.37 | 30.77 |
| Omentum | 40, 34.97 | 0 | 2.13 | 1.07 | 3.94 | 12.69 | 13.52 |
| Ovary | 33.51, 34.04 | 5.37 | 3.84 | 4.61 | 4.34 | 11.52 | 53.05 |
| Pancreas | 36.13, 36.05 | 1.03 | 1.08 | 1.06 | 0.81 | 61.80 | 65.20 |
| Head of Pancreas | 40, 40 | 0 | 0 | 0.00 | 1.57 | 31.85 | 0.00 |
| Parotid Gland | 34.91, 39.48 | 2.22 | 0.12 | 1.17 | 5.48 | 9.12 | 10.68 |
| Placenta Clontech | 40, 35.62 | 0 | 1.42 | 0.71 | 5.26 | 9.51 | 6.75 |
| Prostate | 36.16, 37.79 | 1.01 | 0.36 | 0.69 | 3.00 | 16.67 | 11.42 |
| Rectum | 40, 38.6 | 0 | 0.22 | 0.11 | 1.23 | 40.65 | 4.47 |
| Salivary Gland Clontech | 37.14, 34.66 | 0.54 | 2.59 | 1.57 | 7.31 | 6.84 | 10.70 |
| Skeletal Muscle | 40, 40 | 0 | 0 | 0.00 | 1.26 | 39.68 | 0.00 |
| Clontech | | | | | | | |
| Skin | 38.65, 34.71 | 0.21 | 2.51 | 1.36 | 1.21 | 41.32 | 56.20 |
| Small Intestine Clontech | 40, 40 | 0 | 0 | 0.00 | 0.98 | 51.07 | 0.00 |
| Spleen | 36.41, 38.53 | 0.86 | 0.23 | 0.55 | 4.92 | 10.16 | 5.54 |
| Stomach | 36.03, 34.11 | 1.09 | 3.67 | 2.38 | 2.73 | 18.32 | 43.59 |
| Testis Clontech | 40, 40 | 0 | 0 | 0.00 | 0.57 | 87.87 | 0.00 |
| Thymus Clontech | 33.89, 35.15 | 4.2 | 1.9 | 3.05 | 9.89 | 5.06 | 15.42 |
| Thyroid | 36.33, 40 | 0.9 | 0 | 0.45 | 2.77 | 18.05 | 8.12 |
| Trachea Clontech | 38.43, 36.09 | 0.24 | 1.05 | 0.65 | 9.71 | 5.15 | 3.32 |
| Urinary Bladder | 40, 37.25 | 0 | 0.51 | 0.26 | 5.47 | 9.14 | 2.33 |
| Uterus | 31.93, 32.48 | 14.54 | 10.24 | 12.39 | 5.34 | 9.36 | 116.01 |
| genomic | 24.9 | 1225.76 | | | | | |
| b-actin | 27.36 | 258.77 | | | | | |
| 1.00E+05 | 18.17 | 100000 | | | | | |
| 1.00E+05 | 18.21 | 100000 | | | | | |
| 1.00E+04 | 21.27 | 10000 | | | | | |
| 1.00E+04 | 21.19 | 10000 | | | | | |
| 1.00E+03 | 24.74 | 1000 | | | | | |
| 1.00E+03 | 24.69 | 1000 | | | | | |
| 1.00E+02 | 29.57 | 100 | | | | | |
| 1.00E+02 | 29.1 | 100 | | | | | |
| 1.00E+01 | 32.13 | 10 | | | | | |
| 1.00E+01 | 34.09 | 10 | | | | | |
| 1.00E+00 | 40 | 0 | | | | | |
| 1.00E+00 | 35.2 | 1 | | | | | |
| NTC | 40 | 0 | | | | | |
| NTC | 40 | 0 | | | | | |

| | | | | copies | | |
|---|---|---|---|---|---|---|
| | | | | of mRNA | | Fold |
| | Reg | | | detected / | | Change |
| | number | | Mean | 50 ng | | in |
| Sample | (GSK | | GOI | total | | Disease |
| sbg1518024NKR | identifier ) | Ct | copies | RNA | Sample | Population |
| colon normal GW98-167 | 21941 | 30.98 | 26.90 | 53.80 | colon normal | |
| colon tumor GW98-166 | 21940 | 33.55 | 7.50 | 14.99 | colon tumor | −3.59 |
| colon normal GW98-178 | 22080 | 37.16 | 1.46 | 2.91 | colon normal | |
| colon tumor GW98-177 | 22060 | 35.29 | 3.33 | 6.67 | colon tumor | 2.29 |
| colon normal GW98-561 | 23514 | 39.1 | 0.64 | 1.29 | colon normal | |
| colon tumor GW98-560 | 23513 | 33.49 | 7.71 | 15.43 | colon tumor | 11.98 |
| colon normal GW98-894 | 24691 | 35.69 | 2.78 | 5.56 | colon normal | |
| colon tumor GW98-893 | 24690 | 32.33 | 13.58 | 27.15 | colon tumor | 4.88 |
| lung normal GW98-3 | 20742 | 30.32 | 37.99 | 75.98 | lung normal | |
| lung tumor GW98-2 | 20741 | 34.37 | 5.09 | 10.18 | lung tumor | −7.46 |
| lung normal GW97-179 | 20677 | 32.59 | 11.94 | 23.88 | lung normal | |
| lung tumor GW97-178 | 20676 | 31.04 | 26.08 | 52.16 | lung tumor | 2.18 |
| lung normal GW98-165 | 21922 | 29.1 | 73.39 | 146.78 | lung normal | |
| lung tumor GW98-164 | 21921 | 34.05 | 5.91 | 11.83 | lung tumor | −12.41 |
| lung normal GW98-282 | 22584 | 34.42 | 4.97 | 9.95 | lung normal | |
| lung tumor GW98-281 | 22583 | 32.16 | 14.77 | 29.55 | lung tumor | 2.97 |
| breast normal GW00-392 | 28750 | 32.66 | 11.54 | 11.54 | breast normal | |
| breast tumor GW00-391 | 28746 | 38.04 | 1.00 | 2.00 | breast tumor | −5.76 |
| breast normal GW00-413 | 28798 | 35.03 | 3.75 | 3.75 | breast normal | |
| breast tumor GW00-412 | 28797 | 30.75 | 30.31 | 60.63 | breast tumor | 16.16 |
| breast normal GW00- | 27592-95 | 37.22 | 1.42 | 1.42 | breast normal | |
| 235:238 | | | | | | |
| breast tumor GW00- | 27588-91 | 36.24 | 2.18 | 2.18 | breast tumor | 1.53 |
| 231:234 | | | | | | |
| breast normal GW98-621 | 23656 | 34.34 | 5.16 | 10.32 | breast normal | |
| breast tumor GW98-620 | 23655 | 34.52 | 4.75 | 9.49 | breast tumor | −1.09 |
| brain normal BB99-542 | 25507 | 34.07 | 5.86 | 11.72 | brain normal | |
| brain normal BB99-406 | 25509 | 40 | 0.45 | 0.89 | brain normal | |
| brain normal BB99-904 | 25546 | 36.33 | 2.09 | 4.18 | brain normal | |
| brain stage 5 ALZ BB99- | 25502 | 35.45 | 3.10 | 6.20 | brain stage 5 ALZ | 1.11 |
| 874 | | | | | | |
| brain stage 5 ALZ BB99- | 25503 | 35.07 | 3.68 | 7.37 | brain stage 5 ALZ | 1.32 |
| 887 | | | | | | |
| brain stage 5 ALZ BB99- | 25504 | 35.35 | 3.24 | 6.49 | brain stage 5 ALZ | 1.16 |
| 862 | | | | | | |
| brain stage 5 ALZ BB99- | 25542 | 39.36 | 0.58 | 1.16 | brain stage 5 ALZ | −4.83 |
| 927 | | | | | | |
| CT lung | normal | 31.4 | 21.68 | 43.35 | CT lung Nml | |
| lung 26 | normal | 33.97 | 6.14 | | lung 26 Nml | |
| lung 27 | normal | 33.88 | 6.41 | 6.41 | lung 27 Nml | |
| lung 24 | COPD | 35.94 | 2.49 | 2.49 | lung 24 COPD | −7.20 |
| lung 28 | COPD | 35.07 | 3.68 | 3.68 | lung 28 COPD | −4.86 |
| lung 23 | COPD | 38.79 | 0.73 | 0.73 | lung 23 COPD | −24.46 |
| lung 25 | normal | 34.92 | 3.95 | 3.95 | lung 25 Nml | |
| asthmatic lung ODO3112 | 29321 | 32.7 | 11.31 | 11.31 | asthmatic lung | −1.58 |
| asthmatic lung ODO3433 | 29323 | 31.02 | 26.35 | 52.70 | asthmatic lung | 2.94 |
| asthmatic lung ODO3397 | 29322 | 32.05 | 15.61 | 31.21 | asthmatic lung | 1.74 |
| asthmatic lung ODO4928 | 29325 | 33.14 | 9.13 | 18.26 | asthmatic lung | 1.02 |
| endo cells | control | 40 | 0.45 | 0.45 | endo cells | |
| endo VEGF | | 35.32 | 3.29 | 3.29 | endo VEGF | 7.35 |
| endo bFGF | | 40 | 0.45 | 0.45 | endo bFGF | 1.00 |
| heart Clontech | normal | 33.28 | 8.53 | 17.07 | heart | |
| heart (T-1) ischemic | 29417 | 34.16 | 5.62 | 11.23 | heart (T-1) | −1.52 |
| | | | | | ischemic | |
| heart (T-14) non- | 29422 | 33.01 | 9.72 | 19.45 | heart (T-14) non- | 1.14 |
| obstructive DCM | | | | | obstructive DCM | |
| heart (T-3399) DCM | 29426 | 34.08 | 5.83 | 11.66 | heart (T-3399) | −1.46 |
| | | | | | DCM | |
| adenoid GW99-269 | 26162 | 40 | 0.45 | 0.89 | adenoid | |
| tonsil GW98-280 | 22582 | 33.34 | 8.29 | 16.58 | tonsil | |
| T cells PC00314 | 28453 | 34.18 | 5.56 | 11.13 | T cells | |
| PBMNC | | 33.86 | 6.47 | 6.47 | PBMNC | |
| monocyte | | 34.74 | 4.29 | 8.57 | monocyte | |
| B cells PC00665 | 28455 | 32.36 | 13.38 | 26.75 | B cells | |
| dendritic cells | 28441 | 40 | 0.45 | 0.89 | dendritic cells | |
| neutrophils | 28440 | 30.85 | 28.78 | 28.78 | neutrophils | |
| eosinophils | 28446 | 39.24 | 0.61 | 1.22 | eosinophils | |
| BM unstim | | 32.98 | 9.87 | 9.87 | BM unstim | |
| BM stim | treated | 30.62 | 32.44 | 32.44 | BM stim | 3.29 |
| osteo dif | treated | 40 | 0.45 | 0.45 | osteo dif | 1.00 |
| osteo undif | | 40 | 0.45 | 0.45 | osteo undif | |
| chondrocytes | | 35.26 | 3.38 | 8.45 | chondrocytes | |
| OA Synovium IP12/01 | 29462 | 34.36 | 5.11 | 5.11 | OA Synovium | |
| OA Synovium NP10/01 | 29461 | 37.48 | 1.27 | 2.54 | OA Synovium | |
| OA Synovium NP57/00 | 28464 | 33.51 | 7.64 | 15.28 | OA Synovium | |
| RA Synovium | 28466 | 33.5 | 7.68 | 15.36 | RA Synovium | |
| NP03/01 | | | | | | |
| RA Synovium | 28467 | 30.54 | 33.83 | 67.67 | RA Synovium | |
| NP71/00 | | | | | | |
| RA Synovium ___{—} | 28475 | 34.1 | 5.78 | 11.55 | RA Synovium | |
| NP45/00 | | | | | | |
| OA bone (biobank) | 29217 | 32.39 | 13.18 | 13.18 | OA bone (biobank) | |
| OA bone Sample 1 | J. Emory | 34.31 | 5.23 | 10.47 | OA bone | |
| OA bone Sample 2 | J. Emory | 33.15 | 9.09 | 18.17 | OA bone | |
| Cartilage (pool) | Normal | 33.56 | 7.46 | 14.92 | Nml Cartilage | |
| | | | | | (pool) | |
| Cartilage (pool) | OA | 34.94 | 3.91 | 7.82 | OA Cartilage | −1.91 |
| | | | | | (pool) | |
| PBL unifected | 28441 | 27.6 | 171.44 | 342.88 | PBL unifected | |
| PBL HIV IIIB | 28442 | 26.15 | 407.26 | 814.52 | PBL HIV IIIB | 2.38 |
| MRC5 uninfected | 29158 | 40 | 0.45 | 0.89 | MRC5 uninfected | |
| (100%) | | | | | (100%) | |
| MRC5 HSV strain F | 29178 | 35.02 | 3.77 | 7.54 | MRC5 HSV strain F | 8.43 |
| W12 cells | 29179 | 40 | 0.45 | 0.89 | W12 cells | 1.00 |
| Keratinocytes | 29180 | 40 | 0.45 | 0.89 | Keratinocytes | |
| B-actin control | | 27.29 | 205.48 | | | |
| genomic | | 24.73 | 996.82 | | Disease plate did not have | |
| | | | | | genomic samples. | |
| 1.00E+05 | | 40 | 0.45 | | Copy number was, therefore | |
| | | | | | calculated from the gene- | |
| | | | | | specific genomic curve on | |
| | | | | | the normal plate. | |
| 1.00E+05 | | 40 | 0.45 | | | |
| 1.00E+04 | | 39.72 | 0.50 | | | |
| 1.00E+04 | | 40 | 0.45 | | | |
| 1.00E+03 | | 40 | 0.45 | | | |
| 1.00E+03 | | 40 | 0.45 | | | |
| 1.00E+02 | | 38.14 | 0.96 | | | |
| 1.00E+02 | | 40 | 0.45 | | | |
| 1.00E+01 | | 40 | 0.45 | | | |
| 1.00E+01 | | 40 | 0.45 | | | |
| 1.00E+00 | | 40 | 0.45 | | | |
| 1.00E+00 | | 38.5 | 0.83 | | | |
| NTC | | 40 | 0.45 | | | |

*lung 26 Normal has been omitted due to multiple amplification failures from that sample

Gene Name sbg1518024NKR

| | Fold Change in Disease |
|---|---|
| | Population Relative to |
| Disease tissues | Normal |
| colon tumor | −3.59 |
| colon tumor | 2.29 |
| colon tumor | 11.98 |
| colon tumor | 4.88 |
| lung tumor | −7.46 |
| lung tumor | 2.18 |
| lung tumor | −12.41 |
| lung tumor | 2.97 |
| breast tumor | −5.76 |
| breast tumor | 16.16 |
| breast tumor | 1.53 |
| breast tumor | −1.09 |
| brain stage 5 ALZ | 1.11 |
| brain stage 5 ALZ | 1.32 |
| brain stage 5 ALZ | 1.16 |
| brain stage 5 ALZ | −4.83 |
| lung 24 COPD | −7.20 |
| lung 28 COPD | −4.86 |
| lung 23 COPD | −24.46 |
| asthmatic lung | −1.58 |
| asthmatic lung | 2.94 |
| asthmatic lung | 1.74 |
| asthmatic lung | 1.02 |
| endo VEGF | 7.35 |
| endo bFGF | 1.00 |
| heart (T-1) ischemic | −1.52 |
| heart (T-14) non-obstructive DCM | 1.14 |
| heart (T-3399) DCM | −1.46 |
| BM stim | 3.29 |
| osteo dif | 1.00 |
| OA Cartilage (pool) | −1.91 |
| PBL HIV IIIB | 2.38 |
| MRC5 HSV strain F | 8.43 |
| W12 cells | 1.00 |

Gene Name sbg1525809WNT8

Low overall expression. The highest normal expression is seen in the whole brain, testis, liver, parotid gland, and thymus. Some of the tissues representing the GI tract including the colon and the jejunum are represented as well as some of the tissue of the female reproductive system including the endometrium, ovary, and uterus. The highest disease expression is seen in the normal and disease brain samples. Upregulation in 1 of 4 breast tumor samples implicates this gene in breast cancer. Upregulation in the HSV-infected MRC5 cells suggests that this gene may be a host factor in HSV.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1525809WNT8 | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 29.74, 29.32 | 643.92 | 815.05 | 729.49 | 7.24 | 6.91 | 5037.88 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 40, 37.05 | 0 | 10.82 | 5.41 | 2.71 | 18.45 | 99.82 |
| Endometrium | 37.21, 40 | 9.89 | 0 | 4.95 | 0.73 | 68.21 | 337.31 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 33.11, 36.95 | 98.1 | 11.44 | 54.77 | 6.60 | 7.58 | 414.92 |
| Kidney | 40, 40 | 0 | 0 | 0.00 | 2.12 | 23.58 | 0.00 |
| Liver | 35.45, 40 | 26.55 | 0 | 13.28 | 1.50 | 33.33 | 442.50 |
| Fetal Liver Clontech | 40, 40 | 0 | 0 | 0.00 | 10.40 | 4.81 | 0.00 |
| Lung | 40, 40 | 0 | 0 | 0.00 | 2.57 | 19.46 | 0.00 |
| Mammary Gland | 40, 40 | 0 | 0 | 0.00 | 13.00 | 3.85 | 0.00 |
| Clontech | | | | | | | |
| Myometrium | 40, 40 | 0 | 0 | 0.00 | 2.34 | 21.37 | 0.00 |
| Omentum | 40, 40 | 0 | 0 | 0.00 | 3.94 | 12.69 | 0.00 |
| Ovary | 35.51, 36.3 | 25.61 | 16.49 | 21.05 | 4.34 | 11.52 | 242.51 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 36.18, 36.67 | 17.64 | 13.4 | 15.52 | 5.48 | 9.12 | 141.61 |
| Placenta Clontech | 40, 40 | 0 | 0 | 0.00 | 5.26 | 9.51 | 0.00 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland Clontech | 40, 40 | 0 | 0 | 0.00 | 7.31 | 6.84 | 0.00 |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 40, 40 | 0 | 0 | 0.00 | 4.92 | 10.16 | 0.00 |
| Stomach | 40, 40 | 0 | 0 | 0.00 | 2.73 | 18.32 | 0.00 |
| Testis Clontech | 40, 37.35 | 0 | 9.13 | 4.57 | 0.57 | 87.87 | 401.14 |
| Thymus Clontech | 37.64, 35.26 | 7.78 | 29.5 | 18.64 | 9.89 | 5.06 | 94.24 |
| Thyroid | 40, 40 | 0 | 0 | 0.00 | 2.77 | 18.05 | 0.00 |
| Trachea Clontech | 37.13, 40 | 10.38 | 0 | 5.19 | 9.71 | 5.15 | 26.73 |
| Urinary Bladder | 40, 40 | 0 | 0 | 0.00 | 5.47 | 9.14 | 0.00 |
| Uterus | 36.99, 35.76 | 11.22 | 22.23 | 16.73 | 5.34 | 9.36 | 156.60 |
| genomic | 28.79 | 1094.71 | | | | | |
| b-actin | 27.79 | 1919.17 | | | | | |
| 1.00E+05 | 20.91 | 100000 | | | | | |
| 1.00E+05 | 21.03 | 100000 | | | | | |
| 1.00E+04 | 24.51 | 10000 | | | | | |
| 1.00E+04 | 24.59 | 10000 | | | | | |
| 1.00E+03 | 28.47 | 1000 | | | | | |
| 1.00E+03 | 28.76 | 1000 | | | | | |
| 1.00E+02 | 34.37 | 100 | | | | | |
| 1.00E+02 | 32.84 | 100 | | | | | |
| 1.00E+01 | 37.11 | 10 | | | | | |
| 1.00E+01 | 36.96 | 10 | | | | | |
| 1.00E+00 | 40 | 0 | | | | | |
| 1.00E+00 | 40 | 1 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1525809WNT8 | identifier) | Ct | copies | RNA | Sample | Population |
| colon normal GW98-167 | 21941 | 36.83 | 7.92 | 15.84 | colon normal | |
| colon tumor GW98-166 | 21940 | 37.72 | 5.11 | 10.23 | colon tumor | −1.55 |
| colon normal GW98-178 | 22080 | 40 | 1.75 | 3.49 | colon normal | |
| colon tumor GW98-177 | 22060 | 37.27 | 6.37 | 12.74 | colon tumor | 3.65 |
| colon normal GW98-561 | 23514 | 36.48 | 9.43 | 18.87 | colon normal | |
| colon tumor GW98-560 | 23513 | 39.67 | 2.03 | 4.06 | colon tumor | −4.64 |
| colon normal GW98-894 | 24691 | 36.16 | 11.09 | 22.17 | colon normal | |
| colon tumor GW98-893 | 24690 | 39.74 | 1.97 | 3.93 | colon tumor | −5.64 |
| lung normal GW98-3 | 20742 | 39.11 | 2.64 | 5.27 | lung normal | |
| lung tumor GW98-2 | 20741 | 40 | 1.75 | 3.49 | lung tumor | −1.51 |
| lung normal GW97-179 | 20677 | 37.48 | 5.75 | 11.50 | lung normal | |
| lung tumor GW97-178 | 20676 | 35.75 | 13.66 | 27.32 | lung tumor | 2.38 |
| lung normal GW98-165 | 21922 | 36.45 | 9.58 | 19.15 | lung normal | |
| lung tumor GW98-164 | 21921 | 35.43 | 16.11 | 32.21 | lung tumor | 1.68 |
| lung normal GW98-282 | 22584 | 37.39 | 6.01 | 12.01 | lung normal | |
| lung tumor GW98-281 | 22583 | 40 | 1.75 | 3.49 | lung tumor | −3.44 |
| breast normal GW00-392 | 28750 | 40 | 1.75 | 1.75 | breast normal | |
| breast tumor GW00-391 | 28746 | 33.26 | 51.26 | 102.52 | breast tumor | 58.73 |
| breast normal GW00-413 | 28798 | 37.05 | 7.10 | 7.10 | breast normal | |
| breast tumor GW00-412 | 28797 | 36.5 | 9.34 | 18.68 | breast tumor | 2.63 |
| breast normal GW00- | 27592-95 | 37.45 | 5.83 | 5.83 | breast normal | |
| 235:238 | | | | | | |
| breast tumor GW00- | 27588-91 | 35.98 | 12.15 | 12.15 | breast tumor | 2.08 |
| 231:234 | | | | | | |
| breast normal GW98-621 | 23656 | 35.2 | 18.15 | 36.29 | breast normal | |
| breast tumor GW98-620 | 23655 | 39.59 | 2.11 | 4.22 | breast tumor | −8.61 |
| brain normal BB99-542 | 25507 | 30.39 | 267.68 | 535.36 | brain normal | |
| brain normal BB99-406 | 25509 | 30.94 | 192.72 | 385.45 | brain normal | |
| brain normal BB99-904 | 25546 | 32.17 | 94.37 | 188.74 | brain normal | |
| brain stage 5 ALZ BB99- | 25502 | 30.61 | 234.55 | 469.10 | brain stage 5 ALZ | 1.27 |
| 874 | | | | | | |
| brain stage 5 ALZ BB99- | 25503 | 30.11 | 317.14 | 634.27 | brain stage 5 ALZ | 1.71 |
| 887 | | | | | | |
| brain stage 5 ALZ BB99- | 25504 | 30.57 | 240.24 | 480.47 | brain stage 5 ALZ | 1.30 |
| 862 | | | | | | |
| brain stage 5 ALZ BB99- | 25542 | 29.5 | 461.38 | 922.75 | brain stage 5 ALZ | 2.49 |
| 927 | | | | | | |
| CT lung | normal | 40 | 1.75 | 3.49 | CT lung Nml | |
| lung 26 | normal | 33.69 | 40.51 | | lung 26 Nml | |
| lung 27 | normal | 40 | 1.75 | 1.75 | lung 27 Nml | |
| lung 24 | COPD | 40 | 1.75 | 1.75 | lung 24 COPD | −1.33 |
| lung 28 | COPD | 40 | 1.75 | 1.75 | lung 28 COPD | −1.33 |
| lung 23 | COPD | 40 | 1.75 | 1.75 | lung 23 COPD | −1.33 |
| lung 25 | normal | 40 | 1.75 | 1.75 | lung 25 Nml | |
| asthmatic lung ODO3112 | 29321 | 38.56 | 3.42 | 3.42 | asthmatic lung | 1.47 |
| asthmatic lung ODO3433 | 29323 | 40 | 1.75 | 3.49 | asthmatic lung | 1.50 |
| asthmatic lung ODO3397 | 29322 | 40 | 1.75 | 3.49 | asthmatic lung | 1.50 |
| asthmatic lung ODO4928 | 29325 | 40 | 1.75 | 3.49 | asthmatic lung | 1.50 |
| endo cells | control | 40 | 1.75 | 1.75 | endo cells | |
| endo VEGF | | 40 | 1.75 | 1.75 | endo VEGF | 1.00 |
| endo bFGF | | 37.41 | 5.95 | 5.95 | endo bFGF | 3.41 |
| heart Clontech | normal | 40 | 1.75 | 3.49 | heart | |
| heart (T-1) ischemic | 29417 | 40 | 1.75 | 3.49 | heart (T-1) | 1.00 |
| | | | | | ischemic | |
| heart (T-14) non- | 29422 | 40 | 1.75 | 3.49 | heart (T-14) non- | 1.00 |
| obstructive DCM | | | | | obstructive DCM | |
| heart (T-3399) DCM | 29426 | 40 | 1.75 | 3.49 | heart (T-3399) | 1.00 |
| | | | | | DCM | |
| adenoid GW99-269 | 26162 | 40 | 1.75 | 3.49 | adenoid | |
| tonsil GW98-280 | 22582 | 35.4 | 16.36 | 32.72 | tonsil | |
| T cells PC00314 | 28453 | 40 | 1.75 | 3.49 | T cells | |
| PBMNC | | 40 | 1.75 | 1.75 | PBMNC | |
| monocyte | | 40 | 1.75 | 3.49 | monocyte | |
| B cells PC00665 | 28455 | 38.05 | 4.36 | 8.72 | B cells | |
| dendritic cells | 28441 | 40 | 1.75 | 3.49 | dendritic cells | |
| neutrophils | 28440 | 40 | 1.75 | 1.75 | neutrophils | |
| eosinophils | 28446 | 40 | 1.75 | 3.49 | eosinophils | |
| BM unstim | | 40 | 1.75 | 1.75 | BM unstim | |
| BM stim | treated | 40 | 1.75 | 1.75 | BM stim | 1.00 |
| osteo dif | treated | 40 | 1.75 | 1.75 | osteo dif | 1.00 |
| osteo undif | | 40 | 1.75 | 1.75 | osteo undif | |
| chondrocytes | | 40 | 1.75 | 4.36 | chondrocytes | |
| OA Synovium IP12/01 | 29462 | 35.9 | 12.65 | 12.65 | OA Synovium | |
| OA Synovium NP10/01 | 29461 | 40 | 1.75 | 3.49 | OA Synovium | |
| OA Synovium NP57/00 | 28464 | 34.81 | 22.26 | 44.51 | OA Synovium | |
| RA Synovium | 28466 | 40 | 1.75 | 3.49 | RA Synovium | |
| NP03/01 | | | | | | |
| RA Synovium | 28467 | 36.39 | 9.87 | 19.74 | RA Synovium | |
| NP71/00 | | | | | | |
| RA Synovium | 28475 | 40 | 1.75 | 3.49 | RA Synovium | |
| NP45/00 | | | | | | |
| OA bone (biobank) | 29217 | 40 | 1.75 | 1.75 | OA bone (biobank) | |
| OA bone Sample 1 | J. Emory | 40 | 1.75 | 3.49 | OA bone | |
| OA bone Sample 2 | J. Emory | 36.13 | 11.26 | 22.51 | OA bone | |
| Cartilage (pool) | Normal | 36.51 | 9.29 | 18.59 | Nml Cartilage | |
| | | | | | (pool) | |
| Cartilage (pool) | OA | 40 | 1.75 | 3.49 | OA Cartilage (pool) | −5.32 |
| PBL unifected | 28441 | 40 | 1.75 | 3.49 | PBL unifected | |
| PBL HIV IIIB | 28442 | 40 | 1.75 | 3.49 | PBL HIV IIIB | 1.00 |
| MRC5 uninfected | 29158 | 38.07 | 4.32 | 8.64 | MRC5 uninfected | |
| (100%) | | | | | (100%) | |
| MRC5 HSV strain F | 29178 | 32.86 | 63.97 | 127.95 | MRC5 HSV strain F | 14.82 |
| W12 cells | 29179 | 40 | 1.75 | 3.49 | W12 cells | 1.00 |
| Keratinocytes | 29180 | 40 | 1.75 | 3.49 | Keratinocytes | |
| B-actin control | | 27.57 | 1593.28 | | | |
| genomic | | 28.2 | 1053.33 | | | |
| 1.00E+05 | | 40 | 1.75 | | Disease plate did not have | |
| | | | | | genomic samples. | |
| 1.00E+05 | | 40 | 1.75 | | Copy number was, therefore | |
| | | | | | calculated from the gene- | |
| | | | | | specific genomic curve on the | |
| | | | | | normal plate. | |
| 1.00E+04 | | 40 | 1.75 | | | |
| 1.00E+04 | | 40 | 1.75 | | | |
| 1.00E+03 | | 40 | 1.75 | | | |
| 1.00E+03 | | 40 | 1.75 | | | |
| 1.00E+02 | | 40 | 1.75 | | | |
| 1.00E+02 | | 40 | 1.75 | | | |
| 1.00E+01 | | 40 | 1.75 | | | |
| 1.00E+01 | | 40 | 1.75 | | | |
| 1.00E+00 | | 40 | 1.75 | | | |
| 1.00E+00 | | 40 | 1.75 | | | |
| NTC | | 40 | 1.75 | | | |
| NTC | | 40 | 1.75 | | | |

*lung 26 Normal has been omitted due to multiple amplification failures from that sample

Gene Name sbg1525809WNT8

| | Fold Change in Disease |
|---|---|
| | Population Relative to |
| Disease tissues | Normal |
| colon tumor | −1.55 |
| colon tumor | 3.65 |
| colon tumor | −4.64 |
| colon tumor | −5.64 |
| lung tumor | −1.51 |
| lung tumor | 2.38 |
| lung tumor | 1.68 |
| lung tumor | −3.44 |
| breast tumor | 58.73 |
| breast tumor | 2.63 |
| breast tumor | 2.08 |
| breast tumor | −8.61 |
| brain stage 5 ALZ | 1.27 |
| brain stage 5 ALZ | 1.71 |
| brain stage 5 ALZ | 1.30 |
| brain stage 5 ALZ | 2.49 |
| lung 24 COPD | −1.33 |
| lung 28 COPD | −1.33 |
| lung 23 COPD | −1.33 |
| asthmatic lung | 1.47 |
| asthmatic lung | 1.50 |
| asthmatic lung | 1.50 |
| asthmatic lung | 1.50 |
| endo VEGF | 1.00 |
| endo bFGF | 3.41 |
| heart (T-1) ischemic | 1.00 |
| heart (T-14) non-obstructive DCM | 1.00 |
| heart (T-3399) DCM | 1.00 |
| BM stim | 1.00 |
| osteo dif | 1.00 |
| OA Cartilage (pool) | −5.32 |
| PBLHIV IIIB | 1.00 |
| MRC5 HSV strain F | 14.82 |
| W12 cells | 1.00 |

Gene Name sbg1519904cdk

Moderate to high overall expression. The highest normal expression is seen in the whole brain, fetal liver, and thymus. Additional high expression is seen in the subcutaneous adipocytes, the salivary gland and many of the tissues representing the female reproductive system including the mammary gland, the placenta, and the myometrium. This gene is expressed fairly ubiquitously in the disease samples with the highest expression seen in the T cells, uninfected PBLS, and the W12 cells. Upregulation in 2 of 4 breast tumor samples implicates this gene in breast cancer. Upregulation in 1 of 4 asthmatic lung samples indicates a possible involvement in asthma Upregulation in 3 of 3 disease heart samples implies an involvement in cardiovascular diseases such as non-obstructive and obstructive DCM and ischemia. Upregulated in the stimulated bone marrow and the differentiated osteoblasts. May be implicated in diseases of the bone such as osteoporosis. Downregulation in the HIV-infected PBLs and the HSV-infected MRC5 cells suggests that this gene may be a host factor in HIV and HSV.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1519904cdk | 1 and 2) | 1) | 2) | Copies | (ng) | (ng) | RNA |
| Subcutaneous | 31.33, 31.31 | 70.47 | 71.37 | 70.92 | 3.06 | 16.34 | 1158.82 |
| Adipocytes Zenbio | | | | | | | |
| Subcutaneous Adipose | 38.32, 35.71 | 1.13 | 5.28 | 3.21 | 0.96 | 52.36 | 167.80 |
| Zenbio | | | | | | | |
| Adrenal Gland Clontech | 38.53, 35.13 | 1 | 7.43 | 4.22 | 0.61 | 81.97 | 345.49 |
| Whole Brain Clontech | 23.3, 23.29 | 8125.17 | 8212.8 | 8168.99 | 7.24 | 6.91 | 56415.64 |
| Fetal Brain Clontech | 40, 37.83 | 0 | 1.51 | 0.76 | 0.48 | 103.95 | 78.48 |
| Cerebellum Clontech | 33.54, 32.7 | 19 | 31.38 | 25.19 | 2.17 | 23.04 | 580.41 |
| Cervix | 39.09, 34.53 | 0.71 | 10.59 | 5.65 | 2.42 | 20.66 | 116.74 |
| Colon | 32.65, 33.21 | 32.3 | 23.22 | 27.76 | 2.71 | 18.45 | 512.18 |
| Endometrium | 40, 35.31 | 0 | 6.67 | 3.34 | 0.73 | 68.21 | 227.49 |
| Esophagus | 33.86, 34.19 | 15.77 | 12.95 | 14.36 | 1.37 | 36.50 | 524.09 |
| Heart Clontech | 35.18, 40 | 7.23 | 0 | 3.62 | 1.32 | 37.88 | 136.93 |
| Hypothalamus | 40, 40 | 0 | 0 | 0.00 | 0.32 | 155.28 | 0.00 |
| Ileum | 40, 34.3 | 0 | 12.17 | 6.09 | 2.58 | 19.38 | 117.93 |
| Jejunum | 31.16, 32.16 | 77.78 | 42.98 | 60.38 | 6.60 | 7.58 | 457.42 |
| Kidney | 34.24, 35.35 | 12.57 | 6.54 | 9.56 | 2.12 | 23.58 | 225.35 |
| Liver | 35.96, 40 | 4.56 | 0 | 2.28 | 1.50 | 33.33 | 76.00 |
| Fetal Liver Clontech | 27.16, 27.02 | 828.81 | 901.45 | 865.13 | 10.40 | 4.81 | 4159.28 |
| Lung | 33.27, 32.96 | 22.35 | 26.8 | 24.58 | 2.57 | 19.46 | 478.11 |
| Mammary Gland | 29.23, 28.79 | 243.81 | 317.27 | 280.54 | 13.00 | 3.85 | 1079.00 |
| Clontech | | | | | | | |
| Myometrium | 33.91, 32.24 | 15.32 | 41.01 | 28.17 | 2.34 | 21.37 | 601.82 |
| Omentum | 40, 33.65 | 0 | 17.84 | 8.92 | 3.94 | 12.69 | 113.20 |
| Ovary | 33.95, 33.51 | 14.91 | 19.39 | 17.15 | 4.34 | 11.52 | 197.58 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 32.16, 31.84 | 43.01 | 52.19 | 47.60 | 5.48 | 9.12 | 434.31 |
| Placenta Clontech | 31.2, 31.32 | 76.24 | 70.84 | 73.54 | 5.26 | 9.51 | 699.05 |
| Prostate | 34.02, 33.02 | 14.34 | 25.92 | 20.13 | 3.00 | 16.67 | 335.50 |
| Rectum | 34.27, 37.67 | 12.38 | 1.66 | 7.02 | 1.23 | 40.65 | 285.37 |
| Salivary Gland Clontech | 30.75, 30.63 | 99.33 | 106.86 | 103.10 | 7.31 | 6.84 | 705.16 |
| Skeletal Muscle | 36.12, 36.3 | 4.13 | 3.73 | 3.93 | 1.26 | 39.68 | 155.95 |
| Clontech | | | | | | | |
| Skin | 35.5, 34.03 | 5.99 | 14.23 | 10.11 | 1.21 | 41.32 | 417.77 |
| Small Intestine Clontech | 35.23, 39.47 | 7.02 | 0.57 | 3.80 | 0.98 | 51.07 | 193.82 |
| Spleen | 34.43, 35.07 | 11.28 | 7.71 | 9.50 | 4.92 | 10.16 | 96.49 |
| Stomach | 35.12, 40 | 7.5 | 8.77 | 8.14 | 2.73 | 18.32 | 148.99 |
| Testis Clontech | 40, 39.08 | 0 | 0.72 | 0.36 | 0.57 | 87.87 | 31.63 |
| Thymus Clontech | 27.04, 26.44 | 893.2 | 1274.29 | 1083.75 | 9.89 | 5.06 | 5478.99 |
| Thyroid | 35.48, 33.05 | 6.03 | 25.39 | 15.71 | 2.77 | 18.05 | 283.57 |
| Trachea Clontech | 30.24, 30.39 | 134.17 | 122.65 | 128.41 | 9.71 | 5.15 | 661.23 |
| Urinary Bladder | 40, 32.22 | 0 | 41.7 | 20.85 | 5.47 | 9.14 | 190.59 |
| Uterus | 32.01, 33.06 | 47.08 | 25.38 | 36.23 | 5.34 | 9.36 | 339.23 |
| genomic | 26.3 | 1379.25 | | | | | |
| b-actin | 27.49 | 683.1 | | | | | |
| 1.00E+05 | 19.36 | 100000 | | | | | |
| 1.00E+05 | 19.42 | 100000 | | | | | |
| 1.00E+04 | 22.84 | 10000 | | | | | |
| 1.00E+04 | 22.8 | 10000 | | | | | |
| 1.00E+03 | 26.49 | 1000 | | | | | |
| 1.00E+03 | 26.45 | 1000 | | | | | |
| 1.00E+02 | 31.23 | 100 | | | | | |
| 1.00E+02 | 29.95 | 100 | | | | | |
| 1.00E+01 | 33.93 | 10 | | | | | |
| 1.00E+01 | 35.99 | 10 | | | | | |
| 1.00E+00 | 40 | 1 | | | | | |
| 1.00E+00 | 40 | 0 | | | | | |
| NTC | 40 | −1 | | | | | |
| NTC | 37.4 | −1 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1519904cdk | identifier) | Ct | copies | RNA | Sample | Population |
| colon normal GW98-167 | 21941 | 25.08 | 2125.15 | 4250.29 | colon normal | |
| colon tumor GW98-166 | 21940 | 25.01 | 2223.66 | 4447.32 | colon tumor | 1.05 |
| colon normal GW98-178 | 22080 | 27.63 | 442.18 | 884.35 | colon normal | |
| colon tumor GW98-177 | 22060 | 26.06 | 1141.56 | 2283.13 | colon tumor | 2.58 |
| colon normal GW98-561 | 23514 | 26.26 | 1008.48 | 2016.97 | colon normal | |
| colon tumor GW98-560 | 23513 | 26.24 | 1021.02 | 2042.04 | colon tumor | 1.01 |
| colon normal GW98-894 | 24691 | 25.22 | 1941.75 | 3883.51 | colon normal | |
| colon tumor GW98-893 | 24690 | 25.04 | 2180.86 | 4361.72 | colon tumor | 1.12 |
| lung normal GW98-3 | 20742 | 25.13 | 2057.62 | 4115.24 | lung normal | |
| lung tumor GW98-2 | 20741 | 27.03 | 631.22 | 1262.44 | lung tumor | −3.26 |
| lung normal GW97-179 | 20677 | 24.81 | 2532.80 | 5065.59 | lung normal | |
| lung tumor GW97-178 | 20676 | 25.16 | 2018.20 | 4036.41 | lung tumor | −1.25 |
| lung normal GW98-165 | 21922 | 25 | 2238.12 | 4476.25 | lung normal | |
| lung tumor GW98-164 | 21921 | 24.61 | 2887.95 | 5775.90 | lung tumor | 1.29 |
| lung normal GW98-282 | 22584 | 26.08 | 1127.45 | 2254.91 | lung normal | |
| lung tumor GW98-281 | 22583 | 26.67 | 784.49 | 1568.98 | lung tumor | −1.44 |
| breast normal GW00-392 | 28750 | 25.78 | 1360.07 | 1360.07 | breast normal | |
| breast tumor GW00-391 | 28746 | 25.58 | 1543.11 | 3086.23 | breast tumor | 2.27 |
| breast normal GW00-413 | 28798 | 28.12 | 332.52 | 332.52 | breast normal | |
| breast tumor GW00-412 | 28797 | 26.28 | 996.11 | 1992.23 | breast tumor | 5.99 |
| breast normal GW00- | 27592-95 | 28.65 | 245.67 | 245.67 | breast normal | |
| 235:238 | | | | | | |
| breast tumor GW00- | 27588-91 | 25.79 | 1351.55 | 1351.55 | breast tumor | 5.50 |
| 231:234 | | | | | | |
| breast normal GW98-621 | 23656 | 25.15 | 2031.25 | 4062.51 | breast normal | |
| breast tumor GW98-620 | 23655 | 24.59 | 2926.27 | 5852.54 | breast tumor | 1.44 |
| brain normal BB99-542 | 25507 | 24.69 | 2739.93 | 5479.85 | brain normal | |
| brain normal BB99-406 | 25509 | 26.27 | 1002.28 | 2004.56 | brain normal | |
| brain normal BB99-904 | 25546 | 26.21 | 1040.13 | 2080.26 | brain normal | |
| brain stage 5 ALZ BB99- | 25502 | 27.76 | 409.77 | 819.55 | brain stage 5 ALZ | −3.89 |
| 874 | | | | | | |
| brain stage 5 ALZ BB99- | 25503 | 24.73 | 2668.95 | 5337.90 | brain stage 5 ALZ | 1.67 |
| 887 | | | | | | |
| brain stage 5 ALZ BB99- | 25504 | 25.54 | 1582.77 | 3165.53 | brain stage 5 ALZ | −1.01 |
| 862 | | | | | | |
| brain stage 5 ALZ BB99- | 25542 | 25.72 | 1412.43 | 2824.86 | brain stage 5 ALZ | −1.13 |
| 927 | | | | | | |
| CT lung | normal | 26.28 | 996.11 | 1992.23 | CT lung Nml | |
| lung 26 | normal | 31.37 | 56.46 | | lung 26 Nml | |
| lung 27 | normal | 31.95 | 41.95 | 41.95 | lung 27 Nml | |
| lung 24 | COPD | 33.17 | 22.85 | 22.85 | lung 24 COPD | −30.21 |
| lung 28 | COPD | 31.09 | 65.29 | 65.29 | lung 28 COPD | −10.57 |
| lung 23 | COPD | 31.27 | 59.46 | 59.46 | lung 23 COPD | −11.61 |
| lung 25 | normal | 32.21 | 36.79 | 36.79 | lung 25 Nml | |
| asthmatic lung ODO3112 | 29321 | 27.83 | 393.38 | 393.38 | asthmatic lung | −1.75 |
| asthmatic lung ODO3433 | 29323 | 26.47 | 886.31 | 1772.62 | asthmatic lung | 2.57 |
| asthmatic lung ODO3397 | 29322 | 25.24 | 1916.96 | 3833.92 | asthmatic lung | 5.55 |
| asthmatic lung ODO4928 | 29325 | 26.5 | 870.18 | 1740.37 | asthmatic lung | 2.52 |
| endo cells | control | 29.11 | 189.76 | 189.76 | endo cells | |
| endo VEGF | | 29.15 | 185.58 | 185.58 | endo VEGF | −1.02 |
| endo bFGF | | 28.83 | 221.95 | 221.95 | endo bFGF | 1.17 |
| heart Clontech | normal | 27.41 | 503.37 | 1006.73 | heart | |
| heart (T-1) ischemic | 29417 | 25.9 | 1261.43 | 2522.86 | heart (T-1) | 2.51 |
| | | | | | ischemic | |
| heart (T-14) non- | 29422 | 25.73 | 1403.56 | 2807.12 | heart (T-14) non- | 2.79 |
| obstructive DCM | | | | | obstructive DCM | |
| heart (T-3399) DCM | 29426 | 25.98 | 1199.91 | 2399.82 | heart (T-3399) | 2.38 |
| | | | | | DCM | |
| adenoid GW99-269 | 26162 | 27.36 | 518.49 | 1036.98 | adenoid | |
| tonsil GW98-280 | 22582 | 25.81 | 1334.67 | 2669.33 | tonsil | |
| T cells PC00314 | 28453 | 24.02 | 4280.04 | 8560.08 | T cells | |
| PBMNC | | 33.82 | 16.68 | 16.68 | PBMNC | |
| monocyte | | 32.42 | 33.11 | 66.21 | monocyte | |
| B cells PC00665 | 28455 | 25.34 | 1797.92 | 3595.85 | B cells | |
| dendritic cells | 28441 | 26.54 | 849.16 | 1698.33 | dendritic cells | |
| neutrophils | 28440 | 24.97 | 2282.12 | 2282.12 | neutrophils | |
| eosinophils | 28446 | 26.2 | 1046.59 | 2093.17 | eosinophils | |
| BM unstim | | 30.6 | 84.47 | 84.47 | BM unstim | |
| BM stim | treated | 28.86 | 218.24 | 218.24 | BM stim | 2.58 |
| osteo dif | treated | 29.59 | 145.56 | 145.56 | osteo dif | 2.53 |
| osteo undif | | 31.33 | 57.64 | 57.64 | osteo undif | |
| chondrocytes | | 25.51 | 1613.22 | 4033.04 | chondrocytes | |
| OA Synovium IP12/01 | 29462 | 26.08 | 1127.45 | 1127.45 | OA Synovium | |
| OA Synovium NP10/01 | 29461 | 27.14 | 591.00 | 1181.99 | OA Synovium | |
| OA Synovium NP57/00 | 28464 | 25.83 | 1318.01 | 2636.02 | OA Synovium | |
| RA Synovium | 28466 | 26.05 | 1148.69 | 2297.38 | RA Synovium | |
| NP03/01 | | | | | | |
| RA Synovium | 28467 | 25.51 | 1613.22 | 3226.43 | RA Synovium | |
| NP71/00 | | | | | | |
| RA Synovium | 28475 | 25.74 | 1394.74 | 2789.49 | RA Synovium | |
| NP45/00 | | | | | | |
| OA bone (biobank) | 29217 | 27.53 | 468.95 | 468.95 | OA bone (biobank) | |
| OA bone Sample 1 | J. Emory | 26.81 | 720.65 | 1441.29 | OA bone | |
| OA bone Sample 2 | J. Emory | 26.87 | 695.00 | 1389.99 | OA bone | |
| Cartilage (pool) | Normal | 26.02 | 1170.35 | 2340.70 | Nml Cartilage | |
| | | | | | (pool) | |
| Cartilage (pool) | OA | 27.15 | 587.48 | 1174.95 | OA Cartilage | −1.99 |
| | | | | | (pool) | |
| PBL unifected | 28441 | 23.67 | 5429.99 | 10859.98 | PBL unifected | |
| PBL HIV IIIB | 28442 | 25.5 | 1623.50 | 3247.01 | PBL HIV IIIB | −3.34 |
| MRC5 uninfected | 29158 | 25.69 | 1439.41 | 2878.82 | MRC5 uninfected | |
| (100%) | | | | | (100%) | |
| MRC5 HSV strain F | 29178 | 29.72 | 135.57 | 271.15 | MRC5 HSV strain F | −10.62 |
| W12 cells | 29179 | 24.11 | 4028.24 | 8056.48 | W12 cells | 1.88 |
| Keratinocytes | 29180 | 25.07 | 2138.93 | 4277.86 | Keratinocytes | |
| B-actin control | | 27.39 | 509.36 | | | |
| genomic | | 26.99 | 646.56 | | | |
| 1.00E+05 | | 38.65 | 1.92 | Disease plate did not have genomic | | |
| | | | | samples. | | |
| 1.00E+05 | | 38.29 | 2.23 | Copy number was, therefore calculated | | |
| | | | | from the gene-specific genomic curve | | |
| | | | | on the normal plate. | | |
| 1.00E+04 | | 36.45 | 4.95 | | | |
| 1.00E+04 | | 38.78 | 1.81 | | | |
| 1.00E+03 | | 38.22 | 2.30 | | | |
| 1.00E+03 | | 39.34 | 1.44 | | | |
| 1.00E+02 | | 39.78 | 1.20 | | | |
| 1.00E+02 | | 39.65 | 1.27 | | | |
| 1.00E+01 | | 36.39 | 5.09 | | | |
| 1.00E+01 | | 39.94 | 1.12 | | | |
| 1.00E+00 | | 39.12 | 1.57 | | | |
| 1.00E+00 | | 38.04 | 2.48 | | | |
| NTC | | 40 | 1.10 | | | |

*lung 26 Normal has been omitted due to multiple amplification failures from that sample

Gene Name sbg1519904cdk

| | Fold Change in Disease |
|---|---|
| | Population Relative to |
| Disease tissues | Normal |
| colon tumor | 1.05 |
| colon tumor | 2.58 |
| colon tumor | 1.01 |
| colon tumor | 1.12 |
| lung tumor | −3.26 |
| lung tumor | −1.25 |
| lung tumor | 1.29 |
| lung tumor | −1.44 |
| breast tumor | 2.27 |
| breast tumor | 5.99 |
| breast tumor | 5.50 |
| breast tumor | 1.44 |
| brain stage 5 ALZ | −3.89 |
| brain stage 5 ALZ | 1.67 |
| brain stage 5 ALZ | −1.01 |
| brain stage 5 ALZ | −1.13 |
| lung 24 COPD | −30.21 |
| lung 28 COPD | −10.57 |
| lung 23 COPD | −11.61 |
| asthmatic lung | −1.75 |
| asthmatic lung | 2.57 |
| asthmatic lung | 5.55 |
| asthmatic lung | 2.52 |
| endo VEGF | −1.02 |
| endo bFGF | 1.17 |
| heart (T-1) ischemic | 2.51 |
| heart (T-14) non-obstructive DCM | 2.79 |
| heart (T-3399) DCM | 2.38 |
| BM stim | 2.58 |
| osteo dif | 2.53 |
| OA Cartilage (pool) | −1.99 |
| PBL HIV IIIB | −3.34 |
| MRC5 HSV strain F | −10.62 |
| W12 cells | 1.88 |

Gene Name sbg1521449connexin

Moderate to low overall expression. This gene is expressed fairly ubiquitously in the normal samples with highest levels of expression seen in the whole brain, hypothalamus, liver, fetal liver, and thymus. The highest disease expression is seen in the breast, colon, and lung normal and tumor samples and in the normal and disease brain samples. Upregulation in 2 of 4 breast tumor samples implicates this gene in breast cancer. Downregulation in 2 of 4 asthmatic lung samples indicates a possible involvement in asthma Upregulation in 3 of 3 disease heart samples implies an involvement in cardiovascular diseases such as non-obstructive and obstructive DCM and ischemia. Moderate to high expression in the RA and OA synovium samples and the chondrocytes with corroborating high expression in the T cells, B cells, dendritic cells, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1521449- | (sample | (sample | (sample | GOI | rRNA | rRNA | total |
| connexin | 1 and 2) | 1) | 2) | Copies | (ng) | (ng) | RNA |
| Subcutaneous | 38.05, 37.25 | 0.37 | 0.62 | 0.50 | 3.06 | 16.34 | 8.09 |
| Adipocytes Zenbio | | | | | | | |
| Subcutaneous Adipose | 39.32, 39.13 | 0.16 | 0.18 | 0.17 | 0.96 | 52.36 | 8.90 |
| Zenbio | | | | | | | |
| Adrenal Gland Clontech | 37.27, 36.02 | 0.61 | 1.4 | 1.01 | 0.61 | 81.97 | 82.38 |
| Whole Brain Clontech | 26.93, 26.74 | 579.86 | 654.12 | 616.99 | 7.24 | 6.91 | 4260.98 |
| Fetal Brain Clontech | 38.44, 36.08 | 0.28 | 1.35 | 0.82 | 0.48 | 103.95 | 84.72 |
| Cerebellum Clontech | 34.17, 33.86 | 4.77 | 5.89 | 5.33 | 2.17 | 23.04 | 122.81 |
| Cervix | 35.78, 34.45 | 1.64 | 3.97 | 2.81 | 2.42 | 20.66 | 57.95 |
| Colon | 37.58, 34.4 | 0.5 | 4.1 | 2.30 | 2.71 | 18.45 | 42.44 |
| Endometrium | 35.52, 36.32 | 1.95 | 1.15 | 1.55 | 0.73 | 68.21 | 105.73 |
| Esophagus | 37.2, 36.68 | 0.64 | 0.91 | 0.78 | 1.37 | 36.50 | 28.28 |
| Heart Clontech | 35.58, 35.61 | 1.88 | 1.84 | 1.86 | 1.32 | 37.88 | 70.45 |
| Hypothalamus | 35.78, 36.24 | 1.64 | 1.22 | 1.43 | 0.32 | 155.28 | 222.05 |
| Ileum | 36.89, 35.46 | 0.79 | 2.03 | 1.41 | 2.58 | 19.38 | 27.33 |
| Jejunum | 32.55, 32.37 | 13.94 | 15.76 | 14.85 | 6.60 | 7.58 | 112.50 |
| Kidney | 36.69, 36.32 | 0.9 | 1.15 | 1.03 | 2.12 | 23.58 | 24.17 |
| Liver | 33.4, 34.37 | 7.97 | 4.18 | 6.08 | 1.50 | 33.33 | 202.50 |
| Fetal Liver Clontech | 31.75, 31.56 | 23.79 | 26.88 | 25.34 | 10.40 | 4.81 | 121.80 |
| Lung | 35.02, 35.59 | 2.72 | 1.86 | 2.29 | 2.57 | 19.46 | 44.55 |
| Mammary Gland | 32.27, 32.71 | 16.81 | 12.59 | 14.70 | 13.00 | 3.85 | 56.54 |
| Clontech | | | | | | | |
| Myometrium | 35.06, 35.53 | 2.65 | 1.94 | 2.30 | 2.34 | 21.37 | 49.04 |
| Omentum | 32.33, 34.21 | 16.17 | 4.66 | 10.42 | 3.94 | 12.69 | 132.17 |
| Ovary | 33.74, 33.59 | 6.35 | 7 | 6.68 | 4.34 | 11.52 | 76.90 |
| Pancreas | 36.87, 36.58 | 0.8 | 0.97 | 0.89 | 0.81 | 61.80 | 54.70 |
| Head of Pancreas | 34.28, 36.68 | 4.46 | 0.91 | 2.69 | 1.57 | 31.85 | 85.51 |
| Parotid Gland | 34.09, 33.54 | 5.03 | 7.25 | 6.14 | 5.48 | 9.12 | 56.02 |
| Placenta Clontech | 35.07, 36.04 | 2.63 | 1.39 | 2.01 | 5.26 | 9.51 | 19.11 |
| Prostate | 35.42, 35.23 | 2.1 | 2.37 | 2.24 | 3.00 | 16.67 | 37.25 |
| Rectum | 36.31, 35.93 | 1.16 | 1.49 | 1.33 | 1.23 | 40.65 | 53.86 |
| Salivary Gland Clontech | 34.01, 33.49 | 5.3 | 7.48 | 6.39 | 7.31 | 6.84 | 43.71 |
| Skeletal Muscle | 37.12, 38.72 | 0.68 | 0.23 | 0.46 | 1.26 | 39.68 | 18.06 |
| Clontech | | | | | | | |
| Skin | 39.2, 37.09 | 0.17 | 0.69 | 0.43 | 1.21 | 41.32 | 17.77 |
| Small Intestine Clontech | 38.84, 37.18 | 0.22 | 0.65 | 0.44 | 0.98 | 51.07 | 22.22 |
| Spleen | 34.52, 34.67 | 3.79 | 3.43 | 3.61 | 4.92 | 10.16 | 36.69 |
| Stomach | 36.3, 35.98 | 1.16 | 1.44 | 1.30 | 2.73 | 18.32 | 23.81 |
| Testis Clontech | 37.29, 36.56 | 0.61 | 0.98 | 0.80 | 0.57 | 87.87 | 69.86 |
| Thymus Clontech | 31.3, 31.11 | 31.97 | 36.26 | 34.12 | 9.89 | 5.06 | 172.47 |
| Thyroid | 35.22, 36.32 | 2.39 | 1.15 | 1.77 | 2.77 | 18.05 | 31.95 |
| Trachea Clontech | 34.39, 34.02 | 4.13 | 5.27 | 4.70 | 9.71 | 5.15 | 24.20 |
| Urinary Bladder | 34.87, 34.25 | 3.01 | 4.53 | 3.77 | 5.47 | 9.14 | 34.46 |
| Uterus | 33.4, 32.98 | 7.98 | 10.51 | 9.25 | 5.34 | 9.36 | 86.56 |
| genomic | 25.67 | 1329.61 | | | | | |
| b-actin | 27.33 | 444.52 | | | | | |
| 1.00E+05 | 19.28 | 100000 | | | | | |
| 1.00E+05 | 19.4 | 100000 | | | | | |
| 1.00E+04 | 22.27 | 10000 | | | | | |
| 1.00E+04 | 22.19 | 10000 | | | | | |
| 1.00E+03 | 26.01 | 1000 | | | | | |
| 1.00E+03 | 25.8 | 1000 | | | | | |
| 1.00E+02 | 30.08 | 100 | | | | | |
| 1.00E+02 | 29.91 | 100 | | | | | |
| 1.00E+01 | 33.07 | 10 | | | | | |
| 1.00E+01 | 33.84 | 10 | | | | | |
| 1.00E+00 | 37.02 | 1 | | | | | |
| 1.00E+00 | 35.22 | 1 | − | | | | |
| NTC | 38.14 | −1 | | | | | |
| NTC | 37.85 | −1 | | | | | |

| | | | | copies of | | |
|---|---|---|---|---|---|---|
| | | | | mRNA | | Fold |
| | Reg | | | detected/ | | Change |
| Sample | number | | Mean | 50 ng | | in |
| sbg1521449- | (GSK | | GOI | total | | Disease |
| connexin | identifier) | Ct | copies | RNA | Sample | Population |
| colon normal GW98-167 | 21941 | 29.61 | 127.84 | 255.68 | colon normal | |
| colon tumor GW98-166 | 21940 | 29.7 | 120.56 | 241.12 | colon tumor | −1.06 |
| colon normal GW98-178 | 22080 | 31.16 | 47.88 | 95.76 | colon normal | |
| colon tumor GW98-177 | 22060 | 30.04 | 97.35 | 194.70 | colon tumor | 2.03 |
| colon normal GW98-561 | 23514 | 30.47 | 74.05 | 148.10 | colon normal | |
| colon tumor GW98-560 | 23513 | 30.59 | 68.46 | 136.92 | colon tumor | −1.08 |
| colon normal GW98-894 | 24691 | 29.36 | 148.99 | 297.98 | colon normal | |
| colon tumor GW98-893 | 24690 | 29.27 | 158.62 | 317.24 | colon tumor | 1.06 |
| lung normal GW98-3 | 20742 | 29.04 | 182.72 | 365.44 | lung normal | |
| lung tumor GW98-2 | 20741 | 31.3 | 43.67 | 87.34 | lung tumor | −4.18 |
| lung normal GW97-179 | 20677 | 30.21 | 87.43 | 174.86 | lung normal | |
| lung tumor GW97-178 | 20676 | 29.38 | 147.55 | 295.10 | lung tumor | 1.69 |
| lung normal GW98-165 | 21922 | 29.52 | 135.41 | 270.82 | lung normal | |
| lung tumor GW98-164 | 21921 | 30.07 | 95.25 | 190.50 | lung tumor | −1.42 |
| lung normal GW98-282 | 22584 | 31.11 | 49.34 | 98.68 | lung normal | |
| lung tumor GW98-281 | 22583 | 30.77 | 61.11 | 122.22 | lung tumor | 1.24 |
| breast normal GW00-392 | 28750 | 29.34 | 151.22 | 151.22 | breast normal | |
| breast tumor GW00-391 | 28746 | 28.76 | 217.94 | 435.88 | breast tumor | 2.88 |
| breast normal GW00-413 | 28798 | 30.46 | 74.23 | 74.23 | breast normal | |
| breast tumor GW00-412 | 28797 | 29.03 | 184.33 | 368.66 | breast tumor | 4.97 |
| breast normal GW00- | 27592-95 | 33.21 | 13 | 13.00 | breast normal | |
| 235:238 | | | | | | |
| breast tumor GW00- | 27588-91 | 30.17 | 89.28 | 89.28 | breast tumor | 6.87 |
| 231:234 | | | | | | |
| breast normal GW98-621 | 23656 | 29.48 | 138.9 | 277.80 | breast normal | |
| breast tumor GW98-620 | 23655 | 29.99 | 100.55 | 201.10 | breast tumor | −1.38 |
| brain normal BB99-542 | 25507 | 30.6 | 68.32 | 136.64 | brain normal | |
| brain normal BB99-406 | 25509 | 30.74 | 62.32 | 124.64 | brain normal | |
| brain normal BB99-904 | 25546 | 31.09 | 50.01 | 100.02 | brain normal | |
| brain stage 5 ALZ BB99- | 25502 | 31.03 | 51.88 | 103.76 | brain stage 5 ALZ | −1.16 |
| 874 | | | | | | |
| brain stage 5 ALZ BB99- | 25503 | 29.5 | 137 | 274.00 | brain stage 5 ALZ | 2.28 |
| 887 | | | | | | |
| brain stage 5 ALZ BB99- | 25504 | 30.04 | 97.32 | 194.64 | brain stage 5 ALZ | 1.62 |
| 862 | | | | | | |
| brain stage 5 ALZ BB99- | 25542 | 30.12 | 92.58 | 185.16 | brain stage 5 ALZ | 1.54 |
| 927 | | | | | | |
| CT lung | normal | 31.49 | 38.73 | 77.46 | CT lung Nml | |
| lung 26 | normal | 34.07 | 7.55 | | lung 26 Nml | |
| lung 27 | normal | 38.07 | 0.6 | 0.60 | lung 27 Nml | |
| lung 24 | COPD | 37.74 | 0.74 | 0.74 | lung 24 COPD | −36.11 |
| lung 28 | COPD | 36.85 | 1.3 | 1.30 | lung 28 COPD | −20.55 |
| lung 23 | COPD | 36.02 | 2.19 | 2.19 | lung 23 COPD | −12.20 |
| lung 25 | normal | 36.09 | 2.1 | 2.10 | lung 25 Nml | |
| asthmatic lung ODO3112 | 29321 | 35.54 | 2.98 | 2.98 | asthmatic lung | −8.97 |
| asthmatic lung ODO3433 | 29323 | 34.42 | 6.05 | 12.10 | asthmatic lung | −2.21 |
| asthmatic lung ODO3397 | 29322 | 31.9 | 29.9 | 59.80 | asthmatic lung | 2.24 |
| asthmatic lung ODO4928 | 29325 | 33.84 | 8.75 | 17.50 | asthmatic lung | −1.53 |
| endo cells | control | 34.17 | 7.08 | 7.08 | endo cells | |
| endo VEGF | | 34.73 | 4.96 | 4.96 | endo VEGF | −1.43 |
| endo bFGF | | 34.51 | 5.71 | 5.71 | endo bFGF | −1.24 |
| heart Clontech | normal | 35.07 | 4.01 | 8.02 | heart | |
| heart (T-1) ischemic | 29417 | 32.02 | 27.63 | 55.26 | heart (T-1) | 6.89 |
| | | | | | ischemic | |
| heart (T-14) non- | 29422 | 32.12 | 26.01 | 52.02 | heart (T-14) non- | 6.49 |
| obstructive DCM | | | | | obstructive DCM | |
| heart (T-3399) DCM | 29426 | 31.81 | 31.6 | 63.20 | heart (T-3399) | 7.88 |
| | | | | | DCM | |
| adenoid GW99-269 | 26162 | 32.06 | 27.08 | 54.16 | adenoid | |
| tonsil GW98-280 | 22582 | 31.65 | 35.03 | 70.06 | tonsil | |
| T cells PC00314 | 28453 | 29.97 | 101.75 | 203.50 | T cells | |
| PBMNC | | 35.68 | 2.72 | 2.72 | PBMNC | |
| monocyte | | 33.68 | 9.64 | 19.28 | monocyte | |
| B cells PC00665 | 28455 | 29.93 | 104.12 | 208.24 | B cells | |
| dendritic cells | 28441 | 30.83 | 58.85 | 117.70 | dendritic cells | |
| neutrophils | 28440 | 31.1 | 49.65 | 49.65 | neutrophils | |
| eosinophils | 28446 | 31.01 | 52.56 | 105.12 | eosinophils | |
| BM unstim | | 35.46 | 3.13 | 3.13 | BM unstim | |
| BM stim | treated | 34.87 | 4.55 | 4.55 | BM stim | 1.45 |
| osteo dif | treated | 36.01 | 2.2 | 2.20 | osteo dif | 1.95 |
| osteo undif | | 37.06 | 1.13 | 1.13 | osteo undif | |
| chondrocytes | | 31.4 | 40.95 | 102.38 | chondrocytes | |
| OA Synovium IP12/01 | 29462 | 34.18 | 7.04 | 7.04 | OA Synovium | |
| OA Synovium NP10/01 | 29461 | 32.08 | 26.64 | 53.28 | OA Synovium | |
| OA Synovium NP57/00 | 28464 | 31.17 | 47.32 | 94.64 | OA Synovium | |
| RA Synovium | 28466 | 31.12 | 49.1 | 98.20 | RA Synovium | |
| NP03/01 | | | | | | |
| RA Synovium | 28467 | 30.48 | 73.65 | 147.30 | RA Synovium | |
| NP71/00 | | | | | | |
| RA Synovium | 28475 | 31.52 | 37.97 | 75.94 | RA Synovium | |
| NP45/00 | | | | | | |
| OA bone (biobank) | 29217 | 33.84 | 8.73 | 8.73 | OA bone (biobank) | |
| OA bone Sample 1 | J. Emory | 33.02 | 14.69 | 29.38 | OA bone | |
| OA bone Sample 2 | J. Emory | 34 | 7.91 | 15.82 | OA bone | |
| Cartilage (pool) | Normal | 32.41 | 21.57 | 43.14 | Nml Cartilage | |
| | | | | | (pool) | |
| Cartilage (pool) | OA | 33.21 | 13.01 | 26.02 | OA Cartilage (pool) | −1.66 |
| PBL unifected | 28441 | 31.16 | 47.76 | 95.52 | PBL unifected | |
| PBL HIV IIIB | 28442 | 31.06 | 50.89 | 101.78 | PBL HIV IIIB | 1.07 |
| MRC5 uninfected | 29158 | 31.11 | 49.19 | 98.38 | MRC5 uninfected | |
| (100%) | | | | | (100%) | |
| MRC5 HSV strain F | 29178 | 32.04 | 27.34 | 54.68 | MRC5 HSV strain F | −1.80 |
| W12 cells | 29179 | 31.48 | 39.06 | 78.12 | W12 cells | 1.37 |
| Keratinocytes | 29180 | 31.98 | 28.42 | 56.84 | Keratinocytes | |
| B-actin control | | 27.14 | 609.73 | | | |
| genomic | | 25.24 | 2031.4 | | | |
| 1.00E+05 | | 19.93 | 100000 | | | |
| 1.00E+05 | | 19.87 | 100000 | | | |
| 1.00E+04 | | 22.49 | 10000 | | | |
| 1.00E+04 | | 22.37 | 10000 | | | |
| 1.00E+03 | | 24.73 | 1000 | | | |
| 1.00E+03 | | 24.83 | 1000 | | | |
| 1.00E+02 | | 30.26 | 100 | | | |
| 1.00E+02 | | 30.17 | 100 | | | |
| 1.00E+01 | | 35.69 | 10 | | | |
| 1.00E+01 | | 34.47 | 10 | | | |
| 1.00E+00 | | 37.49 | 1 | | | |
| 1.00E+00 | | 35.83 | 1 | | | |
| NTC | | 37.35 | −1 | | | |

*lung 26 Normal has been omitted due to multiple amplification failures from that sample

Gene Name sbg1521449connexin

| | Fold Change in Disease |
|---|---|
| | Population Relative to |
| Disease tissues | Normal |
| colon tumor | −1.06 |
| colon tumor | 2.03 |
| colon tumor | −1.08 |
| colon tumor | 1.06 |
| lung tumor | −4.18 |
| lung tumor | 1.69 |
| lung tumor | −1.42 |
| lung tumor | 1.24 |
| breast tumor | 2.88 |
| breast tumor | 4.97 |
| breast tumor | 6.87 |
| breast tumor | −1.38 |
| brain stage 5 ALZ | −1.16 |
| brain stage 5 ALZ | 2.28 |
| brain stage 5 ALZ | 1.62 |
| brain stage 5 ALZ | 1.54 |
| lung 24 COPD | −36.11 |
| lung 28 COPD | −20.55 |
| lung 23 COPD | −12.20 |
| asthmatic lung | −8.97 |
| asthmatic lung | −2.21 |
| asthmatic lung | 2.24 |
| asthmatic lung | −1.53 |
| endo VEGF | −1.43 |
| endo bFGF | −1.24 |
| heart (T-1) ischemic | 6.89 |
| heart (T-14) non-obstructive DCM | 6.49 |
| heart (T-3399) DCM | 7.88 |
| BM stim | 1.45 |
| osteo dif | 1.95 |
| OA Cartilage (pool) | −1.66 |
| PBL HIV IIIB | 1.07 |
| MRC5 HSV strain F | −1.80 |
| W12 cells | 1.37 |

Gene Name sbg1522659Siglec (Taqman was Performed for this Sample)

Moderate to high overall expression. The highest normal expression is seen in the myometrium, ovary, adrenal gland, whole brain, spleen, and fetal liver. The other tissues representing both the GI tract and the female reproductive system also show high expression. These tissues include the jejunum, omentum, small intestine, rectum, stomach, endometrium, and placenta. The GI pattern of expression suggests that this gene may be involved in diseases of the digestive tract such as IBS and Crohn's Disease. The highest disease expression is seen in the Clontech normal lung sample, some of the asthmatic lung samples, the Clontech normal heart sample, and all of the OA samples. Upregulation in 2 of 4 lung tumor samples implicates this gene in lung cancer. Downregulation in 3 of 3 disease heart samples implies an involvement in cardiovascular diseases such as non-obstructive and obstructive DCM and ischemia. Upregulated in the stimulated bone marrow sample. Downregulation in the OA cartilage pool suggests that this gene may be involved in or a consequence of osteoarthritis. Upregulation in the HSV-infected MRC5 cells suggests that this gene may be a host factor in HSV. Upregulation in the W12 cells suggests that this gene may be a host factor in HPV.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1522659Siglec | 1 and 2) | 1) | 2) | Copies | (ng) | (ng) | RNA |
| Subcutaneous | 32.79, 32.97 | 0.26 | 0.22 | 0.24 | 3.06 | 16.34 | 3.92 |
| Adipocytes Zenbio | | | | | | | |
| Subcutaneous Adipose | 32.36, 32.09 | 0.41 | 0.54 | 0.48 | 0.96 | 52.36 | 24.87 |
| Zenbio | | | | | | | |
| Adrenal Gland | 30.43, 30.5 | 2.82 | 2.63 | 2.73 | 0.61 | 81.97 | 223.36 |
| Clontech | | | | | | | |
| Whole Brain Clontech | 26.61, 26.6 | 127.82 | 129.82 | 128.82 | 7.24 | 6.91 | 889.64 |
| Fetal Brain Clontech | 27.17, 30.55 | 72.98 | 2.49 | 37.74 | 0.48 | 103.95 | 3922.56 |
| Cerebellum Clontech | 31.1, 31.54 | 1.44 | 0.93 | 1.19 | 2.17 | 23.04 | 27.30 |
| Cervix | 31.19, 31.05 | 1.32 | 1.51 | 1.42 | 2.42 | 20.66 | 29.24 |
| Colon | 30.75, 30.9 | 2.04 | 1.75 | 1.90 | 2.71 | 18.45 | 34.96 |
| Endometrium | 30.47, 30.71 | 2.71 | 2.12 | 2.42 | 0.73 | 68.21 | 164.73 |
| Esophagus | 30.06, 30.12 | 4.07 | 3.82 | 3.95 | 1.37 | 36.50 | 143.98 |
| Heart Clontech | 30.11, 30.33 | 3.88 | 3.11 | 3.50 | 1.32 | 37.88 | 132.39 |
| Hypothalamus | 30.08, 30.31 | 3.99 | 3.16 | 3.58 | 0.32 | 155.28 | 555.12 |
| Ileum | 31.29, 31.04 | 1.19 | 1.53 | 1.36 | 2.58 | 19.38 | 26.36 |
| Jejunum | 31.07, 31.19 | 1.48 | 1.31 | 1.40 | 6.60 | 7.58 | 10.57 |
| Kidney | 30.45, 30.84 | 2.75 | 1.87 | 2.31 | 2.12 | 23.58 | 54.48 |
| Liver | 30.2, 30.13 | 3.53 | 3.79 | 3.66 | 1.50 | 33.33 | 122.00 |
| Fetal Liver Clontech | 29.09, 28.81 | 10.71 | 14.2 | 12.46 | 10.40 | 4.81 | 59.88 |
| Lung | 30.77, 31.04 | 2 | 1.53 | 1.77 | 2.57 | 19.46 | 34.34 |
| Mammary Gland | 31.23, 31.23 | 1.26 | 1.26 | 1.26 | 13.00 | 3.85 | 4.85 |
| Clontech | | | | | | | |
| Myometrium | 30.34, 30.99 | 3.09 | 1.61 | 2.35 | 2.34 | 21.37 | 50.21 |
| Omentum | 31.03, 31.02 | 1.54 | 1.55 | 1.55 | 3.94 | 12.69 | 19.61 |
| Ovary | 28.69, 28.77 | 16.06 | 14.84 | 15.45 | 4.34 | 11.52 | 178.00 |
| Pancreas | 30.57, 30.38 | 2.45 | 2.95 | 2.70 | 0.81 | 61.80 | 166.87 |
| Head of Pancreas | 30.22, 30.97 | 3.45 | 1.63 | 2.54 | 1.57 | 31.85 | 80.89 |
| Parotid Gland | 31.25, 31.06 | 1.24 | 1.5 | 1.37 | 5.48 | 9.12 | 12.50 |
| Placenta Clontech | 30.75, 30.63 | 2.05 | 2.31 | 2.18 | 5.26 | 9.51 | 20.72 |
| Prostate | 30.45, 30.59 | 2.75 | 2.39 | 2.57 | 3.00 | 16.67 | 42.83 |
| Rectum | 30.3, 30.68 | 3.21 | 2.2 | 2.71 | 1.23 | 40.65 | 109.96 |
| Salivary Gland | 30.6, 31.04 | 2.36 | 1.53 | 1.95 | 7.31 | 6.84 | 13.30 |
| Clontech | | | | | | | |
| Skeletal Muscle | 30.82, 31.03 | 1.9 | 1.55 | 1.73 | 1.26 | 39.68 | 68.45 |
| Clontech | | | | | | | |
| Skin | 30.65, 30.33 | 2.25 | 3.11 | 2.68 | 1.21 | 41.32 | 110.74 |
| Small Intestine | 30.61, 30.64 | 2.35 | 2.29 | 2.32 | 0.98 | 51.07 | 118.49 |
| Clontech | | | | | | | |
| Spleen | 30.6, 30.7 | 2.38 | 2.14 | 2.26 | 4.92 | 10.16 | 22.97 |
| Stomach | 30.77, 30.97 | 2.01 | 1.63 | 1.82 | 2.73 | 18.32 | 33.33 |
| Testis Clontech | 30.26, 30.18 | 3.32 | 3.61 | 3.47 | 0.57 | 87.87 | 304.48 |
| Thymus Clontech | 28.57, 28.75 | 17.98 | 15.05 | 16.52 | 9.89 | 5.06 | 83.49 |
| Thyroid | 31, 30.75 | 1.59 | 2.03 | 1.81 | 2.77 | 18.05 | 32.67 |
| Trachea Clontech | 31.06, 30.99 | 1.49 | 1.6 | 1.55 | 9.71 | 5.15 | 7.96 |
| Urinary Bladder | 30.92, 30.81 | 1.72 | 1.93 | 1.83 | 5.47 | 9.14 | 16.68 |
| Uterus | 30.4, 30.95 | 2.89 | 1.66 | 2.28 | 5.34 | 9.36 | 21.30 |
| genomic | 24.94 | 680.71 | | | | | |
| b-actin | 27.61 | 47.14 | | | | | |
| 1.00E+05 | 19.42 | 100000 | | | | | |
| 1.00E+05 | 19.39 | 100000 | | | | | |
| 1.00E+04 | 22.09 | 10000 | | | | | |
| 1.00E+04 | 21.97 | 10000 | | | | | |
| 1.00E+03 | 24.5 | 1000 | | | | | |
| 1.00E+03 | 24.43 | 1000 | | | | | |
| 1.00E+02 | 28.5 | 100 | | | | | |
| 1.00E+02 | 28.27 | 100 | | | | | |
| 1.00E+01 | 28.83 | 10 | | | | | |
| 1.00E+01 | 31.14 | 10 | | | | | |
| 1.00E+00 | 29.91 | 1 | | | | | |
| 1.00E+00 | 30.02 | 1 | | | | | |
| NTC | 29.79 | −1 | | | | | |
| NTC | 30.05 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1522659Siglec | identifier) | Ct | copies | RNA | Sample | Population |
| colon normal GW98-167 | 21941 | 28.59 | 33.24 | 66.48 | colon normal | |
| colon tumor GW98-166 | 21940 | 28.78 | 27.91 | 55.82 | colon tumor | −1.19 |
| colon normal GW98-178 | 22080 | 30.22 | 7.7 | 15.40 | colon normal | |
| colon tumor GW98-177 | 22060 | 29.4 | 16.01 | 32.02 | colon tumor | 2.08 |
| colon normal GW98-561 | 23514 | 30.01 | 9.27 | 18.54 | colon normal | |
| colon tumor GW98-560 | 23513 | 30.15 | 8.14 | 16.28 | colon tumor | −1.14 |
| colon normal GW98-894 | 24691 | 28.96 | 23.84 | 47.68 | colon normal | |
| colon tumor GW98-893 | 24690 | 28.44 | 38.1 | 76.20 | colon tumor | 1.60 |
| lung normal GW98-3 | 20742 | 29.06 | 21.79 | 43.58 | lung normal | |
| lung tumor GW98-2 | 20741 | 30 | 9.37 | 18.74 | lung tumor | −2.33 |
| lung normal GW97-179 | 20677 | 29.96 | 9.73 | 19.46 | lung normal | |
| lung tumor GW97-178 | 20676 | 27.91 | 61.4 | 122.80 | lung tumor | 6.31 |
| lung normal GW98-165 | 21922 | 28.37 | 40.67 | 81.34 | lung normal | |
| lung tumor GW98-164 | 21921 | 29.21 | 19.05 | 38.10 | lung tumor | −2.13 |
| lung normal GW98-282 | 22584 | 30.06 | 8.88 | 17.76 | lung normal | |
| lung tumor GW98-281 | 22583 | 29.11 | 20.89 | 41.78 | lung tumor | 2.35 |
| breast normal GW00-392 | 28750 | 29.88 | 10.38 | 10.38 | breast normal | |
| breast tumor GW00-391 | 28746 | 29.83 | 10.89 | 21.78 | breast tumor | 2.10 |
| breast normal GW00-413 | 28798 | 31.21 | 3.16 | 3.16 | breast normal | |
| breast tumor GW00-412 | 28797 | 29.86 | 10.63 | 21.26 | breast tumor | 6.73 |
| breast normal GW00- | 27592-95 | 32.1 | 1.42 | 1.42 | breast normal | |
| 235:238 | | | | | | |
| breast tumor GW00- | 27588-91 | 31.73 | 1.97 | 1.97 | breast tumor | 1.39 |
| 231:234 | | | | | | |
| breast normal GW98-621 | 23656 | 29.11 | 20.78 | 41.56 | breast normal | |
| breast tumor GW98-620 | 23655 | 29.14 | 20.29 | 40.58 | breast tumor | −1.02 |
| brain normal BB99-542 | 25507 | 29.79 | 11.34 | 22.68 | brain normal | |
| brain normal BB99-406 | 25509 | 29.73 | 11.93 | 23.86 | brain normal | |
| brain normal BB99-904 | 25546 | 30.12 | 8.4 | 16.80 | brain normal | |
| brain stage 5 ALZ BB99- | 25502 | 29.88 | 10.44 | 20.88 | brain stage 5 ALZ | −1.01 |
| 874 | | | | | | |
| brain stage 5 ALZ BB99- | 25503 | 29.07 | 21.63 | 43.26 | brain stage 5 ALZ | 2.05 |
| 887 | | | | | | |
| brain stage 5 ALZ BB99- | 25504 | 29.29 | 17.74 | 35.48 | brain stage 5 ALZ | 1.68 |
| 862 | | | | | | |
| brain stage 5 ALZ BB99- | 25542 | 29.84 | 10.8 | 21.60 | brain stage 5 ALZ | 1.02 |
| 927 | | | | | | |
| CT lung | normal | 28.36 | 40.79 | 81.58 | CT lung Nml | |
| lung 26 | normal | 28.68 | 30.74 | | lung 26 Nml | |
| lung 27 | normal | 31.5 | 2.43 | 2.43 | lung 27 Nml | |
| lung 24 | COPD | 31.84 | 1.78 | 1.78 | lung 24 COPD | −16.29 |
| lung 28 | COPD | 31.63 | 2.16 | 2.16 | lung 28 COPD | −13.42 |
| lung 23 | COPD | 32.08 | 1.43 | 1.43 | lung 23 COPD | −20.27 |
| lung 25 | normal | 31.28 | 2.96 | 2.96 | lung 25 Nml | |
| asthmatic lung ODO3112 | 29321 | 31.03 | 3.7 | 3.70 | asthmatic lung | −7.84 |
| asthmatic lung ODO3433 | 29323 | 30.1 | 8.53 | 17.06 | asthmatic lung | −1.70 |
| asthmatic lung ODO3397 | 29322 | 28.3 | 43.06 | 86.12 | asthmatic lung | 2.97 |
| asthmatic lung ODO4928 | 29325 | 29.71 | 12.09 | 24.18 | asthmatic lung | −1.20 |
| endo cells | control | 31.53 | 2.37 | 2.37 | endo cells | |
| endo VEGF | | 32.48 | 1.01 | 1.01 | endo VEGF | −2.35 |
| endo bFGF | | 31.64 | 2.13 | 2.13 | endo bFGF | −1.11 |
| heart Clontech | normal | 28.78 | 28.16 | 56.32 | heart | |
| heart (T-1) ischemic | 29417 | 30.04 | 9.03 | 18.06 | heart (T-1) ischemic | −3.12 |
| heart (T-14) non- | 29422 | 30.52 | 5.85 | 11.70 | heart (T-14) non- | −4.81 |
| obstructive DCM | | | | | obstructive DCM | |
| heart (T-3399) DCM | 29426 | 30.03 | 9.1 | 18.20 | heart (T-3399) DCM | −3.09 |
| adenoid GW99-269 | 26162 | 30.31 | 7.05 | 14.10 | adenoid | |
| tonsil GW98-280 | 22582 | 29.06 | 21.74 | 43.48 | tonsil | |
| T cells PC00314 | 28453 | 29.07 | 21.64 | 43.28 | T cells | |
| PBMNC | | 32.15 | 1.35 | 1.35 | PBMNC | |
| monocyte | | 31.25 | 3.03 | 6.06 | monocyte | |
| B cells PC00665 | 28455 | 29.44 | 15.46 | 30.92 | B cells | |
| dendritic cells | 28441 | 30.62 | 5.35 | 10.70 | dendritic cells | |
| neutrophils | 28440 | 29.12 | 20.61 | 20.61 | neutrophils | |
| eosinophils | 28446 | 29.41 | 15.85 | 31.70 | eosinophils | |
| BM unstim | | 32.07 | 1.45 | 1.45 | BM unstim | |
| BM stim | treated | 30.78 | 4.65 | 4.65 | BM stim | 3.21 |
| osteo dif | treated | 31.61 | 2.19 | 2.19 | osteo dif | 1.58 |
| osteo undif | | 32.12 | 1.39 | 1.39 | osteo undif | |
| chondrocytes | | 30.4 | 6.55 | 16.38 | chondrocyt s | |
| OA Synovium IP12/01 | 29462 | 28.48 | 36.75 | 36.75 | OA Synovium | |
| OA Synovium NP10/01 | 29461 | 29.49 | 14.84 | 29.68 | OA Synovium | |
| OA Synovium NP57/00 | 28464 | 28.59 | 33.29 | 66.58 | OA Synovium | |
| RA Synovium | 28466 | 28.6 | 32.84 | 65.68 | RA Synovium | |
| NP03/01 | | | | | | |
| RA Synovium | 28467 | 28.6 | 32.95 | 65.90 | RA Synovium | |
| NP71/00 | | | | | | |
| RA Synovium | 28475 | 29.19 | 19.32 | 38.64 | RA Synovium | |
| NP45/00 | | | | | | |
| OA bone (biobank) | 29217 | 28.49 | 36.53 | 36.53 | OA bone (biobank) | |
| OA bone Sample 1 | J. Emory | 28.42 | 38.86 | 77.72 | OA bone | |
| OA bone Sample 2 | J. Emory | 28.73 | 29.22 | 58.44 | OA bone | |
| Cartilage (pool) | Normal | 29.39 | 16.21 | 32.42 | Nml Cartilage (pool) | |
| Cartilage (pool) | OA | 30.62 | 5.33 | 10.66 | OA Cartilage (pool) | −3.04 |
| PBL unifected | 28441 | 29.94 | 9.86 | 19.72 | PBL unifected | |
| PBL HIV IIIB | 28442 | 29.36 | 16.65 | 33.30 | PBL HIV IIIB | 1.69 |
| MRC5 uninfected | 29158 | 30.27 | 7.36 | 14.72 | MRC5 uninfected | |
| (100%) | | | | | (100%) | |
| MRC5 HSV strain F | 29178 | 30.73 | 4.83 | 9.66 | MRC5 HSV strain F | −1.52 |
| W12 cells | 29179 | 31.09 | 3.5 | 7.00 | W12 cells | −1.29 |
| Keratinocytes | 29180 | 30.81 | 4.51 | 9.02 | Keratinocytes | |
| B-actin control | | 27.35 | 101.64 | | | |
| genomic | | 25.51 | 531.17 | | | |
| 1.00E+05 | | 19.06 | 100000 | | | |
| 1.00E+05 | | 19.11 | 100000 | | | |
| 1.00E+04 | | 22.1 | 10000 | | | |
| 1.00E+04 | | 21.85 | 10000 | | | |
| 1.00E+03 | | 25.01 | 1000 | | | |
| 1.00E+03 | | 24.81 | 1000 | | | |
| 1.00E+02 | | 28.64 | 100 | | | |
| 1.00E+02 | | 29.11 | 100 | | | |
| 1.00E+01 | | 30.45 | 10 | | | |
| 1.00E+01 | | 30.95 | 10 | | | |
| 1.00E+00 | | 30.99 | 1 | | | |
| 1.00E+00 | | 30.95 | 1 | | | |
| NTC | | 31.42 | −1 | | | |

*lung 26 Normal has been omitted due to multiple amplification failures from that sample

Gene Name sbg1522659Siglec

| | Fold Change in Disease |
|---|---|
| | Population Relative to |
| Disease tissues | Normal |
| colon tumor | −1.19 |
| colon tumor | 2.08 |
| colon tumor | −1.14 |
| colon tumor | 1.60 |
| lung tumor | −2.33 |
| lung tumor | 6.31 |
| lung tumor | −2.13 |
| lung tumor | 2.35 |
| breast tumor | 2.10 |
| breast tumor | 6.73 |
| breast tumor | 1.39 |
| breast tumor | −1.02 |
| brain stage 5 ALZ | −1.01 |
| brain stage 5 ALZ | 2.05 |
| brain stage 5 ALZ | 1.68 |
| brain stage 5 ALZ | 1.02 |
| lung 24 COPD | −16.29 |
| lung 28 COPD | −13.42 |
| lung 23 COPD | −20.27 |
| asthmatic lung | −7.84 |
| asthmatic lung | −1.70 |
| asthmatic lung | 2.97 |
| asthmatic lung | −1.20 |
| endo VEGF | −2.35 |
| endo bFGF | −1.11 |
| heart (T-1) ischemic | −3.12 |
| heart (T-14) non-obstructive DCM | −4.81 |
| heart (T-3399) DCM | −3.09 |
| BM stim | 3.21 |
| osteo dif | 1.58 |
| OA Cartilage (pool) | −3.04 |
| PEL HIV IIIB | 1.69 |
| MRC5 HSV strain F | −1.52 |
| W12 cells | −1.29 |

Gene Name sbg1528389ITI

Moderate overall expression. This gene is expressed fairly ubiquitously in the normal samples with the highest expression seen in the whole brain, lung, thymus, and testis. The highest disease expression is seen in some of the colon, breast, and lung normal and tumor samples as well as in the asthmatic lung samples. Upregulation in 2 of 4 colon tumor samples implicates this gene in colon cancer. Upregulation in 2 of 4 asthmatic lung samples indicates a possible involvement in asthma Upregulation in the HIV-infected PBLs cells suggests that this gene may be a host factor in HIV. Upregulation in the OA cartilage pool and high expression in the RA and OA synovium samples, and the OA bone samples with corroborating high expression in the T cells, B cells, and neutrophils, dendritic cells, and the eosinophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1528389ITI | 1 and 2) | 1) | 2) | Copies | (ng) | (ng) | RNA |
| Subcutaneous | 40, 39.7 | 0 | 0.43 | 0.22 | 3.06 | 16.34 | 3.51 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 37.07, 39.08 | 2.04 | 0.62 | 1.33 | 0.61 | 81.97 | 109.02 |
| Whole Brain Clontech | 27.35, 27.52 | 634.74 | 576.15 | 605.45 | 7.24 | 6.91 | 4181.25 |
| Fetal Brain Clontech | 36.09, 37.27 | 3.64 | 1.82 | 2.73 | 0.48 | 103.95 | 283.78 |
| Cerebellum Clontech | 36.66, 36.18 | 2.6 | 3.45 | 3.03 | 2.17 | 23.04 | 69.70 |
| Cervix | 38.2, 36.11 | 1.05 | 3.6 | 2.33 | 2.42 | 20.66 | 48.04 |
| Colon | 33.09, 32.85 | 21.4 | 24.64 | 23.02 | 2.71 | 18.45 | 424.72 |
| Endometrium | 37.25, 34.99 | 1.84 | 6.99 | 4.42 | 0.73 | 68.21 | 301.16 |
| Esophagus | 37.06, 36.38 | 2.05 | 3.07 | 2.56 | 1.37 | 36.50 | 93.43 |
| Heart Clontech | 37.13, 35.89 | 1.98 | 4.1 | 3.04 | 1.32 | 37.88 | 115.15 |
| Hypothalamus | 38.14, 37.58 | 1.09 | 1.51 | 1.30 | 0.32 | 155.28 | 201.86 |
| Ileum | 34.17, 34.6 | 11.31 | 8.76 | 10.04 | 2.58 | 19.38 | 194.48 |
| Jejunum | 33.32, 32.88 | 18.73 | 24.26 | 21.50 | 6.60 | 7.58 | 162.84 |
| Kidney | 36.95, 39.47 | 2.2 | 0.49 | 1.35 | 2.12 | 23.58 | 31.72 |
| Liver | 35.02, 35.4 | 6.87 | 5.48 | 6.18 | 1.50 | 33.33 | 205.83 |
| Fetal Liver Clontech | 33.53, 33.48 | 16.53 | 17.05 | 16.79 | 10.40 | 4.81 | 80.72 |
| Lung | 34.5, 31.28 | 9.32 | 62.37 | 35.85 | 2.57 | 19.46 | 697.37 |
| Mammary Gland | 34.17, 33.53 | 11.34 | 16.56 | 13.95 | 13.00 | 3.85 | 53.65 |
| Clontech | | | | | | | |
| Myometrium | 34.52, 35.41 | 9.22 | 5.44 | 7.33 | 2.34 | 21.37 | 156.62 |
| Omentum | 35.14, 34.07 | 6.4 | 12.03 | 9.22 | 3.94 | 12.69 | 116.94 |
| Ovary | 34.02, 34.32 | 12.37 | 10.37 | 11.37 | 4.34 | 11.52 | 130.99 |
| Pancreas | 36.86, 37.21 | 2.31 | 1.88 | 2.10 | 0.81 | 61.80 | 129.48 |
| Head of Pancreas | 34.54, 35.46 | 9.09 | 5.3 | 7.20 | 1.57 | 31.85 | 229.14 |
| Parotid Gland | 32.22, 33.21 | 35.77 | 20 | 27.89 | 5.48 | 9.12 | 254.43 |
| Placenta Clontech | 33.64, 34.31 | 15.47 | 10.44 | 12.96 | 5.26 | 9.51 | 123.15 |
| Prostate | 35.95, 38.03 | 3.95 | 1.16 | 2.56 | 3.00 | 16.67 | 42.58 |
| Rectum | 36.19, 34.63 | 3.43 | 8.63 | 6.03 | 1.23 | 40.65 | 245.12 |
| Salivary Gland Clontech | 34.97, 35.39 | 7.07 | 5.52 | 6.30 | 7.31 | 6.84 | 43.06 |
| Skeletal Muscle | 36.99, 36.59 | 2.14 | 2.71 | 2.43 | 1.26 | 39.68 | 96.23 |
| Clontech | | | | | | | |
| Skin | 34.99, 34.63 | 6.97 | 8.63 | 7.80 | 1.21 | 41.32 | 322.31 |
| Small Intestine Clontech | 35.78, 36.83 | 4.37 | 2.35 | 3.36 | 0.98 | 51.07 | 171.60 |
| Spleen | 36.13, 37.09 | 3.56 | 2.02 | 2.79 | 4.92 | 10.16 | 28.35 |
| Stomach | 35.12, 34.26 | 6.47 | 10.73 | 8.60 | 2.73 | 18.32 | 157.51 |
| Testis Clontech | 35.84, 35.35 | 4.23 | 5.64 | 4.94 | 0.57 | 87.87 | 433.66 |
| Thymus Clontech | 30.97, 30.87 | 75.12 | 79.5 | 77.31 | 9.89 | 5.06 | 390.85 |
| Thyroid | 35.11, 35.5 | 6.48 | 5.15 | 5.82 | 2.77 | 18.05 | 104.96 |
| Trachea Clontech | 33.16, 33.71 | 20.51 | 14.85 | 17.68 | 9.71 | 5.15 | 91.04 |
| Urinary Bladder | 35.13, 35.13 | 6.42 | 6.42 | 6.42 | 5.47 | 9.14 | 58.68 |
| Uterus | 33.91, 33.81 | 13.18 | 14.02 | 13.60 | 5.34 | 9.36 | 127.34 |
| genomic | 24.82 | 2824.82 | | | | | |
| b-actin | 27.4 | 615.4 | | | | | |
| 1.00E+05 | 19.68 | 100000 | | | | | |
| 1.00E+05 | 19.74 | 100000 | | | | | |
| 1.00E+04 | 21.44 | 10000 | | | | | |
| 1.00E+04 | 22.63 | 10000 | | | | | |
| 1.00E+03 | 26.05 | 1000 | | | | | |
| 1.00E+03 | 25.81 | 1000 | | | | | |
| 1.00E+02 | 30.64 | 100 | | | | | |
| 1.00E+02 | 30.82 | 100 | | | | | |
| 1.00E+01 | 33.47 | 10 | | | | | |
| 1.00E+01 | 34.14 | 10 | | | | | |
| 1.00E+00 | 39.1 | 1 | | | | | |
| 1.00E+00 | 38.87 | 1 | | | | | |
| NTC | 40 | −1 | | | | | |
| NTC | 39.62 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1528389ITI | identifier) | Ct | copies | RNA | Sample | Population |
| colon normal GW98-167 | 21941 | 29.14 | 216.46 | 432.92 | colon normal | |
| colon tumor GW98-166 | 21940 | 27.54 | 550.97 | 1101.94 | colon tumor | 2.55 |
| colon normal GW98-178 | 22080 | 32.22 | 35.92 | 71.84 | colon normal | |
| colon tumor GW98-177 | 22060 | 27.74 | 488.73 | 977.46 | colon tumor | 13.61 |
| colon normal GW98-561 | 23514 | 30.58 | 93.27 | 186.54 | colon normal | |
| colon tumor GW98-560 | 23513 | 26.52 | 996.72 | 1993.44 | colon tumor | 10.69 |
| colon normal GW98-894 | 24691 | 29.1 | 221.71 | 443.42 | colon normal | |
| colon tumor GW98-893 | 24690 | 28 | 420.56 | 841.12 | colon tumor | 1.90 |
| lung normal GW98-3 | 20742 | 28.35 | 343.97 | 687.94 | lung normal | |
| lung tumor GW98-2 | 20741 | 29.86 | 141.95 | 283.90 | lung tumor | −2.42 |
| lung normal GW97-179 | 20677 | 26.22 | 1190.76 | 2381.52 | lung normal | |
| lung tumor GW97-178 | 20676 | 28.38 | 337.28 | 674.56 | lung tumor | −3.53 |
| lung normal GW98-165 | 21922 | 26.98 | 764.17 | 1528.34 | lung normal | |
| lung tumor GW98-164 | 21921 | 28.26 | 362.34 | 724.68 | lung tumor | −2.11 |
| lung normal GW98-282 | 22584 | 31.43 | 56.99 | 113.98 | lung normal | |
| lung tumor GW98-281 | 22583 | 31.03 | 72.06 | 144.12 | lung tumor | 1.26 |
| breast normal GW00-392 | 28750 | 29.1 | 221.58 | 221.58 | breast normal | |
| breast tumor GW00-391 | 28746 | 29.16 | 213.78 | 427.56 | breast tumor | 1.93 |
| breast normal GW00-413 | 28798 | 31.29 | 61.63 | 61.63 | breast normal | |
| breast tumor GW00-412 | 28797 | 30.38 | 105.24 | 210.48 | breast tumor | 3.42 |
| breast normal GW00- | 27592-95 | 30.91 | 76.93 | 76.93 | breast normal | |
| 235:238 | | | | | | |
| breast tumor GW00- | 27588-91 | 32.6 | 28.82 | 28.82 | breast tumor | −2.67 |
| 231:234 | | | | | | |
| breast normal GW98-621 | 23656 | 31.46 | 55.99 | 111.98 | breast normal | |
| breast tumor GW98-620 | 23655 | 30.46 | 100.08 | 200.16 | breast tumor | 1.79 |
| brain normal BB99-542 | 25507 | 31.13 | 67.76 | 135.52 | brain normal | |
| brain normal BB99-406 | 25509 | 30.81 | 81.84 | 163.68 | brain normal | |
| brain normal BB99-904 | 25546 | 32.62 | 28.45 | 56.90 | brain normal | |
| brain stage 5 ALZ BB99- | 25502 | 31.06 | 70.8 | 141.60 | brain stage 5 ALZ | 1.19 |
| 874 | | | | | | |
| brain stage 5 ALZ BB99- | 25503 | 30.4 | 103.5 | 207.00 | brain stage 5 ALZ | 1.74 |
| 887 | | | | | | |
| brain stage 5 ALZ BB99- | 25504 | 31.15 | 67.03 | 134.06 | brain stage 5 ALZ | 1.13 |
| 862 | | | | | | |
| brain stage 5 ALZ BB99- | 25542 | 31.38 | 58.64 | 117.28 | brain stage 5 ALZ | −1.01 |
| 927 | | | | | | |
| CT lung | normal | 29.75 | 151.95 | 303.90 | CT lung Nml | |
| lung 26 | normal | 28.26 | 361.08 | | lung 26 Nml | |
| lung 27 | normal | 31.13 | 67.84 | 67.84 | lung 27 Nml | |
| lung 24 | COPD | 30.48 | 99.14 | 99.14 | lung 24 COPD | −1.73 |
| lung 28 | COPD | 31.22 | 64.26 | 64.26 | lung 28 COPD | −2.66 |
| lung 23 | COPD | 33.41 | 17.89 | 17.89 | lung 23 COPD | −9.56 |
| lung 25 | normal | 29.87 | 141.5 | 141.50 | lung 25 Nml | |
| asthmatic lung ODO3112 | 29321 | 29.23 | 205.61 | 205.61 | asthmatic lung | 1.20 |
| asthmatic lung ODO3433 | 29323 | 29.42 | 183.77 | 367.54 | asthmatic lung | 2.15 |
| asthmatic lung ODO3397 | 29322 | 27.81 | 469.96 | 939.92 | asthmatic lung | 5.49 |
| asthmatic lung ODO4928 | 29325 | 28.16 | 382.12 | 764.24 | asthmatic lung | 4.47 |
| endo cells | control | 35.58 | 5.07 | 5.07 | endo cells | |
| endo VEGF | | 36.5 | 2.95 | 2.95 | endo VEGF | −1.72 |
| endo bFGF | | 35.2 | 6.32 | 6.32 | endo bFGF | 1.25 |
| heart Clontech | normal | 36.64 | 2.73 | 5.46 | heart | |
| heart (T-1) ischemic | 29417 | 32.68 | 27.53 | 55.06 | heart (T-1) ischemic | 10.08 |
| heart (T-14) non- | 29422 | 39.58 | 0.49 | 0.98 | heart (T-14) non- | −5.57 |
| obstructive DCM | | | | | obstructive DCM | |
| heart (T-3399) DCM | 29426 | 35.02 | 7.02 | 14.04 | heart (T-3399) DCM | 2.57 |
| adenoid GW99-269 | 26162 | 31.63 | 50.7 | 101.40 | adenoid | |
| tonsil GW98-280 | 22582 | 30.69 | 87.76 | 175.52 | tonsil | |
| T cells PC00314 | 28453 | 30.3 | 109.89 | 219.78 | T cells | |
| PBMNC | | 37.37 | 1.78 | 1.78 | PBMNC | |
| monocyte | | 34.45 | 9.8 | 19.60 | monocyte | |
| B cells PC00665 | 28455 | 31.31 | 60.88 | 121.76 | B cells | |
| dendritic cells | 28441 | 32.25 | 35.19 | 70.38 | dendritic cells | |
| neutrophils | 28440 | 32.3 | 34.36 | 34.36 | neutrophils | |
| eosinophils | 28446 | 32.85 | 24.86 | 49.72 | eosinophils | |
| BM unstim | | 37.04 | 2.16 | 2.16 | BM unstim | |
| BM stim | treated | 40 | 0 | 0.00 | BM stim | −2.16 |
| osteo dif | treated | 35.82 | 4.39 | 4.39 | osteo dif | 1.15 |
| osteo undif | | 36.06 | 3.81 | 3.81 | osteo undif | |
| chondrocytes | | 34.57 | 9.12 | 22.80 | chondrocytes | |
| OA Synovium IP12/01 | 29462 | 30.02 | 129.3 | 129.30 | OA Synovium | |
| OA Synovium NP10/01 | 29461 | 31.82 | 45.36 | 90.72 | OA Synovium | |
| OA Synovium NP57/00 | 28464 | 30.27 | 112.19 | 224.38 | OA Synovium | |
| RA Synovium | 28466 | 30.84 | 80.36 | 160.72 | RA Synovium | |
| NP03/01 | | | | | | |
| RA Synovium | 28467 | 30.23 | 114.46 | 228.92 | RA Synovium | |
| NP71/00 | | | | | | |
| RA Synovium | 28475 | 36.06 | 3.81 | 7.62 | RA Synovium | |
| NP45/00 | | | | | | |
| OA bone (biobank) | 29217 | 32.73 | 26.65 | 26.65 | OA bone (biobank) | |
| OA bone Sample 1 | J. Emory | 31.47 | 55.69 | 111.38 | OA bone | |
| OA bone Sample 2 | J. Emory | 33.85 | 13.88 | 27.76 | OA bone | |
| Cartilage (pool) | Normal | 40 | 0 | 0.00 | Nml Cartilage (pool) | |
| Cartilage (pool) | OA | 32.49 | 30.73 | 61.46 | OA Cartilage (pool) | 61.46 |
| PBL unifected | 28441 | 34.32 | 10.57 | 21.14 | PBL unifected | |
| PBL HIV IIIB | 28442 | 31.22 | 64.17 | 128.34 | PBL HIV IIIB | 6.07 |
| MRC5 uninfected | 29158 | 32.19 | 36.6 | 73.20 | MRC5 uninfected | |
| (100%) | | | | | (100%) | |
| MRC5 HSV strain F | 29178 | 32.13 | 37.92 | 75.84 | MRC5 HSV strain F | 1.04 |
| W12 cells | 29179 | 32.9 | 24.2 | 48.40 | W12 cells | −1.23 |
| Keratinocytes | 29180 | 32.54 | 29.85 | 59.70 | Keratinocytes | |
| B-actin control | | 27.27 | 643.43 | | | |
| genomic | | 26.42 | 1058.14 | | | |
| 1.00E+05 | | 19.45 | 100000 | | | |
| 1.00E+05 | | 19.73 | 100000 | | | |
| 1.00E+04 | | 22.57 | 10000 | | | |
| 1.00E+04 | | 22.68 | 10000 | | | |
| 1.00E+03 | | 25.02 | 1000 | | | |
| 1.00E+03 | | 25.15 | 1000 | | | |
| 1.00E+02 | | 28.95 | 100 | | | |
| 1.00E+02 | | 30.01 | 100 | | | |
| 1.00E+01 | | 33.54 | 10 | | | |
| 1.00E+01 | | 37.65 | 10 | | | |
| 1.00E+00 | | 38.77 | 1 | | | |
| 1.00E+00 | | 40 | 0 | | | |
| NTC | | 38.96 | −1 | | | |

*lung 26 Normal has been omitted due to multiple amplification failures from that sample

Gene Name sbg1528389ITI

| | Fold Change in Disease |
|---|---|
| | Population Relative to |
| Disease tissues | Normal |
| colon tumor | 2.55 |
| colon tumor | 13.61 |
| colon tumor | 10.69 |
| colon tumor | 1.90 |
| lung tumor | −2.42 |
| lung tumor | −3.53 |
| lung tumor | −2.11 |
| lung tumor | 1.26 |
| breast tumor | 1.93 |
| breast tumor | 3.42 |
| breast tumor | −2.67 |
| breast tumor | 1.79 |
| brain stage 5 ALZ | 1.19 |
| brain stage 5 ALZ | 1.74 |
| brain stage 5 ALZ | 1.13 |
| brain stage 5 ALZ | −1.01 |
| lung 24 COPD | −1.73 |
| lung 28 COPD | −2.66 |
| lung 23 COPD | −9.56 |
| asthmatic lung | 1.20 |
| asthmatic lung | 2.15 |
| asthmatic lung | 5.49 |
| asthmatic lung | 4.47 |
| endo VEGF | −1.72 |
| endo bFGF | 1.25 |
| heart (T-1) ischemic | 10.08 |
| heart (T-14) non-obstructive DCM | −5.57 |
| heart (T-3399) DCM | 2.57 |
| BM stim | −2.16 |
| osteo dif | 1.15 |
| OA Cartilage (pool) | 61.46 |
| PBL HIV IIIB | 6.07 |
| MRC5 HSV strain F | 1.04 |
| W12 cells | −1.23 |

Gene Name sbg15269641SLR

Moderate overall expression. The highest normal expression is seen in the subcutaneous adipocytes, whole brain, fetal brain, liver, thymus, and fetal liver. Additional expression is seen in the colon and rectum. The highest disease expression is seen in the breast, colon, and lung normal and tumor samples as well as in the dendritic cell, the eosinophils, and the uninfected PBLS. Upregulation in 1 of 4 breast tumor samples implicates this gene in breast cancer. Upregulation in 3 of 4 Alzheimer's brain samples suggests an involvement in Alzheimer's disease. Upregulation in 4 of 4 asthmatic lung samples indicates a possible involvement in asthma. Upregulated in the stimulated bone marrow and the differentiated osteoblasts. Downregulation in the HIV-infected PBLs and the HSV-infected MRC5 cells suggests that this gene may be a host factor in HIV and HSV. Downregulation in the W12 cells suggests that this gene may be a host factor in HPV. High expression in the RA and OA synovium samples, the OA bone samples, and the chondrocytes with corroborating high expression in the T cells, B cells, neutrophils, dendritic cells, and the eosinophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1526964ISLR | 1 and 2) | 1) | 2) | Copies | (ng) | (ng) | RNA |
| Subcutaneous | 32.03, 31.87 | 86.32 | 94.34 | 90.33 | 3.06 | 16.34 | 1475.98 |
| Adipocytes Zenbio | | | | | | | |
| Subcutaneous Adipose | 36.36, 37.6 | 7.63 | 3.8 | 5.72 | 0.96 | 52.36 | 299.21 |
| Zenbio | | | | | | | |
| Adrenal Gland | 40, 40 | 0 | 0 | 0.00 | 0.61 | 81.97 | 0.00 |
| Clontech | | | | | | | |
| Whole Brain Clontech | 28.56, 28.16 | 602.41 | 753.27 | 677.84 | 7.24 | 6.91 | 4681.22 |
| Fetal Brain Clontech | 34.51, 32.76 | 21.5 | 57.2 | 39.40 | 0.48 | 103.95 | 4095.11 |
| Cerebellum Clontech | 37.92, 40 | 3.18 | 0 | 1.59 | 2.17 | 23.04 | 36.64 |
| Cervix | 40, 36.56 | 0 | 6.83 | 3.42 | 2.42 | 20.66 | 70.56 |
| Colon | 34.23, 34.51 | 25.09 | 21.49 | 23.29 | 2.71 | 18.45 | 429.70 |
| Endometrium | 36.12, 36.1 | 8.71 | 8.83 | 8.77 | 0.73 | 68.21 | 598.23 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 35.01, 40 | 16.23 | 0 | 8.12 | 2.58 | 19.38 | 157.27 |
| Jejunum | 33.6, 33.32 | 35.76 | 41.84 | 38.80 | 6.60 | 7.58 | 293.94 |
| Kidney | 40, 40 | 0 | 0 | 0.00 | 2.12 | 23.58 | 0.00 |
| Liver | 33.77, 34.04 | 32.62 | 27.91 | 30.27 | 1.50 | 33.33 | 1008.83 |
| Fetal Liver Clontech | 29.78, 29.53 | 303.66 | 349.23 | 326.45 | 10.40 | 4.81 | 1569.45 |
| Lung | 36.17, 35.56 | 8.51 | 11.95 | 10.23 | 2.57 | 19.46 | 199.03 |
| Mammary Gland | 32.26, 31.59 | 75.95 | 110.01 | 92.98 | 13.00 | 3.85 | 357.62 |
| Clontech | | | | | | | |
| Myometrium | 34.73, 40 | 19.02 | 0 | 9.51 | 2.34 | 21.37 | 203.21 |
| Omentum | 40, 40 | 0 | 0 | 0.00 | 3.94 | 12.69 | 0.00 |
| Ovary | 33.48, 33.38 | 38.36 | 40.55 | 39.46 | 4.34 | 11.52 | 454.55 |
| Pancreas | 40, 34.09 | 0 | 27.28 | 13.64 | 0.81 | 61.80 | 843.02 |
| Head of Pancreas | 40, 40 | 0 | 0 | 0.00 | 1.57 | 31.85 | 0.00 |
| Parotid Gland | 33, 34.94 | 49.96 | 16.95 | 33.46 | 5.48 | 9.12 | 305.25 |
| Placenta Clontech | 36, 35.49 | 9.33 | 12.42 | 10.88 | 5.26 | 9.51 | 103.37 |
| Prostate | 35.4, 40 | 13.09 | 0 | 6.55 | 3.00 | 16.67 | 109.08 |
| Rectum | 34.06, 35.27 | 27.61 | 14.02 | 20.82 | 1.23 | 40.65 | 846.14 |
| Salivary Gland | 33.4, 33.53 | 39.97 | 37.31 | 38.64 | 7.31 | 6.84 | 264.30 |
| Clontech | | | | | | | |
| Skeletal Muscle | 40, 40 | 0 | 0 | 0.00 | 1.26 | 39.68 | 0.00 |
| Clontech | | | | | | | |
| Skin | 36.6, 40 | 6.69 | 0 | 3.35 | 1.21 | 41.32 | 138.22 |
| Small Intestine | 38.85, 40 | 1.89 | 0 | 0.95 | 0.98 | 51.07 | 48.26 |
| Clontech | | | | | | | |
| Spleen | 40, 34.46 | 0 | 22.14 | 11.07 | 4.92 | 10.16 | 112.50 |
| Stomach | 36.86, 35.09 | 5.78 | 15.52 | 10.65 | 2.73 | 18.32 | 195.05 |
| Testis Clontech | 40, 35.99 | 0 | 9.38 | 4.69 | 0.57 | 87.87 | 412.13 |
| Thymus Clontech | 31.03, 31.12 | 151.08 | 143.44 | 147.26 | 9.89 | 5.06 | 744.49 |
| Thyroid | 40, 40 | 0 | 0 | 0.00 | 2.77 | 18.05 | 0.00 |
| Trachea Clontech | 33.62, 34.06 | 35.48 | 27.61 | 31.55 | 9.71 | 5.15 | 162.44 |
| Urinary Bladder | 35.3, 36.69 | 13.81 | 6.35 | 10.08 | 5.47 | 9.14 | 92.14 |
| Uterus | 34.52, 33.14 | 21.39 | 46.31 | 33.85 | 5.34 | 9.36 | 316.95 |
| genomic | 27.07 | 1382.37 | | | | | |
| b-actin | 27.91 | 866.2 | | | | | |
| 1.00E+05 | 19.67 | 100000 | | | | | |
| 1.00E+05 | 19.81 | 100000 | | | | | |
| 1.00E+04 | 23.26 | 10000 | | | | | |
| 1.00E+04 | 23.26 | 10000 | | | | | |
| 1.00E+03 | 27.11 | 1000 | | | | | |
| 1.00E+03 | 27.29 | 1000 | | | | | |
| 1.00E+02 | 31.65 | 100 | | | | | |
| 1.00E+02 | 32.86 | 100 | | | | | |
| 1.00E+01 | 35.52 | 10 | | | | | |
| 1.00E+01 | 36.09 | 10 | | | | | |
| 1.00E+00 | 40 | 0 | | | | | |
| 1.00E+00 | 40 | 1 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1526964ISLR | identifier) | Ct | copies | RNA | Sample | Population |
| colon normal GW98-167 | 21941 | 26.36 | 1672.61 | 3345.22 | colon normal | |
| colon tumor GW98-166 | 21940 | 27.67 | 795.52 | 1591.04 | colon tumor | −2.10 |
| colon normal GW98-178 | 22080 | 28.28 | 561.07 | 1122.14 | colon normal | |
| colon tumor GW98-177 | 22060 | 28.49 | 499.37 | 998.74 | colon tumor | −1.12 |
| colon normal GW98-561 | 23514 | 28.19 | 592.91 | 1185.82 | colon normal | |
| colon tumor GW98-560 | 23513 | 27.14 | 1073.47 | 2146.94 | colon tumor | 1.81 |
| colon normal GW98-894 | 24691 | 27.48 | 884.72 | 1769.44 | colon normal | |
| colon tumor GW98-893 | 24690 | 27.3 | 984.57 | 1969.14 | colon tumor | 1.11 |
| lung normal GW98-3 | 20742 | 27.32 | 970.01 | 1940.02 | lung normal | |
| lung tumor GW98-2 | 20741 | 28.11 | 618.42 | 1236.84 | lung tumor | −1.57 |
| lung normal GW97-179 | 20677 | 27.76 | 757.07 | 1514.14 | lung normal | |
| lung tumor GW97-178 | 20676 | 27.82 | 731.21 | 1462.42 | lung tumor | −1.04 |
| lung normal GW98-165 | 21922 | 26.55 | 1505.27 | 3010.54 | lung normal | |
| lung tumor GW98-164 | 21921 | 28.86 | 403.81 | 807.62 | lung tumor | −3.73 |
| lung normal GW98-282 | 22584 | 29.13 | 347.08 | 694.16 | lung normal | |
| lung tumor GW98-281 | 22583 | 29.38 | 300.54 | 601.08 | lung tumor | −1.15 |
| breast normal GW00-392 | 28750 | 29.57 | 270.38 | 270.38 | breast normal | |
| breast tumor GW00-391 | 28746 | 29.5 | 281.83 | 563.66 | breast tumor | 2.08 |
| breast normal GW00-413 | 28798 | 28.16 | 602.86 | 602.86 | breast normal | |
| breast tumor GW00-412 | 28797 | 28.12 | 614.3 | 1228.60 | breast tumor | 2.04 |
| breast normal GW00- | 27592-95 | 30.02 | 208.6 | 208.60 | breast normal | |
| 235:238 | | | | | | |
| breast tumor GW00- | 27588-91 | 29.56 | 271.58 | 271.58 | breast tumor | 1.30 |
| 231:234 | | | | | | |
| breast normal GW98-621 | 23656 | 29.07 | 359.48 | 718.96 | breast normal | |
| breast tumor GW98-620 | 23655 | 27.48 | 887.97 | 1775.94 | breast tumor | 2.47 |
| brain normal BB99-542 | 25507 | 31.18 | 108.39 | 216.78 | brain normal | |
| brain normal BB99-406 | 25509 | 32.07 | 65.03 | 130.06 | brain normal | |
| brain normal BB99-904 | 25546 | 31.93 | 70.43 | 140.86 | brain normal | |
| brain stage 5 ALZ BB99- | 25502 | 30.08 | 202.54 | 405.08 | brain stage 5 ALZ | 2.49 |
| 874 | | | | | | |
| brain stage 5 ALZ BB99- | 25503 | 28.53 | 486.51 | 973.02 | brain stage 5 ALZ | 5.99 |
| 887 | | | | | | |
| brain stage 5 ALZ BB99- | 25504 | 29.63 | 261.11 | 522.22 | brain stage 5 ALZ | 3.21 |
| 862 | | | | | | |
| brain stage 5 ALZ BB99- | 25542 | 31.07 | 114.88 | 229.76 | brain stage 5 ALZ | 1.41 |
| 927 | | | | | | |
| CT lung | normal | 29.16 | 341.04 | 682.08 | CT lung Nml | |
| lung 26 | normal | 29.13 | 347.67 | | lung 26 Nml | |
| lung 27 | normal | 34.08 | 20.78 | 20.78 | lung 27 Nml | |
| lung 24 | COPD | 34.17 | 19.77 | 19.77 | lung 24 COPD | −12.19 |
| lung 28 | COPD | 33.07 | 36.85 | 36.85 | lung 28 COPD | −6.54 |
| lung 23 | COPD | 36.23 | 6.12 | 6.12 | lung 23 COPD | −39.37 |
| lung 25 | normal | 34.16 | 19.9 | 19.90 | lung 25 Nml | |
| asthmatic lung ODO3112 | 29321 | 28.42 | 519.08 | 519.08 | asthmatic lung | 2.15 |
| asthmatic lung ODO3433 | 29323 | 28.73 | 435.93 | 871.86 | asthmatic lung | 3.62 |
| asthmatic lung ODO3397 | 29322 | 28.45 | 510.25 | 1020.50 | asthmatic lung | 4.24 |
| asthmatic lung ODO4928 | 29325 | 28.56 | 480.73 | 961.46 | asthmatic lung | 3.99 |
| endo cells | control | 29.91 | 223.16 | 223.16 | endo cells | |
| endo VEGF | | 30.84 | 131.52 | 131.52 | endo VEGF | −1.70 |
| endo bFGF | | 30.69 | 143.15 | 143.15 | endo bFGF | −1.56 |
| heart Clontech | normal | 30.26 | 182.88 | 365.76 | heart | |
| heart (T-1)ischemic | 29417 | 29.44 | 290.65 | 581.30 | heart (T-1) | 1.59 |
| | | | | | ischemic | |
| heart (T-14) non- | 29422 | 30.36 | 172.66 | 345.32 | heart (T-14) non- | −1.06 |
| obstructive DCM | | | | | obstructive DCM | |
| heart (T-3399) DCM | 29426 | 29.37 | 303.23 | 606.46 | heart (T-3399) | 1.66 |
| | | | | | DCM | |
| adenoid GW99-269 | 26162 | 31.77 | 77.36 | 154.72 | adenoid | |
| tonsil GW98-280 | 22582 | 31.05 | 116.19 | 232.38 | tonsil | |
| T cells PC00314 | 28453 | 31.07 | 115.05 | 230.10 | T cells | |
| PBMNC | | 35.92 | 7.29 | 7.29 | PBMNC | |
| monocyte | | 36.32 | 5.82 | 11.64 | monocyte | |
| B cells PC00665 | 28455 | 29.22 | 330.33 | 660.66 | B cells | |
| dendritic cells | 28441 | 28.3 | 555.32 | 1110.64 | dendritic cells | |
| neutrophils | 28440 | 29.72 | 248.67 | 248.67 | neutrophils | |
| eosinophils | 28446 | 28.45 | 510.81 | 1021.62 | eosinophils | |
| BM unstim | | 33.48 | 29.26 | 29.26 | BM unstim | |
| BM stim | treated | 31.14 | 110.68 | 110.68 | BM stim | 3.78 |
| osteo dif | treated | 31.61 | 84.86 | 84.86 | osteo dif | 6.03 |
| osteo undif | | 34.77 | 14.07 | 14.07 | osteo undif | |
| chondrocytes | | 29.75 | 244.35 | 610.88 | chondrocytes | |
| OA Synovium IP12/01 | 29462 | 28.4 | 525.46 | 525.46 | OA Synovium | |
| OA Synovium NP10/01 | 29461 | 30.38 | 170.32 | 340.64 | OA Synovium | |
| OA Synovium NP57/00 | 28464 | 29.66 | 256.61 | 513.22 | OA Synovium | |
| RA Synovium | 28466 | 29.39 | 298.69 | 597.38 | RA Synovium | |
| NP03/01 | | | | | | |
| RA Synovium | 28467 | 29.23 | 327.25 | 654.50 | RA Synovium | |
| NP71/00 | | | | | | |
| RA Synovium | 28475 | 28.88 | 400.22 | 800.44 | RA Synovium | |
| NP45/00 | | | | | | |
| OA bone (biobank) | 29217 | 29.38 | 301.04 | 301.04 | OA bone (biobank) | |
| OA bone Sample 1 | J. Emory | 28.96 | 381.28 | 762.56 | OA bone | |
| OA bone Sample 2 | J. Emory | 30.39 | 168.95 | 337.90 | OA bone | |
| Cartilage (pool) | Normal | 30.48 | 161.19 | 322.38 | Nml Cartilage | |
| | | | | | (pool) | |
| Cartilage (pool) | OA | 30.72 | 140.64 | 281.28 | OA Cartilage | −1.15 |
| | | | | | (pool) | |
| PBL unifected | 28441 | 27.63 | 815.72 | 1631.44 | PBL unifected | |
| PBL HIV IIIB | 28442 | 29.29 | 315.83 | 631.66 | PBL HIV IIIB | −2.58 |
| MRC5 uninfected | 29158 | 30.65 | 145.99 | 291.98 | MRC5 uninfected | |
| (100%) | | | | | (100%) | |
| MRC5 HSV strain F | 29178 | 36.19 | 6.25 | 12.50 | MRC5 HSV strain F | −23.36 |
| W12 cells | 29179 | 31.18 | 107.97 | 215.94 | W12 cells | −5.27 |
| Keratinocytes | 29180 | 28.26 | 568.92 | 1137.84 | Keratinocytes | |
| B-actin control | | 27.63 | 814.59 | | | |
| genomic | | 27.4 | 929.42 | | | |
| 1.00E+05 | | 19.18 | 100000 | | | |
| 1.00E+05 | | 19.36 | 100000 | | | |
| 1.00E+04 | | 23.05 | 10000 | | | |
| 1.00E+04 | | 22.98 | 10000 | | | |
| 1.00E+03 | | 27.04 | 1000 | | | |
| 1.00E+03 | | 26.85 | 1000 | | | |
| 1.00E+02 | | 32.16 | 100 | | | |
| 1.00E+02 | | 32.19 | 100 | | | |
| 1.00E+01 | | 34.5 | 10 | | | |
| 1.00E+01 | | 40 | 10 | | | |
| 1.00E+00 | | 40 | 0 | | | |
| 1.00E+00 | | 40 | 1 | | | |
| NTC | | 40 | 0 | | | |

*lung 26 Normal has been omitted due to multiple amplification failures from that sample

Gene Name sbg15269641SLR

| | Fold Change in Disease |
|---|---|
| | Population Relative to |
| Disease tissues | Normal |
| colon tumor | −2.10 |
| colon tumor | −1.12 |
| colon tumor | 1.81 |
| colon tumor | 1.11 |
| lung tumor | −1.57 |
| lung tumor | −1.04 |
| lung tumor | −3.73 |
| lung tumor | −1.15 |
| breast tumor | 2.08 |
| breast tumor | 2.04 |
| breast tumor | 1.30 |
| breast tumor | 2.47 |
| brain stage 5 ALZ | 2.49 |
| brain stage 5 ALZ | 5.99 |
| brain stage 5 ALZ | 3.21 |
| brain stage 5 ALZ | 1.41 |
| lung 24 COPD | −12.19 |
| lung 28 COPD | −6.54 |
| lung 23 COPD | −39.37 |
| asthmatic lung | 2.15 |
| asthmatic lung | 3.62 |
| asthmatic lung | 4.24 |
| asthmatic lung | 3.99 |
| endo VEGF | −1.70 |
| endo bFGF | −1.56 |
| heart (T-1) ischemic | 1.59 |
| heart (T-14) non-obstructive DCM | −1.06 |
| heart (T-3399) DCM | 1.66 |
| BM stim | 3.78 |
| osteo dif | 6.03 |
| OA Cartilage (pool) | −1.15 |
| PBL HIV IIIB | −2.58 |
| MRC5 HSV strain F | −23.36 |
| W12 cells | −5.27 |

Gene Name sbg1539439TSPK

Moderate to low overall expression. The highest normal expression is seen in the whole brain, testis, endometrium, and trachea. The highest disease expression is seen in one of the lung tumor samples, one of the breast tumor samples, and one of the normal breast samples. Upregulation in 1 of 4 colon tumor samples, 1 of 4 lung tumor samples, and 1 of 4 breast tumor samples implicates this gene in cancers of the colon, lung, and breast. Upregulation in 1 of 4 Alzheimer's brain samples suggests an involvement in Alzheimer's disease. Downregulation in the VEGF and bFGF-treated endothelial cells suggests an involvement in angiogenesis. Upregulation in 2 of 3 disease heart samples implies an involvement in cardiovascular diseases such as obstructive DCM and ischemia Downregulated in the differentiated osteoblasts. Downregulation in the HSV-infected MRC5 cells suggests that this gene may be a host factor in HSV.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1539439TSPK | 1 and 2) | 1) | 2) | Copies | (ng) | (ng) | RNA |
| Subcutaneous | 40, 36.02 | 0 | 4.84 | 2.42 | 3.06 | 16.34 | 39.54 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 36.66, 40 | 3.29 | 0 | 1.65 | 0.61 | 81.97 | 134.84 |
| Whole Brain Clontech | 30.21, 30.36 | 157.24 | 143.95 | 150.60 | 7.24 | 6.91 | 1040.02 |
| Fetal Brain Clontech | 36.07, 40 | 4.69 | 0 | 2.35 | 0.48 | 103.95 | 243.76 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 35.92, 35.85 | 5.15 | 5.36 | 5.26 | 2.71 | 18.45 | 96.96 |
| Endometrium | 35.96, 35.78 | 5.03 | 5.59 | 5.31 | 0.73 | 68.21 | 362.21 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 34.85, 33.91 | 9.76 | 17.17 | 13.47 | 6.60 | 7.58 | 102.01 |
| Kidney | 40, 40 | 0 | 0 | 0.00 | 2.12 | 23.58 | 0.00 |
| Liver | 40, 40 | 0 | 0 | 0.00 | 1.50 | 33.33 | 0.00 |
| Fetal Liver Clontech | 33.25, 33.41 | 25.49 | 23.06 | 24.28 | 10.40 | 4.81 | 116.71 |
| Lung | 40, 33.18 | 0 | 26.53 | 13.27 | 2.57 | 19.46 | 258.07 |
| Mammary Gland | 34.09, 32.82 | 15.36 | 32.98 | 24.17 | 13.00 | 3.85 | 92.96 |
| Clontech | | | | | | | |
| Myometrium | 40, 37.47 | 0 | 2.02 | 1.01 | 2.34 | 21.37 | 21.58 |
| Omentum | 40, 40 | 0 | 0 | 0.00 | 3.94 | 12.69 | 0.00 |
| Ovary | 35.55, 34.85 | 6.4 | 9.78 | 8.09 | 4.34 | 11.52 | 93.20 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 34.15, 34.78 | 14.8 | 10.18 | 12.49 | 5.48 | 9.12 | 113.96 |
| Placenta Clontech | 40, 36.41 | 0 | 3.84 | 1.92 | 5.26 | 9.51 | 18.25 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland Clontech | 40, 35.17 | 0 | 8.04 | 4.02 | 7.31 | 6.84 | 27.50 |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 40, 35.97 | 0 | 4.99 | 2.50 | 4.92 | 10.16 | 25.36 |
| Stomach | 40, 35.78 | 0 | 5.59 | 2.80 | 2.73 | 18.32 | 51.19 |
| Testis Clontech | 40, 33.83 | 0 | 17.92 | 8.96 | 0.57 | 87.87 | 787.35 |
| Thymus Clontech | 33.81, 32.94 | 18.15 | 30.59 | 24.37 | 9.89 | 5.06 | 123.21 |
| Thyroid | 40, 40 | 0 | 0 | 0.00 | 2.77 | 18.05 | 0.00 |
| Trachea Clontech | 32.09, 31.16 | 50.98 | 89.2 | 70.09 | 9.71 | 5.15 | 360.92 |
| Urinary Bladder | 40, 40 | 0 | 0 | 0.00 | 5.47 | 9.14 | 0.00 |
| Uterus | 35.73, 34.51 | 5.74 | 11.97 | 8.86 | 5.34 | 9.36 | 82.91 |
| genomic | 27.01 | 1071.32 | | | | | |
| b-actin | 27.61 | 745.98 | | | | | |
| 1.00E+05 | 19.6 | 100000 | | | | | |
| 1.00E+05 | 19.43 | 100000 | | | | | |
| 1.00E+04 | 23.05 | 10000 | | | | | |
| 1.00E+04 | 23.03 | 10000 | | | | | |
| 1.00E+03 | 26.8 | 1000 | | | | | |
| 1.00E+03 | 26.78 | 1000 | | | | | |
| 1.00E+02 | 31.67 | 100 | | | | | |
| 1.00E+02 | 32.36 | 100 | | | | | |
| 1.00E+01 | 33.69 | 10 | | | | | |
| 1.00E+01 | 40 | 0 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1539439TSPK | identifier) | Ct | copies | RNA | Sample | Population |
| colon normal GW98-167 | 21941 | 33.02 | 44.92 | 89.84 | colon normal | |
| colon tumor GW98-166 | 21940 | 30.69 | 149.66 | 299.32 | colon tumor | 3.33 |
| colon normal GW98-178 | 22080 | 40 | 0 | 0.00 | colon normal | |
| colon tumor GW98-177 | 22060 | 32.11 | 71.99 | 143.98 | colon tumor | 143.98 |
| colon normal GW98-561 | 23514 | 33.18 | 41.4 | 82.80 | colon normal | |
| colon tumor GW98-560 | 23513 | 33.52 | 34.64 | 69.28 | colon tumor | −1.20 |
| colon normal GW98-894 | 24691 | 33.58 | 33.62 | 67.24 | colon normal | |
| colon tumor GW98-893 | 24690 | 31.29 | 109.68 | 219.36 | colon tumor | 3.26 |
| lung normal GW98-3 | 20742 | 31.18 | 116.02 | 232.04 | lung normal | |
| lung tumor GW98-2 | 20741 | 31.65 | 90.98 | 181.96 | lung tumor | −1.28 |
| lung normal GW97-179 | 20677 | 31.85 | 82.35 | 164.70 | lung normal | |
| lung tumor GW97-178 | 20676 | 31.88 | 81.04 | 162.08 | lung tumor | −1.02 |
| lung normal GW98-165 | 21922 | 30.93 | 132.27 | 264.54 | lung normal | |
| lung tumor GW98-164 | 21921 | 27.53 | 762.99 | 1525.98 | lung tumor | 5.77 |
| lung normal GW98-282 | 22584 | 29.58 | 265.31 | 530.62 | lung normal | |
| lung tumor GW98-281 | 22583 | 30.27 | 185.74 | 371.48 | lung tumor | −1.43 |
| breast normal GW00-392 | 28750 | 29.47 | 281.23 | 281.23 | breast normal | |
| breast tumor GW00-391 | 28746 | 28.91 | 374.08 | 748.16 | breast tumor | 2.66 |
| breast normal GW00-413 | 28798 | 31.18 | 115.93 | 115.93 | breast normal | |
| breast tumor GW00-412 | 28797 | 28.24 | 530.48 | 1060.96 | breast tumor | 9.15 |
| breast normal GW00- | 27592-95 | 31.64 | 91.39 | 91.39 | breast normal | |
| 235:238 | | | | | | |
| breast tumor GW00- | 27588-91 | 29.12 | 335.78 | 335.78 | breast tumor | 3.67 |
| 231:234 | | | | | | |
| breast normal GW98-621 | 23656 | 28.12 | 564.44 | 1128.88 | breast normal | |
| breast tumor GW98-620 | 23655 | 32.45 | 60.32 | 120.64 | breast tumor | −9.36 |
| brain normal BB99-542 | 25507 | 31.13 | 119.34 | 238.68 | brain normal | |
| brain normal BB99-406 | 25509 | 33.22 | 40.62 | 81.24 | brain normal | |
| brain normal BB99-904 | 25546 | 32.19 | 69.14 | 138.28 | brain normal | |
| brain stage 5 ALZ BB99- | 25502 | 31.35 | 106.38 | 212.76 | brain stage 5 ALZ | 1.39 |
| 874 | | | | | | |
| brain stage 5 ALZ BB99- | 25503 | 29.76 | 241.33 | 482.66 | brain stage 5 ALZ | 3.16 |
| 887 | | | | | | |
| brain stage 5 ALZ BB99- | 25504 | 31.08 | 122.52 | 245.04 | brain stage 5 ALZ | 1.60 |
| 862 | | | | | | |
| brain stage 5 ALZ BB99- | 25542 | 31.16 | 117.27 | 234.54 | brain stage 5 ALZ | 1.54 |
| 927 | | | | | | |
| CT lung | normal | 32.4 | 61.98 | 123.96 | CT lung Nml | |
| lung 26 | normal | 29.22 | 319.73 | | lung 26 Nml | |
| lung 27 | normal | 35.13 | 15.13 | 15.13 | lung 27 Nml | |
| lung 24 | COPD | 40 | 0 | 0.00 | lung 24 COPD | −46.36 |
| lung 28 | COPD | 40 | 0 | 0.00 | lung 28 COPD | −46.36 |
| lung 23 | COPD | 40 | 0 | 0.00 | lung 23 COPD | −46.36 |
| lung 25 | normal | 40 | 0 | 0.00 | lung 25 Nml | |
| asthmatic lung ODO3112 | 29321 | 33.11 | 42.85 | 42.85 | asthmatic lung | −1.08 |
| asthmatic lung ODO3433 | 29323 | 32.67 | 53.81 | 107.62 | asthmatic lung | 2.32 |
| asthmatic lung ODO3397 | 29322 | 32.05 | 74.04 | 148.08 | asthmatic lung | 3.19 |
| asthmatic lung ODO4928 | 29325 | 32.44 | 60.75 | 121.50 | asthmatic lung | 2.62 |
| endo cells | control | 34.69 | 18.99 | 18.99 | endo cells | |
| endo VEGF | | 40 | 0 | 0.00 | endo VEGF | −18.99 |
| endo bFGF | | 40 | 0 | 0.00 | endo bFGF | −18.99 |
| heart Clontech | normal | 33.48 | 35.46 | 70.92 | heart | |
| heart (T-1) ischemic | 29417 | 31.11 | 120.31 | 240.62 | heart (T-1) ischemic | 3.39 |
| heart (T-14) non- | 29422 | 33.38 | 37.23 | 74.46 | heart (T-14) non- | 1.05 |
| obstructive DCM | | | | | obstructive DCM | |
| heart (T-3399) DCM | 29426 | 31.22 | 113.98 | 227.96 | heart (T-3399) DCM | 3.21 |
| adenoid GW99-269 | 26162 | 34.45 | 21.49 | 42.98 | adenoid | |
| tonsil GW98-280 | 22582 | 32.46 | 60.05 | 120.10 | tonsil | |
| T cells PC00314 | 28453 | 37.71 | 3.98 | 7.96 | T cells | |
| PBMNC | | 40 | 0 | 0.00 | PBMNC | |
| monocyte | | 40 | 0 | 0.00 | monocyte | |
| B cells PC00665 | 28455 | 40 | 0 | 0.00 | B cells | |
| dendritic cells | 28441 | 40 | 0 | 0.00 | dendritic cells | |
| neutrophils | 28440 | 40 | 0 | 0.00 | neutrophils | |
| eosinophils | 28446 | 40 | 0 | 0.00 | eosinophils | |
| BM unstim | | 40 | 0 | 0.00 | BM unstim | |
| BM stim | treated | 40 | 0 | 0.00 | BM stim | 0.00 |
| osteo dif | treated | 40 | 0 | 0.00 | osteo dif | −9.66 |
| osteo undif | | 36 | 9.66 | 9.66 | osteo undif | |
| chondrocytes | | 35.21 | 14.53 | 36.33 | chondrocytes | |
| OA Synovium IP12/01 | 29462 | 29.83 | 232.79 | 232.79 | OA Synovium | |
| OA Synovium NP10/01 | 29461 | 34.09 | 25.81 | 51.62 | OA Synovium | |
| OA Synovium NP57/00 | 28464 | 30.77 | 143.53 | 287.06 | OA Synovium | |
| RA Synovium | 28466 | 32.4 | 61.91 | 123.82 | RA Synovium | |
| NP03/01 | | | | | | |
| RA Synovium | 28467 | 30.44 | 170.17 | 340.34 | RA Synovium | |
| NP71/00 | | | | | | |
| RA Synovium | 28475 | 31.75 | 86.5 | 173.00 | RA Synovium | |
| NP45/00 | | | | | | |
| OA bone (biobank) | 29217 | 33.41 | 36.7 | 36.70 | OA bone (biobank) | |
| OA bone Sample 1 | J. Emory | 31.82 | 83.64 | 167.28 | OA bone | |
| OA bone Sample 2 | J. Emory | 33.54 | 34.41 | 68.82 | OA bone | |
| Cartilage (pool) | Normal | 31.35 | 106.16 | 212.32 | Nml Cartilage (pool) | |
| Cartilage (pool) | OA | 34.25 | 23.76 | 47.52 | OA Cartilage (pool) | −4.47 |
| PBL unifected | 28441 | 34.16 | 24.9 | 49.80 | PBL unifected | |
| PBL HIV IIIB | 28442 | 35.26 | 14.17 | 28.34 | PBL HIV IIIB | −1.76 |
| MRC5 uninfected | 29158 | 30.16 | 196.24 | 392.48 | MRC5 uninfected | |
| (100%) | | | | | (100%) | |
| MRC5 HSV strain F | 29178 | 32.39 | 62.07 | 124.14 | MRC5 HSV strain F | −3.16 |
| W12 cells | 29179 | 29.09 | 342.35 | 684.70 | W12 cells | 1.41 |
| Keratinocytes | 29180 | 29.76 | 242.11 | 484.22 | Keratinocytes | |
| B-actin control | | 27.19 | 912.41 | | | |
| genomic | | 26.06 | 1629.6 | | | |
| 1.00E+05 | | 18.96 | 100000 | | | |
| 1.00E+05 | | 19 | 100000 | | | |
| 1.00E+04 | | 22.27 | 10000 | | | |
| 1.00E+04 | | 22.05 | 10000 | | | |
| 1.00E+03 | | 25.89 | 1000 | | | |
| 1.00E+03 | | 26.06 | 1000 | | | |
| 1.00E+02 | | 31.08 | 100 | | | |
| 1.00E+02 | | 31.14 | 100 | | | |
| 1.00E+01 | | 40 | 0 | | | |
| 1.00E+01 | | 37.69 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1539439TSPK

| | Fold Change in Disease |
|---|---|
| | Population Relative to |
| Disease tissues | Normal |
| colon tumor | 3.33 |
| colon tumor | 143.98 |
| colon tumor | −1.20 |
| colon tumor | 3.26 |
| lung tumor | −1.28 |
| lung tumor | −1.02 |
| lung tumor | 5.77 |
| lung tumor | −1.43 |
| breast tumor | 2.66 |
| breast tumor | 9.15 |
| breast tumor | 3.67 |
| breast tumor | −9.36 |
| brain stage 5 ALZ | 1.39 |
| brain stage 5 ALZ | 3.16 |
| brain stage 5 ALZ | 1.60 |
| brain stage 5 ALZ | 1.54 |
| lung 24 COPD | −46.36 |
| lung 28 COPD | −46.36 |
| lung 23 COPD | −46.36 |
| asthmatic lung | −1.08 |
| asthmatic lung | 2.32 |
| asthmatic lung | 3.19 |
| asthmatic lung | 2.62 |
| endo VEGF | −18.99 |
| endo bFGF | −18.99 |
| heart (T-1) ischemic | 3.39 |
| heart (T-14) non-obstructive DCM | 1.05 |
| heart (T-3399) DCM | 3.21 |
| BM stim | 0.00 |
| osteo dif | −9.66 |
| OA Cartilage (pool) | −4.47 |
| PBL HIV IIIB | −1.76 |
| MRC5 HSV strain F | −3.16 |
| W12 cells | 1.41 |

Gene Name sbg1571349lipase

Highly specific overall expression. The highest normal expression is seen in the whole brain, colon, and rectum with additional high expression in other GI tract-specific tissues including the ileum and the jejunum. This pattern of expression suggests that this gene may be involved in diseases of the digestive tract such as IBS. The highest disease expression is seen in the colon normal and tumor samples. Large upregulation in 1 of 4 colon tumor samples is sufficient to make a claim for colon cancer. Upregulation in 1 of 4 breast tumor samples implicates this gene in breast cancer. Upregulation in 3 of 3 disease heart samples implies an involvement in cardiovascular diseases such as non-obstructive and obstructive DCM and ischemia Upregulation in the HIV-infected PBL cells suggests that this gene may be a host factor in HIV. Upregulation in the W12 cells suggests that this gene may be a host factor in HPV.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1571349- | (sample | (sample | (sample | GOI | rRNA | rRNA | total |
| lipase | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 28.65, 28.66 | 381.49 | 377.43 | 379.46 | 7.24 | 6.91 | 2620.58 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 29.1, 28.56 | 289.29 | 402.29 | 345.79 | 2.71 | 18.45 | 6379.89 |
| Endometrium | 40, 40 | 0 | 0 | 0.00 | 0.73 | 68.21 | 0.00 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 33.37, 34.4 | 21.83 | 11.72 | 16.78 | 2.58 | 19.38 | 325.10 |
| Jejunum | 33.03, 33.89 | 26.82 | 15.94 | 21.38 | 6.60 | 7.58 | 161.97 |
| Kidney | 40, 40 | 0 | 0 | 0.00 | 2.12 | 23.58 | 0.00 |
| Liver | 36.4, 35.75 | 3.49 | 5.18 | 4.34 | 1.50 | 33.33 | 144.50 |
| Fetal Liver Clontech | 33.02 | 27.08 | 82.43 | 54.76 | 10.40 | 4.81 | 263.25 |
| Lung | 40, 40 | 0 | 0.68 | 0.34 | 2.57 | 19.46 | 6.61 |
| Mammary Gland | 38.18, 40 | 1.19 | 0 | 0.60 | 13.00 | 3.85 | 2.29 |
| Clontech | | | | | | | |
| Myometrium | 40, 40 | 0 | 0 | 0.00 | 2.34 | 21.37 | 0.00 |
| Omentum | 40, 40 | 0 | 0 | 0.00 | 3.94 | 12.69 | 0.00 |
| Ovary | 34.85, 34.22 | 8.94 | 13.1 | 11.02 | 4.34 | 11.52 | 126.96 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 38.47, 35.13 | 1 | 7.55 | 4.28 | 5.48 | 9.12 | 39.01 |
| Placenta Clontech | 40, 40 | 0 | 0 | 0.00 | 5.26 | 9.51 | 0.00 |
| Prostate | 40, 40 | 0 | 0 | 0.00 | 3.00 | 16.67 | 0.00 |
| Rectum | 30.07, 30.1 | 161.52 | 157.91 | 159.72 | 1.23 | 40.65 | 6492.48 |
| Salivary Gland Clontech | 32.8, 32.31 | 30.88 | 41.68 | 36.28 | 7.31 | 6.84 | 248.15 |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 40, 40 | 0 | 0 | 0.00 | 4.92 | 10.16 | 0.00 |
| Stomach | 40, 40 | 0 | 0 | 0.00 | 2.73 | 18.32 | 0.00 |
| Testis Clontech | 40, 35.34 | 0 | 6.65 | 3.33 | 0.57 | 87.87 | 292.18 |
| Thymus Clontech | 32.58, 33.71 | 35.33 | 17.82 | 26.58 | 9.89 | 5.06 | 134.35 |
| Thyroid | 40, 35.83 | 0 | 4.93 | 2.47 | 2.77 | 18.05 | 44.49 |
| Trachea Clontech | 37.09, 40 | 2.3 | 0 | 1.15 | 9.71 | 5.15 | 5.92 |
| Urinary Bladder | 40, 40 | 0 | 0 | 0.00 | 5.47 | 9.14 | 0.00 |
| Uterus | 34.81, 34.22 | 9.16 | 13.06 | 11.11 | 5.34 | 9.36 | 104.03 |
| genomic | 26.45 | 1442.6 | | | | | |
| b-actin | 27.74 | 660.76 | | | | | |
| 1.00E+05 | 19.65 | 100000 | | | | | |
| 1.00E+05 | 19.7 | 100000 | | | | | |
| 1.00E+04 | 23.16 | 10000 | | | | | |
| 1.00E+04 | 23.05 | 10000 | | | | | |
| 1.00E+03 | 26.21 | 1000 | | | | | |
| 1.00E+03 | 26.74 | 1000 | | | | | |
| 1.00E+02 | 30.93 | 100 | | | | | |
| 1.00E+02 | 32.11 | 100 | | | | | |
| 1.00E+01 | 33.74 | 10 | | | | | |
| 1.00E+01 | 35.25 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1571349- | (GSK | | GOI | total | | Disease |
| lipase | identifier) | Ct | copies | RNA | Sample | Population |
| colon normal GW98-167 | 21941 | 21.81 | 18527.55 | 37055.10 | colon normal | |
| colon tumor GW98-166 | 21940 | 24.48 | 3977.68 | 7955.36 | colon tumor | −4.66 |
| colon normal GW98-178 | 22080 | 30.22 | 144.61 | 289.22 | colon normal | |
| colon tumor GW98-177 | 22060 | 25.35 | 2406.82 | 4813.64 | colon tumor | 16.64 |
| colon normal GW98-561 | 23514 | 22.94 | 9655.42 | 19310.84 | colon normal | |
| colon tumor GW98-560 | 23513 | 22.94 | 9655.62 | 19311.24 | colon tumor | 1.00 |
| colon normal GW98-894 | 24691 | 21.03 | 29052.3 | 58104.60 | colon normal | |
| colon tumor GW98-893 | 24690 | 23.12 | 8692.86 | 17385.72 | colon tumor | −3.34 |
| lung normal GW98-3 | 20742 | 32.33 | 43.02 | 86.04 | lung normal | |
| lung tumor GW98-2 | 20741 | 40 | 0 | 0.00 | lung tumor | −86.04 |
| lung normal GW97-179 | 20677 | 29.92 | 172.76 | 345.52 | lung normal | |
| lung tumor GW97-178 | 20676 | 31.38 | 74.23 | 148.46 | lung tumor | −2.33 |
| lung normal GW98-165 | 21922 | 31.1 | 87.26 | 174.52 | lung normal | |
| lung tumor GW98-164 | 21921 | 31.13 | 85.73 | 171.46 | lung tumor | −1.02 |
| lung normal GW98-282 | 22584 | 33.29 | 24.7 | 49.40 | lung normal | |
| lung tumor GW98-281 | 22583 | 33.61 | 20.52 | 41.04 | lung tumor | −1.20 |
| breast normal GW00- | 28750 | 33.03 | 28.62 | 28.62 | breast normal | |
| 392 | | | | | | |
| breast tumor GW00-391 | 28746 | 32.43 | 40.5 | 81.00 | breast tumor | 2.83 |
| breast normal GW00- | 28798 | 34.93 | 9.6 | 9.60 | breast normal | |
| 413 | | | | | | |
| breast tumor GW00-412 | 28797 | 33.16 | 26.53 | 53.06 | breast tumor | 5.53 |
| breast normal GW00- | 27592-95 | 34.31 | 13.71 | 13.71 | breast normal | |
| 235:238 | | | | | | |
| breast tumor GWOO- | 27588-91 | 32.53 | 38.16 | 38.16 | breast tumor | 2.78 |
| 231:234 | | | | | | |
| breast normal GW98- | 23656 | 32.24 | 45.24 | 90.48 | breast normal | |
| 621 | | | | | | |
| breast tumor GW98-620 | 23655 | 31.55 | 67.35 | 134.70 | breast tumor | 1.49 |
| brain normal BB99-542 | 25507 | 31.2 | 82.43 | 164.86 | brain normal | |
| brain normal BB99-406 | 25509 | 30.13 | 152.5 | 305.00 | brain normal | |
| brain normal BB99-904 | 25546 | 33.42 | 22.88 | 45.76 | brain normal | |
| brain stage 5 ALZ | 25502 | 31.76 | 59.61 | 119.22 | brain stage 5 ALZ | −1.44 |
| BB99-874 | | | | | | |
| brain stage 5 ALZ | 25503 | 30.05 | 159.48 | 318.96 | brain stage 5 ALZ | 1.86 |
| BB99-887 | | | | | | |
| brain stage 5 ALZ | 25504 | 30.83 | 102.18 | 204.36 | brain stage 5 ALZ | 1.19 |
| BB99-862 | | | | | | |
| brain stage 5 ALZ | 25542 | 30.06 | 159.13 | 318.26 | brain stage 5 ALZ | 1.85 |
| BB99-927 | | | | | | |
| CT lung | normal | 34.2 | 14.61 | 29.22 | CT lung Nml | |
| lung 26 | normal | 30.25 | 142.31 | | lung 26 Nml | |
| lung 27 | normal | 40 | 0 | 0.00 | lung 27 Nml | |
| lung 24 | COPD | 40 | 0 | 0.00 | lung 24 COPD | −9.74 |
| lung 28 | COPD | 40 | 0 | 0.00 | lung 28 COPD | −9.74 |
| lung 23 | COPD | 40 | 0 | 0.00 | lung 23 COPD | −9.74 |
| lung 25 | normal | 40 | 0 | 0.00 | lung 25 Nml | |
| asthmatic lung | 29321 | 36.18 | 4.67 | 4.67 | asthmatic lung | −2.09 |
| ODO3112 | | | | | | |
| asthmatic lung | 29323 | 34.06 | 15.81 | 31.62 | asthmatic lung | 3.25 |
| ODO3433 | | | | | | |
| asthmatic lung | 29322 | 32.15 | 47.6 | 95.20 | asthmatic lung | 9.77 |
| ODO3397 | | | | | | |
| asthmatic lung | 29325 | 35.92 | 5.42 | 10.84 | asthmatic lung | 1.11 |
| ODO4928 | | | | | | |
| endo cells | control | 40 | 0 | 0.00 | endo cells | |
| endo VEGF | | 40 | 0 | 0.00 | endo VEGF | 0.00 |
| endo bFGF | | 40 | 0 | 0.00 | endo bFGF | 0.00 |
| heart Clontech | normal | 40 | 0 | 0.00 | heart | |
| heart (T-1) ischemic | 29417 | 32.63 | 36.17 | 72.34 | heart (T-1) ischemic | 72.34 |
| heart (T-14) non- | 29422 | 32.06 | 50.14 | 100.28 | heart (T-14) non- | 100.28 |
| obstructive DCM | | | | | obstructive DCM | |
| heart (T-3399) DCM | 29426 | 33.12 | 27.25 | 54.50 | heart (T-3399) DCM | 54.50 |
| adenoid GW99-269 | 26162 | 34.14 | 15.07 | 30.14 | adenoid | |
| tonsil GW98-280 | 22582 | 32.93 | 30.43 | 60.86 | tonsil | |
| T cells PC00314 | 28453 | 34.46 | 12.57 | 25.14 | T cells | |
| PBMNC | | 40 | 0 | 0.00 | PBMNC | |
| monocyte | | 40 | 0 | 0.00 | monocyte | |
| B cells PC00665 | 28455 | 32.13 | 48.2 | 96.40 | B cells | |
| dendritic cells | 28441 | 40 | 0 | 0.00 | dendritic cells | |
| neutrophils | 28440 | 36.83 | 3.21 | 3.21 | neutrophils | |
| eosinophils | 28446 | 40 | 0 | 0.00 | eosinophils | |
| BM unstim | | 40 | 0 | 0.00 | BM unstim | |
| BM stim | treated | 40 | 0 | 0.00 | BM stim | 0.00 |
| osteo dif | treated | 40 | 0 | 0.00 | osteo dif | 0.00 |
| osteo undif | | 40 | 0 | 0.00 | osteo undif | |
| chondrocytes | | 31.82 | 57.77 | 144.43 | chondrocytes | |
| OA Synovium IP12/01 | 29462 | 31.1 | 87.17 | 87.17 | OA Synovium | |
| OA Synovium NP10/01 | 29461 | 31.99 | 52.32 | 104.64 | OA Synovium | |
| OA Synovium NP57/00 | 28464 | 30.73 | 107.79 | 215.58 | OA Synovium | |
| RA Synovium | 28466 | 32.67 | 35.27 | 70.54 | RA Synovium | |
| NP03/01 | | | | | | |
| RA Synovium | 28467 | 30.36 | 134.08 | 268.16 | RA Synovium | |
| NP71/00 | | | | | | |
| RA Synovium | 28475 | 32.2 | 46.39 | 92.78 | RA Synovium | |
| NP45/00 | | | | | | |
| OA bone (biobank) | 29217 | 34.27 | 14.02 | 14.02 | OA bone (biobank) | |
| OA bone Sample 1 | J. Emory | 32.28 | 44.28 | 88.56 | OA bone | |
| OA bone Sample 2 | J. Emory | 32.12 | 48.37 | 96.74 | OA bone | |
| Cartilage (pool) | Normal | 29.16 | 267.1 | 534.20 | Nml Cartilage (pool) | |
| Cartilage (pool) | OA | 32.62 | 36.38 | 72.76 | OA Cartilage (pool) | −7.34 |
| PBL unifected | 28441 | 40 | 0 | 0.00 | PBL unifected | |
| PBL HIV IIIB | 28442 | 34.18 | 14.74 | 29.48 | PBL HIV IIIB | 29.48 |
| MRC5 uninfected | 29158 | 32.13 | 48.26 | 96.52 | MRC5 uninfected | |
| (100%) | | | | | (100%) | |
| MRC5 HSV strain F | 29178 | 31.75 | 60.03 | 120.06 | MRC5 HSV strain F | 1.24 |
| W12 cells | 29179 | 32.75 | 33.72 | 67.44 | W12 cells | 67.44 |
| Keratinocytes | 29180 | 40 | 0 | 0.00 | Keratinocytes | |
| B-actin control | | 27.39 | 743.52 | | | |
| genomic | | 26.45 | 1273.58 | | | |
| 1.00E+05 | | 19.22 | 100000 | | | |
| 1.00E+05 | | 19.32 | 100000 | | | |
| 1.00E+04 | | 22.57 | 10000 | | | |
| 1.00E+04 | | 22.44 | 10000 | | | |
| 1.00E+03 | | 26.39 | 1000 | | | |
| 1.00E+03 | | 26.17 | 1000 | | | |
| 1.00E+02 | | 31.23 | 100 | | | |
| 1.00E+02 | | 32.05 | 100 | | | |
| 1.00E+01 | | 33.62 | 10 | | | |
| 1.00E+01 | | 35.71 | 10 | | | |
| 1.00E+00 | | 40 | 0 | | | |
| 1.00E+00 | | 40 | 1 | | | |
| NTC | | 40 | 0 | | | |

*lung 26 Normal has been omitted due to multiple amplification failures from that sample

Gene Name sbg1571349lipase

| | Fold Change in Disease |
|---|---|
| | Population Relative to |
| Disease tissues | Normal |
| colon tumor | −4.66 |
| colon tumor | 16.64 |
| colon tumor | 1.00 |
| colon tumor | −3.34 |
| lung tumor | −86.04 |
| lung tumor | −2.33 |
| lung tumor | −1.02 |
| lung tumor | −1.20 |
| breast tumor | 2.83 |
| breast tumor | 5.53 |
| breast tumor | 2.78 |
| breast tumor | 1.49 |
| brain stage 5 ALZ | −1.44 |
| brain stage 5 ALZ | 1.86 |
| brain stage 5 ALZ | 1.19 |
| brain stage 5 ALZ | 1.85 |
| lung 24 COPD | −9.74 |
| lung 28 COPD | −9.74 |
| lung 23 COPD | −9.74 |
| asthmatic lung | −2.09 |
| asthmatic lung | 3.25 |
| asthmatic lung | 9.77 |
| asthmatic lung | 1.11 |
| endo VEGF | 0.00 |
| endo bFGF | 0.00 |
| heart (T-1) ischemic | 72.34 |
| heart (T-14) non-obstructive DCM | 100.28 |
| heart (T-3399) DCM | 54.50 |
| BM stim | 0.00 |
| osteo dif | 0.00 |
| OA Cartilage (pool) | −7.34 |
| PBL HIV IIIB | 29.48 |
| MRC5 HSV strain F | 1.24 |
| W12 cells | 67.44 |

Gene Name sbg1565539aCONNEXIN

Failed

Gene Name sbg1565539bCONNEXIN

Highly specific overall expression. The highest normal expression is seen primarily in tissues representing the female reproductive system including the endometrium, ovary, placenta, and uterus as well as tissues representing the GI tract such as the colon, ileum, jejunum, omentum, and stomach. Additional high expression is seen in the whole brain and the liver. Highest disease expression is specific to the Alzheimer's brain samples. Upregulation in 2 of 3 disease heart samples implies an involvement in cardiovascular diseases such as obstructive DCM and ischemia. Upregulated in the differentiated osteoblasts. Downregulation in the HIV-infected PBLs and the HSV-infected MRC5 cells suggests that this gene may be a host factor in HIV and HSV. High expression in the RA and OA synovium samples, the OA bone samples, and the chondrocytes with corroborating high expression in the T cells, B cells, and neutrophils implicates this gene in osteoarthritis and rheumatoid arthritis. Upregulation in the W12 cells suggests that this gene may be a host factor in HPV.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1565539b- | (sample | (sample | (sample | GOI | rRNA | rRNA | total |
| CONNEXIN | 1 and 2) | 1) | 2) | Copies | (ng) | (ng) | RNA |
| Subcutaneous Adipocytes | 36.28, 40 | 5.2 | 0 | 2.60 | 3.06 | 16.34 | 42.48 |
| Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 28.42, 28.6 | 530.94 | 479.68 | 505.31 | 7.24 | 6.91 | 3489.71 |
| Fetal Brain Clontech | 40, 40 | 0 | 0 | 0.00 | 0.48 | 103.95 | 0.00 |
| Cerebellum Clontech | 40, 39.91 | 0 | 4.34 | 2.17 | 2.17 | 23.04 | 50.00 |
| Cervix | 40, 40 | 0 | 0 | 0.00 | 2.42 | 20.66 | 0.00 |
| Colon | 40, 37.11 | 0 | 3.19 | 1.60 | 2.71 | 18.45 | 29.43 |
| Endometrium | 40, 36.59 | 0 | 4.34 | 2.17 | 0.73 | 68.21 | 148.02 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 35.95 | 0 | 6.3 | 3.15 | 2.58 | 19.38 | 61.05 |
| Jejunum | 35.01, 35.1 | 11 | 10.42 | 10.71 | 6.60 | 7.58 | 81.14 |
| Kidney | 40, 40 | 0 | 0 | 0.00 | 2.12 | 23.58 | 0.00 |
| Liver | 35.14, 35.33 | 10.15 | 9.1 | 9.63 | 1.50 | 33.33 | 320.83 |
| Fetal Liver Clontech | 35.29, 34.74 | 9.33 | 12.9 | 11.12 | 10.40 | 4.81 | 53.44 |
| Lung | 40, 40 | 0 | 0 | 0.00 | 2.57 | 19.46 | 0.00 |
| Mammary Gland | 40, 36.16 | 0 | 5.58 | 2.79 | 13.00 | 3.85 | 10.73 |
| Clontech | | | | | | | |
| Myometrium | 40, 40 | 0 | 0 | 0.00 | 2.34 | 21.37 | 0.00 |
| Omentum | 35.17, 33.81 | 9.98 | 22.2 | 16.09 | 3.94 | 12.69 | 204.19 |
| Ovary | 40, 34.09 | 0 | 18.92 | 9.46 | 4.34 | 11.52 | 108.99 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 35.74, 35.13 | 7.14 | 10.21 | 8.68 | 5.48 | 9.12 | 79.15 |
| Placenta Clontech | 34.95, 35.76 | 11.39 | 7.07 | 9.23 | 5.26 | 9.51 | 87.74 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland Clontech | 40, 36.02 | 0 | 6.04 | 3.02 | 7.31 | 6.84 | 20.66 |
| Skeletal Muscle Clontech | 40, 40 | 0 | 0 | 0.00 | 1.26 | 39.68 | 0.00 |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 35.14, 40 | 10.2 | 0 | 5.10 | 4.92 | 10.16 | 51.83 |
| Stomach | 36.06, 35.41 | 5.92 | 8.67 | 7.30 | 2.73 | 18.32 | 133.61 |
| Testis Clontech | 40, 40 | 0 | 0 | 0.00 | 0.57 | 87.87 | 0.00 |
| Thymus Clontech | 35.49, 40 | 8.25 | 0 | 4.13 | 9.89 | 5.06 | 20.85 |
| Thyroid | 40, 40 | 0 | 0 | 0.00 | 2.77 | 18.05 | 0.00 |
| Trachea Clontech | 40, 40 | 0 | 0 | 0.00 | 9.71 | 5.15 | 0.00 |
| Urinary Bladder | 40, 40 | 0 | 0 | 0.00 | 5.47 | 9.14 | 0.00 |
| Uterus | 35.46, 34.1 | 8.44 | 18.73 | 13.59 | 5.34 | 9.36 | 127.20 |
| genomic | 27.32 | 1018.5 | | | | | |
| b-actin | 28.07 | 654.85 | | | | | |
| 1.00E+05 | 19.58 | 100000 | | | | | |
| 1.00E+05 | 19.75 | 100000 | | | | | |
| 1.00E+04 | 23.28 | 10000 | | | | | |
| 1.00E+04 | 23.36 | 10000 | | | | | |
| 1.00E+03 | 27.08 | 1000 | | | | | |
| 1.00E+03 | 27.26 | 1000 | | | | | |
| 1.00E+02 | 31.3 | 100 | | | | | |
| 1.00E+02 | 31.53 | 100 | | | | | |
| 1.00E+01 | 40 | 10 | | | | | |
| 1.00E+01 | 40 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1565539b- | (GSK | | GOI | total | | Disease |
| CONNEXIN | identifier) | Ct | copies | RNA | Sample | Population |
| colon normal GW98-167 | 21941 | 34.17 | 36.97 | 73.94 | colon normal | |
| colon tumor GW98-166 | 21940 | 34.12 | 38.19 | 76.38 | colon tumor | 1.03 |
| colon normal GW98-178 | 22080 | 36.65 | 9.19 | 18.38 | colon normal | |
| colon tumor GW98-177 | 22060 | 34.02 | 40.31 | 80.62 | colon tumor | 4.39 |
| colon normal GW98-561 | 23514 | 36.43 | 10.4 | 20.80 | colon normal | |
| colon tumor GW98-560 | 23513 | 35.4 | 18.5 | 37.00 | colon tumor | 1.78 |
| colon normal GW98-894 | 24691 | 33.79 | 46 | 92.00 | colon normal | |
| colon tumor GW98-893 | 24690 | 35.1 | 21.92 | 43.84 | colon tumor | −2.10 |
| lung normal GW98-3 | 20742 | 33.16 | 65.36 | 130.72 | lung normal | |
| lung tumor GW98-2 | 20741 | 40 | 0 | 0.00 | lung tumor | −130.72 |
| lung normal GW97-179 | 20677 | 33.44 | 55.88 | 111.76 | lung normal | |
| lung tumor GW97-178 | 20676 | 33.44 | 55.89 | 111.78 | lung tumor | 1.00 |
| lung normal GW98-165 | 21922 | 32.96 | 73.02 | 146.04 | lung normal | |
| lung tumor GW98-164 | 21921 | 32.32 | 104.66 | 209.32 | lung tumor | 1.43 |
| lung normal GW98-282 | 22584 | 40 | 0 | 0.00 | lung normal | |
| lung tumor GW98-281 | 22583 | 34.38 | 32.96 | 65.92 | lung tumor | 65.92 |
| breast normal GW00-392 | 28750 | 33.59 | 51.32 | 51.32 | breast normal | |
| breast tumor GW00-391 | 28746 | 33.66 | 49.38 | 98.76 | breast tumor | 1.92 |
| breast normal GW00-413 | 28798 | 33.6 | 51.05 | 51.05 | breast normal | |
| breast tumor GW00-412 | 28797 | 34.67 | 28.04 | 56.08 | breast tumor | 1.10 |
| breast normal GW00- | 27592-95 | 33.2 | 63.96 | 63.96 | breast normal | |
| 235:238 | | | | | | |
| breast tumor GW00- | 27588-91 | 33.09 | 68.07 | 68.07 | breast tumor | 1.06 |
| 231:234 | | | | | | |
| breast normal GW98-621 | 23656 | 33.23 | 62.78 | 125.56 | breast normal | |
| breast tumor GW98-620 | 23655 | 33.89 | 43.44 | 86.88 | breast tumor | −1.45 |
| brain normal BB99-542 | 25507 | 29.44 | 528.77 | 1057.54 | brain normal | |
| brain normal BB99-406 | 25509 | 32.03 | 123.74 | 247.48 | brain normal | |
| brain normal BB99-904 | 25546 | 29 | 679.06 | 1358.12 | brain normal | |
| brain stage 5 ALZ BB99- | 25502 | 32.89 | 76.09 | 152.18 | brain stage 5 ALZ | −5.83 |
| 874 | | | | | | |
| brain stage 5 ALZ BB99- | 25503 | 30.24 | 337.73 | 675.46 | brain stage 5 ALZ | −1.31 |
| 887 | | | | | | |
| brain stage 5 ALZ BB99- | 25504 | 30.43 | 303.67 | 607.34 | brain stage 5 ALZ | −1.46 |
| 862 | | | | | | |
| brain stage 5 ALZ BB99- | 25542 | 29.62 | 478.76 | 957.52 | brain stage 5 ALZ | 1.08 |
| 927 | | | | | | |
| CT lung | normal | 40 | 0 | 0.00 | CT lung Nml | |
| lung 26 | normal | 40 | 1015.6 | | lung 26 Nml | |
| lung 27 | normal | 36.73 | 8.79 | 8.79 | lung 27 Nml | |
| lung 24 | COPD | 40 | 0 | 0.00 | lung 24 COPD | −9.84 |
| lung 28 | COPD | 40 | 0 | 0.00 | lung 28 COPD | −9.84 |
| lung 23 | COPD | 35.64 | 16.25 | 16.25 | lung 23 COPD | 1.65 |
| lung 25 | normal | 35.2 | 20.72 | 20.72 | lung 25 Nml | |
| asthmatic lung ODO3112 | 29321 | 35.75 | 15.26 | 15.26 | asthmatic lung | 1.55 |
| asthmatic lung ODO3433 | 29323 | 34.63 | 28.66 | 57.32 | asthmatic lung | 5.83 |
| asthmatic lung ODO3397 | 29322 | 36.61 | 9.41 | 18.82 | asthmatic lung | 1.91 |
| asthmatic lung ODO4928 | 29325 | 34.2 | 36.36 | 72.72 | asthmatic lung | 7.39 |
| endo cells | control | 40 | 0 | 0.00 | endo cells | |
| endo VEGF | | 40 | 0 | 0.00 | endo VEGF | 0.00 |
| endo bFGF | | 40 | 0 | 0.00 | endo bFGF | 0.00 |
| heart Clontech | normal | 40 | 0 | 0.00 | heart | |
| heart (T-1) ischemic | 29417 | 35.4 | 18.58 | 37.16 | heart (T-1) | 37.16 |
| | | | | | ischemic | |
| heart (T-14) non- | 29422 | 40 | 0 | 0.00 | heart (T-14) non- | 0.00 |
| obstructive DCM | | | | | obstructive DCM | |
| heart (T-3399) DCM | 29426 | 35.3 | 19.59 | 39.18 | heart (T-3399) DCM | 39.18 |
| adenoid GW99-269 | 26162 | 40 | 0 | 0.00 | adenoid | |
| tonsil GW98-280 | 22582 | 40 | 0 | 0.00 | tonsil | |
| T cells PC00314 | 28453 | 31.96 | 128.72 | 257.44 | T cells | |
| PBMNC | | 40 | 0 | 0.00 | PBMNC | |
| monocyte | | 36.29 | 11.27 | 22.54 | monocyte | |
| B cells PC00665 | 28455 | 33.69 | 48.6 | 97.20 | B cells | |
| dendritic cells | 28441 | 40 | 0 | 0.00 | dendritic cells | |
| neutrophils | 28440 | 32.43 | 98.82 | 98.82 | neutrophils | |
| eosinophils | 28446 | 34.77 | 26.38 | 52.76 | eosinophils | |
| BM unstim | | 40 | 0 | 0.00 | BM unstim | |
| BM stim | treated | 40 | 0 | 0.00 | BM stim | 0.00 |
| osteo dif | treated | 35.04 | 22.76 | 22.76 | osteo dif | 22.76 |
| osteo undif | | 40 | 0 | 0.00 | osteo undif | |
| chondrocytes | | 32.27 | 107.58 | 268.95 | chondrocytes | |
| OA Synovium IP12/01 | 29462 | 32.64 | 87.38 | 87.38 | OA Synovium | |
| OA Synovium NP10/01 | 29461 | 35.16 | 21.21 | 42.42 | OA Synovium | |
| OA Synovium NP57/00 | 28464 | 32.72 | 83.67 | 167.34 | OA Synovium | |
| RA Synovium NP03/01 | 28466 | 33.11 | 67.32 | 134.64 | RA Synovium | |
| RA Synovium | 28467 | 34.14 | 37.67 | 75.34 | RA Synovium | |
| NP71/00 | | | | | | |
| RA Synovium | 28475 | 33.02 | 70.9 | 141.80 | RA Synovium | |
| NP45/00 | | | | | | |
| OA bone (biobank) | 29217 | 34.82 | 25.76 | 25.76 | OA bone (biobank) | |
| OA bone Sample 1 | J. Emory | 31.76 | 143.77 | 287.54 | OA bone | |
| OA bone Sample 2 | J. Emory | 33.25 | 62.21 | 124.42 | OA bone | |
| Cartilage (pool) | Normal | 32.24 | 109.9 | 219.80 | Nml Cartilage (pool) | |
| Cartilage (pool) | OA | 33.41 | 56.7 | 113.40 | OA Cartilage (pool) | −1.94 |
| PBL unifected | 28441 | 33.37 | 58.1 | 116.20 | PBL unifected | |
| PBL HIV IIIB | 28442 | 34.85 | 25.34 | 50.68 | PBL HIV IIIB | −2.29 |
| MRC5 uninfected | 29158 | 32.53 | 93.06 | 186.12 | MRC5 uninfected | |
| (100%) | | | | | (100%) | |
| MRC5 HSV strain F | 29178 | 34.4 | 32.5 | 65.00 | MRC5 HSV strain F | −2.86 |
| W12 cells | 29179 | 40 | 1.27 | 2.54 | W12 cells | 2.54 |
| Keratinocytes | 29180 | 40 | 0 | 0.00 | Keratinocytes | |
| B-actin control | | 27.94 | 1228.38 | | | |
| genomic | | 27.61 | 1485.33 | | | |
| 1.00E+05 | | 20.29 | 100000 | | | |
| 1.00E+05 | | 20.32 | 100000 | | | |
| 1.00E+04 | | 24.09 | 10000 | | | |
| 1.00E+04 | | 24.12 | 10000 | | | |
| 1.00E+03 | | 28.12 | 1000 | | | |
| 1.00E+03 | | 28.02 | 1000 | | | |
| 1.00E+02 | | 32.15 | 100 | | | |
| 1.00E+02 | | 32.83 | 100 | | | |
| 1.00E+01 | | 37.6 | 10 | | | |
| 1.00E+01 | | 35.56 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1565539bCONNEXIN

| | Fold Change in Disease |
|---|---|
| | Population Relative to |
| Disease tissues | Normal |
| colon tumor | 1.03 |
| colon tumor | 4.39 |
| colon tumor | 1.78 |
| colon tumor | −2.10 |
| lung tumor | −130.72 |
| lung tumor | 1.00 |
| lung tumor | 1.43 |
| lung tumor | 65.92 |
| breast tumor | 1.92 |
| breast tumor | 1.10 |
| breast tumor | 1.06 |
| breast tumor | −1.45 |
| brain stage 5 ALZ | −5.83 |
| brain stage 5 ALZ | −1.31 |
| brain stage 5 ALZ | −1.46 |
| brain stage 5 ALZ | 1.08 |
| lung 24 COPD | −9.84 |
| lung 28 COPD | −9.84 |
| lung 23 COPD | 1.65 |
| asthmatic lung | 1.55 |
| asthmatic lung | 5.83 |
| asthmatic lung | 1.91 |
| asthmatic lung | 7.39 |
| endo VEGF | 0.00 |
| endo bFGF | 0.00 |
| heart (T-1) ischemic | 37.16 |
| heart (T-14) non-obstructive DCM | 0.00 |
| heart (T-3399) DCM | 39.18 |
| BM stim | 0.00 |
| osteo dif | 22.76 |
| OA Cartilage (pool) | −1.94 |
| PBL HIV IIIB | −2.29 |
| MRC5 HSV strain F | −2.86 |
| W12 cells | 2.54 |

Gene Name sbg1563294acadherin

Moderate to low overall expression. The highest normal expression is seen in the whole brain, prostate, thymus, and trachea. The highest disease expression is seen in some of the colon and lung normal and tumor samples as well as in 2 of the asthmatic lung samples. Upregulation in 1 of 4 colon tumor samples and 1 of 4 lung tumor samples implicates this gene in cancers of the colon and lung. Upregulation in 3 of 4 asthmatic lung samples coupled with immune cell expression in indicates a possible involvement in asthma. Upregulation in 3 of 3 disease heart samples implies an involvement in cardiovascular diseases such as non-obstructive and obstructive DCM and ischemia. This gene appears to show the same expression patterns in the disease samples as 1563294b. Based on this, it is likely that these two different primer sets are amplifying the same gene.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1563294a- | (sample | (sample | (sample | GOI | rRNA | rRNA | total |
| cadherin | 1 and 2) | 1) | 2) | Copies | (ng) | (ng) | RNA |
| Subcutaneous | 35.18, 34.05 | 10.16 | 20.47 | 15.32 | 3.06 | 16.34 | 250.25 |
| Adipocytes Zenbio | | | | | | | |
| Subcutaneous | 40, 37.07 | 0 | 3.12 | 1.56 | 0.96 | 52.36 | 81.68 |
| Adipose Zenbio | | | | | | | |
| Adrenal Gland | 36.83, 37.11 | 3.63 | 3.04 | 3.34 | 0.61 | 81.97 | 273.36 |
| Clontech | | | | | | | |
| Whole Brain Clontech | 28.05, 27.84 | 855.18 | 975.02 | 915.10 | 7.24 | 6.91 | 6319.75 |
| Fetal Brain Clontech | 40, 40 | 0 | 0 | 0.00 | 0.48 | 103.95 | 0.00 |
| Cerebellum Clontech | 36.55, 36.15 | 4.33 | 5.53 | 4.93 | 2.17 | 23.04 | 113.59 |
| Cervix | 40, 37.37 | 0 | 2.59 | 1.30 | 2.42 | 20.66 | 26.76 |
| Colon | 36.27, 35.14 | 5.13 | 10.4 | 7.77 | 2.71 | 18.45 | 143.27 |
| Endometrium | 40, 40 | 0 | 0 | 0.00 | 0.73 | 68.21 | 0.00 |
| Esophagus | 36.74, 40 | 3.84 | 0 | 1.92 | 1.37 | 36.50 | 70.07 |
| Heart Clontech | 40, 37.03 | 0 | 3.2 | 1.60 | 1.32 | 37.88 | 60.61 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 34.97, 33.38 | 11.53 | 31.09 | 21.31 | 6.60 | 7.58 | 161.44 |
| Kidney | 40, 35.03 | 0 | 11.1 | 5.55 | 2.12 | 23.58 | 130.90 |
| Liver | 40, 36.73 | 0 | 3.86 | 1.93 | 1.50 | 33.33 | 64.33 |
| Fetal Liver Clontech | 32.3, 32.52 | 60.85 | 53.09 | 56.97 | 10.40 | 4.81 | 273.89 |
| Lung | 33.6, 34.06 | 27.01 | 20.39 | 23.70 | 2.57 | 19.46 | 461.09 |
| Mammary Gland | 32.92, 32.24 | 41.47 | 63.25 | 52.36 | 13.00 | 3.85 | 201.38 |
| Clontech | | | | | | | |
| Myometrium | 40, 39.48 | 0 | 0.7 | 0.35 | 2.34 | 21.37 | 7.48 |
| Omentum | 34.65, 34.7 | 14.12 | 13.68 | 13.90 | 3.94 | 12.69 | 176.40 |
| Ovary | 35.24, 37.08 | 9.75 | 3.11 | 6.43 | 4.34 | 11.52 | 74.08 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 35.56, 36.73 | 7.98 | 3.87 | 5.93 | 5.48 | 9.12 | 54.06 |
| Placenta Clontech | 34.66, 36.1 | 13.99 | 5.72 | 9.86 | 5.26 | 9.51 | 93.68 |
| Prostate | 32.65, 33.31 | 48.83 | 32.38 | 40.61 | 3.00 | 16.67 | 676.75 |
| Rectum | 36.01, 40 | 6.03 | 0 | 3.02 | 1.23 | 40.65 | 122.56 |
| Salivary Gland | 36.61, 35.77 | 4.15 | 7 | 5.58 | 7.31 | 6.84 | 38.13 |
| Clontech | | | | | | | |
| Skeletal Muscle | 40, 37.84 | 0 | 1.93 | 0.97 | 1.26 | 39.68 | 38.29 |
| Clontech | | | | | | | |
| Skin | 40, 38.04 | 0 | 1.71 | 0.86 | 1.21 | 41.32 | 35.33 |
| Small Intestine | 36.86, 40 | 3.56 | 0 | 1.78 | 0.98 | 51.07 | 90.91 |
| Clontech | | | | | | | |
| Spleen | 33.78, 34.67 | 24.18 | 13.89 | 19.04 | 4.92 | 10.16 | 193.45 |
| Stomach | 40, 35.96 | 0.37 | 6.22 | 3.30 | 2.73 | 18.32 | 60.35 |
| Testis Clontech | 40, 36.66 | 0 | 4.04 | 2.02 | 0.57 | 87.87 | 177.50 |
| Thymus Clontech | 30, 29.53 | 254.19 | 341.24 | 297.72 | 9.89 | 5.06 | 1505.13 |
| Thyroid | 36.28, 35.31 | 5.1 | 9.34 | 7.22 | 2.77 | 18.05 | 130.32 |
| Trachea Clontech | 31.45, 30.55 | 103.03 | 180.44 | 141.74 | 9.71 | 5.15 | 729.84 |
| Urinary Bladder | 35.12, 34.3 | 10.54 | 17.5 | 14.02 | 5.47 | 9.14 | 128.15 |
| Uterus | 33.99, 34.37 | 21.21 | 16.81 | 19.01 | 5.34 | 9.36 | 178.00 |
| genomic | 27.38 | 1297.04 | | | | | |
| b-actin | 28.45 | 666.41 | | | | | |
| 1.00E+05 | 20.54 | 100000 | | | | | |
| 1.00E+05 | 20.49 | 100000 | | | | | |
| 1.00E+04 | 23.66 | 10000 | | | | | |
| 1.00E+04 | 24.02 | 10000 | | | | | |
| 1.00E+03 | 27.83 | 1000 | | | | | |
| 1.00E+03 | 27.45 | 1000 | | | | | |
| 1.00E+02 | 32.08 | 100 | | | | | |
| 1.00E+02 | 32.23 | 100 | | | | | |
| 1.00E+01 | 33.15 | 10 | | | | | |
| 1.00E+01 | 36.57 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1563294a- | (GSK | | GOI | total | | Disease |
| cadherin | identifier) | Ct | copies | RNA | Sample | Population |
| colon normal GW98-167 | 21941 | 30.45 | 272.65 | 545.30 | colon normal | |
| colon tumor GW98-166 | 21940 | 29.54 | 466.79 | 933.58 | colon tumor | 1.71 |
| colon normal GW98-178 | 22080 | 32.11 | 102.37 | 204.74 | colon normal | |
| colon tumor GW98-177 | 22060 | 30.73 | 232.2 | 464.40 | colon tumor | 2.27 |
| colon normal GW98-561 | 23514 | 31.25 | 170.2 | 340.40 | colon normal | |
| colon tumor GW98-560 | 23513 | 25.38 | 5486.82 | 10973.64 | colon tumor | 32.24 |
| colon normal GW98-894 | 24691 | 31.16 | 180.03 | 360.06 | colon normal | |
| colon tumor GW98-893 | 24690 | 31.44 | 151.77 | 303.54 | colon tumor | −1.19 |
| lung normal GW98-3 | 20742 | 26.23 | 3322.51 | 6645.02 | lung normal | |
| lung tumor GW98-2 | 20741 | 28.13 | 1078.21 | 2156.42 | lung tumor | −3.08 |
| lung normal GW97-179 | 20677 | 28.53 | 847.92 | 1695.84 | lung normal | |
| lung tumor GW97-178 | 20676 | 25.6 | 4821.54 | 9643.08 | lung tumor | 5.69 |
| lung normal GW98-165 | 21922 | 26.55 | 2749.53 | 5499.06 | lung normal | |
| lung tumor GW98-164 | 21921 | 25.19 | 6129.4 | 12258.80 | lung tumor | 2.23 |
| lung normal GW98-282 | 22584 | 26.14 | 3498.08 | 6996.16 | lung normal | |
| lung tumor GW98-281 | 22583 | 28.57 | 832.53 | 1665.06 | lung tumor | −4.20 |
| breast normal GW00-392 | 28750 | 29.87 | 385.61 | 385.61 | breast normal | |
| breast tumor GW00-391 | 28746 | 30.46 | 272.26 | 544.52 | breast tumor | 1.41 |
| breast normal GW00-413 | 28798 | 30.67 | 240.34 | 240.34 | breast normal | |
| breast tumor GW00-412 | 28797 | 30.48 | 268.96 | 537.92 | breast tumor | 2.24 |
| breast normal GW00- | 27592-95 | 30.45 | 273.76 | 273.76 | breast normal | |
| 235:238 | | | | | | |
| breast tumor GW00- | 27588-91 | 30.21 | 315.33 | 315.33 | breast tumor | 1.15 |
| 231:234 | | | | | | |
| breast normal GW98-621 | 23656 | 29.69 | 427.33 | 854.66 | breast normal | |
| breast tumor GW98-620 | 23655 | 30.06 | 345.05 | 690.10 | breast tumor | −1.24 |
| brain normal BB99-542 | 25507 | 27.8 | 1311.06 | 2622.12 | brain normal | |
| brain normal BB99-406 | 25509 | 27.43 | 1626.56 | 3253.12 | brain normal | |
| brain normal BB99-904 | 25546 | 30.13 | 330.31 | 660.62 | brain normal | |
| brain stage 5 | 25502 | 29.06 | 622.62 | 1245.24 | brain stage 5 ALZ | −1.75 |
| ALZ BB99-874 | | | | | | |
| brain stage 5 | 25503 | 27.07 | 2020.76 | 4041.52 | brain stage 5 ALZ | 1.86 |
| ALZ BB99-887 | | | | | | |
| brain stage 5 | 25504 | 28.16 | 1058.41 | 2116.82 | brain stage 5 ALZ | −1.03 |
| ALZ BB99-862 | | | | | | |
| brain stage 5 | 25542 | 28.29 | 978.87 | 1957.74 | brain stage 5 ALZ | −1.11 |
| ALZ BB99-927 | | | | | | |
| CT lung | normal | 28.86 | 699.64 | 1399.28 | CT lung Nml | |
| lung 26 | normal | 27.67 | 1416.44 | | lung 26 Nml | |
| lung 27 | normal | 31.28 | 167.59 | 167.59 | lung 27 Nml | |
| lung 24 | COPD | 31.37 | 158.47 | 158.47 | lung 24 COPD | 4.02 |
| lung 28 | COPD | 31.09 | 187.38 | 187.38 | lung 28 COPD | −3.40 |
| lung 23 | COPD | 29.49 | 482.41 | 482.41 | lung 23 COPD | −1.32 |
| lung 25 | normal | 30.05 | 346.64 | 346.64 | lung 25 Nml | |
| asthmatic lung ODO3112 | 29321 | 28.32 | 965.18 | 965.18 | asthmatic lung | 1.51 |
| asthmatic lung ODO3433 | 29323 | 27.74 | 1354.63 | 2709.26 | asthmatic lung | 4.25 |
| asthmatic lung ODO3397 | 29322 | 28.08 | 1112.71 | 2225.42 | asthmatic lung | 3.49 |
| asthmatic lung ODO4928 | 29325 | 28.13 | 1075.22 | 2150.44 | asthmatic lung | 3.37 |
| endo cells | control | 40 | 0 | 0.00 | endo cells | |
| endo VEGF | | 35.79 | 11.64 | 11.64 | endo VEGF | 11.64 |
| endo bFGF | | 40 | 0 | 0.00 | endo bFGF | 0.00 |
| heart Clontech | normal | 35.89 | 10.93 | 21.86 | heart | |
| heart (T-1) ischemic | 29417 | 31.1 | 186.02 | 372.04 | heart (T-1) | 17.02 |
| | | | | | ischemic | |
| heart (T-14) non- | 29422 | 31.91 | 115.38 | 230.76 | heart (T-14) non- | 10.56 |
| obstructive DCM | | | | | obstructive DCM | |
| heart (T-3399) DCM | 29426 | 30.71 | 234.8 | 469.60 | heart (T-3399) | 21.48 |
| | | | | | DCM | |
| adenoid GW99-269 | 26162 | 27.78 | 1325.3 | 2650.60 | adenoid | |
| tonsil GW98-280 | 22582 | 26.94 | 2181.1 | 4362.20 | tonsil | |
| T cells PC00314 | 28453 | 30.14 | 328.5 | 657.00 | T cells | |
| PBMNC | | 34.06 | 32.41 | 32.41 | PBMNC | |
| monocyte | | 32.1 | 103.22 | 206.44 | monocyte | |
| B cells PC00665 | 28455 | 30.49 | 266.46 | 532.92 | B cells | |
| dendritic cells | 28441 | 29.8 | 400.89 | 801.78 | dendritic cells | |
| neutrophils | 28440 | 28.31 | 966.8 | 966.80 | neutrophils | |
| eosinophils | 28446 | 29.5 | 478.06 | 956.12 | eosinophils | |
| BM unstim | | 31.09 | 187.47 | 187.47 | BM unstim | |
| BM stim | treated | 31.53 | 144.53 | 144.53 | BM stim | −1.30 |
| osteo dif | treated | 36.7 | 6.78 | 6.78 | osteo dif | 1.20 |
| osteo undif | | 37 | 5.67 | 5.67 | osteo undif | |
| chondrocytes | | 33.79 | 37.96 | 94.90 | chondrocytes | |
| OA Synovium IP12/01 | 29462 | 29.29 | 543.87 | 543.87 | OA Synovium | |
| OA Synovium NP10/01 | 29461 | 30.73 | 231.25 | 462.50 | OA Synovium | |
| OA Synovium NP57/00 | 28464 | 30.01 | 354.05 | 708.10 | OA Synovium | |
| RA Synovium | 28466 | 30.19 | 319.47 | 638.94 | RA Synovium | |
| NP03/01 | | | | | | |
| RA Synovium | 28467 | 29.59 | 454.45 | 908.90 | RA Synovium | |
| NP71/00 | | | | | | |
| RA Synovium | 28475 | 30.26 | 306.51 | 613.02 | RA Synovium | |
| NP45/00 | | | | | | |
| OA bone (biobank) | 29217 | 30.44 | 274.99 | 274.99 | OA bone | |
| | | | | | (biobank) | |
| OA bone Sample 1 | J. Emory | 30.58 | 253.73 | 507.46 | OA bone | |
| OA bone Sampl 2 | J. Emory | 29.5 | 478.67 | 957.34 | OA bone | |
| Cartilage (pool) | Normal | 29.84 | 392.34 | 784.68 | Nml Cartilage | |
| | | | | | (pool) | |
| Cartilage (pool) | OA | 32.44 | 84.12 | 168.24 | OA Cartilage | −4.66 |
| | | | | | (pool) | |
| PBL unifected | 28441 | 30.99 | 198.67 | 397.34 | PBL unifected | |
| PBL HIV IIIB | 28442 | 31.5 | 146.74 | 293.48 | PBL HIV IIIB | −1.35 |
| MRC5 uninfected (100%) | 29158 | 34.39 | 26.52 | 53.04 | MRC5 | |
| | | | | | uninfected | |
| | | | | | (100%) | |
| MRC5 HSV strain F | 29178 | 35.37 | 14.92 | 29.84 | MRC5 HSV | −1.78 |
| | | | | | strain F | |
| W12 cells | 29179 | 30.15 | 325.42 | 650.84 | W12 cells | −1.01 |
| Keratinocytes | 29180 | 30.14 | 327.34 | 654.68 | Keratinocytes | |
| B-actin control | | 28.07 | 1118.54 | | | |
| genomic | | 27.26 | 1802.46 | | | |
| 1.00E+05 | | 20.95 | 100000 | | | |
| 1.00E+05 | | 20.95 | 100000 | | | |
| 1.00E+04 | | 24.15 | 10000 | | | |
| 1.00E+04 | | 23.99 | 10000 | | | |
| 1.00E+03 | | 27.62 | 1000 | | | |
| 1.00E+03 | | 27.5 | 1000 | | | |
| 1.00E+02 | | 32.65 | 100 | | | |
| 1.00E+02 | | 32.29 | 100 | | | |
| 1.00E+01 | | 36.41 | 10 | | | |
| 1.00E+01 | | 40 | 0 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1563294acadherin

| | Fold Change in Disease |
|---|---|
| | Population Relative to |
| Disease tissues | Normal |
| colon tumor | 1.71 |
| colon tumor | 2.27 |
| colon tumor | 32.24 |
| colon tumor | −1.19 |
| lung tumor | −3.08 |
| lung tumor | 5.69 |
| lung tumor | 2.23 |
| lung tumor | −4.20 |
| breast tumor | 1.41 |
| breast tumor | 2.24 |
| breast tumor | 1.15 |
| breast tumor | −1.24 |
| brain stage 5 ALZ | −1.75 |
| brain stag 5 ALZ | 1.86 |
| brain stage 5 ALZ | −1.03 |
| brain stage 5 ALZ | −1.11 |
| lung 24 COPD | −4.02 |
| lung 28 COPD | −3.40 |
| lung 23 COPD | −1.32 |
| asthmatic lung | 1.51 |
| asthmatic lung | 4.25 |
| asthmatic lung | 3.49 |
| asthmatic lung | 3.37 |
| endo VEGF | 11.64 |
| endo bFGF | 0.00 |
| heart (T-1) ischemia | 17.02 |
| heart (T-14) non-obstructive DCM | 10.56 |
| heart (T-3399) DCM | 21.48 |
| BM stim | −1.30 |
| osteo dif | 1.20 |
| OA Cartilage (pool) | −4.66 |
| PBL HIV IIIB | −1.35 |
| MRC5 HSV strain F | −1.78 |
| W12 cells | −1.01 |

Gene Name sbg1563294bcadherin

Moderate overall expression. The highest normal expression is seen in the adrenal gland, whole brain, fetal brain, pancreas, hypothalamus, and the testis. Additional high expression is seen in the endometrium, esophagus, heart, liver, pancreas, rectum, and small intestine. This normal expression pattern suggests that this gene may be involved in certain metabolic disorders. The highest disease expression is seen in some of the colon, lung, and breast normal and tumor samples as well as in the asthmatic lung samples and the adenoid and tonsil. Large upregulation in 1 of 4 colon tumor samples is sufficient to make a claim for colon cancer. Upregulation in 1 of 4 lung tumor samples implicates this gene in cancer of the lung. Upregulation in 3 of 4 asthmatic lung samples coupled with immune cell expression in indicates a possible involvement in asthma. Upregulation in 3 of 3 disease heart samples implies an involvement in cardiovascular diseases such as non-obstructive and obstructive DCM and ischemia. Downregulation in the HIV-infected PBLs and the HSV-infected MRC5 cells suggests that this gene may be a host factor in HIV and HSV. This gene appears to show the same expression patterns in the disease samples as 1563294a. Based on this, it is likely that these two different primer sets are amplifying the same gene.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1563294b- | (sample | (sample | (sample | GOI | rRNA | rRNA | total |
| cadherin | 1 and 2) | 1) | 2) | Copies | (ng) | (ng) | RNA |

| | | | | | | | |
|---|---|---|---|---|---|---|---|
| Subcutaneous | 37.49, 35.73 | 1.36 | 4.27 | 2.82 | 3.06 | 16.34 | 46.00 |
| Adipocytes Zenbio | | | | | | | |
| Subcutaneous Adipose | 35.02, 36.17 | 6.8 | 3.22 | 5.01 | 0.96 | 52.36 | 262.30 |
| Zenbio | | | | | | | |
| Adrenal Gland Clontech | 33.03, 33.13 | 25.03 | 23.45 | 24.24 | 0.61 | 81.97 | 1986.89 |
| Whole Brain Clontech | 28.46, 28.38 | 494.81 | 521.62 | 508.22 | 7.24 | 6.91 | 3509.77 |
| Fetal Brain Clontech | 33.31, 33.77 | 20.82 | 15.42 | 18.12 | 0.48 | 103.95 | 1883.58 |
| Cerebellum Clontech | 34.14, 34 | 12.12 | 13.22 | 12.67 | 2.17 | 23.04 | 291.94 |
| Cervix | 34.5, 35.14 | 9.55 | 6.29 | 7.92 | 2.42 | 20.66 | 163.64 |
| Colon | 34.01, 33.67 | 13.2 | 16.39 | 14.80 | 2.71 | 18.45 | 272.97 |
| Endometrium | 34.01, 34.27 | 13.17 | 11.12 | 12.15 | 0.73 | 68.21 | 828.44 |
| Esophagus | 33.36, 33.24 | 20.16 | 21.74 | 20.95 | 1.37 | 36.50 | 764.60 |
| Heart Clontech | 33.64, 33.59 | 16.73 | 17.32 | 17.03 | 1.32 | 37.88 | 644.89 |
| Hypothalamus | 34.93, 33.8 | 7.24 | 15.11 | 11.18 | 0.32 | 155.28 | 1735.25 |
| Ileum | 34.29, 34.04 | 10.98 | 12.94 | 11.96 | 2.58 | 19.38 | 231.78 |
| Jejunum | 33.16, 35 | 22.96 | 6.91 | 14.94 | 6.60 | 7.58 | 113.14 |
| Kidney | 34.08, 34.26 | 12.61 | 11.2 | 11.91 | 2.12 | 23.58 | 280.78 |
| Liver | 33.38, 33.12 | 19.84 | 23.61 | 21.73 | 1.50 | 33.33 | 724.17 |
| Fetal Liver Clontech | 33.88, 33.28 | 14.33 | 21.14 | 17.74 | 10.40 | 4.81 | 85.26 |
| Lung | 33.73, 33.67 | 15.81 | 16.42 | 16.12 | 2.57 | 19.46 | 313.52 |
| Mammary Gland | 34.75, 34.81 | 8.1 | 7.79 | 7.95 | 13.00 | 3.85 | 30.56 |
| Clontech | | | | | | | |
| Myometrium | 33.89, 34.25 | 14.25 | 11.26 | 12.76 | 2.34 | 21.37 | 272.54 |
| Omentum | 34.44, 34.42 | 9.95 | 10.06 | 10.01 | 3.94 | 12.69 | 126.97 |
| Ovary | 33.31, 33.26 | 20.76 | 21.54 | 21.15 | 4.34 | 11.52 | 243.66 |
| Pancreas | 33.39, 33.86 | 19.76 | 14.5 | 17.13 | 0.81 | 61.80 | 1058.71 |
| Head of Pancreas | 34.03, 34.1 | 13.03 | 12.44 | 12.74 | 1.57 | 31.85 | 405.57 |
| Parotid Gland | 34.76, 34.14 | 8.09 | 12.07 | 10.08 | 5.48 | 9.12 | 91.97 |
| Placenta Clontech | 34.55, 33.51 | 9.24 | 18.23 | 13.74 | 5.26 | 9.51 | 130.56 |
| Prostate | 33.38, 33.86 | 19.9 | 14.52 | 17.21 | 3.00 | 16.67 | 286.83 |
| Rectum | 33.28, 33.69 | 21.26 | 16.22 | 18.74 | 1.23 | 40.65 | 761.79 |
| Salivary Gland Clontech | 34, 34.3 | 13.23 | 10.87 | 12.05 | 7.31 | 6.84 | 82.42 |
| Skeletal Muscle | 33.67, 33.98 | 16.45 | 13.38 | 14.92 | 1.26 | 39.68 | 591.87 |
| Clontech | | | | | | | |
| Skin | 34.09, 34.16 | 12.45 | 11.9 | 12.18 | 1.21 | 41.32 | 503.10 |
| Small Intestine Clontech | 33.85, 33.54 | 14.59 | 17.86 | 16.23 | 0.98 | 51.07 | 828.65 |
| Spleen | 33.41, 34.42 | 19.48 | 10.04 | 14.76 | 4.92 | 10.16 | 150.00 |
| Stomach | 33.27, 34.25 | 21.28 | 11.24 | 16.26 | 2.73 | 18.32 | 297.80 |
| Testis Clontech | 33.14, 33.18 | 23.23 | 22.66 | 22.95 | 0.57 | 87.87 | 2016.26 |
| Thymus Clontech | 31.43, 31.74 | 70.97 | 58.17 | 64.57 | 9.89 | 5.06 | 326.44 |
| Thyroid | 34.02, 33.55 | 13.09 | 17.78 | 15.44 | 2.77 | 18.05 | 278.61 |
| Trachea Clontech | 33.46, 33.58 | 18.81 | 17.38 | 18.10 | 9.71 | 5.15 | 93.18 |
| Urinary Bladder | 34.01, 33.99 | 13.16 | 13.32 | 13.24 | 5.47 | 9.14 | 121.02 |
| Uterus | 32.8, 33.11 | 29 | 23.72 | 26.36 | 5.34 | 9.36 | 246.82 |
| genomic | 27.37 | 1004.18 | | | | | |
| b-actin | 28.24 | 569.86 | | | | | |
| 1.00E+05 | 20.35 | 100000 | | | | | |
| 1.00E+05 | 20.42 | 100000 | | | | | |
| 1.00E+04 | 23.72 | 10000 | | | | | |
| 1.00E+04 | 23.91 | 10000 | | | | | |
| 1.00E+03 | 27.31 | 1000 | | | | | |
| 1.00E+03 | 27.29 | 1000 | | | | | |
| 1.00E+02 | 31.21 | 100 | | | | | |
| 1.00E+02 | 30.74 | 100 | | | | | |
| 1.00E+01 | 33.19 | −1 | | | | | |
| 1.00E+01 | 33.15 | −1 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1563294b- | (GSK | | GOI | total | | Disease |
| cadherin | identifier) | Ct | copies | RNA | Sample | Population |
| colon normal GW98-167 | 21941 | 30.36 | 297.32 | 594.64 | colon normal | |
| colon tumor GW98-166 | 21940 | 30.25 | 316.19 | 632.38 | colon tumor | 1.06 |
| colon normal GW98-178 | 22080 | 32.94 | 68.78 | 137.56 | colon normal | |
| colon tumor GW98-177 | 22060 | 31.16 | 189.21 | 378.42 | colon tumor | 2.75 |
| colon normal GW98-561 | 23514 | 31.86 | 126.77 | 253.54 | colon normal | |
| colon tumor GW98-560 | 23513 | 25.95 | 3631.23 | 7262.46 | colon tumor | 28.64 |
| colon normal GW98-894 | 24691 | 31.23 | 181.79 | 363.58 | colon normal | |
| colon tumor GW98-893 | 24690 | 32.39 | 93.93 | 187.86 | colon tumor | −1.94 |
| lung normal GW98-3 | 20742 | 26.77 | 2287.7 | 4575.40 | lung normal | |
| lung tumor GW98-2 | 20741 | 28.7 | 763.18 | 1526.36 | lung tumor | −3.00 |
| lung normal GW97-179 | 20677 | 29.12 | 600.92 | 1201.84 | lung normal | |
| lung tumor GW97-178 | 20676 | 26.39 | 2830.92 | 5661.84 | lung tumor | 4.71 |
| lung normal GW98-165 | 21922 | 27.18 | 1811.38 | 3622.76 | lung normal | |
| lung tumor GW98-164 | 21921 | 25.7 | 4187.41 | 8374.82 | lung tumor | 2.31 |
| lung normal GW98-282 | 22584 | 26.43 | 2772.29 | 5544.58 | lung normal | |
| lung tumor GW98-281 | 22583 | 29.05 | 624.92 | 1249.84 | lung tumor | −4.44 |
| breast normal GW00-392 | 28750 | 30.07 | 351.94 | 351.94 | breast normal | |
| breast tumor GW00-391 | 28746 | 30.38 | 294.08 | 588.16 | breast tumor | 1.67 |
| breast normal GW00-413 | 28798 | 31.2 | 185.02 | 185.02 | breast normal | |
| breast tumor GW00-412 | 28797 | 30.11 | 343.21 | 686.42 | breast tumor | 3.71 |
| breast normal GW00- | 27592-95 | 31.1 | 195.26 | 195.26 | breast normal | |
| 235:238 | | | | | | |
| breast tumor GW00- | 27588-91 | 30.83 | 228.5 | 228.50 | breast tumor | 1.17 |
| 231:234 | | | | | | |
| breast normal GW98-621 | 23656 | 30.07 | 350.88 | 701.76 | breast normal | |
| breast tumor GW98-620 | 23655 | 31.01 | 205.73 | 411.46 | breast tumor | −1.71 |
| brain normal BB99-542 | 25507 | 28.3 | 958.12 | 1916.24 | brain normal | |
| brain normal BB99-406 | 25509 | 27.98 | 1148.55 | 2297.10 | brain normal | |
| brain normal BB99-904 | 25546 | 30.32 | 304.77 | 609.54 | brain normal | |
| brain stage 5 ALZ BB99- | 25502 | 29.57 | 465.37 | 930.74 | brain stage 5 ALZ | −1.73 |
| 874 | | | | | | |
| brain stage 5 ALZ BB99- | 25503 | 27.37 | 1624.72 | 3249.44 | brain stage 5 ALZ | 2.02 |
| 887 | | | | | | |
| brain stage 5 ALZ BB99- | 25504 | 28.23 | 1000.08 | 2000.16 | brain stage 5 ALZ | 1.24 |
| 862 | | | | | | |
| brain stage 5 ALZ BB99- | 25542 | 28.66 | 783.14 | 1566.28 | brain stage 5 ALZ | −1.03 |
| 927 | | | | | | |
| CT lung | normal | 28.86 | 698.25 | 1396.50 | CT lung Nml | |
| lung 26 | normal | 29.32 | 536.67 | | lung 26 Nml | |
| lung 27 | normal | 32.23 | 102.91 | 102.91 | lung 27 Nml | |
| lung 24 | COPD | 32.82 | 73.51 | 73.51 | lung 24 COPD | −7.79 |
| lung 28 | COPD | 32.07 | 113.08 | 113.08 | lung 28 COPD | −5.06 |
| lung 23 | COPD | 30.17 | 330.88 | 330.88 | lung 23 COPD | −1.73 |
| lung 25 | normal | 30.91 | 217.57 | 217.57 | lung 25 Nml | |
| asthmatic lung | 29321 | 28.81 | 716.54 | 716.54 | asthmatic lung | 1.25 |
| ODO3112 | | | | | | |
| asthmatic lung | 29323 | 27.55 | 1463.97 | 2927.94 | asthmatic lung | 5.12 |
| ODO3433 | | | | | | |
| asthmatic lung | 29322 | 28.04 | 1112.34 | 2224.68 | asthmatic lung | 3.89 |
| ODO3397 | | | | | | |
| asthmatic lung | 29325 | 28.08 | 1084.62 | 2169.24 | asthmatic lung | 3.79 |
| ODO4928 | | | | | | |
| endo cells | control | 40 | 0 | 0.00 | endo cells | |
| endo VEGF | | 40 | 0 | 0.00 | endo VEGF | 0.00 |
| endo bFGF | | 40 | 0 | 0.00 | endo bFGF | 0.00 |
| heart Clontech | normal | 35.23 | 18.78 | 37.56 | heart | |
| heart (T-1) ischemic | 29417 | 31.14 | 191.53 | 383.06 | heart (T-1) | 10.20 |
| | | | | | ischemic | |
| heart (T-14) non- | 29422 | 31.71 | 138.24 | 276.48 | heart (T-14) non- | 7.36 |
| obstructive DCM | | | | | obstructive DCM | |
| heart (T-3399) DCM | 29426 | 30.96 | 211.99 | 423.98 | heart (T-3399) | 11.29 |
| | | | | | DCM | |
| adenoid GW99-269 | 26162 | 27.86 | 1230.52 | 2461.04 | adenoid | |
| tonsil GW98-280 | 22582 | 26.75 | 2315.5 | 4631.00 | tonsil | |
| T cells PC00314 | 28453 | 30.22 | 322.85 | 645.70 | T cells | |
| PBMNC | | 34.32 | 31.53 | 31.53 | PBMNC | |
| monocyte | | 32.88 | 71.25 | 142.50 | monocyte | |
| B cells PC00665 | 28455 | 30.67 | 250.35 | 500.70 | B cells | |
| dendritic cells | 28441 | 30.26 | 314.6 | 629.20 | dendritic cells | |
| neutrophils | 28440 | 28.51 | 851.25 | 851.25 | neutrophils | |
| eosinophils | 28446 | 30.2 | 326.41 | 652.82 | eosinophils | |
| BM unstim | | 31.78 | 133.15 | 133.15 | BM unstim | |
| BM stim | treated | 31.77 | 133.52 | 133.52 | BM stim | 1.00 |
| osteo dif | treated | 37.28 | 5.86 | 5.86 | osteo dif | −1.16 |
| osteo undif | | 37.03 | 6.77 | 6.77 | osteo undif | |
| chondrocytes | | 33.06 | 64.39 | 160.98 | chondrocytes | |
| OA Synovium IP12/01 | 29462 | 29.57 | 465.45 | 465.45 | OA Synovium | |
| OA Synovium NP10/01 | 29461 | 31.15 | 190.41 | 380.82 | OA Synovium | |
| OA Synovium NP57/00 | 28464 | 30.1 | 346.02 | 692.04 | OA Synovium | |
| RA Synovium | 28466 | 30.81 | 230.7 | 461.40 | RA Synovium | |
| NP03/01 | | | | | | |
| RA Synovium | 28467 | 29.68 | 438.8 | 877.60 | RA Synovium | |
| NP71/00 | | | | | | |
| RA Synovium | 28475 | 30.54 | 269.5 | 539.00 | RA Synovium | |
| NP45/00 | | | | | | |
| OA bone (biobank) | 29217 | 31.25 | 179.99 | 179.99 | OA bone | |
| | | | | | (biobank) | |
| OA bone Sample 1 | J. Emory | 30.88 | 222.01 | 444.02 | OA bone | |
| OA bone Sample 2 | J. Emory | 29.91 | 384.53 | 769.06 | OA bone | |
| Cartilage (pool) | Normal | 30.22 | 322.99 | 645.98 | Nml Cartilage | |
| | | | | | (pool) | |
| Cartilage (pool) | OA | 32.9 | 70.57 | 141.14 | OA Cartilage | −4.58 |
| | | | | | (pool) | |
| PBL unifected | 28441 | 30.47 | 280.4 | 560.80 | PBL unifected | |
| PBL HIV IIIB | 28442 | 31.96 | 120.33 | 240.66 | PBL HIV IIIB | −2.33 |
| MRC5 uninfected | 29158 | 34.5 | 28.48 | 56.96 | MRC5 uninfected | |
| (100%) | | | | | (100%) | |
| MRC5 HSV strain F | 29178 | 40 | 0 | 0.00 | MRC5 HSV | −56.96 |
| | | | | | strain F | |
| W12 cells | 29179 | 30.42 | 288.67 | 577.34 | W12 cells | 1.07 |
| Keratinocytes | 29180 | 30.54 | 268.96 | 537.92 | Keratinocytes | |
| B-actin control | | 27.74 | 1315.45 | | | |
| genomic | | 27.18 | 1811.29 | | | |
| 1.00E+05 | | 20.88 | 100000 | | | |
| 1.00E+05 | | 20.75 | 100000 | | | |
| 1.00E+04 | | 23.99 | 10000 | | | |
| 1.00E+04 | | 23.9 | 10000 | | | |
| 1.00E+03 | | 27.54 | 1000 | | | |
| 1.00E+03 | | 27.31 | 1000 | | | |
| 1.00E+02 | | 31.42 | 100 | | | |
| 1.00E+02 | | 32.09 | 100 | | | |
| 1.00E+01 | | 38.05 | 10 | | | |
| 1.00E+01 | | 40 | 0 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1563294bcadherin

| | Fold Change in Disease |
|---|---|
| | Population Relative to |
| Disease tissues | Normal |
| colon tumor | 1.06 |
| colon tumor | 2.75 |
| colon tumor | 28.64 |
| colon tumor | −1.94 |
| lung tumor | −3.00 |
| lung tumor | 4.71 |
| lung tumor | 2.31 |
| lung tumor | −4.44 |
| breast tumor | 1.67 |
| breast tumor | 3.71 |
| breast tumor | 1.17 |
| breast tumor | −1.71 |
| brain stage 5 ALZ | −1.73 |
| brain stage 5 ALZ | 2.02 |
| brain stage 5 ALZ | 1.24 |
| brain stage 5 ALZ | −1.03 |
| lung 24 COPD | −7.79 |
| lung 28 COPD | −5.06 |
| lung 23 COPD | −1.73 |
| asthmatic lung | 1.25 |
| asthmatic lung | 5.12 |
| asthmatic lung | 3.89 |
| asthmatic lung | 3.79 |
| endo VEGF | 0.00 |
| endo bFGF | 0.00 |
| heart (T-1) ischemic | 10.20 |
| heart (T-14) non-obstructive DCM | 7.36 |
| heart (T-3399) DCM | 11.29 |
| BM stim | 1.00 |
| osteo dif | −1.16 |
| OA Cartilage (pool) | −4.58 |
| PBL HIV IIIB | −2.33 |
| MRC5 HSV strain F | −56.96 |
| W12 cells | 1.07 |

Gene Name sbg1563509ECM

Moderate to high overall expression. This gene is expressed fairly ubiquitously throughout most of the normal tissues. The highest normal expression is seen in the lung, fetal liver, and mammary gland while the lowest normal expression is seen in the adrenal, fetal brain, hypothalamus, head of pancreas, skeletal muscle, and the testis. The highest disease expression is seen in some of the normal and tumor lung samples and in the OA and RA synovium samples. Downregulation in 1 of 4 lung tumor samples suggests a potential role in cancer of the lung. Upregulation of 1 of 4 breast tumor samples implicates this gene in breast cancer. Upregulation in 3 of 3 disease heart samples implies an involvement in cardiovascular diseases such as non-obstructive and obstructive DCM and ischemia. Downregulation in the HIV-infected PBLs and the HSV-infected MRC5 cells suggests that this gene may be a host factor in HIV and HSV. High expression in the OA and RA synovium samples with corroborating immune cell expression implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1563509ECM | 1 and 2) | 1) | 2) | Copies | (ng) | (ng) | RNA |

| | | | | | | | |
|---|---|---|---|---|---|---|---|
| Subcutaneous | 30.38, 30.22 | 142.77 | 157.17 | 149.97 | 3.06 | 16.34 | 2450.49 |
| Adipocytes Zenbio | | | | | | | |
| Subcutaneous Adipose | 33.18, 33.06 | 25.38 | 27.32 | 26.35 | 0.96 | 52.36 | 1379.58 |
| Zenbio | | | | | | | |
| Adrenal Gland Clontech | 40, 40 | 0 | 0.66 | 0.33 | 0.61 | 81.97 | 27.05 |
| Whole Brain Clontech | 28.64, 28.64 | 416.91 | 417.36 | 417.14 | 7.24 | 6.91 | 2880.77 |
| Fetal Brain Clontech | 40, 40 | 0.61 | 0 | 0.31 | 0.48 | 103.95 | 31.70 |
| Cerebellum Clontech | 40, 37.11 | 0 | 2.26 | 1.13 | 2.17 | 23.04 | 26.04 |
| Cervix | 31.79, 31.96 | 59.71 | 53.85 | 56.78 | 2.42 | 20.66 | 1173.14 |
| Colon | 32.07, 31.6 | 50.27 | 67.25 | 58.76 | 2.71 | 18.45 | 1084.13 |
| Endometrium | 33.39, 35.81 | 22.3 | 5.02 | 13.66 | 0.73 | 68.21 | 931.79 |
| Esophagus | 32.84, 33.08 | 31.34 | 27.05 | 29.20 | 1.37 | 36.50 | 1065.51 |
| Heart Clontech | 32.16, 32.92 | 47.54 | 29.78 | 38.66 | 1.32 | 37.88 | 1464.39 |
| Hypothalamus | 40, 40 | 0 | 0 | 0.00 | 0.32 | 155.28 | 0.00 |
| Ileum | 30.79, 30.95 | 111.17 | 100.57 | 105.87 | 2.58 | 19.38 | 2051.74 |
| Jejunum | 30.98, 30.35 | 98.76 | 145.23 | 122.00 | 6.60 | 7.58 | 924.20 |
| Kidney | 33.39, 32.68 | 22.36 | 34.55 | 28.46 | 2.12 | 23.58 | 671.11 |
| Liver | 33.58, 32.98 | 19.81 | 28.77 | 24.29 | 1.50 | 33.33 | 809.67 |
| Fetal Liver Clontech | 27.68, 27.62 | 752.33 | 780.99 | 766.66 | 10.40 | 4.81 | 3685.87 |
| Lung | 29.72, 29.24 | 214.26 | 288.46 | 251.36 | 2.57 | 19.46 | 4890.27 |
| Mammary Gland | 27.58, 27.41 | 802.77 | 889.8 | 846.29 | 13.00 | 3.85 | 3254.94 |
| Clontech | | | | | | | |
| Myometrium | 31.3, 31.26 | 80.88 | 82.8 | 81.84 | 2.34 | 21.37 | 1748.72 |
| Omentum | 31.16, 30.49 | 88.32 | 133.39 | 110.86 | 3.94 | 12.69 | 1406.79 |
| Ovary | 29.81, 29.17 | 202.59 | 300.28 | 251.44 | 4.34 | 11.52 | 2896.72 |
| Pancreas | 34.04, 34.58 | 14.94 | 10.75 | 12.85 | 0.81 | 61.80 | 793.88 |
| Head of Pancreas | 40, 40 | 0 | 0 | 0.00 | 1.57 | 31.85 | 0.00 |
| Parotid Gland | 30.91, 31.08 | 102.7 | 92.68 | 97.69 | 5.48 | 9.12 | 891.33 |
| Placenta Clontech | 31.84, 31.93 | 58.07 | 54.86 | 56.47 | 5.26 | 9.51 | 536.74 |
| Prostate | 34.37, 34.51 | 12.24 | 11.17 | 11.71 | 3.00 | 16.67 | 195.08 |
| Rectum | 32.07, 32.38 | 50.41 | 41.58 | 46.00 | 1.23 | 40.65 | 1869.72 |
| Salivary Gland Clontech | 32.14, 31.5 | 48.38 | 71.44 | 59.91 | 7.31 | 6.84 | 409.78 |
| Skeletal Muscle | 40, 34.72 | 0 | 9.81 | 4.91 | 1.26 | 39.68 | 194.64 |
| Clontech | | | | | | | |
| Skin | 34.2, 33.36 | 13.55 | 22.72 | 18.14 | 1.21 | 41.32 | 749.38 |
| Small Intestine Clontech | 40, 35.18 | 0.59 | 7.41 | 4.00 | 0.98 | 51.07 | 204.29 |
| Spleen | 30.31, 30.31 | 149.25 | 149.43 | 149.34 | 4.92 | 10.16 | 1517.68 |
| Stomach | 33.04, 33.16 | 27.79 | 25.75 | 26.77 | 2.73 | 18.32 | 490.29 |
| Testis Clontech | 40, 40 | 0.94 | 0.74 | 0.84 | 0.57 | 87.87 | 73.81 |
| Thymus Clontech | 29.38, 29.2 | 263.89 | 296.27 | 280.08 | 9.89 | 5.06 | 1415.98 |
| Thyroid | 31.72, 31.9 | 62.48 | 55.9 | 59.19 | 2.77 | 18.05 | 1068.41 |
| Trachea Clontech | 30.31, 29.88 | 148.77 | 194.55 | 171.66 | 9.71 | 5.15 | 883.93 |
| Urinary Bladder | 32.11, 31.47 | 49.26 | 72.88 | 61.07 | 5.47 | 9.14 | 558.23 |
| Uterus | 31.42, 30.77 | 75.27 | 112.11 | 93.69 | 5.34 | 9.36 | 877.25 |
| genomic | 26.84 | 1263.86 | | | | | |
| b-actin | 28.08 | 588.35 | | | | | |
| 1.00E+05 | 19.86 | 100000 | | | | | |
| 1.00E+05 | 20 | 100000 | | | | | |
| 1.00E+04 | 23.16 | 10000 | | | | | |
| 1.00E+04 | 23.17 | 10000 | | | | | |
| 1.00E+03 | 26.87 | 1000 | | | | | |
| 1.00E+03 | 26.65 | 1000 | | | | | |
| 1.00E+02 | 32.21 | 100 | | | | | |
| 1.00E+02 | 32.05 | 100 | | | | | |
| 1.00E+01 | 40 | 10 | | | | | |
| 1.00E+01 | 33.56 | 10 | | | | | |
| 1.00E+00 | 40 | 1 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1563509ECM | identifier) | Ct | copies | RNA | Sample | Population |
| colon normal GW98-167 | 21941 | 26.32 | 2332.21 | 4664.42 | colon normal | |
| colon tumor GW98-166 | 21940 | 25.44 | 3916.42 | 7832.84 | colon tumor | 1.68 |
| colon normal GW98-178 | 22080 | 28.98 | 482.04 | 964.08 | colon normal | |
| colon tumor GW98-177 | 22060 | 29.93 | 275.13 | 550.26 | colon tumor | −1.75 |
| colon normal GW98-561 | 23514 | 29.66 | 322.03 | 644.06 | colon normal | |
| colon tumor GW98-560 | 23513 | 28.47 | 651.69 | 1303.38 | colon tumor | 2.02 |
| colon normal GW98-894 | 24691 | 28.51 | 635.18 | 1270.36 | colon normal | |
| colon tumor GW98-893 | 24690 | 29.67 | 320.97 | 641.94 | colon tumor | −1.98 |
| lung normal GW98-3 | 20742 | 23.5 | 12376.95 | 24753.90 | lung normal | |
| lung tumor GW98-2 | 20741 | 29.25 | 409.4 | 818.80 | lung tumor | −30.23 |
| lung normal GW97-179 | 20677 | 25.12 | 4727.38 | 9454.76 | lung normal | |
| lung tumor GW97-178 | 20676 | 23.65 | 11323.07 | 22646.14 | lung tumor | 2.40 |
| lung normal GW98-165 | 21922 | 24.12 | 8559.3 | 17118.60 | lung normal | |
| lung tumor GW98-164 | 21921 | 24.13 | 8529.62 | 17059.24 | lung tumor | −1.00 |
| lung normal GW98-282 | 22584 | 26.18 | 2520.39 | 5040.78 | lung normal | |
| lung tumor GW98-281 | 22583 | 24.28 | 7787.44 | 15574.88 | lung tumor | 3.09 |
| breast normal GW00-392 | 28750 | 25.24 | 4414.62 | 4414.62 | breast normal | |
| breast tumor GW00-391 | 28746 | 26.18 | 2521.92 | 5043.84 | breast tumor | 1.14 |
| breast normal GW00-413 | 28798 | 28 | 859.75 | 859.75 | breast normal | |
| breast tumor GW00-412 | 28797 | 28.18 | 773.07 | 1546.14 | breast tumor | 1.80 |
| breast normal GW00- | 27592-95 | 29.09 | 451.71 | 451.71 | breast normal | |
| 235:238 | | | | | | |
| breast tumor GW00- | 27588-91 | 25.21 | 4489.31 | 4489.31 | breast tumor | 9.94 |
| 231:234 | | | | | | |
| breast normal GW98-621 | 23656 | 24.01 | 9115.61 | 18231.22 | breast normal | |
| breast tumor GW98-620 | 23655 | 26.31 | 2333.67 | 4667.34 | breast tumor | −3.91 |
| brain normal BB99-542 | 25507 | 31.35 | 118.13 | 236.26 | brain normal | |
| brain normal BB99-406 | 25509 | 31.59 | 103.02 | 206.04 | brain normal | |
| brain normal BB99-904 | 25546 | 32.23 | 70.46 | 140.92 | brain normal | |
| brain stage 5 ALZ BB99- | 25502 | 31.03 | 143.12 | 286.24 | brain stage 5 ALZ | 1.47 |
| 874 | | | | | | |
| brain stage 5 ALZ BB99- | 25503 | 30.04 | 257.7 | 515.40 | brain stage 5 ALZ | 2.65 |
| 887 | | | | | | |
| brain stage 5 ALZ BB99- | 25504 | 30.68 | 176.24 | 352.48 | brain stage 5 ALZ | 1.81 |
| 862 | | | | | | |
| brain stage 5 ALZ BB99- | 25542 | 30.85 | 158.78 | 317.56 | brain stage 5 ALZ | 1.63 |
| 927 | | | | | | |
| CT lung | normal | 26.51 | 2080.42 | 4160.84 | CT lung Nml | |
| lung 26 | normal | 27.53 | 1136.99 | | lung 26 Nml | |
| lung 27 | normal | 27.62 | 1076.86 | 1076.86 | lung 27 Nml | |
| lung 24 | COPD | 27.67 | 1044.37 | 1044.37 | lung 24 COPD | −2.14 |
| lung 28 | COPD | 27.31 | 1293.45 | 1293.45 | lung 28 COPD | −1.73 |
| lung 23 | COPD | 27.73 | 1008.19 | 1008.19 | lung 23 COPD | −2.22 |
| lung 25 | normal | 27.09 | 1476.73 | 1476.73 | lung 25 Nml | |
| asthmatic lung ODO3112 | 29321 | 28.59 | 607.37 | 607.37 | asthmatic lung | −3.68 |
| asthmatic lung ODO3433 | 29323 | 26.78 | 1774.99 | 3549.98 | asthmatic lung | 1.59 |
| asthmatic lung ODO3397 | 29322 | 26 | 2814.56 | 5629.12 | asthmatic lung | 2.52 |
| asthmatic lung ODO4928 | 29325 | 26.69 | 1867.05 | 3734.10 | asthmatic lung | 1.67 |
| endo cells | control | 29.39 | 377.18 | 377.18 | endo cells | |
| endo VEGF | | 29.2 | 422.38 | 422.38 | endo VEGF | 1.12 |
| endo bFGF | | 30.87 | 157.65 | 157.65 | endo bFGF | −2.39 |
| heart Clontech | normal | 30.26 | 225.09 | 450.18 | heart | |
| heart (T-1) ischemic | 29417 | 26.68 | 1885.2 | 3770.40 | heart (T-1) | 8.38 |
| | | | | | ischemic | |
| heart (T-14) non- | 29422 | 26.44 | 2160.24 | 4320.48 | heart (T-14) non- | 9.60 |
| obstructive DCM | | | | | obstructive DCM | |
| heart (T-3399) DCM | 29426 | 26.04 | 2749.86 | 5499.72 | heart (T-3399) | 12.22 |
| | | | | | DCM | |
| adenoid GW99-269 | 26162 | 28.03 | 842.76 | 1685.52 | adenoid | |
| tonsil GW98-280 | 22582 | 27.99 | 867.51 | 1735.02 | tonsil | |
| T cells PC00314 | 28453 | 35.16 | 12.44 | 24.88 | T cells | |
| PBMNC | | 40 | 0 | 0.00 | PBMNC | |
| monocyte | | 35.75 | 8.76 | 17.52 | monocyte | |
| B cells PC00665 | 28455 | 36.19 | 6.74 | 13.48 | B cells | |
| dendritic cells | 28441 | 40 | 0 | 0.00 | dendritic cells | |
| neutrophils | 28440 | 33.4 | 35.17 | 35.17 | neutrophils | |
| eosinophils | 28446 | 34.21 | 21.78 | 43.56 | eosinophils | |
| BM unstim | | 35.58 | 9.7 | 9.70 | BM unstim | |
| BM stim | treated | 34.26 | 21.08 | 21.08 | BM stim | 2.17 |
| osteo dif | treated | 29.54 | 345.54 | 345.54 | osteo dif | −1.27 |
| osteo undif | | 29.14 | 438.01 | 438.01 | osteo undif | |
| chondrocytes | | 26.03 | 2766.86 | 6917.15 | chondrocytes | |
| OA Synovium IP12/01 | 29462 | 24.15 | 8414.27 | 8414.27 | OA Synovium | |
| OA Synovium NP10/01 | 29461 | 25.17 | 4607.95 | 9215.90 | OA Synovium | |
| OA Synovium NP57/00 | 28464 | 23.55 | 11981.42 | 23962.84 | OA Synovium | |
| RA Synovium | 28466 | 24.98 | 5127.1 | 10254.20 | RA Synovium | |
| NP03/01 | | | | | | |
| RA Synovium | 28467 | 24.22 | 8086.53 | 16173.06 | RA Synovium | |
| NP71/00 | | | | | | |
| RA Synovium | 28475 | 25.08 | 4839.85 | 9679.70 | RA Synovium | |
| NP45/00 | | | | | | |
| OA bone (biobank) | 29217 | 28.59 | 605.78 | 605.78 | OA bone | |
| | | | | | (biobank) | |
| OA bone Sample 1 | J. Emory | 27.67 | 1043.14 | 2086.28 | OA bone | |
| OA bone Sample 2 | J. Emory | 27.8 | 969.07 | 1938.14 | OA bone | |
| Cartilage (pool) | Normal | 26.06 | 2714.12 | 5428.24 | Nml Cartilage | |
| | | | | | (pool) | |
| Cartilage (pool) | OA | 26.45 | 2156.8 | 4313.60 | OA Cartilage | −1.26 |
| | | | | | (pool) | |
| PBL unifected | 28441 | 29.88 | 282.45 | 564.90 | PBL unifected | |
| PBL HIV IIIB | 28442 | 34.32 | 20.4 | 40.80 | PBL HIV IIIB | −13.85 |
| MRC5 uninfected | 29158 | 29.36 | 385.52 | 771.04 | MRC5 | |
| (100%) | | | | | uninfected | |
| | | | | | (100%) | |
| MRC5 HSV strain F | 29178 | 32.49 | 60.36 | 120.72 | MRC5 HSV | −6.39 |
| | | | | | strain F | |
| W12 cells | 29179 | 32.1 | 75.78 | 151.56 | W12 cells | −1.30 |
| Keratinocytes | 29180 | 31.66 | 98.45 | 196.90 | Keratinocytes | |
| B-actin control | | 28.06 | 828.85 | | | |
| genomic | | 27.22 | 1362.28 | | | |
| 1.00E+05 | | 20.1 | 100000 | | | |
| 1.00E+05 | | 20.32 | 100000 | | | |
| 1.00E+04 | | 23.72 | 10000 | | | |
| 1.00E+04 | | 23.67 | 10000 | | | |
| 1.00E+03 | | 27.61 | 1000 | | | |
| 1.00E+03 | | 27.29 | 1000 | | | |
| 1.00E+02 | | 32.12 | 100 | | | |
| 1.00E+02 | | 31.32 | 100 | | | |
| 1.00E+01 | | 40 | 0 | | | |
| 1.00E+01 | | 35.78 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1563509ECM

| | Fold Change in Disease |
|---|---|
| | Population Relative to |
| Disease tissues | Normal |
| colon tumor | 1.68 |
| colon tumor | −1.75 |
| colon tumor | 2.02 |
| colon tumor | −1.98 |
| lung tumor | −30.23 |
| lung tumor | 2.40 |
| lung tumor | −1.00 |
| lung tumor | 3.09 |
| breast tumor | 1.14 |
| breast tumor | 1.80 |
| breast tumor | 9.94 |
| breast tumor | −3.91 |
| brain stage 5 ALZ | 1.47 |
| brain stage 5 ALZ | 2.65 |
| brain stage 5 ALZ | 1.81 |
| brain stage 5 ALZ | 1.63 |
| lung 24 COPD | −2.14 |
| lung 28 COPD | −1.73 |
| lung 23 COPD | −2.22 |
| asthmatic lung | −3.68 |
| asthmatic lung | 1.59 |
| asthmatic lung | 2.52 |
| asthmatic lung | 1.67 |
| endo VEGF | 1.12 |
| endo bFGF | −2.39 |
| heart (T-1) ischemic | 8.38 |
| heart (T-14) non-obstructive DCM | 9.60 |
| heart (T-3399) DCM | 12.22 |
| BM stim | 2.17 |
| osteo dif | −1.27 |
| OA Cartilage (pool) | −1.26 |
| PBL HIV IIIB | −13.85 |
| MRC5 HSV strain F | −6.39 |
| W12 cells | −1.30 |

Gene Name sbg1552159Serprotease

Moderate overall expression. The highest normal expression is seen in the whole brain, fetal brain, thymus, and testis. The highest disease expression is seen in the some of the normal counterparts of both the lung and breast tumor samples as well as in three of the asthmatic lung samples. Significant downregulation in 1 of 4 lung tumor samples and 1 of 4 breast tumor samples is sufficient to claim involvement in lung and breast cancer. Upregulation in 3 of 4 Alzheimer's brain samples suggests an involvement in Alzheimer's disease. Upregulation in 3 of 4 asthmatic lung samples with corroborating high expression in the neutrophils indicates a possible involvement in asthma. Upregulated in the differentiated osteoblasts. Downregulation in the HIV-infected PBLs and the HSV-infected MRC5 cells suggests that this gene may be a host factor in HIV and HSV. Downregulation in the W12 cells suggests that this gene may be a host factor in HPV.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1552159- | (sample | (sample | (sample | GOI | rRNA | rRNA | total |
| Serprotease | 1 and 2) | 1) | 2) | Copies | (ng) | (ng) | RNA |
| Subcutaneous | 36.04, 34.59 | 10.17 | 23.6 | 16.89 | 3.06 | 16.34 | 275.90 |
| Adipocytes Zenbio | | | | | | | |
| Subcutaneous Adipose | 40, 40 | 0.16 | 0 | 0.08 | 0.96 | 52.36 | 4.19 |
| Zenbio | | | | | | | |
| Adrenal Gland Clontech | 40, 40 | 0 | 0 | 0.00 | 0.61 | 81.97 | 0.00 |
| Whole Brain Clontech | 27.89, 27.71 | 1131.62 | 1253.56 | 1192.59 | 7.24 | 6.91 | 8236.12 |
| Fetal Brain Clontech | 36.08, 35.62 | 9.95 | 13.02 | 11.49 | 0.48 | 103.95 | 1193.87 |
| Cerebellum Clontech | 37.52, 40 | 4.34 | 0 | 2.17 | 2.17 | 23.04 | 50.00 |
| Cervix | 36.03, 37.08 | 10.22 | 5.6 | 7.91 | 2.42 | 20.66 | 163.43 |
| Colon | 34.81, 40 | 20.71 | 0 | 10.36 | 2.71 | 18.45 | 191.05 |
| Endometrium | 40, 40 | 0 | 0 | 0.00 | 0.73 | 68.21 | 0.00 |
| Esophagus | 36.79 | 6.59 | 0 | 3.30 | 1.37 | 36.50 | 120.26 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 36.49, 36.39 | 7.84 | 8.33 | 8.09 | 2.58 | 19.38 | 156.69 |
| Jejunum | 32.83, 34.02 | 65.26 | 32.68 | 48.97 | 6.60 | 7.58 | 370.98 |
| Kidney | 36.08, 35.48 | 9.96 | 14.12 | 12.04 | 2.12 | 23.58 | 283.96 |
| Liver | 35.97, 40 | 10.62 | 20.57 | 15.60 | 1.50 | 33.33 | 519.83 |
| Fetal Liver Clontech | 33.01, 33.66 | 58.78 | 40.39 | 49.59 | 10.40 | 4.81 | 238.39 |
| Lung | 33.21, 33.19 | 52.28 | 52.86 | 52.57 | 2.57 | 19.46 | 1022.76 |
| Mammary Gland | 32.97, 32.13 | 60.1 | 97.47 | 78.79 | 13.00 | 3.85 | 303.02 |
| Clontech | | | | | | | |
| Myometrium | 34.29, 33.57 | 28.1 | 42.41 | 35.26 | 2.34 | 21.37 | 753.31 |
| Omentum | 40, 35.08 | 0 | 17.79 | 8.90 | 3.94 | 12.69 | 112.88 |
| Ovary | 34.1, 33.78 | 31.22 | 37.67 | 34.45 | 4.34 | 11.52 | 396.83 |
| Pancreas | 36.9, 40 | 6.21 | 0 | 3.11 | 0.81 | 61.80 | 191.90 |
| Head of Pancreas | 40, 35.98 | 0 | 10.58 | 5.29 | 1.57 | 31.85 | 168.47 |
| Parotid Gland | 36.02, 36.29 | 10.33 | 8.8 | 9.57 | 5.48 | 9.12 | 87.27 |
| Placenta Clontech | 35.77, 40 | 11.89 | 0 | 5.95 | 5.26 | 9.51 | 56.51 |
| Prostate | 40, 36.13 | 0 | 9.7 | 4.85 | 3.00 | 16.67 | 80.83 |
| Rectum | 40, 40 | 0 | 0 | 0.00 | 1.23 | 40.65 | 0.00 |
| Salivary Gland Clontech | 39.51, 35.16 | 1.37 | 16.91 | 9.14 | 7.31 | 6.84 | 62.52 |
| Skeletal Muscle | 36.29, 36.68 | 8.82 | 7.05 | 7.94 | 1.26 | 39.68 | 314.88 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 33.79, 35.77 | 37.52 | 11.89 | 24.71 | 4.92 | 10.16 | 251.07 |
| Stomach | 37.88, 37.81 | 3.53 | 3.67 | 3.60 | 2.73 | 18.32 | 65.93 |
| Testis Clontech | 35.14, 34.77 | 17.12 | 21.25 | 19.19 | 0.57 | 87.87 | 1685.85 |
| Thymus Clontech | 29.65, 29.15 | 409.27 | 547.64 | 478.46 | 9.89 | 5.06 | 2418.88 |
| Thyroid | 36.32, 40 | 8.64 | 0 | 4.32 | 2.77 | 18.05 | 77.98 |
| Trachea Clontech | 32.97, 35.24 | 60.19 | 16.17 | 38.18 | 9.71 | 5.15 | 196.60 |
| Urinary Bladder | 36.78, 36.33 | 6.65 | 8.61 | 7.63 | 5.47 | 9.14 | 69.74 |
| Uterus | 33.46, 33.09 | 45.17 | 55.91 | 50.54 | 5.34 | 9.36 | 473.22 |
| genomic | 27.52 | 1400.32 | | | | | |
| b-actin | 28.07 | 1019.35 | | | | | |
| 1.00E+05 | 20.41 | 100000 | | | | | |
| 1.00E+05 | 20.42 | 100000 | | | | | |
| 1.00E+04 | 23.86 | 10000 | | | | | |
| 1.00E+04 | 24.03 | 10000 | | | | | |
| 1.00E+03 | 27.72 | 1000 | | | | | |
| 1.00E+03 | 27.91 | 1000 | | | | | |
| 1.00E+02 | 32 | 100 | | | | | |
| 1.00E+02 | 32.12 | 100 | | | | | |
| 1.00E+01 | 36.2 | 10 | | | | | |
| 1.00E+01 | 36.36 | 10 | | | | | |
| 1.00E+00 | 40 | 0 | | | | | |
| 1.00E+00 | 40 | 0 | | | | | |
| NTC | 40 | −1 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1552159- | (GSK | | GOI | total | | Disease |
| Serprotease | identifier) | Ct | copies | RNA | Sample | Population |
| colon normal GW98-167 | 21941 | 31.77 | 108.85 | 217.70 | colon normal | |
| colon tumor GW98-166 | 21940 | 32.95 | 56.73 | 113.46 | colon tumor | −1.92 |
| colon normal GW98-178 | 22080 | 34.14 | 29.37 | 58.74 | colon normal | |
| colon tumor GW98-177 | 22060 | 33.79 | 35.61 | 71.22 | colon tumor | 1.21 |
| colon normal GW98-561 | 23514 | 31.99 | 96.4 | 192.80 | colon normal | |
| colon tumor GW98-560 | 23513 | 31.61 | 119.45 | 238.90 | colon tumor | 1.24 |
| colon normal GW98-894 | 24691 | 32.6 | 68.97 | 137.94 | colon normal | |
| colon tumor GW98-893 | 24690 | 33.77 | 36.1 | 72.20 | colon tumor | −1.91 |
| lung normal GW98-3 | 20742 | 27.73 | 1017.76 | 2035.52 | lung normal | |
| lung tumor GW98-2 | 20741 | 34.28 | 27.21 | 54.42 | lung tumor | −37.40 |
| lung normal GW97-179 | 20677 | 31.15 | 153.55 | 307.10 | lung normal | |
| lung tumor GW97-178 | 20676 | 29.11 | 474.86 | 949.72 | lung tumor | 3.09 |
| lung normal GW98-165 | 21922 | 27.22 | 1351.96 | 2703.92 | lung normal | |
| lung tumor GW98-164 | 21921 | 29.62 | 357.43 | 714.86 | lung tumor | −3.78 |
| lung normal GW98-282 | 22584 | 30.8 | 186.98 | 373.96 | lung normal | |
| lung tumor GW98-281 | 22583 | 28.49 | 670.41 | 1340.82 | lung tumor | 3.59 |
| breast normal GW00-392 | 28750 | 30.06 | 280.28 | 280.28 | breast normal | |
| breast tumor GW00-391 | 28746 | 28.92 | 526.38 | 1052.76 | breast tumor | 3.76 |
| breast normal GW00-413 | 28798 | 31.84 | 104.84 | 104.84 | breast normal | |
| breast tumor GW00-412 | 28797 | 32.12 | 90.02 | 180.04 | breast tumor | 1.72 |
| breast normal GW00- | 27592-95 | 33.35 | 45.65 | 45.65 | breast normal | |
| 235:238 | | | | | | |
| breast tumor GW00- | 27588-91 | 31.57 | 121.62 | 121.62 | breast tumor | 2.66 |
| 231:234 | | | | | | |
| breast normal GW98-621 | 23656 | 28.87 | 542.39 | 1084.78 | breast normal | |
| breast tumor GW98-620 | 23655 | 33.91 | 33.4 | 66.80 | breast tumor | −16.24 |
| brain normal BB99-542 | 25507 | 29.48 | 386.14 | 772.28 | brain normal | |
| brain normal BB99-406 | 25509 | 29.99 | 291.64 | 583.28 | brain normal | |
| brain normal BB99-904 | 25546 | 31.2 | 149.66 | 299.32 | brain normal | |
| brain stage 5 ALZ BB99- | 25502 | 30.16 | 265.84 | 531.68 | brain stage 5 ALZ | −1.04 |
| 874 | | | | | | |
| brain stage 5 ALZ BB99- | 25503 | 28.08 | 840 | 1680.00 | brain stage 5 ALZ | 3.05 |
| 887 | | | | | | |
| brain stage 5 ALZ BB99- | 25504 | 28.53 | 655.61 | 1311.22 | brain stage 5 ALZ | 2.38 |
| 862 | | | | | | |
| brain stage 5 ALZ BB99- | 25542 | 28.08 | 837.24 | 1674.48 | brain stage 5 ALZ | 3.04 |
| 927 | | | | | | |
| CT lung | normal | 30.05 | 282.31 | 564.62 | CT lung Nml | |
| lung 26 | normal | 30.85 | 181.53 | | lung 26 Nml | |
| lung 27 | normal | 33.45 | 43.19 | 43.19 | lung 27 Nml | |
| lung 24 | COPD | 31.01 | 166.35 | 166.35 | lung 24 COPD | −1.34 |
| lung 28 | COPD | 32.83 | 60.69 | 60.69 | lung 28 COPD | −3.68 |
| lung 23 | COPD | 32.14 | 88.79 | 88.79 | lung 23 COPD | −2.52 |
| lung 25 | normal | 32.77 | 62.65 | 62.65 | lung 25 Nml | |
| asthmatic lung ODO3112 | 29321 | 32.77 | 62.83 | 62.83 | asthmatic lung | −3.56 |
| asthmatic lung ODO3433 | 29323 | 27.59 | 1100.3 | 2200.60 | asthmatic lung | 9.85 |
| asthmatic lung ODO3397 | 29322 | 27.93 | 914.58 | 1829.16 | asthmatic lung | 8.18 |
| asthmatic lung ODO4928 | 29325 | 29.21 | 449.51 | 899.02 | asthmatic lung | 4.02 |
| endo cells | control | 36.25 | 9.18 | 9.18 | endo cells | |
| endo VEGF | | 35.45 | 14.27 | 14.27 | endo VEGF | 1.55 |
| endo bFGF | | 36 | 10.49 | 10.49 | endo bFGF | 1.14 |
| heart Clontech | normal | 32.44 | 75.33 | 150.66 | heart | |
| heart (T-1) ischemic | 29417 | 32.2 | 86 | 172.00 | heart (T-1) | 1.14 |
| | | | | | ischemic | |
| heart (T-14) non- | 29422 | 31.72 | 112.13 | 224.26 | heart (T-14) non- | 1.49 |
| obstructive DCM | | | | | obstructive DCM | |
| heart (T-3399) DCM | 29426 | 32.01 | 95.37 | 190.74 | heart (T-3399) | 1.27 |
| | | | | | DCM | |
| adenoid GW99-269 | 26162 | 32.57 | 70.26 | 140.52 | adenoid | |
| tonsil GW98-280 | 22582 | 32.18 | 87.15 | 174.30 | tonsil | |
| T cells PC00314 | 28453 | 36.33 | 8.79 | 17.58 | T cells | |
| PBMNC | | 35.19 | 16.42 | 16.42 | PBMNC | |
| monocyte | | 39.03 | 1.97 | 3.94 | monocyte | |
| B cells PC00665 | 28455 | 33.31 | 46.62 | 93.24 | B cells | |
| dendritic cells | 28441 | 30.84 | 182.9 | 365.80 | dendritic cells | |
| neutrophils | 28440 | 29.11 | 475.18 | 475.18 | neutrophils | |
| eosinophils | 28446 | 32.09 | 91.35 | 182.70 | eosinophils | |
| BM unstim | | 32.79 | 62.06 | 62.06 | BM unstim | |
| BM stim | treated | 35 | 18.25 | 18.25 | BM stim | −3.40 |
| osteo dif | treated | 32.61 | 68.38 | 68.38 | osteo dif | 9.29 |
| osteo undif | | 36.65 | 7.36 | 7.36 | osteo undif | |
| chondrocytes | | 33.66 | 38.41 | 96.03 | chondrocytes | |
| OA Synovium IP12/01 | 29462 | 29.28 | 431.45 | 431.45 | OA Synovium | |
| OA Synovium NP10/01 | 29461 | 31.12 | 156.26 | 312.52 | OA Synovium | |
| OA Synovium NP57/00 | 28464 | 30.2 | 260.25 | 520.50 | OA Synovium | |
| RA Synovium | 28466 | 31.27 | 143.47 | 286.94 | RA Synovium | |
| NP03/01 | | | | | | |
| RA Synovium | 28467 | 29.55 | 372.02 | 744.04 | RA Synovium | |
| NP71/00 | | | | | | |
| RA Synovium | 28475 | 31.04 | 162.93 | 325.86 | RA Synovium | |
| NP45/00 | | | | | | |
| OA bone (biobank) | 29217 | 31.88 | 102.61 | 102.61 | OA bone (biobank) | |
| OA bone Sample 1 | J. Emory | 29.15 | 463.55 | 927.10 | OA bone | |
| OA bone Sample 2 | J. Emory | 32.22 | 85.12 | 170.24 | OA bone | |
| Cartilage (pool) | Normal | 29.1 | 477.1 | 954.20 | Nml Cartilage | |
| | | | | | (pool) | |
| Cartilage (pool) | OA | 31.42 | 132.47 | 264.94 | OA Cartilage (pool) | −3.60 |
| PBL unifected | 28441 | 35.78 | 11.86 | 23.72 | PBL unifected | |
| PBL HIV IIIB | 28442 | 40 | 0 | 0.00 | PBL HIV IIIB | −23.72 |
| MRC5 uninfected | 29158 | 29.8 | 323.53 | 647.06 | MRC5 uninfected | |
| (100%) | | | | | (100%) | |
| MRC5 HSV strain F | 29178 | 32.5 | 72.82 | 145.64 | MRC5 HSV strain | −4.44 |
| | | | | | F | |
| W12 cells | 29179 | 34.63 | 22.39 | 44.78 | W12 cells | −8.73 |
| Keratinocytes | 29180 | 30.72 | 195.4 | 390.80 | Keratinocytes | |
| B-actin control | | 27.32 | 1277.66 | | | |
| genomic | | 27.01 | 1519.42 | | | |
| 1.00E+05 | | 19.85 | 100000 | | | |
| 1.00E+05 | | 19.83 | 100000 | | | |
| 1.00E+04 | | 23.23 | 10000 | | | |
| 1.00E+04 | | 23.32 | 10000 | | | |
| 1.00E+03 | | 27.27 | 1000 | | | |
| 1.00E+03 | | 27.11 | 1000 | | | |
| 1.00E+02 | | 33.06 | 100 | | | |
| 1.00E+02 | | 31.78 | 100 | | | |
| 1.00E+01 | | 39.91 | 10 | | | |
| 1.00E+01 | | 40 | 0 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1552159Serprotease

| | Fold Change in Disease |
|---|---|
| | Population Relative to |
| Disease tissues | Normal |
| colon tumor | −1.92 |
| colon tumor | 1.21 |
| colon tumor | 1.24 |
| colon tumor | −1.91 |
| lung tumor | −37.40 |
| lung tumor | 3.09 |
| lung tumor | −3.78 |
| lung tumor | 3.59 |
| breast tumor | 3.76 |
| breast tumor | 1.72 |
| breast tumor | 2.66 |
| breast tumor | −16.24 |
| brain stage 5 ALZ | −1.04 |
| brain stage 5 ALZ | 3.05 |
| brain stage 5 ALZ | 2.38 |
| brain stage 5 ALZ | 3.04 |
| lung 24 COPD | −1.34 |
| lung 28 COPD | −3.68 |
| lung 23 COPD | −2.52 |
| asthmatic lung | −3.56 |
| asthmatic lung | 9.85 |
| asthmatic lung | 8.18 |
| asthmatic lung | 4.02 |
| endo VEGF | 1.55 |
| endo bFGF | 1.14 |
| heart (T-1) ischemic | 1.14 |
| heart (T-14) non-obstructive DCM | 1.49 |
| heart (T-3399) DCM | 1.27 |
| BM stim | −3.40 |
| osteo dif | 9.29 |
| OA Cartilage (pool) | −3.60 |
| PBL HIV IIIB | −23.72 |
| MRC5 HSV strain F | −4.44 |
| W12 cells | −8.73 |

Gene Name sbg1551159thymosinb4

High overall expression. This gene is expressed fairly ubiquitously in the normal samples with the highest levels of expression seen in the whole brain, endometrium, myometrium, rectum, spleen, uterus, and thymus. This gene is also expressed fairly ubiquitously in the disease samples with the highest levels of expression seen in the uninfected and infected PBLs. Upregulation in 2 of 4 colon tumor samples and 1 of 4 breast tumor samples is sufficient to claim that this gene may be involved in cancers of the colon and breast. Upregulation in 1 of 4 asthmatic lung samples with corroborating high expression in all of the inflammatory immune cells indicates a possible involvement in asthma Upregulation in 3 of 3 disease heart samples implies an involvement in cardiovascular diseases such as non-obstructive and obstructive DCM and ischemia. Downregulation in the HSV-infected MRC5 cells suggests that this gene may be a host factor in HSV. High expression in the RA and OA synovium samples, the OA bone samples, and the chondrocytes with corroborating high expression in all of the inflammatory immune cells implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1551159- | (sample | (sample | (sample | GOI | rRNA | rRNA | total |
| thymosinb4 | 1 and 2) | 1) | 2) | Copies | (ng) | (ng) | RNA |
| Subcutaneous | 21.19, 21.24 | 4954.06 | 4785.48 | 4869.77 | 3.06 | 16.34 | 79571.41 |
| Adipocytes Zenbio | | | | | | | |
| Subcutaneous Adipose | 25.39, 22.19 | 315.45 | 2561.93 | 1438.69 | 0.96 | 52.36 | 75324.08 |
| Zenbio | | | | | | | |
| Adrenal Gland Clontech | 26.25, 24.62 | 180.52 | 522.51 | 351.52 | 0.61 | 81.97 | 28812.70 |
| Whole Brain Clontech | 17.83, 17.65 | 44488.84 | 50016.56 | 47252.70 | 7.24 | 6.91 | 326330.80 |
| Fetal Brain Clontech | 25.1, 25.27 | 381.07 | 343.03 | 362.05 | 0.48 | 103.95 | 37635.14 |
| Cerebellum Clontech | 23.91, 23.79 | 830.52 | 902.3 | 866.41 | 2.17 | 23.04 | 19963.36 |
| Cervix | 22.08, 21.63 | 2754.7 | 3698.84 | 3226.77 | 2.42 | 20.66 | 66668.80 |
| Colon | 21.03, 21.04 | 5474.84 | 5453.74 | 5464.29 | 2.71 | 18.45 | 100817.16 |
| Endometrium | 21.96, 21.86 | 2977.38 | 3184.04 | 3080.71 | 0.73 | 68.21 | 210143.93 |
| Esophagus | 23.64, 23.34 | 996.91 | 1210.04 | 1103.48 | 1.37 | 36.50 | 40272.81 |
| Heart Clontech | 26.02, 24.86 | 209.22 | 447.18 | 328.20 | 1.32 | 37.88 | 12431.82 |
| Hypothalamus | 27.41, 27.27 | 84.36 | 92.31 | 88.34 | 0.32 | 155.28 | 13716.61 |
| Ileum | 21.97, 21.54 | 2968.68 | 3928.49 | 3448.59 | 2.58 | 19.38 | 66833.04 |
| Jejunum | 20.44, 20.44 | 8089.14 | 8083.85 | 8086.50 | 6.60 | 7.58 | 61261.33 |
| Kidney | 23.14, 22.11 | 1375.78 | 2708.16 | 2041.97 | 2.12 | 23.58 | 48159.67 |
| Liver | 24.97, 24.79 | 416.5 | 467.07 | 441.79 | 1.50 | 33.33 | 14726.17 |
| Fetal Liver Clontech | 19.45, 19.34 | 15432.97 | 16557.73 | 15995.35 | 10.40 | 4.81 | 76900.72 |
| Lung | 21.26, 21.5 | 4731.17 | 4046.45 | 4388.81 | 2.57 | 19.46 | 85385.41 |
| Mammary Gland | 19.29, 19.26 | 17125.19 | 17464.02 | 17294.61 | 13.00 | 3.85 | 66517.71 |
| Clontech | | | | | | | |
| Myometrium | 20.82, 20.28 | 6308.04 | 8972.5 | 7640.27 | 2.34 | 21.37 | 163253.63 |
| Omentum | 20.59, 20.26 | 7310.61 | 9084.23 | 8197.42 | 3.94 | 12.69 | 104028.17 |
| Ovary | 21.51, 21.22 | 4019.4 | 4853.94 | 4436.67 | 4.34 | 11.52 | 51113.71 |
| Pancreas | 26.01, 26.25 | 210.39 | 179.95 | 195.17 | 0.81 | 61.80 | 12062.42 |
| Head of Pancreas | 26.2, 25.66 | 185.8 | 265.23 | 225.52 | 1.57 | 31.85 | 7182.01 |
| Parotid Gland | 22.42, 21.99 | 2213.28 | 2931.58 | 2572.43 | 5.48 | 9.12 | 23471.08 |
| Placenta Clontech | 22.43, 22.48 | 2194.88 | 2126.18 | 2160.53 | 5.26 | 9.51 | 20537.36 |
| Prostate | 22.82, 22.96 | 1695.79 | 1547.57 | 1621.68 | 3.00 | 16.67 | 27028.00 |
| Rectum | 22.21, 22.05 | 2541.48 | 2810.64 | 2676.06 | 1.23 | 40.65 | 108782.93 |
| Salivary Gland Clontech | 22.03, 21.67 | 2852.39 | 3620.4 | 3236.40 | 7.31 | 6.84 | 22136.76 |
| Skeletal Muscle | 27.39, 27.13 | 85.16 | 100.96 | 93.06 | 1.26 | 39.68 | 3692.86 |
| Clontech | | | | | | | |
| Skin | 24.27, 23.9 | 656.73 | 838.66 | 747.70 | 1.21 | 41.32 | 30896.49 |
| Small Intestin Clontech | 26.86, 26.49 | 121.18 | 154.25 | 137.72 | 0.98 | 51.07 | 7033.45 |
| Spleen | 19.49, 19.43 | 15003.18 | 15611.73 | 15307.46 | 4.92 | 10.16 | 155563.57 |
| Stomach | 22.35, 21.82 | 2318.26 | 3278.55 | 2798.41 | 2.73 | 18.32 | 51252.84 |
| Testis Clontech | 28.06, 27.47 | 54.99 | 81.19 | 68.09 | 0.57 | 87.87 | 5983.30 |
| Thymus Clontech | 17.45, 17.18 | 57052.5 | 68169.71 | 62611.11 | 9.89 | 5.06 | 316537.44 |
| Thyroid | 23.74, 23.27 | 931.95 | 1264.88 | 1098.42 | 2.77 | 18.05 | 19826.99 |
| Trachea Clontech | 21.56, 21.25 | 3883.73 | 4744.74 | 4314.24 | 9.71 | 5.15 | 22215.42 |
| Urinary Bladder | 20.54, 20.19 | 7555.88 | 9539.87 | 8547.88 | 5.47 | 9.14 | 78134.14 |
| Uterus | 19.53, 19.13 | 14625.16 | 19031.66 | 16828.41 | 5.34 | 9.36 | 157569.38 |
| genomic | 23.18 | 1347.95 | | | | | |
| b-actin | 26.19 | 186.76 | | | | | |
| 1.00E+05 | 17.13 | 100000 | | | | | |
| 1.00E+05 | 17.01 | 100000 | | | | | |
| 1.00E+04 | 19.78 | 10000 | | | | | |
| 1.00E+04 | 19.89 | 10000 | | | | | |
| 1.00E+03 | 23.33 | 1000 | | | | | |
| 1.00E+03 | 23.21 | 1000 | | | | | |
| 1.00E+02 | 27.56 | 100 | | | | | |
| 1.00E+02 | 27.4 | 100 | | | | | |
| 1.00E+01 | 29.22 | 10 | | | | | |
| 1.00E+01 | 30.45 | 10 | | | | | |
| 1.00E+00 | 35.35 | 1 | | | | | |
| 1.00E+00 | 34.35 | 1 | | | | | |
| NTC | 40 | 0 | | | | | |
| NTC | 34.73 | −1 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1551159- | (GSK | | GOI | total | | Disease |
| thymosinb4 | identifier) | Ct | copies | RNA | Sample | Population |
| colon normal GW98-167 | 21941 | 16.75 | 198628.18 | 397256.36 | colon normal | |
| colon tumor GW98-166 | 21940 | 15.41 | 792265.93 | 1584531.87 | colon tumor | 3.99 |
| colon normal GW98-178 | 22080 | 20.06 | 9968.38 | 19936.77 | colon normal | |
| colon tumor GW98-177 | 22060 | 16.81 | 187186.80 | 374373.59 | colon tumor | 18.78 |
| colon normal GW98-561 | 23514 | 17.67 | 81798.86 | 163597.71 | colon normal | |
| colon tumor GW98-560 | 23513 | 17.23 | 124293.97 | 248587.94 | colon tumor | 1.52 |
| colon normal GW98-894 | 24691 | 16.49 | 257495.16 | 514990.32 | colon normal | |
| colon tumor GW98-893 | 24690 | 15.72 | 569314.61 | 1138629.22 | colon tumor | 2.21 |
| lung normal GW98-3 | 20742 | 16.06 | 399175.12 | 798350.24 | lung normal | |
| lung tumor GW98-2 | 20741 | 18.57 | 35873.21 | 71746.43 | lung tumor | −11.13 |
| lung normal GW97-179 | 20677 | 16.42 | 276326.62 | 552653.24 | lung normal | |
| lung tumor GW97-178 | 20676 | 16.05 | 403321.76 | 806643.52 | lung tumor | 1.46 |
| lung normal GW98-165 | 21922 | 16.23 | 335188.32 | 670376.65 | lung normal | |
| lung tumor GW98-164 | 21921 | 15.81 | 517863.70 | 1035727.40 | lung tumor | 1.54 |
| lung normal GW98-282 | 22584 | 17.35 | 110774.20 | 221548.39 | lung normal | |
| lung tumor GW98-281 | 22583 | 16.81 | 187186.80 | 374373.59 | lung tumor | 1.69 |
| breast normal GW00-392 | 28750 | 18.05 | 57468.10 | 57468.10 | breast normal | |
| breast tumor GW00-391 | 28746 | 18.1 | 54890.07 | 109780.14 | breast tumor | 1.91 |
| breast normal GW00-413 | 28798 | 17.68 | 81034.58 | 81034.58 | breast normal | |
| breast tumor GW00-412 | 28797 | 17.16 | 132979.44 | 265958.88 | breast tumor | 3.28 |
| breast normal GW00- | 27592-95 | 18.28 | 46578.77 | 46578.77 | breast normal | |
| 235:238 | | | | | | |
| breast tumor GW00- | 27588-91 | 17.38 | 107643.99 | 107643.99 | breast tumor | 2.31 |
| 231:234 | | | | | | |
| breast normal GW98-621 | 23656 | 17.7 | 79528.66 | 159057.32 | breast normal | |
| breast tumor GW98-620 | 23655 | 15.92 | 461587.39 | 923174.78 | breast tumor | 5.80 |
| brain normal BB99-542 | 25507 | 16.61 | 228308.17 | 456616.33 | brain normal | |
| brain normal BB99-406 | 25509 | 17.03 | 150862.61 | 301725.22 | brain normal | |
| brain normal BB99-904 | 25546 | 17.06 | 146520.74 | 293041.47 | brain normal | |
| brain stage 5 ALZ BB99- | 25502 | 18.71 | 31670.09 | 63340.18 | brain stage 5 | −5.53 |
| 874 | | | | | ALZ | |
| brain stage 5 ALZ BB99- | 25503 | 17.06 | 146520.74 | 293041.47 | brain stage 5 | −1.20 |
| 887 | | | | | ALZ | |
| brain stage 5 ALZ BB99- | 25504 | 18.11 | 54389.34 | 108778.68 | brain stage 5 | −3.22 |
| 862 | | | | | ALZ | |
| brain stage 5 ALZ BB99- | 25542 | 17.26 | 120757.64 | 241515.28 | brain stage 5 | −1.45 |
| 927 | | | | | ALZ | |
| CT lung | normal | 16.86 | 178186.17 | 356372.33 | CT lung Nml | |
| lung 26 | normal | 18.57 | 35873.21 | | lung 26 Nml | |
| lung 27 | normal | 18.73 | 31113.64 | 31113.64 | lung 27 Nml | |
| lung 24 | COPD | 19.35 | 18125.41 | 18125.41 | lung 24 COPD | −8.20 |
| lung 28 | COPD | 18.58 | 35554.21 | 35554.21 | lung 28 COPD | −4.18 |
| lung 23 | COPD | 19.47 | 16358.32 | 16358.32 | lung 23 COPD | −9.09 |
| lung 25 | normal | 18.03 | 58534.98 | 58534.98 | lung 25 Nml | |
| asthmatic lung ODO3112 | 29321 | 17.08 | 143699.90 | 143699.90 | asthmatic lung | −1.03 |
| asthmatic lung ODO3433 | 29323 | 16.87 | 176441.75 | 352883.50 | asthmatic lung | 2.37 |
| asthmatic lung ODO3397 | 29322 | 15.84 | 501828.37 | 1003656.73 | asthmatic lung | 6.75 |
| asthmatic lung ODO4928 | 29325 | 16.58 | 235259.88 | 470519.77 | asthmatic lung | 3.16 |
| endo cells | control | 17.3 | 116207.60 | 116207.60 | endo cells | |
| endo VEGF | | 17.49 | 96945.41 | 96945.41 | endo VEGF | −1.20 |
| endo bFGF | | 18.04 | 57998.94 | 57998.94 | endo bFGF | −2.00 |
| heart Clontech | normal | 19.29 | 19083.85 | 38167.71 | heart | |
| heart (T-1) ischemic | 29417 | 17.76 | 75186.28 | 150372.55 | heart (T-1) | 3.94 |
| | | | | | ischemic | |
| heart (T-14) non- | 29422 | 17.85 | 69137.71 | 138275.42 | heart (T-14) non- | 3.62 |
| obstructive DCM | | | | | obstructive DCM | |
| heart (T-3399) DCM | 29426 | 17.53 | 93339.68 | 186679.35 | heart (T-3399) | 4.89 |
| | | | | | DCM | |
| adenoid GW99-269 | 26162 | 16.25 | 328408.78 | 656817.56 | adenoid | |
| tonsil GW98-280 | 22582 | 15.72 | 569314.61 | 1138629.22 | tonsil | |
| T cells PC00314 | 28453 | 16.39 | 284839.38 | 569678.76 | T cells | |
| PBMNC | | 18.07 | 56421.81 | 56421.81 | PBMNC | |
| monocyte | | 17.81 | 71759.68 | 143519.37 | monocyte | |
| B cells PC00665 | 28455 | 17.27 | 119602.70 | 239205.39 | B cells | |
| dendritic cells | 28441 | 15.44 | 767107.95 | 1534215.89 | dendritic cells | |
| neutrophils | 28440 | 15.1 | 1109957.39 | 1109957.39 | neutrophils | |
| eosinophils | 28446 | 16.12 | 375225.96 | 750451.92 | eosinophils | |
| BM unstim | | 17.12 | 138229.52 | 138229.52 | BM unstim | |
| BM stim | treated | 16.1 | 383035.13 | 383035.13 | BM stim | 2.77 |
| osteo dif | treated | 16.95 | 163121.51 | 163121.51 | osteo dif | 2.79 |
| osteo undif | | 18.03 | 58534.98 | 58534.98 | osteo undif | |
| chondrocytes | | 16.89 | 173007.01 | 432517.52 | chondrocytes | |
| OA Synovium IP12/01 | 29462 | 16.23 | 335188.32 | 335188.32 | OA Synovium | |
| OA Synovium NP10/01 | 29461 | 17.62 | 85736.55 | 171473.11 | OA Synovium | |
| OA Synovium NP57/00 | 28464 | 16.24 | 331780.19 | 663560.38 | OA Synovium | |
| RA Synovium | 28466 | 15.7 | 581468.02 | 1162936.04 | RA Synovium | |
| NP03/01 | | | | | | |
| RA Synovium | 28467 | 16.3 | 312088.06 | 624176.11 | RA Synovium | |
| NP71/00 | | | | | | |
| RA Synovium | 28475 | 16.02 | 416038.02 | 832076.03 | RA Synovium | |
| NP45/00 | | | | | | |
| OA bone (biobank) | 29217 | 17.28 | 118459.46 | 118459.46 | OA bone | |
| | | | | | (biobank) | |
| OA bone Sample 1 | J. Emory | 16.52 | 249845.54 | 499691.09 | OA bone | |
| OA bone Sample 2 | J. Emory | 16.65 | 219376.15 | 438752.31 | OA bone | |
| Cartilage (pool) | Normal | 17.02 | 152340.05 | 304680.10 | Nml Cartilage | |
| | | | | | (pool) | |
| Cartilage (pool) | OA | 17.55 | 91590.38 | 183180.77 | OA Cartilage | −1.66 |
| | | | | | (pool) | |
| PBL unifected | 28441 | 14.25 | 2902645.67 | 5805291.34 | PBL unifected | |
| PBL HIV IIIB | 28442 | 15.12 | 1085846.74 | 2171693.49 | PBL HIV IIIB | −2.67 |
| MRC5 uninfected (100%) | 29158 | 16.31 | 308928.36 | 617856.71 | MRC5 | |
| | | | | | uninfected | |
| | | | | | (100%) | |
| MRC5 HSV strain F | 29178 | 22.02 | 2122.64 | 4245.29 | MRC5 HSV | −145.54 |
| | | | | | strain F | |
| W12 cells | 29179 | 17.46 | 99746.51 | 199493.02 | W12 cells | −1.28 |
| Keratinocytes | 29180 | 17.2 | 127940.29 | 255880.58 | Keratinocytes | |
| B-actin control | | 25.54 | 181.26 | | | |
| genomic | | 23 | 1030.66 | Copy number was calculated from the | | |
| | | | | standard curve run on the normal plate | | |
| 1.00E+05 | | | | | | |
| 1.00E+05 | | | | | | |
| 1.00E+04 | | | | | | |
| 1.00E+04 | | | | | | |
| 1.00E+03 | | | | | | |
| 1.00E+03 | | | | | | |
| 1.00E+02 | | | | | | |
| 1.00E+02 | | | | | | |
| 1.00E+01 | | | | | | |
| 1.00E+01 | | | | | | |
| 1.00E+00 | | | | | | |
| 1.00E+00 | | | | | | |
| NTC | | 40 | 0.11 | | | |

*lung 26 Normal has been omitted due to multiple amplification failures from that sample

Gene Name sbg1551159thymosinb4

| | Fold Change in Disease |
|---|---|
| | Population Relative to |
| Disease tissues | Normal |
| colon tumor | 3.99 |
| colon tumor | 18.78 |
| colon tumor | 1.52 |
| colon tumor | 2.21 |
| lung tumor | −11.13 |
| lung tumor | 1.46 |
| lung tumor | 1.54 |
| lung tumor | 1.69 |
| breast tumor | 1.91 |
| breast tumor | 3.28 |
| breast tumor | 2.31 |
| breast tumor | 5.80 |
| brain stage 5 ALZ | −5.53 |
| brain stage 5 ALZ | −1.20 |
| brain stage 5 ALZ | −3.22 |
| brain stage 5 ALZ | −1.45 |
| lung 24 COPD | −8.20 |
| lung 28 COPD | −4.18 |
| lung 23 COPD | −9.09 |
| asthmatic lung | −1.03 |
| asthmatic lung | 2.37 |
| asthmatic lung | 6.75 |
| asthmatic lung | 3.16 |
| endo VEGF | −1.20 |
| endo bFGF | −2.00 |
| heart (T-1) ischemic | 3.94 |
| heart (T-14) non-obstructive DCM | 3.62 |
| heart (T-3399) DCM | 4.89 |
| BM stim | 2.77 |
| osteo dif | 2.79 |
| OA Cartilage (pool) | −1.66 |
| PBL HIV IIIB | −2.67 |
| MRC5 HSV strain F | −145.54 |
| W12 cells | −1.28 |

Gene Name SBh1686954.SOCS

Moderate to high overall expression. The highest disease expression is seen in the whole brain, fetal liver, and thymus. The expression of this gene in the disease samples is fairly ubiquitous with the highest expression seen in the uninfected PBLs. Upregulation in 1 of 4 breast tumor samples implicates this gene in cancer of the breast Upregulation in 2 of 4 asthmatic lung samples with corroborating high expression in the eosinophils and neutrophils indicates a possible involvement in asthma. Upregulation in 3 of 3 disease heart samples implies an involvement in cardiovascular diseases such as non-obstructive and obstructive DCM and ischemia. Upregulated in the stimulated bone marrow. Downregulation in the HSV-infected MRC5 cells suggests that this gene may be a host factor in HSV. High expression in the RA and OA synovium samples, the OA bone samples, and the chondrocytes with corroborating high expression in T cells, B cells, dendritic cells, eosinophils, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| SBh1686954.SOCS | 1 and 2) | 1) | 2) | Copies | (ng) | (ng) | RNA |
| Subcutaneous | 32.3, 32.23 | 71.17 | 73.85 | 72.51 | 3.06 | 16.34 | 1184.80 |
| Adipocyt s Zenbio | | | | | | | |
| Subcutaneous | 40, 35.35 | 0 | 11.59 | 5.80 | 0.96 | 52.36 | 303.40 |
| Adipose Zenbio | | | | | | | |
| Adrenal Gland | 36.03, 40 | 7.75 | 0 | 3.88 | 0.61 | 81.97 | 317.62 |
| Clontech | | | | | | | |
| Whole Brain Clontech | 23.81, 24.1 | 10980.32 | 9261.99 | 10121.16 | 7.24 | 6.91 | 69897.48 |
| Fetal Brain Clontech | 36.67, 36.09 | 5.3 | 7.46 | 6.38 | 0.48 | 103.95 | 663.20 |
| Cerebellum Clontech | 32.53, 33.22 | 62.11 | 41.03 | 51.57 | 2.17 | 23.04 | 1188.25 |
| Cervix | 33.29, 33.86 | 39.34 | 28.13 | 33.74 | 2.42 | 20.66 | 697.00 |
| Colon | 32.35, 32.44 | 68.76 | 65.19 | 66.98 | 2.71 | 18.45 | 1235.70 |
| Endometrium | 34.23, 34.76 | 22.57 | 16.49 | 19.53 | 0.73 | 68.21 | 1332.20 |
| Esophagus | 33.87, 32.62 | 27.89 | 58.8 | 43.35 | 1.37 | 36.50 | 1581.93 |
| Heart Clontech | 33.96, 34.93 | 26.56 | 14.88 | 20.72 | 1.32 | 37.88 | 784.85 |
| Hypothalamus | 35.05, 40 | 13.88 | 0 | 6.94 | 0.32 | 155.28 | 1077.64 |
| Ileum | 33.1, 32.27 | 44.29 | 72.1 | 58.20 | 2.58 | 19.38 | 1127.81 |
| Jejunum | 30.33, 30.78 | 228.25 | 174.81 | 201.53 | 6.60 | 7.58 | 1526.74 |
| Kidney | 34.57, 32.85 | 18.49 | 51.19 | 34.84 | 2.12 | 23.58 | 821.70 |
| Liver | 34.31, 35.6 | 21.5 | 9.98 | 15.74 | 1.50 | 33.33 | 524.67 |
| Fetal Liver Clontech | 27.29, 27.16 | 1395.02 | 1500.44 | 1447.73 | 10.40 | 4.81 | 6960.24 |
| Lung | 32.91, 32.27 | 49.38 | 72.19 | 60.79 | 2.57 | 19.46 | 1182.59 |
| Mammary Gland | 29.07, 28.86 | 483.61 | 546.94 | 515.28 | 13.00 | 3.85 | 1981.83 |
| Clontech | | | | | | | |
| Myometrium | 32.22, 31.71 | 74.6 | 100.64 | 87.62 | 2.34 | 21.37 | 1872.22 |
| Omentum | 32.63, 32.61 | 58.22 | 58.94 | 58.58 | 3.94 | 12.69 | 743.40 |
| Ovary | 31.47, 32.28 | 116.33 | 71.94 | 94.14 | 4.34 | 11.52 | 1084.50 |
| Pancreas | 36.97, 34.24 | 4.43 | 22.51 | 13.47 | 0.81 | 61.80 | 832.51 |
| Head of Pancreas | 35.86, 35.9 | 8.58 | 8.37 | 8.48 | 1.57 | 31.85 | 269.90 |
| Parotid Gland | 33.32, 32.02 | 38.8 | 83.82 | 61.31 | 5.48 | 9.12 | 559.40 |
| Placenta Clontech | 32.27, 31.3 | 72.4 | 128.39 | 100.40 | 5.26 | 9.51 | 954.33 |
| Prostate | 32.41, 33.27 | 66.44 | 40.02 | 53.23 | 3.00 | 16.67 | 887.17 |
| Rectum | 32.22, 31.7 | 74.56 | 101.26 | 87.91 | 1.23 | 40.65 | 3573.58 |
| Salivary Gland | 31.41, 31.63 | 120.65 | 105.6 | 113.13 | 7.31 | 6.84 | 773.77 |
| Clontech | | | | | | | |
| Skeletal Muscle | 36.03, 40 | 7.74 | 0.15 | 3.95 | 1.26 | 39.68 | 156.55 |
| Clontech | | | | | | | |
| Skin | 34.93, 35.01 | 14.9 | 14.2 | 14.55 | 1.21 | 41.32 | 601.24 |
| Small Intestine | 40, 39.62 | 3 | 0.92 | 1.96 | 0.98 | 51.07 | 100.10 |
| Clontech | | | | | | | |
| Spleen | 31.69, 31.36 | 102.03 | 124.35 | 113.19 | 4.92 | 10.16 | 1150.30 |
| Stomach | 33.11, 31.62 | 43.83 | 106.19 | 75.01 | 2.73 | 18.32 | 1373.81 |
| Testis Clontech | 33.57, 34.29 | 33.4 | 21.76 | 27.58 | 0.57 | 87.87 | 2423.55 |
| Thymus Clontech | 25.01, 24.89 | 5395.44 | 5795.91 | 5595.68 | 9.89 | 5.06 | 28289.56 |
| Thyroid | 33.46, 33.09 | 35.61 | 44.36 | 39.99 | 2.77 | 18.05 | 721.75 |
| Trachea Clontech | 31.52, 31.18 | 112.93 | 138.06 | 125.50 | 9.71 | 5.15 | 646.22 |
| Urinary Bladder | 30.13, 29.48 | 258.39 | 378.54 | 318.47 | 5.47 | 9.14 | 2911.01 |
| Uterus | 31.08, 31.12 | 147.02 | 143.19 | 145.11 | 5.34 | 9.36 | 1358.66 |
| genomic | 27.15 | 1510.74 | | | | | |
| b-actin | 28.33 | 750.86 | | | | | |
| 1.00E+05 | 20.48 | 100000 | | | | | |
| 1.00E+05 | 20.41 | 100000 | | | | | |
| 1.00E+04 | 23.74 | 10000 | | | | | |
| 1.00E+04 | 23.67 | 10000 | | | | | |
| 1.00E+03 | 27.33 | 1000 | | | | | |
| 1.00E+03 | 27.12 | 1000 | | | | | |
| 1.00E+02 | 33.04 | 100 | | | | | |
| 1.00E+02 | 31.66 | 100 | | | | | |
| 1.00E+01 | 35.21 | 10 | | | | | |
| 1.00E+01 | 35.81 | 10 | | | | | |
| 1.00E+00 | 40 | 0 | | | | | |
| 1.00E+00 | 40 | 0 | | | | | |
| NTC | 40 | −1 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| SBh1686954.SOCS | identifier) | Ct | copies | RNA | Sample | Population |
| colon normal GW98-167 | 21941 | 25.21 | 5620.96 | 11241.92 | colon normal | |
| colon tumor GW98-166 | 21940 | 25.47 | 4871.05 | 9742.10 | colon tumor | −1.15 |
| colon normal GW98-178 | 22080 | 27.8 | 1370.8 | 2741.60 | colon normal | |
| colon tumor GW98-177 | 22060 | 25.49 | 4833.72 | 9667.44 | colon tumor | 3.53 |
| colon normal GW98-561 | 23514 | 26.34 | 3033.87 | 6067.74 | colon normal | |
| colon tumor GW98-560 | 23513 | 26.18 | 3313.57 | 6627.14 | colon tumor | 1.09 |
| colon normal GW98-894 | 24691 | 24.96 | 6438.53 | 12877.06 | colon normal | |
| colon tumor GW98-893 | 24690 | 25.12 | 5911.83 | 11823.66 | colon tumor | −1.09 |
| lung normal GW98-3 | 20742 | 25.36 | 5173.46 | 10346.92 | lung normal | |
| lung tumor GW98-2 | 20741 | 26.06 | 3540 | 7080.00 | lung tumor | −1.46 |
| lung normal GW97-179 | 20677 | 24.02 | 10757.4 | 21514.80 | lung normal | |
| lung tumor GW97-178 | 20676 | 25.27 | 5443.34 | 10886.68 | lung tumor | −1.98 |
| lung normal GW98-165 | 21922 | 25.16 | 5771.16 | 11542.32 | lung normal | |
| lung tumor GW98-164 | 21921 | 25.38 | 5122.16 | 10244.32 | lung tumor | −1.13 |
| lung normal GW98-282 | 22584 | 25.2 | 5659.86 | 11319.72 | lung normal | |
| lung tumor GW98-281 | 22583 | 26.67 | 2543.14 | 5086.28 | lung tumor | −2.23 |
| breast normal GW00-392 | 28750 | 26.49 | 2804.16 | 2804.16 | breast normal | |
| breast tumor GW00-391 | 28746 | 26.22 | 3249.93 | 6499.86 | breast tumor | 2.32 |
| breast normal GW00-413 | 28798 | 28.07 | 1183.39 | 1183.39 | breast normal | |
| breast tumor GW00-412 | 28797 | 25.76 | 4176.56 | 8353.12 | breast tumor | 7.06 |
| breast normal GW00- | 27592-95 | 28.35 | 1014.62 | 1014.62 | breast normal | |
| 235:238 | | | | | | |
| breast tumor GW00- | 27588-91 | 26.06 | 3531.95 | 3531.95 | breast tumor | 3.48 |
| 231:234 | | | | | | |
| breast normal GW98-621 | 23656 | 25.4 | 5077.73 | 10155.46 | breast normal | |
| breast tumor GW98-620 | 23655 | 25.87 | 3922.14 | 7844.28 | breast tumor | −1.29 |
| brain normal BB99-542 | 25507 | 25.15 | 5812.78 | 11625.56 | brain normal | |
| brain normal BB99-406 | 25509 | 25.82 | 4043.66 | 8087.32 | brain normal | |
| brain normal BB99-904 | 25546 | 26.05 | 3549.95 | 7099.90 | brain normal | |
| brain stage 5 | 25502 | 26.16 | 3351.06 | 6702.12 | brain stage | −1.33 |
| ALZ BB99-874 | | | | | 5 ALZ | |
| brain stage 5 | 25503 | 24.09 | 10322.56 | 20645.12 | brain stage | 2.31 |
| ALZ BB99-887 | | | | | 5 ALZ | |
| brain stage 5 | 25504 | 24.46 | 8438.18 | 16876.36 | brain stage | 1.89 |
| ALZ BB99-862 | | | | | 5 ALZ | |
| brain stage 5 | 25542 | 24.81 | 7008.05 | 14016.10 | brain stage | 1.57 |
| ALZ BB99-927 | | | | | 5 ALZ | |
| CT lung | normal | 27.44 | 1667.22 | 3334.44 | CT lung Nml | |
| lung 26 | normal | 30.89 | 254.94 | | lung 26 Nml | |
| lung 27 | normal | 31.43 | 189.67 | 189.67 | lung 27 Nml | |
| lung 24 | COPD | 31.8 | 155.1 | 155.10 | lung 24 COPD | −8.09 |
| lung 28 | COPD | 30.1 | 392.91 | 392.91 | lung 28 COPD | −3.19 |
| lung 23 | COPD | 31.25 | 209.08 | 209.08 | lung 23 COPD | −6.00 |
| lung 25 | normal | 31 | 240.09 | 240.09 | lung 25 Nml | |
| asthmatic lung ODO3112 | 29321 | 27.63 | 1504.34 | 1504.34 | asthmatic lung | 1.20 |
| asthmatic lung ODO3433 | 29323 | 26.88 | 2262.28 | 4524.56 | asthmatic lung | 3.61 |
| asthmatic lung ODO3397 | 29322 | 25.01 | 6274.02 | 12548.04 | asthmatic lung | 10.00 |
| asthmatic lung ODO4928 | 29325 | 26.38 | 2978.98 | 5957.96 | asthmatic lung | 4.75 |
| endo cells | control | 29.16 | 654.47 | 654.47 | endo cells | |
| endo VEGF | | 29.35 | 589.28 | 589.28 | endo VEGF | −1.11 |
| endo bFGF | | 29.29 | 608.94 | 608.94 | endo bFGF | −1.07 |
| heart Clontech | normal | 31.92 | 145.7 | 291.40 | heart | |
| heart (T-1) ischemic | 29417 | 25.47 | 4876.38 | 9752.76 | heart (T-1) | 33.47 |
| | | | | | ischemic | |
| heart (T-14) non- | 29422 | 25.27 | 5449.4 | 10898.80 | heart (T-14) non- | 37.40 |
| obstructive DCM | | | | | obstructive DCM | |
| heart (T-3399) DCM | 29426 | 24.97 | 6405.26 | 12810.52 | heart (T-3399) | 43.96 |
| | | | | | DCM | |
| adenoid GW99-269 | 26162 | 26.65 | 2571.85 | 5143.70 | adenoid | |
| tonsil GW98-280 | 22582 | 24.76 | 7176.4 | 14352.80 | tonsil | |
| T cells PC00314 | 28453 | 24.46 | 8477.94 | 16955.88 | T cells | |
| PBMNC | | 31.29 | 204.63 | 204.63 | PBMNC | |
| monocyte | | 32.64 | 98.31 | 196.62 | monocyte | |
| B cells PC00665 | 28455 | 25.27 | 5432.39 | 10864.78 | B cells | |
| dendritic cells | 28441 | 25.34 | 5235.78 | 10471.56 | dendritic cells | |
| neutrophils | 28440 | 26.4 | 2934.13 | 2934.13 | neutrophils | |
| eosinophils | 28446 | 27.63 | 1501.86 | 3003.72 | eosinophils | |
| BM unstim | | 31.26 | 208.54 | 208.54 | BM unstim | |
| BM stim | treated | 28.19 | 1107.88 | 1107.88 | BM stim | 5.31 |
| osteo dif | treated | 28.42 | 978.61 | 978.61 | osteo dif | 2.72 |
| osteo undif | | 30.26 | 359.85 | 359.85 | osteo undif | |
| chondrocytes | | 25.79 | 4101.89 | 10254.73 | chondrocytes | |
| OA Synovium IP12/01 | 29462 | 25.83 | 4018.19 | 4018.19 | OA Synovium | |
| OA Synovium NP10/01 | 29461 | 27.08 | 2028.25 | 4056.50 | OA Synovium | |
| OA Synovium NP57/00 | 28464 | 26.04 | 3586.44 | 7172.88 | OA Synovium | |
| RA Synovium | 28466 | 25.23 | 5575.78 | 11151.56 | RA Synovium | |
| NP03/01 | | | | | | |
| RA Synovium | 28467 | 25.5 | 4801.07 | 9602.14 | RA Synovium | |
| NP71/00 | | | | | | |
| RA Synovium | 28475 | 25.28 | 5411.66 | 10823.32 | RA Synovium | |
| NP45/00 | | | | | | |
| OA bone (biobank) | 29217 | 27.78 | 1384.45 | 1384.45 | OA bone (biobank) | |
| OA bone Sample 1 | J. Emory | 26.36 | 3001.78 | 6003.56 | OA bone | |
| OA bone Sample 2 | J. Emory | 26.33 | 3061.4 | 6122.80 | OA bone | |
| Cartilage (pool) | Normal | 26.08 | 3495.6 | 6991.20 | Nml Cartilage | |
| | | | | | (pool) | |
| Cartilage (pool) | OA | 27.28 | 1817.93 | 3635.86 | OA Cartilage | −1.92 |
| | | | | | (pool) | |
| PBL unifected | 28441 | 23.28 | 16055.84 | 32111.68 | PBL unifected | |
| PBL HIV IIIB | 28442 | 24.79 | 7050.65 | 14101.30 | PBL HIV IIIB | −2.28 |
| MRC5 uninfected | 29158 | 24.72 | 7336.23 | 14672.46 | MRC5 uninfected | |
| (100%) | | | | | (100%) | |
| MRC5 HSV strain F | 29178 | 29.64 | 502.36 | 1004.72 | MRC5 HSV strain F | −14.60 |
| W12 cells | 29179 | 25.13 | 5884.35 | 11768.70 | W12 cells | −1.36 |
| Keratinocytes | 29180 | 24.56 | 8015.6 | 16031.20 | Keratinocytes | |
| B-actin control | | 27.84 | 1345.71 | | | |
| genomic | | 27.48 | 1632.59 | | | |
| 1.00E+05 | | 20.4 | 100000 | | | |
| 1.00E+05 | | 20.45 | 100000 | | | |
| 1.00E+04 | | 24.08 | 10000 | | | |
| 1.00E+04 | | 24.06 | 10000 | | | |
| 1.00E+03 | | 27.51 | 1000 | | | |
| 1.00E+03 | | 27.75 | 1000 | | | |
| 1.00E+02 | | 32.65 | 100 | | | |
| 1.00E+02 | | 32.07 | 100 | | | |
| 1.00E+01 | | 39.7 | 10 | | | |
| 1.00E+01 | | 35.14 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name SBh1686954.SOCS

| | Fold Change in Disease |
|---|---|
| | Population Relative to |
| Disease tissues | Normal |
| colon tumor | −1.15 |
| colon tumor | 3.53 |
| colon tumor | 1.09 |
| colon tumor | −1.09 |
| lung tumor | −1.46 |
| lung tumor | −1.98 |
| lung tumor | −1.13 |
| lung tumor | −2.23 |
| breast tumor | 2.32 |
| breast tumor | 7.06 |
| breast tumor | 3.48 |
| breast tumor | −1.29 |
| brain stage 5 ALZ | −1.33 |
| brain stage 5 ALZ | 2.31 |
| brain stage 5 ALZ | 1.89 |
| brain stage 5 ALZ | 1.57 |
| lung 24 COPD | −8.09 |
| lung 28 COPD | −3.19 |
| lung 23 COPD | −6.00 |
| asthmatic lung | 1.20 |
| asthmatic lung | 3.61 |
| asthmatic lung | 10.00 |
| asthmatic lung | 4.75 |
| endo VEGF | −1.11 |
| endo bFGF | −1.07 |
| heart (T-1) ischemic | 33.47 |
| heart (T-14) non-obstructive DCM | 37.40 |
| heart (T-3399) DCM | 43.96 |
| BM stim | 5.31 |
| osteo dif | 2.72 |
| OA Cartilage (pool) | −1.92 |
| PBL HIV IIIB | −2.28 |
| MRC5 HSV strain F | −14.60 |
| W12 cells | −1.36 |

Gene Name sbg1548844slit

Moderate overall expression. The highest normal expression is seen in the whole brain, cerebellum, and thymus. The expression in the disease samples is highly specific to most of the immune cells including the adenoid, tonsil, T cells, B cells, dendritic cells, neutrophils, eosinophils, and the uninfected peripheral blood lymphocytes. Upregulation in 3 of 4 asthmatic lung samples with corroborating high expression in the immune cells indicates a possible involvement in asthma. Upregulation in the HSV-infected MRC5 cells and the W12 cells suggests that this gene may be a host factor in HSV and HPV. High expression in the RA synovium samples with corroborating high immune cell expression implicates this gene in rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| Sample | Ct | copies | copies | Average | 18 S | 18 S | 50 ng |
| sbg1548844- | (sample | (sample | (sample | GOI | rRNA | rRNA | total |
| slit | 1 and 2) | 1) | 2) | Copies | (ng) | (ng) | RNA |

| | | | | | | | |
|---|---|---|---|---|---|---|---|
| Subcutaneous | 35.66, 35.22 | 13.12 | 17.14 | 15.13 | 3.06 | 16.34 | 247.22 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 1.15 | 0.58 | 0.61 | 81.97 | 47.13 |
| Whole Brain Clontech | 24.5, 24.47 | 11183.13 | 11425.02 | 11304.08 | 7.24 | 6.91 | 78066.82 |
| Fetal Brain Clontech | 40, 34.17 | 0 | 32.33 | 16.17 | 0.48 | 103.95 | 1680.35 |
| Cerebellum Clontech | 30.27, 30.05 | 341.45 | 390.51 | 365.98 | 2.17 | 23.04 | 8432.72 |
| Cervix | 37.28, 35.24 | 4.93 | 16.91 | 10.92 | 2.42 | 20.66 | 225.62 |
| Colon | 34.68, 34.59 | 23.72 | 25.1 | 24.41 | 2.71 | 18.45 | 450.37 |
| Endometrium | 35.92, 40 | 11.2 | 0 | 5.60 | 0.73 | 68.21 | 381.99 |
| Esophagus | 36.08, 34.63 | 10.14 | 24.48 | 17.31 | 1.37 | 36.50 | 631.75 |
| Heart Clontech | 40, 39.79 | 0 | 1.08 | 0.54 | 1.32 | 37.88 | 20.45 |
| Hypothalamus | 36.2, 35.94 | 9.48 | 11.06 | 10.27 | 0.32 | 155.28 | 1594.72 |
| Ileum | 35.06, 32.36 | 18.85 | 96.29 | 57.57 | 2.58 | 19.38 | 1115.70 |
| Jejunum | 32.51, 33.11 | 87.93 | 61.21 | 74.57 | 6.60 | 7.58 | 564.92 |
| Kidney | 40, 40 | 0 | 0 | 0.00 | 2.12 | 23.58 | 0.00 |
| Liver | 40, 34.67 | 0.58 | 23.92 | 12.25 | 1.50 | 33.33 | 408.33 |
| Fetal Liver Clontech | 33.24, 32.48 | 56.8 | 89.69 | 73.25 | 10.40 | 4.81 | 352.14 |
| Lung | 34.9, 34.3 | 20.72 | 29.76 | 25.24 | 2.57 | 19.46 | 491.05 |
| Mammary Gland | 33.77, 32.06 | 41.15 | 115.89 | 78.52 | 13.00 | 3.85 | 302.00 |
| Clontech | | | | | | | |
| Myometrium | 34.19, 32.29 | 31.86 | 100.92 | 66.39 | 2.34 | 21.37 | 1418.59 |
| Omentum | 36.99, 36.81 | 5.85 | 6.54 | 6.20 | 3.94 | 12.69 | 78.62 |
| Ovary | 36.31, 35.43 | 8.83 | 15.02 | 11.93 | 4.34 | 11.52 | 137.38 |
| Pancreas | 40, 40 | 0 | 0.48 | 0.24 | 0.81 | 61.80 | 14.83 |
| Head of Pancreas | 40, 40 | 0.66 | 0 | 0.33 | 1.57 | 31.85 | 10.51 |
| Parotid Gland | 34.63, 34.72 | 24.44 | 23.13 | 23.79 | 5.48 | 9.12 | 217.02 |
| Placenta Clontech | 35.8, 34.44 | 12.06 | 27.49 | 19.78 | 5.26 | 9.51 | 187.98 |
| Prostate | 40, 40 | 0 | 0 | 0.00 | 3.00 | 16.67 | 0.00 |
| Rectum | 36.18, 35.32 | 9.59 | 16.14 | 12.87 | 1.23 | 40.65 | 522.97 |
| Salivary Gland Clontech | 33.94, 33.93 | 37.04 | 37.34 | 37.19 | 7.31 | 6.84 | 254.38 |
| Skeletal Muscle | 40, 40 | 0 | 0 | 0.00 | 1.26 | 39.68 | 0.00 |
| Clontech | | | | | | | |
| Skin | 40, 36.59 | 0.56 | 7.48 | 4.02 | 1.21 | 41.32 | 166.12 |
| Small Intestine Clontech | 38.3, 38.52 | 2.65 | 2.33 | 2.49 | 0.98 | 51.07 | 127.17 |
| Spleen | 32.12, 33.2 | 111.43 | 57.88 | 84.66 | 4.92 | 10.16 | 860.32 |
| Stomach | 36.39, 36.57 | 8.44 | 7.56 | 8.00 | 2.73 | 18.32 | 146.52 |
| Testis Clontech | 36.46, 35.95 | 8.09 | 10.97 | 9.53 | 0.57 | 87.87 | 837.43 |
| Thymus Clontech | 29.65, 29.31 | 496.27 | 611.35 | 553.81 | 9.89 | 5.06 | 2799.85 |
| Thyroid | 35.46, 40 | 14.78 | 0.58 | 7.68 | 2.77 | 18.05 | 138.63 |
| Trachea Clontech | 33.19, 33.11 | 58.51 | 61.4 | 59.96 | 9.71 | 5.15 | 308.73 |
| Urinary Bladder | 34.52, 40 | 26.07 | 0.49 | 13.28 | 5.47 | 9.14 | 121.39 |
| Uterus | 35.15, 34.22 | 17.85 | 31.25 | 24.55 | 5.34 | 9.36 | 229.87 |
| genomic | 28.36 | 1082.56 | | | | | |
| b-actin | 27.98 | 1361.09 | | | | | |
| 1.00E+05 | 20.83 | 100000 | | | | | |
| 1.00E+05 | 21.28 | 100000 | | | | | |
| 1.00E+04 | 24.41 | 10000 | | | | | |
| 1.00E+04 | 24.5 | 10000 | | | | | |
| 1.00E+03 | 28.3 | 1000 | | | | | |
| 1.00E+03 | 28.59 | 1000 | | | | | |
| 1.00E+02 | 32.78 | 100 | | | | | |
| 1.00E+02 | 32.02 | 100 | | | | | |
| 1.00E+01 | 36.15 | 10 | | | | | |
| 1.00E+01 | 36.09 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1548844- | (GSK | | GOI | total | | Disease |
| slit | identifier) | Ct | copies | RNA | Sample | Population |
| colon normal GW98-167 | 21941 | 28.08 | 1161.39 | 2322.78 | colon normal | |
| colon tumor GW98-166 | 21940 | 28.55 | 883.23 | 1766.46 | colon tumor | −1.31 |
| colon normal GW98-178 | 22080 | 29.89 | 408.18 | 816.36 | colon normal | |
| colon tumor GW98-177 | 22060 | 31.89 | 129.04 | 258.08 | colon tumor | −3.16 |
| colon normal GW98-561 | 23514 | 28.25 | 1047.87 | 2095.74 | colon normal | |
| colon tumor GW98-560 | 23513 | 30.26 | 330.61 | 661.22 | colon tumor | −3.17 |
| colon normal GW98-894 | 24691 | 30.06 | 369 | 738.00 | colon normal | |
| colon tumor GW98-893 | 24690 | 30.38 | 307.17 | 614.34 | colon tumor | −1.20 |
| lung normal GW98-3 | 20742 | 28.22 | 1069.03 | 2138.06 | lung normal | |
| lung tumor GW98-2 | 20741 | 31.95 | 124.8 | 249.60 | lung tumor | −8.57 |
| lung normal GW97-179 | 20677 | 26.31 | 3214.71 | 6429.42 | lung normal | |
| lung tumor GW97-178 | 20676 | 28.73 | 797.3 | 1594.60 | lung tumor | −4.03 |
| lung normal GW98-165 | 21922 | 28.38 | 976.74 | 1953.48 | lung normal | |
| lung tumor GW98-164 | 21921 | 27.99 | 1219.61 | 2439.22 | lung tumor | 1.25 |
| lung normal GW98-282 | 22584 | 28.53 | 892.32 | 1784.64 | lung normal | |
| lung tumor GW98-281 | 22583 | 29.35 | 557.89 | 1115.78 | lung tumor | −1.60 |
| breast normal GW00-392 | 28750 | 29.91 | 403.21 | 403.21 | breast normal | |
| breast tumor GW00-391 | 28746 | 29.9 | 405.69 | 811.38 | breast tumor | 2.01 |
| breast normal GW00-413 | 28798 | 29.39 | 545.81 | 545.81 | breast normal | |
| breast tumor GW00-412 | 28797 | 29.84 | 419.06 | 838.12 | breast tumor | 1.54 |
| breast normal GW00- | 27592-95 | 29.87 | 412.4 | 412.40 | breast normal | |
| 235:238 | | | | | | |
| breast tumor GW00- | 27588-91 | 29.51 | 506.62 | 506.62 | breast tumor | 1.23 |
| 231:234 | | | | | | |
| breast normal GW98-621 | 23656 | 29.1 | 643.06 | 1286.12 | breast normal | |
| breast tumor GW98-620 | 23655 | 29.49 | 514.85 | 1029.70 | breast tumor | −1.25 |
| brain normal BB99-542 | 25507 | 27.37 | 1746.53 | 3493.06 | brain normal | |
| brain normal BB99-406 | 25509 | 28.07 | 1165 | 2330.00 | brain normal | |
| brain normal BB99-904 | 25546 | 27.12 | 2019.32 | 4038.64 | brain normal | |
| brain stage 5 ALZ BB99- | 25502 | 28.97 | 695.1 | 1390.20 | brain stage 5 ALZ | −2.36 |
| 874 | | | | | | |
| brain stage 5 ALZ BB99- | 25503 | 27.22 | 1896.94 | 3793.88 | brain stage 5 ALZ | 1.15 |
| 887 | | | | | | |
| brain stage 5 ALZ BB99- | 25504 | 27.47 | 1645.13 | 3290.26 | brain stage 5 ALZ | 1.00 |
| 862 | | | | | | |
| brain stage 5 ALZ BB99- | 25542 | 28 | 1212.32 | 2424.64 | brain stage 5 ALZ | −1.36 |
| 927 | | | | | | |
| CT lung | normal | 28.55 | 882.77 | 1765.54 | CT lung Nml | |
| lung 26 | normal | 29.86 | 415.01 | | lung 26 Nml | |
| lung 27 | normal | 35.3 | 18.03 | 18.03 | lung 27 Nml | |
| lung 24 | COPD | 33.39 | 54.36 | 54.36 | lung 24 COPD | −11.28 |
| lung 28 | COPD | 33.35 | 55.57 | 55.57 | lung 28 COPD | −11.03 |
| lung 23 | COPD | 31.82 | 134.44 | 134.44 | lung 23 COPD | −4.56 |
| lung 25 | normal | 33.35 | 55.41 | 55.41 | lung 25 Nml | |
| asthmatic lung ODO3112 | 29321 | 28.8 | 765.75 | 765.75 | asthmatic lung | 1.25 |
| asthmatic lung ODO3433 | 29323 | 28.19 | 1090.08 | 2180.16 | asthmatic lung | 3.56 |
| asthmatic lung ODO3397 | 29322 | 27.26 | 1854.74 | 3709.48 | asthmatic lung | 6.05 |
| asthmatic lung ODO4928 | 29325 | 27.68 | 1457.04 | 2914.08 | asthmatic lung | 4.75 |
| endo cells | control | 34.77 | 24.44 | 24.44 | endo cells | |
| endo VEGF | | 40 | 0 | 0.00 | endo VEGF | −24.44 |
| endo bFGF | | 40 | 0 | 0.00 | endo bFGF | −24.44 |
| heart Clontech | normal | 29.86 | 415.24 | 830.48 | heart | |
| heart (T-1) ischemic | 29417 | 33.68 | 45.87 | 91.74 | heart (T-1) | −9.05 |
| | | | | | ischemic | |
| heart (T-14) non- | 29422 | 31.26 | 185.72 | 371.44 | heart (T-14) non- | −2.24 |
| obstructive DCM | | | | | obstructive DCM | |
| heart (T-3399) DCM | 29426 | 32.44 | 93.97 | 187.94 | heart (T-3399) | −4.42 |
| | | | | | DCM | |
| adenoid GW99-269 | 26162 | 26.91 | 2268.51 | 4537.02 | adenoid | |
| tonsil GW98-280 | 22582 | 25.76 | 4411.66 | 8823.32 | tonsil | |
| T cells PC00314 | 28453 | 25.29 | 5793.97 | 11587.94 | T cells | |
| PBMNC | | 31.07 | 206.88 | 206.88 | PBMNC | |
| monocyte | | 29.66 | 465.72 | 931.44 | monocyte | |
| B cells PC00665 | 28455 | 24.15 | 11177.51 | 22355.02 | B cells | |
| dendritic cells | 28441 | 26.5 | 2884.61 | 5769.22 | dendritic cells | |
| neutrophils | 28440 | 22.68 | 26075.1 | 26075.10 | neutrophils | |
| eosinophils | 28446 | 24.35 | 9936.83 | 19873.66 | eosinophils | |
| BM unstim | | 28.38 | 972.87 | 972.87 | BM unstim | |
| BM stim | treated | 27.74 | 1410.02 | 1410.02 | BM stim | 1.45 |
| osteo dif | treated | 36:43 | 9.41 | 9.41 | osteo dif | 1.53 |
| osteo undif | | 37.17 | 6.15 | 6.15 | osteo undif | |
| chondrocytes | | 34.83 | 23.73 | 59.33 | chondrocytes | |
| OA Synovium IP12/01 | 29462 | 28.04 | 1187.43 | 1187.43 | OA Synovium | |
| OA Synovium NP10/01 | 29461 | 30.04 | 373.5 | 747.00 | OA Synovium | |
| OA Synovium NP57/00 | 28464 | 28.11 | 1137.32 | 2274.64 | OA Synovium | |
| RA Synovium | 28466 | 28.05 | 1181.53 | 2363.06 | RA Synovium | |
| NP03/01 | | | | | | |
| RA Synovium | 28467 | 28.36 | 985.37 | 1970.74 | RA Synovium | |
| NP71/00 | | | | | | |
| RA Synovium | 28475 | 28.04 | 1182.63 | 2365.26 | RA Synovium | |
| NP45/00 | | | | | | |
| OA bone (biobank) | 29217 | 26.12 | 3584.95 | 3584.95 | OA bone | |
| | | | | | (biobank) | |
| OA bone Sample 1 | J. Emory | 30.9 | 228.59 | 457.18 | OA bone | |
| OA bone Sample 2 | J. Emory | 26.59 | 2736.87 | 5473.74 | OA bone | |
| Cartilage (pool) | Normal | 29.13 | 633.05 | 1266.10 | Nml Cartilage | |
| | | | | | (pool) | |
| Cartilage (pool) | OA | 30.3 | 321.92 | 643.84 | OA Cartilage | −1.97 |
| | | | | | (pool) | |
| PBL unifected | 28441 | 25.03 | 6728.29 | 13456.58 | PBL unifected | |
| PBL HIV IIIB | 28442 | 26.51 | 2858.29 | 5716.58 | PBL HIV IIIB | −2.35 |
| MRC5 uninfected | 29158 | 36.86 | 7.36 | 14.72 | MRC5 | |
| (100%) | | | | | uninfected | |
| | | | | | (100%) | |
| MRC5 HSV strain F | 29178 | 30.13 | 355.37 | 710.74 | MRC5 HSV | 48.28 |
| | | | | | strain F | |
| W12 cells | 29179 | 35.49 | 16.22 | 32.44 | W12 cells | 33.10 |
| Keratinocytes | 29180 | 40 | 0.49 | 0.98 | Keratinocytes | |
| B-actin control | | 27.83 | 1341.01 | | | |
| genomic | | 28.49 | 916.45 | | | |
| 1.00E+05 | | 20.6 | 100000 | | | |
| 1.00E+05 | | 20.5 | 100000 | | | |
| 1.00E+04 | | 23.82 | 10000 | | | |
| 1.00E+04 | | 23.85 | 10000 | | | |
| 1.00E+03 | | 28.1 | 1000 | | | |
| 1.00E+03 | | 27.86 | 1000 | | | |
| 1.00E+02 | | 32.6 | 100 | | | |
| 1.00E+02 | | 33.28 | 100 | | | |
| 1.00E+01 | | 36.1 | 10 | | | |
| 1.00E+01 | | 38.23 | 10 | | | |
| 1.00E+00 | | 40 | 0 | | | |
| 1.00E+00 | | 38.73 | 1 | | | |
| NTC | | 40 | 0 | | | |

*lung 26 Normal has been omitted due to multiple amplification failures from that sample

Gene Name sbg1548844slit

| | Fold Change in Disease |
|---|---|
| | Population Relative to |
| Disease tissues | Normal |
| colon tumor | −1.31 |
| colon tumor | −3.16 |
| colon tumor | −3.17 |
| colon tumor | −1.20 |
| lung tumor | −8.57 |
| lung tumor | −4.03 |
| lung tumor | 1.25 |
| lung tumor | −1.60 |
| breast tumor | 2.01 |
| breast tumor | 1.54 |
| breast tumor | 1.23 |
| breast tumor | −1.25 |
| brain stage 5 ALZ | −2.36 |
| brain stage 5 ALZ | 1.15 |
| brain stage 5 ALZ | 1.00 |
| brain stage 5 ALZ | −1.36 |
| lung 24 COPD | −11.28 |
| lung 28 COPD | −11.03 |
| lung 23 COPD | −4.56 |
| asthmatic lung | 1.25 |
| asthmatic lung | 3.56 |
| asthmatic lung | 6.05 |
| asthmatic lung | 4.75 |
| endo VEGF | −24.44 |
| endo bFGF | −24.44 |
| heart (T-1) ischemic | −9.05 |
| heart (T-14) non-obstructive DCM | −2.24 |
| heart (T-3399) DCM | −4.42 |
| BM stim | 1.45 |
| osteo dif | 1.53 |
| OA Cartilage (pool) | −1.97 |
| PBL HIV IIIB | −2.35 |
| MRC5 HSV strain F | 48.28 |
| W12 cells | 33.10 |

Gene Name sbg1545249GGT

Moderate to high overall expression. The highest normal expression is seen in the whole brain, fetal liver, jejunum, and kidney. The highest disease expression is seen in the uninfected PBLs, one of the normal lung samples, two of the lung tumor samples, one of the breast tumor samples, and one of the Alzheimer's brain samples. Upregulation in 1 of 4 asthmatic lung samples with corroborating high expression in the eosinophils and neutrophils indicates a possible involvement in asthma. Upregulated in the stimulated bone marrow and the differentiated osteoblasts. High expression in the RA and OA synovium samples, the OA bone samples, and the chondrocytes with corroborating high expression in T cells, B cells, dendritic cells, eosinophils, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg1545249GGT | 1 and 2) | 1) | 2) | Copies | (ng) | (ng) | RNA |
| Subcutaneous | 31.62, 31.21 | 4.41 | 5.75 | 5.08 | 3.06 | 16.34 | 83.01 |
| Adipocytes Zenbio | | | | | | | |
| Subcutaneous | 33.61, 34.02 | 1.22 | 0.94 | 1.08 | 0.96 | 52.36 | 56.54 |
| Adipose Zenbio | | | | | | | |
| Adrenal Gland Clontech | 34.19, 34.29 | 0.84 | 0.79 | 0.82 | 0.61 | 81.97 | 66.80 |
| Whole Brain Clontech | 24.05, 23.77 | 583.11 | 695.25 | 639.18 | 7.24 | 6.91 | 4414.23 |
| Fetal Brain Clontech | 32.01, 33.17 | 3.44 | 1.63 | 2.54 | 0.48 | 103.95 | 263.51 |
| Cerebellum Clontech | 30.97, 31.22 | 6.7 | 5.72 | 6.21 | 2.17 | 23.04 | 143.09 |
| Cervix | 29.82, 29.96 | 14.13 | 12.87 | 13.50 | 2.42 | 20.66 | 278.93 |
| Colon | 29.01, 28.36 | 23.74 | 36.07 | 29.91 | 2.71 | 18.45 | 551.75 |
| Endometrium | 30.9, 30.51 | 7.04 | 9.01 | 8.03 | 0.73 | 68.21 | 547.41 |
| Esophagus | 32.04, 31.44 | 3.38 | 4.96 | 4.17 | 1.37 | 36.50 | 152.19 |
| Heart Clontech | 32.72, 32.43 | 2.17 | 2.63 | 2.40 | 1.32 | 37.88 | 90.91 |
| Hypothalamus | 35.78, 35.29 | 0.3 | 0.42 | 0.36 | 0.32 | 155.28 | 55.90 |
| Ileum | 30.08, 29.55 | 11.9 | 16.8 | 14.35 | 2.58 | 19.38 | 278.10 |
| Jejunum | 25.01, 24.73 | 313.84 | 375.1 | 344.47 | 6.60 | 7.58 | 2609.62 |
| Kidney | 27.57, 26.71 | 59.97 | 104.97 | 82.47 | 2.12 | 23.58 | 1945.05 |
| Liver | 29.26, 29.01 | 20.27 | 23.78 | 22.03 | 1.50 | 33.33 | 734.17 |
| Fetal Liver Clontech | 23.26, 23.03 | 965.92 | 1122.38 | 1044.15 | 10.40 | 4.81 | 5019.95 |
| Lung | 28.85, 28.96 | 26.44 | 24.61 | 25.53 | 2.57 | 19.46 | 496.60 |
| Mammary Gland | 27.08, 26.49 | 82.59 | 120.38 | 101.49 | 13.00 | 3.85 | 390.33 |
| Clontech | | | | | | | |
| Myometrium | 28.76, 28.82 | 27.96 | 26.89 | 27.43 | 2.34 | 21.37 | 586.00 |
| Omentum | 28.68, 28.19 | 29.4 | 40.44 | 34.92 | 3.94 | 12.69 | 443.15 |
| Ovary | 28.57, 29 | 31.55 | 23.93 | 27.74 | 4.34 | 11.52 | 319.59 |
| Pancreas | 31.3, 32.5 | 5.43 | 2.51 | 3.97 | 0.81 | 61.80 | 245.36 |
| Head of Pancreas | 32.02, 30.57 | 3.43 | 8.69 | 6.06 | 1.57 | 31.85 | 192.99 |
| Parotid Gland | 28.88, 28.71 | 25.9 | 28.77 | 27.34 | 5.48 | 9.12 | 249.41 |
| Placenta Clontech | 29.76, 29.92 | 14.66 | 13.19 | 13.93 | 5.26 | 9.51 | 132.37 |
| Prostate | 27.67, 27.3 | 56.27 | 71.35 | 63.81 | 3.00 | 16.67 | 1063.50 |
| Rectum | 30.25, 29.97 | 10.66 | 12.85 | 11.76 | 1.23 | 40.65 | 477.85 |
| Salivary Gland Clontech | 28.72, 28.84 | 28.61 | 26.59 | 27.60 | 7.31 | 6.84 | 188.78 |
| Skeletal Muscle | 32.84, 33.59 | 2.01 | 1.24 | 1.63 | 1.26 | 39.68 | 64.48 |
| Clontech | | | | | | | |
| Skin | 32.24, 32.02 | 2.97 | 3.42 | 3.20 | 1.21 | 41.32 | 132.02 |
| Small Intestine Clontech | 32.61, 33.84 | 2.33 | 1.06 | 1.70 | 0.98 | 51.07 | 86.57 |
| Spleen | 29.4, 29.09 | 18.46 | 22.52 | 20.49 | 4.92 | 10.16 | 208.23 |
| Stomach | 29.3, 28.98 | 19.69 | 24.22 | 21.96 | 2.73 | 18.32 | 402.11 |
| Testis Clontech | 29.21, 28.71 | 20.9 | 28.86 | 24.88 | 0.57 | 87.87 | 2186.29 |
| Thymus Clontech | 25.72, 25.66 | 197.6 | 206.04 | 201.82 | 9.89 | 5.06 | 1020.32 |
| Thyroid | 28.92, 28.47 | 25.22 | 33.64 | 29.43 | 2.77 | 18.05 | 531.23 |
| Trachea Clontech | 27.95, 27.51 | 47.11 | 62.56 | 54.84 | 9.71 | 5.15 | 282.36 |
| Urinary Bladder | 31.23, 30.92 | 5.69 | 6.94 | 6.32 | 5.47 | 9.14 | 57.72 |
| Uterus | 27.58, 27.61 | 59.69 | 58.49 | 59.09 | 5.34 | 9.36 | 553.28 |
| genomic | 22.68 | 1408.8 | | | | | |
| b-actin | 25.39 | 244.89 | | | | | |
| 1.00E+05 | 16.6 | 100000 | | | | | |
| 1.00E+05 | 16.65 | 100000 | | | | | |
| 1.00E+04 | 19.45 | 10000 | | | | | |
| 1.00E+04 | 19.5 | 10000 | | | | | |
| 1.00E+03 | 22.52 | 1000 | | | | | |
| 1.00E+03 | 22.65 | 1000 | | | | | |
| 1.00E+02 | 26.79 | 100 | | | | | |
| 1.00E+02 | 26.49 | 100 | | | | | |
| 1.00E+01 | 30.57 | 10 | | | | | |
| 1.00E+01 | 30.13 | 10 | | | | | |
| 1.00E+00 | 34.07 | 1 | | | | | |
| 1.00E+00 | 34.53 | 1 | | | | | |
| NTC | 33.14 | −1 | | | | | |
| NTC | 34.02 | −1 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1545249GGT | identifier) | Ct | copies | RNA | Sample | Population |
| colon normal GW98-167 | 21941 | 24.12 | 568.58 | 1137.16 | colon normal | |
| colon tumor GW98-166 | 21940 | 22.66 | 1447.09 | 2894.18 | colon tumor | 2.55 |
| colon normal GW98-178 | 22080 | 27.35 | 72.47 | 144.94 | colon normal | |
| colon tumor GW98-177 | 22060 | 26.11 | 159.63 | 319.26 | colon tumor | 2.20 |
| colon normal GW98-561 | 23514 | 24.45 | 461 | 922.00 | colon normal | |
| colon tumor GW98-560 | 23513 | 25.46 | 241.41 | 482.82 | colon tumor | −1.91 |
| colon normal GW98-894 | 24691 | 25.3 | 268.35 | 536.70 | colon normal | |
| colon tumor GW98-893 | 24690 | 24.57 | 426.88 | 853.76 | colon tumor | 1.59 |
| lung normal GW98-3 | 20742 | 21.23 | 3603.05 | 7206.10 | lung normal | |
| lung tumor GW98-2 | 20741 | 23.74 | 727.24 | 1454.48 | lung tumor | −4.95 |
| lung normal GW97-179 | 20677 | 22.44 | 1667.66 | 3335.32 | lung normal | |
| lung tumor GW97-178 | 20676 | 21.12 | 3870.78 | 7741.56 | lung tumor | 2.32 |
| lung normal GW98-165 | 21922 | 21.5 | 3037.83 | 6075.66 | lung normal | |
| lung tumor GW98-164 | 21921 | 23.2 | 1023.32 | 2046.64 | lung tumor | −2.97 |
| lung normal GW98-282 | 22584 | 23.03 | 1140.35 | 2280.70 | lung normal | |
| lung tumor GW98-281 | 22583 | 23.37 | 918.94 | 1837.88 | lung tumor | −1.24 |
| breast normal GW00-392 | 28750 | 24.18 | 549.13 | 549.13 | breast normal | |
| breast tumor GW00-391 | 28746 | 23.86 | 673.37 | 1346.74 | breast tumor | 2.45 |
| breast normal GW00-413 | 28798 | 24.69 | 396.34 | 396.34 | breast normal | |
| breast tumor GW00-412 | 28797 | 25 | 325.41 | 650.82 | breast tumor | 1.64 |
| breast normal GW00- | 27592-95 | 24.78 | 372.82 | 372.82 | breast normal | |
| 235:238 | | | | | | |
| breast tumor GW00- | 27588-91 | 24.73 | 386.53 | 386.53 | breast tumor | 1.04 |
| 231:234 | | | | | | |
| breast normal GW98-621 | 23656 | 23.2 | 1026.45 | 2052.90 | breast normal | |
| breast tumor GW98-620 | 23655 | 21.15 | 3793.2 | 7586.40 | breast tumor | 3.70 |
| brain normal BB99-542 | 25507 | 24.88 | 349.22 | 698.44 | brain normal | |
| brain normal BB99-406 | 25509 | 24.34 | 493.57 | 987.14 | brain normal | |
| brain normal BB99-904 | 25546 | 25.81 | 193.58 | 387.16 | brain normal | |
| brain stage 5 ALZ BB99- | 25502 | 25.38 | 254.26 | 508.52 | brain stage 5 ALZ | −1.36 |
| 874 | | | | | | |
| brain stage 5 ALZ BB99- | 25503 | 24.47 | 455.16 | 910.32 | brain stage 5 ALZ | 1.32 |
| 887 | | | | | | |
| brain stage 5 ALZ BB99- | 25504 | 24.84 | 359.61 | 719.22 | brain stage 5 ALZ | 1.04 |
| 862 | | | | | | |
| brain stage 5 ALZ BB99- | 25542 | 24.4 | 474.46 | 948.92 | brain stage 5 ALZ | 1.37 |
| 927 | | | | | | |
| CT lung | normal | 21.68 | 2706.57 | 5413.14 | CT lung Nml | |
| lung 26 | normal | 24.34 | 493.3 | | lung 26 Nml | |
| lung 27 | normal | 25.81 | 193.15 | 193.15 | lung 27 Nml | |
| lung 24 | COPD | 26.14 | 156.98 | 156.98 | lung 24 COPD | −12.38 |
| lung 28 | COPD | 25.92 | 179.93 | 179.93 | lung 28 COPD | −10.80 |
| lung 23 | COPD | 27.59 | 62.3 | 62.30 | lung 23 COPD | −31.20 |
| lung 25 | normal | 25.57 | 225.7 | 225.70 | lung 25 Nml | |
| asthmatic lung ODO3112 | 29321 | 22.08 | 2095.01 | 2095.01 | asthmatic lung | 1.08 |
| asthmatic lung ODO3433 | 29323 | 23.25 | 989.73 | 1979.46 | asthmatic lung | 1.02 |
| asthmatic lung ODO3397 | 29322 | 21.19 | 3692.45 | 7384.90 | asthmatic lung | 3.80 |
| asthmatic lung ODO4928 | 29325 | 23.47 | 859.31 | 1718.62 | asthmatic lung | −1.13 |
| endo cells | control | 30.71 | 8.5 | 8.50 | endo cells | |
| endo VEGF | | 30.51 | 9.6 | 9.60 | endo VEGF | 1.13 |
| endo bFGF | | 31.09 | 6.64 | 6.64 | endo bFGF | −1.28 |
| heart Clontech | normal | 27.98 | 48.49 | 96.98 | heart | |
| heart (T-1) ischemic | 29417 | 27.29 | 75.35 | 150.70 | heart (T-1) | 1.55 |
| | | | | | ischemic | |
| heart (T-14) non- | 29422 | 28.39 | 37.3 | 74.60 | heart (T-14) non- | −1.30 |
| obstructive DCM | | | | | obstructive DCM | |
| heart (T-3399) DCM | 29426 | 27.38 | 71.19 | 142.38 | heart (T-3399) | 1.47 |
| | | | | | DCM | |
| adenoid GW99-269 | 26162 | 26.4 | 133.14 | 266.28 | adenoid | |
| tonsil GW98-280 | 22582 | 24.56 | 428.49 | 856.98 | tonsil | |
| T cells PC00314 | 28453 | 25.39 | 252.86 | 505.72 | T cells | |
| PBMNC | | 29.02 | 24.87 | 24.87 | PBMNC | |
| monocyte | | 28.26 | 40.37 | 80.74 | monocyte | |
| B cells PC00665 | 28455 | 26.08 | 162.64 | 325.28 | B cells | |
| dendritic cells | 28441 | 24.48 | 453.66 | 907.32 | dendritic cells | |
| neutrophils | 28440 | 23.63 | 775.8 | 775.80 | neutrophils | |
| eosinophils | 28446 | 23.83 | 682.91 | 1365.82 | eosinophils | |
| BM unstim | | 27.38 | 71.14 | 71.14 | BM unstim | |
| BM stim | treated | 24.51 | 443.54 | 443.54 | BM stim | 6.23 |
| osteo dif | treated | 29.33 | 20.41 | 20.41 | osteo dif | 21.95 |
| osteo undif | | 34.16 | 0.93 | 0.93 | osteo undif | |
| chondrocytes | | 27.1 | 85.11 | 212.78 | chondrocytes | |
| OA Synovium IP12/01 | 29462 | 24.06 | 590.18 | 590.18 | OA Synovium | |
| OA Synovium NP10/01 | 29461 | 26.38 | 134.76 | 269.52 | OA Synovium | |
| OA Synovium NP57/00 | 28464 | 24.58 | 425.07 | 850.14 | OA Synovium | |
| RA Synovium | 28466 | 25.32 | 264.96 | 529.92 | RA Synovium | |
| NP03/01 | | | | | | |
| RA Synovium | 28467 | 24.32 | 500.45 | 1000.90 | RA Synovium | |
| NP71/00 | | | | | | |
| RA Synovium | 28475 | 24.47 | 454.68 | 909.36 | RA Synovium | |
| NP45/00 | | | | | | |
| OA bone (biobank) | 29217 | 24.82 | 363.24 | 363.24 | OA bone (biobank) | |
| OA bone Sample 1 | J. Emory | 24.42 | 468.77 | 937.54 | OA bone | |
| OA bone Sample 2 | J. Emory | 25.35 | 259.37 | 518.74 | OA bone | |
| Cartilage (pool) | Normal | 26.17 | 153.58 | 307.16 | Nml Cartilage | |
| | | | | | (pool) | |
| Cartilage (pool) | OA | 27.29 | 75.26 | 150.52 | OA Cartilage (pool) | −2.04 |
| PBL unifected | 28441 | 21.15 | 3789.69 | 7579.38 | PBL unifected | |
| PBL HIV IIIB | 28442 | 23.03 | 1144.57 | 2289.14 | PBL HIV IIIB | −3.31 |
| MRC5 uninfected | 29158 | 25.59 | 222.21 | 444.42 | MRC5 uninfected | |
| (100%) | | | | | (100%) | |
| MRC5 HSV strain F | 29178 | 27.31 | 74.06 | 148.12 | MRC5 HSV strain | −3.00 |
| | | | | | F | |
| W12 cells | 29179 | 26.66 | 112.62 | 225.24 | W12 cells | −2.22 |
| Keratinocytes | 29180 | 25.41 | 249.47 | 498.94 | Keratinocytes | |
| B-actin control | | 24.63 | 410.6 | | | |
| genomic | | 22.01 | 2185.62 | | | |
| 1.00E+05 | | 16.42 | 100000 | | | |
| 1.00E+05 | | 16.27 | 100000 | | | |
| 1.00E+04 | | 19.39 | 10000 | | | |
| 1.00E+04 | | 19.35 | 10000 | | | |
| 1.00E+03 | | 22.78 | 1000 | | | |
| 1.00E+03 | | 22.61 | 1000 | | | |
| 1.00E+02 | | 26.76 | 100 | | | |
| 1.00E+02 | | 27.24 | 100 | | | |
| 1.00E+01 | | 31.24 | 10 | | | |
| 1.00E+01 | | 31.15 | 10 | | | |
| 1.00E+00 | | 34.14 | 1 | | | |
| 1.00E+00 | | 33.14 | 1 | | | |
| NTC | | 36.02 | −1 | | | |

*lung 26 Normal has been omitted due to multiple amplification failures from that sample

Gene Name sbg1545249GGT

| | Fold Change in Disease |
|---|---|
| | Population Relative to |
| Disease tissues | Normal |
| colon tumor | 2.55 |
| colon tumor | 2.20 |
| colon tumor | −1.91 |
| colon tumor | 1.59 |
| lung tumor | −4.95 |
| lung tumor | 2.32 |
| lung tumor | −2.97 |
| lung tumor | −1.24 |
| breast tumor | 2.45 |
| breast tumor | 1.64 |
| breast tumor | 1.04 |
| breast tumor | 3.70 |
| brain stage 5 ALZ | −1.36 |
| brain stage 5 ALZ | 1.32 |
| brain stage 5 ALZ | 1.04 |
| brain stage 5 ALZ | 1.37 |
| lung 24 COPD | −12.38 |
| lung 28 COPD | −10.80 |
| lung 23 COPD | −31.20 |
| asthmatic lung | 1.08 |
| asthmatic lung | 1.02 |
| asthmatic lung | 3.80 |
| asthmatic lung | −1.13 |
| endo VEGF | 1.13 |
| endo bFGF | −1.28 |
| heart (T-1) ischemic | 1.55 |
| heart (T-14) non-obstructive DCM | −1.30 |
| heart (T-3399) DCM | 1.47 |
| BM stim | 6.23 |
| osteo dif | 21.95 |
| OA Cartilage (pool) | −2.04 |
| PBL HIV IIIB | −3.31 |
| MRC5 HSV strain F | −3.00 |
| W12 cells | −2.22 |

Gene Name SBh1813899.Y82017 (Taqman was Performed for this Sample)

Moderate to low overall expression. The highest normal expression is seen in the omentum, prostate, testis, ovary, and uterus. The highest disease expression is seen in the tonsil sample suggesting that this gene may play a role in chronic ear infections and tonsillitis. Upregulation in 2 of 4 lung tumor samples implicates this gene in cancer of lung. Downregulation in 2 of 4 Alzheimer's brain samples suggests an involvement in Alzheimer's disease. Downregulation in the VEGF- and bFGF-treated endothelial cells suggests that this gene may be involved in angiogenesis.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample | (sampl | (sample | GOI | rRNA | 18 S | total |
| SBh1813899.Y82017 | 1 and 2) | 1) | 2) | Copies | (ng) | rRNA(ng) | RNA |
| Subcutaneous | 37.05, 40 | 4.96 | 0 | 2.48 | 3.06 | 16.34 | 40.52 |
| Adipocytes Zenbio | | | | | | | |
| Subcutaneous | 40, 37.67 | 0 | 3.35 | 1.68 | 0.96 | 52.36 | 87.70 |
| Adipose Zenbio | | | | | | | |
| Adrenal Gland Clontech | 34.59, 40 | 23.56 | 0 | 11.78 | 0.61 | 81.97 | 965.57 |
| Whole Brain Clontech | 34.5, 33.89 | 24.97 | 36.63 | 30.80 | 7.24 | 6.91 | 212.71 |
| Fetal Brain Clontech | 40, 40 | 0 | 0 | 0.00 | 0.48 | 103.95 | 0.00 |
| Cerebellum Clontech | 36.14, 37.22 | 8.84 | 4.47 | 6.66 | 2.17 | 23.04 | 153.34 |
| Cervix | 33.95, 33.12 | 35.31 | 59.9 | 47.61 | 2.42 | 20.66 | 983.57 |
| Colon | 34.6, 36.17 | 23.39 | 8.67 | 16.03 | 2.71 | 18.45 | 295.76 |
| Endometrium | 35, 35.05 | 18.22 | 17.67 | 17.95 | 0.73 | 68.21 | 1224.08 |
| Esophagus | 36.54, 35.75 | 6.84 | 11.34 | 9.09 | 1.37 | 36.50 | 331.75 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 35.02, 34.12 | 18 | 31.72 | 24.86 | 2.58 | 19.38 | 481.78 |
| Jejunum | 31.06, 30.59 | 219.67 | 296.53 | 258.10 | 6.60 | 7.58 | 1955.30 |
| Kidney | 40, 40 | 0 | 0 | 0.00 | 2.12 | 23.58 | 0.00 |
| Liver | 35.07, 33.33 | 17.44 | 52.2 | 34.82 | 1.50 | 33.33 | 1160.67 |
| Fetal Liver Clontech | 40, 37.65 | 0 | 3.4 | 1.70 | 10.40 | 4.81 | 8.17 |
| Lung | 36.1, 34.93 | 9.09 | 18.99 | 14.04 | 2.57 | 19.46 | 273.15 |
| Mammary Gland | 37.41, 37.9 | 3.95 | 2.9 | 3.43 | 13.00 | 3.85 | 13.17 |
| Clontech | | | | | | | |
| Myometrium | 36.92, 37.18 | 5.4 | 4.57 | 4.99 | 2.34 | 21.37 | 106.52 |
| Omentum | 28.23, 27.82 | 1321.7 | 1711.31 | 1516.51 | 3.94 | 12.69 | 19244.99 |
| Ovary | 30.87, 31.14 | 247.95 | 208.87 | 228.41 | 4.34 | 11.52 | 2631.45 |
| Pancreas | 37.13, 40 | 4.72 | 0 | 2.36 | 0.81 | 61.80 | 145.86 |
| Head of Pancreas | 40, 37.55 | 0 | 3.61 | 1.81 | 1.57 | 31.85 | 57.48 |
| Parotid Gland | 31.13, 32.45 | 210.06 | 91.6 | 150.83 | 5.48 | 9.12 | 1376.19 |
| Placenta Clontech | 34.44, 35.13 | 25.95 | 16.77 | 21.36 | 5.26 | 9.51 | 203.04 |
| Prostate | 28.45, 28.4 | 1151.04 | 1187.63 | 1169.34 | 3.00 | 16.67 | 19488.92 |
| Rectum | 38.08, 37.37 | 2.58 | 4.06 | 3.32 | 1.23 | 40.65 | 134.96 |
| Salivary Gland | 33.44, 32.39 | 48.94 | 94.81 | 71.88 | 7.31 | 6.84 | 491.62 |
| Clontech | | | | | | | |
| Skeletal Muscle | 40, 40 | 0 | 0 | 0.00 | 1.26 | 39.68 | 0.00 |
| Clontech | | | | | | | |
| Skin | 35.78, 37.12 | 11.08 | 4.74 | 7.91 | 1.21 | 41.32 | 326.86 |
| Small Intestine | 40, 40 | 0 | 0 | 0.00 | 0.98 | 51.07 | 0.00 |
| Clontech | | | | | | | |
| Spleen | 33.64, 33.21 | 43.03 | 56.35 | 49.69 | 4.92 | 10.16 | 504.98 |
| Stomach | 32.31, 31.4 | 99.53 | 178.03 | 138.78 | 2.73 | 18.32 | 2541.76 |
| Testis Clontech | 32.34, 32.33 | 98.13 | 98.48 | 98.31 | 0.57 | 87.87 | 8638.40 |
| Thymus Clontech | 37.44, 35.09 | 3.88 | 17.16 | 10.52 | 9.89 | 5.06 | 53.19 |
| Thyroid | 35.52, 38.84 | 13.12 | 1.6 | 7.36 | 2.77 | 18.05 | 132.85 |
| Trachea Clontech | 30.8, 29.83 | 259.07 | 480.34 | 369.71 | 9.71 | 5.15 | 1903.73 |
| Urinary Bladder | 40, 35.78 | 0 | 11.08 | 5.54 | 5.47 | 9.14 | 50.64 |
| Uterus | 30.59, 29.63 | 297.31 | 545 | 421.16 | 5.34 | 9.36 | 3943.40 |
| genomic | 27.5 | 2091.83 | | | | | |
| b-actin | 29.13 | 745.39 | | | | | |
| 1.00E+05 | 21.9 | 100000 | | | | | |
| 1.00E+05 | 21.69 | 100000 | | | | | |
| 1.00E+04 | 24.81 | 10000 | | | | | |
| 1.00E+04 | 25.02 | 10000 | | | | | |
| 1.00E+03 | 28.21 | 1000 | | | | | |
| 1.00E+03 | 28.11 | 1000 | | | | | |
| 1.00E+02 | 31.91 | 100 | | | | | |
| 1.00E+02 | 32.21 | 100 | | | | | |
| 1.00E+01 | 35.06 | 10 | | | | | |
| 1.00E+01 | 37.77 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| SBh1813899.Y82017 | identifier) | Ct | copies | RNA | Sample | Population |
| colon normal GW98-167 | 21941 | 38.12 | 1.03 | 2.06 | colon normal | |
| colon tumor GW98-166 | 21940 | 37.49 | 1.58 | 3.16 | colon tumor | 1.53 |
| colon normal GW98-178 | 22080 | 35.97 | 4.43 | 8.86 | colon normal | |
| colon tumor GW98-177 | 22060 | 38.1 | 1.04 | 2.08 | colon tumor | −4.26 |
| colon normal GW98-561 | 23514 | 38.75 | 0.67 | 1.34 | colon normal | |
| colon tumor GW98-560 | 23513 | 39.01 | 0.56 | 1.12 | colon tumor | −1.20 |
| colon normal GW98-894 | 24691 | 33.57 | 22.61 | 45.22 | colon normal | |
| colon tumor GW98-893 | 24690 | 36.96 | 2.25 | 4.50 | colon tumor | −10.05 |
| lung normal GW98-3 | 20742 | 38.56 | 0.76 | 1.52 | lung normal | |
| lung tumor GW98-2 | 20741 | 31.65 | 82.7 | 165.40 | lung tumor | 108.82 |
| lung normal GW97-179 | 20677 | 34.04 | 16.4 | 32.80 | lung normal | |
| lung tumor GW97-178 | 20676 | 37.01 | 2.19 | 4.38 | lung tumor | −7.49 |
| lung normal GW98-165 | 21922 | 40 | 0 | 0.00 | lung normal | |
| lung tumor GW98-164 | 21921 | 34.02 | 16.62 | 33.24 | lung tumor | 33.24 |
| lung normal GW98-282 | 22584 | 31.32 | 103.46 | 206.92 | lung normal | |
| lung tumor GW98-281 | 22583 | 34.18 | 14.87 | 29.74 | lung tumor | −6.96 |
| breast normal GW00-392 | 28750 | 32.79 | 38.17 | 38.17 | breast normal | |
| breast tumor GW00-391 | 28746 | 35.08 | 8.09 | 16.18 | breast tumor | −2.36 |
| breast normal GW00-413 | 28798 | 35.02 | 8.4 | 8.40 | breast normal | |
| breast tumor GW00-412 | 28797 | 34.18 | 14.89 | 29.78 | breast tumor | 3.55 |
| breast normal GW00- | 27592-95 | 32.3 | 53.22 | 53.22 | breast normal | |
| 235:238 | | | | | | |
| breast tumor GW00- | 27588-91 | 36.61 | 2.87 | 2.87 | breast tumor | −18.54 |
| 231:234 | | | | | | |
| breast normal GW98-621 | 23656 | 34.23 | 14.44 | 28.88 | breast normal | |
| breast tumor GW98-620 | 23655 | 32.23 | 56.07 | 112.14 | breast tumor | 3.88 |
| brain normal BB99-542 | 25507 | 33.42 | 24.93 | 49.86 | brain normal | |
| brain normal BB99-406 | 25509 | 36.23 | 3.71 | 7.42 | brain normal | |
| brain normal BB99-904 | 25546 | 40 | 0 | 0.00 | brain normal | |
| brain stage 5 ALZ BB99- | 25502 | 40 | 0 | 0.00 | brain stage 5 ALZ | −19.09 |
| 874 | | | | | | |
| brain stage 5 ALZ BB99- | 25503 | 40 | 0 | 0.00 | brain stage 5 ALZ | −19.09 |
| 887 | | | | | | |
| brain stage 5 ALZ BB99- | 25504 | 36.12 | 4 | 8.00 | brain stage 5 ALZ | −2.39 |
| 862 | | | | | | |
| brain stage 5 ALZ BB99- | 25542 | 35.23 | 7.29 | 14.58 | brain stage 5 ALZ | −1.31 |
| 927 | | | | | | |
| CT lung | normal | 37.65 | 1.42 | 2.84 | CT lung Nml | |
| lung 26 | normal | 36.16 | 3.88 | | lung 26 Nml | |
| lung 27 | normal | 40 | 0 | 0.00 | lung 27 Nml | |
| lung 24 | COPD | 38.06 | 1.07 | 1.07 | lung 24 COPD | −1.63 |
| lung 28 | COPD | 35.07 | 8.13 | 8.13 | lung 28 COPD | 4.65 |
| lung 23 | COPD | 35.22 | 7.34 | 7.34 | lung 23 COPD | 4.20 |
| lung 25 | normal | 36.87 | 2.4 | 2.40 | lung 25 Nml | |
| asthmatic lung ODO3112 | 29321 | 37.56 | 1.51 | 1.51 | asthmatic lung | −1.16 |
| asthmatic lung ODO3433 | 29323 | 40 | 0 | 0.00 | asthmatic lung | −1.75 |
| asthmatic lung ODO3397 | 29322 | 38.09 | 1.05 | 2.10 | asthmatic lung | 1.20 |
| asthmatic lung ODO4928 | 29325 | 40 | 0 | 0.00 | asthmatic lung | −1.75 |
| endo cells | control | 35.81 | 4.94 | 4.94 | endo cells | |
| endo VEGF | | 40 | 0 | 0.00 | endo VEGF | −4.94 |
| endo bFGF | | 40 | 0 | 0.00 | endo bFGF | −4.94 |
| heart Clontech | normal | 40 | 0 | 0.00 | heart | |
| heart (T-1) ischemic | 29417 | 40 | 0 | 0.00 | heart (T-1) | 0.00 |
| | | | | | ischemic | |
| heart (T-14) non- | 29422 | 40 | 0 | 0.00 | heart (T-14) non- | 0.00 |
| obstructive DCM | | | | | obstructive DCM | |
| heart (T-3399) DCM | 29426 | 40 | 0 | 0.00 | heart (T-3399) | 0.00 |
| | | | | | DCM | |
| adenoid GW99-269 | 26162 | 32.04 | 63.54 | 127.08 | adenoid | |
| tonsil GW98-280 | 22582 | 29.94 | 264.84 | 529.68 | tonsil | |
| T cells PC00314 | 28453 | 40 | 0 | 0.00 | T cells | |
| PBMNC | | 37.57 | 1.49 | 1.49 | PBMNC | |
| monocyte | | 36.8 | 2.52 | 5.04 | monocyte | |
| B cells PC00665 | 28455 | 32.1 | 61.2 | 122.40 | B cells | |
| dendritic cells | 28441 | 40 | 0 | 0.00 | dendritic cells | |
| neutrophils | 28440 | 32.84 | 36.87 | 36.87 | neutrophils | |
| eosinophils | 28446 | 40 | 0 | 0.00 | eosinophils | |
| BM unstim | | 37.22 | 1.89 | 1.89 | BM unstim | |
| BM stim | treated | 36.25 | 3.65 | 3.65 | BM stim | 1.93 |
| osteo dif | treated | 40 | 0 | 0.00 | osteo dif | −1.67 |
| osteo undif | | 37.41 | 1.67 | 1.67 | osteo undif | |
| chondrocytes | | 34.73 | 10.28 | 25.70 | chondrocytes | |
| OA Synovium IP12/01 | 29462 | 35.3 | 6.98 | 6.98 | OA Synovium | |
| OA Synovium NP10/01 | 29461 | 39.02 | 0.56 | 1.12 | OA Synovium | |
| OA Synovium NP57/00 | 28464 | 33.26 | 27.78 | 55.56 | OA Synovium | |
| RA Synovium | 28466 | 34.81 | 9.69 | 19.38 | RA Synovium | |
| NP03/01 | | | | | | |
| RA Synovium | 28467 | 35.74 | 5.18 | 10.36 | RA Synovium | |
| NP71/00 | | | | | | |
| RA Synovium | 28475 | 33.48 | 23.88 | 47.76 | RA Synovium | |
| NP45/00 | | | | | | |
| OA bone (biobank) | 29217 | 35.34 | 6.79 | 6.79 | OA bone | |
| | | | | | (biobank) | |
| OA bone Sample 1 | J. Emory | 34.06 | 16.14 | 32.28 | OA bone | |
| OA bone Sample 2 | J. Emory | 33.91 | 17.89 | 35.78 | OA bone | |
| Cartilage (pool) | Normal | 40 | 0 | 0.00 | Nml Cartilage | |
| | | | | | (pool) | |
| Cartilage (pool) | OA | 40 | 0 | 0.00 | OA Cartilage | 0.00 |
| | | | | | (pool) | |
| PBL unifected | 28441 | 36.27 | 3.6 | 7.20 | PBL unifected | |
| PBL HIV IIIB | 28442 | 35.52 | 5.99 | 11.98 | PBL HIV IIIB | 1.66 |
| MRC5 uninfected | 29158 | 40 | 0 | 0.00 | MRC5 | |
| (100%) | | | | | uninfected | |
| | | | | | (100%) | |
| MRC5 HSV strain F | 29178 | 37.72 | 1.35 | 2.70 | MRC5 HSV | 2.70 |
| | | | | | strain F | |
| W12 cells | 29179 | 40 | 0 | 0.00 | W12 cells | 0.00 |
| Keratinocytes | 29180 | 40 | 0 | 0.00 | Keratinocytes | |
| B-actin control | | 29.71 | 309.67 | | | |
| genomic | | 28.32 | 792.66 | | | |
| 1.00E+05 | | 21.34 | 100000 | | | |
| 1.00E+05 | | 21.16 | 100000 | | | |
| 1.00E+04 | | 24.15 | 10000 | | | |
| 1.00E+04 | | 24.13 | 10000 | | | |
| 1.00E+03 | | 27.36 | 1000 | | | |
| 1.00E+03 | | 27.69 | 1000 | | | |
| 1.00E+02 | | 33.07 | 100 | | | |
| 1.00E+02 | | 32.04 | 100 | | | |
| 1.00E+01 | | 35.15 | 10 | | | |
| 1.00E+01 | | 35.4 | 10 | | | |
| 1.00E+00 | | 37.34 | 1 | | | |
| 1.00E+00 | | 37.29 | 1 | | | |
| NTC | | 40 | 0 | | | |

*lung 26 Normal has been omitted due to multiple amplification failures from that sample

Gene Name SBh1813899.Y82017

| | Fold Change in Disease |
|---|---|
| | Population Relative to |
| Disease tissues | Normal |
| colon tumor | 1.53 |
| colon tumor | −4.26 |
| colon tumor | −1.20 |
| colon tumor | −10.05 |
| lung tumor | 108.82 |
| lung tumor | −7.49 |
| lung tumor | 33.24 |
| lung tumor | −6.96 |
| breast tumor | −2.36 |
| breast tumor | 3.55 |
| breast tumor | −18.54 |
| breast tumor | 3.88 |
| brain stage 5 ALZ | −19.09 |
| brain stage 5 ALZ | −19.09 |
| brain stage 5 ALZ | −2.39 |
| brain stage 5 ALZ | −1.31 |
| lung 24 COPD | −1.63 |
| lung 28 COPD | 4.65 |
| lung 23 COPD | 4.20 |
| asthmatic lung | −1.16 |
| asthmatic lung | −1.75 |
| asthmatic lung | 1.20 |
| asthmatic lung | −1.75 |
| endo VEGF | −4.94 |
| endo bFGF | −4.94 |
| heart (T-1) ischemic | 0.00 |
| heart (T-14) non-obstructive DCM | 0.00 |
| heart (T-3399) DCM | 0.00 |
| BM stim | 1.93 |
| osteo dif | −1.67 |
| OA Cartilage (pool) | 0.00 |
| PBL HIV IIIB | 1.66 |
| MRC5 HSV strain F | 2.70 |
| W12 cells | 0.00 |

Gene Name sbg1536324mapkkk

Moderate overall expression. The highest normal expression is seen in the whole brain, cerebellum, and kidney as well as many of the tissues representing the GI tract including the colon, jejunum, rectum, and stomach. The highest disease expression is seen in many of the tumor samples and their matched normal counterparts as well as in the uninfected peripheral blood lymphocytes, W12 cells, and the normal keratinocytes. Significant upregulation in 1 of 4 colon tumor samples is sufficient to implicate this gene in colon cancer. Upregulation in 1 of 4 asthmatic lung samples with corroborating high expression in the eosinophils and neutrophils indicates a possible involvement in asthma. Upregulation in 2 of 3 disease heart samples implies an involvement in cardiovascular diseases such as non-obstructive and obstructive DCM. Upregulated in the stimulated bone marrow and the differentiated osteoblasts. Downregulation in the HIV-infected PBLs suggests that this gene may be a host factor in HIV. High expression in the RA and OA synovium samples, the OA bone samples, and the chondrocytes with corroborating high expression in T cells, B cells, eosinophils, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | | detected/ |
| Sample | Ct | copies | copies | Average | 18 S | 50 ng/ | 50 ng |
| sbg1536324- | (sample | (sample | (sample | GOI | rRNA | 18 S | total |
| mapkkk | 1 and 2) | 1) | 2) | Copies | (ng) | rRNA(ng) | RNA |
| Subcutaneous | 35.13, 34.95 | 8.06 | 9.01 | 8.54 | 3.06 | 16.34 | 139.46 |
| Adipocytes Zenbio | | | | | | | |
| Subcutaneous Adipose | 40, 36.08 | 0 | 4.53 | 2.27 | 0.96 | 52.36 | 118.59 |
| Zenbio | | | | | | | |
| Adrenal Gland Clontech | 35.54, 35.09 | 6.31 | 8.3 | 7.31 | 0.61 | 81.97 | 598.77 |
| Whole Brain Clontech | 24.8, 25.05 | 4253.95 | 3672.88 | 3963.42 | 7.24 | 6.91 | 27371.65 |
| Fetal Brain Clontech | 36.36, 40 | 3.83 | 0.25 | 2.04 | 0.48 | 103.95 | 212.06 |
| Cerebellum Clontech | 30.47, 30.18 | 136.52 | 162.55 | 149.54 | 2.17 | 23.04 | 3445.51 |
| Cervix | 33.31, 33.02 | 24.3 | 28.99 | 26.65 | 2.42 | 20.66 | 550.52 |
| Colon | 29.65, 30.3 | 224.17 | 151.39 | 187.78 | 2.71 | 18.45 | 3464.58 |
| Endometrium | 34.05, 33.15 | 15.6 | 26.93 | 21.27 | 0.73 | 68.21 | 1450.55 |
| Esophagus | 34.5, 36.18 | 11.85 | 4.28 | 8.07 | 1.37 | 36.50 | 294.34 |
| Heart Clontech | 40, 40 | 0 | 0 | 0.00 | 1.32 | 37.88 | 0.00 |
| Hypothalamus | 34.47, 40 | 12.03 | 0 | 6.02 | 0.32 | 155.28 | 934.01 |
| Ileum | 32.38, 31.42 | 42.91 | 76.52 | 59.72 | 2.58 | 19.38 | 1157.27 |
| Jejunum | 27.49, 27.23 | 833.68 | 976.73 | 905.21 | 6.60 | 7.58 | 6857.61 |
| Kidney | 31.02, 29.54 | 98.02 | 239.91 | 168.97 | 2.12 | 23.58 | 3985.02 |
| Liver | 33.09, 32.88 | 27.88 | 31.55 | 29.72 | 1.50 | 33.33 | 990.50 |
| Fetal Liver Clontech | 30.28, 30.65 | 153.69 | 122.23 | 137.96 | 10.40 | 4.81 | 663.27 |
| Lung | 31.61, 31.72 | 68.47 | 64.09 | 66.28 | 2.57 | 19.46 | 1289.49 |
| Mammary Gland | 29.5, 28.61 | 245.39 | 421.31 | 333.35 | 13.00 | 3.85 | 1282.12 |
| Clontech | | | | | | | |
| Myometrium | 32.34, 31.29 | 44.01 | 83 | 63.51 | 2.34 | 21.37 | 1356.94 |
| Omentum | 30.04, 30.7 | 176.89 | 118.57 | 147.73 | 3.94 | 12.69 | 1874.75 |
| Ovary | 34.11, 32.17 | 15.03 | 48.55 | 31.79 | 4.34 | 11.52 | 366.24 |
| Pancreas | 32.13, 33.42 | 49.79 | 22.73 | 36.26 | 0.81 | 61.80 | 2241.04 |
| Head of Pancreas | 32.95, 31.15 | 30.39 | 90.67 | 60.53 | 1.57 | 31.85 | 1927.71 |
| Parotid Gland | 29.81, 31.42 | 204.18 | 76.85 | 140.52 | 5.48 | 9.12 | 1282.07 |
| Placenta Clontech | 35.22, 34.15 | 7.65 | 14.65 | 11.15 | 5.26 | 9.51 | 105.99 |
| Prostate | 32.64, 32.53 | 36.51 | 39.11 | 37.81 | 3.00 | 16.67 | 630.17 |
| Rectum | 30.98, 31.06 | 100.38 | 95.28 | 97.83 | 1.23 | 40.65 | 3976.83 |
| Salivary Gland | 30.76, 31.26 | 114.72 | 84.39 | 99.56 | 7.31 | 6.84 | 680.95 |
| Clontech | | | | | | | |
| Skeletal Muscle | 40, 35.91 | 0 | 5.03 | 2.52 | 1.26 | 39.68 | 99.80 |
| Clontech | | | | | | | |
| Skin | 38.91, 35.82 | 0.82 | 5.32 | 3.07 | 1.21 | 41.32 | 126.86 |
| Small Intestine | 33.09, 33.17 | 27.87 | 26.49 | 27.18 | 0.98 | 51.07 | 1388.15 |
| Clontech | | | | | | | |
| Spleen | 31.76, 33.26 | 62.51 | 25.06 | 43.79 | 4.92 | 10.16 | 444.97 |
| Stomach | 30.21, 29.84 | 160.09 | 200.01 | 180.05 | 2.73 | 18.32 | 3297.62 |
| Testis Clontech | 35.2, 34.14 | 7.76 | 14.71 | 11.24 | 0.57 | 87.87 | 987.26 |
| Thymus Clontech | 29.58, 29.01 | 234.48 | 330.52 | 282.50 | 9.89 | 5.06 | 1428.21 |
| Thyroid | 32.1, 32.33 | 50.8 | 44.07 | 47.44 | 2.77 | 18.05 | 856.23 |
| Trachea Clontech | 30.02, 29.71 | 179.68 | 216.47 | 198.08 | 9.71 | 5.15 | 1019.95 |
| Urinary Bladder | 34.48, 32.46 | 12.01 | 40.72 | 26.37 | 5.47 | 9.14 | 241.00 |
| Uterus | 30.3, 30.08 | 151.8 | 173.03 | 162.42 | 5.34 | 9.36 | 1520.74 |
| genomic | 27.06 | 1078.79 | | | | | |
| b-actin | 28.03 | 601.21 | | | | | |
| 1.00E+05 | 19.63 | 100000 | | | | | |
| 1.00E+05 | 19.9 | 100000 | | | | | |
| 1.00E+04 | 23.38 | 10000 | | | | | |
| 1.00E+04 | 23.39 | 10000 | | | | | |
| 1.00E+03 | 26.84 | 1000 | | | | | |
| 1.00E+03 | 27.04 | 1000 | | | | | |
| 1.00E+02 | 31.03 | 100 | | | | | |
| 1.00E+02 | 30.67 | 100 | | | | | |
| 1.00E+01 | 33.53 | 10 | | | | | |
| 1.00E+01 | 36.48 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1536324- | (GSK | | GOI | total | | Disease |
| mapkkk | identifier) | Ct | copies | RNA | Sample | Population |
| colon normal GW98-167 | 21941 | 25.38 | 3449.66 | 6899.32 | colon normal | |
| colon tumor GW98-166 | 21940 | 25.74 | 2747.28 | 5494.56 | colon tumor | −1.26 |
| colon normal GW98-178 | 22080 | 31.04 | 102.2 | 204.40 | colon normal | |
| colon tumor GW98-177 | 22060 | 25.14 | 4004.98 | 8009.96 | colon tumor | 39.19 |
| colon normal GW98-561 | 23514 | 25.68 | 2860.49 | 5720.98 | colon normal | |
| colon tumor GW98-560 | 23513 | 25.22 | 3793.08 | 7586.16 | colon tumor | 1.33 |
| colon normal GW98-894 | 24691 | 24.74 | 5130.6 | 10261.20 | colon normal | |
| colon tumor GW98-893 | 24690 | 24.03 | 7978.97 | 15957.94 | colon tumor | 1.56 |
| lung normal GW98-3 | 20742 | 25.87 | 2533.92 | 5067.84 | lung normal | |
| lung tumor GW98-2 | 20741 | 26.05 | 2268.34 | 4536.68 | lung tumor | −1.12 |
| lung normal GW97-179 | 20677 | 24.92 | 4568.82 | 9137.64 | lung normal | |
| lung tumor GW97-178 | 20676 | 25.15 | 3982.17 | 7964.34 | lung tumor | −1.15 |
| lung normal GW98-165 | 21922 | 25.33 | 3555.29 | 7110.58 | lung normal | |
| lung tumor GW98-164 | 21921 | 25.57 | 3053.68 | 6107.36 | lung tumor | −1.16 |
| lung normal GW98-282 | 22584 | 25.8 | 2649.48 | 5298.96 | lung normal | |
| lung tumor GW98-281 | 22583 | 27.59 | 872.2 | 1744.40 | lung tumor | −3.04 |
| breast normal GW00-392 | 28750 | 28.07 | 648.67 | 648.67 | breast normal | |
| breast tumor GW00-391 | 28746 | 27.22 | 1096.31 | 2192.62 | breast tumor | 3.38 |
| breast normal GW00-413 | 28798 | 28.69 | 440.98 | 440.98 | breast normal | |
| breast tumor GW00-412 | 28797 | 28.38 | 533.68 | 1067.36 | breast tumor | 2.42 |
| breast normal GW00- | 27592-95 | 27.2 | 1109.72 | 1109.72 | breast normal | |
| 235:238 | | | | | | |
| breast tumor GW00- | 27588-91 | 28.37 | 536.17 | 536.17 | breast tumor | −2.07 |
| 231:234 | | | | | | |
| breast normal GW98-621 | 23656 | 26.19 | 2085.44 | 4170.88 | breast normal | |
| breast tumor GW98-620 | 23655 | 27.31 | 1037.07 | 2074.14 | breast tumor | −2.01 |
| brain normal BB99-542 | 25507 | 25.25 | 3734.67 | 7469.34 | brain normal | |
| brain normal BB99-406 | 25509 | 26.67 | 1542.92 | 3085.84 | brain normal | |
| brain normal BB99-904 | 25546 | 26.4 | 1829.69 | 3659.38 | brain normal | |
| brain stage 5 ALZ BB99- | 25502 | 28.33 | 549.79 | 1099.58 | brain stag 5 ALZ | −4.31 |
| 874 | | | | | | |
| brain stage 5 ALZ BB99- | 25503 | 26.12 | 2172.52 | 4345.04 | brain stage 5 ALZ | −1.09 |
| 887 | | | | | | |
| brain stage 5 ALZ BB99- | 25504 | 26.39 | 1840.01 | 3680.02 | brain stage 5 ALZ | −1.29 |
| 862 | | | | | | |
| brain stage 5 ALZ BB99- | 25542 | 25.85 | 2571.02 | 5142.04 | brain stage 5 ALZ | 1.09 |
| 927 | | | | | | |
| CT lung | normal | 27.06 | 1215.1 | 2430.20 | CT lung Nml | |
| lung 26 | normal | 28.58 | 472.09 | | lung 26 Nml | |
| lung 27 | normal | 29.77 | 225.55 | 225.55 | lung 27 Nml | |
| lung 24 | COPD | 30.61 | 133.34 | 133.34 | lung 24 COPD | −7.27 |
| lung 28 | COPD | 30.05 | 189.33 | 189.33 | lung 28 COPD | −5.12 |
| lung 23 | COPD | 31.15 | 95.52 | 95.52 | lung 23 COPD | −10.14 |
| lung 25 | normal | 29.6 | 250.5 | 250.50 | lung 25 Nml | |
| asthmatic lung ODO3112 | 29321 | 26.96 | 1285.59 | 1285.59 | asthmatic lung | 1.33 |
| asthmatic lung ODO3433 | 29323 | 27.15 | 1145.8 | 2291.60 | asthmatic lung | 2.37 |
| asthmatic lung ODO3397 | 29322 | 26.02 | 2314.97 | 4629.94 | asthmatic lung | 4.78 |
| asthmatic lung ODO4928 | 29325 | 27 | 1256.03 | 2512.06 | asthmatic lung | 2.59 |
| endo cells | control | 32.22 | 49.23 | 49.23 | endo cells | |
| endo VEGF | | 33.05 | 29.29 | 29.29 | endo VEGF | −1.68 |
| endo bFGF | | 32.71 | 36.25 | 36.25 | endo bFGF | −1.36 |
| heart Clontech | normal | 32.81 | 34.06 | 68.12 | heart | |
| heart (T-1) ischemic | 29417 | 31.63 | 70.95 | 141.90 | heart (T-1) | 2.08 |
| | | | | | ischemic | |
| heart (T-14) non- | 29422 | 31.01 | 104.06 | 208.12 | heart (T-14) non- | 3.06 |
| obstructive DCM | | | | | obstructive DCM | |
| heart (T-3399) DCM | 29426 | 29.87 | 211.37 | 422.74 | heart (T-3399) | 6.21 |
| | | | | | DCM | |
| adenoid GW99-269 | 26162 | 30.24 | 167.48 | 334.96 | adenoid | |
| tonsil GW98-280 | 22582 | 27.83 | 751.19 | 1502.38 | tonsil | |
| T cells PC00314 | 28453 | 30.69 | 126.89 | 253.78 | T cells | |
| PBMNC | | 32.29 | 47.02 | 47.02 | PBMNC | |
| monocyte | | 32.31 | 46.34 | 92.68 | monocyte | |
| B cells PC00665 | 28455 | 28.55 | 479.1 | 958.20 | B cells | |
| dendritic cells | 28441 | 27.78 | 773.27 | 1546.54 | dendritic cells | |
| neutrophils | 28440 | 28.34 | 546.2 | 546.20 | neutrophils | |
| eosinophils | 28446 | 29.01 | 361 | 722.00 | eosinophils | |
| BM unstim | | 31.28 | 87.86 | 87.86 | BM unstim | |
| BM stim | treated | 29.46 | 273.53 | 273.53 | BM stim | 3.11 |
| osteo dif | treated | 34.37 | 12.89 | 12.89 | osteo dif | 3.04 |
| osteo undif | | 36.16 | 4.24 | 4.24 | osteo undif | |
| chondrocytes | | 29.72 | 231.78 | 579.45 | chondrocytes | |
| OA Synovium IP12/01 | 29462 | 29.86 | 212.56 | 212.56 | OA Synovium | |
| OA Synovium NP10/01 | 29461 | 31.01 | 103.92 | 207.84 | OA Synovium | |
| OA Synovium NP57/00 | 28464 | 28.77 | 418.09 | 836.18 | OA Synovium | |
| RA Synovium | 28466 | 28.72 | 431.53 | 863.06 | RA Synovium | |
| NP03/01 | | | | | | |
| RA Synovium | 28467 | 29.07 | 347.77 | 695.54 | RA Synovium | |
| NP71/00 | | | | | | |
| RA Synovium | 28475 | 28.56 | 476.95 | 953.90 | RA Synovium | |
| NP45/00 | | | | | | |
| OA bone (biobank) | 29217 | 29.73 | 230.13 | 230.13 | OA bone | |
| | | | | | (biobank) | |
| OA bone Sample 1 | J. Emory | 29.35 | 292.22 | 584.44 | OA bone | |
| OA bone Sample 2 | J. Emory | 28.82 | 406.11 | 812.22 | OA bone | |
| Cartilage (pool) | Normal | 29.46 | 272.55 | 545.10 | Nml Cartilage | |
| | | | | | (pool) | |
| Cartilage (pool) | OA | 30.07 | 186.61 | 373.22 | OA Cartilage | −1.46 |
| | | | | | (pool) | |
| PBL unifected | 28441 | 24.46 | 6094.2 | 12188.40 | PBL unifected | |
| PBL HIV IIIB | 28442 | 27.78 | 773.53 | 1547.06 | PBL HIV IIIB | −7.88 |
| MRC5 uninfected | 29158 | 30.08 | 185.3 | 370.60 | MRC5 uninfected | |
| (100%) | | | | | (100%) | |
| MRC5 HSV strain F | 29178 | 31.26 | 89.14 | 178.28 | MRC5 HSV | −2.08 |
| | | | | | strain F | |
| W12 cells | 29179 | 24.9 | 4626.33 | 9252.66 | W12 cells | 1.29 |
| Keratinocytes | 29180 | 25.31 | 3594.16 | 7188.32 | Keratinocytes | |
| B-actin control | | 27.42 | 967.12 | | | |
| genomic | | 26.69 | 1523.16 | | | |
| 1.00E+05 | | 20.06 | 100000 | | | |
| 1.00E+05 | | 20.16 | 100000 | | | |
| 1.00E+04 | | 23.58 | 10000 | | | |
| 1.00E+04 | | 23.43 | 10000 | | | |
| 1.00E+03 | | 26.98 | 1000 | | | |
| 1.00E+03 | | 27.03 | 1000 | | | |
| 1.00E+02 | | 31.54 | 100 | | | |
| 1.00E+02 | | 31.81 | 100 | | | |
| 1.00E+01 | | 34 | 10 | | | |
| 1.00E+01 | | 35.1 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1536324mapkkk

| | Fold Change in Disease |
|---|---|
| | Population Relative to |
| Disease tissues | Normal |
| colon tumor | −1.26 |
| colon tumor | 39.19 |
| colon tumor | 1.33 |
| colon tumor | 1.56 |
| lung tumor | −1.12 |
| lung tumor | −1.15 |
| lung tumor | −1.16 |
| lung tumor | −3.04 |
| breast tumor | 3.38 |
| breast tumor | 2.42 |
| breast tumor | −2.07 |
| breast tumor | −2.01 |
| brain stage 5 ALZ | −4.31 |
| brain stage 5 ALZ | −1.09 |
| brain stage 5 ALZ | −1.29 |
| brain stage 5 ALZ | 1.09 |
| lung 24 COPD | −7.27 |
| lung 28 COPD | −5.12 |
| lung 23 COPD | −10.14 |
| asthmatic lung | 1.33 |
| asthmatic lung | 2.37 |
| asthmatic lung | 4.78 |
| asthmatic lung | 2.59 |
| endo VEGF | −1.68 |
| endo bFGF | −1.36 |
| heart (T-1) ischemic | 2.08 |
| heart (T-14) non-obstructive DCM | 3.06 |
| heart (T-3399) DCM | 6.21 |
| BM stim | 3.11 |
| osteo dif | 3.04 |
| OA Cartilage (pool) | −1.46 |
| PBL HIV IIIB | −7.88 |
| MRC5 HSV strain F | −2.08 |
| W12 cells | 1.29 |

Gene Name gsk1810944BrCaAg

Moderate to low overall expression. The highest normal expression is seen in the pancreas, prostate, and testis. The highest disease expression is seen in the normal and tumor breast samples. Upregulation in 2 of 4 colon tumors, 2 of 4 lung tumors, and 3 of 4 breast tumors implicates this gene in cancers of the colon, lung, and breast. Upregulation in 3 of 4 Alzheimer's brain samples and downregulation in 1 of 4 Alzheimer's brain samples suggests an involvement in Alzheimer's disease. Downregulated in the stimulated bone marrow. Downregulation in the HIV-infected PBLs suggests that this gene may play a role as a host factor in HIV. Upregulation in the HSV-infected MRC5 cells and the W12 cells suggests that this gene may be a host factor in HSV and HPV. Patterns of expression are very similar to gsk1811484BrCaAg.

| | | | | | | | copies of |
|---|---|---|---|---|---|---|---|
| | | Mean | Mean | | | | mRNA |
| | | GOI | GOI | | | | detected/ |
| Sample | Ct | copies | copies | Average | 18 S | 50 ng/ | 50 ng |
| gsk1810944- | (sample | (sample | (sample | GOI | rRNA | 18 S | total |
| BrCaAg | 1 and 2) | 1) | 2) | Copies | (ng) | rRNA(ng) | RNA |
| Subcutaneous | 36.25, 36.46 | 0.79 | 0.7 | 0.75 | 3.06 | 16.34 | 12.17 |
| Adipocytes Zenbio | | | | | | | |
| Subcutaneous | 40, 40 | 0 | 0 | 0.00 | 0.96 | 52.36 | 0.00 |
| Adipose Zenbio | | | | | | | |
| Adrenal Gland Clontech | 40, 38.83 | 0 | 0.17 | 0.09 | 0.61 | 81.97 | 6.97 |
| Whole Brain Clontech | 31.12, 31.73 | 17.51 | 12.12 | 14.82 | 7.24 | 6.91 | 102.31 |
| Fetal Brain Clontech | 36.67, 40 | 0.62 | 0 | 0.31 | 0.48 | 103.95 | 32.22 |
| Cerebellum Clontech | 36.89, 36.74 | 0.54 | 0.59 | 0.57 | 2.17 | 23.04 | 13.02 |
| Cervix | 34.39, 33.37 | 2.45 | 4.52 | 3.49 | 2.42 | 20.66 | 72.00 |
| Colon | 33.54, 33.99 | 4.08 | 3.11 | 3.60 | 2.71 | 18.45 | 66.33 |
| Endometrium | 35.71, 34.14 | 1.1 | 2.84 | 1.97 | 0.73 | 68.21 | 134.38 |
| Esophagus | 35.79, 33.75 | 1.05 | 3.58 | 2.32 | 1.37 | 36.50 | 84.49 |
| Heart Clontech | 36.93, 40 | 0.53 | 0 | 0.27 | 1.32 | 37.88 | 10.04 |
| Hypothalamus | 40, 40 | 0 | 0 | 0.00 | 0.32 | 155.28 | 0.00 |
| Ileum | 33.8, 35.28 | 3.49 | 1.43 | 2.46 | 2.58 | 19.38 | 47.67 |
| Jejunum | 31.05, 31.01 | 18.19 | 18.69 | 18.44 | 6.60 | 7.58 | 139.70 |
| Kidney | 36.21, 36.17 | 0.82 | 0.84 | 0.83 | 2.12 | 23.58 | 19.58 |
| Liver | 32.55, 32.09 | 7.41 | 9.73 | 8.57 | 1.50 | 33.33 | 285.67 |
| Fetal Liver Clontech | 35.99, 35.95 | 0.93 | 0.95 | 0.94 | 10.40 | 4.81 | 4.52 |
| Lung | 35.03, 34.86 | 1.66 | 1.83 | 1.75 | 2.57 | 19.46 | 33.95 |
| Mammary Gland | 30.57, 30.66 | 24.41 | 23.12 | 23.77 | 13.00 | 3.85 | 91.40 |
| Clontech | | | | | | | |
| Myometrium | 35.15, 36.05 | 1.54 | 0.9 | 1.22 | 2.34 | 21.37 | 26.07 |
| Omentum | 30.24, 29.99 | 29.64 | 34.55 | 32.10 | 3.94 | 12.69 | 407.30 |
| Ovary | 31.21, 31.39 | 16.53 | 14.89 | 15.71 | 4.34 | 11.52 | 180.99 |
| Pancreas | 29.25, 34.15 | 53.86 | 2.82 | 28.34 | 0.81 | 61.80 | 1751.55 |
| Head of Pancreas | 34.95, 35.35 | 1.74 | 1.37 | 1.56 | 1.57 | 31.85 | 49.52 |
| Parotid Gland | 32.25, 33.2 | 8.84 | 5.01 | 6.93 | 5.48 | 9.12 | 63.18 |
| Placenta Clontech | 29.7, 29.86 | 41.05 | 37.31 | 39.18 | 5.26 | 9.51 | 372.43 |
| Prostate | 29.4, 29.21 | 49.3 | 55.13 | 52.22 | 3.00 | 16.67 | 870.25 |
| Rectum | 37.2, 36.09 | 0.45 | 0.87 | 0.66 | 1.23 | 40.65 | 26.83 |
| Salivary Gland Clontech | 33.48, 32.85 | 4.22 | 6.18 | 5.20 | 7.31 | 6.84 | 35.57 |
| Skeletal Muscle Clontech | 40, 40 | 0 | 0 | 0.00 | 1.26 | 39.68 | 0.00 |
| Skin | 35.73, 34.57 | 1.09 | 2.19 | 1.64 | 1.21 | 41.32 | 67.77 |
| Small Intestine Clontech | 40, 40 | 0 | 0 | 0.00 | 0.98 | 51.07 | 0.00 |
| Spleen | 32.81, 32.51 | 6.33 | 7.56 | 6.95 | 4.92 | 10.16 | 70.58 |
| Stomach | 30.42, 30.07 | 26.58 | 32.91 | 29.75 | 2.73 | 18.32 | 544.78 |
| Testis Clontech | 28.85, 28.49 | 68.5 | 85.03 | 76.77 | 0.57 | 87.87 | 6745.61 |
| Thymus Clontech | 28.84, 28.44 | 69.07 | 87.85 | 78.46 | 9.89 | 5.06 | 396.66 |
| Thyroid | 37.78, 37.22 | 0.32 | 0.44 | 0.38 | 2.77 | 18.05 | 6.86 |
| Trachea Clontech | 28.54, 28.05 | 82.88 | 111.3 | 97.09 | 9.71 | 5.15 | 499.95 |
| Urinary Bladder | 35.71, 35.32 | 1.1 | 1.39 | 1.25 | 5.47 | 9.14 | 11.38 |
| Uterus | 30.33, 30.28 | 28.15 | 28.94 | 28.55 | 5.34 | 9.36 | 267.28 |
| genomic | 23.66 | 1559.65 | | | | | |
| b-actin | 27.75 | 133.39 | | | | | |
| 1.00E+05 | 17.56 | 100000 | | | | | |
| 1.00E+05 | 17.6 | 100000 | | | | | |
| 1.00E+04 | 20.22 | 10000 | | | | | |
| 1.00E+04 | 20.22 | 10000 | | | | | |
| 1.00E+03 | 23.66 | 1000 | | | | | |
| 1.00E+03 | 23.5 | 1000 | | | | | |
| 1.00E+02 | 28.44 | 100 | | | | | |
| 1.00E+02 | 27.66 | 100 | | | | | |
| 1.00E+01 | 32.2 | 10 | | | | | |
| 1.00E+01 | 32.08 | 10 | | | | | |
| 1.00E+00 | 40 | 1 | | | | | |
| 1.00E+00 | 36.72 | 1 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| gsk1810944- | (GSK | | GOI | total | | Disease |
| BrCaAg | identifier) | Ct | copies | RNA | Sample | Population |
| colon normal GW98-167 | 21941 | 31.6 | 16.07 | 32.14 | colon normal | |
| colon tumor GW98-166 | 21940 | 26.43 | 401.48 | 802.96 | colon tumor | 24.98 |
| colon normal GW98-178 | 22080 | 35.59 | 1.34 | 2.68 | colon normal | |
| colon tumor GW98-177 | 22060 | 34 | 3.61 | 7.22 | colon tumor | 2.69 |
| colon normal GW98-561 | 23514 | 34.42 | 2.79 | 5.58 | colon normal | |
| colon tumor GW98-560 | 23513 | 34.98 | 1.97 | 3.94 | colon tumor | −1.42 |
| colon normal GW98-894 | 24691 | 32.07 | 12.03 | 24.06 | colon normal | |
| colon tumor GW98-893 | 24690 | 29.59 | 56.38 | 112.76 | colon tumor | 4.69 |
| lung normal GW98-3 | 20742 | 31.62 | 15.94 | 31.88 | lung normal | |
| lung tumor GW98-2 | 20741 | 25.47 | 731.76 | 1463.52 | lung tumor | 45.91 |
| lung normal GW97-179 | 20677 | 28.71 | 97.31 | 194.62 | lung normal | |
| lung tumor GW97-178 | 20676 | 31.26 | 19.96 | 39.92 | lung tumor | −4.88 |
| lung normal GW98-165 | 21922 | 31.38 | 18.43 | 36.86 | lung normal | |
| lung tumor GW98-164 | 21921 | 28.19 | 134.24 | 268.48 | lung tumor | 7.28 |
| lung normal GW98-282 | 22584 | 30.18 | 38.92 | 77.84 | lung normal | |
| lung tumor GW98-281 | 22583 | 34.33 | 2.95 | 5.90 | lung tumor | −13.19 |
| breast normal GW00-392 | 28750 | 22.69 | 4112.84 | 4112.84 | breast normal | |
| breast tumor GW00-391 | 28746 | 24.07 | 1742.01 | 3484.02 | breast tumor | −1.18 |
| breast normal GW00-413 | 28798 | 25.17 | 879.08 | 879.08 | breast normal | |
| breast tumor GW00-412 | 28797 | 22.83 | 3790.96 | 7581.92 | breast tumor | 8.62 |
| breast normal GW00- | 27592-95 | 27.9 | 160.94 | 160.94 | breast normal | |
| 235:238 | | | | | | |
| breast tumor GW00- | 27588-91 | 22.45 | 4774.85 | 4774.85 | breast tumor | 29.67 |
| 231:234 | | | | | | |
| breast normal GW98-621 | 23656 | 26.47 | 392.57 | 785.14 | breast normal | |
| breast tumor GW98-620 | 23655 | 24.07 | 1744.99 | 3489.98 | breast tumor | 4.45 |
| brain normal BB99-542 | 25507 | 36.64 | 0.7 | 1.40 | brain normal | |
| brain normal BB99-406 | 25509 | 34.99 | 1.95 | 3.90 | brain normal | |
| brain normal BB99-904 | 25546 | 35.36 | 1.55 | 3.10 | brain normal | |
| brain stage 5 ALZ BB99- | 25502 | 39.15 | 0.15 | 0.30 | brain stage 5 ALZ | −9.33 |
| 874 | | | | | | |
| brain stage 5 ALZ BB99- | 25503 | 32.36 | 10.05 | 20.10 | brain stage 5 ALZ | 7.18 |
| 887 | | | | | | |
| brain stage 5 ALZ BB99- | 25504 | 33.44 | 5.14 | 10.28 | brain stage 5 ALZ | 3.67 |
| 862 | | | | | | |
| brain stage 5 ALZ BB99- | 25542 | 32.86 | 7.35 | 14.70 | brain stage 5 ALZ | 5.25 |
| 927 | | | | | | |
| CT lung | normal | 33.56 | 4.76 | 9.52 | CT lung Nml | |
| lung 26 | normal | 34.06 | 3.49 | | lung 26 Nml | |
| lung 27 | normal | 37.17 | 0.5 | 0.50 | lung 27 Nml | |
| lung 24 | COPD | 36.1 | 0.98 | 0.98 | lung 24 COPD | −3.56 |
| lung 28 | COPD | 39.98 | 0.07 | 0.07 | lung 28 COPD | −49.90 |
| lung 23 | COPD | 35.14 | 1.78 | 1.78 | lung 23 COPD | −1.96 |
| lung 25 | normal | 37.32 | 0.46 | 0.46 | lung 25 Nml | |
| asthmatic lung ODO3112 | 29321 | 36.93 | 0.58 | 0.58 | asthmatic lung | −6.02 |
| asthmatic lung ODO3433 | 29323 | 36.24 | 0.9 | 1.80 | asthmatic lung | −1.94 |
| asthmatic lung ODO3397 | 29322 | 33.42 | 5.2 | 10.40 | asthmatic lung | 2.98 |
| asthmatic lung ODO4928 | 29325 | 33.65 | 4.49 | 8.98 | asthmatic lung | 2.57 |
| endo cells | control | 40 | 0 | 0.00 | endo cells | |
| endo VEGF | | 38.26 | 0.26 | 0.26 | endo VEGF | 0.26 |
| endo bFGF | | 35.43 | 1.48 | 1.48 | endo bFGF | 1.48 |
| heart Clontech | normal | 40 | 0 | 0.00 | heart | |
| heart (T-1) ischemic | 29417 | 40 | 0 | 0.00 | heart (T-1) | 0.00 |
| | | | | | ischemic | |
| heart (T-14) non- | 29422 | 37.97 | 0.31 | 0.62 | heart (T-14) non- | 0.62 |
| obstructive DCM | | | | | obstructive DCM | |
| heart (T-3399) DCM | 29426 | 37.3 | 0.47 | 0.94 | heart (T-3399) | 0.94 |
| | | | | | DCM | |
| adenoid GW99-269 | 26162 | 32.94 | 7.02 | 14.04 | adenoid | |
| tonsil GW98-280 | 22582 | 31.93 | 13.09 | 26.18 | tonsil | |
| T cells PC00314 | 28453 | 33.8 | 4.09 | 8.18 | T cells | |
| PBMNC | | 38.2 | 0.26 | 0.26 | PBMNC | |
| monocyte | | 37.25 | 0.48 | 0.96 | monocyte | |
| B cells PC00665 | 28455 | 34.27 | 3.06 | 6.12 | B cells | |
| dendritic cells | 28441 | 36.7 | 0.67 | 1.34 | dendritic cells | |
| neutrophils | 28440 | 33.4 | 5.25 | 5.25 | neutrophils | |
| eosinophils | 28446 | 37.21 | 0.49 | 0.98 | eosinophils | |
| BM unstim | | 35.01 | 1.93 | 1.93 | BM unstim | |
| BM stim | treated | 38.15 | 0.27 | 0.27 | BM stim | −7.15 |
| osteo dif | treated | 40 | 0 | 0.00 | osteo dif | 0.00 |
| osteo undif | | 40 | 0 | 0.00 | osteo undif | |
| chondrocytes | | 35.2 | 1.72 | 4.30 | chondrocytes | |
| OA Synovium IP12/01 | 29462 | 37.23 | 0.49 | 0.49 | OA Synovium | |
| OA Synovium NP10/01 | 29461 | 35.26 | 1.65 | 3.30 | OA Synovium | |
| OA Synovium NP57/00 | 28464 | 34.19 | 3.22 | 6.44 | OA Synovium | |
| RA Synovium | 28466 | 36.43 | 0.8 | 1.60 | RA Synovium | |
| NP03/01 | | | | | | |
| RA Synovium | 28467 | 35.53 | 1.4 | 2.80 | RA Synovium | |
| NP71/00 | | | | | | |
| RA Synovium | 28475 | 34.14 | 3.32 | 6.64 | RA Synovium | |
| NP45/00 | | | | | | |
| OA bone (biobank) | 29217 | 36.22 | 0.91 | 0.91 | OA bone | |
| | | | | | (biobank) | |
| OA bone Sample 1 | J. Emory | 34.28 | 3.05 | 6.10 | OA bone | |
| OA bone Sample 2 | J. Emory | 34.09 | 3.42 | 6.84 | OA bone | |
| Cartilage (pool) | Normal | 35.74 | 1.23 | 2.46 | Nml Cartilage | |
| | | | | | (pool) | |
| Cartilage (pool) | OA | 34.69 | 2.36 | 4.72 | OA Cartilage | 1.92 |
| | | | | | (pool) | |
| PBL unifected | 28441 | 35.36 | 1.55 | 3.10 | PBL unifected | |
| PBL HIV IIIB | 28442 | 39.79 | 0.1 | 0.20 | PBL HIV IIIB | −15.50 |
| MRC5 uninfected | 29158 | 37.66 | 0.37 | 0.74 | MRC5 uninfected | |
| (100%) | | | | | (100%) | |
| MRC5 HSV strain F | 29178 | 30.9 | 24.88 | 49.76 | MRC5 HSV | 67.24 |
| | | | | | strain F | |
| W12 cells | 29179 | 33.33 | 5.48 | 10.96 | W12 cells | 5.27 |
| Keratinocytes | 29180 | 36.01 | 1.04 | 2.08 | Keratinocytes | |
| B-actin control | | 27.27 | 238.18 | | | |
| genomic | | 24.3 | 1512.29 | | | |
| 1.00E+05 | | 17.99 | 100000 | | | |
| 1.00E+05 | | 17.88 | 100000 | | | |
| 1.00E+04 | | 20.91 | 10000 | | | |
| 1.00E+04 | | 20.93 | 10000 | | | |
| 1.00E+03 | | 24.55 | 1000 | | | |
| 1.00E+03 | | 24.53 | 1000 | | | |
| 1.00E+02 | | 28.88 | 100 | | | |
| 1.00E+02 | | 29.32 | 100 | | | |
| 1.00E+01 | | 32.52 | 10 | | | |
| 1.00E+01 | | 32.17 | 10 | | | |
| 1.00E+00 | | 40. | 0 | | | |
| 1.00E+00 | | 40 | 0 | | | |
| NTC | | 40 | 0 | | | |

*lung 26 Normal has been omitted due to multiple amplification failures from that sample

Gene Name gsk1810944BrCaAg

| | Fold Change in Disease |
|---|---|
| | Population Relative to |
| Disease tissues | Normal |
| colon tumor | 24.98 |
| colon tumor | 2.69 |
| colon tumor | −1.42 |
| colon tumor | 4.69 |
| lung tumor | 45.91 |
| lung tumor | −4.88 |
| lung tumor | 7.28 |
| lung tumor | −13.19 |
| breast tumor | −1.18 |
| breast tumor | 8.62 |
| breast tumor | 29.67 |
| breast tumor | 4.45 |
| brain stage 5 ALZ | −9.33 |
| brain stage 5 ALZ | 7.18 |
| brain stage 5 ALZ | 3.67 |
| brain stage 5 ALZ | 5.25 |
| lung 24 COPD | −3.56 |
| lung 28 COPD | −49.90 |
| lung 23 COPD | −1.96 |
| asthmatic lung | −6.02 |
| asthmatic lung | −1.94 |
| asthmatic lung | 2.98 |
| asthmatic lung | 2.57 |
| endo VEGF | 0.26 |
| endo bFGF | 1.48 |
| heart (T-1) ischemic | 0.00 |
| heart (T-14) non-obstructive DCM | 0.62 |
| heart (T-3399) DCM | 0.94 |
| BM stim | −7.15 |
| osteo dif | 0.00 |
| OA Cartilage (pool) | 1.92 |
| PBL HIV IIIB | −15.50 |
| MRC5 HSV strain F | 67.24 |
| W12 cells | 5.27 |

Gene Name sbg1535914AIP

Moderate to high overall expression. The highest normal expression is seen in the whole brain, fetal liver, and thymus. Expression in the disease samples is fairly ubiquitous with the highest levels seen in the uninfected PBLs, one of the normal brain samples, and one of the Alzheimer's brain samples. Upregulation in 3 of 4 asthmatic lung samples with corroborating high expression in the eosinophils and neutrophils indicates a possible involvement in asthma. Upregulation in 2 of 3 disease heart samples implies an involvement in cardiovascular diseases such as ischemic and obstructive DCM. Upregulated in the differentiated osteoblasts. Downregulation in the HSV-infected MRC5 cells suggests that this gene may be a host factor in HSV. High expression in the RA and OA synovium samples, the OA bone samples, and the chondrocytes with corroborating high expression in all of the inflammatory immune cells implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample | (sample | (sample | GOI | rRNA | 18 S | total |
| sbg1535914AIP | 1 and 2) | 1) | 2) | Copies | (ng) | rRNA(ng) | RNA |
| Subcutaneous | 30.81, 30.46 | 112.89 | 140.5 | 126.70 | 3.06 | 16.34 | 2070.18 |
| Adipocytes Zenbio | | | | | | | |
| Subcutaneous Adipose | 34.7, 34.39 | 10.24 | 12.37 | 11.31 | 0.96 | 52.36 | 591.88 |
| Zenbio | | | | | | | |
| Adrenal Gland | 33.84, 39.7 | 17.46 | 0.47 | 8.97 | 0.61 | 81.97 | 734.84 |
| Clontech | | | | | | | |
| Whole Brain Clontech | 22.13, 21.9 | 23919.64 | 27498.63 | 25709.14 | 7.24 | 6.91 | 177549.27 |
| Fetal Brain Clontech | 34.87, 35.44 | 9.21 | 6.49 | 7.85 | 0.48 | 103.95 | 816.01 |
| Cerebellum Clontech | 32.26, 32.26 | 46.02 | 46.07 | 46.05 | 2.17 | 23.04 | 1060.94 |
| Cervix | 33.36, 32.73 | 23.45 | 34.53 | 28.99 | 2.42 | 20.66 | 598.97 |
| Colon | 30.22, 30.3 | 162.78 | 154.21 | 158.50 | 2.71 | 18.45 | 2924.26 |
| Endometrium | 32.7, 32.66 | 35.11 | 36.06 | 35.59 | 0.73 | 68.21 | 2427.35 |
| Esophagus | 32.26, 32 | 46.26 | 54.24 | 50.25 | 1.37 | 36.50 | 1833.94 |
| Heart Clontech | 35.92, 34.35 | 4.83 | 12.73 | 8.78 | 1.32 | 37.88 | 332.58 |
| Hypothalamus | 34.73, 40 | 10.03 | 0 | 5.02 | 0.32 | 155.28 | 778.73 |
| Ileum | 31.88, 31.15 | 58.17 | 91.33 | 74.75 | 2.58 | 19.38 | 1448.64 |
| Jejunum | 29.25, 28.99 | 295.2 | 347.68 | 321.44 | 6.60 | 7.58 | 2435.15 |
| Kidney | 32.24, 32.22 | 46.8 | 47.39 | 47.10 | 2.12 | 23.58 | 1110.73 |
| Liver | 33.18, 33.58 | 26.14 | 20.44 | 23.29 | 1.50 | 33.33 | 776.33 |
| Fetal Liver Clontech | 24.96, 24.96 | 4158.98 | 4176.02 | 4167.50 | 10.40 | 4.81 | 20036.06 |
| Lung | 31.98, 31.97 | 54.98 | 55.28 | 55.13 | 2.57 | 19.46 | 1072.57 |
| Mammary Gland | 28.42, 28.17 | 494.03 | 574.4 | 534.22 | 13.00 | 3.85 | 2054.67 |
| Clontech | | | | | | | |
| Myometrium | 31.79, 31.76 | 61.72 | 62.76 | 62.24 | 2.34 | 21.37 | 1329.91 |
| Omentum | 33.01, 32.19 | 28.97 | 48.27 | 38.62 | 3.94 | 12.69 | 490.10 |
| Ovary | 31.49, 31.72 | 74.23 | 64.52 | 69.38 | 4.34 | 11.52 | 799.25 |
| Pancreas | 34.55, 34.36 | 11.23 | 12.65 | 11.94 | 0.81 | 61.80 | 737.95 |
| Head of Pancreas | 34.7, 35.44 | 10.23 | 6.48 | 8.36 | 1.57 | 31.85 | 266.08 |
| Parotid Gland | 30.48, 30.27 | 138.5 | 157.39 | 147.95 | 5.48 | 9.12 | 1349.86 |
| Placenta Clontech | 30.65, 30.72 | 124.37 | 119.61 | 121.99 | 5.26 | 9.51 | 1159.60 |
| Prostate | 31.23, 31.26 | 86.97 | 85.43 | 86.20 | 3.00 | 16.67 | 1436.67 |
| Rectum | 31.27, 30.53 | 85.15 | 134.2 | 109.68 | 1.23 | 40.65 | 4458.33 |
| Salivary Gland | 29.47, 29.11 | 258.68 | 321.3 | 289.99 | 7.31 | 6.84 | 1983.52 |
| Clontech | | | | | | | |
| Skeletal Muscle | 38.36, 35.39 | 1.07 | 6.68 | 3.88 | 1.26 | 39.68 | 153.77 |
| Clontech | | | | | | | |
| Skin | 35.03, 34.6 | 8.33 | 10.91 | 9.62 | 1.21 | 41.32 | 397.52 |
| Small Intestine | 34.63, 35.35 | 10.71 | 6.84 | 8.78 | 0.98 | 51.07 | 448.16 |
| Clontech | | | | | | | |
| Spleen | 30.44, 30.13 | 141.71 | 171.93 | 156.82 | 4.92 | 10.16 | 1593.70 |
| Stomach | 32.74, 32.96 | 34.32 | 30.02 | 32.17 | 2.73 | 18.32 | 589.19 |
| Testis Clontech | 33.26, 33.73 | 24.93 | 18.6 | 21.77 | 0.57 | 87.87 | 1912.57 |
| Thymus Clontech | 25.09, 24.91 | 3849.53 | 4315.09 | 4082.31 | 9.89 | 5.06 | 20638.57 |
| Thyroid | 31.29, 31.03 | 83.71 | 98.77 | 91.24 | 2.77 | 18.05 | 1646.93 |
| Trachea Clontech | 29.42, 29.01 | 266.68 | 342.37 | 304.53 | 9.71 | 5.15 | 1568.10 |
| Urinary Bladder | 29.92, 29.78 | 195.24 | 213.49 | 204.37 | 5.47 | 9.14 | 1868.05 |
| Uterus | 31.03, 30.71 | 98.49 | 120.24 | 109.37 | 5.34 | 9.36 | 1024.02 |
| genomic | 26.39 | 1729.8 | | | | | |
| b-actin | 27.81 | 720.39 | | | | | |
| 1.00E+05 | 20.29 | 100000 | | | | | |
| 1.00E+05 | 20.08 | 100000 | | | | | |
| 1.00E+04 | 23.44 | 10000 | | | | | |
| 1.00E+04 | 23.43 | 10000 | | | | | |
| 1.00E+03 | 26.59 | 1000 | | | | | |
| 1.00E+03 | 26.94 | 1000 | | | | | |
| 1.00E+02 | 31.39 | 100 | | | | | |
| 1.00E+02 | 30.83 | 100 | | | | | |
| 1.00E+01 | 34.04 | 10 | | | | | |
| 1.00E+01 | 35.19 | 10 | | | | | |
| 1.00E+00 | 38.99 | 1 | | | | | |
| 1.00E+00 | 40 | 1 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1535914AIP | identifier) | Ct | copies | RNA | Sample | Population |
| colon normal GW98-167 | 21941 | 23.65 | 6636.9 | 13273.80 | colon normal | |
| colon tumor GW98-166 | 21940 | 23.51 | 7202.72 | 14405.44 | colon tumor | 1.09 |
| colon normal GW98-178 | 22080 | 26.63 | 1154.59 | 2309.18 | colon normal | |
| colon tumor GW98-177 | 22060 | 25.6 | 2111.16 | 4222.32 | colon tumor | 1.83 |
| colon normal GW98-561 | 23514 | 24.39 | 4304.52 | 8609.04 | colon normal | |
| colon tumor GW98-560 | 23513 | 24.64 | 3700.38 | 7400.76 | colon tumor | −1.16 |
| colon normal GW98-894 | 24691 | 23.8 | 6076.66 | 12153.32 | colon normal | |
| colon tumor GW98-893 | 24690 | 24.02 | 5331.09 | 10662.18 | colon tumor | −1.14 |
| lung normal GW98-3 | 20742 | 24.61 | 3765.05 | 7530.10 | lung normal | |
| lung tumor GW98-2 | 20741 | 26.54 | 1217.31 | 2434.62 | lung tumor | −3.09 |
| lung normal GW97-179 | 20677 | 24.11 | 5059.71 | 10119.42 | lung normal | |
| lung tumor GW97-178 | 20676 | 24.58 | 3839.48 | 7678.96 | lung tumor | −1.32 |
| lung normal GW98-165 | 21922 | 24.23 | 4729.71 | 9459.42 | lung normal | |
| lung tumor GW98-164 | 21921 | 23.91 | 5676.27 | 11352.54 | lung tumor | 1.20 |
| lung normal GW98-282 | 22584 | 24.17 | 4899.41 | 9798.82 | lung normal | |
| lung tumor GW98-281 | 22583 | 25.48 | 2261.81 | 4523.62 | lung tumor | −2.17 |
| breast normal GW00-392 | 28750 | 25.28 | 2554.11 | 2554.11 | breast normal | |
| breast tumor GW00-391 | 28746 | 25.2 | 2670.11 | 5340.22 | breast tumor | 2.09 |
| breast normal GW00-413 | 28798 | 25.7 | 1989.13 | 1989.13 | breast normal | |
| breast tumor GW00-412 | 28797 | 24.78 | 3421.72 | 6843.44 | breast tumor | 3.44 |
| breast normal GW00- | 27592-95 | 27.03 | 909.61 | 909.61 | breast normal | |
| 235:238 | | | | | | |
| breast tumor GW00- | 27588-91 | 25.34 | 2458.97 | 2458.97 | breast tumor | 2.70 |
| 231:234 | | | | | | |
| breast normal GW98-621 | 23656 | 24.85 | 3281.35 | 6562.70 | breast normal | |
| breast tumor GW98-620 | 23655 | 25.2 | 2663.34 | 5326.68 | breast tumor | −1.23 |
| brain normal BB99-542 | 25507 | 24.33 | 4456.24 | 8912.48 | brain normal | |
| brain normal BB99-406 | 25509 | 22.86 | 10547.39 | 21094.78 | brain normal | |
| brain normal BB99-904 | 25546 | 24.16 | 4920.19 | 9840.38 | brain normal | |
| brain stage 5 ALZ BB99- | 25502 | 25.54 | 2180.95 | 4361.90 | brain stage 5 ALZ | −3.05 |
| 874 | | | | | | |
| brain stage 5 ALZ BB99- | 25503 | 22.63 | 12046.47 | 24092.94 | brain stage 5 ALZ | 1.81 |
| 887 | | | | | | |
| brain stage 5 ALZ BB99- | 25504 | 23.97 | 5500.12 | 11000.24 | brain stage 5 ALZ | −1.21 |
| 862 | | | | | | |
| brain stage 5 ALZ BB99- | 25542 | 24.35 | 4400.1 | 8800.20 | brain stage 5 ALZ | −1.51 |
| 927 | | | | | | |
| CT lung | normal | 25.14 | 2760.31 | 5520.62 | CT lung Nml | |
| lung 26 | normal | 30.29 | 134.92 | | lung 26 Nml | |
| lung 27 | normal | 31.37 | 71.48 | 71.48 | lung 27 Nml | |
| lung 24 | COPD | 31.17 | 80.44 | 80.44 | lung 24 COPD | −23.71 |
| lung 28 | COPD | 29.86 | 173.39 | 173.39 | lung 28 COPD | −11.00 |
| lung 23 | COPD | 30.33 | 131.84 | 131.84 | lung 23 COPD | −14.47 |
| lung 25 | normal | 30.35 | 130.26 | 130.26 | lung 25 Nml | |
| asthmatic lung ODO3112 | 29321 | 25.53 | 2200.74 | 2200.74 | asthmatic lung | 1.15 |
| asthmatic lung ODO3433 | 29323 | 25.45 | 2303.5 | 4607.00 | asthmatic lung | 2.42 |
| asthmatic lung ODO3397 | 29322 | 24.16 | 4919 | 9838.00 | asthmatic lung | 5.16 |
| asthmatic lung ODO4928 | 29325 | 24.81 | 3362.05 | 6724.10 | asthmatic lung | 3.53 |
| endo cells | control | 27.95 | 532.18 | 532.18 | endo cells | |
| endo VEGF | | 28.9 | 303.89 | 303.89 | endo VEGF | −1.75 |
| endo bFGF | | 28.09 | 489.39 | 489.39 | endo bFGF | −1.09 |
| heart Clontech | normal | 27.46 | 710.71 | 1421.42 | heart | |
| heart (T-1) ischemic | 29417 | 25.3 | 2516.82 | 5033.64 | h art (T-1) | 3.54 |
| | | | | | ischemic | |
| heart (T-14) non- | 29422 | 26.44 | 1289.17 | 2578.34 | heart (T-14) non- | 1.81 |
| obstructive DCM | | | | | obstructive DCM | |
| heart (T-3399) DCM | 29426 | 25.73 | 1950.41 | 3900.82 | heart (T-3399) | 2.74 |
| | | | | | DCM | |
| adenoid GW99-269 | 26162 | 26.09 | 1583.81 | 3167.62 | adenoid | |
| tonsil GW98-280 | 22582 | 24.66 | 3655.92 | 7311.84 | tonsil | |
| T cells PC00314 | 28453 | 23.86 | 5864.93 | 11729.86 | T cells | |
| PBMNC | | 29.51 | 213.37 | 213.37 | PBMNC | |
| monocyte | | 28.58 | 366.84 | 733.68 | monocyte | |
| B cells PC00665 | 28455 | 25.43 | 2329.91 | 4659.82 | B cells | |
| dendritic cells | 28441 | 23.67 | 6572.31 | 13144.62 | dendritic cells | |
| neutrophils | 28440 | 22.21 | 15459.9 | 15459.90 | neutrophils | |
| eosinophils | 28446 | 23.3 | 8144.32 | 16288.64 | eosinophils | |
| BM unstim | | 26.68 | 1121.85 | 1121.85 | BM unstim | |
| BM stim | treated | 26.61 | 1164.69 | 1164.69 | BM stim | 1.04 |
| osteo dif | treated | 27.71 | 613.14 | 613.14 | osteo dif | 4.78 |
| osteo undif | | 30.37 | 128.24 | 128.24 | osteo undif | |
| chondrocytes | | 24.62 | 3743.39 | 9358.48 | chondrocytes | |
| OA Synovium IP12/01 | 29462 | 25.4 | 2369.7 | 2369.70 | OA Synovium | |
| OA Synovium NP10/01 | 29461 | 26.02 | 1653.82 | 3307.64 | OA Synovium | |
| OA Synovium NP57/00 | 28464 | 24.91 | 3165.35 | 6330.70 | OA Synovium | |
| RA Synovium | 28466 | 24.21 | 4763.55 | 9527.10 | RA Synovium | |
| NP03/01 | | | | | | |
| RA Synovium | 28467 | 24.4 | 4261.99 | 8523.98 | RA Synovium | |
| NP71/00 | | | | | | |
| RA Synovium | 28475 | 24.49 | 4048.08 | 8096.16 | RA Synovium | |
| NP45/00 | | | | | | |
| OA bone (biobank) | 29217 | 24.67 | 3647.85 | 3647.85 | OA bone (biobank) | |
| OA bone Sample 1 | J. Emory | 25.41 | 2364.62 | 4729.24 | OA bone | |
| OA bone Sample 2 | J. Emory | 24.34 | 4428.02 | 8856.04 | OA bone | |
| Cartilage (pool) | Normal | 25.42 | 2344.99 | 4689.98 | Nml Cartilage | |
| | | | | | (pool) | |
| Cartilage (pool) | OA | 26.46 | 1276.84 | 2553.68 | OA Cartilage (pool) | −1.84 |
| PBL unifected | 28441 | 22.06 | 16867.39 | 33734.78 | PBL unifected | |
| PBL HIV IIIB | 28442 | 23.23 | 8471.06 | 16942.12 | PBL HIV IIIB | −1.99 |
| MRC5 uninfected | 29158 | 24.83 | 3317.63 | 6635.26 | MRC5 uninfected | |
| (100%) | | | | | (100%) | |
| MRC5 HSV strain F | 29178 | 29.28 | 243.95 | 487.90 | MRC5 HSV strain | −13.60 |
| | | | | | F | |
| W12 cells | 29179 | 24.53 | 3954.56 | 7909.12 | W12 cells | −1.02 |
| Keratinocytes | 29180 | 24.5 | 4023.91 | 8047.82 | Keratinocytes | |
| B-actin control | | 27.54 | 675.35 | | | |
| genomic | | 26.17 | 1514.95 | | | |
| 1.00E+05 | | 19.51 | 100000 | | | |
| 1.00E+05 | | 19.62 | 100000 | | | |
| 1.00E+04 | | 22.92 | 10000 | | | |
| 1.00E+04 | | 22.94 | 10000 | | | |
| 1.00E+03 | | 26.46 | 1000 | | | |
| 1.00E+03 | | 26.25 | 1000 | | | |
| 1.00E+02 | | 30.5 | 100 | | | |
| 1.00E+02 | | 30.23 | 100 | | | |
| 1.00E+01 | | 34.13 | 10 | | | |
| 1.00E+01 | | 34.91 | 10 | | | |
| 1.00E+00 | | 40 | 0 | | | |
| 1.00E+00 | | 39.9 | 1 | | | |
| NTC | | 40 | 0 | | | |

*lung 26 Normal has been omitted due to multiple amplification failures from that sample

Gene Name sbg1535914AIP

| | Fold Change in Disease |
|---|---|
| | Population Relative to |
| Disease tissues | Normal |
| colon tumor | 1.09 |
| colon tumor | 1.83 |
| colon tumor | −1.16 |
| colon tumor | −1.14 |
| lung tumor | −3.09 |
| lung tumor | −1.32 |
| lung tumor | 1.20 |
| lung tumor | −2.17 |
| breast tumor | 2.09 |
| breast tumor | 3.44 |
| breast tumor | 2.70 |
| breast tumor | −1.23 |
| brain stage 5 ALZ | −3.05 |
| brain stage 5 ALZ | 1.81 |
| brain stage 5 ALZ | −1.21 |
| brain stage 5 ALZ | −1.51 |
| lung 24 COPD | −23.71 |
| lung 28 COPD | −11.00 |
| lung 23 COPD | −14.47 |
| asthmatic lung | 1.15 |
| asthmatic lung | 2.42 |
| asthmatic lung | 5.16 |
| asthmatic lung | 3.53 |
| endo VEGF | −1.75 |
| endo bFGF | −1.09 |
| heart (T-1) ischemic | 3.54 |
| heart (T-14) non-obstructive DCM | 1.81 |
| heart (T-3399) DCM | 2.74 |
| BM stim | 1.04 |
| osteo dif | 4.78 |
| OA Cartilage (pool) | −1.84 |
| PBL HIV IIIB | −1.99 |
| MRC5 HSV strain F | −13.60 |
| W12 cells | −1.02 |

Gene Name sbg1530894aCE

Moderate overall expression. The highest normal expression is seen in the whole brain, fetal brain, testis, liver, omentum, parotid gland, and ovary. The highest disease expression is seen in the Alzheimer's disease brain samples. Downregulation in the VEGF-treated endothelial cells suggests that this gene may be involved in angiogenesis. Upregulation in 2 of 3 disease heart samples implies an involvement in cardiovascular diseases such as obstructive and non-obstructive DCM. Upregulated in the stimulated bone marrow. Upregulation in the HIV-infected PBLs and the W12 cells suggests that this gene may be a host factor in HIV and HPV. Moderate to high expression in the RA and OA synovium samples, the OA bone samples, and the chondrocytes with corroborating high expression in the T cells and B cells implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample | (sample | (sample | GOI | rRNA | 18 S | total |
| sbg1530894aCE | 1 and 2) | 1) | 2) | Copies | (ng) | rRNA(ng) | RNA |
| Subcutaneous | 40, 37.11 | 0 | 1.86 | 0.93 | 3.06 | 16.34 | 15.20 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 29.07, 29 | 258.76 | 270.76 | 264.76 | 7.24 | 6.91 | 1828.45 |
| Fetal Brain Clontech | 34.98, 33.62 | 6.87 | 15.79 | 11.33 | 0.48 | 103.95 | 1177.75 |
| Cerebellum Clontech | 32.97, 34.18 | 23.6 | 11.22 | 17.41 | 2.17 | 23.04 | 401.15 |
| Cervix | 34.6, 36.44 | 8.66 | 2.79 | 5.73 | 2.42 | 20.66 | 118.29 |
| Colon | 34.11, 34.54 | 11.69 | 8.96 | 10.33 | 2.71 | 18.45 | 190.50 |
| Endometrium | 33.9, 40 | 13.33 | 0 | 6.67 | 0.73 | 68.21 | 454.64 |
| Esophagus | 36.21, 36.12 | 3.22 | 3.41 | 3.32 | 1.37 | 36.50 | 120.99 |
| Heart Clontech | 40, 40 | 0 | 0 | 0.00 | 1.32 | 37.88 | 0.00 |
| Hypothalamus | 36.09, 40 | 3.46 | 0 | 1.73 | 0.32 | 155.28 | 268.63 |
| Ileum | 36.82, 35.49 | 2.22 | 5.02 | 3.62 | 2.58 | 19.38 | 70.16 |
| Jejunum | 32.47, 32.5 | 32 | 31.38 | 31.69 | 6.60 | 7.58 | 240.08 |
| Kidney | 36.29, 40 | 3.06 | 0 | 1.53 | 2.12 | 23.58 | 36.08 |
| Liver | 32.38, 32.77 | 33.92 | 26.57 | 30.25 | 1.50 | 33.33 | 1008.17 |
| Fetal Liver Clontech | 37.95, 37.39 | 1.11 | 1.56 | 1.34 | 10.40 | 4.81 | 6.42 |
| Lung | 36.12, 35.59 | 3.39 | 4.73 | 4.06 | 2.57 | 19.46 | 78.99 |
| Mammary Gland | 34.03, 33.25 | 12.27 | 19.84 | 16.06 | 13.00 | 3.85 | 61.75 |
| Clontech | | | | | | | |
| Myometrium | 35.09, 34.77 | 6.41 | 7.79 | 7.10 | 2.34 | 21.37 | 151.71 |
| Omentum | 31.81, 31.91 | 47.94 | 45.24 | 46.59 | 3.94 | 12.69 | 591.24 |
| Ovary | 31.03, 31.75 | 77.48 | 49.85 | 63.67 | 4.34 | 11.52 | 733.47 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 31.42, 31.11 | 60.97 | 73.66 | 67.32 | 5.48 | 9.12 | 614.19 |
| Placenta Clontech | 35.03, 35.02 | 6.64 | 6.69 | 6.67 | 5.26 | 9.51 | 63.36 |
| Prostate | 35.1, 34.64 | 6.38 | 8.46 | 7.42 | 3.00 | 16.67 | 123.67 |
| Rectum | 35.71, 40 | 4.39 | 0.57 | 2.48 | 1.23 | 40.65 | 100.81 |
| Salivary Gland Clontech | 36.34, 38.19 | 2.98 | 0.95 | 1.97 | 7.31 | 6.84 | 13.44 |
| Skeletal Muscle Clontech | 40, 40 | 0 | 0 | 0.00 | 1.26 | 39.68 | 0.00 |
| Skin | 36.02, 34.31 | 3.61 | 10.32 | 6.97 | 1.21 | 41.32 | 287.81 |
| Small Intestine Clontech | 40, 40 | 0.15 | 0 | 0.08 | 0.98 | 51.07 | 3.83 |
| Spleen | 33.71, 34.31 | 14.95 | 10.32 | 12.64 | 4.92 | 10.16 | 128.40 |
| Stomach | 34.6, 34.41 | 8.68 | 9.75 | 9.22 | 2.73 | 18.32 | 168.77 |
| Testis Clontech | 34.24, 33.46 | 10.78 | 17.47 | 14.13 | 0.57 | 87.87 | 1241.21 |
| Thymus Clontech | 33.57, 33.09 | 16.35 | 21.95 | 19.15 | 9.89 | 5.06 | 96.81 |
| Thyroid | 38.55, 36.89 | 0.77 | 2.12 | 1.45 | 2.77 | 18.05 | 26.08 |
| Trachea Clontech | 33.11, 33.28 | 21.65 | 19.47 | 20.56 | 9.71 | 5.15 | 105.87 |
| Urinary Bladder | 35.33, 36.01 | 5.51 | 3.63 | 4.57 | 5.47 | 9.14 | 41.77 |
| Uterus | 32.12, 31.6 | 39.63 | 54.81 | 47.22 | 5.34 | 9.36 | 442.13 |
| genomic | 26.53 | 1234.26 | | | | | |
| b-actin | 27.46 | 694.34 | | | | | |
| 1.00E+05 | 18.91 | 100000 | | | | | |
| 1.00E+05 | 19.67 | 100000 | | | | | |
| 1.00E+04 | 23.04 | 10000 | | | | | |
| 1.00E+04 | 23.14 | 10000 | | | | | |
| 1.00E+03 | 26.9 | 1000 | | | | | |
| 1.00E+03 | 26.66 | 1000 | | | | | |
| 1.00E+02 | 30.73 | 100 | | | | | |
| 1.00E+02 | 31.68 | 100 | | | | | |
| 1.00E+01 | 33.59 | 10 | | | | | |
| 1.00E+01 | 40 | 0 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1530894aCE | identifier) | Ct | copies | RNA | Sample | Population |
| colon normal GW98-167 | 21941 | 32.47 | 43.02 | 86.05 | colon normal | |
| colon tumor GW98-166 | 21940 | 35.38 | 10.86 | 21.73 | colon tumor | −3.96 |
| colon normal GW98-178 | 22080 | 35.51 | 10.24 | 20.49 | colon normal | |
| colon tumor GW98-177 | 22060 | 36.12 | 7.79 | 15.59 | colon tumor | −1.31 |
| colon normal GW98-561 | 23514 | 34.78 | 14.29 | 28.58 | colon normal | |
| colon tumor GW98-560 | 23513 | 35.57 | 9.97 | 19.94 | colon tumor | −1.43 |
| colon normal GW98-894 | 24691 | 33.65 | 24.27 | 48.54 | colon normal | |
| colon tumor GW98-893 | 24690 | 34.34 | 17.53 | 35.06 | colon tumor | −1.38 |
| lung normal GW98-3 | 20742 | 33.59 | 24.98 | 49.95 | lung normal | |
| lung tumor GW98-2 | 20741 | 35.31 | 11.21 | 22.43 | lung tumor | −2.23 |
| lung normal GW97-179 | 20677 | 32.3 | 46.80 | 93.60 | lung normal | |
| lung tumor GW97-178 | 20676 | 33.4 | 27.36 | 54.71 | lung tumor | −1.71 |
| lung normal GW98-165 | 21922 | 33.45 | 26.71 | 53.41 | lung normal | |
| lung tumor GW98-164 | 21921 | 35.08 | 12.45 | 24.91 | lung tumor | −2.14 |
| lung normal GW98-282 | 22584 | 35.51 | 10.24 | 20.49 | lung normal | |
| lung tumor GW98-281 | 22583 | 34.51 | 16.19 | 32.39 | lung tumor | 1.58 |
| breast normal GW00-392 | 28750 | 32.3 | 46.80 | 46.80 | breast normal | |
| breast tumor GW00-391 | 28746 | 31.59 | 66.84 | 133.68 | breast tumor | 2.86 |
| breast normal GW00-413 | 28798 | 31.6 | 66.50 | 66.50 | breast normal | |
| breast tumor GW00-412 | 28797 | 30.56 | 113.74 | 227.47 | breast tumor | 3.42 |
| breast normal GW00- | 27592-95 | 33.29 | 28.84 | 28.8 | breast normal | |
| 235:238 | | | | | | |
| breast tumor GW00- | 27588-91 | 34.2 | 18.72 | 18.72 | breast tumor | −1.54 |
| 231:234 | | | | | | |
| breast normal GW98-621 | 23656 | 31.35 | 75.54 | 151.07 | breast normal | |
| breast tumor GW98-620 | 23655 | 32.59 | 40.55 | 81.10 | breast tumor | −1.86 |
| brain normal BB99-542 | 25507 | 28.84 | 287.95 | 575.91 | brain normal | |
| brain normal BB99-406 | 25509 | 29.79 | 171.25 | 342.49 | brain normal | |
| brain normal BB99-904 | 25546 | 29.31 | 222.20 | 444.40 | brain normal | |
| brain stage 5 ALZ BB99- | 25502 | 30.49 | 118.00 | 235.99 | brain stage 5 ALZ | −1.92 |
| 874 | | | | | | |
| brain stage 5 ALZ BB99- | 25503 | 29.61 | 188.72 | 377.45 | brain stage 5 ALZ | −1.20 |
| 887 | | | | | | |
| brain stage 5 ALZ BB99- | 25504 | 30.64 | 109.07 | 218.13 | brain stage 5 ALZ | −2.08 |
| 862 | | | | | | |
| brain stage 5 ALZ BB99- | 25542 | 29.24 | 230.89 | 461.77 | brain stage 5 ALZ | 1.02 |
| 927 | | | | | | |
| CT lung | normal | 35.75 | 9.19 | 18.39 | CT lung Nml | |
| lung 26 | normal | 32 | 54.36 | | lung 26 Nml | |
| lung 27 | normal | 38.39 | 2.93 | 2.93 | lung 27 Nml | |
| lung 24 | COPD | 36.31 | 7.17 | 7.17 | lung 24 COPD | −1.20 |
| lung 28 | COPD | 37.17 | 4.92 | 4.92 | lung 28 COPD | −1.74 |
| lung 23 | COPD | 40 | 1.52 | 1.52 | lung 23 COPD | −5.66 |
| lung 25 | normal | 37.41 | 4.44 | 4.44 | lung 25 Nml | |
| asthmatic lung ODO3112 | 29321 | 35.92 | 8.52 | 8.52 | asthmatic lung | −1.01 |
| asthmatic lung ODO3433 | 29323 | 35.23 | 11.63 | 23.26 | asthmatic lung | 2.71 |
| asthmatic lung ODO3397 | 29322 | 36.73 | 5.96 | 11.92 | asthmatic lung | 1.39 |
| asthmatic lung ODO4928 | 29325 | 35.86 | 8.75 | 17.50 | asthmatic lung | 2.04 |
| endo cells | control | 36.71 | 6.01 | 6.01 | endo cells | |
| endo VEGF | | 40 | 1.52 | 1.52 | endo VEGF | −3.96 |
| endo bFGF | | 36.68 | 6.09 | 6.09 | endo bFGF | 1.01 |
| heart Clontech | normal | 36.29 | 7.23 | 14.46 | heart | |
| heart (T-1) ischemic | 29417 | 35.59 | 9.88 | 19.76 | heart (T-1) | 1.37 |
| | | | | | ischemic | |
| heart (T-14) non- | 29422 | 34.39 | 17.12 | 34.25 | heart (T-14) non- | 2.37 |
| obstructive DCM | | | | | obstructive DCM | |
| heart (T-3399) DCM | 29426 | 33.35 | 28.02 | 56.04 | heart (T-3399) | 3.88 |
| | | | | | DCM | |
| adenoid GW99-269 | 26162 | 33.95 | 21.05 | 42.10 | adenoid | |
| tonsil GW98-280 | 22582 | 32.25 | 47.98 | 95.96 | tonsil | |
| T cells PC00314 | 28453 | 32.71 | 38.23 | 76.46 | T cells | |
| PBMNC | | 34.51 | 16.19 | 16.19 | PBMNC | |
| monocyte | | 33.47 | 26.45 | 52.90 | monocyte | |
| B cells PC00665 | 28455 | 31.76 | 61.33 | 122.66 | B cells | |
| dendritic cells | 28441 | 40 | 1.52 | 3.04 | dendritic cells | |
| neutrophils | 28440 | 34.62 | 15.39 | 15.39 | neutrophils | |
| eosinophils | 28446 | 35.03 | 12.74 | 25.48 | eosinophils | |
| BM unstim | | 40 | 1.52 | 1.52 | BM unstim | |
| BM stim | treated | 35.21 | 11.74 | 11.74 | BM stim | 7.73 |
| osteo dif | treated | 40 | 1.52 | 1.52 | osteo dif | 1.00 |
| osteo undif | | 40 | 1.52 | 1.52 | osteo undif | |
| chondrocytes | | 33.32 | 28.43 | 71.07 | chondrocytes | |
| OA Synovium IP12/01 | 29462 | 32.37 | 45.20 | 45.20 | OA Synovium | |
| OA Synovium NP10/01 | 29461 | 33.53 | 25.70 | 51.41 | OA Synovium | |
| OA Synovium NP57/00 | 28464 | 32.35 | 45.66 | 91.31 | OA Synovium | |
| RA Synovium | 28466 | 35.01 | 12.86 | 25.72 | RA Synovium | |
| NP03/01 | | | | | | |
| RA Synovium | 28467 | 35 | 12.92 | 25.83 | RA Synovium | |
| NP71/00 | | | | | | |
| RA Synovium | 28475 | 35.89 | 8.64 | 17.27 | RA Synovium | |
| NP45/00 | | | | | | |
| OA bone (biobank) | 29217 | 36.92 | 5.49 | 5.49 | OA bone | |
| | | | | | (biobank) | |
| OA bone Sample 1 | J. Emory | 34.64 | 15.25 | 30.49 | OA bone | |
| OA bone Sample 2 | J. Emory | 34.46 | 16.58 | 33.15 | OA bone | |
| Cartilage (pool) | Normal | 33.85 | 22.07 | 44.14 | Nml Cartilage | |
| | | | | | (pool) | |
| Cartilage (pool) | OA | 37.6 | 4.09 | 8.19 | OA Cartilage | −5.39 |
| | | | | | (pool) | |
| PBL unifected | 28441 | 40 | 1.52 | 3.04 | PBL unifected | |
| PBL HIV IIIB | 28442 | 36.23 | 7.42 | 14.85 | PBL HIV IIIB | 4.89 |
| MRC5 uninfected | 29158 | 40 | 1.52 | 3.04 | MRC5 uninfected | |
| (100%) | | | | | (100%) | |
| MRC5 HSV strain F | 29178 | 40 | 1.52 | 3.04 | MRC5 HSV | 1.00 |
| | | | | | strain F | |
| W12 cells | 29179 | 34.5 | 16.27 | 32.54 | W12 cells | 2.68 |
| Keratinocytes | 29180 | 36.69 | 6.06 | 12.13 | Keratinocytes | |
| B-actin control | | 27 | 828.79 | | | |
| genomic | | 25.75 | 1772.38 | Copy number was calculated from the | | |
| | | | | standard curve run on the normal plate | | |
| 1.00E+05 | | | | | | |
| 1.00E+05 | | | | | | |
| 1.00E+04 | | | | | | |
| 1.00E+04 | | | | | | |
| 1.00E+03 | | | | | | |
| 1.00E+03 | | | | | | |
| 1.00E+02 | | | | | | |
| 1.00E+02 | | | | | | |
| 1.00E+01 | | | | | | |
| 1.00E+01 | | | | | | |
| 1.00E+00 | | | | | | |
| 1.00E+00 | | | | | | |
| NTC | | 40 | 1.52 | | | |

*lung 26 Normal has been omitted due to multiple amplification failures from that sample

Gene Name sbg1530894aCE

| | Fold Change in Disease |
|---|---|
| | Population Relative to |
| Disease tissues | Normal |
| colon tumor | −3.96 |
| colon tumor | −1.31 |
| colon tumor | −1.43 |
| colon tumor | −1.38 |
| lung tumor | −2.23 |
| lung tumor | −1.71 |
| lung tumor | −2.14 |
| lung tumor | 1.58 |
| breast tumor | 2.86 |
| breast tumor | 3.42 |
| breast tumor | −1.54 |
| breast tumor | −1.86 |
| brain stage 5 ALZ | −1.92 |
| brain stage 5 ALZ | −1.20 |
| brain stage 5 ALZ | −2.08 |
| brain stage 5 ALZ | 1.02 |
| lung 24 COPD | −1.20 |
| lung 28 COPD | −1.74 |
| lung 23 COPD | −5.66 |
| asthmatic lung | −1.01 |
| asthmatic lung | 2.71 |
| asthmatic lung | 1.39 |
| asthmatic lung | 2.04 |
| endo VEGF | −3.96 |
| endo bFGF | 1.01 |
| heart (T-1) ischemic | 1.37 |
| heart (T-14) non-obstructive DCM | 2.37 |
| heart (T-3399) DCM | 3.88 |
| BM stim | 7.73 |
| osteo dif | 1.00 |
| OA Cartilage (pool) | −5.39 |
| PBL HIV IIIB | 4.89 |
| MRC5 HSV strain F | 1.00 |
| W12 cells | 2.68 |

Gene Name sbg1530894bCE

Unlike sbg1530894aCE, Sybrman experiment failed using the primers specific for sbg1530894bCE. Taqman was performed by using the primers present in both sbg1530894aCE and sbg1530894bCE and the results is shown here as sbg1530894CE.

Moderate to low overall expression. The highest normal expression is seen in the whole brain, hypothalamus, omentum, ovary, testis and uterus. The highest disease expression is seen in the Alzheimer's disease brain samples. Upregulation in 2 of 4 lung tumor samples and 2 of 4 breast tumor samples implicates this gene in cancers of the lung and breast. Downregulation in the VEGF and bFGF-treated endothelial cells suggests that this gene may be involved in angiogenesis. Upregulation in 2 of 3 disease heart samples implies an involvement in cardiovascular diseases such as ischemic and non-obstructive DCM. Downregulated in the stimulated bone marrow and the differentiated osteoblasts. Downregulation in the HIV-infected PBLs suggests that this gene may be a host factor in HIV.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample | (sample | (sample | GOI | rRNA | 18 S | total |
| sbg1530894CE | 1 and 2) | 1) | 2) | Copies | (ng) | rRNA(ng) | RNA |
| Subcutaneous | 40, 37.59 | 0 | 4.45 | 2.23 | 3.06 | 16.34 | 36.36 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland | 40, 40 | 0 | 0 | 0.00 | 0.61 | 81.97 | 0.00 |
| Clontech | | | | | | | |
| Whole Brain Clontech | 30.6, 30.61 | 341.62 | 340.48 | 341.05 | 7.24 | 6.91 | 2355.32 |
| Fetal Brain Clontech | 37.44, 37.27 | 4.89 | 5.41 | 5.15 | 0.48 | 103.95 | 535.34 |
| Cerebellum Clontech | 40, 37.19 | 0 | 5.71 | 2.86 | 2.17 | 23.04 | 65.78 |
| Cervix | 35.49, 34.95 | 16.35 | 22.86 | 19.61 | 2.42 | 20.66 | 405.06 |
| Colon | 38.35, 40 | 2.77 | 0 | 1.39 | 2.71 | 18.45 | 25.55 |
| Endometrium | 36.78, 36.6 | 7.36 | 8.22 | 7.79 | 0.73 | 68.21 | 531.38 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 35.17, 36.99 | 20.02 | 6.46 | 13.24 | 0.32 | 155.28 | 2055.90 |
| Ileum | 35.18, 36.3 | 19.85 | 9.89 | 14.87 | 2.58 | 19.38 | 288.18 |
| Jejunum | 34.78, 34.01 | 25.48 | 41.1 | 33.29 | 6.60 | 7.58 | 252.20 |
| Kidney | 40, 40 | 0 | 0 | 0.00 | 2.12 | 23.58 | 0.00 |
| Liver | 35.78, 36.65 | 13.69 | 7.98 | 10.84 | 1.50 | 33.33 | 361.17 |
| Fetal Liver Clontech | 40, 40 | 0 | 0 | 0.00 | 10.40 | 4.81 | 0.00 |
| Lung | 40, 40 | 0 | 0 | 0.00 | 2.57 | 19.46 | 0.00 |
| Mammary Gland | 40, 40 | 0 | 0 | 0.00 | 13.00 | 3.85 | 0.00 |
| Clontech | | | | | | | |
| Myometrium | 40, 36.48 | 0 | 8.88 | 4.44 | 2.34 | 21.37 | 94.87 |
| Omentum | 32.21, 31.71 | 126.06 | 171.55 | 148.81 | 3.94 | 12.69 | 1888.39 |
| Ovary | 32.77, 32.21 | 88.67 | 125.55 | 107.11 | 4.34 | 11.52 | 1233.99 |
| Pancreas | 40, 40 | 0 | 0 | 0.00 | 0.81 | 61.80 | 0.00 |
| Head of Pancreas | 40, 40 | 0 | 0.79 | 0.40 | 1.57 | 31.85 | 12.58 |
| Parotid Gland | 35.78, 36.51 | 13.67 | 8.7 | 11.19 | 5.48 | 9.12 | 102.05 |
| Placenta Clontech | 40, 40 | 0 | 0 | 0.00 | 5.26 | 9.51 | 0.00 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland | 40, 37.1 | 0 | 6.04 | 3.02 | 7.31 | 6.84 | 20.66 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 40 | 0 | 0 | 0.00 | 0.98 | 51.07 | 0.00 |
| Clontech | | | | | | | |
| Spleen | 36.29, 35.53 | 10.01 | 15.96 | 12.99 | 4.92 | 10.16 | 131.96 |
| Stomach | 36.68, 38.5 | 7.81 | 2.53 | 5.17 | 2.73 | 18.32 | 94.69 |
| Testis Clontech | 35.69, 35.74 | 14.48 | 14.08 | 14.28 | 0.57 | 87.87 | 1254.83 |
| Thymus Clontech | 35.24, 34.63 | 19.1 | 27.99 | 23.55 | 9.89 | 5.06 | 119.03 |
| Thyroid | 40, 40 | 0 | 0 | 0.00 | 2.77 | 18.05 | 0.00 |
| Trachea Clontech | 40, 40 | 0.35 | 0 | 0.18 | 9.71 | 5.15 | 0.90 |
| Urinary Bladder | 40, 40 | 0 | 0 | 0.00 | 5.47 | 9.14 | 0.00 |
| Uterus | 32.08, 32.36 | 136.79 | 114.5 | 125.65 | 5.34 | 9.36 | 1176.45 |
| genomic | 28.62 | 1173.55 | | | | | |
| b-actin | 29.41 | 718.23 | | | | | |
| 1.00E+05 | 22.09 | 100000 | | | | | |
| 1.00E+05 | 21.39 | 100000 | | | | | |
| 1.00E+04 | 25 | 10000 | | | | | |
| 1.00E+04 | 25.06 | 10000 | | | | | |
| 1.00E+03 | 28.56 | 1000 | | | | | |
| 1.00E+03 | 28.68 | 1000 | | | | | |
| 1.00E+02 | 32.52 | 100 | | | | | |
| 1.00E+02 | 32.29 | 100 | | | | | |
| 1.00E+01 | 37.18 | 10 | | | | | |
| 1.00E+01 | 35.98 | 10 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1530894CE | identifier) | Ct | copies | RNA | Sample | Population |
| colon normal GW98-167 | 21941 | 36.94 | 10.38 | 20.76 | colon normal | |
| colon tumor GW98-166 | 21940 | 40 | 0 | 0.00 | colon tumor | −20.76 |
| colon normal GW98-178 | 22080 | 40 | 0 | 0.00 | colon normal | |
| colon tumor GW98-177 | 22060 | 40 | 0 | 0.00 | colon tumor | 0.00 |
| colon normal GW98-561 | 23514 | 40 | 0 | 0.00 | colon normal | |
| colon tumor GW98-560 | 23513 | 40 | 0 | 0.00 | colon tumor | 0.00 |
| colon normal GW98-894 | 24691 | 40 | 0 | 0.00 | colon normal | |
| colon tumor GW98-893 | 24690 | 40 | 0.97 | 1.94 | colon tumor | 1.94 |
| lung normal GW98-3 | 20742 | 36.92 | 10.53 | 21.06 | lung normal | |
| lung tumor GW98-2 | 20741 | 36.73 | 11.69 | 23.38 | lung tumor | 1.11 |
| lung normal GW97-179 | 20677 | 40 | 0 | 0.00 | lung normal | |
| lung tumor GW97-178 | 20676 | 36.56 | 12.95 | 25.90 | lung tumor | 25.90 |
| lung normal GW98-165 | 21922 | 40 | 0 | 0.00 | lung normal | |
| lung tumor GW98-164 | 21921 | 36.61 | 12.58 | 25.16 | lung tumor | 25.16 |
| lung normal GW98-282 | 22584 | 40 | 0 | 0.00 | lung normal | |
| lung tumor GW98-281 | 22583 | 40 | 0 | 0.00 | lung tumor | 0.00 |
| breast normal GW00-392 | 28750 | 40 | 0 | 0.00 | breast normal | |
| breast tumor GW00-391 | 28746 | 40 | 0 | 0.00 | breast tumor | 0.00 |
| breast normal GW00-413 | 28798 | 34.58 | 40.93 | 40.93 | breast normal | |
| breast tumor GW00-412 | 28797 | 30.68 | 395.58 | 791.16 | breast tumor | 19.33 |
| breast normal GW00- | 27592-95 | 40 | 0 | 0.00 | breast normal | |
| 235:238 | | | | | | |
| breast tumor GW00- | 27588-91 | 36.11 | 16.78 | 16.78 | breast tumor | 16.78 |
| 231:234 | | | | | | |
| breast normal GW98-621 | 23656 | 40 | 0 | 0.00 | breast normal | |
| breast tumor GW98-620 | 23655 | 40 | 0 | 0.00 | breast tumor | 0.00 |
| brain normal BB99-542 | 25507 | 40 | 1.4 | 2.80 | brain normal | |
| brain normal BB99-406 | 25509 | 31.15 | 300.7 | 601.40 | brain normal | |
| brain normal BB99-904 | 25546 | 30.99 | 330.39 | 660.78 | brain normal | |
| brain stage 5 ALZ BB99- | 25502 | 32.9 | 108.75 | 217.50 | brain stage 5 ALZ | −1.94 |
| 874 | | | | | | |
| brain stage 5 ALZ BB99- | 25503 | 32.2 | 163.47 | 326.94 | brain stage 5 ALZ | −1.29 |
| 887 | | | | | | |
| brain stage 5 ALZ BB99- | 25504 | 32.28 | 156.13 | 312.26 | brain stage 5 ALZ | −1.35 |
| 862 | | | | | | |
| brain stage 5 ALZ BB99- | 25542 | 31.19 | 292.69 | 585.38 | brain stage 5 ALZ | 1.39 |
| 927 | | | | | | |
| CT lung | normal | 40 | 0 | 0.00 | CT lung Nml | |
| lung 26 | normal | 40 | 0 | | lung 26 Nml | |
| lung 27 | normal | 40 | 0 | 0.00 | lung 27 Nml | |
| lung 24 | COPD | 36.44 | 13.88 | 13.88 | lung 24 COPD | 2.16 |
| lung 28 | COPD | 40 | 0 | 0.00 | lung 28 COPD | −6.41 |
| lung 23 | COPD | 40 | 0 | 0.00 | lung 23 COPD | −6.41 |
| lung 25 | normal | 35.88 | 19.24 | 19.24 | lung 25 Nml | |
| asthmatic lung ODO3112 | 29321 | 37.87 | 6.05 | 6.05 | asthmatic lung | −1.06 |
| asthmatic lung ODO3433 | 29323 | 40 | 0 | 0.00 | asthmatic lung | −6.41 |
| asthmatic lung ODO3397 | 29322 | 37.04 | 9.8 | 19.60 | asthmatic lung | 3.06 |
| asthmatic lung ODO4928 | 29325 | 36.4 | 14.17 | 28.34 | asthmatic lung | 4.42 |
| endo cells | control | 37.07 | 9.63 | 9.63 | endo cells | |
| endo VEGF | | 40 | 0 | 0.00 | endo VEGF | −9.63 |
| endo bFGF | | 40 | 0 | 0.00 | endo bFGF | −9.63 |
| heart Clontech | normal | 40 | 0 | 0.00 | heart | |
| heart (T-1) ischemic | 29417 | 36.25 | 15.47 | 30.94 | heart (T-1) | 30.94 |
| | | | | | ischemic | |
| heart (T-14) non- | 29422 | 37.11 | 9.4 | 18.80 | heart (T-14) non- | 18.80 |
| obstructive DCM | | | | | obstructive DCM | |
| heart (T-3399) DCM | 29426 | 40 | 0 | 0.00 | heart (T-3399) | 0.00 |
| | | | | | DCM | |
| adenoid GW99-269 | 26162 | 34.58 | 40.89 | 81.78 | adenoid | |
| tonsil GW98-280 | 22582 | 40 | 0 | 0.00 | tonsil | |
| T cells PC00314 | 28453 | 40 | 0 | 0.00 | T cells | |
| PBMNC | | 40 | 0 | 0.00 | PBMNC | |
| monocyte | | 35.66 | 21.89 | 43.78 | monocyte | |
| B cells PC00665 | 28455 | 35.23 | 28.01 | 56.02 | B cells | |
| dendritic cells | 28441 | 35.07 | 30.82 | 61.64 | dendritic cells | |
| neutrophils | 28440 | 40 | 0 | 0.00 | neutrophils | |
| eosinophils | 28446 | 40 | 0 | 0.00 | eosinophils | |
| BM unstim | | 38.2 | 5 | 5.00 | BM unstim | |
| BM stim | treated | 40 | 0 | 0.00 | BM stim | −5.00 |
| osteo dif | treated | 40 | 0 | 0.00 | osteo dif | −11.25 |
| osteo undif | | 36.8 | 11.25 | 11.25 | osteo undif | |
| chondrocytes | | 35.73 | 20.99 | 52.48 | chondrocytes | |
| OA Synovium IP12/01 | 29462 | 35.04 | 31.38 | 31.38 | OA Synovium | |
| OA Synovium NP10/01 | 29461 | 35.86 | 19.44 | 38.88 | OA Synovium | |
| OA Synovium NP57/00 | 28464 | 38.53 | 4.12 | 8.24 | OA Synovium | |
| RA Synovium | 28466 | 40 | 0 | 0.00 | RA Synovium | |
| NP03/01 | | | | | | |
| RA Synovium | 28467 | 40 | 0 | 0.00 | RA Synovium | |
| NP71/00 | | | | | | |
| RA Synovium | 28475 | 40 | 0 | 0.00 | RA Synovium | |
| NP45/00 | | | | | | |
| OA bone (biobank) | 29217 | 35.47 | 24.34 | 24.34 | OA bone (biobank) | |
| OA bone Sample 1 | J. Emory | 40 | 0 | 0.00 | OA bone | |
| OA bone Sample 2 | J. Emory | 36.49 | 13.49 | 26.98 | OA bone | |
| Cartilage (pool) | Normal | 40 | 0 | 0.00 | Nml Cartilage | |
| | | | | | (pool) | |
| Cartilage (pool) | OA | 40 | 0 | 0.00 | OA Cartilage (pool) | 0.00 |
| PBL unifected | 28441 | 35.8 | 20.15 | 40.30 | PBL unifected | |
| PBL HIV IIIB | 28442 | 40 | 0 | 0.00 | PBL HIV IIIB | −40.30 |
| MRC5 uninfected | 29158 | 40 | 0 | 0.00 | MRC5 uninfected | |
| (100%) | | | | | (100%) | |
| MRC5 HSV strain F | 29178 | 40 | 0 | 0.00 | MRC5 HSV strain | 0.00 |
| | | | | | F | |
| W12 cells | 29179 | 36.77 | 11.43 | 22.86 | W12 cells | 1.10 |
| Keratinocytes | 29180 | 36.94 | 10.36 | 20.72 | Keratinocytes | |
| B-actin control | | 29.12 | 977.73 | | | |
| genomic | | 28.27 | 1599.71 | | | |
| 1.00E+05 | | 21.56 | 100000 | | | |
| 1.00E+05 | | 21.26 | 100000 | | | |
| 1.00E+04 | | 24.94 | 10000 | | | |
| 1.00E+04 | | 25.13 | 10000 | | | |
| 1.00E+03 | | 28.75 | 1000 | | | |
| 1.00E+03 | | 28.57 | 1000 | | | |
| 1.00E+02 | | 33.51 | 100 | | | |
| 1.00E+02 | | 32.7 | 100 | | | |
| 1.00E+01 | | 40 | 0 | | | |
| 1.00E+01 | | 37.37 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1530894CE

| | Fold Change in Disease |
|---|---|
| | Population Relative to |
| Disease tissues | Normal |
| colon tumor | −20.76 |
| colon tumor | 0.00 |
| colon tumor | 0.00 |
| colon tumor | 1.94 |
| lung tumor | 1.11 |
| lung tumor | 25.90 |
| lung tumor | 25.16 |
| lung tumor | 0.00 |
| breast tumor | 0.00 |
| breast tumor | 19.33 |
| breast tumor | 16.78 |
| breast tumor | 0.00 |
| brain stage 5 ALZ | −1.94 |
| brain stage 5 ALZ | −1.29 |
| brain stage 5 ALZ | −1.35 |
| brain stage 5 ALZ | 1.39 |
| lung 24 COPD | 2.16 |
| lung 28 COPD | −6.41 |
| lung 23 COPD | −6.41 |
| asthmatic lung | −1.06 |
| asthmatic lung | −6.41 |
| asthmatic lung | 3.06 |
| asthmatic lung | 4.42 |
| endo VEGF | −9.63 |
| endo bFGF | −9.63 |
| heart (T-1) ischemic | 30.94 |
| heart (T-14) non-obstructive DCM | 18.80 |
| heart (T-3399) DCM | 0.00 |
| BM stim | −5.00 |
| osteo dif | −11.25 |
| OA Cartilage (pool) | 0.00 |
| PBL HIV IIIB | −40.30 |
| MRC5 HSV strain F | 0.00 |
| W12 cells | 1.10 |

Gene Name gsk.1813134.NaI

Moderate to low overall expression. The highest normal expression is seen in the thyroid and trachea. The highest disease expression is seen in two of the lung tumor samples. Upregulation in 2 of 4 lung tumor samples implicates this gene in cancer of lung. Downregulation in 1 of 4 Alzheimer's brain samples suggests an involvement in Alzheimer's disease. Upregulation in 1 of 3 COPD lung samples and in 2 of 4 asthmatic lung samples implicates this gene in COPD and asthma.

| | | | | | | | copies of |
|---|---|---|---|---|---|---|---|
| | | Mean | Mean | | | | mRNA |
| | | GOI | GOI | | | | detected/ |
| | Ct | copies | copies | Average | 18 S | 50 ng/ | 50 ng |
| Sample | (sample | (sample | (sample | GOI | rRNA | 18 S | total |
| gsk.1813134.NaI | 1 and 2) | 1) | 2) | Copies | (ng) | rRNA(ng) | RNA |
| Subcutaneous | 39:29, 40 | 8 | 0 | 4.00 | 3.06 | 16.34 | 65.36 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 39.7 | 2.37 | 6.35 | 4.36 | 0.61 | 81.97 | 357.38 |
| Whole Brain Clontech | 34.03, 34.33 | 154.47 | 130.6 | 142.54 | 7.24 | 6.91 | 984.36 |
| Fetal Brain Clontech | 37.91, 37.87 | 17.35 | 17.75 | 17.55 | 0.48 | 103.95 | 1824.32 |
| Cerebellum Clontech | 39.61, 40 | 6.66 | 3.17 | 4.92 | 2.17 | 23.04 | 113.25 |
| Cervix | 36.4, 37.11 | 40.8 | 27.25 | 34.03 | 2.42 | 20.66 | 703.00 |
| Colon | 39.3, 39.91 | 7.96 | 3.98 | 5.97 | 2.71 | 18.45 | 110.15 |
| Endometrium | 39.7, 40 | 6.34 | 1.89 | 4.12 | 0.73 | 68.21 | 280.70 |
| Esophagus | 38.1, 40 | 15.6 | 0 | 7.80 | 1.37 | 36.50 | 284.67 |
| Heart Clontech | 39.17, 40 | 8.55 | 0 | 4.28 | 1.32 | 37.88 | 161.93 |
| Hypothalamus | 40, 40 | 3.05 | 0 | 1.53 | 0.32 | 155.28 | 236.80 |
| Ileum | 40, 39.82 | 1.82 | 5.93 | 3.88 | 2.58 | 19.38 | 75.10 |
| Jejunum | 40, 37.92 | 1.83 | 17.31 | 9.57 | 6.60 | 7.58 | 72.50 |
| Kidney | 35.47, 35 | 68.81 | 89.81 | 79.31 | 2.12 | 23.58 | 1870.52 |
| Liver | 40, 40 | 0 | 2.76 | 1.38 | 1.50 | 33.33 | 46.00 |
| Fetal Liver Clontech | 40, 40 | 2.33 | 0 | 1.17 | 10.40 | 4.81 | 5.60 |
| Lung | 39.51, 38.29 | 7.05 | 14.04 | 10.55 | 2.57 | 19.46 | 205.16 |
| Mammary Gland | 39.43, 37.45 | 7.36 | 22.58 | 14.97 | 13.00 | 3.85 | 57.58 |
| Clontech | | | | | | | |
| Myometrium | 39.76, 40 | 6.11 | 1.86 | 3.99 | 2.34 | 21.37 | 85.15 |
| Omentum | 39.02, 40 | 9.28 | 2.45 | 5.87 | 3.94 | 12.69 | 74.43 |
| Ovary | 37.12, 37.59 | 27.16 | 20.76 | 23.96 | 4.34 | 11.52 | 276.04 |
| Pancreas | 37.57, 39.96 | 21.06 | 2.66 | 11.86 | 0.81 | 61.80 | 733.00 |
| Head of Pancreas | 39.56, 40 | 6.86 | 1.83 | 4.35 | 1.57 | 31.85 | 138.38 |
| Parotid Gland | 39.41, 38.97 | 7.44 | 9.56 | 8.50 | 5.48 | 9.12 | 77.55 |
| Placenta Clontech | 39.21, 40 | 8.35 | 0 | 4.18 | 5.26 | 9.51 | 39.69 |
| Prostate | 39.02, 40 | 9.27 | 2.29 | 5.78 | 3.00 | 16.67 | 96.33 |
| Rectum | 39.5, 40 | 7.1 | 2.18 | 4.64 | 1.23 | 40.65 | 188.62 |
| Salivary Gland | 34.61, 33.52 | 111.69 | 206.39 | 159.04 | 7.31 | 6.84 | 1087.82 |
| Clontech | | | | | | | |
| Skeletal Muscle | 40, 40 | 2.13 | 0 | 1.07 | 1.26 | 39.68 | 42.26 |
| Clontech | | | | | | | |
| Skin | 40, 40 | 0 | 0 | 0.00 | 1.21 | 41.32 | 0.00 |
| Small Intestine | 37.24, 40 | 25.41 | 0 | 12.71 | 0.98 | 51.07 | 648.88 |
| Clontech | | | | | | | |
| Spleen | 39.84, 40 | 5.85 | 2.3 | 4.08 | 4.92 | 10.16 | 41.41 |
| Stomach | 39.71, 39.83 | 6.32 | 5.9 | 6.11 | 2.73 | 18.32 | 111.90 |
| Testis Clontech | 40, 39.2 | 0 | 8.38 | 4.19 | 0.57 | 87.87 | 368.19 |
| Thymus Clontech | 35.22, 34.51 | 78.98 | 117.98 | 98.48 | 9.89 | 5.06 | 497.88 |
| Thyroid | 31.49, 31.43 | 648.86 | 672.22 | 660.54 | 2.77 | 18.05 | 11923.10 |
| Trachea Clontech | 30.33, 29.88 | 1245.44 | 1605.59 | 1425.52 | 9.71 | 5.15 | 7340.45 |
| Urinary Bladder | 38.79, 39.24 | 10.56 | 8.22 | 9.39 | 5.47 | 9.14 | 85.83 |
| Uterus | 38.49, 37.08 | 12.55 | 27.76 | 20.16 | 5.34 | 9.36 | 188.72 |
| genomic | 32.01 | 484.64 | | | | | |
| b-actin | 27.44 | 6351.66 | | | | | |
| 1.00E+05 | 21.36 | 100000 | | | | | |
| 1.00E+05 | 21.76 | 100000 | | | | | |
| 1.00E+04 | 25.98 | 10000 | | | | | |
| 1.00E+04 | 26.16 | 10000 | | | | | |
| 1.00E+03 | 31.16 | 1000 | | | | | |
| 1.00E+03 | 31.66 | 1000 | | | | | |
| 1.00E+02 | 37.46 | 100 | | | | | |
| 1.00E+02 | 37.32 | 100 | | | | | |
| 1.00E+01 | 40 | 0 | | | | | |
| 1.00E+01 | 40 | 0 | | | | | |
| 1.00E+00 | 39.55 | 1 | | | | | |
| 1.00E+00 | 40 | 1 | | | | | |
| NTC | 40 | 0 | | | | | |
| NTC | 40 | −1 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| gsk.1813134.NaI | identifier) | Ct | copies | RNA | Sample | Population |
| colon normal GW98-167 | 21941 | 38.23 | 9.78 | 19.56 | colon normal | |
| colon tumor GW98-166 | 21940 | 39.42 | 5.31 | 10.62 | colon tumor | −1.84 |
| colon normal GW98-178 | 22080 | 40 | 3.97 | 7.94 | colon normal | |
| colon tumor GW98-177 | 22060 | 38.84 | 7.14 | 14.27 | colon tumor | 1.80 |
| colon normal GW98-561 | 23514 | 33.81 | 113.02 | 226.03 | colon normal | |
| colon tumor GW98-560 | 23513 | 37.18 | 17.03 | 34.07 | colon tumor | −6.63 |
| colon normal GW98-894 | 24691 | 35.9 | 34.22 | 68.45 | colon normal | |
| colon tumor GW98-893 | 24690 | 37.53 | 14.13 | 28.27 | colon tumor | −2.42 |
| lung normal GW98-3 | 20742 | 32.09 | 319.70 | 639.41 | lung normal | |
| lung tumor GW98-2 | 20741 | 38.85 | 7.10 | 14.20 | lung tumor | −45.03 |
| lung normal GW97-179 | 20677 | 32.76 | 211.84 | 423.69 | lung normal | |
| lung tumor GW97-178 | 20676 | 28.14 | 4374.08 | 8748.15 | lung tumor | 20.65 |
| lung normal GW98-165 | 21922 | 31.8 | 383.06 | 766.13 | lung normal | |
| lung tumor GW98-164 | 21921 | 29.02 | 2368.86 | 4737.72 | lung tumor | 6.18 |
| lung normal GW98-282 | 22584 | 33.45 | 139.87 | 279.74 | lung normal | |
| lung tumor GW98-281 | 22583 | 32.45 | 256.01 | 512.02 | lung tumor | 1.83 |
| breast normal GW00-392 | 28750 | 35.3 | 47.88 | 47.88 | breast normal | |
| breast tumor GW00-391 | 28746 | 37.96 | 11.26 | 22.53 | breast tumor | −2.13 |
| breast normal GW00-413 | 28798 | 37.35 | 15.55 | 15.55 | breast normal | |
| breast tumor GW00-412 | 28797 | 36.67 | 22.43 | 44.85 | breast tumor | 2.88 |
| breast normal GW00- | 27592-95 | 39.85 | 4.28 | 4.28 | breast normal | |
| 235:238 | | | | | | |
| breast tumor GW00- | 27588-91 | 37.01 | 18.66 | 18.66 | breast tumor | 4.36 |
| 231:234 | | | | | | |
| breast normal GW98-621 | 23656 | 37.84 | 12.00 | 24.00 | breast normal | |
| breast tumor GW98-620 | 23655 | 36.43 | 25.56 | 51.12 | breast tumor | 2.13 |
| brain normal BB99-542 | 25507 | 34.54 | 73.85 | 147.71 | brain normal | |
| brain normal BB99-406 | 25509 | 37.19 | 16.94 | 33.89 | brain normal | |
| brain normal BB99-904 | 25546 | 36.59 | 23.42 | 46.85 | brain normal | |
| brain stage 5 ALZ BB99- | 25502 | 40 | 3.97 | 7.94 | brain stage 5 | −9.59 |
| 874 | | | | | ALZ | |
| brain stage 5 ALZ BB99- | 25503 | 35.11 | 53.31 | 106.62 | brain stage 5 | 1.40 |
| 887 | | | | | ALZ | |
| brain stage 5 ALZ BB99- | 25504 | 36.17 | 29.48 | 58.96 | brain stage 5 | −1.29 |
| 862 | | | | | ALZ | |
| brain stage 5 ALZ BB99- | 25542 | 36.63 | 22.92 | 45.84 | brain stage 5 | −1.66 |
| 927 | | | | | ALZ | |
| CT lung | normal | 34.87 | 61.11 | 122.22 | CT lung Nml | |
| lung 26 | normal | 31.7 | 407.86 | | lung 26 Nml | |
| lung 27 | normal | 34.62 | 70.53 | 70.53 | lung 27 Nml | |
| lung 24 | COPD | 34.04 | 98.74 | 98.74 | lung 24 COPD | 1.24 |
| lung 28 | COPD | 31.97 | 344.47 | 344.47 | lung 28 COPD | 4.31 |
| lung 23 | COPD | 34.82 | 62.88 | 62.88 | lung 23 COPD | −1.27 |
| lung 25 | normal | 35.34 | 46.81 | 46.81 | lung 25 Nml | |
| asthmatic lung ODO3112 | 29321 | 37.88 | 11.75 | 11.75 | asthmatic lung | −6.80 |
| asthmatic lung ODO3433 | 29323 | 34.95 | 58.39 | 116.77 | asthmatic lung | 1.46 |
| asthmatic lung ODO3397 | 29322 | 33.25 | 157.62 | 315.23 | asthmatic lung | 3.95 |
| asthmatic lung ODO4928 | 29325 | 31.48 | 468.54 | 937.09 | asthmatic lung | 11.74 |
| endo cells | control | 38.75 | 7.47 | 7.47 | endo cells | |
| endo VEGF | | 39.62 | 4.80 | 4.80 | endo VEGF | −1.56 |
| endo bFGF | | 38.7 | 7.67 | 7.67 | endo bFGF | 1.03 |
| heart Clontech | normal | 40 | 3.97 | 7.94 | heart | |
| heart (T-1) ischemic | 29417 | 39.83 | 4.32 | 8.65 | heart (T-1) | 1.09 |
| | | | | | ischemic | |
| heart (T-14) non- | 29422 | 40 | 3.97 | 7.94 | heart (T-14) | 1.00 |
| obstructive DCM | | | | | non-obstructive | |
| | | | | | DCM | |
| heart (T-3399) DCM | 29426 | 40 | 3.97 | 7.94 | heart (T-3399) | 1.00 |
| | | | | | DCM | |
| adenoid GW99-269 | 26162 | 37.3 | 15.97 | 31.95 | adenoid | |
| tonsil GW98-280 | 22582 | 34.42 | 79.15 | 158.31 | tonsil | |
| T cells PC00314 | 28453 | 37.26 | 16.32 | 32.64 | T cells | |
| PBMNC | | 40 | 3.97 | 3.97 | PBMNC | |
| monocyte | | 40 | 3.97 | 7.94 | monocyte | |
| B cells PC00665 | 28455 | 38.12 | 10.36 | 20.72 | B cells | |
| dendritic cells | 28441 | 38.54 | 8.33 | 16.66 | dendritic cells | |
| neutrophils | 28440 | 37.99 | 11.09 | 11.09 | neutrophils | |
| eosinophils | 28446 | 38.59 | 8.12 | 16.23 | eosinophils | |
| BM unstim | | 39.48 | 5.15 | 5.15 | BM unstim | |
| BM stim | treated | 40 | 3.97 | 3.97 | BM stim | −1.30 |
| osteo dif | treated | 40 | 3.97 | 3.97 | osteo dif | 1.00 |
| osteo undif | | 40 | 3.97 | 3.97 | osteo undif | |
| chondrocytes | | 39.71 | 4.59 | 11.48 | chondrocytes | |
| OA Synovium IP12/01 | 29462 | 40 | 3.97 | 3.97 | OA Synovium | |
| OA Synovium NP10/01 | 29461 | 40 | 3.97 | 7.94 | OA Synovium | |
| OA Synovium NP57/00 | 28464 | 39.68 | 4.66 | 9.32 | OA Synovium | |
| RA Synovium | 28466 | 39.23 | 5.85 | 11.70 | RA Synovium | |
| NP03/01 | | | | | | |
| RA Synovium | 28467 | 39.08 | 6.31 | 12.63 | RA Synovium | |
| NP71/00 | | | | | | |
| RA Synovium | 28475 | 38.48 | 8.59 | 17.18 | RA Synovium | |
| NP45/00 | | | | | | |
| OA bone (biobank) | 29217 | 38.7 | 7.67 | 7.67 | OA bone | |
| | | | | | (biobank) | |
| OA bone Sample 1 | J. Emory | 40 | 3.97 | 7.94 | OA bone | |
| OA bone Sample 2 | J. Emory | 40 | 3.97 | 7.94 | OA bone | |
| Cartilage (pool) | Normal | 40 | 3.97 | 7.94 | Nml Cartilage | |
| | | | | | (pool) | |
| Cartilage (pool) | OA | 40 | 3.97 | 7.94 | OA Cartilage | 1.00 |
| | | | | | (pool) | |
| PBL unifected | 28441 | 37.47 | 14.59 | 29.18 | PBL unifected | |
| PBL HIV IIIB | 28442 | 37.62 | 13.48 | 26.95 | PBL HIV IIIB | −1.08 |
| MRC5 uninfected (100%) | 29158 | 38.34 | 9.24 | 18.48 | MRC5 | |
| | | | | | uninfected | |
| | | | | | (100%) | |
| MRC5 HSV strain F | 29178 | 39.31 | 5.62 | 11.23 | MRC5 HSV | −1.64 |
| | | | | | strain F | |
| W12 cells | 29179 | 37.81 | 12.19 | 24.38 | W12 cells | −1.35 |
| Keratinocytes | 29180 | 37.25 | 16.41 | 32.81 | Keratinocytes | |
| B-actin control | | 27.36 | 7656.40 | | | |
| genomic | | 32.01 | 336.00 | Disease plate did not have genomic | | |
| | | | | samples. Copy number was, therefore | | |
| | | | | calculated from the gene- | | |
| | | | | specific genomic curve on the normal | | |
| | | | | plate. | | |
| 1.00E+05 | | | | | | |
| 1.00E+05 | | | | | | |
| 1.00E+04 | | | | | | |
| 1.00E+04 | | | | | | |
| 1.00E+03 | | | | | | |
| 1.00E+03 | | | | | | |
| 1.00E+02 | | | | | | |
| 1.00E+02 | | | | | | |
| 1.00E+01 | | | | | | |
| 1.00E+01 | | | | | | |
| 1.00E+00 | | | | | | |
| 1.00E+00 | | | | | | |
| NTC | | 39.84 | 4.30 | | | |

*lung 26 Normal has been omitted due to multiple amplification failures from that sample

Gene Name gsk.1813134.NaI

| | Fold Change in Disease |
|---|---|
| | Population Relative to |
| Disease tissues | Normal |
| colon tumor | −1.84 |
| colon tumor | 1.80 |
| colon tumor | −6.63 |
| colon tumor | −2.42 |
| lung tumor | −45.03 |
| lung tumor | 20.65 |
| lung tumor | 6.18 |
| lung tumor | 1.83 |
| breast tumor | −2.13 |
| breast tumor | 2.88 |
| breast tumor | 4.36 |
| breast tumor | 2.13 |
| brain stage 5 ALZ | −9.59 |
| brain stage 5 ALZ | 1.40 |
| brain stage 5 ALZ | −1.29 |
| brain stage 5 ALZ | −1.66 |
| lung 24 COPD | 1.24 |
| lung 28 COPD | 4.31 |
| lung 23 COPD | −1.27 |
| asthmatic lung | −6.80 |
| asthmatic lung | 1.46 |
| asthmatic lung | 3.95 |
| asthmatic lung | 11.74 |
| endo VEGF | −1.56 |
| endo bFGF | 1.03 |
| heart (T-1) ischemic | 1.09 |
| heart (T-14) non-obstructive DCM | 1.00 |
| heart (T-3399) DCM | 1.00 |
| BM stim | −1.30 |
| osteo dif | 1.00 |
| OA Cartilage (pool) | 1.00 |
| PBL HIV IIIB | −1.08 |
| MRC5 HSV strain F | −1.64 |
| W12 cells | −1.35 |

Gene Name gsk1811484BrCaAg

Moderate overall expression. The highest normal expression is seen in the testis, prostate, omentum, stomach, thymus, trachea, and uterus. The highest disease expression is seen in the normal and tumor breast samples. Upregulation in 2 of 4 colon tumor samples, 2 of 4 lung tumor samples, and 2 of 4 breast tumor samples implicates this gene in cancers of the colon, lung, and breast. Downregulated in the differentiated osteoblasts. Upregulation in the HSV-infected MRC5 cells and the W12 cells suggests that this gene may be a host factor in HSV and HPV. Patterns of expression are very similar to gsk1810944BrCaAg.

| | | | | | | | copies of |
|---|---|---|---|---|---|---|---|
| | | Mean | Mean | | | | mRNA |
| | | GOI | GOI | | | | detected/ |
| Sample | Ct | copies | copies | Average | 18 S | 50 ng/ | 50 ng |
| gsk1811484- | (sample | (sample | (sample | GOI | rRNA | 18 S | total |
| BrCaAg | 1 and 2) | 1) | 2) | Copies | (ng) | rRNA(ng) | RNA |
| Subcutaneous | 39.41, 36.8 | 0.27 | 1.18 | 0.73 | 3.06 | 16.34 | 11.85 |
| Adipocytes Zenbio | | | | | | | |
| Subcutaneous Adipose | 40, 40 | 0.09 | 0.07 | 0.08 | 0.96 | 52.36 | 4.19 |
| Zenbio | | | | | | | |
| Adrenal Gland Clontech | 36.01, 40 | 1.84 | 0.06 | 0.95 | 0.61 | 81.97 | 77.87 |
| Whole Brain Clontech | 30.18, 30.41 | 49.07 | 43.24 | 46.16 | 7.24 | 6.91 | 318.75 |
| Fetal Brain Clontech | 39.33, 40 | 0.28 | 0.08 | 0.18 | 0.48 | 103.95 | 18.71 |
| Cerebellum Clontech | 37.02, 35.64 | 1.04 | 2.26 | 1.65 | 2.17 | 23.04 | 38.02 |
| Cervix | 33.68, 32.84 | 6.86 | 10.99 | 8.93 | 2.42 | 20.66 | 184.40 |
| Colon | 33.68, 33.16 | 6.84 | 9.18 | 8.01 | 2.71 | 18.45 | 147.79 |
| Endometrium | 33.7, 34.24 | 6.77 | 5 | 5.89 | 0.73 | 68.21 | 401.43 |
| Esophagus | 34.21, 33.46 | 5.06 | 7.76 | 6.41 | 1.37 | 36.50 | 233.94 |
| Heart Clontech | 37.2, 38.49 | 0.94 | 0.46 | 0.70 | 1.32 | 37.88 | 26.52 |
| Hypothalamus | 39.01, 40 | 0.34 | 0 | 0.17 | 0.32 | 155.28 | 26.40 |
| Ileum | 33.93, 34 | 5.96 | 5.7 | 5.83 | 2.58 | 19.38 | 112.98 |
| Jejunum | 30.69, 30.64 | 36.85 | 38.02 | 37.44 | 6.60 | 7.58 | 283.60 |
| Kidney | 40, 38.78 | 0.1 | 0.39 | 0.25 | 2.12 | 23.58 | 5.78 |
| Liver | 32.03, 32.1 | 17.32 | 16.62 | 16.97 | 1.50 | 33.33 | 565.67 |
| Fetal Liver Clontech | 33.75, 35.03 | 6.57 | 3.2 | 4.89 | 10.40 | 4.81 | 23.49 |
| Lung | 36.68, 37.66 | 1.27 | 0.73 | 1.00 | 2.57 | 19.46 | 19.46 |
| Mammary Gland | 31.08, 30.38 | 29.56 | 43.88 | 36.72 | 13.00 | 3.85 | 141.23 |
| Clontech | | | | | | | |
| Myometrium | 34.36, 35.1 | 4.66 | 3.07 | 3.87 | 2.34 | 21.37 | 82.59 |
| Omentum | 29.38, 29.06 | 77.21 | 92.63 | 84.92 | 3.94 | 12.69 | 1077.66 |
| Ovary | 30.52, 30.53 | 40.65 | 40.27 | 40.46 | 4.34 | 11.52 | 466.13 |
| Pancreas | 37.35, 39.03 | 0.87 | 0.34 | 0.61 | 0.81 | 61.80 | 37.39 |
| Head of Pancreas | 37.45, 34.71 | 0.82 | 3.82 | 2.32 | 1.57 | 31.85 | 73.89 |
| Parotid Gland | 30.71, 31.42 | 36.46 | 24.4 | 30.43 | 5.48 | 9.12 | 277.65 |
| Placenta Clontech | 29.42, 29.08 | 75.34 | 91.24 | 83.29 | 5.26 | 9.51 | 791.73 |
| Prostate | 27.78, 27.94 | 189.85 | 173.17 | 181.51 | 3.00 | 16.67 | 3025.17 |
| Rectum | 34.4436.26, | 4.46 | 1.6 | 3.03 | 1.23 | 40.65 | 123.17 |
| Salivary Gland Clontech | 31.26, 31.02 | 26.82 | 30.61 | 28.72 | 7.31 | 6.84 | 196.41 |
| Skeletal Muscle | 37.04, 39.4 | 1.03 | 0.27 | 0.65 | 1.26 | 39.68 | 25.79 |
| Clontech | | | | | | | |
| Skin | 34.33, 36.69 | 4.74 | 1.26 | 3.00 | 1.21 | 41.32 | 123.97 |
| Small Intestine Clontech | 40, 40 | 0 | 0 | 0.00 | 0.98 | 51.07 | 0.00 |
| Spleen | 34.01, 32.49 | 5.69 | 13.41 | 9.55 | 4.92 | 10.16 | 97.05 |
| Stomach | 30.53, 30.18 | 40.32 | 49.15 | 44.74 | 2.73 | 18.32 | 819.32 |
| Testis Clontech | 28.7, 28.22 | 113.42 | 147.88 | 130.65 | 0.57 | 87.87 | 11480.67 |
| Thymus Clontech | 28.18, 28.12 | 151.56 | 157.02 | 154.29 | 9.89 | 5.06 | 780.03 |
| Thyroid | 35.21, 35.05 | 2.89 | 3.16 | 3.03 | 2.77 | 18.05 | 54.60 |
| Trachea Clontech | 28.07, 27.71 | 161.13 | 197.69 | 179.41 | 9.71 | 5.15 | 923.84 |
| Urinary Bladder | 34.85, 33.58 | 3.54 | 7.24 | 5.39 | 5.47 | 9.14 | 49.27 |
| Uterus | 29.88, 29.45 | 58.18 | 74.16 | 66.17 | 5.34 | 9.36 | 619.57 |
| genomic | 23.84 | 1753.28 | | | | | |
| b-actin | 27.13 | 273.51 | | | | | |
| 1.00E+05 | 17.55 | 100000 | | | | | |
| 1.00E+05 | 17.52 | 100000 | | | | | |
| 1.00E+04 | 20.49 | 10000 | | | | | |
| 1.00E+04 | 20.52 | 10000 | | | | | |
| 1.00E+03 | 24.1 | 1000 | | | | | |
| 1.00E+03 | 24.16 | 1000 | | | | | |
| 1.00E+02 | 28.95 | 100 | | | | | |
| 1.00E+02 | 28.17 | 100 | | | | | |
| 1.00E+01 | 31.73 | 10 | | | | | |
| 1.00E+01 | 33.09 | 10 | | | | | |
| 1.00E+00 | 40 | 1 | | | | | |
| 1.00E+00 | 39.14 | 1 | | | | | |
| NTC | 38.79 | −1 | | | | | |
| NTC | 40 | −1 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| gsk1811484- | (GSK | | GOI | total | | Disease |
| BrCaAg | identifier) | Ct | copies | RNA | Sample | Population |
| colon normal GW98-167 | 21941 | 32.98 | 4.15 | 8.30 | colon normal | |
| colon tumor GW98-166 | 21940 | 25.98 | 356.34 | 712.68 | colon tumor | 85.87 |
| colon normal GW98-178 | 22080 | 34.38 | 1.71 | 3.42 | colon normal | |
| colon tumor GW98-177 | 22060 | 32.48 | 5.72 | 11.44 | colon tumor | 3.35 |
| colon normal GW98-561 | 23514 | 34.77 | 1.34 | 2.68 | colon normal | |
| colon tumor GW98-560 | 23513 | 33.92 | 2.28 | 4.56 | colon tumor | 1.70 |
| colon normal GW98-894 | 24691 | 32.14 | 7.11 | 14.22 | colon normal | |
| colon tumor GW98-893 | 24690 | 29.16 | 47.05 | 94.10 | colon tumor | 6.62 |
| lung normal GW98-3 | 20742 | 31.62 | 9.86 | 19.72 | lung normal | |
| lung tumor GW98-2 | 20741 | 24.78 | 762.34 | 1524.68 | lung tumor | 77.32 |
| lung normal GW97-179 | 20677 | 28.13 | 90.81 | 181.62 | lung normal | |
| lung tumor GW97-178 | 20676 | 31.47 | 10.85 | 21.70 | lung tumor | −8.37 |
| lung normal GW98-165 | 21922 | 30.82 | 16.42 | 32.84 | lung normal | |
| lung tumor GW98-164 | 21921 | 27.45 | 139.52 | 279.04 | lung tumor | 8.50 |
| lung normal GW98-282 | 22584 | 28.47 | 73.15 | 146.30 | lung normal | |
| lung tumor GW98-281 | 22583 | 33.28 | 3.44 | 6.88 | lung tumor | −21.26 |
| breast normal GW00-392 | 28750 | 21.97 | 4550.43 | 4550.43 | breast normal | |
| breast tumor GW00-391 | 28746 | 22.72 | 2838.62 | 5677.24 | breast tumor | 1.25 |
| breast normal GW00-413 | 28798 | 23.12 | 2188.79 | 2188.79 | breast normal | |
| breast tumor GW00-412 | 28797 | 21.4 | 6554.67 | 13109.34 | breast tumor | 5.99 |
| breast normal GW00- | 27592-95 | 25.84 | 390.48 | 390.48 | breast normal | |
| 235:238 | | | | | | |
| breast tumor GW00- | 27588-91 | 20.99 | 8531.75 | 8531.75 | breast tumor | 21.85 |
| 231:234 | | | | | | |
| breast normal GW98-621 | 23656 | 40 | 0 | 0.00 | breast normal | |
| breast tumor GW98-620 | 23655 | 23.15 | 2151.17 | 4302.34 | breast tumor | 4302.34 |
| brain normal BB99-542 | 25507 | 32.3 | 6.4 | 12.80 | brain normal | |
| brain normal BB99-406 | 25509 | 32.95 | 4.23 | 8.46 | brain normal | |
| brain normal BB99-904 | 25546 | 33.67 | 2.68 | 5.36 | brain normal | |
| brain stage 5 ALZ BB99- | 25502 | 33.75 | 2.55 | 5.10 | brain stage 5 | −1.74 |
| 874 | | | | | ALZ | |
| brain stage 5 ALZ BB99- | 25503 | 32.35 | 6.22 | 12.44 | brain stage 5 | 1.40 |
| 887 | | | | | ALZ | |
| brain stage 5 ALZ BB99- | 25504 | 32.23 | 6.71 | 13.42 | brain stage 5 | 1.51 |
| 862 | | | | | ALZ | |
| brain stage 5 ALZ BB99- | 25542 | 32.92 | 4.31 | 8.62 | brain stage 5 | −1.03 |
| 927 | | | | | ALZ | |
| CT lung | normal | 32.56 | 5.44 | 10.88 | CT lung Nml | |
| lung 26 | normal | 33.5 | 2.98 | | lung 26 Nml | |
| lung 27 | normal | 37.3 | 0.27 | 0.27 | lung 27 Nml | |
| lung 24 | COPD | 34.74 | 1.36 | 1.36 | lung 24 COPD | −2.95 |
| lung 28 | COPD | 34.35 | 1.74 | 1.74 | lung 28 COPD | −2.31 |
| lung 23 | COPD | 32.63 | 5.19 | 5.19 | lung 23 COPD | 1.29 |
| lung 25 | normal | 35.4 | 0.89 | 0.89 | lung 25 Nml | |
| asthmatic lung ODO3112 | 29321 | 33.73 | 2.58 | 2.58 | asthmatic lung | −1.56 |
| asthmatic lung ODO3433 | 29323 | 33.44 | 3.1 | 6.20 | asthmatic lung | 1.54 |
| asthmatic lung ODO3397 | 29322 | 33.55 | 2.9 | 5.80 | asthmatic lung | 1.45 |
| asthmatic lung ODO4928 | 29325 | 32.29 | 6.43 | 12.86 | asthmatic lung | 3.20 |
| endo cells | control | 40 | 0.07 | 0.07 | endo cells | |
| endo VEGF | | 40 | 0.11 | 0.11 | endo VEGF | 1.57 |
| endo bFGF | | 40 | 0 | 0.00 | endo bFGF | −0.07 |
| heart Clontech | normal | 36.11 | 0.57 | 1.14 | heart | |
| heart (T-1) ischemic | 29417 | 37.35 | 0.26 | 0.52 | heart (T-1) | −2.19 |
| | | | | | ischemic | |
| heart (T-14) non- | 29422 | 38.7 | 0.11 | 0.22 | heart (T-14) | −5.18 |
| obstructive DCM | | | | | non-obstructive | |
| | | | | | DCM | |
| heart (T-3399) DCM | 29426 | 35.21 | 1.01 | 2.02 | heart (T-3399) | 1.77 |
| | | | | | DCM | |
| adenoid GW99-269 | 26162 | 30.88 | 15.82 | 31.64 | adenoid | |
| tonsil GW98-280 | 22582 | 29.3 | 43.03 | 86.06 | tonsil | |
| T cells PC00314 | 28453 | 33.13 | 3.77 | 7.54 | T cells | |
| PBMNC | | 37.25 | 0.28 | 0.28 | PBMNC | |
| monocyte | | 35.73 | 0.72 | 1.44 | monocyte | |
| B cells PC00665 | 28455 | 31.41 | 11.3 | 22.60 | B cells | |
| dendritic cells | 28441 | 35.45 | 0.86 | 1.72 | dendritic cells | |
| neutrophils | 28440 | 32.96 | 4.2 | 4.20 | neutrophils | |
| eosinophils | 28446 | 36.89 | 0.35 | 0.70 | eosinophils | |
| BM unstim | | 34.15 | 1.98 | 1.98 | BM unstim | |
| BM stim | treated | 34.86 | 1.26 | 1.26 | BM stim | −1.57 |
| osteo dif | treated | 40 | 0.14 | 0.14 | osteo dif | −4.93 |
| osteo undif | | 35.8 | 0.69 | 0.69 | osteo undif | |
| chondrocytes | | 32.84 | 4.54 | 11.35 | chondrocytes | |
| OA Synovium IP12/01 | 29462 | 34.19 | 1.93 | 1.93 | OA Synovium | |
| OA Synovium NP10/01 | 29461 | 34.24 | 1.86 | 3.72 | OA Synovium | |
| OA Synovium NP57/00 | 28464 | 31.49 | 10.71 | 21.42 | OA Synovium | |
| RA Synovium | 28466 | 34.09 | 2.06 | 4.12 | RA Synovium | |
| NP03/01 | | | | | | |
| RA Synovium | 28467 | 32.6 | 5.28 | 10.56 | RA Synovium | |
| NP71/00 | | | | | | |
| RA Synovium | 28475 | 33.57 | 2.86 | 5.72 | RA Synovium | |
| NP45/00 | | | | | | |
| OA bone (biobank) | 29217 | 33.57 | 2.86 | 2.86 | OA bone | |
| | | | | | (biobank) | |
| OA bone Sample 1 | J. Emory | 34.91 | 1.22 | 2.44 | OA bone | |
| OA bone Sample 2 | J. Emory | 32.41 | 5.97 | 11.94 | OA bone | |
| Cartilage (pool) | Normal | 33.4 | 3.18 | 6.36 | Nml Cartilage | |
| | | | | | (pool) | |
| Cartilage (pool) | OA | 34.24 | 1.87 | 3.74 | OA Cartilage | −1.70 |
| | | | | | (pool) | |
| PBL unifected | 28441 | 36.04 | 0.59 | 1.18 | PBL unifected | |
| PBL HIV IIIB | 28442 | 35.1 | 1.08 | 2.16 | PBL HIV IIIB | 1.83 |
| MRC5 uninfected (100%) | 29158 | 36.48 | 0.45 | 0.90 | MRC5 uninfected | |
| | | | | | (100%) | |
| MRC5 HSV strain F | 29178 | 29.9 | 29.43 | 58.86 | MRC5 HSV | 65.40 |
| | | | | | strain F | |
| W12 cells | 29179 | 33.4 | 3.18 | 6.36 | W12 cells | 9.94 |
| Keratinocytes | 29180 | 37.03 | 0.32 | 0.64 | Keratinocytes | |
| B-actin control | | 26.65 | 233.22 | | | |
| genomic | | 23.36 | 1885.22 | | | |
| 1.00E+05 | | 17.13 | 100000 | | | |
| 1.00E+05 | | 17.11 | 100000 | | | |
| 1.00E+04 | | 20.04 | 10000 | | | |
| 1.00E+04 | | 22.87 | 10000 | | | |
| 1.00E+03 | | 23.43 | 1000 | | | |
| 1.00E+03 | | 23.44 | 1000 | | | |
| 1.00E+02 | | 28.05 | 100 | | | |
| 1.00E+02 | | 28.15 | 100 | | | |
| 1.00E+01 | | 31.22 | 10 | | | |
| 1.00E+01 | | 31.21 | 10 | | | |
| 1.00E+00 | | 36.14 | 1 | | | |
| 1.00E+00 | | 40 | 1 | | | |
| NTC | | 40 | 0 | | | |

*lung 26 Normal has been omitted due to multiple amplification failures from that sample

Gene Name gsk1811484BrCaAg

| | Fold Change in Disease |
|---|---|
| | Population Relative to |
| Disease tissues | Normal |
| colon tumor | 85.87 |
| colon tumor | 3.35 |
| colon tumor | 1.70 |
| colon tumor | 6.62 |
| lung tumor | 77.32 |
| lung tumor | −8.37 |
| lung tumor | 8.50 |
| lung tumor | −21.26 |
| breast tumor | 1.25 |
| breast tumor | 5.99 |
| breast tumor | 21.85 |
| breast tumor | 4302.34 |
| brain stage 5 ALZ | −1.74 |
| brain stage 5 ALZ | 1.40 |
| brain stage 5 ALZ | 1.51 |
| brain stage 5 ALZ | −1.03 |
| lung 24 COPD | −2.95 |
| lung 28 COPD | −2.31 |
| lung 23 COPD | 1.29 |
| asthmatic lung | −1.56 |
| asthmatic lung | 1.54 |
| asthmatic lung | 1.45 |
| asthmatic lung | 3.20 |
| endo VEGF | 1.57 |
| endo bFGF | −0.07 |
| heart (T-1) ischemic | −2.19 |
| heart (T-14) non-obstructive DCM | −5.18 |
| heart (T-3399) DCM | 1.77 |
| BM stim | −1.57 |
| osteo dif | −4.93 |
| OA Cartilage (pool) | −1.70 |
| PBL HIV IIIB | 1.83 |
| MRC5 HSV strain F | 65.40 |
| W12 cells | 9.94 |

Gene Name sbg1529984aSproteinase

Moderate to low overall expression. The highest normal expression is seen in the parotid gland. The highest disease expression is seen in one of the colon tumor samples and the neutrophils. Significant upregulation in 1 of 4 colon tumor samples is sufficient to implicate this gene in colon cancer. Downregulation in 2 of 4 lung tumor samples implicates this gene in lung cancer. Upregulation in 1 of 4 Alzheimer's brain samples suggests an involvement in Alzheimer's disease. Downregulation in 3 of 3 disease heart samples implies an involvement in cardiovascular diseases such as non-obstructive and obstructive DCM and ischemia. Upregulated in the stimulated bone marrow. Upregulation in the HSV-infected MRC5 cells and the W12 cells suggests that this gene may be a host factor in HSV and HPV. Patterns of expression are similar to gene sbg1529984bSproteinase

| | | | | | | | copies of |
|---|---|---|---|---|---|---|---|
| | | Mean | Mean | | | | mRNA |
| | | GOI | GOI | | | | detected/ |
| Sample | Ct | copies | copies | Average | 18 S | 50 ng/ | 50 ng |
| sbg1529984a- | (sample | (sample | (sample | GOI | rRNA | 18 S | total |
| Sproteinase | 1 and 2) | 1) | 2) | Copies | (ng) | rRNA(ng) | RNA |
| Subcutaneous | 37.33, 40 | 1.96 | 0 | 0.98 | 3.06 | 16.34 | 16.01 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 32.19, 31.49 | 54.27 | 85.5 | 69.89 | 7.24 | 6.91 | 482.63 |
| Fetal Brain Clontech | 40, 35.86 | 0 | 5.08 | 2.54 | 0.48 | 103.95 | 264.03 |
| Cerebellum Clontech | 36.19, 40 | 4.11 | 0 | 2.06 | 2.17 | 23.04 | 47.35 |
| Cervix | 35.93, 36.94 | 4.85 | 2.52 | 3.69 | 2.42 | 20.66 | 76.14 |
| Colon | 40, 40 | 0 | 0 | 0.00 | 2.71 | 18.45 | 0.00 |
| Endometrium | 36.09, 35.4 | 4.37 | 6.84 | 5.61 | 0.73 | 68.21 | 382.33 |
| Esophagus | 36.74, 36.68 | 2.88 | 2.99 | 2.94 | 1.37 | 36.50 | 107.12 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 36.55, 40 | 3.24 | 0 | 1.62 | 2.58 | 19.38 | 31.40 |
| Jejunum | 33.85, 34.24 | 18.56 | 14.48 | 16.52 | 6.60 | 7.58 | 125.15 |
| Kidney | 40, 40 | 0 | 0.37 | 0.19 | 2.12 | 23.58 | 4.36 |
| Liver | 36.09, 36.8 | 4.37 | 2.77 | 3.57 | 1.50 | 33.33 | 119.00 |
| Fetal Liver Clontech | 35.25, 34.9 | 7.5 | 9.42 | 8.46 | 10.40 | 4.81 | 40.67 |
| Lung | 36.23, 35.33 | 3.99 | 7.16 | 5.58 | 2.57 | 19.46 | 108.46 |
| Mammary Gland | 40, 36.9 | 0 | 2.58 | 1.29 | 13.00 | 3.85 | 4.96 |
| Clontech | | | | | | | |
| Myometrium | 35.51, 40 | 6.34 | 0 | 3.17 | 2.34 | 21.37 | 67.74 |
| Omentum | 34.78, 35.94 | 10.16 | 4.81 | 7.49 | 3.94 | 12.69 | 94.99 |
| Ovary | 34.41, 35.02 | 12.91 | 8.74 | 10.83 | 4.34 | 11.52 | 124.71 |
| Pancreas | 40, 40 | 0.17 | 0.17 | 0.17 | 0.81 | 61.80 | 10.51 |
| Head of Pancreas | 36.03, 40 | 4.55 | 0 | 2.28 | 1.57 | 31.85 | 72.45 |
| Parotid Gland | 28.48, 28.05 | 598.21 | 789.07 | 693.64 | 5.48 | 9.12 | 6328.83 |
| Placenta Clontech | 38.55, 40 | 0.89 | 0.19 | 0.54 | 5.26 | 9.51 | 5.13 |
| Prostate | 40, 40 | 0.15 | 0.09 | 0.12 | 3.00 | 16.67 | 2.00 |
| Rectum | 40, 40 | 0 | 0 | 0.00 | 1.23 | 40.65 | 0.00 |
| Salivary Gland Clontech | 33.68, 33.94 | 20.73 | 17.51 | 19.12 | 7.31 | 6.84 | 130.78 |
| Skeletal Muscle | 40, 40 | 0 | 0 | 0.00 | 1.26 | 39.68 | 0.00 |
| Clontech | | | | | | | |
| Skin | 38.8, 40 | 0.76 | 0 | 0.38 | 1.21 | 41.32 | 15.70 |
| Small Intestine Clontech | 40, 40 | 0 | 0 | 0.00 | 0.98 | 51.07 | 0.00 |
| Spleen | 36.18, 33.96 | 4.13 | 17.37 | 10.75 | 4.92 | 10.16 | 109.25 |
| Stomach | 35.41, 40 | 6.79 | 0.46 | 3.63 | 2.73 | 18.32 | 66.39 |
| Testis Clontech | 40, 36.26 | 0 | 3.91 | 1.96 | 0.57 | 87.87 | 171.79 |
| Thymus Clontech | 34.88, 34.03 | 9.55 | 16.54 | 13.05 | 9.89 | 5.06 | 65.95 |
| Thyroid | 40, 37.19 | 0.09 | 2.14 | 1.12 | 2.77 | 18.05 | 20.13 |
| Trachea Clontech | 35.15, 34.31 | 8.01 | 13.85 | 10.93 | 9.71 | 5.15 | 56.28 |
| Urinary Bladder | 36.62, 40 | 3.11 | 0 | 1.56 | 5.47 | 9.14 | 14.21 |
| Uterus | 34.99, 33.9 | 8.92 | 17.96 | 13.44 | 5.34 | 9.36 | 125.84 |
| genomic | 27.51 | 1122.47 | | | | | |
| b-actin | 28.3 | 670.96 | | | | | |
| 1.00E+05 | 20.54 | 100000 | | | | | |
| 1.00E+05 | 20.43 | 100000 | | | | | |
| 1.00E+04 | 23.84 | 10000 | | | | | |
| 1.00E+04 | 23.89 | 10000 | | | | | |
| 1.00E+03 | 27.26 | 1000 | | | | | |
| 1.00E+03 | 27.32 | 1000 | | | | | |
| 1.00E+02 | 33.08 | 100 | | | | | |
| 1.00E+02 | 31.79 | 100 | | | | | |
| 1.00E+01 | 34.82 | 10 | | | | | |
| 1.00E+01 | 35.25 | 10 | | | | | |
| 1.00E+00 | 38.97 | 1 | | | | | |
| 1.00E+00 | 36.41 | 1 | | | | | |
| NTC | 40 | 0 | | | | | |
| NTC | 36.89 | −1 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1529984a- | (GSK | | GOI | total | | Disease |
| Sproteinase | identifier) | Ct | copies | RNA | Sample | Population |
| colon normal GW98-167 | 21941 | 33.01 | 47.63 | 95.26 | colon normal | |
| colon tumor GW98-166 | 21940 | 33.28 | 40.57 | 81.14 | colon tumor | −1.17 |
| colon normal GW98-178 | 22080 | 34.6 | 18.57 | 37.14 | colon normal | |
| colon tumor GW98-177 | 22060 | 23.82 | 11068.26 | 22136.52 | colon tumor | 596.03 |
| colon normal GW98-561 | 23514 | 33.97 | 27.01 | 54.02 | colon normal | |
| colon tumor GW98-560 | 23513 | 33.95 | 27.3 | 54.60 | colon tumor | 1.01 |
| colon normal GW98-894 | 24691 | 35.44 | 11.27 | 22.54 | colon normal | |
| colon tumor GW98-893 | 24690 | 33.01 | 47.81 | 95.62 | colon tumor | 4.24 |
| lung normal GW98-3 | 20742 | 31.02 | 154.82 | 309.64 | lung normal | |
| lung tumor GW98-2 | 20741 | 39.68 | 0.91 | 1.82 | lung tumor | −170.13 |
| lung normal GW97-179 | 20677 | 32.4 | 68.58 | 137.16 | lung normal | |
| lung tumor GW97-178 | 20676 | 31.5 | 116.75 | 233.50 | lung tumor | 1.70 |
| lung normal GW98-165 | 21922 | 33.82 | 29.54 | 59.08 | lung normal | |
| lung tumor GW98-164 | 21921 | 37.8 | 2.79 | 5.58 | lung tumor | −10.59 |
| lung normal GW98-282 | 22584 | 35.25 | 12.64 | 25.28 | lung normal | |
| lung tumor GW98-281 | 22583 | 35.42 | 11.44 | 22.88 | lung tumor | −1.10 |
| breast normal GW00-392 | 28750 | 33.51 | 35.4 | 35.40 | breast normal | |
| breast tumor GW00-391 | 28746 | 33.59 | 33.83 | 67.66 | breast tumor | 1.91 |
| breast normal GW00-413 | 28798 | 36.84 | 4.93 | 4.93 | breast normal | |
| breast tumor GW00-412 | 28797 | 34.35 | 21.52 | 43.04 | breast tumor | 8.73 |
| breast normal GW00- | 27592-95 | 29.35 | 417.06 | 417.06 | breast normal | |
| 235:238 | | | | | | |
| breast tumor GW00- | 27588-91 | 34.13 | 24.49 | 24.49 | breast tumor | −17.03 |
| 231:234 | | | | | | |
| breast normal GW98-621 | 23656 | 32.79 | 54.35 | 108.70 | breast normal | |
| breast tumor GW98-620 | 23655 | 35.17 | 13.28 | 26.56 | breast tumor | −4.09 |
| brain normal BB99-542 | 25507 | 33.46 | 36.5 | 73.00 | brain normal | |
| brain normal BB99-406 | 25509 | 32.74 | 55.8 | 111.60 | brain normal | |
| brain normal BB99-904 | 25546 | 34.54 | 19.21 | 38.42 | brain normal | |
| brain stage 5 ALZ BB99- | 25502 | 32.76 | 55.46 | 110.92 | brain stage 5 ALZ | 1.49 |
| 874 | | | | | | |
| brain stage 5 ALZ BB99- | 25503 | 30.49 | 212.89 | 425.78 | brain stage 5 ALZ | 5.73 |
| 887 | | | | | | |
| brain stage 5 ALZ BB99- | 25504 | 32.66 | 58.74 | 117.48 | brain stage 5 ALZ | 1.58 |
| 862 | | | | | | |
| brain stage 5 ALZ BB99- | 25542 | 32.34 | 70.96 | 141.92 | brain stage 5 ALZ | 1.91 |
| 927 | | | | | | |
| CT lung | normal | 32.01 | 86.34 | 172.68 | CT lung Nml | |
| lung 26 | normal | 33.37 | 38.62 | | lung 26 Nml | |
| lung 27 | normal | 34.67 | 17.88 | 17.88 | lung 27 Nml | |
| lung 24 | COPD | 36.41 | 6.38 | 6.38 | lung 24 COPD | −15.13 |
| lung 28 | COPD | 38.31 | 2.07 | 2.07 | lung 28 COPD | −46.64 |
| lung 23 | COPD | 35.21 | 12.97 | 12.97 | lung 23 COPD | −7.44 |
| lung 25 | normal | 31.78 | 99.07 | 99.07 | lung 25 Nml | |
| asthmatic lung ODO3112 | 29321 | 32.79 | 54.41 | 54.41 | asthmatic lung | −1.77 |
| asthmatic lung ODO3433 | 29323 | 32.38 | 69.2 | 138.40 | asthmatic lung | 1.43 |
| asthmatic lung ODO3397 | 29322 | 33.12 | 44.79 | 89.58 | asthmatic lung | −1.08 |
| asthmatic lung ODO4928 | 29325 | 34.02 | 26.23 | 52.46 | asthmatic lung | −1.84 |
| endo cells | control | 36.04 | 7.91 | 7.91 | endo cells | |
| endo VEGF | | 40 | 0 | 0.00 | endo VEGF | −7.91 |
| endo bFGF | | 40 | 0 | 0.00 | endo bFGF | −7.91 |
| heart Clontech | normal | 33.23 | 41.97 | 83.94 | heart | |
| heart (T-1) ischemic | 29417 | 37.79 | 2.8 | 5.60 | heart (T-1) | −14.99 |
| | | | | | ischemic | |
| heart (T-14) non- | 29422 | 40 | 0 | 0.00 | heart (T-14) non- | −83.94 |
| obstructive DCM | | | | | obstructive DCM | |
| heart (T-3399) DCM | 29426 | 40 | 0.36 | 0.72 | heart (T-3399) | −116.58 |
| | | | | | DCM | |
| adenoid GW99-269 | 26162 | 34.46 | 20.24 | 40.48 | adenoid | |
| tonsil GW98-280 | 22582 | 33.25 | 41.48 | 82.96 | tonsil | |
| T cells PC00314 | 28453 | 31.93 | 90.58 | 181.16 | T cells | |
| PBMNC | | 34.14 | 24.45 | 24.45 | PBMNC | |
| monocyte | | 32.46 | 66.01 | 132.02 | monocyte | |
| B cells PC00665 | 28455 | 32.97 | 48.75 | 97.50 | B cells | |
| dendritic cells | 28441 | 34.32 | 21.93 | 43.86 | dendritic cells | |
| neutrophils | 28440 | 22.25 | 27946.82 | 27946.82 | neutrophils | |
| eosinophils | 28446 | 28.14 | 851.97 | 1703.94 | eosinophils | |
| BM unstim | | 33.69 | 31.96 | 31.96 | BM unstim | |
| BM stim | treated | 30.89 | 167.46 | 167.46 | BM stim | 5.24 |
| osteo dif | treated | 40 | 0 | 0.00 | osteo dif | 0.00 |
| osteo undif | | 40 | 0 | 0.00 | osteo undif | |
| chondrocytes | | 34.76 | 16.86 | 42.15 | chondrocytes | |
| OA Synovium IP12/01 | 29462 | 34.21 | 23.4 | 23.40 | OA Synovium | |
| OA Synovium NP10/01 | 29461 | 34.29 | 22.39 | 44.78 | OA Synovium | |
| OA Synovium NP57/00 | 28464 | 34.26 | 22.69 | 45.38 | OA Synovium | |
| RA Synovium | 28466 | 37.26 | 3.85 | 7.70 | RA Synovium | |
| NP03/01 | | | | | | |
| RA Synovium | 28467 | 34.61 | 18.51 | 37.02 | RA Synovium | |
| NP71/00 | | | | | | |
| RA Synovium | 28475 | 35.47 | 11.11 | 22.22 | RA Synovium | |
| NP45/00 | | | | | | |
| OA bone (biobank) | 29217 | 29.87 | 307.3 | 307.30 | OA bone (biobank) | |
| OA bone Sample 1 | J. Emory | 34.01 | 26.31 | 52.62 | OA bone | |
| OA bone Sample 2 | J. Emory | 31.13 | 145.42 | 290.84 | OA bone | |
| Cartilage (pool) | Normal | 32.76 | 55.43 | 110.86 | Nml Cartilage | |
| | | | | | (pool) | |
| Cartilage (pool) | OA | 34.04 | 25.93 | 51.86 | OA Cartilage (pool) | −2.14 |
| PBL unifected | 28441 | 32.49 | 65.05 | 130.10 | PBL unifected | |
| PBL HIV IIIB | 28442 | 33.92 | 27.87 | 55.74 | PBL HIV IIIB | −2.33 |
| MRC5 uninfected | 29158 | 40 | 0.38 | 0.76 | MRC5 uninfected | |
| (100%) | | | | | (100%) | |
| MRC5 HSV strain F | 29178 | 31.18 | 141.21 | 282.42 | MRC5 HSV strain | 371.61 |
| | | | | | F | |
| W12 cells | 29179 | 36.9 | 4.77 | 9.54 | W12 cells | 16.45 |
| Keratinocytes | 29180 | 40 | 0.29 | 0.58 | Keratinocytes | |
| B-actin control | | 27.78 | 1057.73 | | | |
| genomic | | 27.25 | 1446.89 | | | |
| 1.00E+05 | | 20.4 | 100000 | | | |
| 1.00E+05 | | 20.39 | 100000 | | | |
| 1.00E+04 | | 23.91 | 10000 | | | |
| 1.00E+04 | | 23.82 | 10000 | | | |
| 1.00E+03 | | 27.52 | 1000 | | | |
| 1.00E+03 | | 27.36 | 1000 | | | |
| 1.00E+02 | | 32.51 | 100 | | | |
| 1.00E+02 | | 31.18 | 100 | | | |
| 1.00E+01 | | 36.29 | 10 | | | |
| 1.00E+01 | | 35.38 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name sbg1529984aSproteinase

| | Fold Change in Disease |
|---|---|
| | Population Relative to |
| Disease tissues | Normal |
| colon tumor | −1.17 |
| colon tumor | 596.03 |
| colon tumor | 1.01 |
| colon tumor | 4.24 |
| lung tumor | −170.13 |
| lung tumor | 1.70 |
| lung tumor | −10.59 |
| lung tumor | −1.10 |
| breast tumor | 1.91 |
| breast tumor | 8.73 |
| breast tumor | −17.03 |
| breast tumor | −4.09 |
| brain stage 5 ALZ | 1.49 |
| brain stage 5 ALZ | 5.73 |
| brain stage 5 ALZ | 1.58 |
| brain stage 5 ALZ | 1.91 |
| lung 24 COPD | −15.13 |
| lung 28 COPD | −46.64 |
| lung 23 COPD | −7.44 |
| asthmatic lung | −1.77 |
| asthmatic lung | 1.43 |
| asthmatic lung | −1.08 |
| asthmatic lung | −1.84 |
| endo VEGF | −7.91 |
| endo bFGF | −7.91 |
| heart (T-1) ischemic | −14.99 |
| heart (T-14) non-obstructive DCM | −83.94 |
| heart (T-3399) DCM | −116.58 |
| BM stim | 5.24 |
| osteo dif | 0.00 |
| OA Cartilage (pool) | −2.14 |
| PBL HIV IIIB | −2.33 |
| MRC5 HSV strain F | 371.61 |
| W12 cells | 16.45 |

Gene Name sbg1529984bSproteinase

Moderate to low overall expression. The highest normal expression is seen in the parotid gland. The highest disease expression is seen in one of the colon tumor samples and the neutrophils. Upregulation in 1 of 4 Alzheimer's brain samples suggests an involvement in Alzheimer's disease. Upregulation in 1 of 4 asthmatic lung samples with corroborating high expression in the neutrophils indicates a possible involvement in asthma. Downregulation in 3 of 3 disease heart samples implies an involvement in cardiovascular diseases such as non-obstructive and obstructive DCM and ischemia. Downregulated in the OA cartilage pool may indicate some involvement in osteoarthritis. Upregulation in the HSV-infected MRC5 cells and the W12 cells suggests that this gene may be a host factor in HSV and HPV. Patterns of expression are similar to gene sbg1529984aSproteinase.

| | | | | | | | copies of |
|---|---|---|---|---|---|---|---|
| | | Mean | Mean | | | | mRNA |
| | | GOI | GOI | | | | detected/ |
| Sample | Ct | copies | copies | Average | 18 S | 50 ng/ | 50 ng |
| sbg1529984b- | (sample | (sample | (sample | GOI | rRNA | 18 S | total |
| Sproteinase | 1 and 2) | 1) | 2) | Copies | (ng) | rRNA(ng) | RNA |
| Subcutaneous | 40, 40 | 0.47 | 0 | 0.24 | 3.06 | 16.34 | 3.84 |
| Adipocytes Zenbio | | | | | | | |
| Subcutaneous Adipose | 40, 40 | 0.26 | 0.27 | 0.27 | 0.96 | 52.36 | 13.87 |
| Zenbio | | | | | | | |
| Adrenal Gland Clontech | 37, 40 | 2.31 | 0 | 1.16 | 0.61 | 81.97 | 94.67 |
| Whole Brain Clontech | 32.52, 33.06 | 51.17 | 35.24 | 43.21 | 7.24 | 6.91 | 298.38 |
| Fetal Brain Clontech | 37.12, 39.18 | 2.12 | 0.51 | 1.32 | 0.48 | 103.95 | 136.69 |
| Cerebellum Clontech | 35.29, 37.27 | 7.52 | 1.92 | 4.72 | 2.17 | 23.04 | 108.76 |
| Cervix | 36.82, 36.06 | 2.63 | 4.42 | 3.53 | 2.42 | 20.66 | 72.83 |
| Colon | 38.68, 36.4 | 0.72 | 3.5 | 2.11 | 2.71 | 18.45 | 38.93 |
| Endometrium | 39.44, 36.13 | 0.43 | 4.22 | 2.33 | 0.73 | 68.21 | 158.59 |
| Esophagus | 40, 40 | 0 | 0.3 | 0.15 | 1.37 | 36.50 | 5.47 |
| Heart Clontech | 40, 40 | 0 | 0 | 0.00 | 1.32 | 37.88 | 0.00 |
| Hypothalamus | 40, 40 | 0.28 | 0 | 0.14 | 0.32 | 155.28 | 21.74 |
| Ileum | 37.22, 40 | 1.99 | 0 | 1.00 | 2.58 | 19.38 | 19.28 |
| Jejunum | 34.02, 33.08 | 18.08 | 34.83 | 26.46 | 6.60 | 7.58 | 200.42 |
| Kidney | 40, 40 | 0 | 0 | 0.00 | 2.12 | 23.58 | 0.00 |
| Liver | 34.3, 35.14 | 14.98 | 8.35 | 11.67 | 1.50 | 33.33 | 388.83 |
| Fetal Liver Clontech | 35.03, 37.36 | 9.03 | 1.8 | 5.42 | 10.40 | 4.81 | 26.03 |
| Lung | 40, 40 | 0 | 0.57 | 0.29 | 2.57 | 19.46 | 5.54 |
| Mammary Gland | 38.73, 35.64 | 0.7 | 5.92 | 3.31 | 13.00 | 3.85 | 12.73 |
| Clontech - | | | | | | | |
| Myometrium | 35.44, 37.27 | 6.79 | 1.92 | 4.36 | 2.34 | 21.37 | 93.06 |
| Omentum | 34.27, 35.21 | 15.24 | 7.99 | 11.62 | 3.94 | 12.69 | 147.40 |
| Ovary | 35.03, 34.35 | 9.05 | 14.47 | 11.76 | 4.34 | 11.52 | 135.48 |
| Pancreas | 40, 40 | 0 | 0 | 0.00 | 0.81 | 61.80 | 0.00 |
| Head of Pancreas | 40, 36.77 | 0 | 2.72 | 1.36 | 1.57 | 31.85 | 43.31 |
| Parotid Gland | 28.68, 28.62 | 725.93 | 756.36 | 741.15 | 5.48 | 9.12 | 6762.27 |
| Placenta Clontech | 40, 40 | 0 | 0 | 0.00 | 5.26 | 9.51 | 0.00 |
| Prostate | 40, 40 | 0 | 0 | 0.00 | 3.00 | 16.67 | 0.00 |
| Rectum | 40, 40 | 1.34 | 0 | 0.67 | 1.23 | 40.65 | 27.24 |
| Salivary Gland Clontech | 34.62, 34.03 | 11.98 | 18.05 | 15.02 | 7.31 | 6.84 | 102.70 |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 35.91, 35.58 | 4.91 | 6.15 | 5.53 | 4.92 | 10.16 | 56.20 |
| Stomach | 37.76, 40 | 1.37 | 0.66 | 1.02 | 2.73 | 18.32 | 18.59 |
| Testis Clontech | 35.61, 40 | 6.04 | 0 | 3.02 | 0.57 | 87.87 | 265.38 |
| Thymus Clontech | 36.63, 34.62 | 2.99 | 11.94 | 7.47 | 9.89 | 5.06 | 37.74 |
| Thyroid | 40, 40 | 0 | 0 | 0.00 | 2.77 | 18.05 | 0.00 |
| Trachea Clontech | 36.01, 35.55 | 4.59 | 6.32 | 5.46 | 9.71 | 5.15 | 28.09 |
| Urinary Bladder | 40, 40 | 0 | 0 | 0.00 | 5.47 | 9.14 | 0.00 |
| Uterus | 33.41, 34.08 | 27.65 | 17.46 | 22.56 | 5.34 | 9.36 | 211.19 |
| genomic | 27.92 | 1231.29 | | | | | |
| b-actin | 28.22 | 998.03 | | | | | |
| 1.00E+05 | 21.3 | 100000 | | | | | |
| 1.00E+05 | 21.29 | 100000 | | | | | |
| 1.00E+04 | 24.68 | 10000 | | | | | |
| 1.00E+04 | 24.78 | 10000 | | | | | |
| 1.00E+03 | 28.22 | 1000 | | | | | |
| 1.00E+03 | 28.21 | 1000 | | | | | |
| 1.00E+02 | 32.66 | 100 | | | | | |
| 1.00E+02 | 32.4 | 100 | | | | | |
| 1.00E+01 | 40 | 10 | | | | | |
| 1.00E+01 | 34.9 | 10 | | | | | |
| 1.00E+00 | 37.09 | 1 | | | | | |
| 1.00E+00 | 40 | 0 | | | | | |
| NTC | 40 | −1 | | | | | |
| NTC | 40 | −1 | | | | | |

| | | | | copies of | | |
|---|---|---|---|---|---|---|
| | | | | mRNA | | |
| | Reg | | | detected/ | | Fold |
| Sample | number | | Mean | 50 ng | | Change in |
| sbg1529984b- | (GSK | | GOI | total | | Disease |
| Sproteinase | identifier) | Ct | copies | RNA | Sample | Population |
| colon normal GW98-167 | 21941 | 33.89 | 30.45 | 60.90 | colon normal | |
| colon tumor GW98-166 | 21940 | 33.04 | 50.18 | 100.36 | colon tumor | 1.65 |
| colon normal GW98-178 | 22080 | 40 | 0.43 | 0.86 | colon normal | |
| colon tumor GW98-177 | 22060 | 23.3 | 15255.15 | 30510.30 | colon tumor | 35477.09 |
| colon normal GW98-561 | 23514 | 33.79 | 32.34 | 64.68 | colon normal | |
| colon tumor GW98-560 | 23513 | 35.14 | 14.59 | 29.18 | colon tumor | −2.22 |
| colon normal GW98-894 | 24691 | 35.74 | 10.28 | 20.56 | colon normal | |
| colon tumor GW98-893 | 24690 | 33.32 | 42.65 | 85.30 | colon tumor | 4.15 |
| lung normal GW98-3 | 20742 | 32.85 | 55.9 | 111.80 | lung normal | |
| lung tumor GW98-2 | 20741 | 36.17 | 7.98 | 15.96 | lung tumor | −7.01 |
| lung normal GW97-179 | 20677 | 33.46 | 39.17 | 78.34 | lung normal | |
| lung tumor GW97-178 | 20676 | 33.28 | 43.58 | 87.16 | lung tumor | 1.11 |
| lung normal GW98-165 | 21922 | 33.39 | 40.91 | 81.82 | lung normal | |
| lung tumor GW98-164 | 21921 | 35.46 | 12.1 | 24.20 | lung tumor | −3.38 |
| lung normal GW98-282 | 22584 | 35.86 | 9.56 | 19.12 | lung normal | |
| lung tumor GW98-281 | 22583 | 33.28 | 43.49 | 86.98 | lung tumor | 4.55 |
| breast normal GW00-392 | 28750 | 33.88 | 30.69 | 30.69 | breast normal | |
| breast tumor GW00-391 | 28746 | 33.87 | 30.88 | 61.76 | breast tumor | 2.01 |
| breast normal GW00-413 | 28798 | 34.84 | 17.43 | 17.43 | breast normal | |
| breast tumor GW00-412 | 28797 | 33.43 | 39.9 | 79.80 | breast tumor | 4.58 |
| breast normal GW00- | 27592-95 | 31.31 | 138.23 | 138.23 | breast normal | |
| 235:238 | | | | | | |
| breast tumor GW00- | 27588-91 | 35.73 | 10.32 | 10.32 | breast tumor | −13.39 |
| 231:234 | | | | | | |
| breast normal GW98-621 | 23656 | 33.38 | 41.17 | 82.34 | breast normal | |
| breast tumor GW98-620 | 23655 | 37.03 | 4.82 | 9.64 | breast tumor | −8.54 |
| brain normal BB99-542 | 25507 | 33.79 | 32.3 | 64.60 | brain normal | |
| brain normal BB99-406 | 25509 | 33.02 | 50.66 | 101.32 | brain normal | |
| brain normal BB99-904 | 25546 | 36.01 | 8.79 | 17.58 | brain normal | |
| brain stage 5 ALZ BB99- | 25502 | 34.82 | 17.69 | 35.38 | brain stage 5 ALZ | −1.73 |
| 874 | | | | | | |
| brain stage 5 ALZ BB99- | 25503 | 31.51 | 123.39 | 246.78 | brain stage 5 ALZ | 4.03 |
| 887 | | | | | | |
| brain stage 5 ALZ BB99- | 25504 | 33.36 | 41.66 | 83.32 | brain stage 5 ALZ | 1.36 |
| 862 | | | | | | |
| brain stage 5 ALZ BB99- | 25542 | 34.27 | 24.35 | 48.70 | brain stage 5 ALZ | −1.26 |
| 927 | | | | | | |
| CT lung | normal | 34.69 | 19.01 | 38.02 | CT lung Nml | |
| lung 26 | normal | 36.24 | 7.66 | | lung 26 Nml | |
| lung 27 | normal | 34.72 | 18.68 | 18.68 | lung 27 Nml | |
| lung 24 | COPD | 35.99 | 8.88 | 8.88 | lung 24 COPD | −2.43 |
| lung 28 | COPD | 40 | 0 | 0.00 | lung 28 COPD | −21.54 |
| lung 23 | COPD | 36.03 | 8.66 | 8.66 | lung 23 COPD | −2.49 |
| lung 25 | normal | 36.19 | 7.92 | 7.92 | lung 25 Nml | |
| asthmatic lung ODO3112 | 29321 | 35.14 | 14.64 | 14.64 | asthmatic lung | −1.47 |
| asthmatic lung ODO3433 | 29323 | 31.97 | 93.96 | 187.92 | asthmatic lung | 8.72 |
| asthmatic lung ODO3397 | 29322 | 35.36 | 12.86 | 25.72 | asthmatic lung | 1.19 |
| asthmatic lung ODO4928 | 29325 | 34.06 | 27.62 | 55.24 | asthmatic lung | 2.56 |
| endo cells | control | 38.71 | 1.8 | 1.80 | endo cells | |
| endo VEGF | | 40 | 0 | 0.00 | endo VEGF | −1.80 |
| endo bFGF | | 37.98 | 2.77 | 2.77 | endo bFGF | 1.54 |
| heart Clontech | normal | 36.01 | 8.8 | 17.60 | heart | |
| heart (T-1) ischemic | 29417 | 40 | 0 | 0.00 | heart (T-1) | −17.60 |
| | | | | | ischemic | |
| heart (T-14) non- | 29422 | 40 | 0.47 | 0.94 | heart (T-14) non- | −18.72 |
| obstructive DCM | | | | | obstructive DCM | |
| heart (T-3399) DCM | 29426 | 40 | 0 | 0.00 | heart (T-3399) | −17.60 |
| | | | | | DCM | |
| adenoid GW99-269 | 26162 | 36.45 | 6.76 | 13.52 | adenoid | |
| tonsil GW98-280 | 22582 | 34.4 | 22.64 | 45.28 | tonsil | |
| T cells PC00314 | 28453 | 32.3 | 77.45 | 154.90 | T cells | |
| PBMNC | | 34.29 | 24.11 | 24.11 | PBMNC | |
| monocyte | | 31.98 | 93.46 | 186.92 | monocyte | |
| B cells PC00665 | 28455 | 32.58 | 65.73 | 131.46 | B cells | |
| dendritic cells | 28441 | 37.09 | 4.65 | 9.30 | dendritic cells | |
| neutrophils | 28440 | 23.91 | 10649.45 | 10649.45 | neutrophils | |
| eosinophils | 28446 | 30.49 | 223.9 | 447.80 | eosinophils | |
| BM unstim | | 33.78 | 32.43 | 32.43 | BM unstim | |
| BM stim | treated | 32.38 | 73.77 | 73.77 | BM stim | 2.27 |
| osteo dif | treated | 40 | 0 | 0.00 | osteo dif | 0.00 |
| osteo undif | | 40 | 0 | 0.00 | osteo undif | |
| chondrocytes | | 34.82 | 17.68 | 44.20 | chondrocytes | |
| OA Synovium IP12/01 | 29462 | 36.98 | 4.97 | 4.97 | OA Synovium | |
| OA Synovium NP10/01 | 29461 | 35.2 | 14.13 | 28.26 | OA Synovium | |
| OA Synovium NP57/00 | 28464 | 34.26 | 24.5 | 49.00 | OA Synovium | |
| RA Synovium | 28466 | 40 | 0 | 0.00 | RA Synovium | |
| NP03/01 | | | | | | |
| RA Synovium | 28467 | 35.38 | 12.73 | 25.46 | RA Synovium | |
| NP71/00 | | | | | | |
| RA Synovium | 28475 | 36.77 | 5.62 | 11.24 | RA Synovium | |
| NP45/00 | | | | | | |
| OA bone (biobank) | 29217 | 31.73 | 108 | 108.00 | OA bone | |
| | | | | | (biobank) | |
| OA bone Sample 1 | J. Emory | 35.18 | 14.25 | 28.50 | OA bone | |
| OA bone Sample 2 | J. Emory | 31.39 | 132.09 | 264.18 | OA bone | |
| Cartilage (pool) | Normal | 34.99 | 15.95 | 31.90 | Nml Cartilage | |
| | | | | | (pool) | |
| Cartilage (pool) | OA | 40 | 0.46 | 0.92 | OA Cartilage | −34.67 |
| | | | | | (pool) | |
| PBL unifected | 28441 | 34.62 | 19.9 | 39.80 | PBL unifected | |
| PBL HIV IIIB | 28442 | 34.61 | 19.96 | 39.92 | PBL HIV IIIB | 1.00 |
| MRC5 uninfected | 29158 | 36.25 | 7.61 | 15.22 | MRC5 uninfected | |
| (100%) | | | | | (100%) | |
| MRC5 HSV strain F | 29178 | 31.57 | 118.66 | 237.32 | MRC5 HSV strain | 15.59 |
| | | | | | F | |
| W12 cells | 29179 | 35.02 | 15.7 | 31.40 | W12 cells | 3.37 |
| Keratinocytes | 29180 | 37.09 | 4.66 | 9.32 | Keratinocytes | |
| B-actin control | | 27.82 | 1072.85 | | | |
| genomic | | 27.31 | 1449.65 | | | |
| 1.00E+05 | | 20.38 | 100000 | | | |
| 1.00E+05 | | 20.29 | 100000 | | | |
| 1.00E+04 | | 23.77 | 10000 | | | |
| 1.00E+04 | | 23.81 | 10000 | | | |
| 1.00E+03 | | 27.41 | 1000 | | | |
| 1.00E+03 | | 27.4 | 1000 | | | |
| 1.00E+02 | | 31.71 | 100 | | | |
| 1.00E+02 | | 33.6 | 100 | | | |
| 1.00E+01 | | 35.52 | 10 | | | |
| 1.00E+01 | | 35.51 | 10 | | | |
| 1.00E+00 | | 40 | 0 | | | |
| 1.00E+00 | | 40 | 1 | | | |
| NTC | | 40 | 0 | | | |

*lung 26 Normal has been omitted due to multiple amplification failures from that sample

Gene Name sbg1529984bSproteinase

| | Fold Change in Disease |
|---|---|
| | Population Relative to |
| Disease tissues | Normal |
| colon tumor | 1.65 |
| colon tumor | 35477.09 |
| colon tumor | −2.22 |
| colon tumor | 4.15 |
| lung tumor | −7.01 |
| lung tumor | 1.11 |
| lung tumor | −3.38 |
| lung tumor | 4.55 |
| breast tumor | 2.01 |
| breast tumor | 4.58 |
| breast tumor | −13.39 |
| breast tumor | −8.54 |
| brain stage 5 ALZ | −1.73 |
| brain stage 5 ALZ | 4.03 |
| brain stage 5 ALZ | 1.36 |
| brain stage 5 ALZ | −1.26 |
| lung 24 COPD | −2.43 |
| lung 28 COPD | −21.54 |
| lung 23 COPD | −2.49 |
| asthmatic lung | −1.47 |
| asthmatic lung | 8.72 |
| asthmatic lung | 1.19 |
| asthmatic lung | 2.56 |
| endo VEGF | −1.80 |
| endo bFGF | 1.54 |
| heart (T-1) ischemic | −17.60 |
| heart (T-14) non-obstructive DCM | −18.72 |
| heart (T-3399) DCM | −17.60 |
| BM stim | 2.27 |
| osteo dif | 0.00 |
| OA Cartilage (pool) | −34.67 |
| PBL HIV IIIB | 1.00 |
| MRC5 HSV strain F | 15.59 |
| W12 cells | 3.37 |

Gene Name SBGKIN113 (Taqman was Performed for this Sample)

Moderate to low overall expression. The highest normal expression is seen in the omentum and in most of the samples representing the female reproductive system including the cervix, endometrium, and uterus. The highest disease expression is seen in the Alzheimer's brain samples and in the HSV-infected MRC5 cells. Significant upregulation in 1 of 4 colon tumor samples is sufficient to implicate this gene in colon cancer. Upregulation in 2 of 3 COPD lung samples and in 2 of 4 asthmatic lung samples with corroborating high expression in T cells and neutrophils implicates this gene in COPD and asthma. Upregulation in 3 of 3 disease heart samples implies an involvement in cardiovascular diseases such as ischemic, non-obstructive, and obstructive DCM. Downregulated in the stimulated bone marrow and the differentiated osteoblasts. Downregulation in the HIV-infected PBLs suggests that this gene may be a host factor in HIV. Upregulation in the HSV-infected MRC5 cells and the W12 cells suggests that this gene may be a host factor in HSV and HPV.

| | | | | | | | copies of |
|---|---|---|---|---|---|---|---|
| | | Mean | Mean | | | | mRNA |
| | | GOI | GOI | | | 50 | detected/ |
| | Ct | copies | copies | Average | 18 S | ng/18 S | 50 ng |
| Sample | (sample | (sample | (sample | GOI | rRNA | rRNA | total |
| SBGKIN113 | 1 and 2) | 1) | 2) | Copies | (ng) | (ng) | RNA |
| Subcutaneous | 37.77, 40 | 4.66 | 0 | 2.33 | 3.06 | 16.34 | 38.07 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 33.57, 33.2 | 60.28 | 75.81 | 68.05 | 7.24 | 6.91 | 469.92 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 34.88, 35.29 | 27.11 | 21.13 | 24.12 | 2.42 | 20.66 | 498.35 |
| Colon | 35.74, 37.61 | 16.1 | 5.13 | 10.62 | 2.71 | 18.45 | 195.85 |
| Endometrium | 40, 34.98 | 0 | 25.58 | 12.79 | 0.73 | 68.21 | 872.44 |
| Esophagus | 40, 36.42 | 0 | 10.58 | 5.29 | 1.37 | 36.50 | 193.07 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 37.83, 37.07 | 4.49 | 7.14 | 5.82 | 2.58 | 19.38 | 112.69 |
| Jejunum | 34.45, 33.69 | 35.4 | 56.12 | 45.76 | 6.60 | 7.58 | 346.67 |
| Kidney | 40, 37.19 | 0 | 6.62 | 3.31 | 2.12 | 23.58 | 78.07 |
| Liver | 40, 35.52 | 0 | 18.39 | 9.20 | 1.50 | 33.33 | 306.50 |
| Fetal Liver Clontech | 37.47, 37.04 | 5.58 | 7.28 | 6.43 | 10.40 | 4.81 | 30.91 |
| Lung | 37.45, 40 | 5.67 | 0 | 2.84 | 2.57 | 19.46 | 55.16 |
| Mammary Gland | 37.32, 40 | 6.13 | 0 | 3.07 | 13.00 | 3.85 | 11.79 |
| Clontech | | | | | | | |
| Myometrium | 40, 37.01 | 0 | 7.37 | 3.69 | 2.34 | 21.37 | 78.74 |
| Omentum | 33.1, 31.81 | 80.47 | 176.36 | 128.42 | 3.94 | 12.69 | 1629.63 |
| Ovary | 35.21, 34.19 | 22.16 | 41.28 | 31.72 | 4.34 | 11.52 | 365.44 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 35.26, 35.39 | 21.54 | 19.94 | 20.74 | 5.48 | 9.12 | 189.23 |
| Placenta Clontech | 36.42, 38.21 | 10.61 | 3.55 | 7.08 | 5.26 | 9.51 | 67.30 |
| Prostate | 37.83, 40 | 4.48 | 0 | 2.24 | 3.00 | 16.67 | 37.33 |
| Rectum | 40, 38.37 | 0 | 3.22 | 1.61 | 1.23 | 40.65 | 65.45 |
| Salivary Gland Clontech | 40, 40 | 0 | 0 | 0.00 | 7.31 | 6.84 | 0.00 |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine Clontech | 40, 40 | 0 | 0 | 0.00 | 0.98 | 51.07 | 0.00 |
| Spleen | 34.9, 34.62 | 26.86 | 31.82 | 29.34 | 4.92 | 10.16 | 298.17 |
| Stomach | 36.03, 40 | 13.42 | 0 | 6.71 | 2.73 | 18.32 | 122.89 |
| Testis Clontech | 35.78, 40 | 15.69 | 0 | 7.85 | 0.57 | 87.87 | 689.37 |
| Thymus Clontech | 33.25, 33.07 | 73.31 | 81.91 | 77.61 | 9.89 | 5.06 | 392.37 |
| Thyroid | 40, 40 | 0 | 0 | 0.00 | 2.77 | 18.05 | 0.00 |
| Trachea Clontech | 40, 40 | 0 | 0 | 0.00 | 9.71 | 5.15 | 0.00 |
| Urinary Bladder | 37.79, 40 | 4.58 | 0 | 2.29 | 5.47 | 9.14 | 20.93 |
| Uterus | 33.77, 31.89 | 53.34 | 168.09 | 110.72 | 5.34 | 9.36 | 1036.66 |
| genomic | 28.95 | 1012.33 | | | | | |
| b-actin | 29.43 | 755.05 | | | | | |
| 1.00E+05 | 21.37 | 100000 | | | | | |
| 1.00E+05 | 21.69 | 100000 | | | | | |
| 1.00E+04 | 25.17 | 10000 | | | | | |
| 1.00E+04 | 25.26 | 10000 | | | | | |
| 1.00E+03 | 28.66 | 1000 | | | | | |
| 1.00E+03 | 28.69 | 1000 | | | | | |
| 1.00E+02 | 32.6 | 100 | | | | | |
| 1.00E+02 | 33.12 | 100 | | | | | |
| 1.00E+01 | 36.63 | 10 | | | | | |
| 1.00E+01 | 40 | 0 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| SBGKIN113 | identifier) | Ct | copies | RNA | Sample | Population |
| colon normal GW98-167 | 21941 | 32.29 | 99.44 | 198.88 | colon normal | |
| colon tumor GW98-166 | 21940 | 36 | 11.3 | 22.60 | colon tumor | −8.80 |
| colon normal GW98-178 | 22080 | 33.39 | 51.93 | 103.86 | colon normal | |
| colon tumor GW98-177 | 22060 | 31.89 | 125.72 | 251.44 | colon tumor | 2.42 |
| colon normal GW98-561 | 23514 | 40 | 0 | 0.00 | colon normal | |
| colon tumor GW98-560 | 23513 | 37.02 | 6.22 | 12.44 | colon tumor | 12.44 |
| colon normal GW98-894 | 24691 | 35.26 | 17.38 | 34.76 | colon normal | |
| colon tumor GW98-893 | 24690 | 36.25 | 9.72 | 19.44 | colon tumor | −1.79 |
| lung normal GW98-3 | 20742 | 33.39 | 52.16 | 104.32 | lung normal | |
| lung tumor GW98-2 | 20741 | 33.74 | 42.52 | 85.04 | lung tumor | −1.23 |
| lung normal GW97-179 | 20677 | 34.21 | 32.18 | 64.36 | lung normal | |
| lung tumor GW97-178 | 20676 | 33.04 | 64 | 128.00 | lung tumor | 1.99 |
| lung normal GW98-165 | 21922 | 34.22 | 32 | 64.00 | lung normal | |
| lung tumor GW98-164 | 21921 | 31.95 | 121.31 | 242.62 | lung tumor | 3.79 |
| lung normal GW98-282 | 22584 | 33.02 | 64.86 | 129.72 | lung normal | |
| lung tumor GW98-281 | 22583 | 35.84 | 12.4 | 24.80 | lung tumor | −5.23 |
| breast normal GW00-392 | 28750 | 32.93 | 68.09 | 68.09 | breast normal | |
| breast tumor GW00-391 | 28746 | 34.08 | 34.82 | 69.64 | breast tumor | 1.02 |
| breast normal GW00-413 | 28798 | 33.61 | 45.76 | 45.76 | breast normal | |
| breast tumor GW00-412 | 28797 | 33.13 | 60.66 | 121.32 | breast tumor | 2.65 |
| breast normal GW00- | 27592-95 | 33.26 | 56.26 | 56.26 | breast normal | |
| 235:238 | | | | | | |
| breast tumor GW00- | 27588-91 | 34.16 | 33.24 | 33.24 | breast tumor | −1.69 |
| 231:234 | | | | | | |
| breast normal GW98-621 | 23656 | 34.18 | 32.83 | 65.66 | breast normal | |
| breast tumor GW98-620 | 23655 | 35.32 | 16.75 | 33.50 | breast tumor | −1.96 |
| brain normal BB99-542 | 25507 | 34.01 | 36.19 | 72.38 | brain normal | |
| brain normal BB99-406 | 25509 | 31.9 | 124.88 | 249.76 | brain normal | |
| brain normal BB99-904 | 25546 | 34.98 | 20.53 | 41.06 | brain normal | |
| brain stage 5 ALZ BB99- | 25502 | 33.23 | 57.19 | 114.38 | brain stage 5 ALZ | −1.06 |
| 874 | | | | | | |
| brain stage 5 ALZ BB99- | 25503 | 31.38 | 169.5 | 339.00 | brain stage 5 ALZ | 2.80 |
| 887 | | | | | | |
| brain stage 5 ALZ BB99- | 25504 | 31.91 | 124.17 | 248.34 | brain stage 5 ALZ | 2.05 |
| 862 | | | | | | |
| brain stage 5 ALZ BB99- | 25542 | 31.33 | 174.15 | 348.30 | brain stage 5 ALZ | 2.88 |
| 927 | | | | | | |
| CT lung | normal | 40 | 0 | 0.00 | CT lung Nml | |
| lung 26 | normal | 35.19 | 18.16 | | lung 26 Nml | |
| lung 27 | normal | 40 | 0 | 0.00 | lung 27 Nml | |
| lung 24 | COPD | 39.56 | 1.4 | 1.40 | lung 24 COPD | 1.40 |
| lung 28 | COPD | 33.94 | 37.72 | 37.72 | lung 28 COPD | 37.72 |
| lung 23 | COPD | 35.4 | 16.05 | 16.05 | lung 23 COPD | 16.05 |
| lung 25 | normal | 40 | 0 | 0.00 | lung 25 Nml | |
| asthmatic lung ODO3112 | 29321 | 40 | 0 | 0.00 | asthmatic lung | 0.00 |
| asthmatic lung ODO3433 | 29323 | 40 | 0 | 0.00 | asthmatic lung | 0.00 |
| asthmatic lung ODO3397 | 29322 | 33.95 | 37.43 | 74.86 | asthmatic lung | 74.86 |
| asthmatic lung ODO4928 | 29325 | 36.06 | 10.86 | 21.72 | asthmatic lung | 21.72 |
| endo cells | control | 40 | 0 | 0.00 | endo cells | |
| endo VEGF | | 40 | 0 | 0.00 | endo VEGF | 0.00 |
| endo bFGF | | 40 | 0 | 0.00 | endo bFGF | 0.00 |
| heart Clontech | normal | 40 | 0 | 0.00 | heart | |
| heart (T-1) ischemic | 29417 | 34.3 | 30.58 | 61.16 | heart (T-1) | 61.16 |
| | | | | | ischemic | |
| heart (T-14) non- | 29422 | 35.14 | 18.72 | 37.44 | heart (T-14) non- | 37.44 |
| obstructive DCM | | | | | obstructive DCM | |
| heart (T-3399) DCM | 29426 | 35.05 | 19.72 | 39.44 | heart (T-3399) | 39.44 |
| | | | | | DCM | |
| adenoid GW99-269 | 26162 | 34.6 | 25.67 | 51.34 | adenoid | |
| tonsil GW98-280 | 22582 | 40 | 0 | 0.00 | tonsil | |
| T cells PC00314 | 28453 | 32 | 117.8 | 235.60 | T cells | |
| PBMNC | | 34.93 | 21.13 | 21.13 | PBMNC | |
| monocyte | | 36.87 | 6.76 | 13.52 | monocyte | |
| B cells PC00665 | 28455 | 33.17 | 59.12 | 118.24 | B cells | |
| dendritic cells | 28441 | 36.89 | 6.68 | 13.36 | dendritic cells | |
| neutrophils | 28440 | 32.49 | 88.11 | 88.11 | neutrophils | |
| eosinophils | 28446 | 40 | 0 | 0.00 | eosinophils | |
| BM unstim | | 36.4 | 8.92 | 8.92 | BM unstim | |
| BM stim | treated | 40 | 0 | 0.00 | BM stim | −8.92 |
| osteo dif | treated | 40 | 0 | 0.00 | osteo dif | −10.93 |
| osteo undif | | 36.05 | 10.93 | 10.93 | osteo undif | |
| chondrocytes | | 33.42 | 51.21 | 128.03 | chondrocytes | |
| OA Synovium IP12/01 | 29462 | 32.18 | 106.04 | 106.04 | OA Synovium | |
| OA Synovium NP10/01 | 29461 | 33.41 | 51.37 | 102.74 | OA Synovium | |
| OA Synovium NP57/00 | 28464 | 33.56 | 47.16 | 94.32 | OA Synovium | |
| RA Synovium | 28466 | 36.28 | 9.55 | 19.10 | RA Synovium | |
| NP03/01 | | | | | | |
| RA Synovium | 28467 | 33.3 | 54.77 | 109.54 | RA Synovium | |
| NP71/00 | | | | | | |
| RA Synovium | 28475 | 34.05 | 35.3 | 70.60 | RA Synovium | |
| NP45/00 | | | | | | |
| OA bone (biobank) | 29217 | 35.46 | 15.46 | 15.46 | OA bone | |
| | | | | | (biobank) | |
| OA bone Sample 1 | J. Emory | 35.93 | 11.75 | 23.50 | OA bone | |
| OA bone Sample 2 | J. Emory | 35.91 | 11.89 | 23.78 | OA bone | |
| Cartilage (pool) | Normal | 33.66 | 44.36 | 88.72 | Nml Cartilage | |
| | | | | | (pool) | |
| Cartilage (pool) | OA | 37.18 | 5.64 | 11.28 | OA Cartilage | −7.87 |
| | | | | | (pool) | |
| PBL unifected | 28441 | 35.46 | 15.44 | 30.88 | PBL unifected | |
| PBL HIV IIIB | 28442 | 40 | 0 | 0.00 | PBL HIV IIIB | −30.88 |
| MRC5 uninfected (100%) | 29158 | 40 | 0 | 0.00 | MRC5 uninfected | |
| | | | | | (100%) | |
| MRC5 HSV strain F | 29178 | 31.45 | 162.22 | 324.44 | MRC5 HSV | 324.44 |
| | | | | | strain F | |
| W12 cells | 29179 | 34.32 | 30.17 | 60.34 | W12 cells | 60.34 |
| Keratinocytes | 29180 | 40 | 0 | 0.00 | Keratinocytes | |
| B-actin control | | 28.82 | 758.28 | | | |
| genomic | | 28.02 | 1210.45 | | | |
| 1.00E+05 | | 20.55 | 100000 | | | |
| 1.00E+05 | | 20.69 | 100000 | | | |
| 1.00E+04 | | 24.32 | 10000 | | | |
| 1.00E+04 | | 24.25 | 10000 | | | |
| 1.00E+03 | | 28.04 | 1000 | | | |
| 1.00E+03 | | 27.94 | 1000 | | | |
| 1.00E+02 | | 33.09 | 100 | | | |
| 1.00E+02 | | 32.73 | 100 | | | |
| 1.00E+01 | | 36.83 | 10 | | | |
| 1.00E+01 | | 35.06 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name SBGKIN113

| | Fold Change in Disease |
|---|---|
| | Population Relative to |
| Disease tissues | Normal |
| colon tumor | −8.80 |
| colon tumor | 2.42 |
| colon tumor | 12.44 |
| colon tumor | −1.79 |
| lung tumor | −1.23 |
| lung tumor | 1.99 |
| lung tumor | 3.79 |
| lung tumor | −5.23 |
| breast tumor | 1.02 |
| breast tumor | 2.65 |
| breast tumor | −1.69 |
| breast tumor | −1.96 |
| brain stage 5 ALZ | −1.06 |
| brain stage 5 ALZ | 2.80 |
| brain stage 5 ALZ | 2.05 |
| brain stage 5 ALZ | 2.88 |
| lung 24 COPD | 1.40 |
| lung 28 COPD | 37.72 |
| lung 23 COPD | 16.05 |
| asthmatic lung | 0.00 |
| asthmatic lung | 0.00 |
| asthmatic lung | 74.86 |
| asthmatic lung | 21.72 |
| endo VEGF | 0.00 |
| endo bFGF | 0.00 |
| heart (T-1) ischemic | 61.16 |
| heart (T-14) non-obstructive DCM | 37.44 |
| heart (T-3399) DCM | 39.44 |
| BM stim | −8.92 |
| osteo dif | −10.93 |
| OA Cartilage (pool) | −7.87 |
| PBL HIV IIIB | −30.88 |
| MRC5 HSV strain F | 324.44 |
| W12 cells | 60.34 |

Gene Name gsk305961GDNa

Moderate overall expression. The highest normal expression is seen in the whole brain, fetal liver, and thymus. This gene is expressed fairly ubiquitously in the disease samples with the highest expression seen in the normal and disease brain samples. Significant upregulation in 1 of 4 colon tumor samples is sufficient to implicate this gene in colon cancer. Upregulation in 4 of 4 asthmatic lung samples with corroborating high expression in the neutrophils indicates a possible involvement in asthma. Upregulation in 3 of 3 disease heart samples implies an involvement in cardiovascular diseases such as ischemic, non-obstructive, and obstructive DCM. Upregulated in the stimulated bone marrow. Downregulation in the HIV-infected PBLs suggests that this gene may be a host factor in HIV. Moderate expression in the RA and OA synovium samples, the OA bone samples, and the chondrocytes with corroborating high expression in T cells, B cells, and neutrophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| gsk305961GDNa | 1 and 2) | 1) | 2) | Copies | (ng) | (ng) | RNA |
| Subcutaneous | 32.72, 32.61 | 51.9 | 55.65 | 53.78 | 3.06 | 16.34 | 878.68 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland | 36.16, 40 | 5.89 | 0.22 | 3.06 | 0.61 | 81.97 | 250.41 |
| Clontech | | | | | | | |
| Whole Brain Clontech | 26.92, 26.79 | 2015.63 | 2193.88 | 2104.76 | 7.24 | 6.91 | 14535.60 |
| Fetal Brain Clontech | 40, 36.32 | 0 | 5.32 | 2.66 | 0.48 | 103.95 | 276.51 |
| Cerebellum Clontech | 32.18, 34.16 | 72.63 | 20.87 | 46.75 | 2.17 | 23.04 | 1077.19 |
| Cervix | 33.63, 35.29 | 29.2 | 10.2 | 19.70 | 2.42 | 20.66 | 407.02 |
| Colon | 33.52, 33.7 | 31.18 | 27.97 | 29.58 | 2.71 | 18.45 | 545.66 |
| Endometrium | 40, 34.82 | 0 | 13.78 | 6.89 | 0.73 | 68.21 | 469.99 |
| Esophagus | 35.08, 36.31 | 11.69 | 5.37 | 8.53 | 1.37 | 36.50 | 311.31 |
| Heart Clontech | 34.03, 40 | 22.66 | 0 | 11.33 | 1.32 | 37.88 | 429.17 |
| Hypothalamus | 40, 40 | 0 | 0 | 0.00 | 0.32 | 155.28 | 0.00 |
| Ileum | 33.89, 34.69 | 24.7 | 14.95 | 19.83 | 2.58 | 19.38 | 384.21 |
| Jejunum | 30.83, 31.14 | 171.04 | 140.83 | 155.94 | 6.60 | 7.58 | 1181.33 |
| Kidney | 33.44, 35.58 | 32.85 | 8.48 | 20.67 | 2.12 | 23.58 | 487.38 |
| Liver | 34.7, 35.29 | 14.84 | 10.23 | 12.54 | 1.50 | 33.33 | 417.83 |
| Fetal Liver Clontech | 28.72, 28.57 | 648.19 | 714.17 | 681.18 | 10.40 | 4.81 | 3274.90 |
| Lung | 34.4, 32.94 | 17.88 | 45.19 | 31.54 | 2.57 | 19.46 | 613.52 |
| Mammary Gland | 32.19, 31.45 | 72.29 | 115.73 | 94.01 | 13.00 | 3.85 | 361.58 |
| Clontech | | | | | | | |
| Myometrium | 33.49, 32.51 | 31.81 | 59.28 | 45.55 | 2.34 | 21.37 | 973.18 |
| Omentum | 33.2, 33.62 | 38.15 | 29.28 | 33.72 | 3.94 | 12.69 | 427.86 |
| Ovary | 33, 32.48 | 43.27 | 60.2 | 51.74 | 4.34 | 11.52 | 596.03 |
| Pancreas | 35.27, 35.07 | 10.34 | 11.76 | 11.05 | 0.81 | 61.80 | 682.94 |
| Head of Pancreas | 40, 40 | 0.29 | 0 | 0.15 | 1.57 | 31.85 | 4.62 |
| Parotid Gland | 33.39, 33.02 | 33.89 | 42.95 | 38.42 | 5.48 | 9.12 | 350.55 |
| Placenta Clontech | 33.89, 34.6 | 24.71 | 15.84 | 20.28 | 5.26 | 9.51 | 192.73 |
| Prostate | 36.23, 34.18 | 5.66 | 20.59 | 13.13 | 3.00 | 16.67 | 218.75 |
| Rectum | 34.28, 33.61 | 19.36 | 29.53 | 24.45 | 1.23 | 40.65 | 993.70 |
| Salivary Gland | 33.8, 34.21 | 26.16 | 20.22 | 23.19 | 7.31 | 6.84 | 158.62 |
| Clontech | | | | | | | |
| Skeletal Muscle | 40, 40 | 0 | 0 | 0.00 | 1.26 | 39.68 | 0.00 |
| Clontech | | | | | | | |
| Skin | 36.07, 40 | 6.23 | 0 | 3.12 | 1.21 | 41.32 | 128.72 |
| Small Intestine | 40, 40 | 0 | 0 | 0.00 | 0.98 | 51.07 | 0.00 |
| Clontech | | | | | | | |
| Spleen | 32.2, 32.12 | 71.71 | 75.66 | 73.69 | 4.92 | 10.16 | 748.83 |
| Stomach | 33.35, 32.97 | 34.86 | 44.16 | 39.51 | 2.73 | 18.32 | 723.63 |
| Testis Clontech | 40, 40 | 0 | 0.32 | 0.16 | 0.57 | 87.87 | 14.06 |
| Thymus Clontech | 29.86, 29.38 | 316.1 | 426.56 | 371.33 | 9.89 | 5.06 | 1877.30 |
| Thyroid | 34.89, 34.13 | 13.15 | 21.19 | 17.17 | 2.77 | 18.05 | 309.93 |
| Trachea Clontech | 35.19, 35.4 | 10.86 | 9.54 | 10.20 | 9.71 | 5.15 | 52.52 |
| Urinary Bladder | 32.64, 32.29 | 54.45 | 68.08 | 61.27 | 5.47 | 9.14 | 560.01 |
| Uterus | 31.21, 31.42 | 134.07 | 117.35 | 125.71 | 5.34 | 9.36 | 1177.06 |
| genomic | 27.62 | 1298.05 | | | | | |
| b-actin | 28.32 | 835.8 | | | | | |
| 1.00E+05 | 20.94 | 100000 | | | | | |
| 1.00E+05 | 21.05 | 100000 | | | | | |
| 1.00E+04 | 24.22 | 10000 | | | | | |
| 1.00E+04 | 24.21 | 10000 | | | | | |
| 1.00E+03 | 27.57 | 1000 | | | | | |
| 1.00E+03 | 27.69 | 1000 | | | | | |
| 1.00E+02 | 31.86 | 100 | | | | | |
| 1.00E+02 | 32.15 | 100 | | | | | |
| 1.00E+01 | 40 | 0 | | | | | |
| 1.00E+01 | 40 | 0 | | | | | |
| 1.00E+00 | 40 | 0 | | | | | |
| 1.00E+00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| gsk305961GDNa | identifier) | Ct | copies | RNA | Sample | Population |
| colon normal GW98-167 | 21941 | 28.26 | 802.94 | 1605.88 | colon normal | |
| colon tumor GW98-166 | 21940 | 28.83 | 567.11 | 1134.22 | colon tumor | −1.42 |
| colon normal GW98-178 | 22080 | 31.06 | 145.98 | 291.96 | colon normal | |
| colon tumor GW98-177 | 22060 | 27.16 | 1571.35 | 3142.70 | colon tumor | 10.76 |
| colon normal GW98-561 | 23514 | 29.52 | 373.73 | 747.46 | colon normal | |
| colon tumor GW98-560 | 23513 | 30.07 | 267.13 | 534.26 | colon tumor | −1.40 |
| colon normal GW98-894 | 24691 | 28.43 | 725.8 | 1451.60 | colon normal | |
| colon tumor GW98-893 | 24690 | 30.03 | 273.24 | 546.48 | colon tumor | −2.66 |
| lung normal GW98-3 | 20742 | 28.6 | 653.04 | 1306.08 | lung normal | |
| lung tumor GW98-2 | 20741 | 31.32 | 124.74 | 249.48 | lung tumor | −5.24 |
| lung normal GW97-179 | 20677 | 28.48 | 700.84 | 1401.68 | lung normal | |
| lung tumor GW97-178 | 20676 | 28.59 | 657.27 | 1314.54 | lung tumor | −1.07 |
| lung normal GW98-165 | 21922 | 28.28 | 792.39 | 1584.78 | lung normal | |
| lung tumor GW98-164 | 21921 | 27.54 | 1243.98 | 2487.96 | lung tumor | 1.57 |
| lung normal GW98-282 | 22584 | 28.91 | 541.48 | 1082.96 | lung normal | |
| lung tumor GW98-281 | 22583 | 29.46 | 385.64 | 771.28 | lung tumor | −1.40 |
| breast normal GW00-392 | 28750 | 29.65 | 345.37 | 345.37 | breast normal | |
| breast tumor GW00-391 | 28746 | 29.18 | 459.56 | 919.12 | breast tumor | 2.66 |
| breast normal GW00-413 | 28798 | 29.27 | 434.11 | 434.11 | breast normal | |
| breast tumor GW00-412 | 28797 | 29.94 | 288.46 | 576.92 | breast tumor | 1.33 |
| breast normal GW00- | 27592-95 | 30.22 | 242.78 | 242.78 | breast normal | |
| 235:238 | | | | | | |
| breast tumor GW00- | 27588-91 | 30.32 | 228.69 | 228.69 | breast tumor | −1.06 |
| 231:234 | | | | | | |
| breast normal GW98-621 | 23656 | 27.25 | 1489.15 | 2978.30 | breast normal | |
| breast tumor GW98-620 | 23655 | 27.45 | 1313.03 | 2626.06 | breast tumor | −1.13 |
| brain normal BB99-542 | 25507 | 27.92 | 988.42 | 1976.84 | brain normal | |
| brain normal BB99-406 | 25509 | 28.21 | 826.32 | 1652.64 | brain normal | |
| brain normal BB99-904 | 25546 | 28.43 | 723.46 | 1446.92 | brain normal | |
| brain stage 5 ALZ BB99- | 25502 | 29.67 | 341.17 | 682.34 | brain stage 5 ALZ | −2.48 |
| 874 | | | | | | |
| brain stage 5 ALZ BB99- | 25503 | 27.83 | 1043.54 | 2087.08 | brain stage 5 ALZ | 1.23 |
| 887 | | | | | | |
| brain stage 5 ALZ BB99- | 25504 | 28.59 | 657.15 | 1314.30 | brain stage 5 ALZ | −1.29 |
| 862 | | | | | | |
| brain stage 5 ALZ BB99- | 25542 | 27.57 | 1222.93 | 2445.86 | brain stage 5 ALZ | 1.45 |
| 927 | | | | | | |
| CT lung | normal | 33.3 | 37.35 | 74.70 | CT lung Nml | |
| lung 26 | normal | 32.08 | 78.26 | | lung 26 Nml | |
| lung 27 | normal | 32.45 | 62.64 | 62.64 | lung 27 Nml | |
| lung 24 | COPD | 33.08 | 42.63 | 42.63 | lung 24 COPD | −1.87 |
| lung 28 | COPD | 31.63 | 102.87 | 102.87 | lung 28 COPD | 1.29 |
| lung 23 | COPD | 32.02 | 81.24 | 81.24 | lung 23 COPD | 1.02 |
| lung 25 | normal | 31.64 | 102.34 | 102.34 | lung 25 Nml | |
| asthmatic lung ODO3112 | 29321 | 30.37 | 222.52 | 222.52 | asthmatic lung | 2.79 |
| asthmatic lung ODO3433 | 29323 | 29.48 | 381.78 | 763.56 | asthmatic lung | 9.56 |
| asthmatic lung ODO3397 | 29322 | 27.42 | 1336.97 | 2673.94 | asthmatic lung | 33.47 |
| asthmatic lung ODO4928 | 29325 | 28.55 | 674.55 | 1349.10 | asthmatic lung | 16.89 |
| endo cells | control | 32.1 | 77.45 | 77.45 | endo cells | |
| endo VEGF | | 31.93 | 86.01 | 86.01 | endo VEGF | 1.11 |
| endo bFGF | | 31.63 | 102.85 | 102.85 | endo bFGF | 1.33 |
| heart Clontech | normal | 35.45 | 10.07 | 20.14 | heart | |
| heart (T-1) ischemic | 29417 | 28.37 | 750.43 | 1500.86 | heart (T-1) | 74.52 |
| | | | | | ischemic | |
| heart (T-14) non- | 29422 | 29.5 | 376.98 | 753.96 | heart (T-14) non- | 37.44 |
| obstructive DCM | | | | | obstructive DCM | |
| heart (T-3399) DCM | 29426 | 28.63 | 641.88 | 1283.76 | heart (T-3399) | 63.74 |
| | | | | | DCM | |
| adenoid GW99-269 | 26162 | 29.72 | 330.41 | 660.82 | adenoid | |
| tonsil GW98-280 | 22582 | 28.03 | 922.22 | 1844.44 | tonsil | |
| T cells PC00314 | 28453 | 29.66 | 342.13 | 684.26 | T cells | |
| PBMNC | | 33.44 | 34.3 | 34.30 | PBMNC | |
| monocyte | | 34.41 | 18.95 | 37.90 | monocyte | |
| B cells PC00665 | 28455 | 28.69 | 617.46 | 1234.92 | B cells | |
| dendritic cells | 28441 | 30.55 | 198.74 | 397.48 | dendritic cells | |
| neutrophils | 28440 | 28.89 | 547.36 | 547.36 | neutrophils | |
| eosinophils | 28446 | 32.86 | 48.74 | 97.48 | eosinophils | |
| BM unstim | | 34.4 | 19.08 | 19.08 | BM unstim | |
| BM stim | treated | 31.71 | 98.33 | 98.33 | BM stim | 5.15 |
| osteo dif | treated | 32.17 | 74.22 | 74.22 | osteo dif | 1.75 |
| osteo undif | | 33.09 | 42.38 | 42.38 | osteo undif | |
| chondrocytes | | 29.45 | 388.3 | 970.75 | chondrocytes | |
| OA Synovium IP12/01 | 29462 | 28.49 | 696.93 | 696.93 | OA Synovium | |
| OA Synovium NP10/01 | 29461 | 30.09 | 263.3 | 526.60 | OA Synovium | |
| OA Synovium NP57/00 | 28464 | 29.15 | 466.7 | 933.40 | OA Synovium | |
| RA Synovium | 28466 | 29.06 | 493.54 | 987.08 | RA Synovium | |
| NP03/01 | | | | | | |
| RA Synovium | 28467 | 28.7 | 615.4 | 1230.80 | RA Synovium | |
| NP71/00 | | | | | | |
| RA Synovium | 28475 | 29.13 | 473.65 | 947.30 | RA Synovium | |
| NP45/00 | | | | | | |
| OA bone (biobank) | 29217 | 30 | 278.56 | 278.56 | OA bone (biobank) | |
| OA bone Sample 1 | J. Emory | 28.74 | 600.15 | 1200.30 | OA bone | |
| OA bone Sample 2 | J. Emory | 29.24 | 442.37 | 884.74 | OA bone | |
| Cartilage (pool) | Normal | 28.1 | 887.94 | 1775.88 | Nml Cartilage | |
| | | | | | (pool) | |
| Cartilage (pool) | OA | 30.04 | 271.28 | 542.56 | OA Cartilage (pool) | −3.27 |
| PBL unifected | 28441 | 27.65 | 1165.13 | 2330.26 | PBL unifected | |
| PBL HIV IIIB | 28442 | 29.8 | 313.52 | 627.04 | PBL HIV IIIB | −3.72 |
| MRC5 uninfected | 29158 | 29 | 510.52 | 1021.04 | MRC5 uninfected | |
| (100%) | | | | | (100%) | |
| MRC5 HSV strain F | 29178 | 29.46 | 387.61 | 775.22 | MRC5 HSV strain F | −1.32 |
| W12 cells | 29179 | 27.76 | 1090.41 | 2180.82 | W12 cells | 1.28 |
| Keratinocytes | 29180 | 28.16 | 853.46 | 1706.92 | Keratinocytes | |
| B-actin control | | 28.21 | 830.7 | | | |
| genomic | | 27.27 | 1465.59 | | | |
| 1.00E+05 | | 20.48 | 100000 | | | |
| 1.00E+05 | | 20.43 | 100000 | | | |
| 1.00E+04 | | 23.9 | 10000 | | | |
| 1.00E+04 | | 24.02 | 10000 | | | |
| 1.00E+03 | | 27.55 | 1000 | | | |
| 1.00E+03 | | 27.46 | 1000 | | | |
| 1.00E+02 | | 32.83 | 100 | | | |
| 1.00E+02 | | 32.19 | 100 | | | |
| 1.00E+01 | | 34.68 | 10 | | | |
| 1.00E+01 | | 40 | 0 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name gsk305961GDNa

| | Fold Change in Disease |
|---|---|
| | Population Relative to |
| Disease tissues | Normal |
| colon tumor | −1.42 |
| colon tumor | 10.76 |
| colon tumor | −1.40 |
| colon tumor | −2.66 |
| lung tumor | −5.24 |
| lung tumor | −1.07 |
| lung tumor | 1.57 |
| lung tumor | −1.40 |
| breast tumor | 2.66 |
| breast tumor | 1.33 |
| breast tumor | −1.06 |
| breast tumor | −1.13 |
| brain stage 5 ALZ | −2.48 |
| brain stage 5 ALZ | 1.23 |
| brain stage 5 ALZ | −1.29 |
| brain stage 5 ALZ | 1.45 |
| lung 24 COPD | −1.87 |
| lung 28 COPD | 1.29 |
| lung 23 COPD | 1.02 |
| asthmatic lung | 2.79 |
| asthmatic lung | 9.56 |
| asthmatic lung | 33.47 |
| asthmatic lung | 16.89 |
| endo VEGF | 1.11 |
| endo bFGF | 1.33 |
| heart (T-1) ischemic | 74.52 |
| heart (T-14) non-obstructive DCM | 37.44 |
| heart (T-3399) DCM | 63.74 |
| BM stim | 5.15 |
| osteo dif | 1.75 |
| OA Cartilage (pool) | −3.27 |
| PBL HIV IIIB | −3.72 |
| MRC5 HSV strain F | −1.32 |
| W12 cells | 1.28 |

Gene Name gsk2402719_{—}2395124BIG2

Moderate overall expression. The highest normal expression is seen in the whole brain, endometrium, fetal liver, rectum, and thymus. The highest disease expression is seen in the normal and disease brain samples. Downregulation in 1 of 4 colon tumor samples and 1 of 4 lung tumor samples implicates this gene in cancers of the colon and lung. Upregulation in 3 of 3 disease heart samples implies an involvement in cardiovascular diseases such as non-obstructive and obstructive DCM and ischemia. Upregulation in the H-infected PBLs suggests that this gene may be a host factor in HIV.

| | | | | | | | copies of |
|---|---|---|---|---|---|---|---|
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| gsk2402719_2395124BIG2 | 1 and 2) | 1) | 2) | Copies | (ng) | (ng) | RNA |
| Subcutaneous | 35.28, 34.85 | 6.1 | 8.03 | 7.07 | 3.06 | 16.34 | 115.44 |
| Adipocytes Zenbio | | | | | | | |
| Subcutaneous Adipose | 40, 40 | 1.25 | 0.96 | 1.11 | 0.96 | 52.36 | 57.85 |
| Zenbio | | | | | | | |
| Adrenal Gland Clontech | 40, 40 | 0 | 0 | 0.00 | 0.61 | 81.97 | 0.00 |
| Whole Brain Clontech | 23.66, 23.25 | 10169.71 | 13215.56 | 11692.64 | 7.24 | 6.91 | 80750.24 |
| Fetal Brain Clontech | 36.72, 40 | 2.43 | 1 | 1.72 | 0.48 | 103.95 | 178.27 |
| Cerebellum Clontech | 34.22, 33.46 | 11.99 | 19.44 | 15.72 | 2.17 | 23.04 | 362.10 |
| Cervix | 33.97, 32.73 | 14.1 | 31.04 | 22.57 | 2.42 | 20.66 | 466.32 |
| Colon | 33.7, 33.26 | 16.72 | 22.21 | 19.47 | 2.71 | 18.45 | 359.13 |
| Endometrium | 34.88, 33.41 | 7.88 | 20.12 | 14.00 | 0.73 | 68.21 | 954.98 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 36.05, 36.11 | 3.72 | 3.59 | 3.66 | 2.58 | 19.38 | 70.83 |
| Jejunum | 33.01, 33.17 | 25.92 | 23.51 | 24.72 | 6.60 | 7.58 | 187.23 |
| Kidney | 36.85, 35.29 | 2.24 | 6.06 | 4.15 | 2.12 | 23.58 | 97.88 |
| Liver | 34.92, 35.91 | 7.68 | 4.07 | 5.88 | 1.50 | 33.33 | 195.83 |
| Fetal Liver Clontech | 29.86, 29.34 | 194.37 | 270.22 | 232.30 | 10.40 | 4.81 | 1116.80 |
| Lung | 40, 40 | 0 | 0 | 0.00 | 2.57 | 19.46 | 0.00 |
| Mammary Gland | 31.65, 30.95 | 61.84 | 96.67 | 79.26 | 13.00 | 3.85 | 304.83 |
| Clontech | | | | | | | |
| Myometrium | 34.98, 34.06 | 7.38 | 13.25 | 10.32 | 2.34 | 21.37 | 220.41 |
| Omentum | 40, 34.96 | 0 | 7.45 | 3.73 | 3.94 | 12.69 | 47.27 |
| Ovary | 33.61, 35.48 | 17.76 | 5.36 | 11.56 | 4.34 | 11.52 | 133.18 |
| Pancreas | 40, 40 | 0 | 0 | 0.00 | 0.81 | 61.80 | 0.00 |
| Head of Pancreas | 35.75, 40 | 4.53 | 0.96 | 2.75 | 1.57 | 31.85 | 87.42 |
| Parotid Gland | 34.71, 36.01 | 8.77 | 3.84 | 6.31 | 5.48 | 9.12 | 57.53 |
| Placenta Clontech | 33.98, 33.9 | 13.94 | 14.7 | 14.32 | 5.26 | 9.51 | 136.12 |
| Prostate | 40, 40 | 1.26 | 0 | 0.63 | 3.00 | 16.67 | 10.50 |
| Rectum | 34.54, 33.29 | 9.8 | 21.73 | 15.77 | 1.23 | 40.65 | 640.85 |
| Salivary Gland Clontech | 35.9, 36 | 4.1 | 3.85 | 3.98 | 7.31 | 6.84 | 27.19 |
| Skeletal Muscle | 40, 40 | 0 | 0 | 0.00 | 1.26 | 39.68 | 0.00 |
| Clontech | | | | | | | |
| Skin | 40, 40 | 1.42 | 1.24 | 1.33 | 1.21 | 41.32 | 54.96 |
| Small Intestine Clontech | 39.43, 35.47 | 0.43 | 5.4 | 2.92 | 0.98 | 51.07 | 148.88 |
| Spleen | 35.92, 40 | 4.06 | 0 | 2.03 | 4.92 | 10.16 | 20.63 |
| Stomach | 40, 33.97 | 0 | 14.08 | 7.04 | 2.73 | 18.32 | 128.94 |
| Testis Clontech | 35.01, 40 | 7.26 | 0 | 3.63 | 0.57 | 87.87 | 318.98 |
| Thymus Clontech | 29, 28.19 | 335.07 | 563.44 | 449.26 | 9.89 | 5.06 | 2271.26 |
| Thyroid | 34.82, 33.73 | 8.2 | 16.41 | 12.31 | 2.77 | 18.05 | 222.11 |
| Trachea Clontech | 33.57, 33.17 | 18.13 | 23.47 | 20.80 | 9.71 | 5.15 | 107.11 |
| Urinary Bladder | 32.07, 32.42 | 47.27 | 37.8 | 42.54 | 5.47 | 9.14 | 388.80 |
| Uterus | 32.44, 31.61 | 37.44 | 63.44 | 50.44 | 5.34 | 9.36 | 472.28 |
| genomic | 26.63 | 1526.9 | | | | | |
| b-actin | 28.11 | 594.87 | | | | | |
| 1.00E+05 | 19.85 | 100000 | | | | | |
| 1.00E+05 | 19.99 | 100000 | | | | | |
| 1.00E+04 | 23.47 | 10000 | | | | | |
| 1.00E+04 | 23.62 | 10000 | | | | | |
| 1.00E+03 | 27.23 | 1000 | | | | | |
| 1.00E+03 | 27.29 | 1000 | | | | | |
| 1.00E+02 | 32.1 | 100 | | | | | |
| 1.00E+02 | 31.16 | 100 | | | | | |
| 1.00E+01 | 40 | 10 | | | | | |
| 1.00E+01 | 40 | 10 | | | | | |
| 1.00E+00 | 40 | 0 | | | | | |
| 1.00E+00 | 37.31 | 1 | | | | | |
| NTC | 40 | −1 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| gsk2402719_2395124BIG2 | identifier) | Ct | copies | RNA | Sample | Population |
| colon normal GW98-167 | 21941 | 26.46 | 2706.74 | 5413.48 | colon normal | |
| colon tumor GW98-166 | 21940 | 26.85 | 2226.83 | 4453.66 | colon tumor | −1.22 |
| colon normal GW98-178 | 22080 | 31 | 271.46 | 542.92 | colon normal | |
| colon tumor GW98-177 | 22060 | 30.76 | 306.61 | 613.22 | colon tumor | 1.13 |
| colon normal GW98-561 | 23514 | 28 | 1240.55 | 2481.10 | colon normal | |
| colon tumor GW98-560 | 23513 | 29 | 749.77 | 1499.54 | colon tumor | −1.65 |
| colon normal GW98-894 | 24691 | 26.66 | 2452.21 | 4904.42 | colon normal | |
| colon tumor GW98-893 | 24690 | 31.94 | 169.13 | 338.26 | colon tumor | −14.50 |
| lung normal GW98-3 | 20742 | 28.13 | 1163 | 2326.00 | lung normal | |
| lung tumor GW98-2 | 20741 | 36.81 | 14.4 | 28.80 | lung tumor | −80.76 |
| lung normal GW97-179 | 20677 | 28.45 | 991.35 | 1982.70 | lung normal | |
| lung tumor GW97-178 | 20676 | 27.75 | 1410.69 | 2821.38 | lung tumor | 1 .42 |
| lung normal GW98-165 | 21922 | 27.45 | 1643.91 | 3287.82 | lung normal | |
| lung tumor GW98-164 | 21921 | 29.31 | 638.73 | 1277.46 | lung tumor | −2.57 |
| lung normal GW98-282 | 22584 | 30.26 | 396.16 | 792.32 | lung normal | |
| lung tumor GW98-281 | 22583 | 29.08 | 719.66 | 1439.32 | lung tumor | 1.82 |
| breast normal GW00-392 | 28750 | 29.06 | 727.15 | 727.15 | breast normal | |
| breast tumor GW00-391 | 28746 | 29.29 | 645.27 | 1290.54 | breast tumor | 1.77 |
| breast normal GW00-413 | 28798 | 30.26 | 395.16 | 395.16 | breast normal | |
| breast tumor GW00-412 | 28797 | 28.42 | 1002.1 | 2004.20 | breast tumor | 5.07 |
| breast normal GW00- | 27592-95 | 30.32 | 383.84 | 383.84 | breast normal | |
| 235:238 | | | | | | |
| breast tumor GW00- | 27588-91 | 28.51 | 961.05 | 961.05 | breast tumor | 2.50 |
| 231:234 | | | | | | |
| breast normal GW98-621 | 23656 | 29.01 | 743.09 | 1486.18 | breast normal | |
| breast tumor GW98-620 | 23655 | 30.46 | 357.2 | 714.40 | breast tumor | −2.08 |
| brain normal BB99-542 | 25507 | 25.12 | 5327.67 | 10655.34 | brain normal | |
| brain normal BB99-406 | 25509 | 27.83 | 1351.64 | 2703.28 | brain normal | |
| brain normal BB99-904 | 25546 | 25.75 | 3870.14 | 7740.28 | brain normal | |
| brain stage 5 ALZ BB99- | 25502 | 29.43 | 601.57 | 1203.14 | brain stage 5 ALZ | −5.85 |
| 874 | | | | | | |
| brain stage 5 ALZ BB99- | 25503 | 26.29 | 2947.47 | 5894.94 | brain stage 5 ALZ | −1.19 |
| 887 | | | | | | |
| brain stage 5 ALZ BB99- | 25504 | 26.45 | 2727.55 | 5455.10 | brain stage 5 ALZ | −1.29 |
| 862 | | | | | | |
| brain stage 5 ALZ BB99- | 25542 | 26.39 | 2803.89 | 5607.78 | brain stage 5 ALZ | −1.25 |
| 927 | | | | | | |
| CT lung | normal | 29.27 | 654.09 | 1308.18 | CT lung Nml | |
| lung 26 | normal | 38.1 | 7.47 | | lung 26 Nml | |
| lung 27 | normal | 35.28 | 31.21 | 31.21 | lung 27 Nml | |
| lung 24 | COPD | 34.34 | 50.08 | 50.08 | lung 24 COPD | −9.42 |
| lung 28 | COPD | 35.23 | 31.94 | 31.94 | lung 28 COPD | −14.77 |
| lung 23 | COPD | 35.24 | 31.78 | 31.78 | lung 23 COPD | −14.85 |
| lung 25 | normal | 33.51 | 76.24 | 76.24 | lung 25 Nml | |
| asthmatic lung ODO3112 | 29321 | 30.3 | 388.5 | 388.50 | asthmatic lung | −1.21 |
| asthmatic lung ODO3433 | 29323 | 29.02 | 742.53 | 1485.06 | asthmatic lung | 3.15 |
| asthmatic lung ODO3397 | 29322 | 27.36 | 1717.68 | 3435.36 | asthmatic lung | 7.28 |
| asthmatic lung ODO4928 | 29325 | 28.12 | 1167.33 | 2334.66 | asthmatic lung | 4.95 |
| endo cells | control | 40 | 0 | 0.00 | endo cells | |
| endo VEGF | | 40 | 0 | 0.00 | endo VEGF | 0.00 |
| endo bFGF | | 40 | 0 | 0.00 | endo bFGF | 0.00 |
| heart Clontech | normal | 32.49 | 128.22 | 256.44 | heart | |
| heart (T-1) ischemic | 29417 | 28.23 | 1104.84 | 2209.68 | heart (T-1) | 8.62 |
| | | | | | ischemic | |
| heart (T-14) non- | 29422 | 29.35 | 626.6 | 1253.20 | heart (T-14) non- | 4.89 |
| obstructive DCM | | | | | obstructive DCM | |
| heart (T-3399) DCM | 29426 | 28.69 | 876.16 | 1752.32 | heart (T-3399) | 6.83 |
| | | | | | DCM | |
| adenoid GW99-269 | 26162 | 29.3 | 641.83 | 1283.66 | adenoid | |
| tonsil GW98-280 | 22582 | 28.29 | 1074.11 | 2148.22 | tonsil | |
| T cells PC00314 | 28453 | 35.2 | 32.49 | 64.98 | T cells | |
| PBMNC | | 40 | 0 | 0.00 | PBMNC | |
| monocyte | | 40 | 0 | 0.00 | monocyte | |
| B cells PC00665 | 28455 | 40 | 2.73 | 5.46 | B cells | |
| dendritic cells | 28441 | 35.31 | 30.64 | 61.28 | dendritic cells | |
| neutrophils | 28440 | 32.41 | 133.12 | 133.12 | neutrophils | |
| eosinophils | 28446 | 37.05 | 12.74 | 25.48 | eosinophils | |
| BM unstim | | 40 | 0 | 0.00 | BM unstim | |
| BM stim | treated | 40 | 0 | 0.00 | BM stim | 0.00 |
| osteo dif | treated | 40 | 0 | 0.00 | osteo dif | 0.00 |
| osteo undif | | 40 | 0 | 0.00 | osteo undif | |
| chondrocytes | | 40 | 0 | 0.00 | chondrocytes | |
| OA Synovium IP12/01 | 29462 | 26.63 | 2484.1 | 2484.10 | OA Synovium | |
| OA Synovium NP10/01 | 29461 | 29.13 | 702.54 | 1405.08 | OA Synovium | |
| OA Synovium NP57/00 | 28464 | 26.22 | 3050.86 | 6101.72 | OA Synovium | |
| RA Synovium | 28466 | 31.63 | 197.53 | 395.06 | RA Synovium | |
| NP03/01 | | | | | | |
| RA Synovium | 28467 | 29.23 | 665.35 | 1330.70 | RA Synovium | |
| NP71/00 | | | | | | |
| RA Synovium | 28475 | 31.98 | 165.38 | 330.76 | RA Synovium | |
| NP45/00 | | | | | | |
| OA bone (biobank) | 29217 | 35.46 | 28.46 | 28.46 | OA bone | |
| | | | | | (biobank) | |
| OA bone Sample 1 | J. Emory | 31.87 | 175.21 | 350.42 | OA bone | |
| OA bone Sample 2 | J. Emory | 32.2 | 148.1 | 296.20 | OA bone | |
| Cartilage (pool) | Normal | 28.88 | 796.08 | 1592.16 | Nml Cartilage | |
| | | | | | (pool) | |
| Cartilage (pool) | OA | 30.26 | 396.09 | 792.18 | OA Cartilage | −2.01 |
| | | | | | (pool) | |
| PBL unifected | 28441 | 40 | 0 | 0.00 | PBL unifected | |
| PBL HIV IIIB | 28442 | 39.92 | 3.8 | 7.60 | PBL HIV IIIB | 7.60 |
| MRC5 uninfected (100%) | 29158 | 34.46 | 47.26 | 94.52 | MRC5 uninfected | |
| | | | | | (100%) | |
| MRC5 HSV strain F | 29178 | 34.7 | 41.87 | 83.74 | MRC5 HSV | −1.13 |
| | | | | | strain F | |
| W12 cells | 29179 | 40 | 3.66 | 7.32 | W12 cells | 1.02 |
| Keratinocytes | 29180 | 40 | 3.58 | 7.16 | Keratinocytes | |
| B-actin control | | 27.25 | 1815.19 | | | |
| genomic | | 27.3 | 1773.27 | | | |
| 1.00E+05 | | 20 | 100000 | | | |
| 1.00E+05 | | 20.27 | 100000 | | | |
| 1.00E+04 | | 23.63 | 10000 | | | |
| 1.00E+04 | | 23.66 | 10000 | | | |
| 1.00E+03 | | 27.18 | 1000 | | | |
| 1.00E+03 | | 27.37 | 1000 | | | |
| 1.00E+02 | | 33.46 | 100 | | | |
| 1.00E+02 | | 32.03 | 100 | | | |
| 1.00E+01 | | 39.15 | 10 | | | |
| 1.00E+01 | | 40 | 10 | | | |
| 1.00E+00 | | 40 | 0 | | | |
| 1.00E+00 | | 40 | 0 | | | |
| NTC | | 40 | 0 | | | |

*lung 26 Normal has been omitted due to multiple amplification failures from that sample

Gene Name gsk2402719_{—}2395124BIG2

| | Fold Change in Disease |
|---|---|
| | Population Relative to |
| Disease tissues | Normal |
| colon tumor | −1.22 |
| colon tumor | 1.13 |
| colon tumor | −1.65 |
| colon tumor | −14.50 |
| lung tumor | −80.76 |
| lung tumor | 1.42 |
| lung tumor | −2.57 |
| lung tumor | 1.82 |
| breast tumor | 1.77 |
| breast tumor | 5.07 |
| breast tumor | 2.50 |
| breast tumor | −2.08 |
| brain stage 5 ALZ | −5.85 |
| brain stage 5 ALZ | −1.19 |
| brain stage 5 ALZ | −1.29 |
| brain stage 5 ALZ | −1.25 |
| lung 24 COPD | −9.42 |
| lung 28 COPD | −14.77 |
| lung 23 COPD | −14.85 |
| asthmatic lung | −1.21 |
| asthmatic lung | 3.15 |
| asthmatic lung | 7.28 |
| asthmatic lung | 4.95 |
| endo VEGF | 0.00 |
| endo bFGF | 0.00 |
| heart (T-1) ischemic | 8.62 |
| heart (T-14) non-obstructive DCM | 4.89 |
| heart (T-3399) DCM | 6.83 |
| BM stim | 0.00 |
| osteo dif | 0.00 |
| OA Cartilage (pool) | −2.01 |
| PBL HIV IIIB | 7.60 |
| MRC5 HSV strain F | −1.13 |
| W12 cells | 1.02 |

Gene Name sbg102200MCTc

High to moderate overall expression. Highest normal expression in the whole brain, liver, fetal liver, and thymus. Highest disease expression in one of the colon normal/tumor pairs, one of the lung normal/tumor pairs, one of the asthmatic lung samples, the dendritic cells, and the uninfected and HIV-infected PBL cells. Upregulation in 2 of 4 breast tumor samples is sufficient to make a disease claim in cancer of the breast. Upregulation in 1 of 4 AD brain samples indicates a potential role in Alzheimer's disease. Downregulation in 3 of 3 COPD lung samples suggests involvement in chronic obstructive pulmonary disease. Upregulation in 1 of 4 asthmatic lung samples indicates a potential role for this gene in lung cancer. High expression in all of the immune cells. Also high to moderate expression in the OA and RA synovium samples, the OA bone samples, and in the chondrocytes suggests an involvement in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | Ct | copies | copies | Average | 18 S | 18 S | 50 ng |
| Sample | (sample | (sample | (sample | GOI | rRNA | rRNA | total |
| sbg102200MCTc | 1 and 2) | 1) | 2) | Copies | (ng) | (ng) | RNA |
| Subcutaneous | 35.66, 34.05 | 3.76 | 10.2 | 6.98 | 3.06 | 16.34 | 114.05 |
| Adipocytes Zenbio | | | | | | | |
| Subcutaneous Adipose | 40, 36 | 0.17 | 3.07 | 1.62 | 0.96 | 52.36 | 84.82 |
| Zenbio | | | | | | | |
| Adrenal Gland Clontech | 40, 40 | 0 | 0 | 0.00 | 0.61 | 81.97 | 0.00 |
| Whole Brain Clontech | 26.32, 26.41 | 1192.66 | 1124.69 | 1158.68 | 7.24 | 6.91 | 8001.90 |
| Fetal Brain Clontech | 40, 35.84 | 0 | 3.38 | 1.69 | 0.48 | 103.95 | 175.68 |
| Cerebellum Clontech | 34.51, 34.28 | 7.68 | 8.8 | 8.24 | 2.17 | 23.04 | 189.86 |
| Cervix | 40, 34.34 | 3.17 | 8.5 | 5.84 | 2.42 | 20.66 | 120.56 |
| Colon | 33.67, 35.6 | 12.86 | 3.91 | 8.39 | 2.71 | 18.45 | 154.70 |
| Endometrium | 35.32, 34.43 | 4.66 | 8.05 | 6.36 | 0.73 | 68.21 | 433.49 |
| Esophagus | 34.27, 35.14 | 8.86 | 5.19 | 7.03 | 1.37 | 36.50 | 256.39 |
| Heart Clontech | 40, 35.05 | 0 | 5.5 | 2.75 | 1.32 | 37.88 | 104.17 |
| Hypothalamus | 40, 40 | 0 | 0 | 0.00 | 0.32 | 155.28 | 0.00 |
| Ileum | 35.29, 33.68 | 4.74 | 12.8 | 8.77 | 2.58 | 19.38 | 169.96 |
| Jejunum | 31.23, 30.98 | 57.65 | 67.22 | 62.44 | 6.60 | 7.58 | 472.99 |
| Kidney | 34.67, 34.21 | 6.95 | 9.2 | 8.08 | 2.12 | 23.58 | 190.45 |
| Liver | 30.76, 30.65 | 77.12 | 82.56 | 79.84 | 1.50 | 33.33 | 2661.33 |
| Fetal Liver Clontech | 26.8, 27.1 | 885.14 | 734.31 | 809.73 | 10.40 | 4.81 | 3892.91 |
| Lung | 40, 40 | 0 | 0.17 | 0.09 | 2.57 | 19.46 | 1.65 |
| Mammary Gland | 31.28, 31.37 | 56.1 | 52.95 | 54.53 | 13.00 | 3.85 | 209.71 |
| Clontech | | | | | | | |
| Myometrium | 34.16, 36.28 | 9.48 | 2.57 | 6.03 | 2.34 | 21.37 | 128.74 |
| Omentum | 34.18, 33.42 | 9.38 | 15 | 12.19 | 3.94 | 12.69 | 154.70 |
| Ovary | 34.21, 34.18 | 9.24 | 9.39 | 9.32 | 4.34 | 11.52 | 107.32 |
| Pancreas | 40, 40 | 0 | 0.14 | 0.07 | 0.81 | 61.80 | 4.33 |
| Head of Pancreas | 40, 35.02 | 0 | 5.59 | 2.80 | 1.57 | 31.85 | 89.01 |
| Parotid Gland | 31.23, 31.9 | 57.68 | 38.33 | 48.01 | 5.48 | 9.12 | 438.00 |
| Placenta Clontech | 31.77, 33.13 | 41.33 | 17.94 | 29.64 | 5.26 | 9.51 | 281.70 |
| Prostate | 39.72, 35.03 | 0.31 | 5.56 | 2.94 | 3.00 | 16.67 | 48.92 |
| Rectum | 35.36, 34.34 | 4.53 | 8.5 | 6.52 | 1.23 | 40.65 | 264.84 |
| Salivary Gland | 30.52, 30.54 | 89.5 | 88.43 | 88.97 | 7.31 | 6.84 | 608.52 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 39.27 | 0 | 0.41 | 0.21 | 0.98 | 51.07 | 10.47 |
| Clontech | | | | | | | |
| Spleen | 34.21, 33.54 | 9.2 | 13.91 | 11.56 | 4.92 | 10.16 | 117.43 |
| Stomach | 35.05, 33.62 | 5.51 | 13.22 | 9.37 | 2.73 | 18.32 | 171.52 |
| Testis Clontech | 40, 40 | 0 | 0 | 0.00 | 0.57 | 87.87 | 0.00 |
| Thymus Clontech | 28.56, 28.44 | 299.45 | 322.02 | 310.74 | 9.89 | 5.06 | 1570.96 |
| Thyroid | 31.65, 32.3 | 44.76 | 29.81 | 37.29 | 2.77 | 18.05 | 673.01 |
| Trachea Clontech | 32.3, 31.9 | 29.9 | 38.28 | 34.09 | 9.71 | 5.15 | 175.54 |
| Urinary Bladder | 34.34, 35.02 | 8.49 | 5.59 | 7.04 | 5.47 | 9.14 | 64.35 |
| Uterus | 33.07, 34.56 | 18.62 | 7.45 | 13.04 | 5.34 | 9.36 | 122.05 |
| genomic | 25.84 | 1597.08 | | | | | |
| b-actin | 27.32 | 643.56 | | | | | |
| 1.00E+05 | 19.22 | 100000 | | | | | |
| 1.00E+05 | 19.33 | 100000 | | | | | |
| 1.00E+04 | 22.48 | 10000 | | | | | |
| 1.00E+04 | 22.95 | 10000 | | | | | |
| 1.00E+03 | 26.19 | 1000 | | | | | |
| 1.00E+03 | 26.37 | 1000 | | | | | |
| 1.00E+02 | 31.23 | 100 | | | | | |
| 1.00E+02 | 30.48 | 100 | | | | | |
| 1.00E+01 | 32.76 | 10 | | | | | |
| 1.00E+01 | 35.02 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | Reg | | | mRNA | | Fold |
| | number | | Mean | detected/ | | Change in |
| Sample | (GSK | | GOI | 50 ng total | | Disease |
| sbg102200MCTc | identifier) | Ct | copies | RNA | Sample | Population |
| colon normal GW98-167 | 21941 | 26.48 | 1723.59 | 3447.18 | colon normal | |
| colon tumor GW98-166 | 21940 | 26.06 | 2195.04 | 4390.08 | colon tumor | 1.27 |
| colon normal GW98-178 | 22080 | 29.03 | 389.88 | 779.76 | colon normal | |
| colon tumor GW98-177 | 22060 | 27.39 | 1015.65 | 2031.30 | colon tumor | 2.61 |
| colon normal GW98-561 | 23514 | 26.74 | 1478.76 | 2957.52 | colon normal | |
| colon tumor GW98-560 | 23513 | 26.37 | 1831.8 | 3663.60 | colon tumor | 1.24 |
| colon normal GW98-894 | 24691 | 25.58 | 2918.02 | 5836.04 | colon normal | |
| colon tumor GW98-893 | 24690 | 25 | 4089.75 | 8179.50 | colon tumor | 1.40 |
| lung normal GW98-3 | 20742 | 24.59 | 5183.31 | 10366.62 | lung normal | |
| lung tumor GW98-2 | 20741 | 24.94 | 4232.23 | 8464.46 | lung tumor | −1.22 |
| lung normal GW97-179 | 20677 | 25.73 | 2672.73 | 5345.46 | lung normal | |
| lung tumor GW97-178 | 20676 | 25.36 | 3307.37 | 6614.74 | lung tumor | 1.24 |
| lung normal GW98-165 | 21922 | 26.13 | 2109.28 | 4218.56 | lung normal | |
| lung tumor GW98-164 | 21921 | 25.54 | 2973.82 | 5947.64 | lung tumor | 1.41 |
| lung normal GW98-282 | 22584 | 27.08 | 1212.64 | 2425.28 | lung normal | |
| lung tumor GW98-281 | 22583 | 27.45 | 979.82 | 1959.64 | lung tumor | −1.24 |
| breast normal GW00-392 | 28750 | 26.68 | 1536.57 | 1536.57 | breast normal | |
| breast tumor GW00-391 | 28746 | 26.58 | 1626.58 | 3253.16 | breast tumor | 2.12 |
| breast normal GW00-413 | 28798 | 31.71 | 81.9 | 81.90 | breast normal | |
| breast tumor GW00-412 | 28797 | 26.57 | 1632 | 3264.00 | breast tumor | 39.85 |
| breast normal GW00- | 27592-95 | 32.52 | 51.1 | 51.10 | breast normal | |
| 235:238 | | | | | | |
| breast tumor GW00- | 27588-91 | 29.67 | 268.7 | 268.70 | breast tumor | 5.26 |
| 231:234 | | | | | | |
| breast normal GW98-621 | 23656 | 26.48 | 1727.44 | 3454.88 | breast normal | |
| breast tumor GW98-620 | 23655 | 25.65 | 2793.6 | 5587.20 | breast tumor | 1.62 |
| brain normal BB99-542 | 25507 | 28.62 | 494 | 988.00 | brain normal | |
| brain normal BB99-406 | 25509 | 29.45 | 304.68 | 609.36 | brain normal | |
| brain normal BB99-904 | 25546 | 30.08 | 211.25 | 422.50 | brain normal | |
| brain stage 5 ALZ BB99- | 25502 | 28.75 | 458.64 | 917.28 | brain stage 5 | 1.36 |
| 874 | | | | | ALZ | |
| brain stage 5 ALZ BB99- | 25503 | 26.86 | 1383.71 | 2767.42 | brain stage 5 | 4.11 |
| 887 | | | | | ALZ | |
| brain stage 5 ALZ BB99- | 25504 | 28.02 | 702.59 | 1405.18 | brain stage 5 | 2.09 |
| 862 | | | | | ALZ | |
| brain stage 5 ALZ BB99- | 25542 | 29.57 | 284.31 | 568.62 | brain stage 5 | −1.18 |
| 927 | | | | | ALZ | |
| CT lung KC | normal | 26.58 | 1624.29 | 3248.58 | CT lung | |
| lung 26 KC | normal | 34.19 | 19.27 | 19.27 | lung 26 | |
| lung 27 KC | normal | 32.45 | 53.23 | 53.23 | lung 27 | |
| lung 24 KC | COPD | 33 | 38.6 | 38.60 | lung 24 | −21.75 |
| lung 28 KC | COPD | 32.24 | 59.95 | 59.95 | lung 28 | −14.01 |
| lung 23 KC | COPD | 32.87 | 41.63 | 41.63 | lung 23 | −20.17 |
| lung 25 KC | normal | 33.04 | 37.52 | 37.52 | lung 25 | |
| asthmatic lung ODO3112 | 29321 | 30.13 | 205.46 | 205.46 | asthmatic | −4.09 |
| | | | | | lung | |
| asthmatic lung ODO3433 | 29323 | 27.82 | 788.82 | 1577.64 | asthmatic | 1.88 |
| | | | | | lung | |
| asthmatic lung ODO3397 | 29322 | 25.17 | 3695.43 | 7390.86 | asthmatic | 8.80 |
| | | | | | lung | |
| asthmatic lung ODO4928 | 29325 | 27.6 | 894.3 | 1788.60 | asthmatic | 2.13 |
| | | | | | lung | |
| endo cells KC | control | 28.2 | 633.43 | 633.43 | endo cells | |
| endo VEGF KC | | 28.86 | 429.51 | 429.51 | endo VEGF | −1.47 |
| endo bFGF KC | | 28.97 | 403.08 | 403.08 | endo bFGF | −1.57 |
| heart Clontech | normal | 28.83 | 437.62 | 875.24 | heart | |
| heart (T-1) ischemic | 29417 | 28.42 | 557.54 | 1115.08 | heart T-1 | 1.27 |
| heart (T-14) non- | 29422 | 27.72 | 835.11 | 1670.22 | heart T-14 | 1.91 |
| obstructive DCM | | | | | | |
| heart (T-3399) DCM | 29426 | 28.63 | 493.01 | 986.02 | heart T-3399 | 1.13 |
| adenoid GW99-269 | 26162 | 27 | 1269.75 | 2539.50 | adenoid | |
| tonsil GW98-280 | 22582 | 26.33 | 1876.29 | 3752.58 | tonsil | |
| T cells PC00314 | 28453 | 29.15 | 363.35 | 726.70 | T cells | |
| PBMNC | | 33.05 | 37.41 | 37.41 | PBMNC | |
| monocyte | | 31.49 | 92.84 | 185.68 | monocyte | |
| B cells PC00665 | 28455 | 26.5 | 1700.87 | 3401.74 | B cells | |
| dendritic cells 28441 | | 24.2 | 6511.17 | 13022.34 | dendritic cells | |
| neutrophils | 28440 | 27.01 | 1262.74 | 1262.74 | neutrophils | |
| eosinophils | 28446 | 29.23 | 347.08 | 694.16 | eosinophils | |
| BM unstim | | 30.85 | 135.01 | 135.01 | BM unstim | |
| BM stim | | 28.68 | 478.5 | 478.50 | BM stim | 3.54 |
| osteo dif | | 31.03 | 121.2 | 121.20 | osteo dif | 3.93 |
| osteo undif | | 33.38 | 30.85 | 30.85 | osteo undif | |
| chondrocytes | | 26.63 | 1579.73 | 3949.33 | chondrocytes | |
| OA Synovium IP12/01 | 29462 | 29.11 | 371.98 | 371.98 | OA | |
| | | | | | Synovium | |
| OA Synovium NP10/01 | 29461 | 29.45 | 304.55 | 609.10 | OA | |
| | | | | | Synovium | |
| OA Synovium NP57/00 | 28464 | 27.83 | 784.87 | 1569.74 | OA | |
| | | | | | Synovium | |
| RA Synovium NP03/01 | 28466 | 27.31 | 1063.77 | 2127.54 | RA Synovium | |
| RA Synovium NP71/00 | 28467 | 27.08 | 1217.21 | 2434.42 | RA Synovium | |
| RA Synovium NP45/00 | 28475 | 26.6 | 1606.41 | 3212.82 | RA Synovium | |
| OA bone (biobank) | 29217 | 28.65 | 485.63 | 485.63 | OA bone | |
| | | | | | (biobank) | |
| OA bone Sample 1 | J. Emory | 28.78 | 451.74 | 903.48 | OA bone | |
| OA bone Sample 2 | J. Emory | 28.27 | 607.15 | 1214.30 | OA bone | |
| Cartilage (pool) | Normal | 29.42 | 310.76 | 621.52 | Cartilage | |
| | | | | | (pool) | |
| Cartilage (pool) | OA | 30.09 | 209.7 | 419.40 | Cartilage | −1.48 |
| | | | | | (pool) | |
| PBL unifected | 28441 | 23.85 | 7997.03 | 15994.06 | PBL | |
| | | | | | unifected | |
| PBL HIV IIIB | 28442 | 24.85 | 4447.34 | 8894.68 | PBL HIV | −1.80 |
| | | | | | IIIB | |
| MRC5 uninfected (100%) | 29158 | 27.02 | 1258.46 | 2516.92 | MRC5 | |
| | | | | | uninfected | |
| | | | | | (100%) | |
| MRC5 HSV strain F | 29178 | 29.6 | 278.84 | 557.68 | MRC5 HSV | −4.51 |
| | | | | | strain F | |
| W12 cells | 29179 | 27.21 | 1122.77 | 2245.54 | W12 cells | |
| Keratinocytes | 29180 | 25.64 | 2815.12 | 5630.24 | Keratinocytes | |
| B-actin control | | 27.78 | 807.72 | | | |
| genomic | | 27.04 | 1246.22 | | | |
| 1.00E+05 | | 19.69 | 100000 | | | |
| 1.00E+05 | | 20.01 | 100000 | | | |
| 1.00E+04 | | 23.15 | 10000 | | | |
| 1.00E+04 | | 23.2 | 10000 | | | |
| 1.00E+03 | | 27.02 | 1000 | | | |
| 1.00E+03 | | 26.76 | 1000 | | | |
| 1.00E+02 | | 31.45 | 100 | | | |
| 1.00E+02 | | 32.39 | 100 | | | |
| 1.00E+01 | | 35.72 | 10 | | | |
| 1.00E+01 | | 34.74 | 10 | | | |
| 1.00E−00 | | 40 | 0 | | | |
| 1.00E−00 | | 40 | 0 | | | |
| NTC | | 40 | 0 | | | |

Gene Name sbg102200MCTc

| | Fold Change in Disease |
|---|---|
| | Population Relative to |
| Disease tissues | Normal |
| colon tumor | 1.27 |
| colon tumor | 2.61 |
| colon tumor | 1.24 |
| colon tumor | 1.40 |
| lung tumor | −1.22 |
| lung tumor | 1.24 |
| lung tumor | 1.41 |
| lung tumor | −1.24 |
| breast tumor | 2.12 |
| breast tumor | 39.85 |
| breast tumor | 5.26 |
| breast tumor | 1.62 |
| brain stage 5 ALZ | 1.36 |
| brain stage 5 ALZ | 4.11 |
| brain stage 5 ALZ | 2.09 |
| brain stage 5 ALZ | −1.18 |
| lung 24 | −21.75 |
| lung 28 | −14.01 |
| lung 23 | −20.17 |
| asthmatic lung | −4.09 |
| asthmatic lung | 1.88 |
| asthmatic lung | 8.80 |
| asthmatic lung | 2.13 |
| endo VEGF | −1.47 |
| endo bFGF | −1.57 |
| heart T-1 | 1.27 |
| heart T-14 | 1.91 |
| heart T-3399 | 1.13 |
| BM stim | 3.54 |
| osteo dif | 3.93 |
| Cartilage (pool) | −1.48 |
| PBL HIV IIIB | −1.80 |
| MRC5 HSV strain F | −4.51 |

Gene Name sbg18525LRRb, SBhACRP30c, and sbg123493SLITc

Quantitative, tissue-specific, mRNA expression patterns of these three genes were measured using SYBR-Green Quantitative PCR (Applied Biosystems, Foster City, Calif.; see Schmittgen T. D. et al., Analytical Biochemistry 285: 194-204, 2000) or TaqMan PCR (Perkin Elmer, see Lie et al. Current Opinion in Biotechnology 9:43-48, 1998; Gibson et al., Genome Methods 6:995-1001, 1996) and human cDNAs prepared from various human tissues. Gene-specific PCR primers were designed using the first nucleic acid sequence listed in the Sequence List for each gene. Results are presented as the number of copies of each specific gene's mRNA detected in 1 ng mRNA pool from each tissue. Two replicate mRNA measurements were made from each tissue RNA.

| | Tissue-Specific mRNA Expression | | | | | | | | | |
|---|---|---|---|---|---|---|---|---|---|---|
| | (copies per ng mRNA; avg. ± range for 2 data points per tissue) | | | | | | | | | |
| | | | | | | Skeletal | | Spleen/ | | |
| Gene Name | Brain | Heart | Lung | Liver | Kidney | muscle | Intestine | lymph | Placenta | Testis |
| sbg18525LRRb | 4290 ± | 367 ± | 47 ± | 7 ± | 263 ± | 69 ± | 401 ± | 39 ± | 119 ± | 307 ± |
| | 157 | 6 | 4 | 0 | 10 | 7 | 62 | 3 | 17 | 1 |
| SBhACRP30c | 10751 ± | 7443 ± | 9900 ± | 6463 ± | 8530 ± | 7638 ± | 6040 ± | 8912 ± | 8931 ± | 8098 ± |
| | 954 | 294 | 780 | 45 | 225 | 405 | 438 | 1021 | 617 | 612 |
| sbg123493SLITc | 9 ± | 70 ± | 13 ± | 1 ± | 41 ± | 132 ± | 6 ± | 5 ± | 9 ± | 959 ± |
| | 3 | 31 | 3 | 1 | 16 | 21 | 2 | 10 | 4 | 80 |

TABLE V
Additional diseases based on mRNA expression in specific tissues

| Tissue Exp. | Additional Diseases |
|---|---|
| Brain | Neurological and psychiatric diseases, |
| | including Alzheimers, parasupranuclear |
| | palsey, Huntington's disease, myotonic |
| | dystrophy, anorexia, depression, |
| | schizophrenia, headache, amnesias, anxiety |
| | disorders, sleep disorders, multiple |
| | sclerosis |
| Heart | Cardiovascular diseases, including |
| | congestive heart failure, dilated |
| | cardiomyopathy, cardiac arrhythmias, |
| | Hodgson's Disease, myocardial |
| | infarction, cardiac arrhythmias |
| Lung | Respiratory diseases, including asthma, |
| | Chronic Obstructive Pulmonary |
| | Disease, cystic fibrosis, acute bronchitis, |
| | adult respiratory distress syndrome |
| Liver | Dyslipidemia, hypercholesterolemia, |
| | hypertriglyceridemia, cirrhosis, hepatic |
| | encephalopathy, fatty hepatocirrhosis, |
| | viral and nonviral hepatitis, Type II |
| | Diabetes Mellitis, impaired glucose tolerance |
| Kidney | Renal diseases, including acute and chronic |
| | renal failure, acute tubular necrosis, |
| | cystinuria, Fanconi's Syndrome, |
| | glomerulonephritis, renal cell carcinoma, |
| | renovascular hypertension |
| Skeletal | Eulenburg's Disease, hypoglycemia, obesity, |
| muscle | tendinitis, periodic paralyses, |
| | malignant hyperthermia, paramyotonia congenita, |
| | myotonia congenita |
| Intestine | Gastrointestinal diseases, including Myotonia |
| | congenita, Ileus, Intestinal |
| | Obstruction, Tropical Sprue, Pseudomembranous |
| | Enterocolitis |
| Spleen/ | Lymphangiectasia, hypersplenism, angiomas, |
| lymph | ankylosing spondylitis, Hodgkin's |
| | Disease, macroglobulinemia, malignant lymphomas, |
| | rheumatoid arthritis |
| Placenta | Choriocarcinoma, hydatidiform mole, |
| | placenta previa |
| Testis | Testicular cancer, male reproductive diseases, |
| | including low testosterone and male infertility |
| Pancreas | Diabetic ketoacidosis, Type 1 & 2 diabetes, |
| | obesity, impaired glucose tolerance |

What is claimed is:

1. An isolated polypeptide selected from the group consisting of:
(a) an isolated polypeptide encoded by a polynucleotide comprising a sequence set forth in Table I;
(b) an isolated polypeptide comprising a polypeptide sequence set forth in Table I; and
(c) a polypeptide sequence of a gene set forth in Table I.

2. An isolated polynucleotide selected from the group consisting of:
(a) an isolated polynucleotide comprising a polynucleotide sequence set forth in Table I;
(b) an isolated polynucleotide of a gene set forth in Table I;
(c) an isolated polynucleotide comprising a polynucleotide sequence encoding a polypeptide set forth in Table I;
(d) an isolated polynucleotide encoding a polypeptide set forth in Table I;
(e) a polynucleotide which is an RNA equivalent of the polynucleotide of (a) to (d);
or a polynucleotide sequence complementary to said isolated polynucleotide.

3. An expression vector comprising a polynucleotide capable of producing a polypeptide of claim 1 when said expression vector is present in a compatible host cell.

4. A process for producing a recombinant host cell which comprises the step of introducing an expression vector comprising a polynucleotide capable of producing a polypeptide of claim 1 into a cell such that the host cell, under appropriate culture conditions, produces said polypeptide.

5. A recombinant host cell produced by the process of claim 4.

6. A membrane of a recombinant host cell of claim 5 expressing said polypeptide.

7. A process for producing a polypeptide which comprises culturing a host cell of claim 5 under conditions sufficient for the production of said polypeptide and recovering said polypeptide from the culture.
